SEQUENCE LISTING (4 sequences)

SEQ ID NO: 1 - DNA, 40 bp, synthetic construct
  source 1..40: mol_type = other DNA
  modified_base 40: mod_base = OTHER; note = Uridine
  modified_base 39: mod_base = OTHER; note = Uridine
cgctaacaatacaagaatcatacaacagaatagtccagtt

SEQ ID NO: 2 - DNA, 40 bp, synthetic construct
  source 1..40: mol_type = other DNA
aactggactattctgttgtatgattcttgtattgttagcg

SEQ ID NO: 3 - DNA, 40 bp, synthetic construct; identical to SEQ ID NO: 1
  source 1..40: mol_type = other DNA
  modified_base 39: mod_base = OTHER; note = Uridine
  modified_base 40: mod_base = um

SEQ ID NO: 4 - DNA, 40 bp, synthetic construct; identical to SEQ ID NO: 2
  source 1..40: mol_type = other DNA
  modified_base 40: mod_base = gm

(19) United States
(12) Patent Application Publication
(10) Pub. No.: US 2025/0172566 A1
(43) Pub. Date: May 29, 2025

(54) SACCHARIDE SENSING SYSTEMS

(71) Applicant: CARBOMETRICS LIMITED, Bristol (GB)

(72) Inventors: Andrew Michael CHAPMAN, Bristol (GB); Anthony DAVIS, Bristol (GB); Daniel Jamie LEONARD, Bristol (GB); Michael Glen ORCHARD, Baydon, Marlborough (GB); MIchael Roger TOMSETT, Bristol (GB); Robert TROMANS, Bristol (GB)

(21) Appl. No.: 18/833,838

(22) PCT Filed: Jan. 26, 2023
(86) PCT No.: PCT/GB2023/050178

§ 371 Date: Jul. 26, 2024

(30) Foreign Application Priority Data
Jan. 26, 2022 (GB) 2201000.3

(51) Int. Cl. G01N 33/66; A61B 5/145; A61B 5/1459; A61B 5/1473; C07D 487/08; C07D 519/00; C07H 15/26; G01N 21/64

(52) U.S. Cl. CPC G01N 33/66; A61B 5/14532; A61B 5/1459; A61B 5/14735; C07D 487/08; C07D 519/00; C07H 15/26; G01N 21/6428; G01N 2021/6439

(57) ABSTRACT

The present invention relates to saccharide sensing systems including macrocyclic compounds which are capable of binding to a target saccharide (e.g. glucose) and a detectable reporter for providing a detectable signal based on the binding of the target saccharide by the macrocyclic compound.

The present invention relates to saccharide sensing systems including macrocyclic compounds which are capable of binding to a target saccharide (e.g. glucose) and a detectable reporter for providing a detectable signal based on the binding of the target saccharide by the macrocyclic compound.

### BACKGROUND

The detection of saccharides, in particular glucose, and their concentration in solution have significant medical and non-medical applications. For example, the reliable detection of glucose in an individual's bloodstream has significant importance in the diagnosis, monitoring and treatment of diabetes.

Recently macrocyclic compounds have been developed to selectively bind to a target saccharide in WO 2018/167503, and such macrocyclic compounds show good saccharide binding.

There is a desire to provide saccharide sensing systems that can indicate the presence of saccharides.

### SUMMARY

At its most general, the present invention provides a saccharide sensing system including a macrocyclic compound capable of binding to a target saccharide (e.g. glucose) and a detectable reporter for providing a detectable signal. The macrocycle typically has a saccharide-binding cavity for binding the saccharide. The detectable signal may change depending on the environment of the detectable reporter, and in particular whether or not the detectable reporter is associated with the saccharide-binding compound. The detectable reporter is typically associated with a cavity-binding moiety that may compete with the target saccharide for binding of the saccharide-binding cavity of the macrocyclic compound.

In some aspects of the present invention, the detectable reporter includes a fluorescent reporter moiety to provide a detectable signal based on the binding of the target saccharide. In other aspects, the system includes a redox moiety as the detectable reporter and an electrode.

The present inventors have designed a number of saccharide (e.g. glucose) sensing systems based on this premise.

In a first aspect, the present invention provides a saccharide sensing system, the sensing system includes a saccharide-binding compound as defined herein having a saccharide-binding cavity for binding a target saccharide and a detectable reporter for providing a detectable signal, and wherein the sensing system includes an aqueous solution or dispersion including the saccharide-binding compound and the detectable reporter, the detectable reporter is a displaceable cavity-binding ligand including a fluorescent reporter moiety and a cavity-binding moiety for binding the cavity of the saccharide-binding compound.

In a second aspect, the present invention provides a saccharide sensing system, the sensing system includes a saccharide-binding compound as defined herein having a saccharide-binding cavity for binding a target saccharide and a detectable reporter for providing a detectable signal based on the detection or change of the target saccharide in an aqueous environment, and wherein the sensing system includes an electrode in fluid contact with an aqueous solution and the aqueous solution containing the saccharide-binding compound and the detectable reporter, the detectable reporter is a displaceable cavity-binding ligand including a redox moiety capable of reversible oxidation and a cavity-binding moiety for binding the cavity of the saccharide-binding compound.

In a third aspect, the present invention provides a saccharide sensing system, the sensing system includes a saccharide-binding compound as defined herein having a saccharide-binding cavity for binding a target saccharide and a detectable reporter for providing a detectable signal based on the detection or change of the target saccharide in an aqueous environment, and wherein the sensing system includes a semi-solid or solid support, wherein the saccharide-binding compound is immobilized on the semi-solid or solid support, the detectable reporter is a displaceable cavity-binding ligand independently immobilized on the semi-solid or solid support, where the displaceable cavity-binding ligand includes a fluorescent reporter moiety and a cavity-binding moiety for binding the cavity of the saccharide-binding compound.

In a fourth aspect, the present invention provides a saccharide sensing system, the sensing system includes a saccharide-binding compound as defined herein having a saccharide-binding cavity for binding a target saccharide and a detectable reporter for providing a detectable signal based on the detection or change of the target saccharide in an aqueous environment, and wherein the saccharide-binding compound includes a cavity-binding tether group attached to a macrocyle of the saccharide-binding compound, the cavity-binding tether group including a fluorescent reporter moiety and a displaceable cavity-binding moiety for binding the cavity of a saccharide-binding compound such that the saccharide binding compound includes the detectable reporter of the system.

Without wishing to be bound by theory, the systems described herein include a saccharide-binding compound to bind a target saccharide (e.g. glucose) and a cavity-binding portion associated with a fluorescent reporter moiety, wherein the cavity-binding portion may compete with the target saccharide for cavity-binding and may allow detection of the presence or concentration of the target saccharide based on changes of the detectable reporter associated with the cavity-binding moiety.

In a fifth aspect, the present invention provides a device including any one of the saccharide sensing systems of the first to fourth aspects.

In a sixth aspect, the present invention provides a method of detecting a target saccharide in a target aqueous environment using one of the saccharide sensing systems of the first to fourth aspects includes:
(i) contacting the saccharide sensing system with the target aqueous environment; and
(ii) measuring a detectable signal from the detectable reporter of the system.

In a seventh aspect, the present invention provides use of any one of the saccharide sensing systems of the first to fourth aspect to detect a target saccharide in a target aqueous environment.

In an eight aspect, the present invention provides a saccharide-binding compound as defined herein adapted to be immobilized on a solid or semi-solid support.

In a ninth aspect, the present invention provides a displaceable cavity-binding ligand including a fluorescent reporter moiety and a cavity-binding moiety for binding the cavity of a saccharide-binding compound as defined herein, wherein the displaceable cavity-binding ligand is adapted to be immobilized on a solid or semi-solid support.

In a tenth aspect, the present invention provides a method of immobilizing a saccharide-binding compound according to the eighth aspect and/or a displaceable cavity-binding ligand according to the ninth aspect to a solid or semi-solid support.

In particular embodiments of all of the aspects of the present invention, the saccharide (or target saccharide) is glucose.

### DETAILED DESCRIPTION

The present invention will now be described in detail with reference to the accompanying Figures.

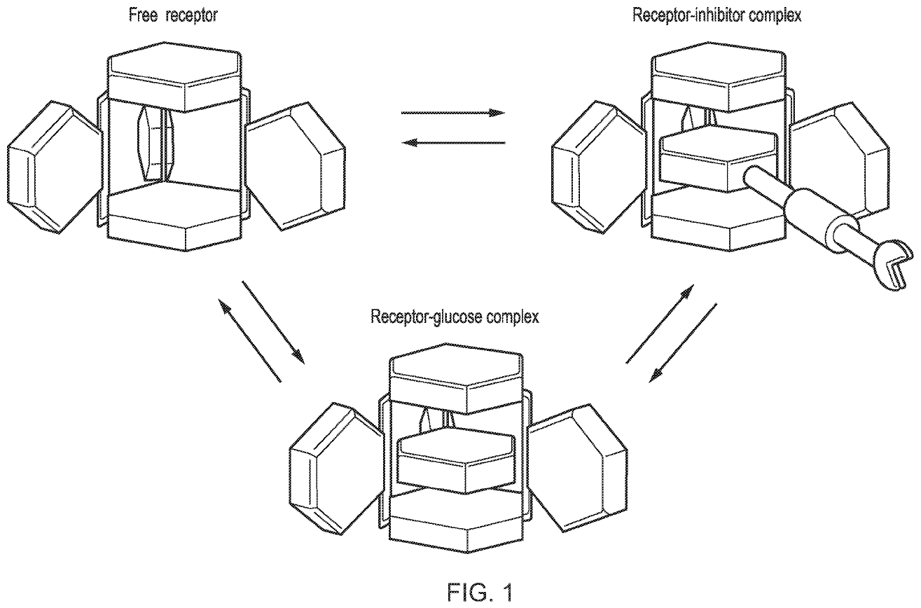

FIG. 1 shows schematic of an equilibrium between an unbound saccharide-binding compound described herein (top left), the saccharide-binding compound bound with a displaceable cavity binding ligand as described herein (top right) and the saccharide-binding compound bound with the target saccharide (bottom).

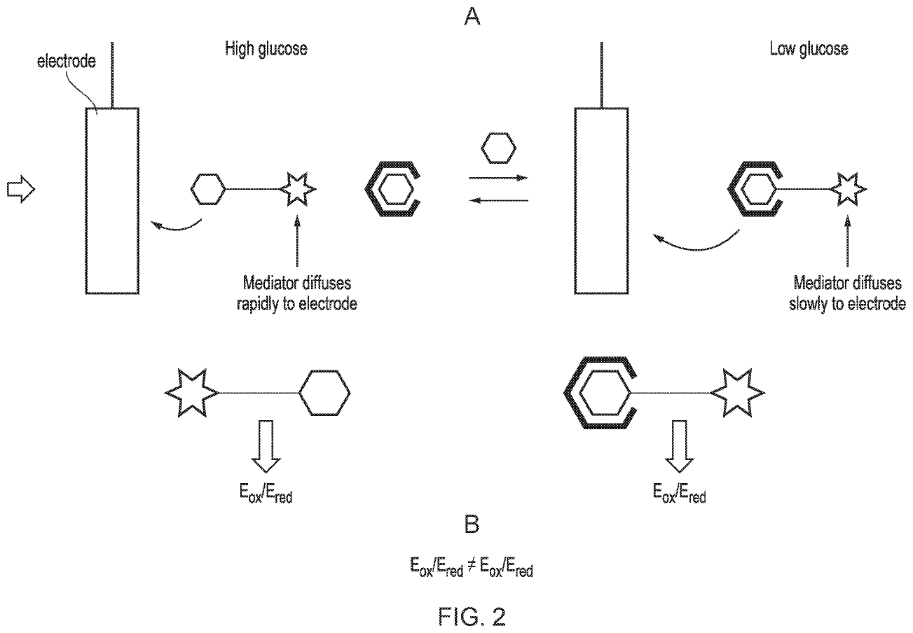

FIG. 2 shows a schematic of the system of the second aspect with (A) fast diffusion of the displaceable cavity-binding ligand to an electrode when not bound to the saccharide-binding compound and slow diffusion of the displaceable cavity-binding ligand to an electrode when bound to the saccharide-binding compound; and (B) a difference in the reduction and/or oxidation potential of the redox moiety when displaceable cavity-binding ligand is bound or not bound to the saccharide-binding compound.

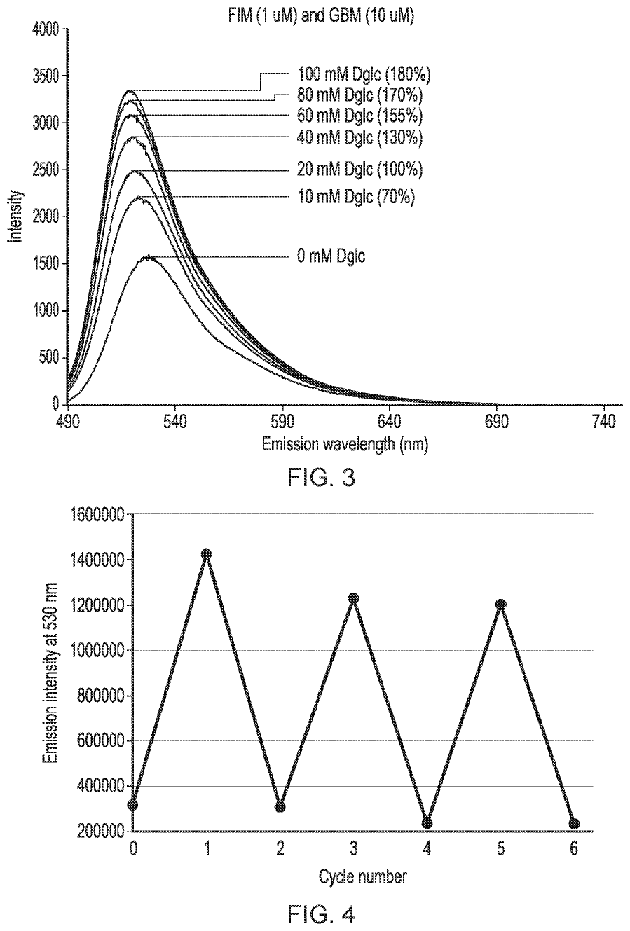

FIG. 3 shows an example glucose responsive fluorescence assay.

FIG. 4 shows an example of cycling of a hydrogel containing GBM2 and FIM4 pre-incorporated into the hydrogel.

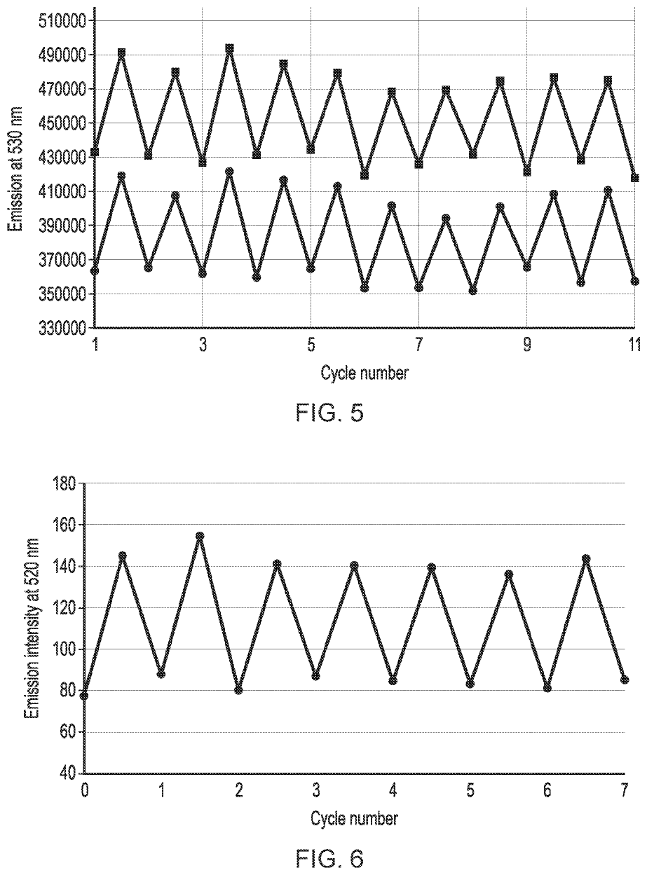

FIG. 5 shows an example of cycling of hydrogel D containing GBM1 and FIM1 pre-incorporated into the hydrogel.

FIG. 6 shows an example of cycling of hydrogel E containing GBM36 pre-incorporated into the hydrogel.

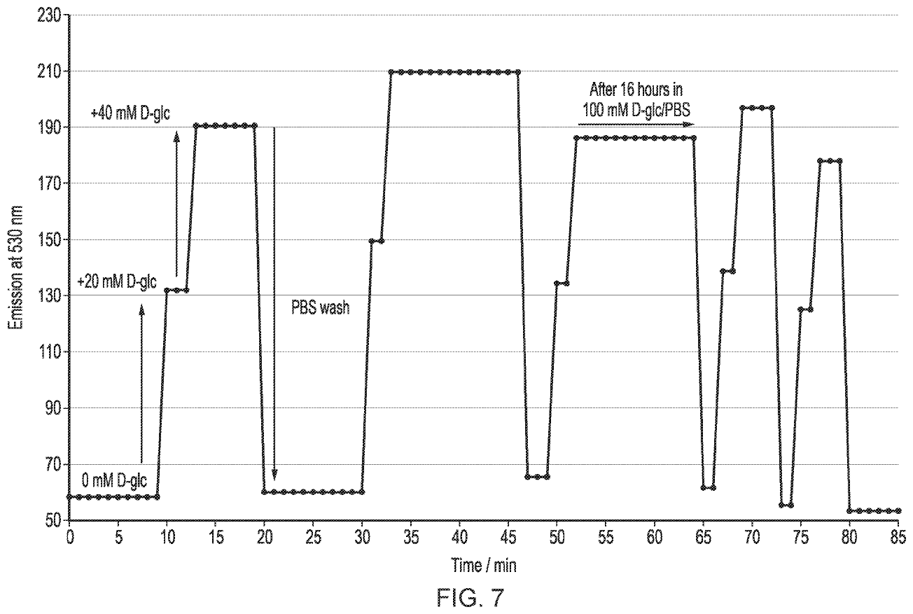

FIG. 7 shows an example cycling of hydrogel F containing FIM41 (5 μM) and GBM15 (50 μM) incorporated into the hydrogel.

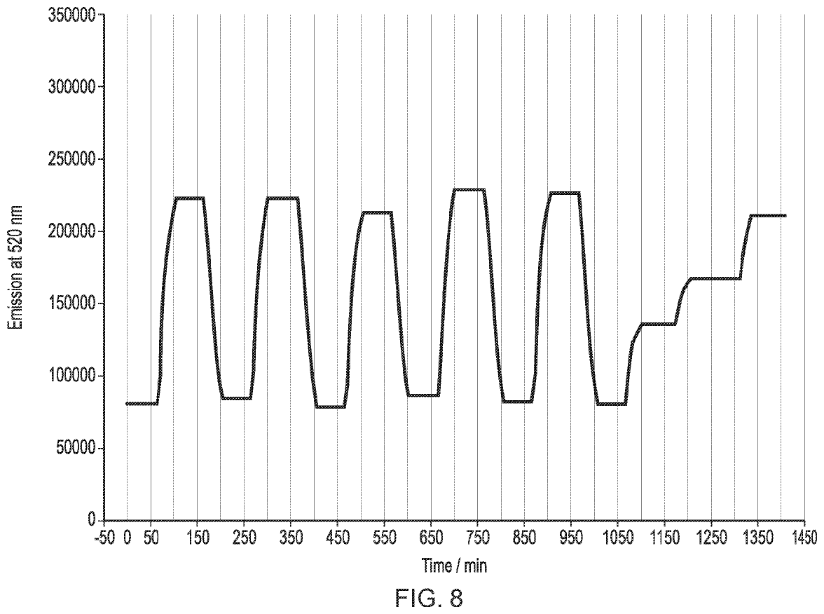

FIG. 8 shows an example cycling of FIM61 (1 μM) and GBM30 (100 uM) while encapsulated within a cellulose acetate membrane (MWCO 10 kDa).

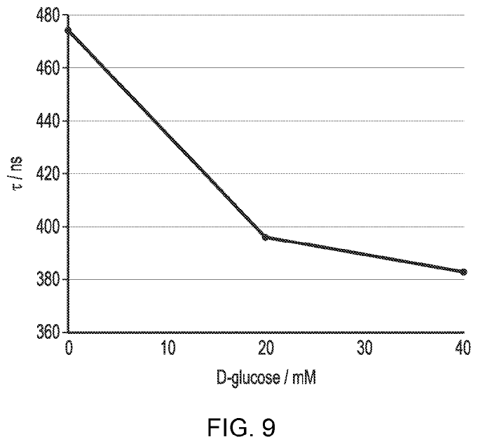

FIG. 9 is a graphical representation showing changes in fluorescence lifetime for assay containing FIM15 and GBM15 at various concentrations of D-glucose.

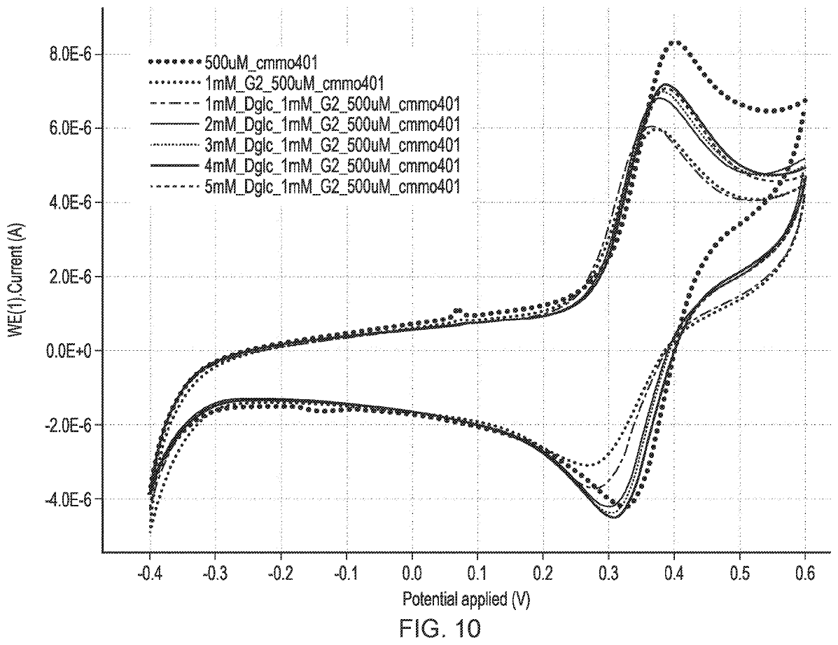

FIG. 10 shows an example glucose responsive cyclic voltammetry for assay comprised of GBM15 and FIM38 in PBS.

Key components in each of the aspects of the present invention are the saccharide-binding compound and the detectable reporter.

### Saccharide-Binding Compound

The saccharide-binding compound may be a compound of Formula (I) or a salt, hydrate and/or solvate thereof, as shown below:

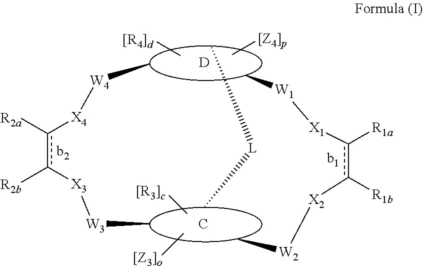

Wherein:
bonds b₁ and b₂ are independently selected from a single bond or double bond; R₁ₐ, R_{1b}, R₂ₐ and R_{2b} are independently selected from hydrogen, carbonyl, (1-8C)alkyl, (3-10C)cycloalkyl, aryl, heteroaryl and heterocyclyl, each of which, other than hydrogen and carbonyl, is optionally substituted by one or more substituent groups selected from (1-4C)alkyl, halo, (1-4C)haloalkyl, (1-4C)haloalkoxy, (1-4C)alkoxy, (1-4C)alkylamino, amino, cyano, hydroxyl, carboxy, carbamoyl, sulfamoyl, mercapto and a hydrophilic substituent group; or R₁ₐ and R_{1b} are linked so as to form a group of the formula:

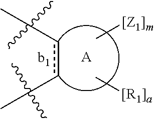

and/or R₂ₐ and R_{2b} are linked so as to form a group of the formula:

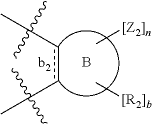

wherein:
[symbol] denotes the point of attachment;
bonds b₁ and b₂ are as described above;
Rings A and B are independently selected from aryl, heteroaryl heterocyclyl, cycloalkyl and cycloalkenyl;
R₁ and R₂ are independently selected from (1-6C)alkyl, halo, (1-4C)haloalkyl, (1-4C)haloalkoxy, (1-6C)alkoxy, (1-4C)alkylamino, amino, cyano, hydroxyl, carboxy, carbamoyl, sulfamoyl and mercapto;
a and b are integers independently selected from 0 to 2;
m and n are integers independently selected from 0 to 2;
Z₁ and Z₂ are independently selected from a hydrophilic substituent group;
C and D are independently selected from aryl, heteroaryl, heterocyclyl, cycloalkyl, cycloalkenyl and a group of the formula:

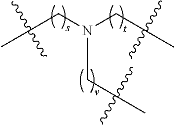

wherein:
s, t and v are integers independently selected from 1 or 2;
[symbol] denotes the point of attachment;
R₃ and R₄ are independently selected from halo, (1-4C)alkyl, (1-4C)alkoxy, amino, nitro, (1-4C)alkylamino, (1-4C)dialkylamino, (1-4C)haloalkyl, (1-4C)haloalkoxy, cyano, (2-4C)alkenyl, (2-4C)alkynyl and a group of the formula:

-L¹-Y¹-Q¹
wherein:
L¹ is absent or a (1-5C)alkylene optionally substituted by one or more substituents selected from (1-2C)alkyl and oxo;
Y¹ is absent or selected from a one of the following groups; O, S, SO, SO₂, N(Rₐ), C(O), C(O)O, OC(O), C(O)N(Rₐ), N(Rₐ)C(O), N(R_{b})C(O)N(Rₐ), N(Rₐ)C(O)O, OC(O)N(Rₐ), S(O)₂N(Rₐ), and N(Rₐ)SO₂, wherein Rₐ and R_{b} are each independently selected from hydrogen and (1-4C)alkyl; and
Q₁ is hydrogen, (1-8C)alkyl, (2-6C)alkenyl, (2-6C)alkynyl, aryl, (3-10C)cycloalkyl, (3-10C)cycloalkenyl, heteroaryl and heterocyclyl; wherein Q₁ is optionally further substituted by one or more substituent groups independently selected from (1-4C)alkyl, halo, (1-4C)haloalkyl, (1-4C)haloalkoxy, amino, (1-4C)aminoalkyl, cyano, hydroxy, carboxy, carbamoyl, sulfamoyl, mercapto, ureido, oxy, NR_{c}R_{d}, OR_{c}, C(O)R_{d}, C(O)OR_{c}, OC(O)R_{c}, C(O)N(R_{d})R_{c}, N(R_{d})C(O)R_{c}, S(O)_{y}R_{c} (where y is 0, 1, or 2), SO₂N(R_{d})R_{c}, N(R_{d})SO₂R_{c}, Si(Rₑ)(R_{d})R_{c} and (CH₂)_{z}NR_{d}R_{c} (where z is 1, 2 or 3); wherein R_{c}, R_{d} and Rₑ are each independently selected from hydrogen, (1-6C)alkyl and (3-6C)cycloalkyl;
and R_{c} and R_{d} can be linked such that, together with the nitrogen atom to which they are attached, they form a 4-7 membered heterocyclic ring which is optionally substituted by one or more substituents selected from (1-4C)alkyl, halo, (1-4C)haloalkyl, (1-4C)haloalkoxy, (1-4C)alkoxy, (1-4C)alkylamino, amino, cyano or hydroxyl; and wherein
two R₃ and/or two R₄ groups taken together may form a group of the formula:

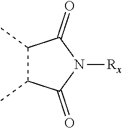

wherein:
Rₓ is selected from hydrogen and (1-6C)alkyl optionally substituted by one or more substituent groups selected from halo, (1-4C)haloalkyl, (1-4C)haloalkoxy, cyano, hydroxy, sulfamoyl, mercapto, ureido, NR_{f}R_{g}, OR_{f}, C(O)R_{f}, C(O)OR_{f}, OC(O)R_{f}, C(O)N(R_{g})R_{f} and N(R_{g})C(O)R_{f}, wherein R_{f} and R_{g} are selected from hydrogen and (1-4C)alkyl; and
the dashed lines represent the points of attachment to C and/or D;
W₁, W₂, W₃ and W₄ are independently selected from CRₕRᵢ, wherein Rₕ and Rᵢ, are selected from hydrogen and (1-2C)alkyl;
X₁, X₂, X₃ and X₄ are independently selected from a group of the formula:

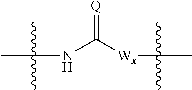

wherein:
   [symbol] denotes the point of attachment;
   W_{X} is selected from O or NH; and
   Q is selected from O, S and NRⱼ, wherein Rⱼ is selected from hydrogen, (1-4C)alkyl, aryl, heteroaryl and sulfonyl;
Z₃ and Z₄ are independently selected from a hydrophilic substituent group;
L is absent or a linker, which optionally bears a hydrophilic substituent group Z₅;
c and d are integers independently selected from 0 to 4; and
and p are integers independently selected from 0 to 2.

In particular embodiments, the compound of Formula I comprises at least one hydrophilic substituent group (e.g. Z₁, Z₂, Z₃, Z₄ or Z₅).

### Macrocycle Tether Group

The saccharide-binding compound of the present invention include one or more macrocycle tether groups. Each macrocycle tether group attaches (and may include) one or more tether functional groups to the macrocycle of the saccharide-binding compound, the macrocycle tether groups being selected from a macrocycle membrane blocking group MMBG, a terminal group T_{MT}, a chromophore group, a semi-solid or solid support, and the fluorescent reporter moiety and cavity-binding moiety.

The macrocycle tether group may be:
(i) A macrocycle membrane blocking group MMBG tether group including a macrocycle membrane blocking group and one or more macrocycle linker units (L_{Munit}) linking the macrocycle of the saccharide-binding compound and the macrocycle membrane blocking group MMBG;
(ii) A terminal tether group including a terminal group T_{MT} and one or more macrocycle linker units (L_{Munit}) linking the macrocycle of the saccharide-binding compound and the terminal group T_{MT};
(iii) A chromophore tether group including a chromophore and one or more macrocycle linker units (L_{Munit}) linking the macrocycle of the saccharide-binding compound and the chromophore;
(iv) A support tether group including one or more macrocycle linker units (L_{Munit}) linking the macrocycle of the saccharide-binding compound and a semi-solid or solid support; or
(v) A cavity-binding tether group including a fluorescent reporter moiety, a cavity-binding moiety and one or more macrocycle linker units (L_{Munit}) linking the macrocycle of the saccharide-binding compound, the fluorescent reporter moiety and cavity-binding moiety.

The or each macrocycle tether group is typically at a position associated with one or more of the substituent groups R₁ₐ, R_{1b}, R₂ₐ, R_{2b}, R₁, R₂, R₃, R₄, R₅, Z₁, Z₂, Z₃, Z₄ and/or Z₅ of formula I described herein. In other words, the saccharide-binding compound may be a compound of formula (I) where one or more of the substituent groups R₁ₐ, R_{1b}, R₂ₐ, R_{2b}, R₁, R₂, R₃, R₄, R₅, Z₁, Z₂, Z₃, Z₄ and/or Z₅ is replaced with a macrocycle tether group as described herein.

### Macrocycle Membrane Blocking Group (MMBG)

Where the macrocycle tether group includes a macrocycle membrane blocking group (MMBG), the system may include a molecular weight cut off membrane. In these embodiments, the macrocycle membrane blocking group MMBG has a molecular weight sufficient to increase the molecular weight of the saccharide-binding compound above the molecular weight cut off of the molecule weight cut off membrane.

In some embodiments, the macrocycle membrane blocking group MMBG has a molecular weight sufficient to increase the molecular weight of the saccharide-binding compound above about 1,000 daltons (1 kDa). The macrocycle membrane blocking group MMBG may have a molecular weight sufficient to increase the molecular weight of the saccharide-binding compound above about 2,000 daltons (2 kDa), about 5,000 daltons (5 kDa), about 10,000 daltons (10 kDa) or about 30,000 daltons (kDa).

The macrocycle membrane blocking group MMBG may have a molecular weight of at least about 1,000 daltons (1 kDa), at least about 2,000 daltons (2 kDa), at least about 5,000 daltons (5 kDa), at least about 10,000 daltons (10 kDa) or at least about 30,000 daltons (kDa).

The macrocycle membrane blocking group MMBG may be a water soluble polymer, such as water soluble polysaccharides (such as dextran), polyacrylamides, polyethylene glycol, peptides (such as oligopeptides or polypeptides) and nucleic acids (such as DNA or RNA).

In particular embodiments, the macrocycle membrane blocking group is a water soluble polysaccharide, polyacrylamide or polyethylene glycol having a molecular weight of at least about 30 kDa. In more particular embodiments, the macrocycle membrane blocking group is a dextran or polyethylene glycol having a molecular weight of at least about 30 kDa. In specific embodiments, the macrocycle membrane blocking group is a dextran of polyethylene glycol having a molecular weight of in the range of about 30 kDa to about 50 kDa, such as about 40 kDa.

### Terminal Group T_{MT}

The terminal group is a monovalent group for terminating the macrocycle tether group (or a branch of the macrocycle tether group). In some embodiments, the terminal group is capable of reacting with a semi-solid or solid support or precursors of a semi-solid or solid support (such as monomers or oligomers). In other embodiments, the terminal group may be a solubilizing group for increasing the solubility of the saccharide-binding compound in a solvent, such as water.

In some embodiments, T_{MT} may be selected from the group consisting of X, OH, SH, SOX, SO₂X, SO₃H, N(R^{x4})H, C(O)H, C(O)OH, OC(O)H, C(O)N(R^{x4})H, N(R^{x4})C(O)H, N(R^{x4})C(O)N(R^{x5})H, N(R^{x4})C(O)OH, OC(O)N(R^{x4})H, S(O)₂N(R^{x4})H, N(R^{x4})SO₂H, —N₃, (1-20C)alkyl, (1-20C)alkyl oxide, (1-20C)alkene and (1-20C)alkyne, aryl, (3-10C)cycloalkyl, (3-10C)cycloalkene, heteroaryl, heterocyclyl, peptides (such as oligopeptides or polypeptides) and nucleic acids (such as DNA or RNA), wherein X is a halogen; and R^{x4} and R^{x5} are each independently selected from hydrogen and (1-4C)alkyl.

In particular embodiments, the terminal group is selected from —C═CH; —CH═CH₂, bicyclo[6.1.0]nonynl, -tBu, —N₃, —NH₂.

### Fluorescent Reporter Moiety

The fluorescent reporter moiety of the cavity-binding tether group may include a fluorophore. In some embodiments, the fluorescent reporter moiety is selected from xanthene dyes or derivatives (such as fluorescein, rhodamine, Oregon green, eosin, and Texas red); cyanine dyes or derivatives (such as cyanine, indocarbocyanine, oxacarbocyanine, thiacarbocyanine, and merocyanine); squaraine dyes or derivatives and ring-substituted squaraines (such as Seta and Square dyes); squaraine rotaxane dyes or derivatives (such as Tau dyes); naphthalene dyes or derivatives (such as dansyl and prodan dyes or derivatives); coumarin dyes or derivatives; oxadiazole dyes or derivatives (such aspyridyloxazole, nitrobenzoxadiazole and benzoxadiazole); anthracene dyes or derivatives (such as anthraquinones, e.g. DRAQ5, DRAQ7 and CyTRAK Orange); pyrene dyes or derivatives (such as cascade blue); oxazine dyes or derivatives (such as Nile red, Nile blue, cresyl violet, oxazine 170); acridine dyes or derivatives (such as proflavin, acridine orange, acridine yellow); arylmethine dyes or derivatives (such as auramine, crystal violet, malachite green); tetrapyrrole dyes or derivatives (such as porphin, phthalocyanine, bilirubin); dipyrromethene dyes or derivatives (such as BODIPY, aza-BODIPY), metal coordination complexes or organometallic fluorophores (such as [Ru(bpy)₃]²⁺ (“Ru-bpy”) and Ln³⁺ chelate complexes), fluorescent dyes in the Alexa Fluor™ series (such as AF488™ and AF594™) and quinoline dyes or derivatives.

With respect to the fluorescent reporter moiety, the term “derivative” as used herein refers to any fluorophore moiety that shares a core structure with the original compound and retains fluorescent activity. For example, xanthene dyes or derivatives are well known dyes containing a xanthylium or di-benzo-g-pyran nucleus as the chromophore with amino or hydroxy group meta to the oxygen.

In particular embodiments, the fluorescent reporter moiety is selected from a xanthene derived dye, a cyanine derived dye, a napthalene derived dye, a pyrene derived dye, a pyranine derived dye, metal coordination complexes or organometallic fluorophores and a fluorescent dye in the Alexa Fluor™ series. Specific examples of the fluorophore include but are not limited to AF430, AF488, AF594, BODIPY, Cyanine, Cy3, Cy7.5, EDANS, Fluorescein, Pyranine, Rhodamine 6G, Rhodamine B, Ru(BIPY)3, TAMRA, Phenazine.

In more particular embodiments, the fluorescent reporter moiety is selected from a xanthene derived dye, such as fluorescein and rhodamine, [Ru(bpy)₃]²+, a Ln³⁺ chelate complex and a fluorescent dye in the Alexa Fluor™ series, such as AF488™ and AF™.

### Displaceable Cavity-Binding Moiety

The cavity-binding moiety of the cavity-binding tether group may be selected so that cavity-binding moiety is displaced from the cavity of the saccharide-binding compound when the glucose sensing system is contacted with the target saccharide-containing aqueous environment.

In some embodiments, the cavity-binding moiety includes a hydrogen-bond acceptor. In particular embodiments, the cavity-binding moiety includes a saccharide, such as a glucoside or a glycoside. The saccharide may be linked to the rest of the cavity-binding substituent group by a beta-linkage. In more particular embodiments, the cavity-binding moiety includes a glucoronide. In specific embodiments, the cavity-binding moiety is salidroside.

### Chromophore

The chromophore may be an energy transfer donor or acceptor, such as a Forster resonance energy transfer (FRET) or photoinduced energy transfer (PET) donor or acceptor. In some embodiments, the chromophore of the chromophore tether group and the fluorescent reporter moiety form an energy transfer donor/acceptor pair FRET or PET donor/acceptor pair.

In some embodiments, the chromophore is a fluorophore selected from xanthene dyes or derivatives (such as fluorescein, rhodamine, Oregon green, eosin, and Texas red); cyanine dyes or derivatives (such as cyanine, indocarbocyanine, oxacarbocyanine, thiacarbocyanine, and merocyanine); squaraine dyes or derivatives and ring-substituted squaraines (such as Seta and Square dyes); squaraine rotaxane dyes or derivatives (such as Tau dyes); naphthalene dyes or derivatives (such as dansyl and prodan dyes or derivatives); coumarin dyes or derivatives; oxadiazole dyes or derivatives (such aspyridyloxazole, nitrobenzoxadiazole and benzoxadiazole); anthracene dyes or derivatives (such as anthraquinones, e.g. DRAQ5, DRAQ7 and CyTRAK Orange); pyrene dyes or derivatives (such as cascade blue and sulphonated pyrenes); oxazine dyes or derivatives (such as Nile red, Nile blue, cresyl violet, oxazine 170); acridine dyes or derivatives (such as proflavin, acridine orange, acridine yellow); arylmethine dyes or derivatives (such as auramine, crystal violet, malachite green); tetrapyrrole dyes or derivatives (such as porphin, phthalocyanine, bilirubin); and dipyrromethene dyes or derivatives (such as BODIPY, aza-BODIPY), metal coordination complexes or organometallic fluorophores (such as [Ru(bpy)₃]²+(“Ru-bpy”) and Ln³⁺ chelate complexes), fluorescent dyes in the Alexa Fluor™ series (such as AF488™ and AF594™) and quinoline dyes or derivatives.

Macrocycle linker units L_{Munit}

The macrocycle tether groups typically include one or more macrocycle linker units (L_{Munit}) linking the macrocycle of the saccharide-binding compound to one or more of the tether functional groups. In some embodiments, the macrocycle linker units may be units formed from a “click” reaction.

In some embodiments, the macrocycle of the saccharide-binding compound is linked by the macrocycle linker unit(s) to only one of the macrocycle tether functional groups or the semi-solid or solid support. In these embodiments, the macrocycle linker units (L_{Munit}) may consist of one or more linear linkers independently selected from the group consisting of O, S, SO, SO₂, N(R^{x4}), C(O), C(O)O, OC(O), C(O)N(R^{x4}), N(R^{x4})C(O), N(R^{x4})C(O)N(R^{x5}), N(R^{x4})C(O)O, OC(O)N(R^{x4}), S(O)₂N(R^{x4}), N(R^{x4})SO₂, amino acids, (1-20C)alkylene, (1-20C)alkylene oxide, (1-20C)alkenyl, (1-20C)alkynyl, aryl, (3-10C)cycloalkyl, (3-10C)cycloalkenyl, heteroaryl and heterocyclyl, wherein R^{x4} and R^{x5} are each independently selected from hydrogen and (1-4C)alkyl.

In these embodiments, the or each macrocycle tether group may include from 1 to 25 linear linker units and each linear linker unit is independently selected from the group consisting of 0, N(R^{x4}), C(O), C(O)O, C(O)N(R^{x4}), N(R^{x4})C(O)N(R^{x5}), (O1-4Calkyl)_{g}, C(O)1-4Calkyl, N(R^{x4})1-4C alkyl, C(O)N(R^{x4})1-4C alkyl, N(R^{x4})C(O)1-4C alkyl, N(R^{x4})C(O)N(R^{x4})1-4C alkyl, -(amino acid)-, triazolyl-1-4Calkyl, 1-4Calkyl-triazolyl-1-4Calkyl, wherein g is an integer selected from 1 to 200, j is an integer selected from 1 to 200 and R^{x4} and R^{x5} are each independently selected from hydrogen and (1-4C)alkyl. In some embodiments, g is an integer from 1 to 50, 1 to 25, 1 to 20, 1 to 15 or 1 to 5. In some embodiments, g is an integer from 2 to 50, 2 to 25, 2 to 20, 2 to 15 or 2 to 5. In some embodiments, j is an integer from 100 to 200, from 125 to 175 or from 140 to 160.

The cavity-binding tether group may include two sets of linkers, a first set of linker units (L_{CB1}) linking the macrocycle to one of the fluorescent reporter moiety or the displaceable cavity-binding moiety and a second set of linker units (L_{CB2}) linking the fluorescent reporter moiety or the displaceable cavity-binding moiety to the displaceable cavity-binding moiety or the fluorescent reporter moiety, respectively. L_{CB1}and L_{CB2} are typically one or more linear linker units as described herein. In other words, the cavity-binding tether group may be represented as:
M-L_{CB1}-fluorescent reporter moiety-L_{CB2}-displaceable cavity binding moiety; or
M-L_{CB1}-displaceable cavity binding moiety-L_{CB2}-fluorescent reporter moiety Where M is the macrocycle.

LCB₁ and/or LCB₂ may include linear linkers as defined herein. In alternative embodiments, the cavity-binding tether group includes linker units linking the macrocycle to the displaceable cavity-binding moiety or the fluorescent reporter moiety and the other of the displaceable cavity-binding moiety or the fluorescent reporter moiety is either substituted onto one of the linker units or one or more of the linker units is replaced with a branched linker, wherein each branched linker is a linear linker as described herein substituted with one or more linear linkers as described herein and linking the branched linker to one or more of the linear linkers is replaced with a branched linker, wherein each branched linker is a linear linker as described herein substituted with one or more linear linkers as described herein and linking the branched linker to one of the functional groups.

For example, the cavity-binding tether group may include one or more linear linker units from the macrocycle up to a linker unit substituted with one of the displaceable cavity-binding moiety or the fluorescent reporter moiety and then further linear linker units from the substituted linker unit to the other of the displaceable cavity-binding moiety or the fluorescent reporter moiety or a branched linker unit. Alternatively, the cavity-binding tether group may include one or more linear linker units from the macrocycle up to a branched linker unit, the branched linker unit being further attached to two sets of one or more linker units, each set of linker units linking the branched linker unit to one of the displaceable cavity-binding moiety and the fluorescent reporter moiety. In other words, one set of linker units is attached to the branched linker unit and the displaceable cavity-binding moiety and the other set of linker units is attached to the branched linker unit and the fluorescent reporter moiety.

In some embodiments, the macrcocyle tether group include one or more further macrocycle tether functional groups. In particular:
a macrocycle membrane blocking group MMBG tether group may further include one or more of a further macrocycle membrane blocking groups MMBG, a terminal group T_{MT}, and/or a chromophore;
A terminal tether group may further include a macrocycle membrane blocking group MMBG, a further terminal group T_{MT} and/or a chromophore;
A chromophore tether group may further include a macrocycle membrane blocking group MMBG, a terminal group T_{MT} and/or a further chromophore; and
A support tether group may further include a terminal group T_{MT} and/or a chromophore;
A cavity-binding tether group may further include a macrocycle membrane blocking group MMBG, a terminal group T_{MT} and/or a chromophore.

It will be appreciated that, for example, a macrocycle membrane blocking group tether group with a macrocycle membrane blocking group and a terminal group can also be considered as a terminal tether group with a macrocycle membrane blocking group.

In these embodiments, the macrocycle tether group may include one or more of the linear linkers described herein and one or more of the linear linkers is either substituted with one of the macrocycle tether functional groups or one or more of the linear linkers is replaced with a branched linker, wherein each branched linker is a linear linker as described herein substituted with one or more linear linkers as described herein and linking the branched linker to one of the functional groups.

In particular embodiments, the saccharide-binding compound includes a macrocycle membrane blocking group MMBG tether group including a macrocycle membrane blocking group and one or more macrocycle linker units (L_{Munit}) linking the macrocycle of the saccharide-binding compound and the macrocycle membrane blocking group MMBG, and further includes a terminal group T_{MT}.

In particular embodiments, the or each macrocycle tether group has the structure selected from the group consisting of:

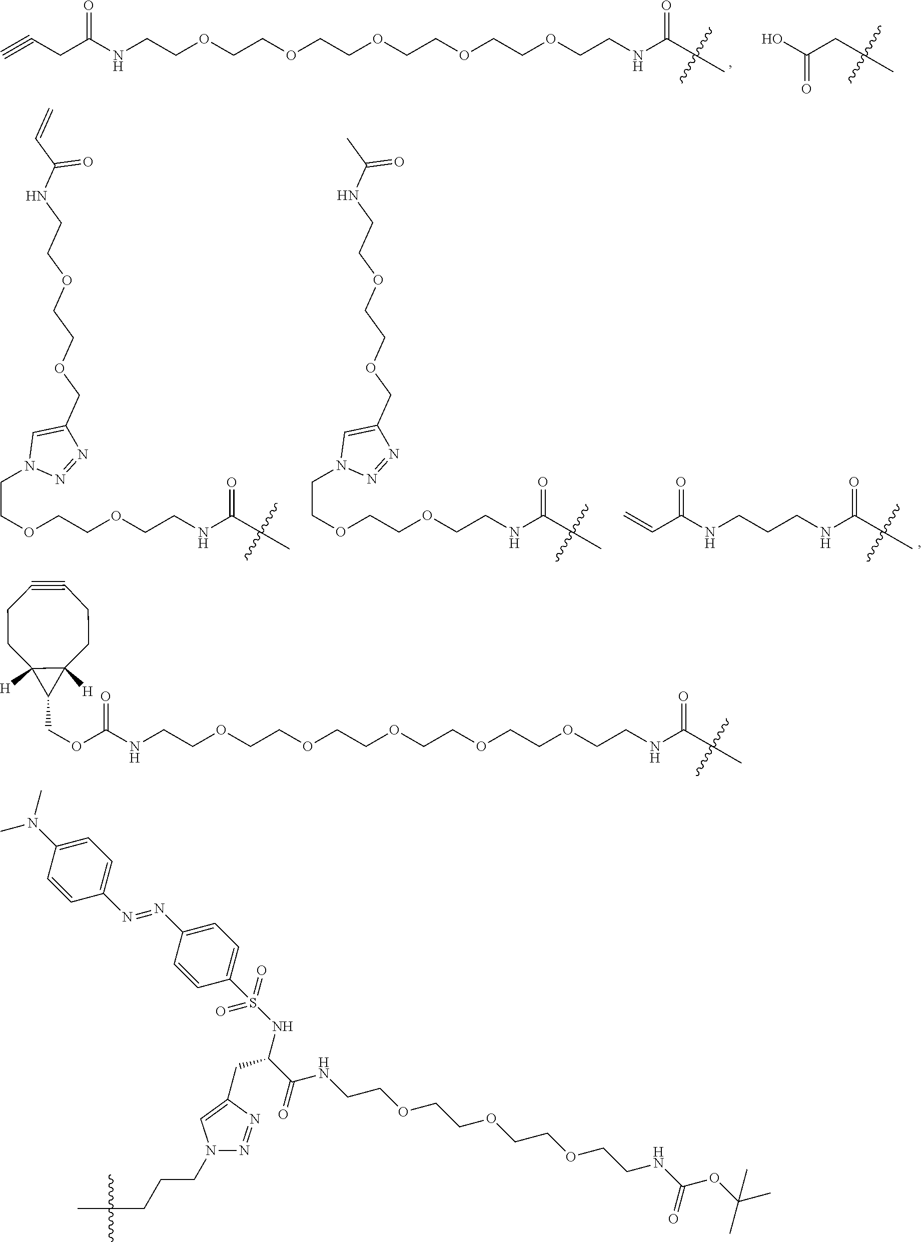

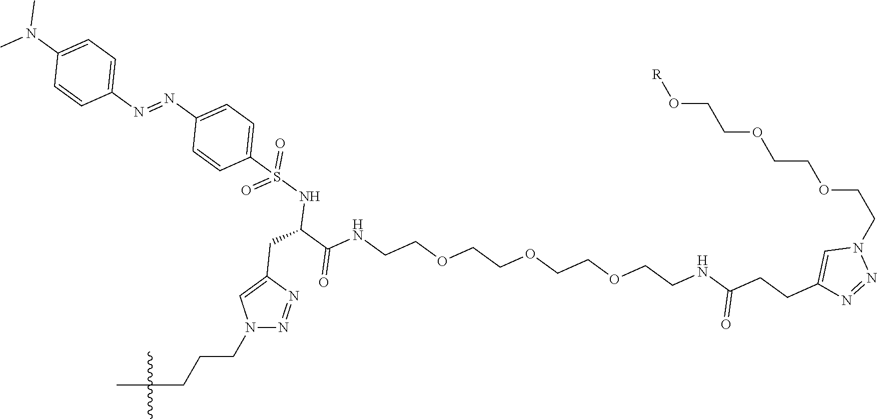

where R is 40 kDa PEG,

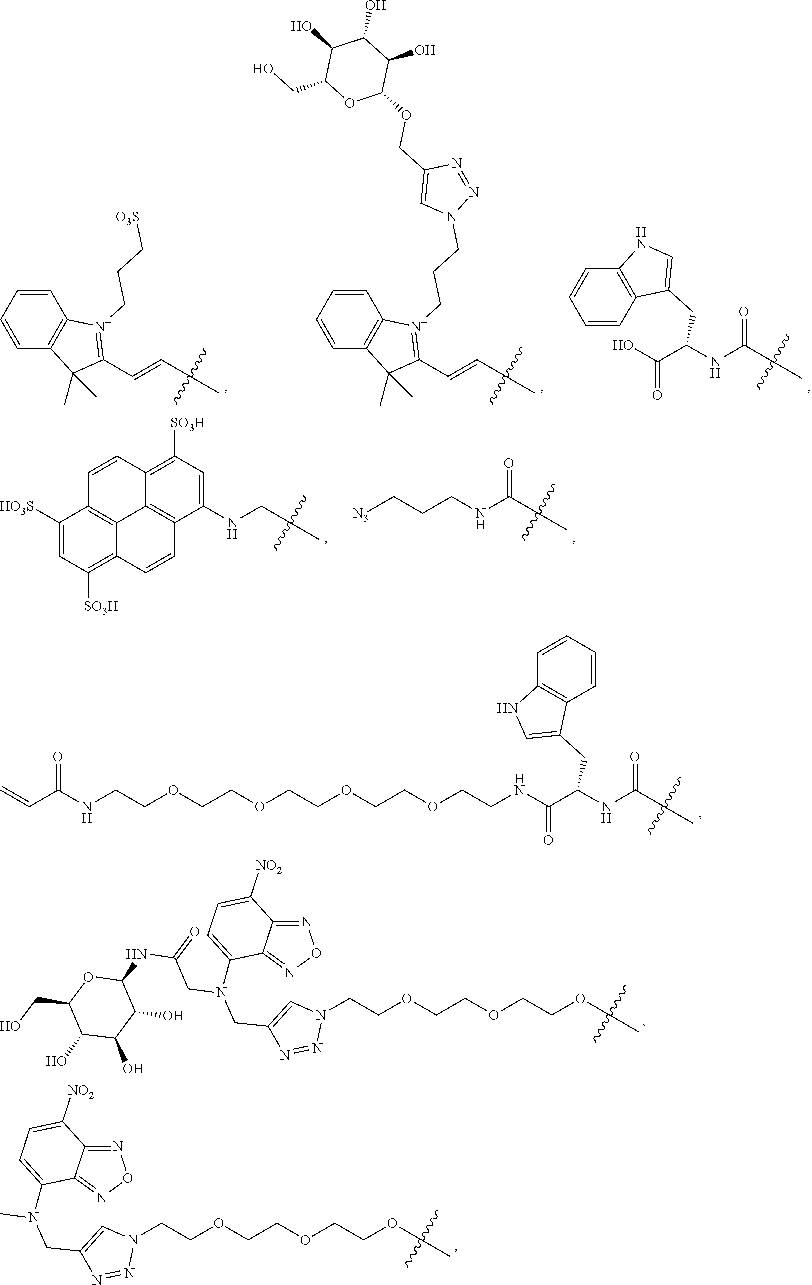

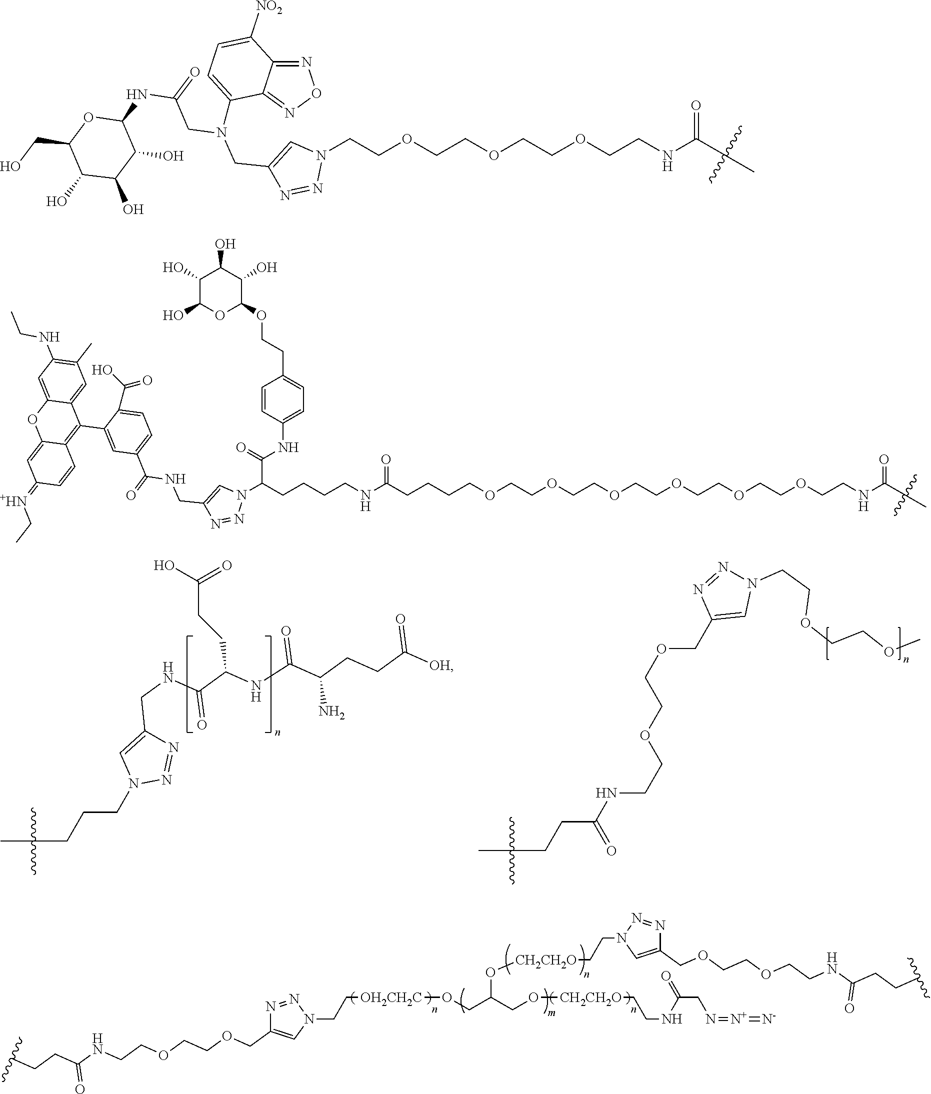

(where m is about 4 to 8, e.g. 6 and n is between about 100 and about 120 (e.g. about 110, 111 or 112)

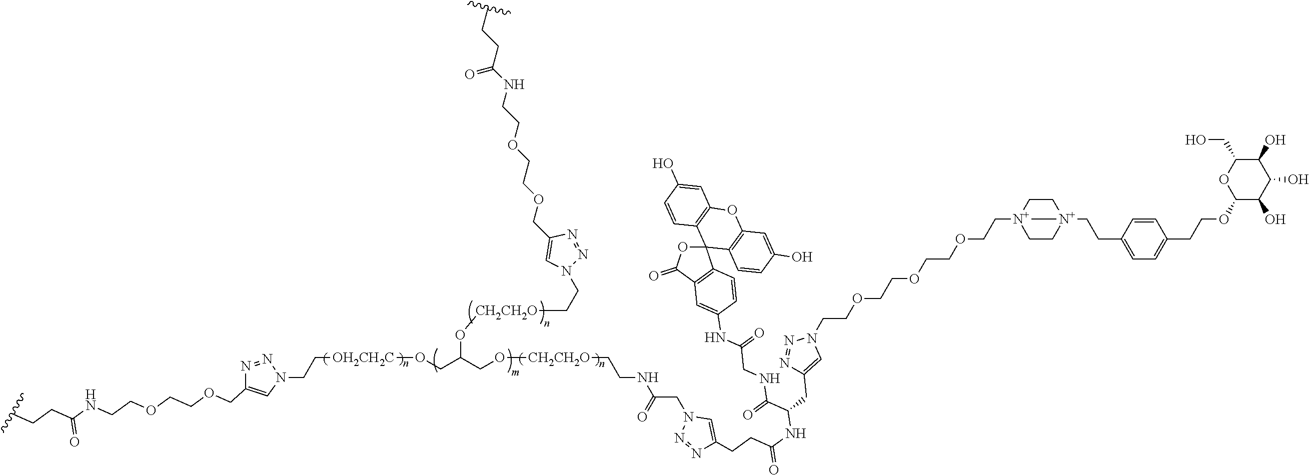

(where m is about 4 to 8, e.g. 6 and n is between about 100 and about 120 (e.g. about 110, 111 or 112)

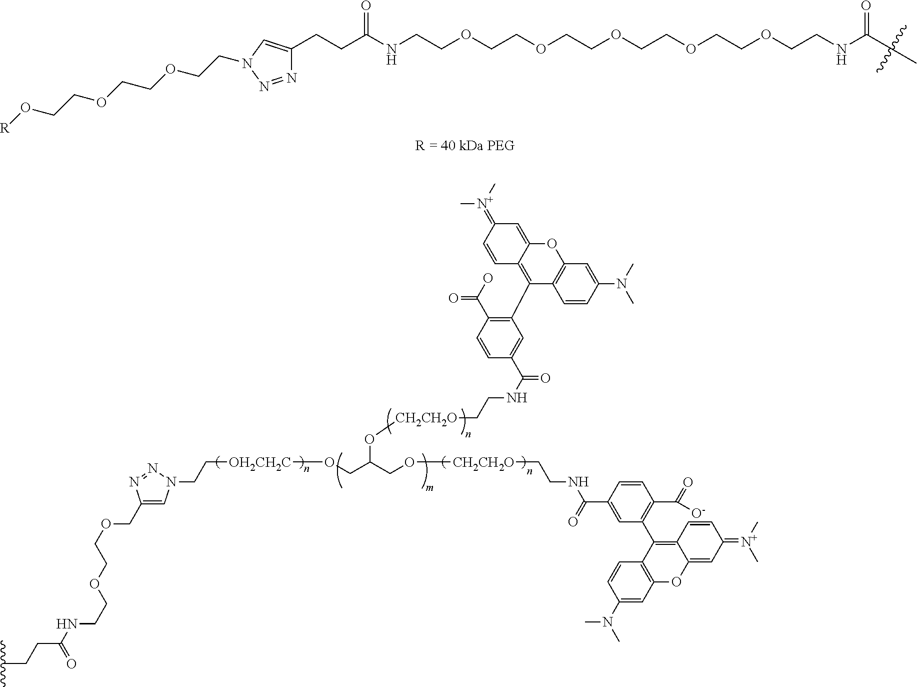

(where m is about 4 to 8, e.g. 6 and n is between about 100 and about 120 (e.g. about 110, 111 or 112)

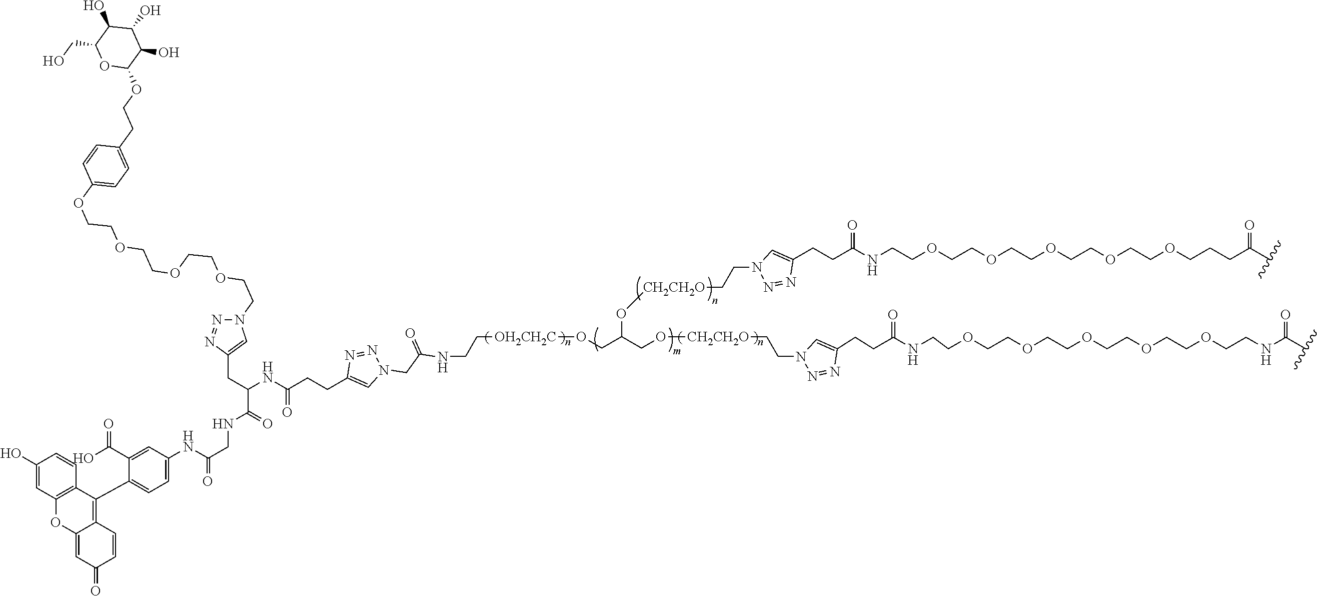

(where m is about 4 to 8, e.g. 6 and n is between about 100 and about 120 (e.g. about 110, 111 or 112)

Wherein [symbol] denotes the point of attachment to the macrocycle.

The or each macrocycle tether group may be attached to the macrocycle in a position 10 associated with R₁ₐ, R_{1b}, R₂ₐ, R_{2b}, R₁, R₂, R₅, Z₁, Z₂, and/or Z₅. These positions are sometimes referred to as being attached to the “pillars” of the macrocycle (represented by the groups extending vertically in formula (I)). In alternative embodiments, The or each macrocycle tether group may be attached to the macrocycle in a position associated with R₃, R₄, Z₃, and/or Z₄. These positions are sometimes referred to as being attached to the “roof” or “floor” of the macrocycle (as represented by rings C and D in formula I).

### Further Embodiments of the Saccharide-Binding Compound

In particular embodiments, the saccharide-binding compound each of bonds b₁ and b₂, Rings A and B, C, D, R₁, R₂, R₃, R₄, W₁, W₂, W₃, W₄, X₁, X₂, X₃, X₄, Z₁, Z₂, Z₃, Z₄, Z₅, L, a, b, c, d, m, n, o, p and any associated substituent groups has any of the meanings defined hereinbefore or in any of paragraphs (1) to (60) hereinafter. The preferred definitions of the saccharide-binding compounds below are to be in combination with the option that the saccharide-binding compound includes one or more macrocycle tether groups as defined herein. In other words, all preferred embodiments of the saccharide-binding compound below may include one or more macrocycle tether groups as defined herein.
(1) bonds b₁ and b₂ are single bonds;
(2) bonds b₁ and b₂ are double bonds;
(3) R₁ₐ, R_{1b}, R₂ₐ and R_{2b} are independently selected from (1-8C)alkyl, (3-10C)cycloalkyl, aryl, heteroaryl and heterocyclyl, each of which is optionally substituted by one or more substituent groups selected from (1-4C)alkyl, halo, (1-4C)haloalkyl, (1-4C)haloalkoxy, (1-4C)alkoxy, amino, cyano, hydroxyl and a hydrophilic substituent group; or
R₁ₐ and R_{1b} are linked so as to form a group of the formula:

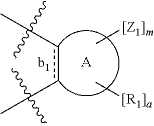

and/or R₂ₐ and R_{2b} are linked so as to form a group of the formula:

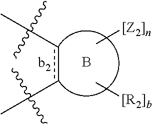

wherein:
[symbol] denotes the point of attachment;
bonds bi and b2 are as described above;
Rings A and B are independently selected from aryl, heteroaryl, heterocyclyl, cycloalkyl and cycloalkenyl;
R₁ and R₂ are independently selected from (1-4C)alkyl, halo, (1-4C)haloalkyl, (1-4C)haloalkoxy, (1-4C)alkoxy, (1-4C)alkylamino, amino, cyano, hydroxyl, carboxy, carbamoyl, sulfamoyl and mercapto;
a and b are integers independently selected from 0 to 2;
m and n are integers independently selected from 0 to 2;
Z₁ and Z₂ are independently selected from hydrophilic substituent groups;
(4) R₁ₐ, R_{1b}, R₂ₐ and R_{2b} are independently selected from aryl and heteroaryl, each of which is optionally substituted by one or more substituent groups selected from (1-4C)alkyl, halo, (1-4C)alkoxy, amino or hydroxyl; or
R₁ₐ and R_{1b} are linked so as to form a group of the formula:

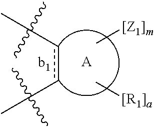

and/or R₂ₐ and R_{2b} are linked so as to form a group of the formula:

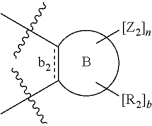

wherein:
[symbol] denotes the point of attachment;
bonds b₁ and b2 are as described above;
Rings A and B are independently selected from aryl, heteroaryl, heterocyclyl, cycloalkyl and cycloalkenyl;
R₁ and R₂ are independently selected from (1-4C)alkyl, halo, (1-4C)haloalkyl, (1-4C)haloalkoxy, (1-4C)alkoxy, (1-4C)alkylamino, amino, cyano, hydroxyl, carboxy, carbamoyl, sulfamoyl and mercapto;
a and b are integers independently selected from 0 to 2;
m and n are integers independently selected from 0 to 2;
Z₁ and Z₂ are independently selected from hydrophilic substituent groups;
(5) R₁ₐ and R_{1b} are linked so as to form a group of the formula:

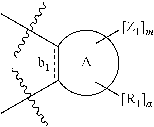

and R₂ₐ and R_{2b} are linked so as to form a group of the formula:

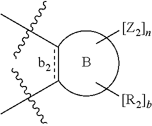

wherein:
[symbol] denotes the point of attachment;
bonds bi and b2 are as described above;
Rings A and B are independently selected from aryl, heteroaryl, heterocyclyl, cycloalkyl or cycloalkenyl;
R₁ and R₂ are independently selected from (1-4C)alkyl, halo, (1-4C)haloalkyl, (1-4C)haloalkoxy, (1-4C)alkoxy, (1-4C)alkylamino, amino, cyano, hydroxyl, carboxy, carbamoyl, sulfamoyl and mercapto;
a and b are integers independently selected from 0 to 2;
m and n are integers independently selected from 0 to 2;
Z₁ and Z₂ are independently selected from hydrophilic substituent groups; (6) Rings A and B are independently selected from aryl, heteroaryl and heterocyclyl (e.g. pyrrolidinyl);
(7) Rings A and B are independently selected from aryl and heteroaryl;
(8) Rings A and B are aryl;
(9) Rings A and B are independently selected from phenyl, pyridyl, naphthyl, and pyrrolidinyl;
(10) Rings A and B are phenyl or pyrrolidinyl, preferably phenyl;
(11) R₁ and R₂ are independently selected from (1-4C)alkyl, halo, (1-4C)haloalkyl, (1-4C)haloalkoxy, (1-4C)alkoxy, (1-4C)alkylamino, amino, cyano and hydroxyl;
(12) R₁ and R₂ are independently selected from (1-4C)alkyl, halo, amino, cyano and hydroxyl;
(13) C and D are independently selected from aryl, heteroaryl, heterocyclyl, cycloalkyl, cycloalkenyl and a group of the formula:

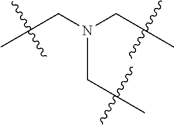

wherein [symbol] denotes the point of attachment;
(14) C and D are independently selected from aryl, heteroaryl, heterocyclyl, cycloalkyl, cycloalkenyl;
(15) C and D are independently selected from aryl and heteroaryl;
(16) C and D are independently selected from phenyl, naphthenyl and anthracenyl;
(17) C and D are phenyl;
(18) C and D are anthracenyl;
(19) R₃ and R₄ are independently selected from halo, (1-4C)alkyl, (1-4C)alkoxy, amino, nitro, (1-4C)alkylamino, (1-4C)dialkylamino, (1-4C)haloalkyl, (1-4C)haloalkoxy, cyano, (2-4C)alkenyl, (2-4C)alkynyl and a group of the formula:

-L¹-Y¹-Q¹
wherein:
L¹ is absent or (1-5C)alkylene;
Y¹ is absent or selected from a one of the following groups; O, S, SO, SO₂, N(Rₐ), C(O), C(O)O, OC(O), C(O)N(Rₐ), N(Rₐ)C(O), N(R_{b})C(O)N(Rₐ), N(Rₐ)C(O)O, OC(O)N(Rₐ), S(O)₂N(Rₐ), and N(Rₐ)SO₂, wherein Rₐ and R_{b} are each independently selected from hydrogen and (1-4C)alkyl; and
Q₁ is hydrogen, (1-8C)alkyl, (2-6C)alkenyl, (2-6C)alkynyl, aryl, (3-10C)cycloalkyl, (3-10C)cycloalkenyl, heteroaryl and heterocyclyl; wherein Q₁ is optionally further substituted by one or more substituent groups independently selected from (1-4C)alkyl, halo, (1-4C)haloalkyl, (1-4C)haloalkoxy, amino, (1-4C)aminoalkyl, cyano, hydroxy, carboxy, carbamoyl, sulfamoyl, mercapto, ureido, oxy, NR_{c}R_{d}, OR_{c}, C(O)R_{d}, C(O)OR_{c}, OC(O)R_{c}, C(O)N(R_{d})R_{c}, N(R_{d})C(O)R_{c}, S(O)_{y}R_{c} (where y is 0, 1 or 2), SO₂N(R_{d})R_{c}, N(R_{d})SO₂R_{c}, Si(Rₑ)(R_{d})R_{c} and (CH₂)_{z}NR_{d}R_{c} (where z is 1, 2 or 3); wherein R_{c}, R_{d} and Rₑ are each independently selected from hydrogen, (1-6C)alkyl or (3-6C)cycloalkyl; and
two R₃ and/or two R₄ groups taken together may form a group of the formula:

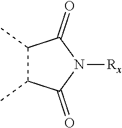

wherein:
Rₓ is selected from hydrogen and (1-6C)alkyl optionally substituted by one or more substituent groups selected from halo(1-4C)haloalkyl, (1-4C)haloalkoxy, cyano, hydroxy, sulfamoyl, mercapto, ureido, NR_{f}Rₐ, ORₜ, C(O)R_{f}, C(O)OR_{f}, OC(O)R_{f}, C(O)N(R_{g})R_{f} and N(R_{g})C(O)R_{f}, wherein R_{f} and R_{g} are selected from hydrogen and (1-4C)alkyl; and
dashed lines represent the points of attachment to C and/or D;
(20) R₃ and R₄ are independently selected from halo, (1-4C)alkyl, (1-4C)alkoxy, amino, nitro, (1-4C)alkylamino, (1-4C)dialkylamino, (1-4C)haloalkyl, (1-4C)haloalkoxy, cyano, (2-4C)alkenyl, (2-4C)alkynyl and a group of the formula:

-L¹-Y¹-Q¹
wherein:
L¹ is absent or a (1-5C)alkylene;
Y¹ is absent or selected from a one of the following groups; O, S, SO, SO₂, N(Rₐ), C(O), C(O)O, OC(O), C(O)N(Rₐ) and N(Rₐ)C(O), wherein Rₐ is selected from hydrogen and (1-4C)alkyl; and
Q¹ is hydrogen, (1-8C)alkyl, (2-6C)alkenyl, (2-6C)alkynyl, aryl, (3-10C)cycloalkyl, (3-10C)cycloalkenyl, heteroaryl and heterocyclyl; wherein Q¹ is optionally further substituted by one or more substituent groups independently selected from (1-4C)alkyl, halo, (1-4C)haloalkyl, (1-4C)haloalkoxy, amino, (1-4C)aminoalkyl, cyano, hydroxy, NR_{c}R_{d}, OR_{c}, C(O)R_{d}, C(O)OR_{c}, OC(O)R_{c}, C(O)N(R_{d})R_{c} and N(R_{d})C(O)R_{c}; wherein R_{c} and R_{d} are each independently selected from hydrogen and (1-6C)alkyl;
and two R₃ and/or two R₄ groups taken together may form a group of the formula:

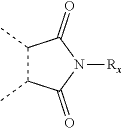

wherein:
Rₓ is selected from hydrogen and (1-6C)alkyl optionally substituted by one or more substituent groups selected from halo, (1-4C)haloalkyl, (1-4C)haloalkoxy, cyano, hydroxy, sulfamoyl, mercapto, ureido, NR_{f}R_{g}, OR_{f}, C(O)R_{f}, C(O)OR_{f}, OC(O)R_{f}, C(O)N(R_{g})R_{f} and N(R_{g})C(O)R_{f}, wherein R_{f} and R_{g} are selected from hydrogen and (1-4C)alkyl; and
dashed lines represent the points of attachment to C and/or D;
(21) R₃ and R₄ are independently selected from halo, (1-4C)alkyl, (1-4C)alkoxy, amino, nitro, (1-4C)alkylamino, (1-4C)dialkylamino, (1-4C)haloalkyl, (1-4C)haloalkoxy, cyano, (2-4C)alkenyl, (2-4C)alkynyl and a group of the formula:

-L¹-Y¹-Q¹
wherein:
L¹ is absent or (1-5C)alkylene;
Y¹ is absent or selected from a one of the following groups; O, N(Rₐ), C(O), C(O)O, OC(O), C(O)N(Rₐ) and N(Rₐ)C(O), wherein Rₐ is selected from hydrogen and (1-4C)alkyl; and
Q¹ is hydrogen, (1-8C)alkyl, aryl, (3-10C)cycloalkyl, (3-10C)cycloalkenyl, heteroaryl and heterocyclyl; wherein Q¹ is optionally further substituted by one or more substituent groups independently selected from (1-4C)alkyl, halo, (1-4C)haloalkyl, (1-4C)haloalkoxy, amino, (1-4C)aminoalkyl, cyano, hydroxy, NR_{c}R_{d}, OR_{c}, C(O)R_{d}, C(O)OR_{c}, OC(O)R_{c}, C(O)N(R_{d})R_{c} and N(R_{d})C(O)R_{c}; wherein R_{c} and R_{d} are each independently selected from hydrogen and (1-6C)alkyl; and
two R₃ and/or two R₄ groups taken together may form a group of the formula:

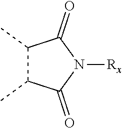

wherein:
Rₓ is selected from hydrogen and (1-6C)alkyl optionally substituted by one or more substituent groups selected from halo, (1-4C)haloalkyl, NR_{f}R_{g}, OR_{f}, C(O)R_{f}, C(O)OR_{f} and C(O)N(R_{g})R_{f}, wherein R_{f} and R_{g} are selected from hydrogen and (1-4C)alkyl; and dashed lines represent the points of attachment to C and/or D;
(22) R₃ and R₄ are independently selected from halo, (1-4C)alkyl, (1-4C)alkoxy, amino, nitro, (1-4C)alkylamino, (1-4C)dialkylamino, (1-4C)haloalkyl, (1-4C)haloalkoxy, cyano, (2-4C)alkenyl, (2-4C)alkynyl and a group of the formula:

-L¹-Y¹-Q¹
wherein:
L¹ is absent or a (1-5C)alkylene optionally substituted by one or more substituents selected from (1-2C)alkyl and oxo;
Y¹ is absent or selected from a one of the following groups; O, S, SO, SO₂, N(Rₐ), C(O), C(O)O, OC(O), C(O)N(Rₐ), N(Rₐ)C(O), N(R_{b})C(O)N(Rₐ), N(Rₐ)C(O)O, OC(O)N(Rₐ), S(O)₂N(Rₐ), and N(Rₐ)SO₂, wherein Rₐ and R_{b} are each independently selected from hydrogen and (1-4C)alkyl; and
Q¹ is hydrogen, (1-8C)alkyl, (2-6C)alkenyl, (2-6C)alkynyl, aryl, (3-10C)cycloalkyl, (3-10C)cycloalkenyl, heteroaryl and heterocyclyl; wherein Q¹ is optionally further substituted by one or more substituent groups independently selected from (1-4C)alkyl, halo, (1-4C)haloalkyl, (1-4C)haloalkoxy, amino, (1-4C)aminoalkyl, cyano, hydroxy, carboxy, carbamoyl, sulfamoyl, mercapto, ureido, oxy, NR_{c}R_{d}, OR_{c}, C(O)R_{d}, C(O)OR_{c}, OC(O)R_{c}, C(O)N(R_{d})R_{c}, N(R_{d})C(O)R_{c}, S(O)_{y}R_{c} (where y is 0, 1 or 2), SO₂N(R_{d})R_{c}, N(R_{d})SO₂R_{c}, Si(Rₑ)(R_{d})R_{c} and (CH₂)_{z}NR_{d}R_{c} (where z is 1, 2 or 3); wherein R_{c}, R_{d} and Rₑ are each independently selected from hydrogen, (1-6C)alkyl and (3-6C)cycloalkyl;
and R_{c} and R_{d} can be linked such that, together with the nitrogen atom to which they are attached, they form a 4-7 membered heterocyclic ring which is optionally substituted by one or more substituents selected from (1-4C)alkyl, halo, (1-4C)haloalkyl, (1-4C)haloalkoxy, (1-4C)alkoxy, (1-4C)alkylamino, amino, cyano and hydroxyl;
(23) R₃ and R₄ are independently selected from halo, (1-4C)alkyl, (1-4C)alkoxy, amino, nitro, (1-4C)alkylamino, (1-4C)dialkylamino, (1-4C)haloalkyl, (1-4C)haloalkoxy, cyano, (2-4C)alkenyl, (2-4C)alkynyl and a group of the formula:

-L¹-Y¹-Q¹
wherein:
L¹ is absent or (1-5C)alkylene;
Y¹ is absent or selected from a one of the following groups; O, S, SO, SO₂, N(Rₐ), C(O), C(O)O, OC(O), C(O)N(Rₐ) and N(Rₐ)C(O), wherein Rₐ is selected from hydrogen and (1-4C)alkyl; and
Q¹ is hydrogen, (1-8C)alkyl, (2-6C)alkenyl, (2-6C)alkynyl, aryl, (3-10C)cycloalkyl, (3-10C)cycloalkenyl, heteroaryl and heterocyclyl; wherein Q¹ is optionally further substituted by one or more substituent groups independently selected from (1-4C)alkyl, halo, (1-4C)haloalkyl, (1-4C)haloalkoxy, amino, (1-4C)aminoalkyl, cyano, hydroxy, carboxy, carbamoyl, sulfamoyl, mercapto, ureido, oxy, NR_{c}R_{d}, OR_{c}, C(O)R_{d}, C(O)OR_{c}, OC(O)R_{c}, C(O)N(R_{d})R_{c}, N(R_{d})C(O)R_{c}, S(O)_{y}R_{c} (where y is 0, 1 or 2), SO₂N(R_{d})R_{c}, N(R_{d})SO₂R_{c}, Si(Rₑ)(R_{d})R_{c} and (CH₂)_{z}NR_{d}R_{c} (where z is 1, 2 or 3); wherein R_{c}, R_{d} and Rₑ are each independently selected from hydrogen, (1-6C)alkyl and (3-6C)cycloalkyl;
(24) R₃ and R₄ are independently selected from halo, (1-4C)alkyl, (1-4C)alkoxy, amino, nitro, (1-4C)haloalkyl, (1-4C)haloalkoxy, cyano and a group of the formula:

-L¹-Y¹-Q¹
wherein:
L¹ is absent or (1-2C)alkylene;
Y¹ is absent or selected from a one of the following groups; O, N(Rₐ), C(O), C(O)O, OC(O), C(O)N(Rₐ) and N(Rₐ)C(O), wherein Rₐ is selected from hydrogen and (1-4C)alkyl; and
Q¹ is hydrogen, (1-8C)alkyl, aryl, (3-10C)cycloalkyl, heteroaryl and heterocyclyl; wherein Q¹ is optionally further substituted by one or more substituent groups independently selected from (1-4C)alkyl, halo, (1-4C)haloalkyl, (1-4C)haloalkoxy, amino, (1-4C)aminoalkyl, cyano, hydroxy, NR_{c}R_{d}, OR_{c}, C(O)R_{d}, C(O)OR_{c}, OC(O)R_{c}, C(O)N(R_{d})R_{c} and N(R_{d})C(O)R_{c}; wherein R_{c} and R_{d} are each independently selected from hydrogen and (1-6C)alkyl;
(25) R₃ and R₄ are independently selected from halo, (1-4C)alkyl, (1-4C)alkoxy, amino, nitro, (1-4C)haloalkyl, (1-4C)haloalkoxy, cyano and a group of the formula:

-L1-Y1-Q¹
wherein:
L¹ is absent or (1-2C)alkylene;
Y¹ is absent or selected from a one of the following groups; O, N(Rₐ), C(O)O and C(O)N(Rₐ), wherein Rₐ is selected from hydrogen and (1-4C)alkyl; and
Q¹ is hydrogen, (1-8C)alkyl, aryl and heteroaryl; wherein Qⁱ is optionally further substituted by one or more substituent groups independently selected from (1-4C)alkyl, halo, (1-4C)haloalkyl, (1-4C)haloalkoxy, amino, (1-4C)aminoalkyl, cyano, hydroxy, NR_{c}R_{d}, OR_{c}, C(O)R_{d}, C(O)OR_{c}, OC(O)R_{c}, C(O)N(R_{d})R_{c} and N(R_{d})C(O)R_{c}; wherein R_{c} and R_{d} are each independently selected from hydrogen and (1-6C)alkyl;
(26) R₃ and R₄ are independently selected from halo, (1-4C)alkyl, (1-4C)alkoxy, amino, nitro, (1-4C)haloalkyl, (1-4C)haloalkoxy, cyano and a group of the formula:

-L¹-Y¹-Q¹
wherein:
L¹ is absent or (1-2C)alkylene;
Y¹ is absent or selected from a one of the following groups; O, N(Rₐ), C(O)O and C(O)N(Rₐ), wherein Rₐ is selected from hydrogen and (1-4C)alkyl; and
Q¹ is hydrogen or (1-8C)alkyl; wherein said (1-8C)alkyl is optionally further substituted by one or more substituent groups independently selected from halo, amino, (1-4C)aminoalkyl, hydroxy, NR_{c}R_{d}, OR_{c}, C(O)R_{d}, C(O)OR and C(O)N(R_{d})R_{c}; wherein R_{c} and R_{d} are each independently selected from hydrogen and (1-2C)alky;
(27) R₃ and R₄ are independently selected from halo, (1-4C)alkyl, (1-4C)alkoxy, amino, nitro, (1-4C)haloalkyl, (1-4C)haloalkoxy and cyano;
(28) R₃ and R₄ are independently selected from (1-4C)alkyl and (1-4C)alkoxy;
(29) W₁, W₂, W₃ and W₄ are independently selected from CRₕRᵢ, wherein Rₕ and Rᵢ are selected from hydrogen and methyl;
(30) W₁, W₂, W₃ and W₄ are each CH₂;
(31) X₁, X₂, X₃ and X₄ are independently selected from a group of the formula:

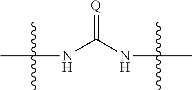

wherein:
[symbol] denotes the point of attachment; and
Q is selected from O, S and NRⱼ, wherein Rⱼ is selected from hydrogen, (1-4C)alkyl and aryl;
(32) X₁, X₂, X₃ and X₄ are independently selected from a group of the formula:

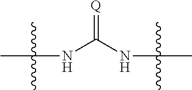

wherein:
[symbol] denotes the point of attachment; and
Q is selected from O and S;
(33) X₁, X₂, X₃ and X₄ are each a group of the formula:

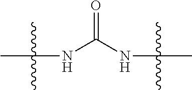 [SMILES: *NC(=O)N*]

wherein:
[symbol] denotes the point of attachment;
(34) Z₁, Z₂, Z₃, Z₄ and Z₅ are independently selected from a hydrophilic substituent group, wherein said hydrophilic substituent comprises one or more hydrophilic functional groups selected from carboxylic acids, carboxylate ions, carboxylate esters, hydroxyl, amines, amides, ethers, ketone and aldehyde groups, ureas, nitro groups, sulphates, sulphonates, phosphates, phosphonates, and combinations thereof;
(35) Z₁, Z₂, Z₃, Z₄ and Z₅ are independently selected from a hydrophilic substituent group, wherein said hydrophilic substituent comprises one or more hydrophilic functional groups selected from carboxylic acids, carboxylate ions, carboxylate esters, hydroxyl, amines, amides, ethers, ketone groups, aldehyde groups and combinations thereof;
(36) Z₁, Z₂, Z₃, Z₄ and Z₅ are independently selected from a hydrophilic substituent group, wherein said hydrophilic substituent comprises one or more hydrophilic functional groups selected from carboxylic acids, carboxylate ions, hydroxyls, amines and combinations thereof;
(37) Z₁, Z₂, Z₃, Z₄ and Z₅ are independently selected from a hydrophilic polymer (e.g. polyethylene glycol), a hydrophilic dendritic group or C(O)OM₁, wherein M₁ is hydrogen or a cation (e.g. Na, Li, NH₄);
(38) Z₁, Z₂, Z₃, Z₄ and Z₅ are independently selected from a hydrophilic polymer (e.g. polyethylene glycol) or a hydrophilic dendritic group;
(39) Z₁, Z₂, Z₃, Z₄ and Z₅ are independently selected from a hydrophilic polymer (e.g. polyethylene glycol) or dendritic group comprising between 1 and 5 generations of building units and a terminal functional group T₁, wherein each building unit is independently selected from a group of Formula A:

L²-L^{2a}-V (Formula A)
wherein:
L² is selected from O, C(O), C(O)O, OC(O), C(O)N(Rr), N(Rr)C(O), N(Rₛ)C(O)N(Rr), N(Rr)C(O)O, OC(O)N(Rᵣ), S(O)₂N(Rᵣ), and N(Rᵣ)SO₂, wherein R_{f} and Rₛ are each independently selected from hydrogen and (1-4C)alkyl;
L²a is a bond or a (1-4C)alkylene;
V is absent or a group of the formula:

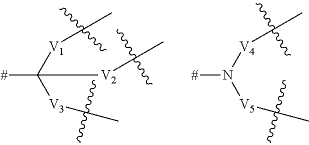

wherein:
V₁, V₂, V₃, V₄ and V₅ are independently selected from a (1-6C)alkylene optionally interrupted by one or more groups selected from O, S and NRₜ, wherein Rₜ is selected from hydrogen and (1-2C)alkyl;
# denotes the point of attachment to one of Rings A, B, C, D or E;
[symbol] denotes the point of attachment to either another group of Formula A or a terminal functional group T₁; and
the terminal functional group T₁ is selected from NH₂, OH, C(O)OMₓ, C(O)ORᵤ and C(O)NHRᵤ, wherein Rᵤ is selected from hydrogen, (1-4C)alkyl, (1-4C)alkoxy, hydroxy(1-4C)alkyl, (2-4C)alkenyl, (2-4C)alkynyl, ethylene glycol and polyethylene glycol, and wherein Mₓ is a cation (e.g. Na, Li, NH₄);
(40) Z₁, Z₂, Z₃, Z₄ and Z₅ are independently selected from a hydrophilic polymer (e.g. polyethylene glycol) or a dendritic group comprising between 1 and 5 generations of building units and a terminal functional group T₁, wherein each building unit is independently selected from a group of Formula A:

-L²-L^{2a}-V— (Formula A)
wherein:
L² is selected from O, C(O), C(O)O, OC(O), C(O)N(Rᵣ) and N(Rᵣ)C(O) wherein R_{f} is selected from hydrogen and (1-4C)alkyl;
L²a is a bond or a (1-4C)alkylene;
V is absent or a group of the formula:

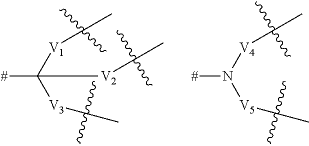

wherein:
V₁, V₂, V₃, V₄ and V₅ are independently selected from a (1-6C)alkylene optionally interrupted by one or more groups selected from O, S and NRₜ, wherein Rₜ is selected from hydrogen and (1-2C)alkyl;
# denotes the point of attachment to one of Rings A, B, C, D or E;
[symbol] denotes the point of attachment to either another group of Formula A or a terminal functional group T₁; and
the terminal functional group T₁ is selected from OH, C(O)OMₓ, C(O)ORᵤ and C(O)NHRᵤ, wherein Rᵤ is selected from hydrogen, (1-4C)alkyl, (1-4C)alkoxy, hydroxy(1-4C)alkyl, (2-4C)alkenyl, (2-4C)alkynyl, ethylene glycol and polyethylene glycol, and wherein Mₓ is a cation (e g. Na, Li, NH₄);
(41) Z₁, Z₂, Z₃, Z₄ and Z₅ are independently selected from a dendritic group comprising between 1 and 4 generations of building units and a terminal functional group T₁, wherein each building unit is independently selected from a group of Formula A:

-L²-L^{2a}-V— (Formula A)
wherein:
L² is selected from O, C(O), C(O)O, OC(O), C(O)N(Rᵣ) and N(Rᵣ)C(O) wherein R_{f} is selected from hydrogen and (1-4C)alkyl;
L²a is a bond or a (1-4C)alkylene;
V is absent or a group of the formula:

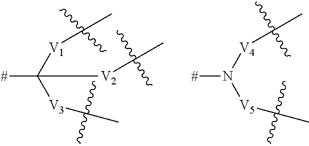

wherein:
V₁, V₂, V₃, V₄ and V₅ are independently selected from a (1-6C)alkylene optionally interrupted by one or more groups selected from O and NRₜ, wherein Rₜ is selected from hydrogen and (1-2C)alkyl;
# denotes the point of attachment to one of Rings A, B, C, D or E;
[symbol] denotes the point of attachment to either another group of Formula A or a terminal functional group T₁; and
the terminal functional group T₁ is selected from OH, C(O)OMₓ, C(O)ORᵤ and C(O)NHRᵤ, wherein Rᵤ is selected from hydrogen, (1-4C)alkyl, (1-4C)alkoxy and hydroxy(1-4C)alkyl, wherein Mₓ is a cation (e.g. Na, Li, NH₄);
(42) Z₁, Z₂, Z₃, Z₄ and Z₅ are independently selected from a hydrophilic polymer or a dendritic group comprising between 1 and 4 generations of building units and a terminal functional group T₁, wherein each building unit is independently selected from a group of Formula A:

-L²-L^{2a}-V— (Formula A)
wherein:
L² is selected from O, C(O), C(O)O and C(O)N(Rᵣ), wherein Rᵣ is selected from hydrogen and (1-4C)alkyl;
L²a is a bond or a (1-4C)alkylene;
V is absent or a group of the formula:

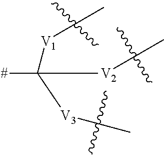

wherein:
V₁, V₂, and V₃ are independently selected from a (1-6C)alkylene optionally interrupted by one or more groups selected from oxygen atoms;
# denotes the point of attachment to one of Rings A, B, C, D or E;
[symbol] denotes the point of attachment to either another group of Formula A or a terminal functional group T₁; and
the terminal functional group T₁ is selected from OH, C(O)OMₓ, C(O)ORᵤ and C(O)NHRᵤ, wherein Rᵤ is selected from hydrogen, (1-4C)alkoxy and hydroxy(1-4C)alkyl, wherein Mₓ is a cation (e g. Na, Li, NH₄);
(43) Z₁, Z₂, Z₃, Z₄ and Z₅ are independently selected from a dendritic group comprising between 1 and 4 generations of building units and a terminal functional group T₁,
wherein each building unit is independently selected from a group of Formula A:

-L²-L^{2a}-V— (Formula A)
wherein:
L² is selected from O, C(O)O and C(O)N(Rᵣ), wherein Rᵣ is selected from hydrogen and (1-4C)alkyl;
L²a is a bond or a (1-4C)alkylene;
V is a group of the formula:

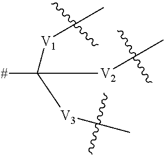

wherein:
V₁, V₂, and V₃ are independently selected from a (1-6C)alkylene optionally interrupted by one or more groups selected from oxygen atoms;
# denotes the point of attachment to one of Rings A, B, C, D or E;
[symbol] denotes the point of attachment to either another group of Formula A or a terminal functional group T₁; and
the terminal functional group T₁ is selected from OH and C(O)OMₓ, wherein Mₓ is a cation (e.g. Na, Li, NH₄);
(44) Z₁, Z₂, Z₃, Z₄ and Z₅ are independently selected from a dendritic group comprising between 1 and 3 generations of building units and a terminal functional group T₁, wherein each building unit is independently selected from a group of Formula A:

L²-L^{2a}-V— (Formula A)
wherein:
L² is C(O)N(Rᵣ), wherein Rᵣ is selected from hydrogen and (1-4C)alkyl;
L²a is a bond or a (1-2C)alkylene;
V is a group of the formula:

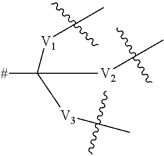

wherein:
V₁, V₂, and V₃ are independently selected from a (1-4C)alkylene optionally interrupted by one or more groups selected from oxygen atoms;
# denotes the point of attachment to one of Rings A, B, C, D or E;
[symbol] denotes the point of attachment to either another group of Formula A or a terminal functional group T₁; and
the terminal functional group T₁ is selected is C(O)OMₓ, wherein Mₓ is a cation (e.g. Na, Li, NH₄);
(45) L is absent or a linker of between 8 and 12 atoms in length (e.g. 10 atoms in length), which optionally bears a hydrophilic substituent group Z₅;
(46) L is absent;
(47) L is a linker of between 8 and 12 atoms in length (e.g. 10 atoms in length), which optionally bears a hydrophilic substituent group Z₅;
(48) L is absent or selected from a group of the formula:

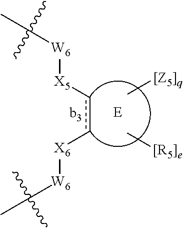

wherein:
[symbol] denotes the point of attachment;
W₅ and W₆ are independently selected from CRₖRᵢ, wherein Rₖ and Rᵢ are selected from hydrogen and (1-2C)alkyl;
X₅ and X₆ are independently selected from a group of the formula:

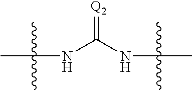

wherein:
[symbol] denotes the point of attachment; and
Q² is selected from O, S and NRₘ, wherein Rₘ, is selected from hydrogen, (1-4C)alkyl, aryl, heteroaryl and sulfonyl;
bond b₃ is a single or double bond;
Ring E is selected from aryl, heteroaryl, heterocyclyl, cycloalkyl and cycloalkenyl;
R₅ is selected from (1-4C)alkyl, halo, (1-4C)haloalkyl, (1-4C)haloalkoxy, (1-4C)alkoxy, (1-4C)alkylamino, amino, cyano, hydroxyl, carboxy, carbamoyl, sulfamoyl and mercapto;
Z₅ is a hydrophilic substituent group as defined herein;
q is an integer from 0 to 2; and
e is an integer from 0 to 2;
(49) L is absent or selected from a group of the formula:

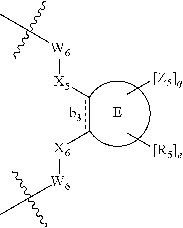

wherein:
[symbol] denotes the point of attachment;
W₅ and W₆ are CH₂;
X₅ and X₆ are independently selected from a group of the formula:

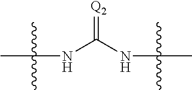

wherein:
[symbol] denotes the point of attachment; and
Q² is selected from O or S;
bond b3 is a single or double bond;
Ring E is selected from aryl and heteroaryl; and
R₅ is selected from (1-4C)alkyl, halo, (1-4C)haloalkyl, (1-4C)haloalkoxy, (1-4C)alkoxy, (1-4C)alkylamino, amino, cyano, hydroxyl, carboxy, carbamoyl, sulfamoyl and mercapto;
Z₅ is a hydrophilic substituent group as defined herein;
q is an integer from 0 to 1; and
e is an integer from 0 to 1;
(50) L is absent or selected from a group of the formula:

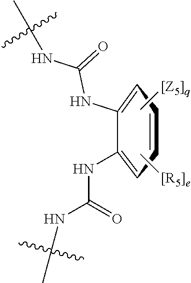

wherein:
R₅ is selected from (1-4C)alkyl, halo, (1-4C)haloalkyl, (1-4C)haloalkoxy, (1-4C)alkoxy, (1-4C)alkylamino, amino, cyano, hydroxyl, carboxy, carbamoyl, sulfamoyl and mercapto;
Z₅ is a hydrophilic substituent group as defined herein;
q is an integer from 0 to 1; and
e is an integer from 0 to 1;
(51) L is absent or selected from a group of the formula:

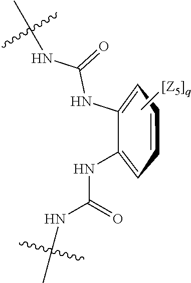

wherein:
R₅ is selected from (1-4C)alkyl, halo, (1-4C)haloalkyl, (1-4C)haloalkoxy, (1-4C)alkoxy, (1-4C)alkylamino, amino, cyano, hydroxyl, carboxy, carbamoyl, sulfamoyl and mercapto;
Z₅ is a hydrophilic substituent group as defined herein; and
q is 1;
(52) c and d are integers independently selected from 0 to 4 (e.g. 0 to 3);
(53) c and d are integers independently selected from 1 to 3;
(54) one of c and d is 3 and the other is an integer selected from 0 to 3;
(55) one of c and d is 3 and the other is an integer selected from 1 to 3;
(56) c and d are both 3;
(57) a and b are integers independently selected from 0 to 1;
(58) a and b are 0;
(59) m, n, o and p are integers independently selected from 0 to 1;
(60) m and n are 1 and o and p are 0.

In paragraphs (39) to (44) above, the term “generation” will be readily understood to refer to the number of layers of building units (e g. groups of Formula A) that make up the dendritic group. The term “generation” is a term of the art commonly used in the field of dendrimer chemistry and will be readily understood by the skilled person. For example, a one generation dendritic group will be understood to have one layer (generation) of building units, e g. −[[building unit]]. A two generation dendritic group has two layers of building units, for example, when the building units have trifunctional branching points, the dendritic group may be: −[[building unit][building unit]₃], a three generation dendritric group has three layers of building units, for example −[[building unit][building unit]₃[building unit]9]. In this regard, the person skilled in the art will readily appreciate that when the dendritic group comprises 1 generation of building units of [symbol] denotes the attachment point to the terminal functional group T₁. Moreover, when the dendritic group comprises 2 generations of building units of Formula A, the person skilled in the art will appreciate that for the first generation of building units of Formula A denotes the attachment point to a second generation of building units of Formula A, and for the second generation of building units of Formula A terminal functional group T₁.

Suitably, a heteroaryl or heterocyclyl group as defined herein is a monocyclic heteroaryl or heterocyclyl group comprising one, two or three heteroatoms selected from N, O or S. Suitably, a heteroaryl is a 5- or 6-membered heteroaryl ring comprising one, two or three heteroatoms selected from N, O or S.

Suitably, a heterocyclyl group is a 4-, 5- or 6-membered heterocyclyl ring comprising one, two or three heteroatoms selected from N, O or S. Most suitably, a heterocyclyl group is a 5-, 6- or 7-membered ring comprising one, two or three heteroatoms selected from N, O or S [e.g. morpholinyl (e g. 4-morpholinyl), pyridinyl, piperazinyl, homopiperazinyl or pyrrolidinonyl].

Suitably an aryl group is phenyl or anthracenyl, most suitably phenyl.

Suitably, bonds b₁ and b₂ are as defined in any one of paragraphs (1) or (2) above.

Suitably, R₁ₐ, R_{1b}, R₂ₐ and R_{2b} are as defined in any one of paragraphs (3) to (5) above.

Most suitably, R₁ₐ, R_{1b}, R₂ₐ and R_{2b} are as defined in paragraph (5) above.

Suitably, Rings A and B are as defined in any one of paragraphs (6) to (10) above. Most suitably, Rings A and B are phenyl.

Suitably, R₁ and R₂ are as defined in any one of paragraphs (11) to (12) above.

Suitably, C and D are as defined in any one of paragraphs (13) to (18) above. Most suitably, C and D are as defined in any one of paragraphs (17) or (18) above.

Suitably, R₃ and R₄ are as defined in any one of paragraphs (19) to (28) above. Most suitably, R₃ and R₄ are as defined in paragraph (28) above.

Suitably, W₁, W₂, W₃ and W₄ are as defined in any one of paragraphs (29) or (30) above.

Suitably, X₁, X₂, X₃ and X₄ are as defined in any one of paragraphs (31) to (33) above. Most suitably, X₁, X₂, X₃ and X₄ are as defined in paragraph (33) above.

Suitably, Z₁, Z₂, Z₃, Z₄ and Z₅ are as defined in any one of paragraphs (34) to (44) above. Most suitably, Z₁, Z₂, Z₃, Z₄ and Z₅ are as defined in paragraph (44) above.

Suitably, L is as defined in any one of paragraphs (45) to (51) above.

Suitably, integers c and d are as defined in any one of paragraphs (52) to (56).

Suitably, integers a and b are as defined in any one of paragraphs (57) to (58) above.

Suitably, integers m, n, o and p are as defined in any one of paragraphs (59) to (60) above.

In a particular group of compounds of the invention, R₁ₐ and R_{1b} together with R₂ₐ and R_{2b} are linked to form Rings A and B respectively, i.e. the compounds have the structural formula Ia (a sub-definition of Formula (I)) shown below, or a salt, hydrate and/or solvate thereof:

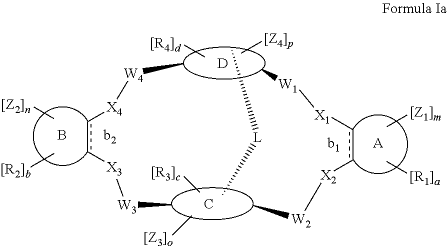

wherein, each of bonds b₁ and b₂, R₁, R₂, R₃, R₄, Z₁, Z₂, Z₃, Z₄, a, b, c, d, m, n, o, p, L, C, D and Rings A and B, are as defined hereinabove.

In an embodiment of the compounds of Formula Ia:
bonds bi and b2 are as defined in any one of paragraphs (1) or (2) above;
Rings A and B are as defined in any one of paragraphs (6) to (10) above;
R₁ and R₂ are as defined in any one of paragraphs (11) to (12) above;
C and D are as defined in any one of paragraphs (13) to (18) above;
R₃ and R₄ are as defined in any one of paragraphs (19) to (28) above;
W₁, W₂, W₃ and W₄ are as defined in any one of paragraphs (29) to (30) above;
X₁, X₂, X₃ and X₄ are as defined in any one of paragraphs (31) to (33) above;
Z₁, Z₂, Z₃, Z₄ and Z₅ are as defined in any one of paragraphs (34) to (44) above;
L is as defined in any one of paragraphs (45) to (51) above;
integers c and d are as defined in any one of paragraphs (52) to (56) above integers a and b are as defined in any one of paragraphs (57) to (58) above; and
integers m, n, 0 and p are as defined in any one of paragraphs (59) to (60) above.

In another embodiment of the compounds of Formula Ia:
bonds b₁ and b₂ are as defined in paragraph (2) above;
Rings A and B are as defined in paragraph (10) above;
R₁ and R₂ are as defined in paragraphs (12) above;
C and D are as defined in any one of paragraphs (17) or (18) above;
R₃ and R₄ are as defined in paragraph (28) above;
W₁, W₂, W₃ and W₄ are as defined in paragraph (30) above;
X₁, X₂, X₃ and X₄ are as defined in paragraph (33) above;
Z₁, Z₂, Z₃, Z₄ and Z₅ are as defined in any one of paragraphs (42) to (44) above;
L is as defined in paragraphs (51) above;
integers c and d are as defined in any one of paragraphs (52) to (56) above;
integers a and b are as defined in any one of paragraphs (57) to (58) above; and
integers m, n, o and p are as defined in any one of paragraphs (59) to (60) above.

In a particular group of compounds of the invention, R₁ₐ and R_{1b} together with R₂ₐ and R_{2b} are linked to form Rings A and B respectively, Q is O and W₁, W₂, W₃ and W₄ are CH₂, i.e. the compounds have the structural formula Ib (a sub-definition of Formula (I)) shown below, or a salt, hydrate and/or solvate thereof:

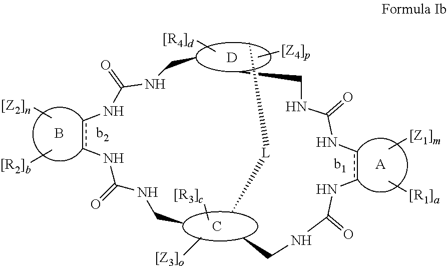

wherein, each of bonds b₁ and b₂, R₁, R₂, R₃, R₄, Z₁, Z₂, Z₃, Z₄, a, b, c, d, m, n, o, p, L, C, D and Rings A and B, are as defined hereinabove.

In an embodiment of the compounds of Formula Ib:
bonds b₁ and b₂ are as defined in any one of paragraphs (1) or (2) above;
Rings A and B are as defined in any one of paragraphs (6) to (10) above;
R₁ and R₂ are as defined in any one of paragraphs (11) to (12) above;
C and D are as defined in any one of paragraphs (13) to (18) above;
R₃ and R₄ are as defined in any one of paragraphs (19) to (28) above;
Z₁, Z₂, Z₃, Z₄ and Z₅ are as defined in any one of paragraphs (34) to (44) above;
L is as defined in any one of paragraphs (45) to (51) above;
integers c and d are as defined in any one of paragraphs (52) to (56) above;
integers a and b are as defined in any one of paragraphs (57) to (58) above; and
integers m, n, o and p are as defined in any one of paragraphs (59) to (60) above.

In another embodiment of the compounds of Formula Ib:
bonds b₁ and b₂ are as defined in paragraph (2) above;
Rings A and B are as defined in paragraph (10) above;
R₁ and R₂ are as defined in paragraphs (12) above;
C and D are as defined in any one of paragraphs (17) or (18) above;
R₃ and R₄ are as defined in paragraph (28) above;
Z₁, Z₂, Z₃, Z₄ and Z₅ are as defined in any one of paragraphs (40) to (44) above;
L is as defined in paragraphs (51) above;
integers c and d are as defined in any one of paragraphs (52) to (56) above;
integers a and b are as defined in any one of paragraphs (57) to (58) above; and
integers m, n, o and p are as defined in any one of paragraphs (59) to (60) above.

In another particular group of compounds of the invention, R₁ₐ and R_{1b} together with R₂ₐ and R_{2b} are linked to form Rings A and B respectively, Q is O, W₁, W₂, W₃ and W₄ are CH₂, and L is as shown below, i.e. the compounds have the structural formula Ic (a sub-definition of Formula (I)) shown below, or a salt, hydrate and/or solvate thereof:

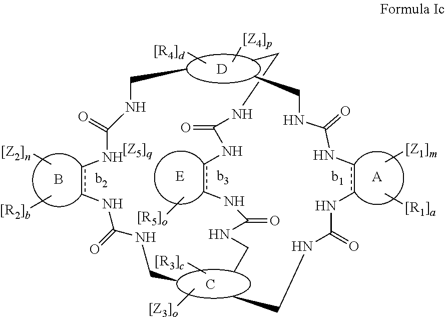

wherein, each of bonds b₁, b₂ and b₃, R₁, R₂, R₃, R₄, R₅, Z₁, Z₂, Z₃, Z₄, Z₅; a, b, c, d, e, m, n, o, p, q, C, D and Rings A, B and E are as defined hereinabove.

In an embodiment of the compounds of Formula Ic:
bonds b₁ and b₂ are as defined in any one of paragraphs (1) or (2) above;
Rings A and B are as defined in any one of paragraphs (6) to (10) above;
R₁ and R₂ are as defined in any one of paragraphs (11) to (12) above;
C and D are as defined in any one of paragraphs (13) to (18) above;
R₃ and R₄ are as defined in any one of paragraphs (19) to (28) above;
Z₁, Z₂, Z₃, Z₄ and Z₅ are as defined in any one of paragraphs (34) to (44) above;
bond b₃, Ring E, Rₛ and integers e and q are as defined in any one of paragraphs (48) to (49) above;
integers c and d are as defined in any one of paragraphs (52) to (56) above;
integers a and b are as defined in any one of paragraphs (57) to (58) above; and
integers m, n, o and p are as defined in any one of paragraphs (59) to (60) above.

In another embodiment of the compounds of Formula Ic:
bonds b₁ and b₂ are as defined in paragraph (2) above;
Rings A and B are as defined in paragraph (10) above:
R₁ and R₂ are as defined in paragraph (12) above;
C and D are as defined in any one of paragraphs (17) to (18) above;
R₃ and R₄ are as defined in paragraph (28) above;
Z₁, Z₂, Z₃, Z₄ and Z₅ are as defined in any one of paragraphs (40) to (44) above; bond b₃, Ring E, Rₛ and integers e and q are as defined in paragraph (49) above; integers c and d are as defined in any one of paragraphs (52) to (56) above; integers a and b are as defined in any one of paragraphs (57) to (58) above; and integers m, n, o and p are as defined in any one of paragraphs (59) to (60) above.

In yet another particular group of compounds of the invention, Q is O, W₁, W₂, W₃ and W₄ are CH₂, and L is as shown below, Rings A, B and E are phenyl, integers m and n are 1 and integers a, b and e are 0, i.e. the compounds have the structural formula Id (a sub-definition of Formula (I)) shown below, or a salt, hydrate and/or solvate thereof:

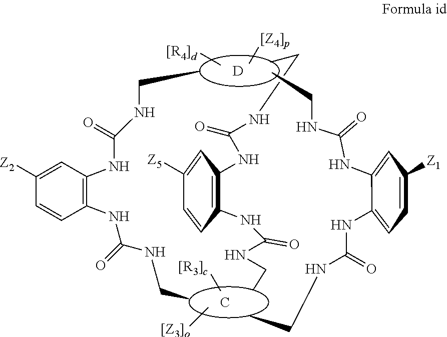

wherein, each of R₃, R₄, Z₁, Z₂, Z₃, Z₄, Z₅, c, d, o, p and Rings C and D are as defined hereinabove.

In an embodiment of the compounds of Formula Id:
Rings C and D are as defined in any one of paragraphs (13) to (18) above;
R₃ and R₄ are as defined in any one of paragraphs (19) to (28) above;
Z₁, Z₂, Z₃, Z₄ and Z₅ are as defined in any one of paragraphs (34) to (44) above;
integers c and d are as defined in any one of paragraphs (42) to (56) above; and
integers o and p are as defined in any one of paragraphs (59) to (60) above.

In another particular group of compounds of the invention, Q is O, W₁, W₂, W₃ and W₄ are CH₂, and L is as shown below, Rings A, B and E are phenyl, integers m and n are 1 and integers a, b and e are 0, i.e. the compounds have the structural formula Ie (a sub-definition of Formula (I)) shown below, or a salt, hydrate and/or solvate thereof:

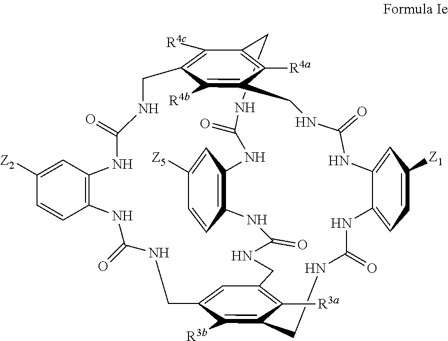

wherein, each of Z₁, Z₂, Z₃, Z₄ and Z₅ are as defined hereinabove and R₃ₐ, R_{3b}, R_{3c}, R₄ₐ, R_{4b} and R_{4c} are independently selected from hydrogen, halo, (1-4C)alkyl, (1-4C)alkoxy, amino, nitro, (1-4C)alkylamino, (1-4C)dialkylamino, (1-4C)haloalkyl, (1-4C)haloalkoxy, cyano, (2-4C)alkenyl, (2-4C)alkynyl and a group of the formula:

-L^{1a}-Y^{1a}-Q^{1a}
wherein:
L¹a is absent or (1-2C)alkylene optionally substituted by one or more substituents selected from (1-2C)alkyl and oxo;
Y^{1a} is absent or O, S, SO, SO₂, N(Rₙ), C(O), C(O)O, OC(O), C(O)N(Rₙ) and N(Rₙ)C(O), wherein Rₙ is selected from hydrogen and (1-4C)alkyl; and
Q^{1a} is hydrogen, (1-8C)alkyl, (2-6C)alkenyl, (2-6C)alkynyl, aryl, (3-10C)cycloalkyl, (3-10C)cycloalkenyl, heteroaryl and heterocyclyl; wherein Q^{1a} is optionally further substituted by one or more substituent groups independently selected from (1-4C)alkyl, halo, (1-4C)haloalkyl, (1-4C)haloalkoxy, amino, (1-4C)aminoalkyl, cyano, hydroxy, carboxy, carbamoyl, sulfamoyl, mercapto, ureido, oxy, NRₒRₚ, ORₒ, C(O)Rₒ, C(O)ORₒ, OC(O)Rₒ, C(O)N(Rₚ)Rₒ, N(Rₚ)C(O)Rₒ, S(O)_{y1}Rₒ(where y1 is 0, 1 or 2), SO₂N(Rₚ)Rₒ, N(Rₚ)SO₂Rₒ, Si(R_{q})(Rₚ)Rₒ and (CH₂)_{z1}NRₒRₚ (where z1 is 1, 2 or 3); wherein Rₒ, Rₚ and R_{q} are each independently selected from hydrogen and (1-6C)alkyl.

In an embodiment of the compounds of Formula Ie:
Z₁, Z₂, Z₃, Z₄ and Z₅ are as defined in any one of paragraphs (34) to (42) above; and
R₃ₐ, R_{3b}, R_{3c}, R₄ₐ, R_{4b} and R_{4c} are independently selected from hydrogen, halo, (1-4C)alkyl, (1-4C)alkoxy, amino, nitro, (1-4C)alkylamino, (1-4C)dialkylamino, (1-4C)haloalkyl, (1-4C)haloalkoxy, cyano, (2-4C)alkenyl, (2-4C)alkynyl and a group of the formula:

-L^{1a}-Y^{1a}-Q^{1a}
wherein:
L¹a is absent or (1-2C)alkylene;
Y^{1a} is absent or O, S, SO, SO₂, N(Rₜ), C(O), C(O)O, OC(O), C(O)N(Rₙ) and N(Rₙ)C(O), wherein Rₙ is selected from hydrogen and (1-4C)alkyl; and
Q^{1a} is hydrogen, (1-8C)alkyl, (2-6C)alkenyl, (2-6C)alkynyl, aryl, (3-10C)cycloalkyl, (3-10C)cycloalkenyl, heteroaryl or heterocyclyl; wherein Q^{1a} is optionally further substituted by one or more substituent groups independently selected from (1-4C)alkyl, halo, (1-4C)haloalkyl, (1-4C)haloalkoxy, amino, (1-4C)aminoalkyl, cyano, hydroxy, carboxy, carbamoyl, sulfamoyl and mercapto.

In another embodiment of the compounds of Formula Ie:
Z₁, Z₂, Z₃, Z₄ and Z₅ are as defined in paragraph (42) above; and
R₃ₐ, R_{3b}, R_{3c}, R₄ₐ, R_{4b} and R_{4c} are independently selected from hydrogen, halo, (1-4C)alkyl, (1-4C)alkoxy, amino, nitro, (1-4C)alkylamino, (1-4C)dialkylamino, (1-4C)haloalkyl, (1-4C)haloalkoxy, cyano, (2-4C)alkenyl and (2-4C)alkynyl.

In another embodiment of the compounds of Formula Ie:
Z₁, Z₂, Z₃, Z₄ and Z₅ are as defined in paragraph (42) above; and
R₃ₐ, R_{3b}, R_{3c}, R₄ₐ, R_{4b} and R_{4c} are independently selected from hydrogen, halo, (1-4C)alkyl, (1-4C)alkoxy, amino, nitro, (1-4C)alkylamino, (1-4C)dialkylamino, (1-4C)haloalkyl, (1-4C)haloalkoxy, cyano, (2-4C)alkenyl and (2-4C)alkynyl, with the proviso that R₃ₐ, R_{3b} and R₃, cannot all be hydrogen.

In another embodiment of the compounds of Formula Ie:
Z₁, Z₂, Z₃, Z₄ and Z₅ are as defined in paragraph (42) above; and
R₃ₐ, R_{3b}, R_{3c}, R₄ₐ, R_{4b} and R_{4c} are independently selected from halo, (1-4C)alkyl, (1-4C)alkoxy, amino, nitro, (1-4C)alkylamino, (1-4C)dialkylamino, (1-4C)haloalkyl, (1-4C)haloalkoxy, cyano, (2-4C)alkenyl and (2-4C)alkynyl.

In another embodiment of the compounds of Formula Ie:
Z₁, Z₂, Z₃, Z₄ and Z₅ are as defined in paragraph (44) above; and
R₃ₐ, R_{3b}, R_{3c}, R₄ₐ, R_{4b} and R_{4c} are independently selected from hydrogen and (1-4C)alkyl.

In another embodiment of the compounds of Formula Ie:
Z₁, Z₂, Z₃, Z₄ and Z₅ are as defined in paragraph (44) above; and
R₃ₐ, R_{3b}, R_{3c}, R₄ₐ, R_{4b} and R_{4c} are independently selected from hydrogen, (1-4C)alkoxy and (1-4C)alkyl.

In another embodiment of the compounds of Formula Ie:
Z₁, Z₂, Z₃, Z₄ and Z₅ are as defined in paragraph (44) above; and
R₃ₐ, R_{3b}, R_{3c}, R₄ₐ, R_{4b} and R_{4c} are independently selected from (1-4C)alkoxy and (1-4C)alkyl.

In another embodiment of the compounds of Formula Ie:
Z₁, Z₂, Z₃, Z₄ and Z₅ are as defined in paragraph (44) above; and
R₃ₐ, R_{3b}, R_{3c}, R₄ₐ, R_{4b} and R_{4c} are independently a (1-4C)alkyl (e.g. ethyl).

In yet a further group of compounds of the invention, R₁ₐ and R_{1b} together with R₂ₐ and R_{2b} are linked to form Rings A and B respectively, Q is O, L is absent and W₁, W₂, W₃ and W₄ are CH₂, i.e. the compounds have the structural formula If (a sub-definition of Formula (I)) shown below, or a salt, hydrate and/or solvate thereof:

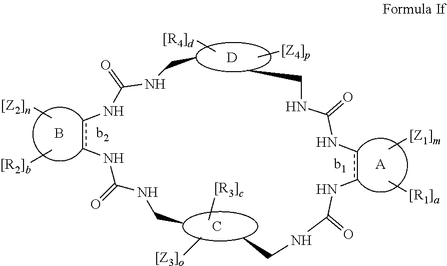

wherein, each of bonds b₁ and b₂, R₁, R₂, R₃, R₄, Z₁, Z₂, Z₃, Z₄, a, b, c, d, m, n, o, p, C, D and Rings A and B are as defined hereinabove.

In an embodiment of the compounds of Formula If:
bonds b₁ and b₂ are as defined in any one of paragraphs (1) or (2) above;
Rings A and B are as defined in any one of paragraphs (6) to (10) above;
R₁ and R₂ are as defined in any one of paragraphs (11) to (12) above;
C and D are as defined in any one of paragraphs (13) to (18) above;
R₃ and R₄ are as defined in any one of paragraphs (19) to (28) above;
Z₁, Z₂, Z₃ and Z₄ are as defined in any one of paragraphs (34) to (44) above;
integers c and d are as defined in any one of paragraphs (52) to (56) above;
integers a and b are as defined in any one of paragraphs (57) to (58) above; and
integers m, n, o and p are as defined in any one of paragraphs (59) to (60) above.

In another embodiment of the compounds of Formula If:
bonds b₁ and b₂ are as defined in paragraph (2) above;
Rings A and B are as defined in paragraph (10) above:
R₁ and R₂ are as defined in paragraph (12) above;
C and D are as defined in any one of paragraphs (17) to (18) above;
R₃ and R₄ are as defined in paragraph (28) above;
Z₁, Z₂, Z₃ and Z₄ are as defined in any one of paragraphs (38) to (44) above;
integers c and d are as defined in any one of paragraphs (52) to (56) above;
integers a and b are as defined in any one of paragraphs (57) to (58) above; and
integers m, n, o and p are as defined in any one of paragraphs (52) to (53) above.

In another particular group of compounds of the invention, Q is O, L is absent, W₁, W₂, W₃ and W₄ are CH₂, Rings A and B are phenyl and Rings C and D are anthracenyl, i.e. the compounds have the structural formula Ig (a sub-definition of Formula (I)) shown below, or a salt, hydrate and/or solvate thereof:

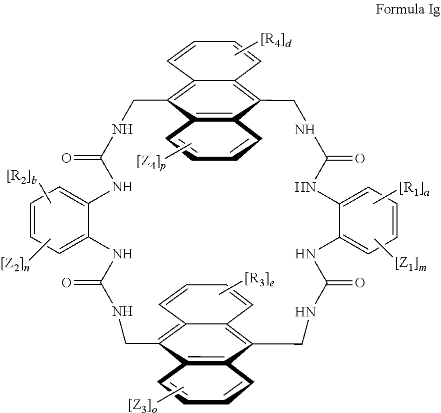

wherein each of R₁, R₂, R₃, R₄, Z₁, Z₂, Z₃, Z₄, a, b, c, d, m, n, o and p are as defined hereinabove.

In an embodiment of the compounds of Formula Ig:
R₁ and R₂ are as defined in any one of paragraphs (11) to (12) above;
R₃ and R₄ are as defined in any one of paragraphs (19) to (28) above;
Z₁, Z₂, Z₃ and Z₄ are as defined in any one of paragraphs (34) to (44) above; integers c and d are as defined in paragraphs (52) above;
integers a and b are as defined in any one of paragraphs (57) to (58) above; and
integers m, n, 0 and p are as defined in any one of paragraphs (59) to (60) above.

Particular compounds of the present invention include any of the compounds exemplified in the present application, or a salt, solvate or hydrate thereof, and, in particular, any of the following:

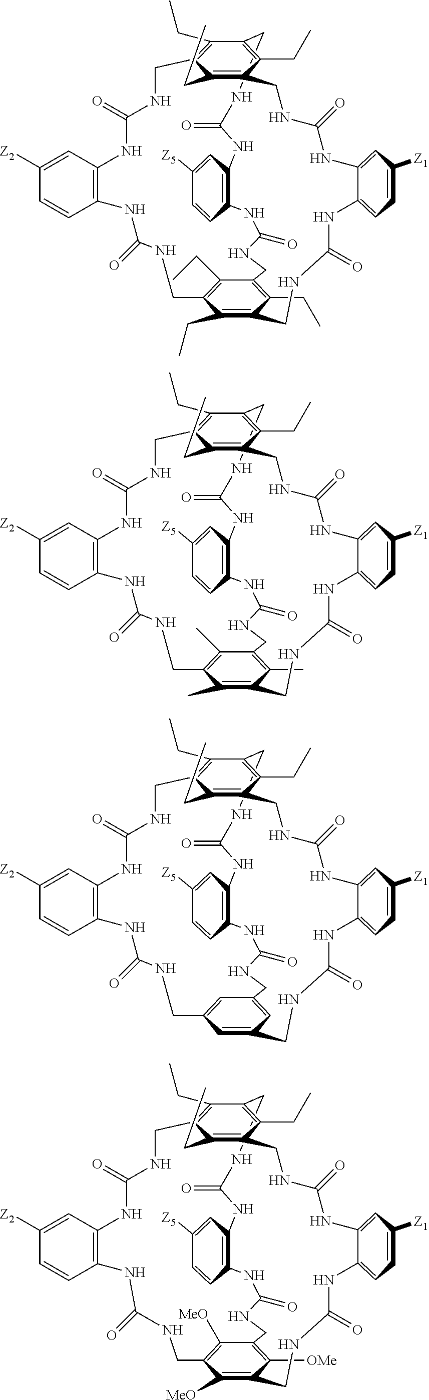

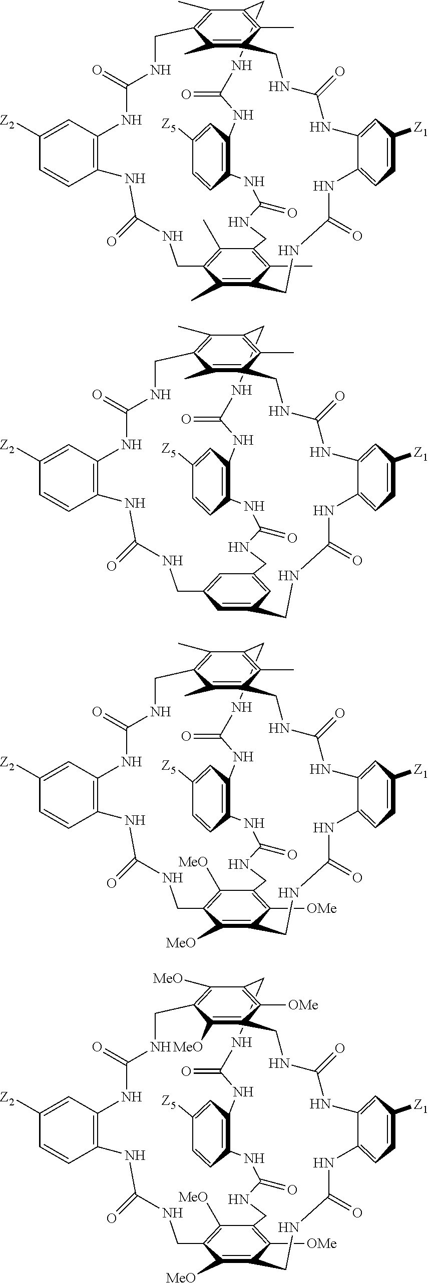

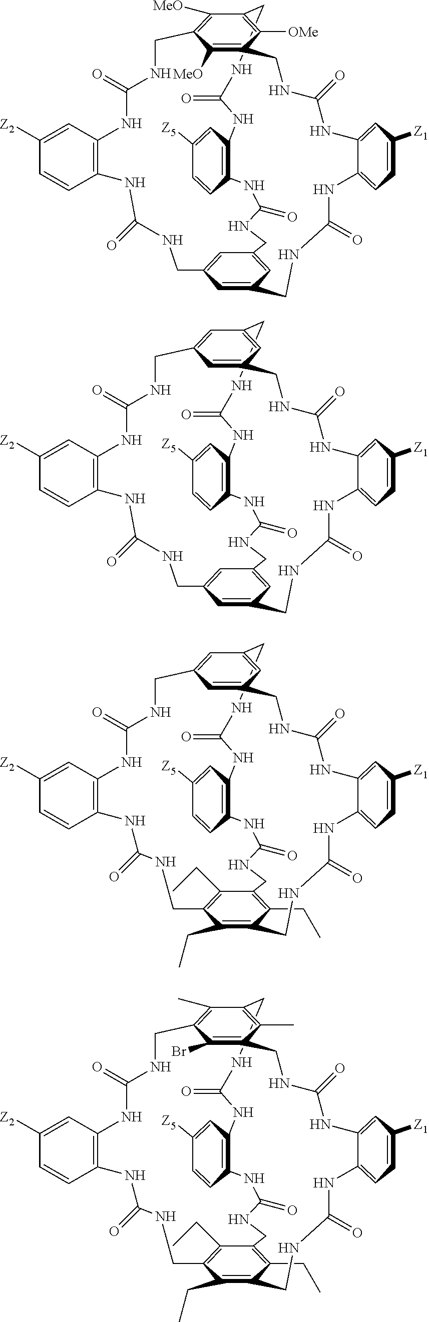

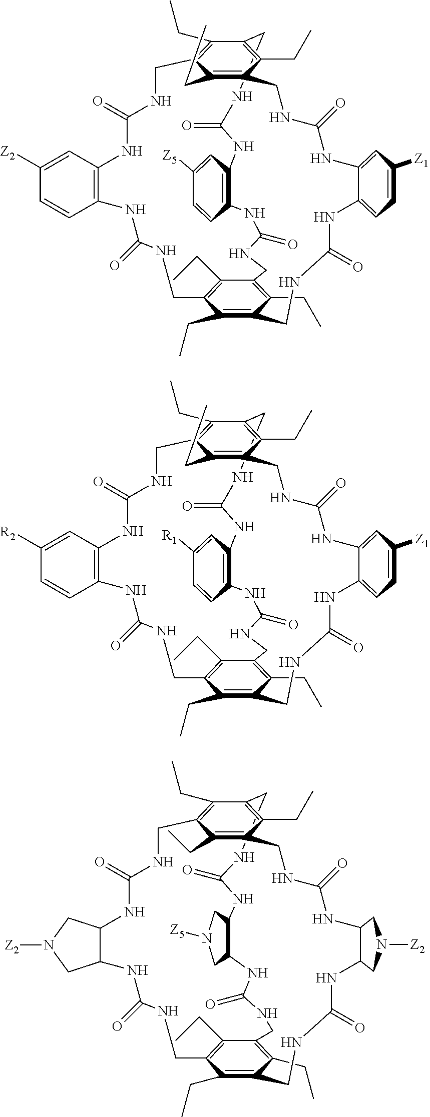

wherein:
each of Z₁, Z₂ and Z₅ are independently selected from one of the following group:

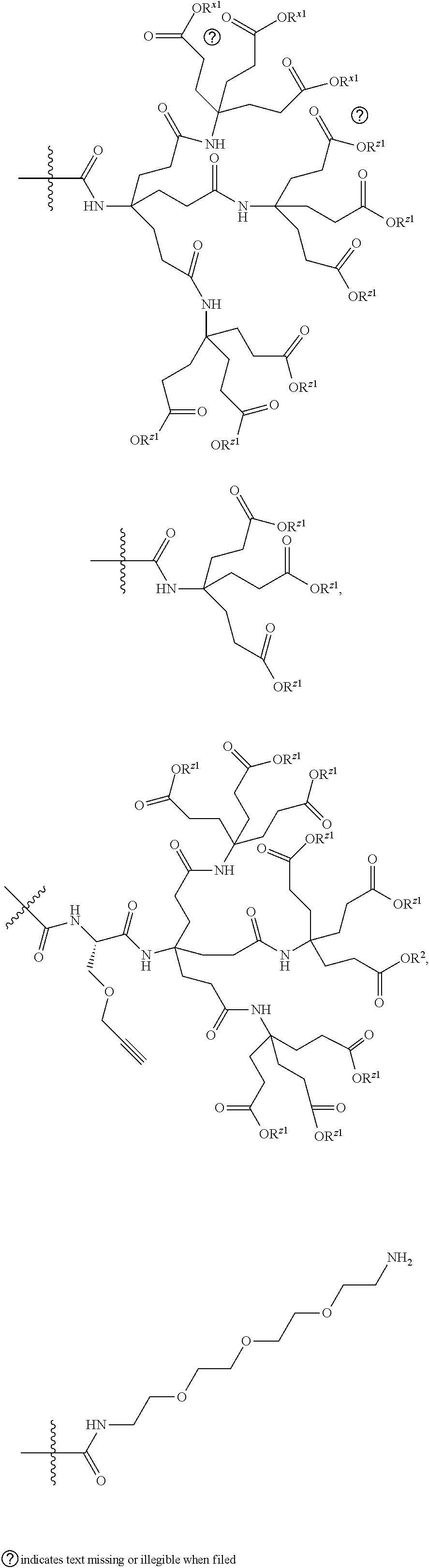

wherein each R^{z1} is independently selected from hydrogen or Na (i.e. the carboxy group is either a carboxylic acid or a sodium salt thereof);
R₁ and R₂ are groups of the formula:

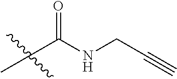 [SMILES: *C(=O)NCC#C]

where [symbol] denotes the point of attachment; and
optionally any of Z₁, Z₂, Z₅, R₁ and R₂ are replaced with one of the macrocyle tether group is as defined herein.

Particular embodiments of the saccharide-binding compound include:

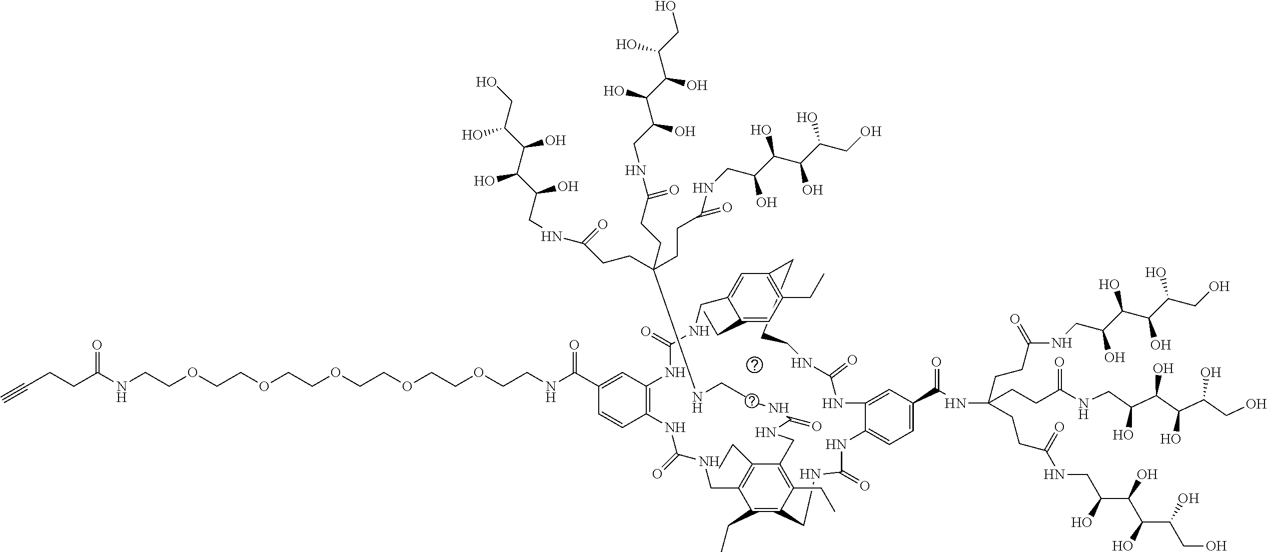

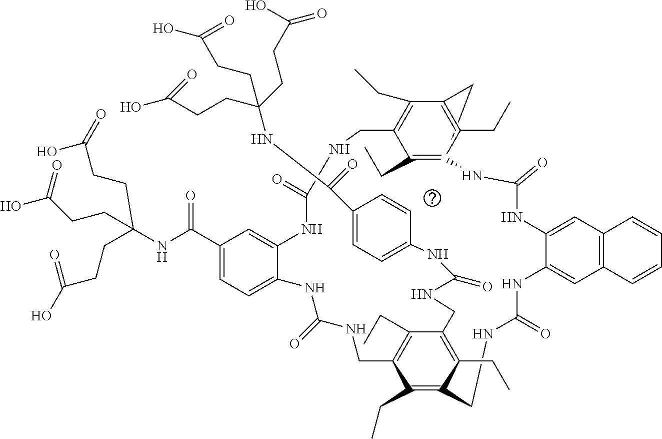

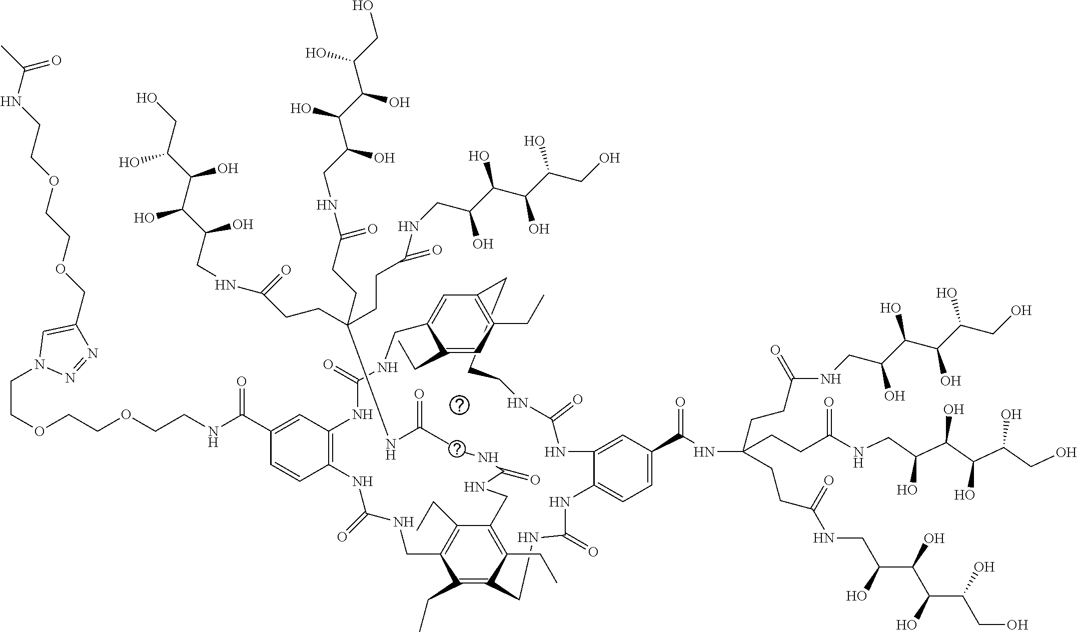

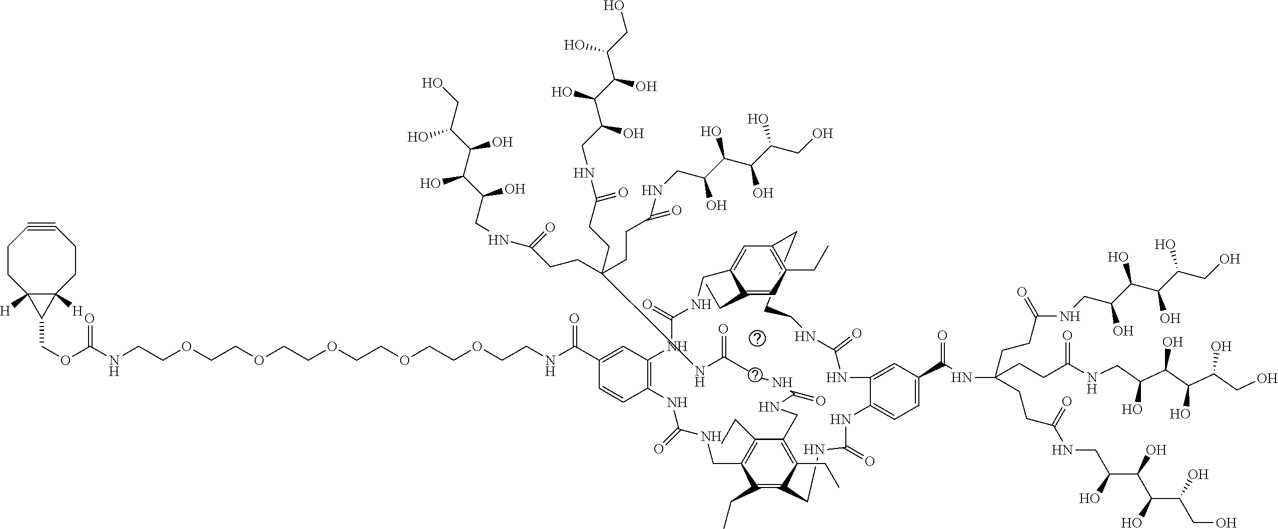

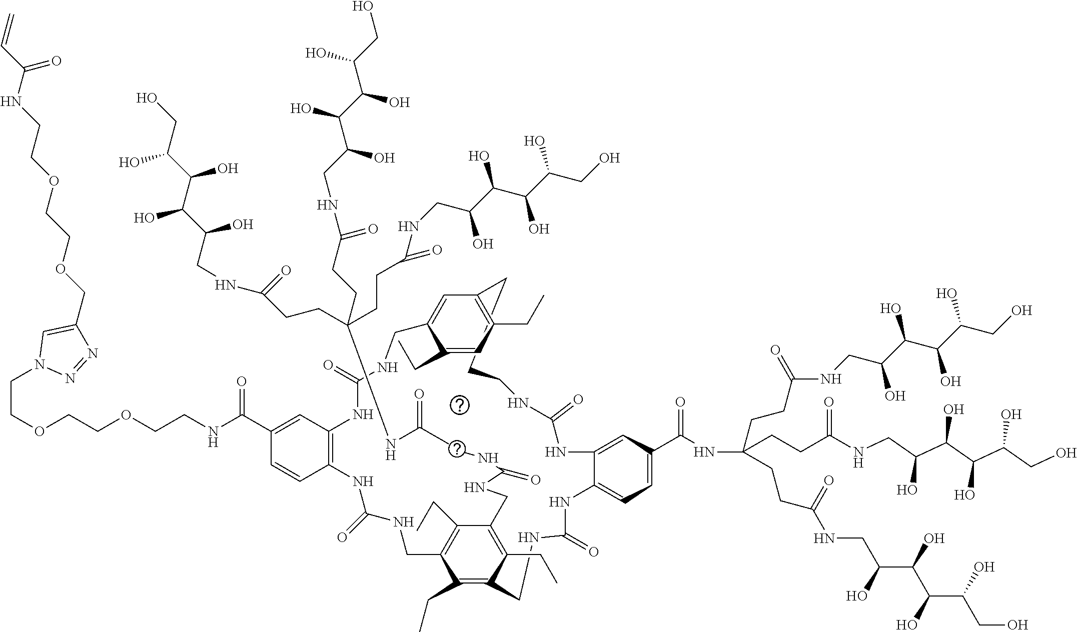

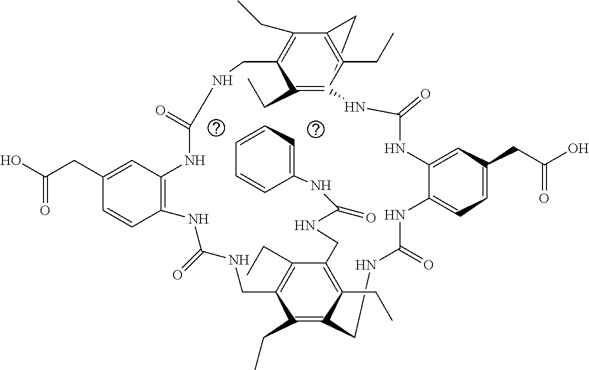

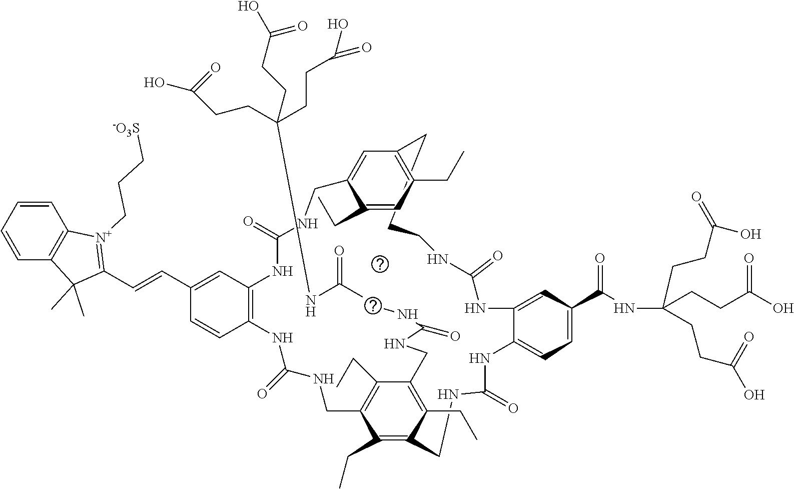

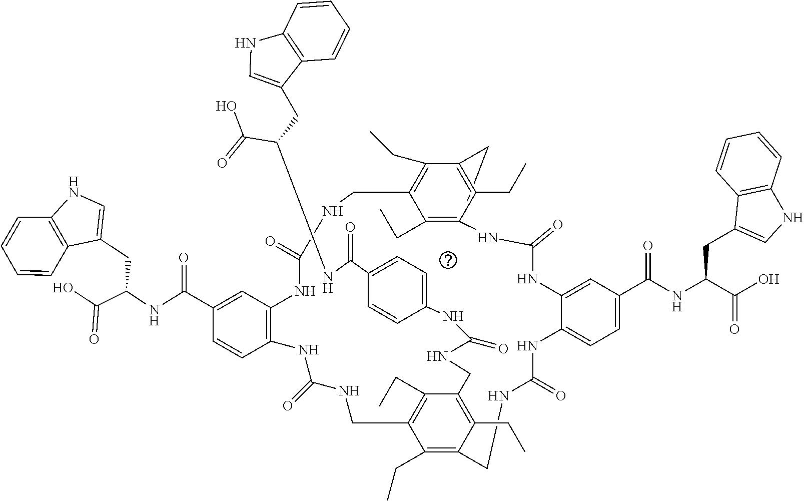 [SMILES: CCc1c(CNC(=O)Nc2ccc(C(=O)N[C@@H](Cc3c[nH]c4ccccc34)C(=O)O)cc2)c(CC)c2c(CC)c1CNC(=O)Nc1ccc(C(=O)N[C@@H](Cc3c[nH]c4ccccc34)C(=O)O)cc1NC(=O)NCc1c(CC)c(CC)c(CC)c(c1CC)NC(=O)Nc1cc(C(=O)N[C@@H](Cc3c[nH]c4ccccc34)C(=O)O)ccc1NC(=O)NC2]

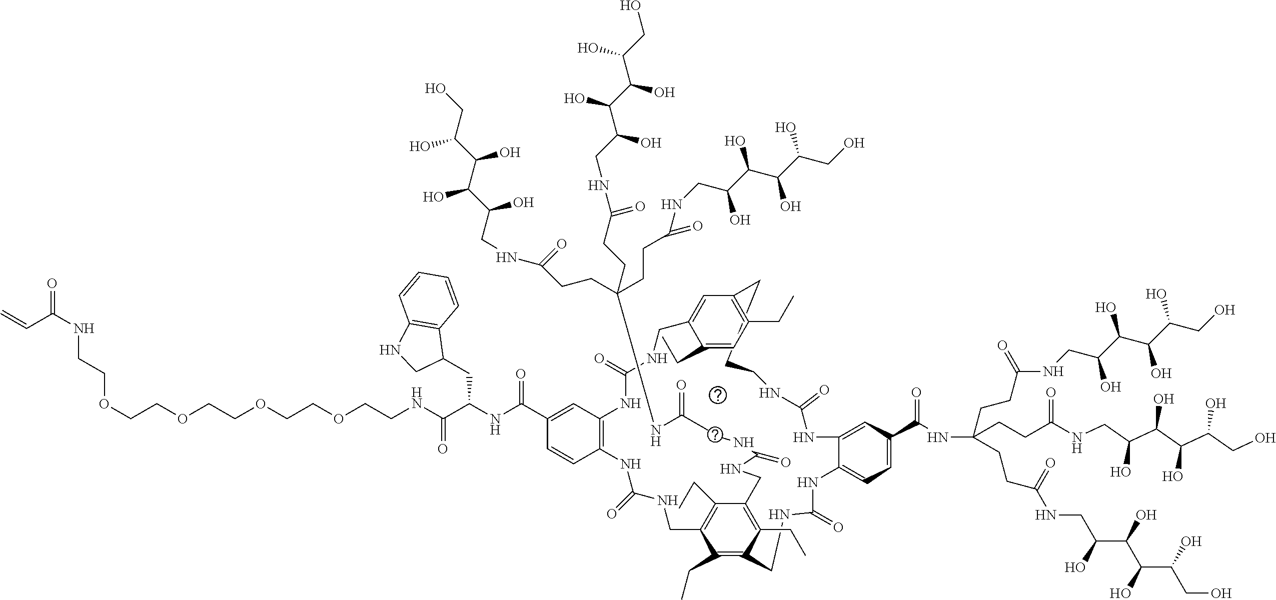

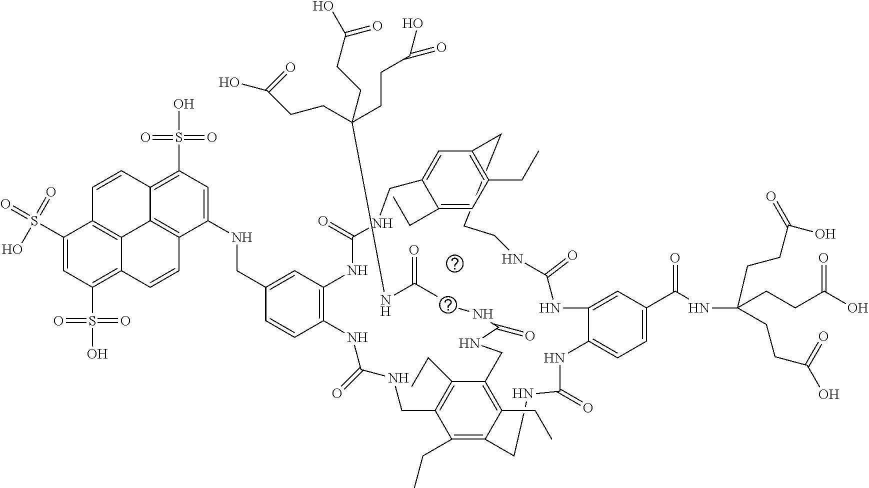

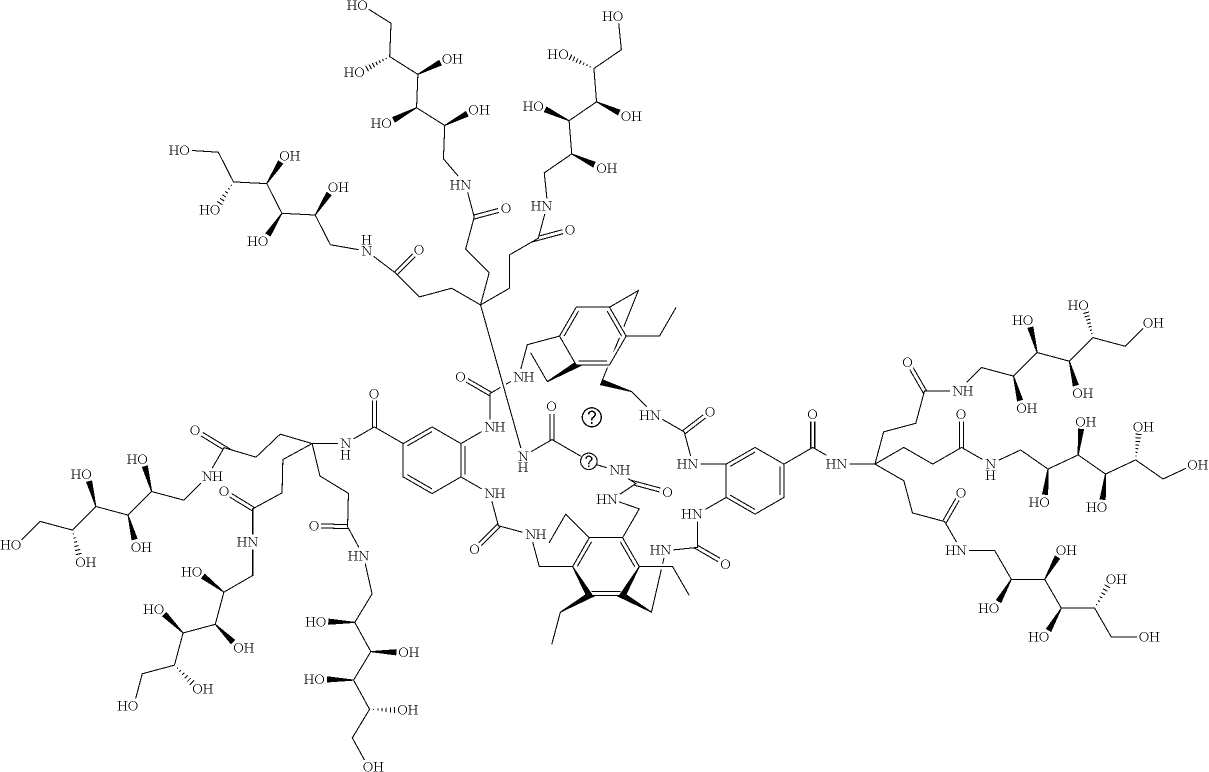

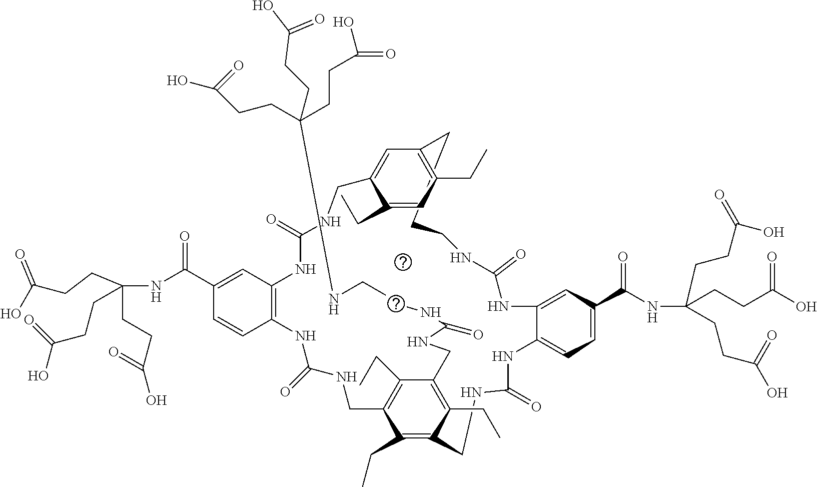

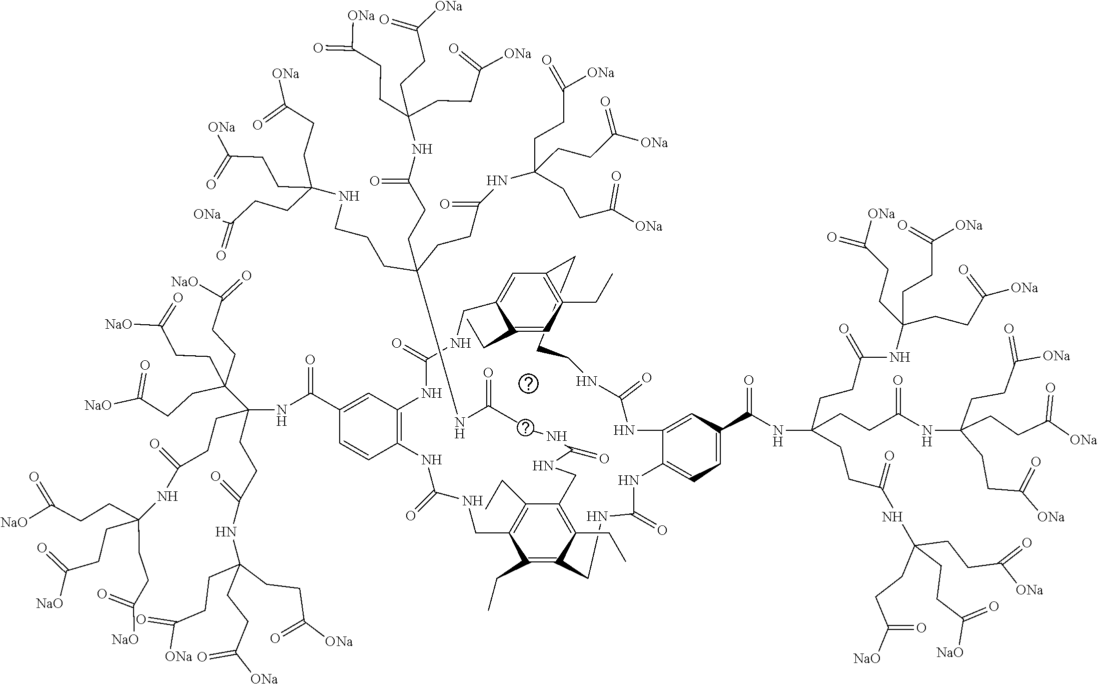

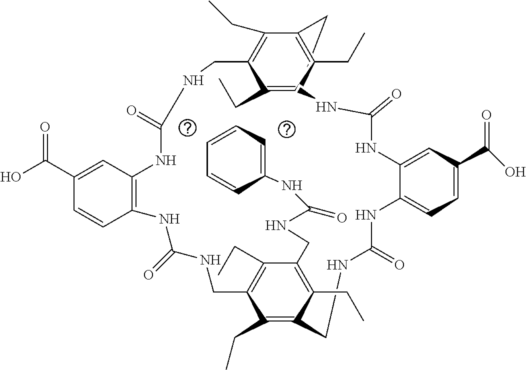

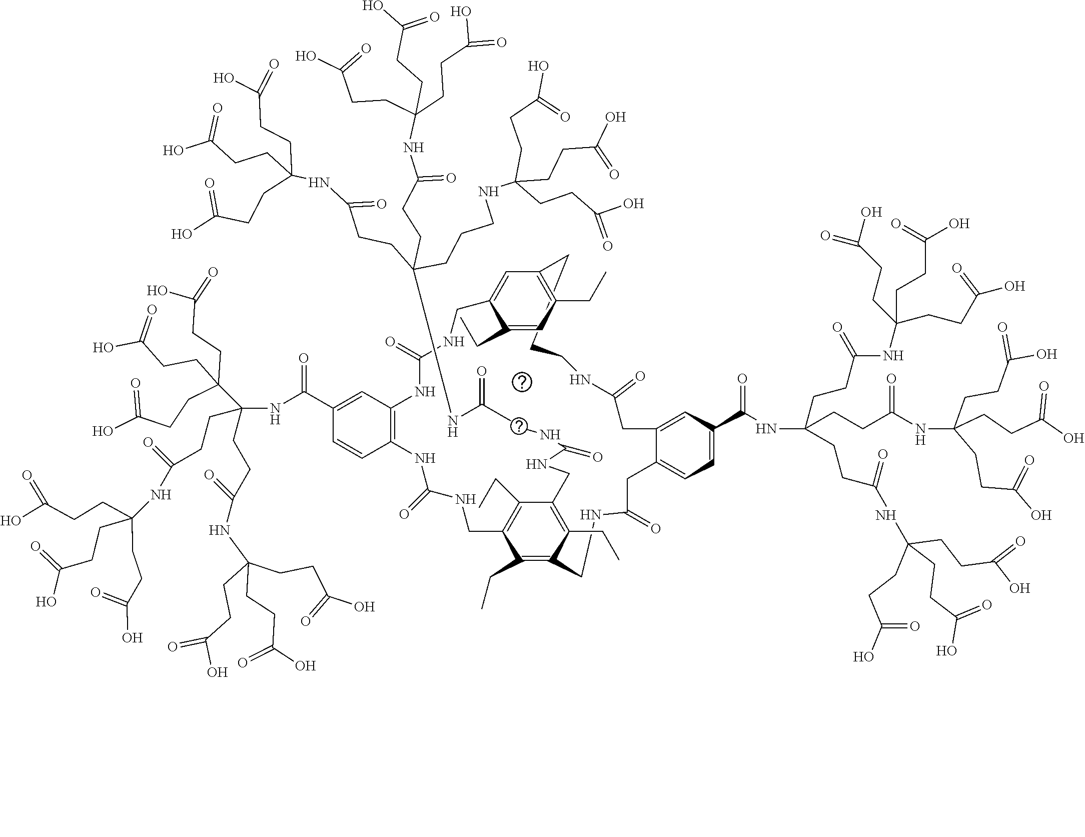

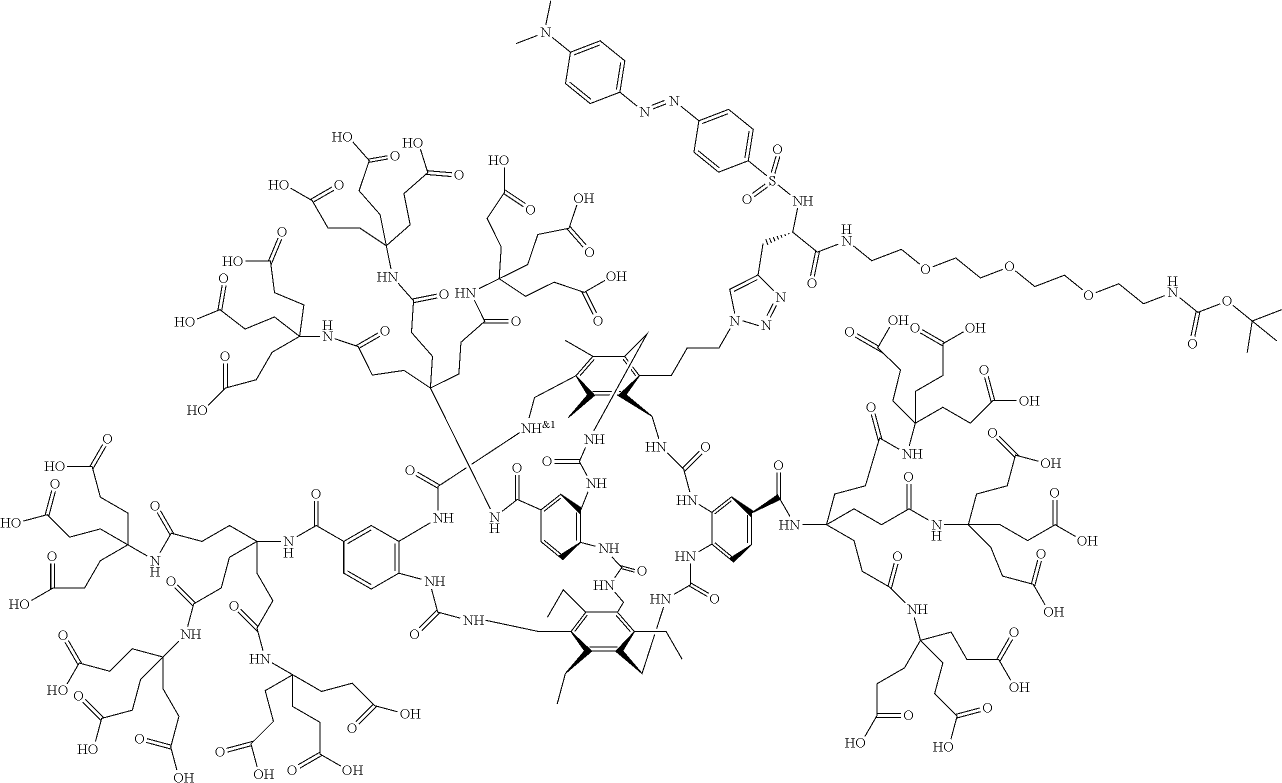

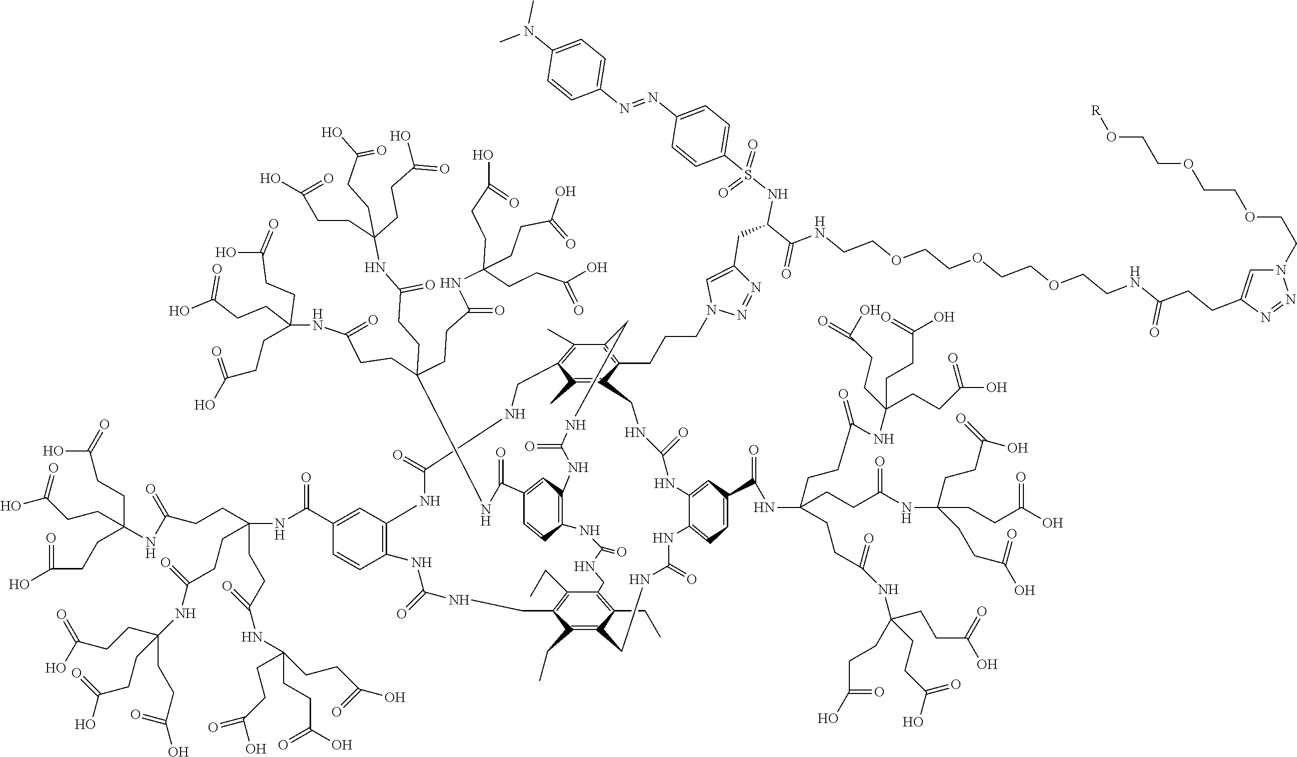

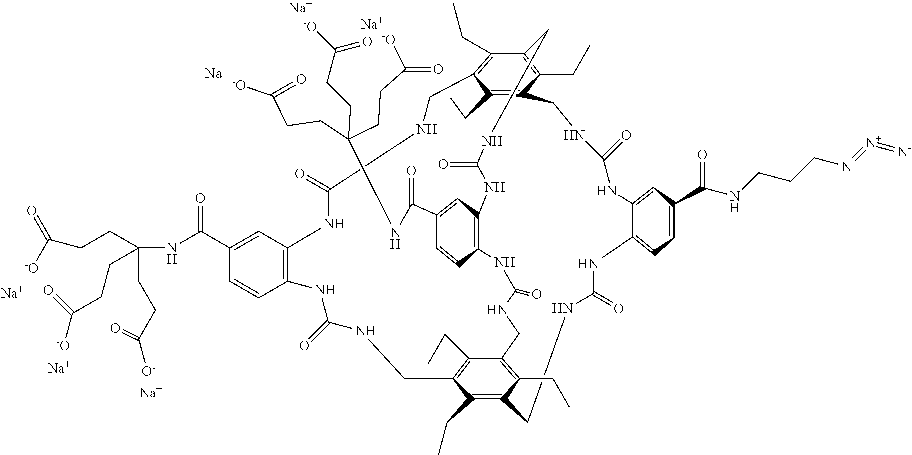

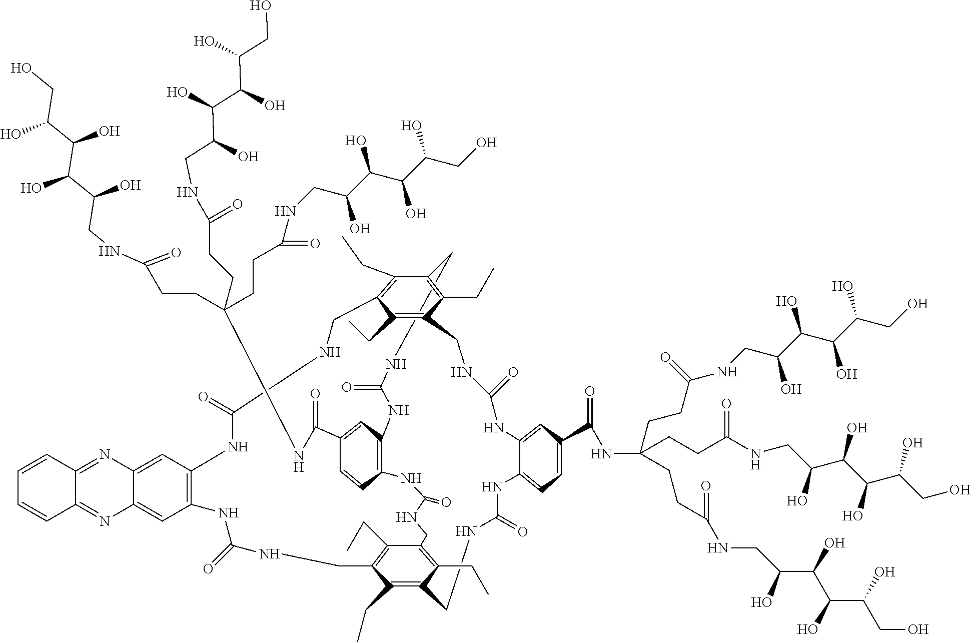

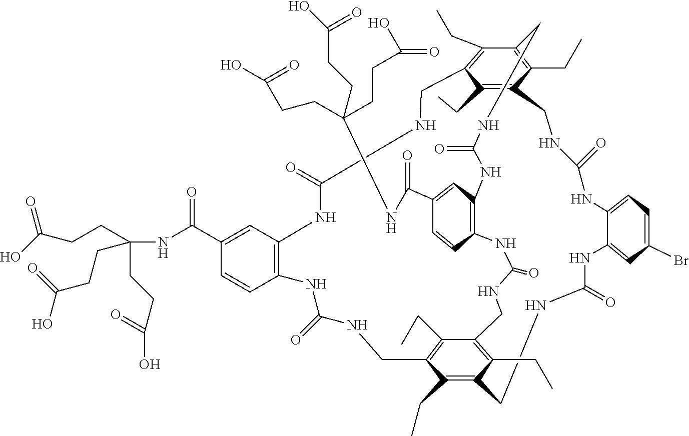

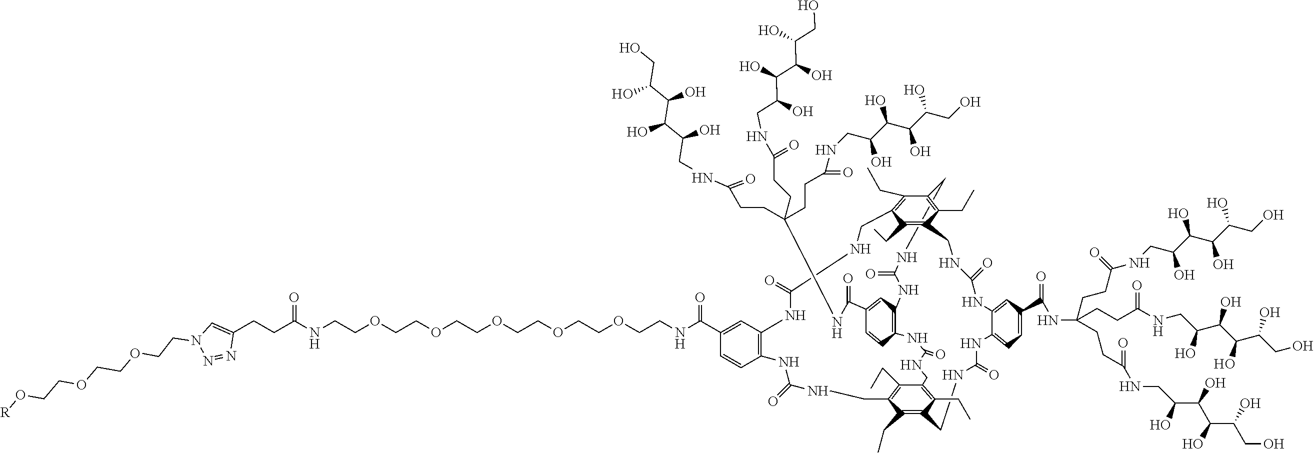

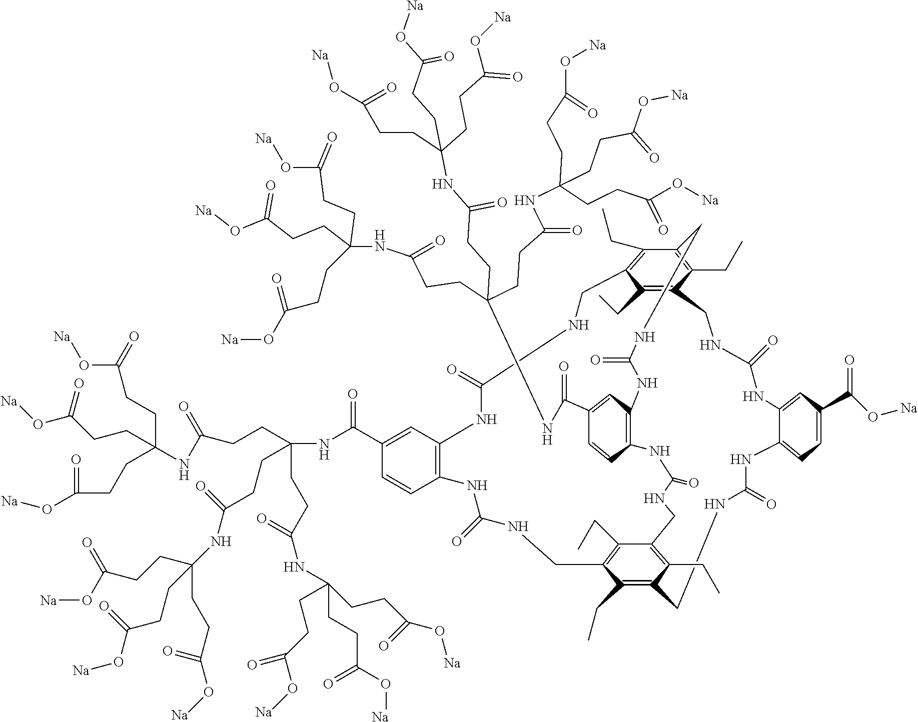

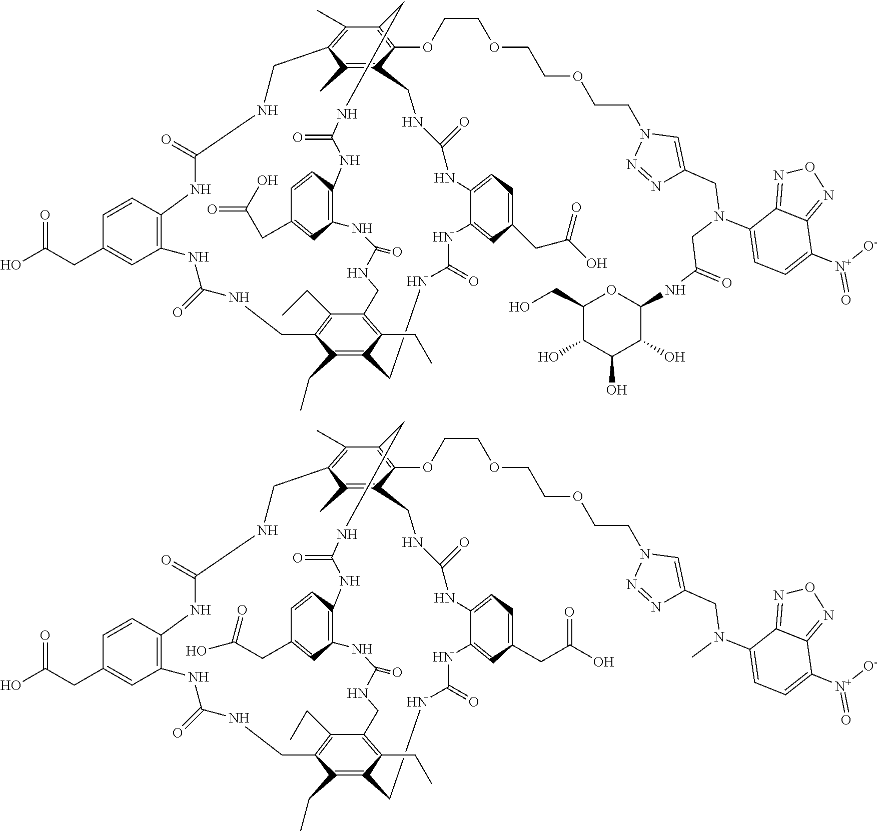

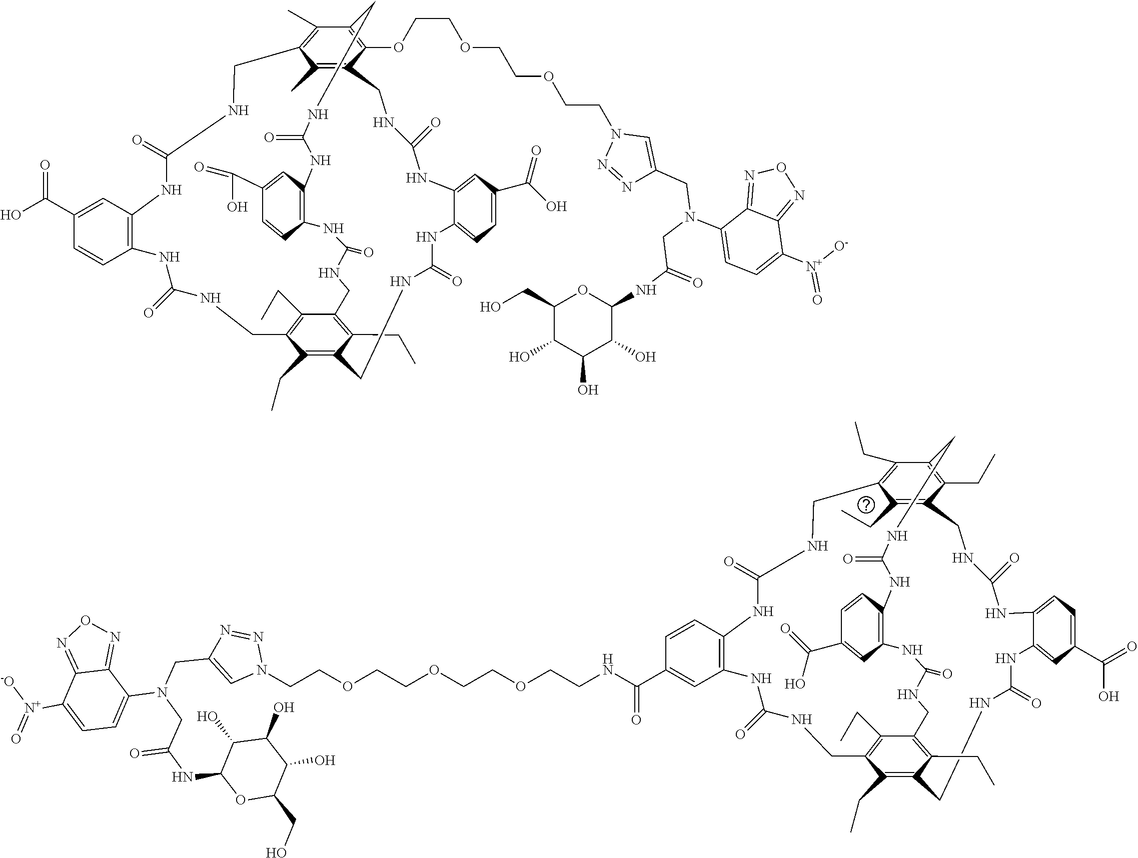

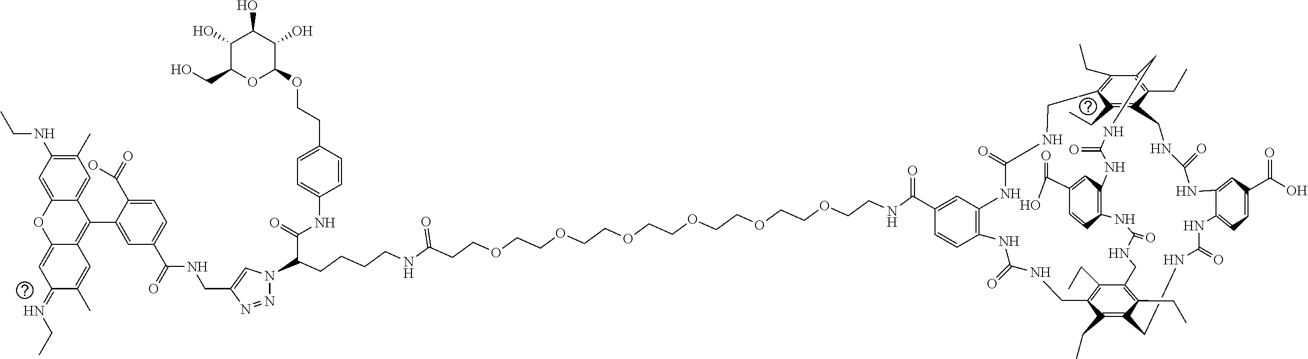

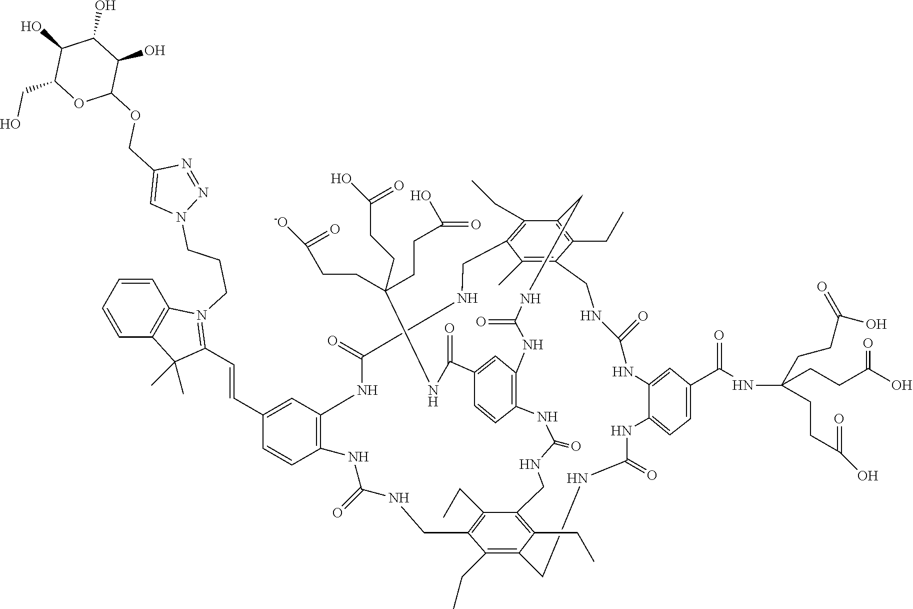

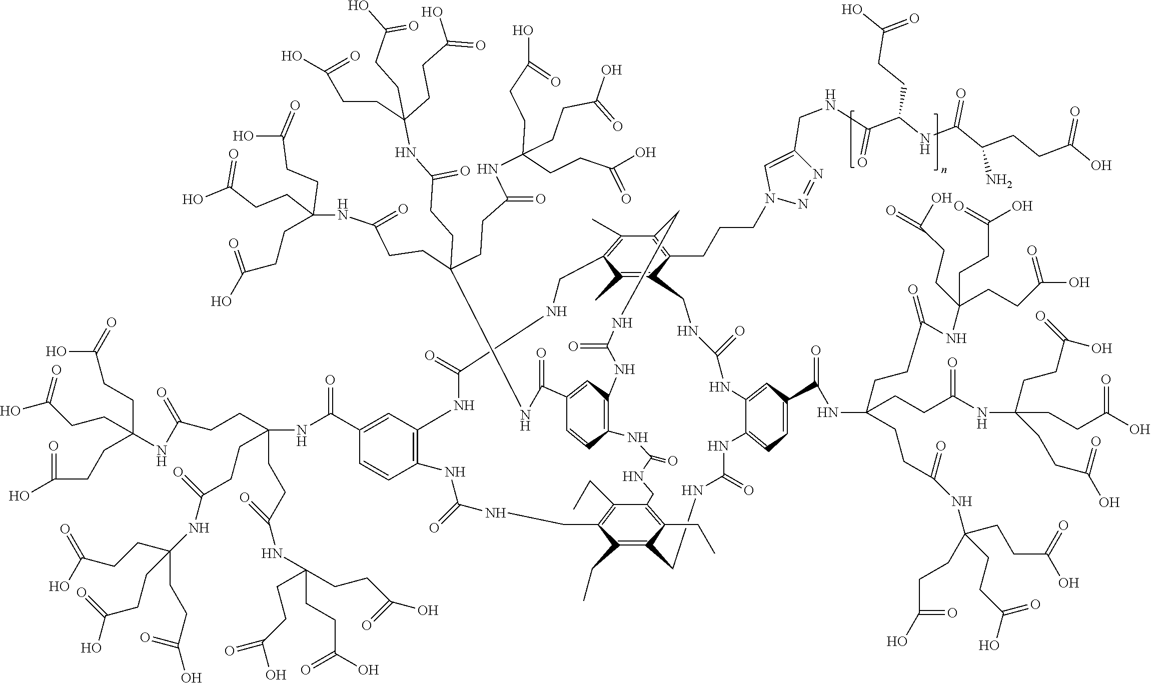

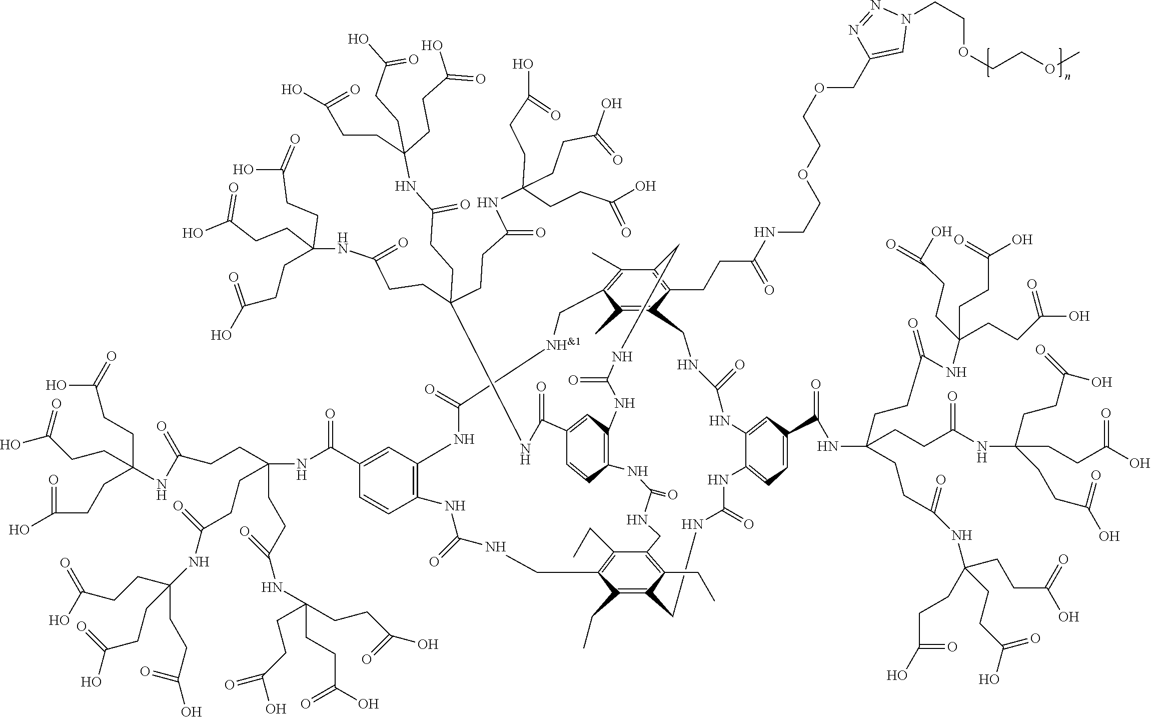

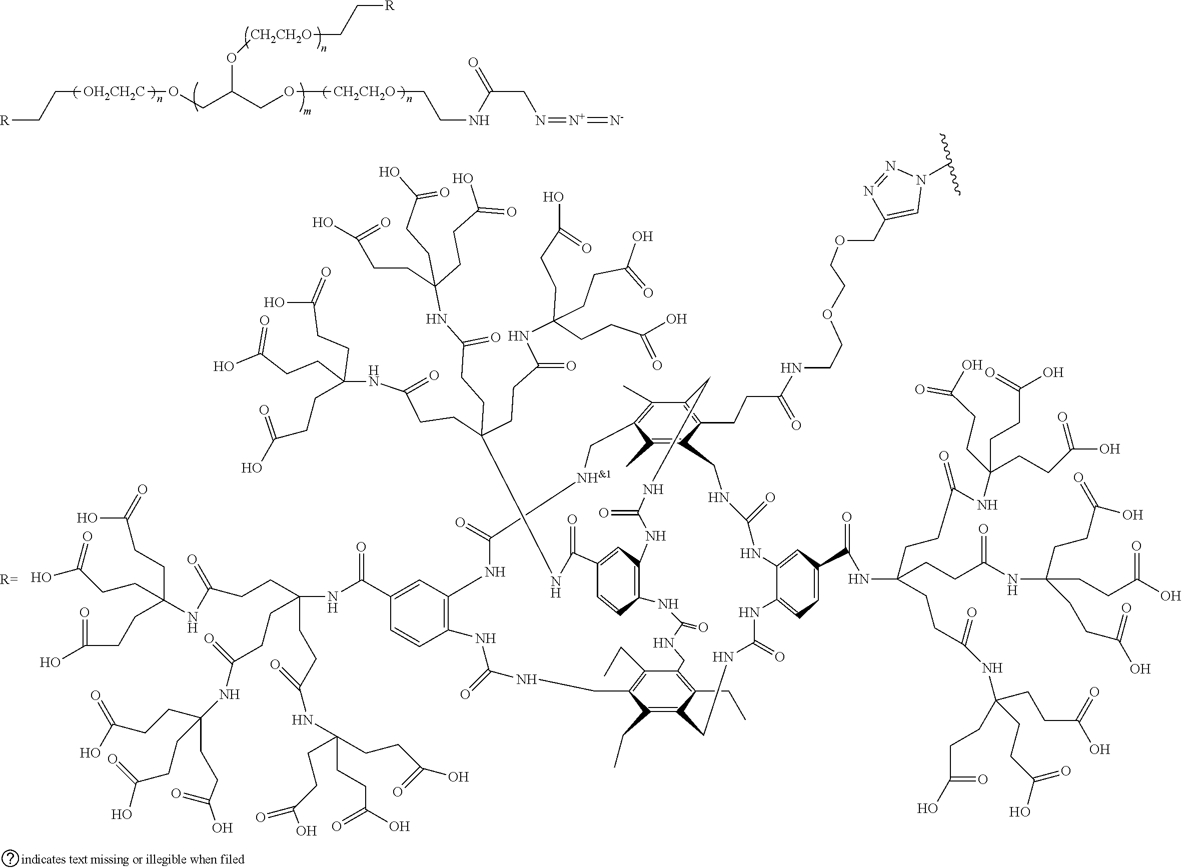

(where m is about 4 to 8, e.g. 6 and n is between about 100 and about 120 (e.g. about 110, 111 or 112)

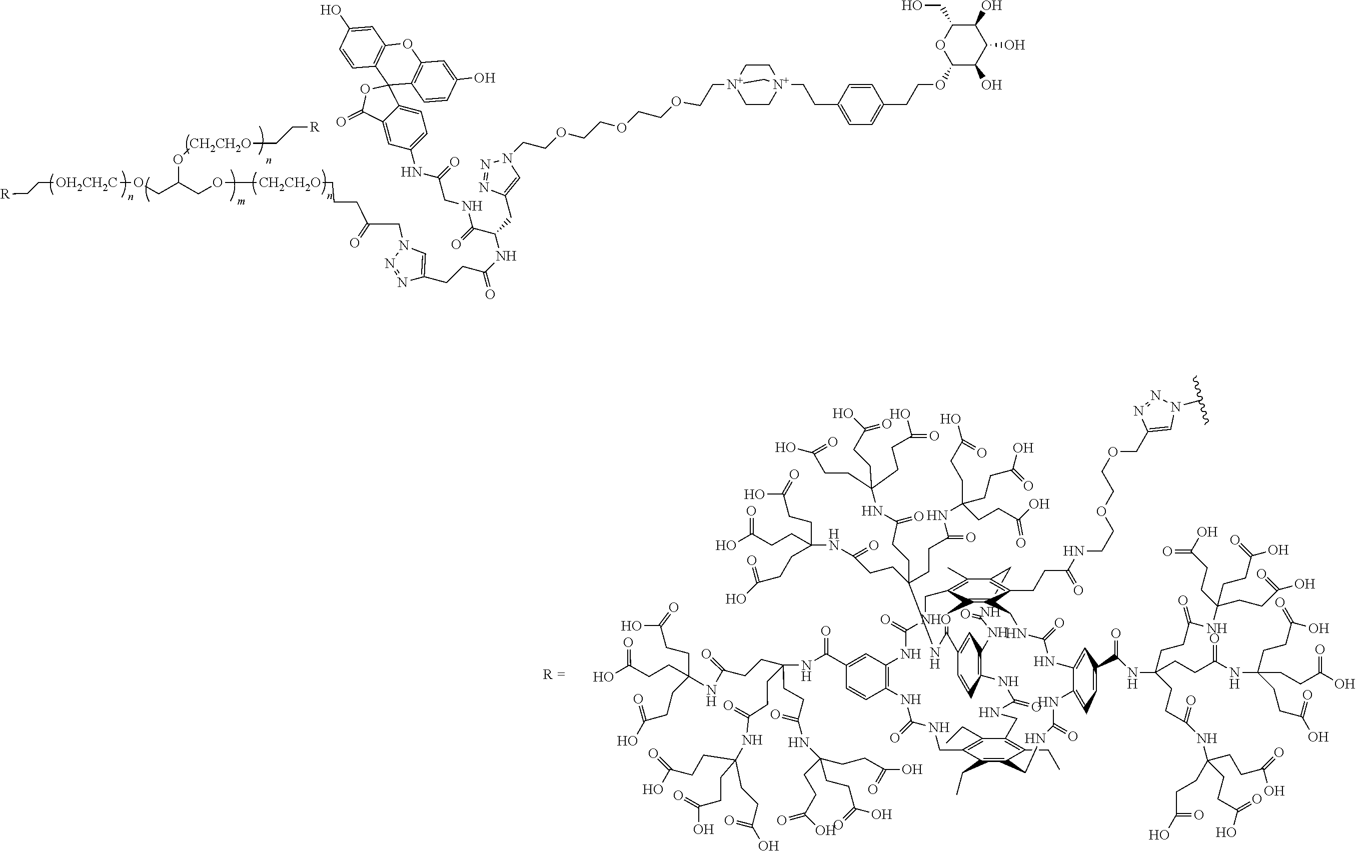

(where m is about 4 to 8, e.g. 6 and n is between about 100 and about 120 (e.g. about 110, 111 or 112)

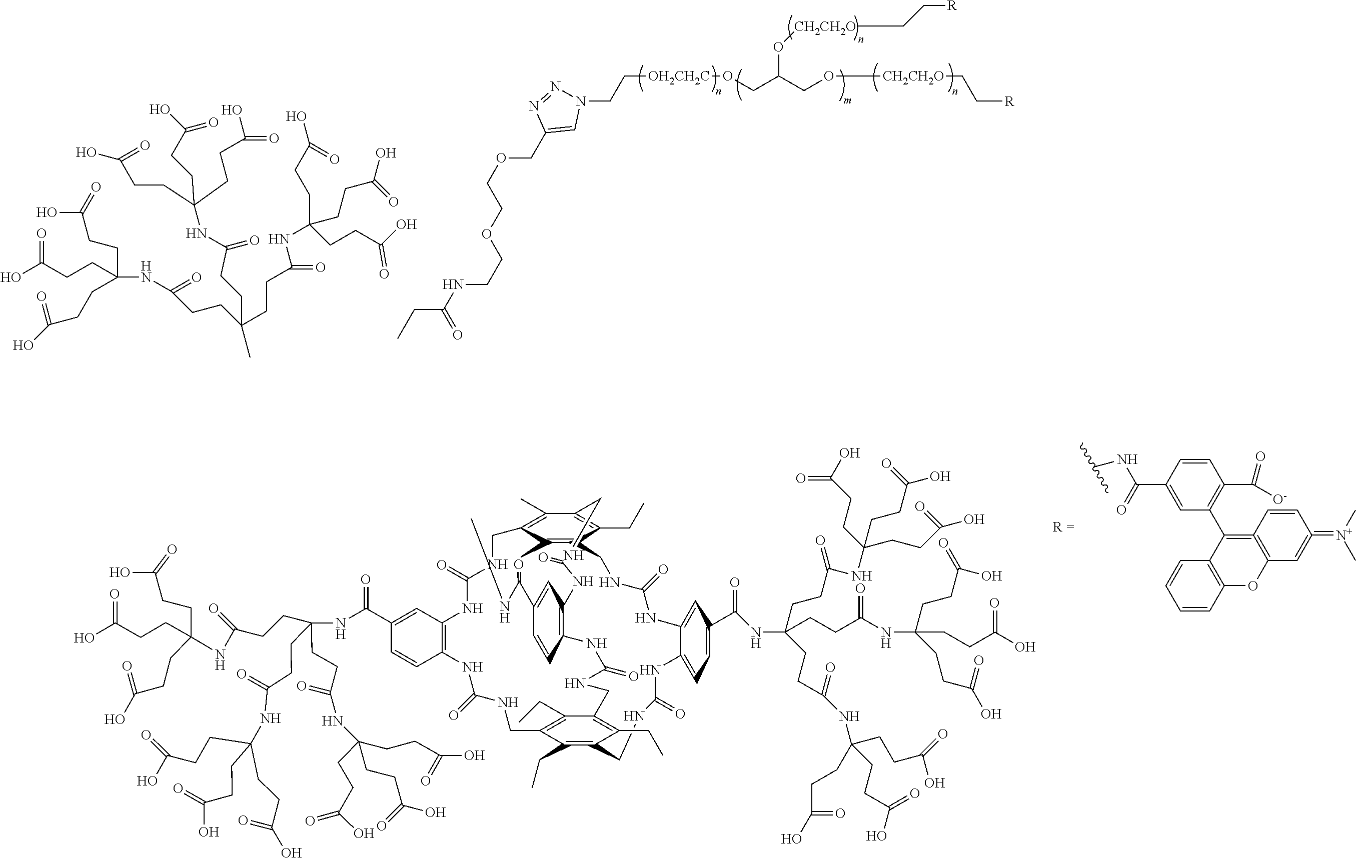

(where m is about 4 to 8, e.g. 6 and n is between about 100 and about 120 (e.g. about 110, 111 or 112)

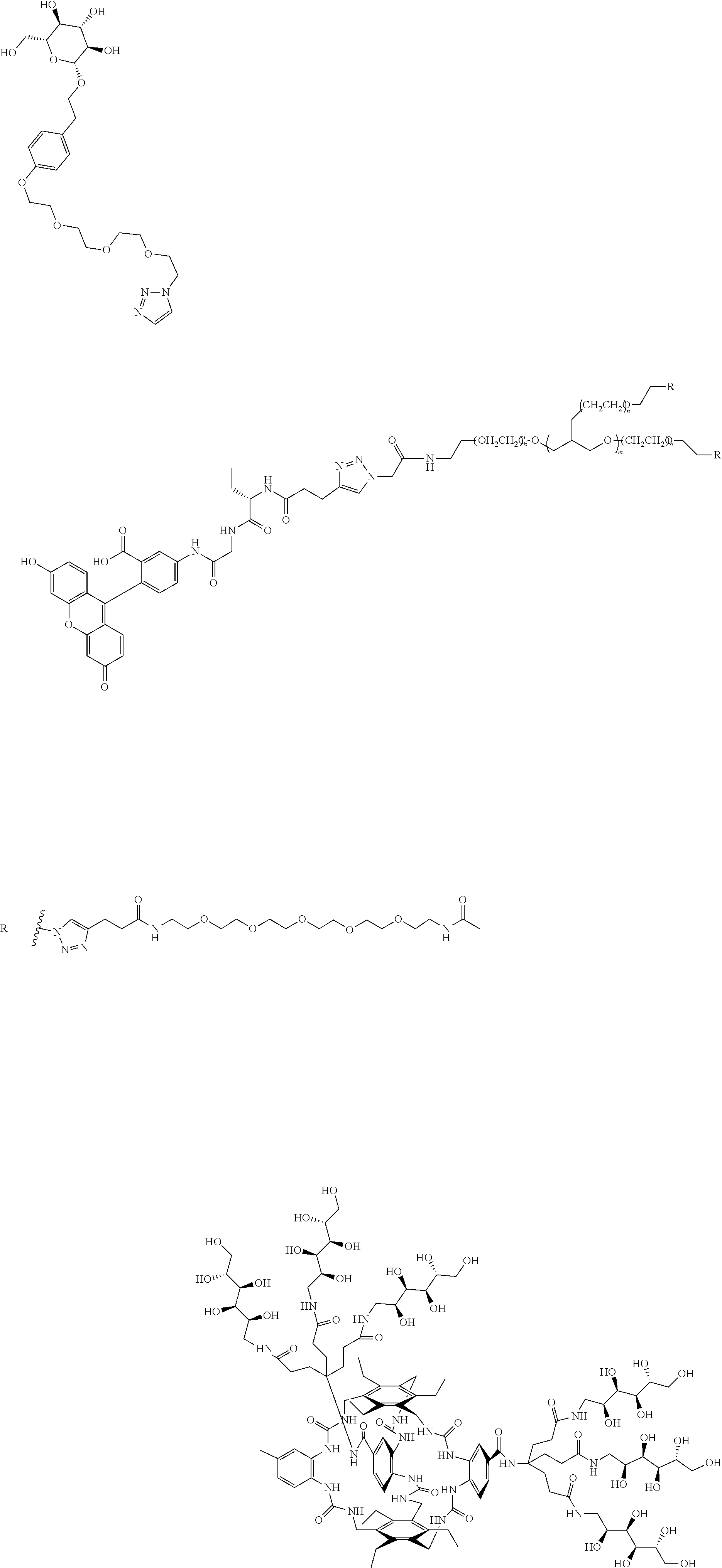

(where m is about 4 to 8, e.g. 6 and n is between about 100 and about 120 (e.g. about 110, 111 or 112)

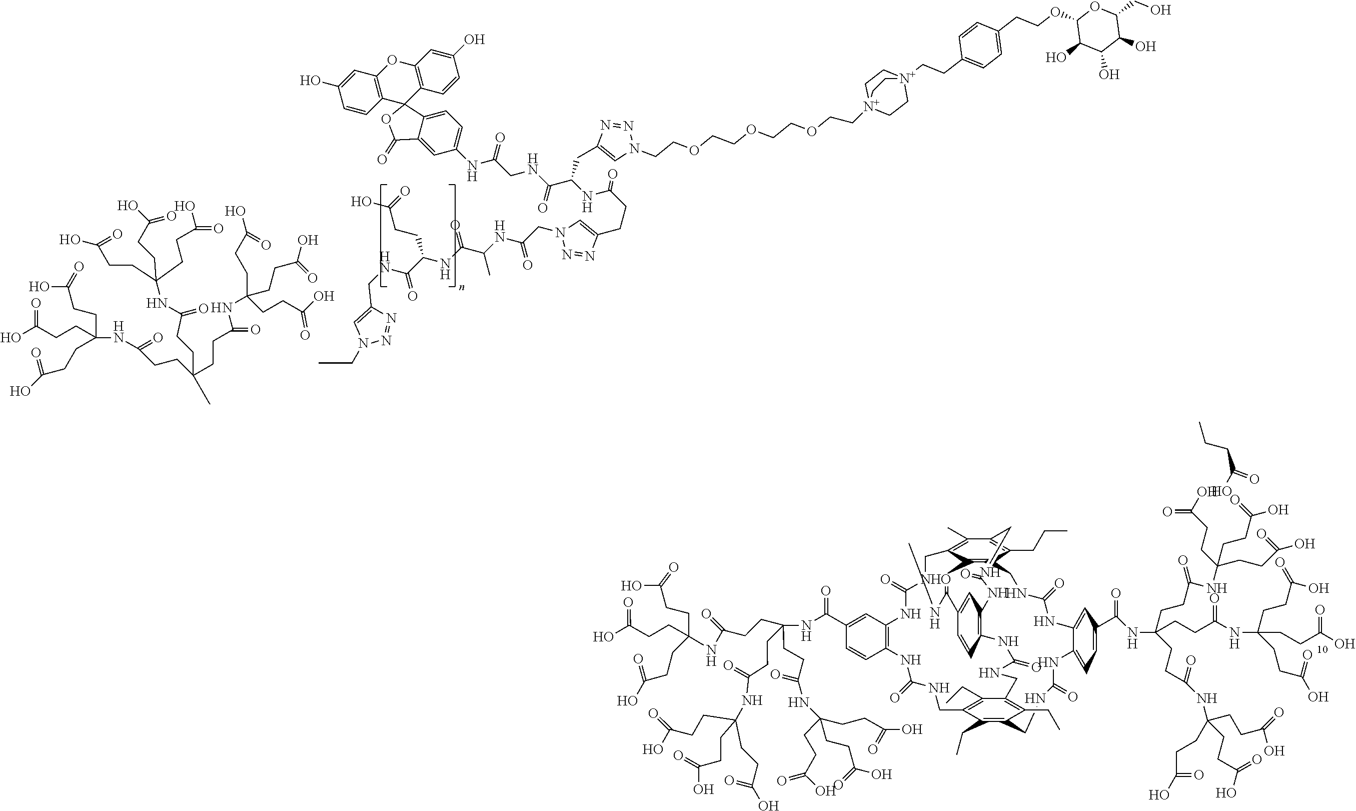

Where n is from1 40 to 160, such as 154

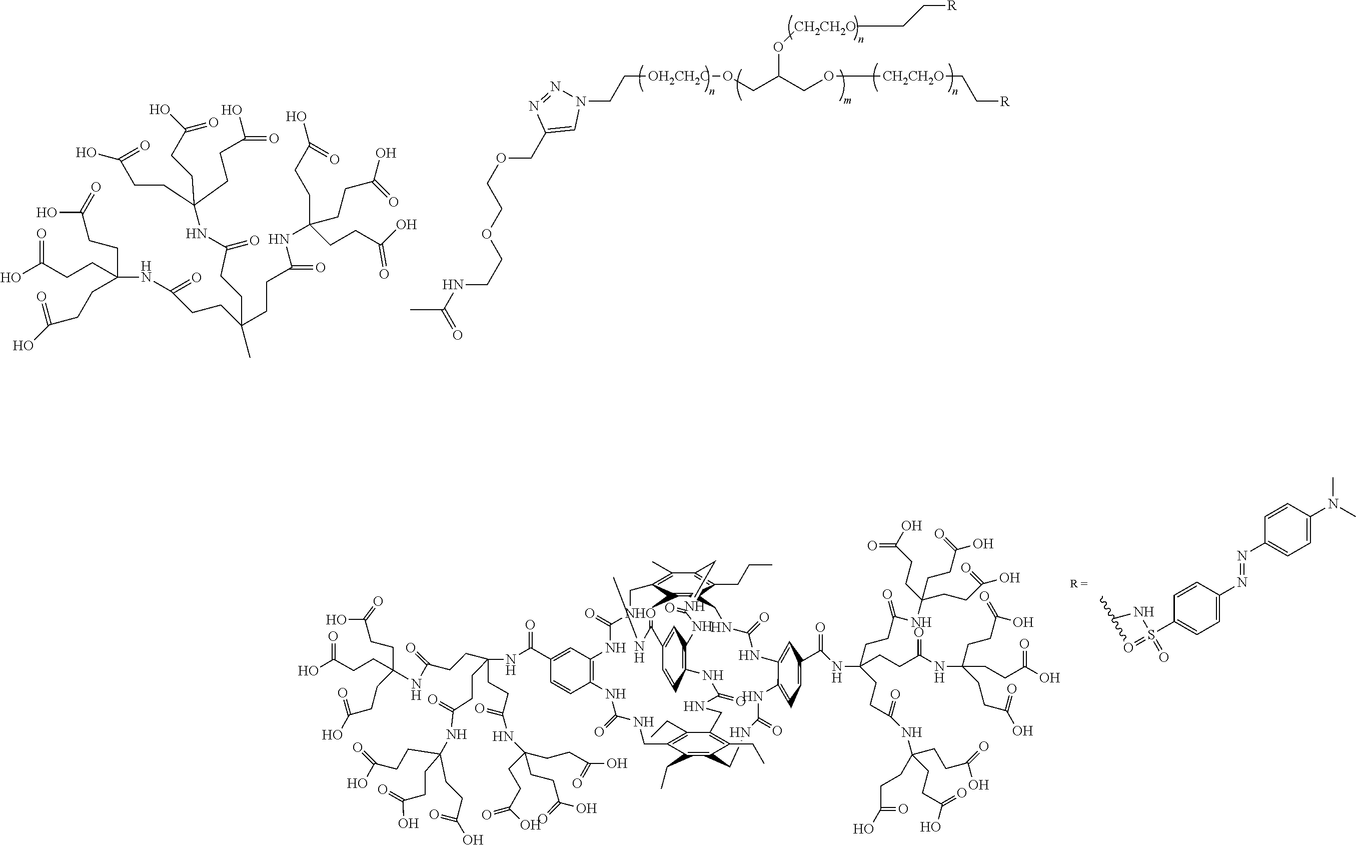

(where m is about 4 to 8, e.g. 6 and n is between about 100 and about 120 (e.g. about 110, 111 or 112)

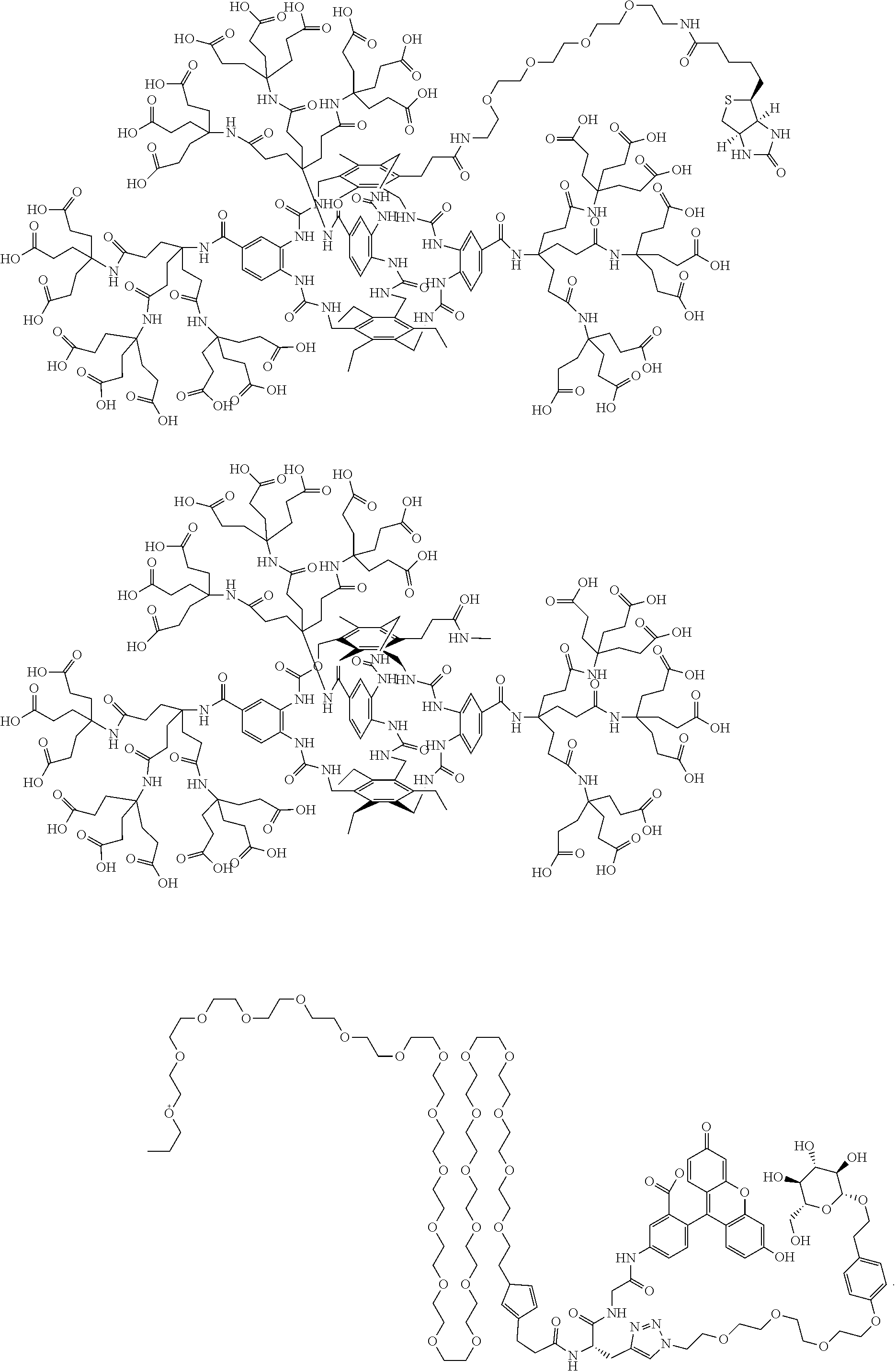

A suitable salt of the saccharide-binding compound is, for example, an acid-addition salt of the saccharide-binding compound which is sufficiently basic, for example, an acid-addition salt with, for example, an inorganic or organic acid, for example hydrochloric, hydrobromic, sulfuric, phosphoric, trifluoroacetic, formic, citric methane sulfonate or maleic acid. In addition, a suitable salt of the saccharide-binding compound which is sufficiently acidic is an alkali metal salt, for example a sodium or potassium salt, an alkaline earth metal salt, for example a calcium or magnesium salt, an ammonium salt or a salt with an organic base which affords an acceptable cation, for example a salt with methylamine, dimethylamine, trimethylamine, piperidine, morpholine or tris-(2-hydroxyethyl)amine.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E- and Z- isomers). It is to be understood that the present invention encompasses all optical, diastereoisomers and geometric isomers and mixtures thereof that are capable of saccharide recognition.

The present invention also encompasses compounds of the invention as defined herein which comprise one or more isotopic substitutions. For example, H may be in any isotopic form, including 1H, 2H(D), and 3H (T); C may be in any isotopic form, including 12C, 13C, and 14C; and O may be in any isotopic form, including 160 and180; and the like.

It is also to be understood that certain compounds of the Formula (I) (and sub-formulae) may exist in solvated as well as unsolvated forms such as, for example, hydrated forms. It is to be understood that the invention encompasses all such solvated forms that are capable of saccharide recognition.

It is also to be understood that certain compounds of the Formula (I) (and sub-formulae) may exhibit polymorphism, and that the invention encompasses all such forms that are capable of saccharide recognition.

Compounds of the Formula (I) (and sub-formulae) may exist in a number of different tautomeric forms and references to compounds of the Formula (I) (and sub-formulae) include all such forms. For the avoidance of doubt, where a compound can exist in one of several tautomeric forms, and only one is specifically described or shown, all others are nevertheless embraced by Formula I. Examples of tautomeric forms include keto-, enol-, and enolate-forms, as in, for example, the following tautomeric pairs: keto/enol, imine/enamine, amide/imino alcohol, amidine/amidine, nitroso/oxime, thioketone/enethiol, and nitro/aci-nitro.

Compounds of the Formula (I) (and sub-formulae) containing an amine function may also form N-oxides. A reference herein to a compound of the Formula I that contains an amine function also includes the N-oxide. Where a compound contains several amine functions, one or more than one nitrogen atom may be oxidised to form an N-oxide. Particular examples of N-oxides are the N-oxides of a te/tiary amine or a nitrogen atom of a nitrogen-containing heterocycle. N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (mCPBA), for example, in an inert solvent such as dichloromethane.

Though the present invention may relate to any compound or particular group of compounds defined herein by way of optional, preferred or suitable features or otherwise in terms of particular embodiments, the present invention may also relate to any compound or particular group of compounds that specifically excludes said optional, preferred or suitable features or particular embodiments.

### The Detectable Reporter

The detectable reporter provides a detectable signal based on the detection or change of the target saccharide in an aqueous environment. The detectable reporter of the fourth aspect is included in the saccharide-binding compound and is described above.

The detectable reporter of the first and third aspects is a displaceable cavity-binding ligand including a fluorescent reporter moiety and a cavity-binding moiety for binding the cavity of the saccharide-binding compound. The displaceable cavity-binding ligand of the third aspect is attached to the semi-solid or solid support.

The detectable reporter of the second aspect is a displaceable cavity-binding ligand including a redox moiety and a cavity-binding moiety for binding the cavity of the saccharide-binding compound.

The ligand may be in a first state where the cavity-binding moiety is associated with (e.g. bound in) the cavity of a saccharide-binding compound as defined herein and a second state where the cavity-binding moiety is not associated (e.g. displaced from) with the cavity of a saccharide-binding compound as defined herein.

Without wishing to be bound by theory, the ligand will typically be in equilibrium between the first and second state. The equilibrium between the first and the second state may be influenced by the presence or absence, and in particular the concentration, of the target saccharide. In some embodiments, the first state of the ligand is favoured at a first concentration of the target saccharide and the second state of the ligand is favoured at a second concentration of the target saccharide, and the first concentration is a lower concentration of target saccharide than the second concentration.

### Fluorescent Reporter Moiety

The fluorescent reporter moiety typically provides a detectable emission in one or both of the first or second state. In particular embodiments, the displaceable cavity-binding ligand emits different emissions in the first and second states, such that a change from the first to second and/or second to first state provides a detectable change in the emission from the ligand.

In some embodiments, the ligand has an emission wavelength in the range of about 300 nm to about 1000 nm. In further embodiments the ligand has an emissions wavelength in the range of about 300 nm to about 800 nm. In more particular embodiments, the ligand has an emissions wavelength in the range of about 500 nm to about 700 nm.

The fluorescent reporter moiety may include a fluorophore.

In some embodiments, fluorescent reporter moiety is selected from xanthene dyes or derivatives (such as fluorescein, rhodamine, Oregon green, eosin, and Texas red); cyanine dyes or derivatives (such as cyanine, indocarbocyanine, oxacarbocyanine, thiacarbocyanine, and merocyanine); squaraine dyes or derivatives and ring-substituted squaraines (such as Seta and Square dyes); squaraine rotaxane dyes or derivatives (such as Tau dyes); naphthalene dyes or derivatives (such as dansyl and prodan dyes or derivatives); coumarin dyes or derivatives; oxadiazole dyes or derivatives (such aspyridyloxazole, nitrobenzoxadiazole and benzoxadiazole); anthracene dyes or derivatives (such as anthraquinones, e.g. DRAQ5, DRAQ7 and CyTRAK Orange); pyrene dyes or derivatives (such as cascade blue); oxazine dyes or derivatives (such as Nile red, Nile blue, cresyl violet, oxazine 170); acridine dyes or derivatives (such as proflavin, acridine orange, acridine yellow); arylmethine dyes or derivatives (such as auramine, crystal violet, malachite green); tetrapyrrole dyes or derivatives (such as porphin, phthalocyanine, bilirubin); dipyrromethene dyes or derivatives (such as BODIPY, aza-BODIPY), metal coordination complexes or organometallic fluorophores (such as [Ru(bpy)₃]²⁺ (“Ru-bpy”) and Ln³⁺ chelate complexes), fluorescent dyes in the Alexa Fluor™ series (such as AF488™ and AF594™) and quinoline dyes or derivatives.

In further embodiments, the fluroescent reporter moiety is selected from a xanthene derived dye, a cyanine derived dye, a napthalene derived dye, a pyrene derived dye, a pyranine derived dye, metal coordination complexes or organometallic fluorophores and a fluorescent dye in the Alexa Fluor™ series.

In particular embodiments, the fluorescent reporter moiety is selected from a xanthene derived dye, such as fluorescein and rhodamine, [Ru(bpy)₃]²+, a Ln³⁺ chelate complex and a fluorescent dye in the Alexa Fluor™ series, such as AF488™ and AF™. Specific examples of the fluorophore include but are not limited to AF430, AF488, AF594, BODIPY, Cyanine, Cy3, Cy7.5, EDANS, Fluorescein, Pyranine, Rhodamine 6G, Rhodamine B, Ru(BIPY)3, TAMRA, Phenazine.

### Redox Reporter Moiety

The redox reporter moiety is a moiety capable of reversible oxidation and/or reduction. In this way, a change in the reduction potential and/or oxidation potential of the displaceable cavity-binding ligand can be detected over time and/or with a changing composition (e.g. target saccharide concentration) in the aqueous solution of the system of the second aspect. In some embodiments, the current from the oxidation and/or the reduction may be used to detect a change in the system either instead of or in addition to the reduction and/or oxidation potentials. The reduction potential, oxidation potential and current properties may be measured, for example, by cyclic voltammetry.

The redox reporter moiety may be selected from the group consisting of methylene blue, anthraquinone, ferrocene or derivatives thereof.

### Cavity-Binding Moiety

The cavity-binding moiety is suitable for binding the cavity of the saccharide-binding compound. The cavity-binding moiety may be selected so that cavity-binding moiety is displaced from the cavity of the saccharide-binding compound when the saccharide sensing system is contacted with the target saccharide-containing aqueous environment.

In some embodiments, the cavity-binding moiety includes a hydrogen-bond acceptor. In particular embodiments, the cavity-binding moiety includes a saccharide, such as a glucoside or a glycoside. The saccharide may be linked to the rest of the cavity-binding substituent group by a beta-linkage. In more particular embodiments, the cavity-binding moiety includes a glucoronide. In specific embodiments, the cavity-binding moiety is salidroside.

In addition to the cavity-binding moiety and fluorescent reporter moiety or redox moiety, the cavity-binding ligand may further include a ligand membrane blocking group (LMBG) and/or a terminal group T_{L}.

### Ligand Membrane Blocking Group LMBG

In some embodiments, the displaceable cavity-binding ligand includes a ligand membrane blocking group (LMBG). In these embodiments, the system may include a molecular weight cut off membrane and the ligand membrane blocking group LMBG has a molecular weight sufficient to increase the molecular weight of the displaceable cavity-binding ligand above the molecular weight cut off of the molecule weight cut off membrane.

In some embodiments, the ligand membrane blocking group LMBG has a molecular weight sufficient to increase the molecular weight of the displaceable cavity-binding ligand above 1,000 daltons (1 kDa). In particular embodiments, the ligand membrane blocking group LMBG has a molecular weight sufficient to increase the molecular weight of the displaceable cavity-binding ligand above about 2,000 daltons (2 kDa), above about 5,000 daltons (5 kDa), above about 10,000 daltons (10 kDa) or above about 30,000 daltons (kDa).

In more particular embodiments, the ligand membrane blocking group LMBG has a molecular weight of at least about 1,000 daltons (1 kDa), at least about 2,000 daltons (2 kDa), at least about 5,000 daltons (5 kDa), at least about 10,000 daltons (10 kDa) or at least about 30,000 daltons (30 kDa).

The ligand membrane blocking group LMBG may be a water soluble polymer, such as water soluble polysaccharides (such as dextran), polyacrylamides, polyethylene glycol, peptides (such as oligopeptides or polypeptides) and nucleic acids (such as DNA or RNA).

In particular embodiments, the ligand membrane blocking group is a water soluble polysaccharide, polyacrylamide or polyethylene glycol having a molecular weight of at least about 30 kDa. In more particular embodiments, the ligand membrane blocking group is a dextran or polyethylene glycol having a molecular weight of at least about 30 kDa. In specific embodiments, the ligand membrane blocking group is a dextran of polyethylene glycol having a molecular weight of in the range of about 30 kDa to about 50 kDa, such as about 40 kDa.

### Terminal Group T_{L}

The terminal group is a monovalent group for terminating part of the displaceable cavity-binding ligand. In some embodiments, the terminal group is capable of reacting with a semi-solid or solid support or precursors of a semi-solid or solid support (such as monomers or oligomers). In other embodiments, the terminal group may be a solubilizing group for increasing the solubility of the displaceable cavity-binding ligand in a solvent, such as water.

In some embodiments, T_{L} may be selected from the group consisting of X, OH, SH, SOX, SO₂X, SO₃H, N(R^{x4})H, C(O)H, C(O)OH, OC(O)H, C(O)N(R^{x4})H, N(R^{x4})C(O)H, N(R^{x4})C(O)N(R^{x5})H, N(R^{x4})C(O)OH, OC(O)N(R^{x4})H, S(O)₂N(R^{x4})H, N(R^{x4})SO₂H, —N₃, (1-20C)alkyl, (1-20C)alkyl oxide, (1-20C)alkene and (1-20C)alkyne, aryl, (3-10C)cycloalkyl, (3-10C)cycloalkene, heteroaryl, heterocyclyl, peptides (such as oligopeptides or polypeptides) and nucleic acids (such as DNA or RNA), wherein X is a halogen; and R^{x4} and R^{x5} are each independently selected from hydrogen and (1-4C)alkyl.

In particular embodiments, the terminal group is selected from —C═CH; —CH═CH₂, bicyclo[6.1.0]nonynl, -tBu, —N₃, —NH₂.

Ligand linker units, L_{Lunit} The displaceable cavity-binding ligand may include one or more ligand linker units (L_{Lunit}) linking the cavity-binding moiety and fluorescent reporter moiety or the redox moiety. The ligand linker units may also link the terminal group T_{L} and/or ligand membrane blocking group (when present) to rest of the ligand. In some embodiments, the ligand linker units may be units formed from a “click” reaction.

In some embodiments, the cavity-binding moiety is linked by the ligand linker unit(s) to only the fluorescent reporter moiety or the redox moiety. In these embodiments, the ligand linker units (L_{Lunit}) may consist of one or more linear linkers independently selected from the group consisting of O, S, SO, SO₂, N(R^{x4}), C(O), C(O)O, OC(O), C(O)N(R^{x4}), N(R^{x4})C(O), N(R^{x4})C(O)N(R^{x5}), N(R^{x4})C(O)O, OC(O)N(R^{x4}), S(O)₂N(R^{x4}), N(R^{x4})SO₂, amino acids, (1-20C)alkylene, (1-20C)alkylene oxide, (1-20C)alkenyl, (1-20C)alkynyl, aryl, (3-10C)cycloalkyl, (3-10C)cycloalkenyl, heteroaryl and heterocyclyl, wherein R^{x4} and R^{x5} are each independently selected from hydrogen and (1-4C)alkyl.

In these embodiments, the displaceable cavity binding ligand may include from 1 to 25 linear linker units and each linear linker unit is independently selected from the group consisting of 0, N(R^{x4}), C(O), C(O)O, C(O)N(R^{x4}), N(R^{x4})C(O)N(R^{x5}), (O1-4Calkyl)_{g}, C(O)1-4Calkyl, N(R^{x4})1-4C alkyl, C(O)N(R^{x4})1-4C alkyl, N(R^{x4})C(O)1-4C alkyl, N(R^{x4})C(O)N(R^{x4})1-4C alkyl, -(amino acid)-, triazolyl-1-4Calkyl, 1-4Calkyl-triazolyl-1-4Calkyl, wherein g is an integer selected from 1 to 100, j is an integer selected from 1 to 200 and R^{x4} and R^{x5} are each independently selected from hydrogen and (1-4C)alkyl. In some embodiments, g is an integer from 1 to 50, 1 to 25, 1 to 20, 1 to 15 or 1 to 5. In some embodiments, g is an integer from 2 to 50, 2 to 25, 2 to 20, 2 to 15 or 2 to 5. In some embodiments, j is an integer from 100 to 200, from 125 to 175 or from 140 to 160

In some embodiments, the cavity-binding moiety includes a terminal group T_{L} and/or ligand membrane blocking group, and either the terminal group T_{L} and/or ligand membrane blocking group being substituted onto one of the linker units linking the cavity-binding moiety to the fluorescent reporter moiety or the redox moiety, or one or more of the linker units linking the cavity-binding moiety to the fluorescent reporter moiety or the redox moiety is replaced with a branched linker, wherein each branched linker is a linear linker as described herein substituted with one or more linear linkers as described herein and linking the branched linker to terminal group T_{L} and/or ligand membrane blocking group.

For example, the displaceable cavity-binding ligand may include one or more linear linker units from the cavity-binding moiety up to a linker unit substituted with a terminal group T_{L} and/or a ligand membrane blocking group and then further linear linker units from the substituted linker unit to the other of the fluorescent reporter moiety or the redox moiety. Alternatively, the displaceable cavity-binding ligand may include one or more linear linker units from the cavity-binding moiety up to a branched linker unit, the branched linker unit being further attached to two sets of one or more linker units, each set of linker units linking the branched linker unit to the fluorescent reporter moiety or the redox moiety and to one of terminal group T_{L} and/or a ligand membrane blocking group. In other words, one set of linker units is attached to the branched linker unit and the fluorescent reporter moiety or the redox moiety and the other set of linker units is attached to the branched linker unit and terminal group T_{L} and/or a ligand membrane blocking group.

When the ligand is attached to a semi-solid or solid support, the ligand may include one or more linker units as described herein linking the semi-solid or solid support to the rest of the ligand. The linker units may link the semi-solid or solid support to the cavity-binding moiety, fluorescent reporter moiety or the redox moiety, terminal group T_{L}(when present), ligand membrane blocking group (when present) or any linker units in the rest of the ligand as described herein. For example, the ligand may have one or more linker units as described herein linking the cavity-binding moiety and the fluorescent reporter moiety or the redox moiety, these linker units including a branched linker as described herein, and the ligand including one or more linker units as described herein linking the branched linker to semi-solid or solid support.

In particular embodiments, the ligand includes one or more linker units as described herein linking the cavity-binding moiety and the fluorescent reporter moiety or the redox moiety, these linker units including a branched linker as described herein, and the ligand including one or more linker units linking the branched linker to one of the terminal group TL, a ligand membrane blocking group and, the semi-solid or solid support.

In particular embodiments, the displaceable cavity ligand has the structure selected from the group consisting of:

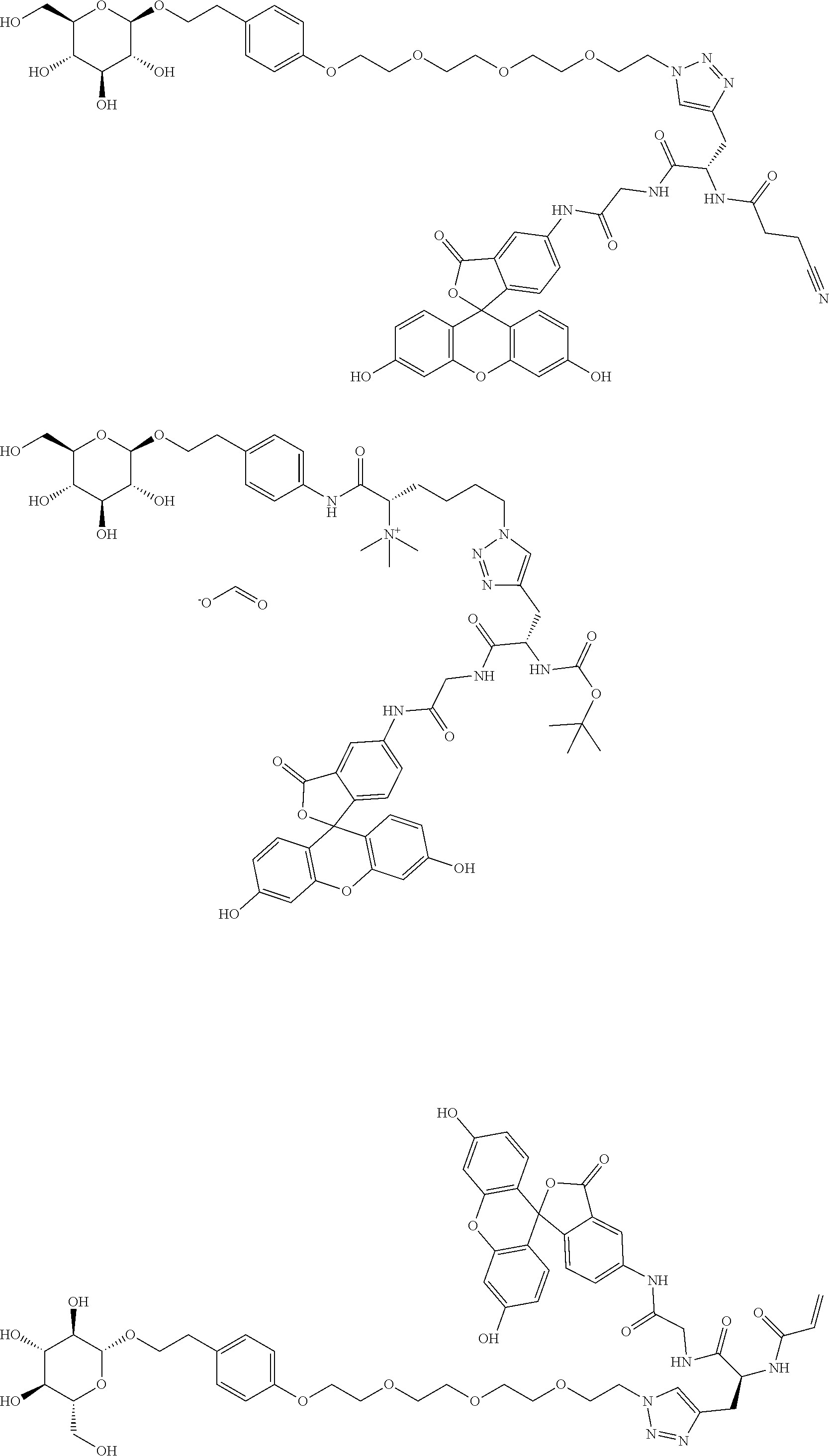

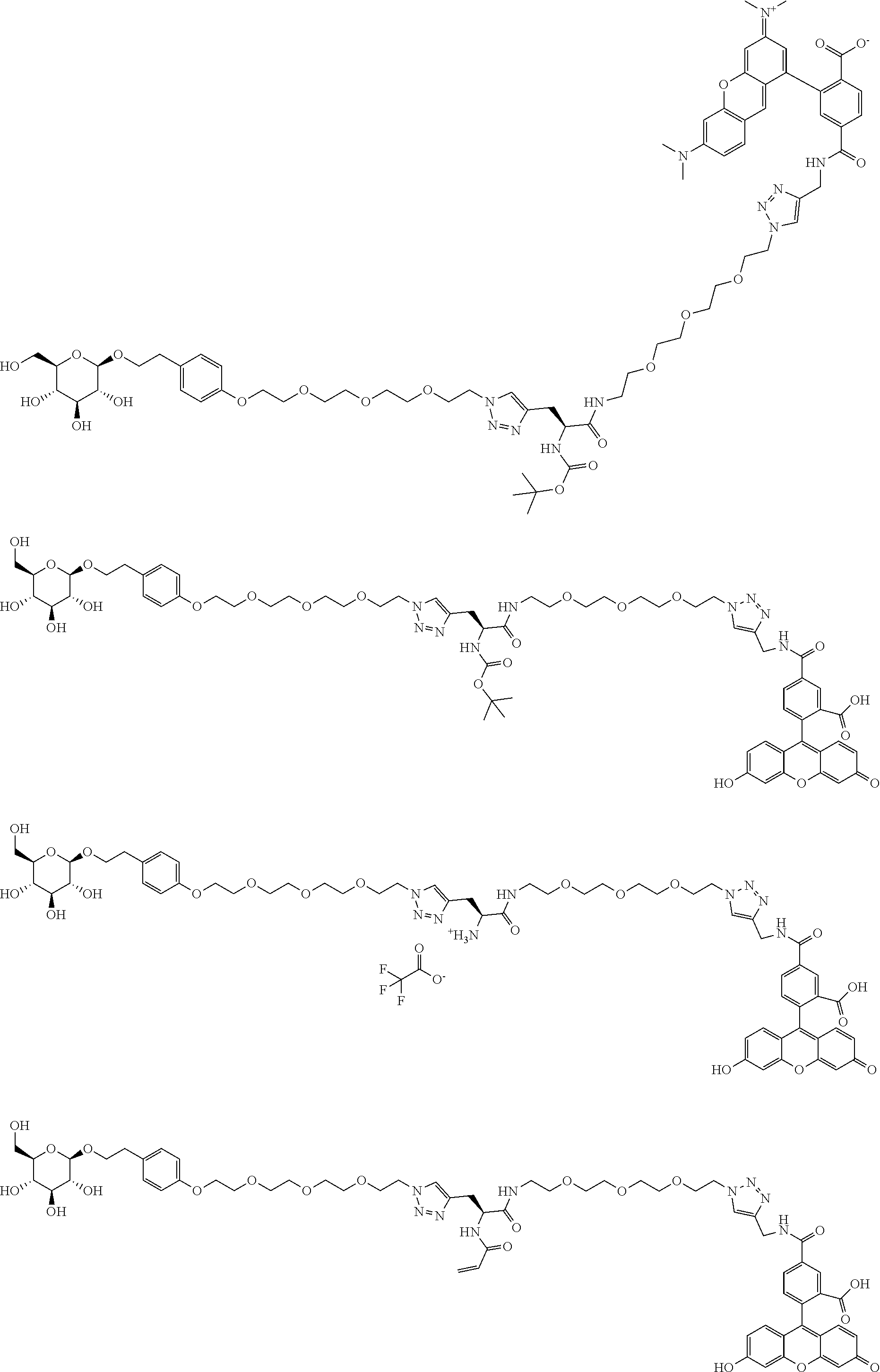

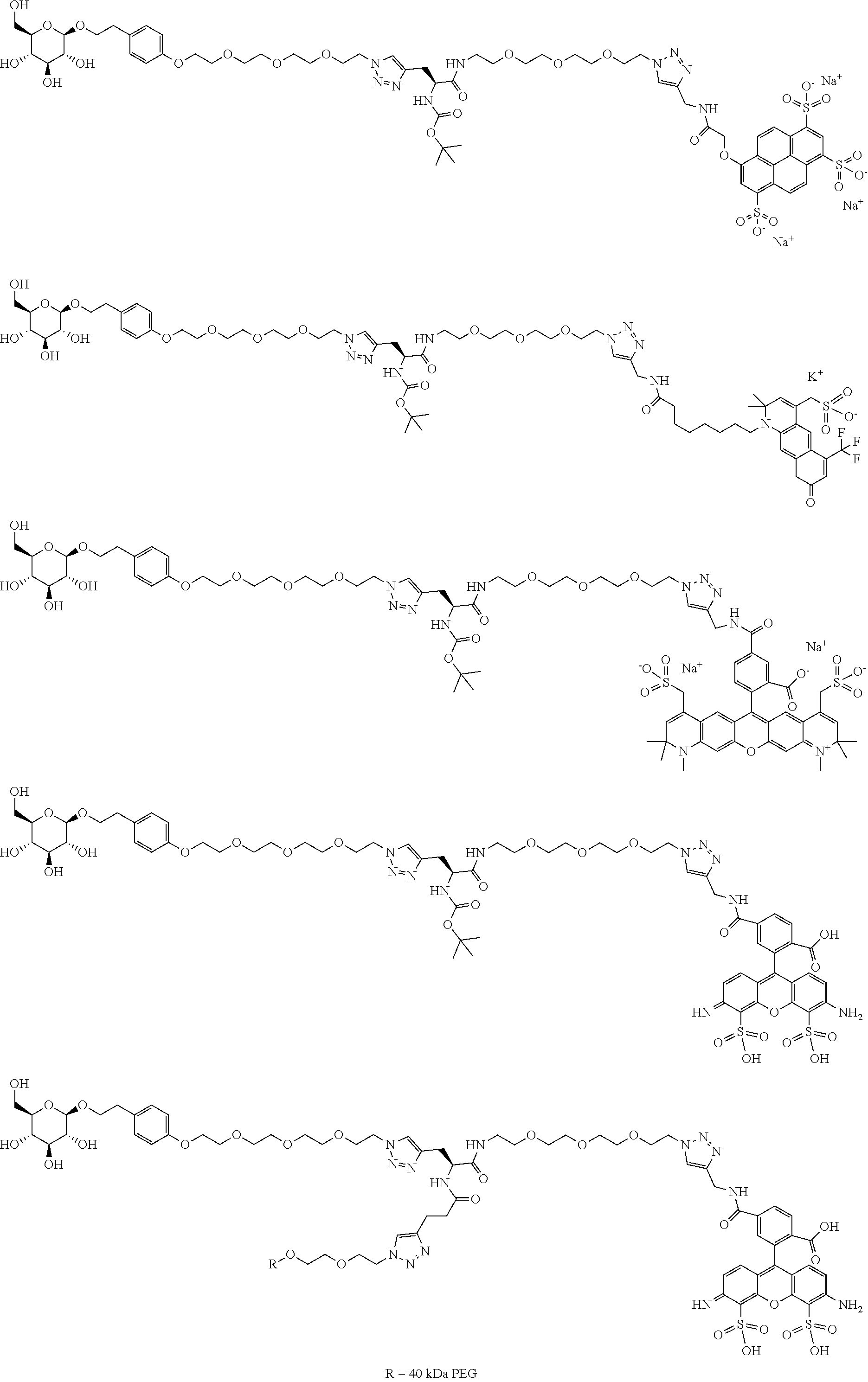

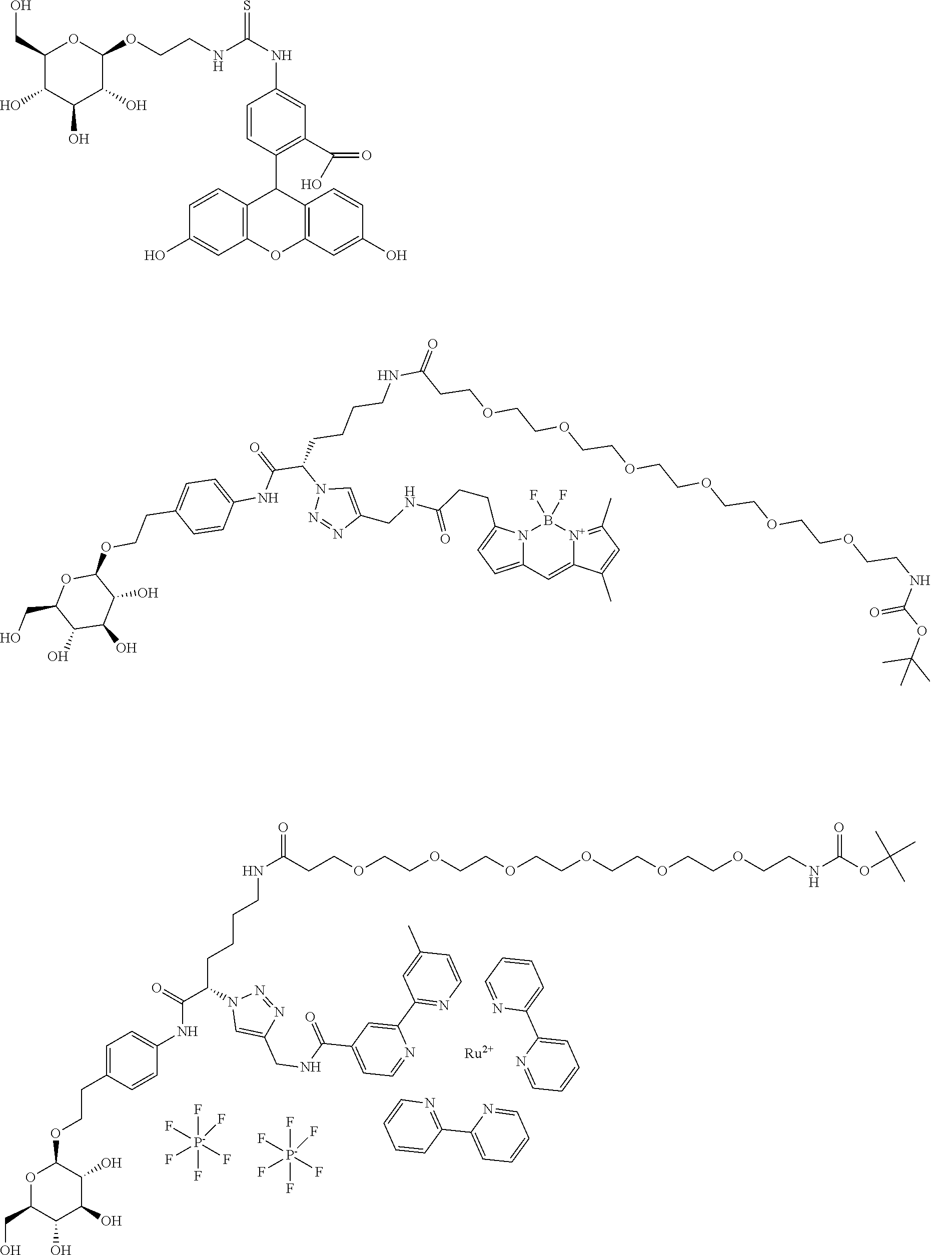

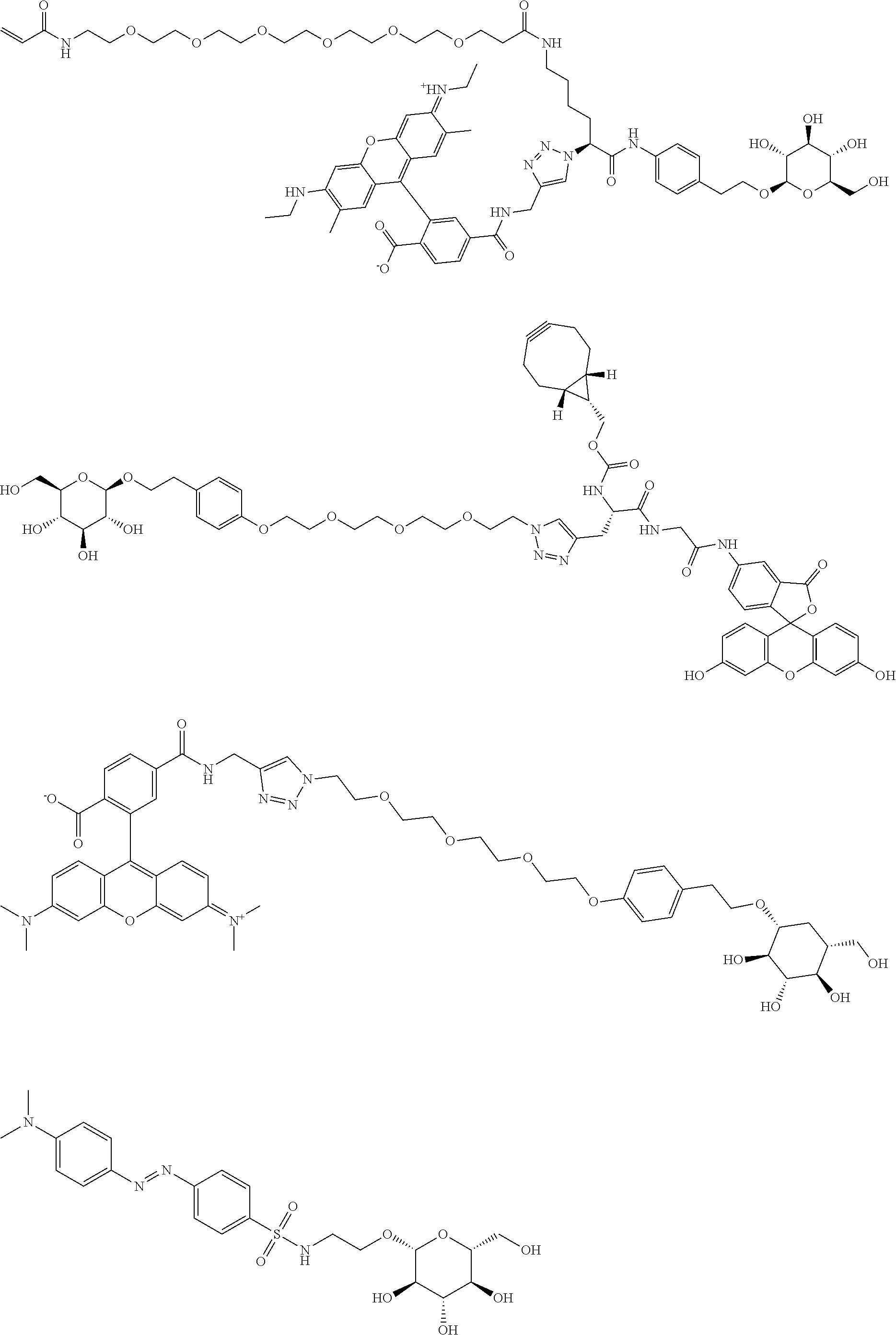

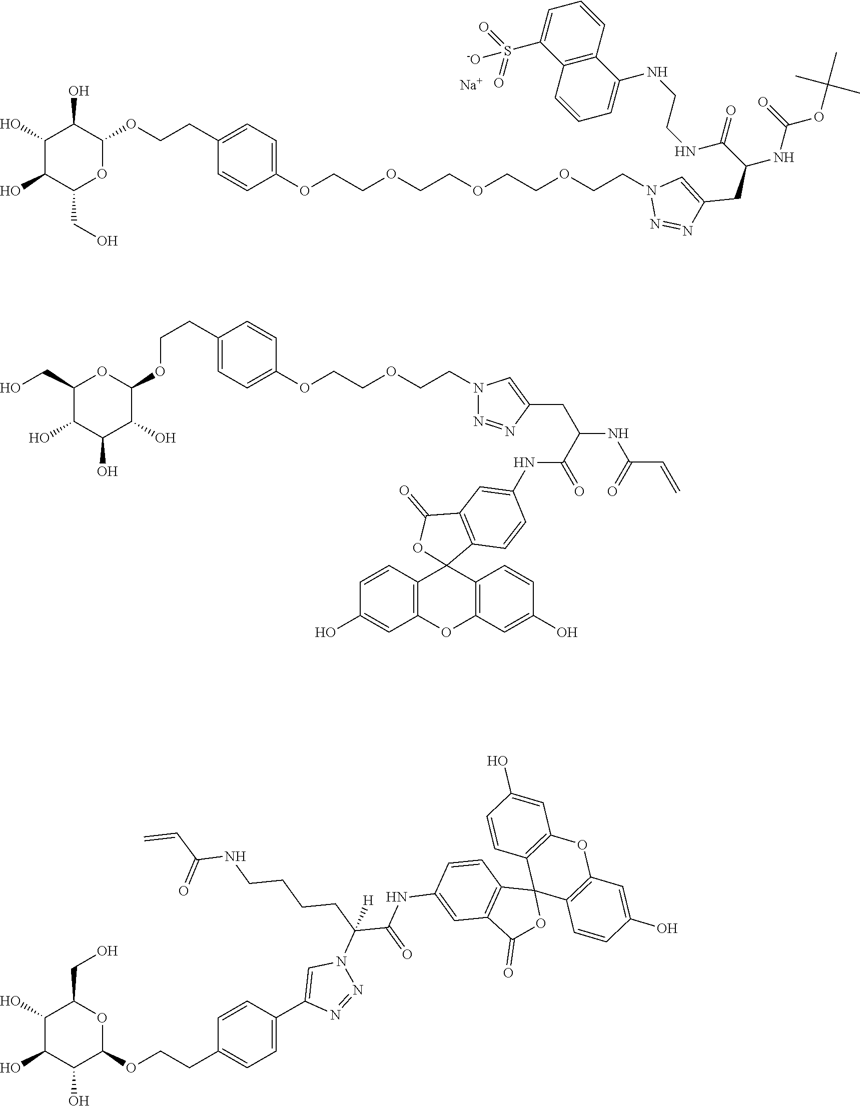

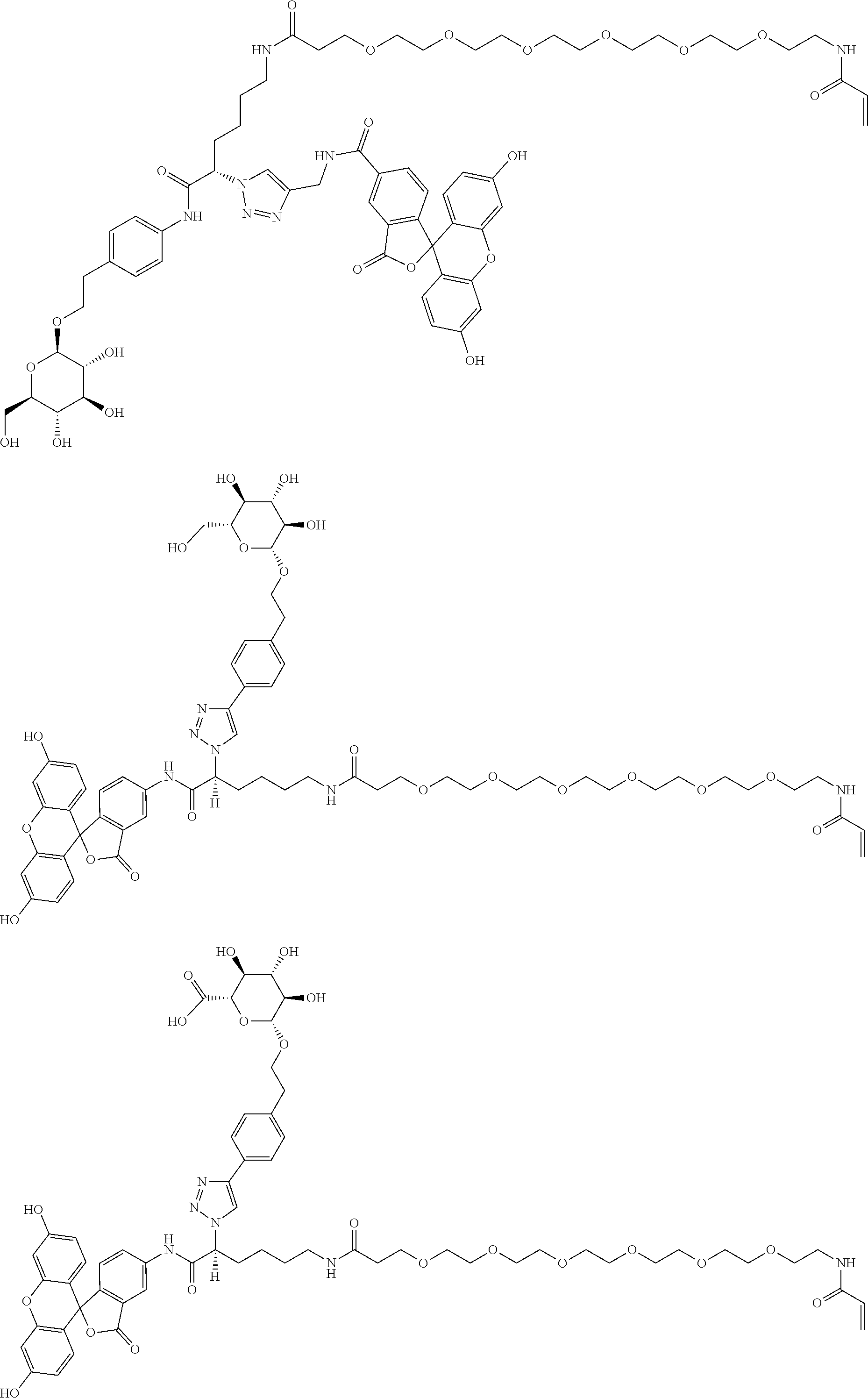 [SMILES: C=CC(=O)NCCOCCOCCOCCOCCOCCOCCC(=O)NCCCC[C@@H](C(=O)Nc1ccc(CCO[C@@H]2O[C@H](CO)[C@@H](O)[C@H](O)[C@H]2O)cc1)n1cc(CNC(=O)c2ccc3c(c2)C(=O)OC32c3ccc(O)cc3Oc3cc(O)ccc32)nn1 ; C=CC(=O)NCCOCCOCCOCCOCCOCCOCCC(=O)NCCCC[C@@H](C(=O)Nc1ccc2c(c1)C(=O)OC21c2ccc(O)cc2Oc2cc(O)ccc21)n1cc(-c2ccc(CCO[C@@H]3O[C@H](CO)[C@@H](O)[C@H](O)[C@H]3O)cc2)nn1 ; C=CC(=O)NCCOCCOCCOCCOCCOCCOCCC(=O)NCCCC[C@@H](C(=O)Nc1ccc2c(c1)C(=O)OC21c2ccc(O)cc2Oc2cc(O)ccc21)n1cc(-c2ccc(CCO[C@@H]3O[C@H](C(=O)O)[C@@H](O)[C@H](O)[C@H]3O)cc2)nn1]

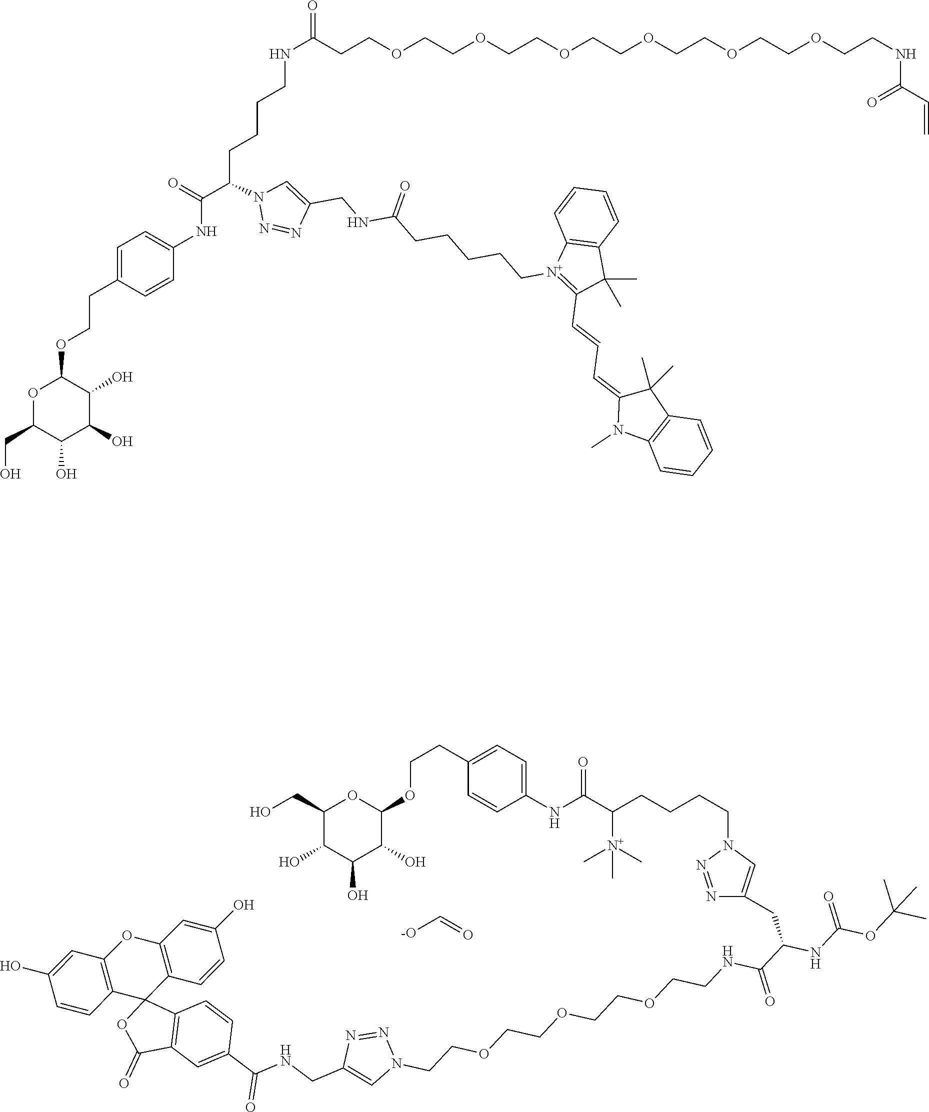

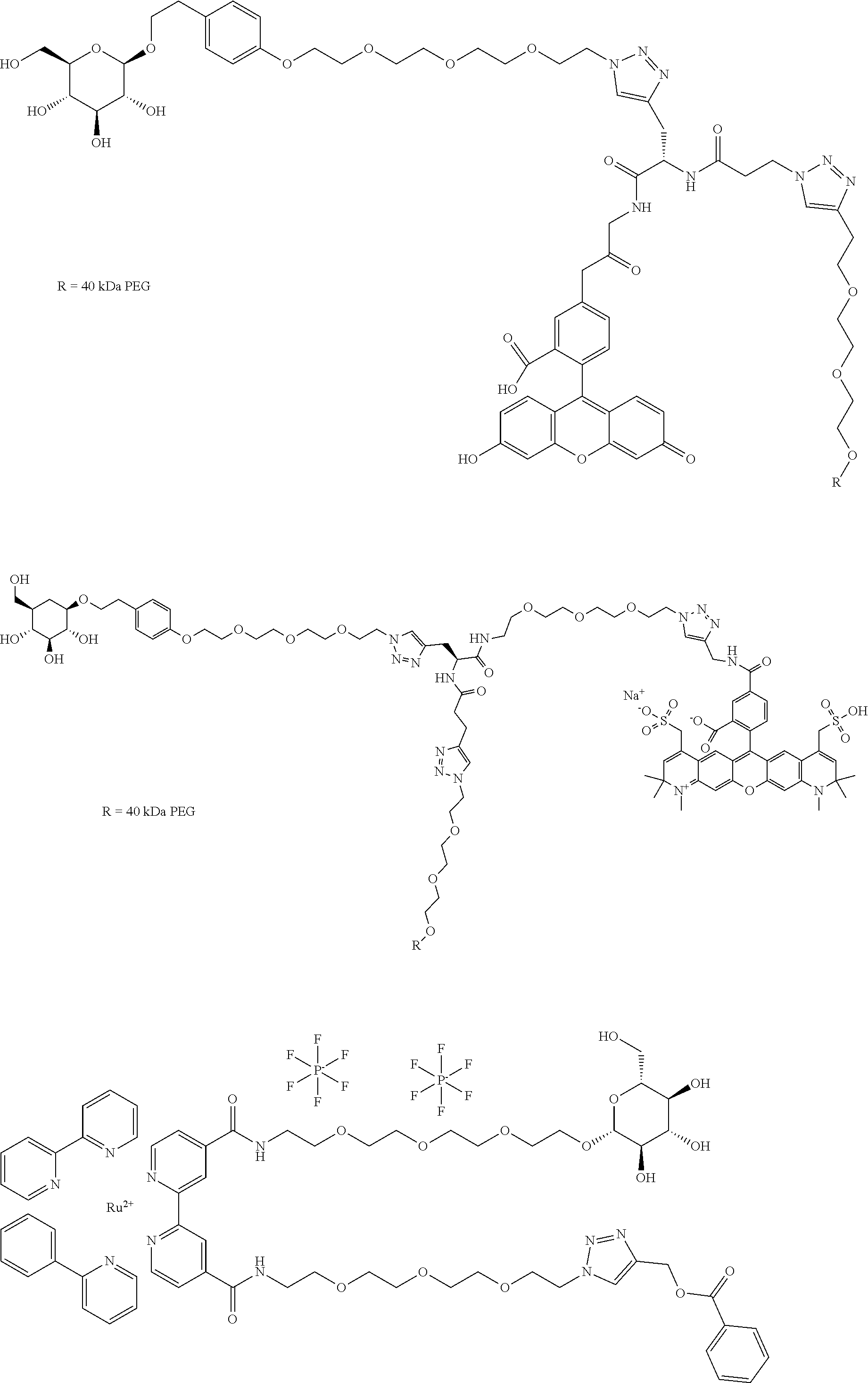

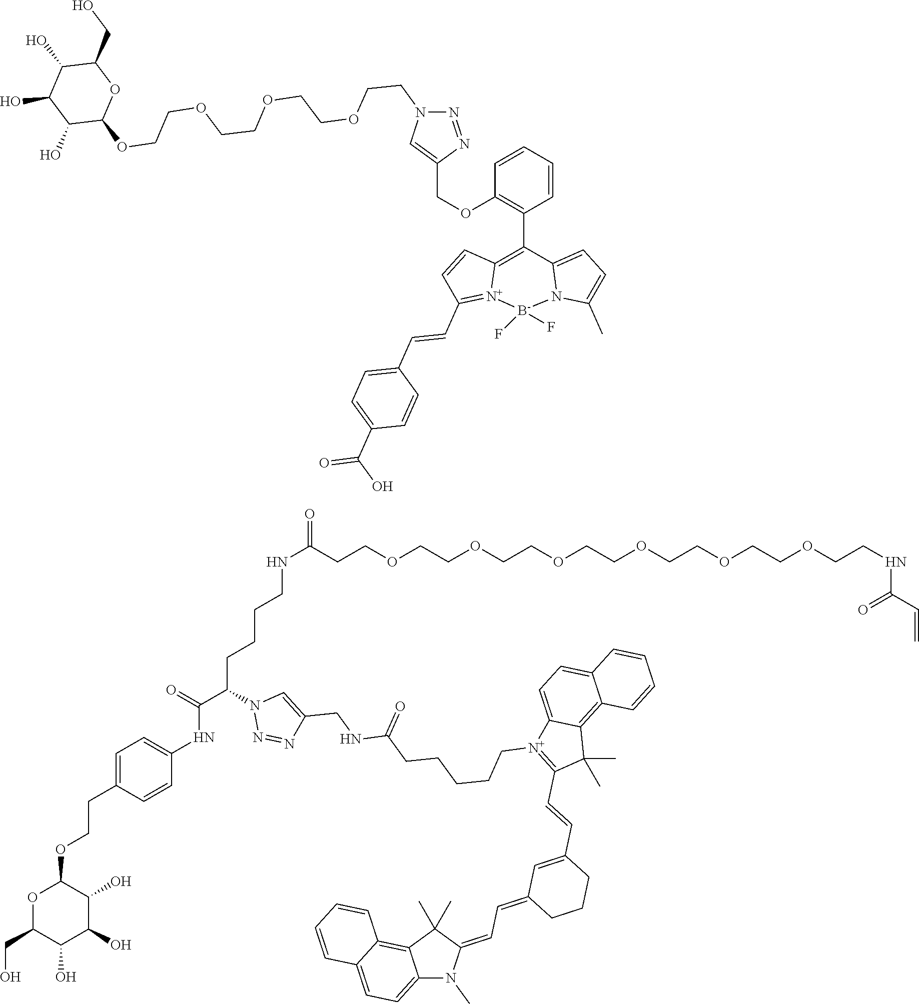 [SMILES: Cc1ccc2n1[B-](F)(F)[N+]1=C(/C=C/c3ccc(C(=O)O)cc3)C=CC1=C2c1ccccc1OCc1cn(CCOCCOCCOCCO[C@@H]2O[C@H](CO)[C@@H](O)[C@H](O)[C@H]2O)nn1 ; C=CC(=O)NCCOCCOCCOCCOCCOCCOCCC(=O)NCCCC[C@@H](C(=O)Nc1ccc(CCO[C@@H]2O[C@H](CO)[C@@H](O)[C@H](O)[C@H]2O)cc1)n1cc(CNC(=O)CCCCC[N+]2=C(/C=C/C3=CC(=C/C=C4/N(C)c5ccc6ccccc6c5C4(C)C)/CCC3)C(C)(C)c3c2ccc2ccccc32)nn1]

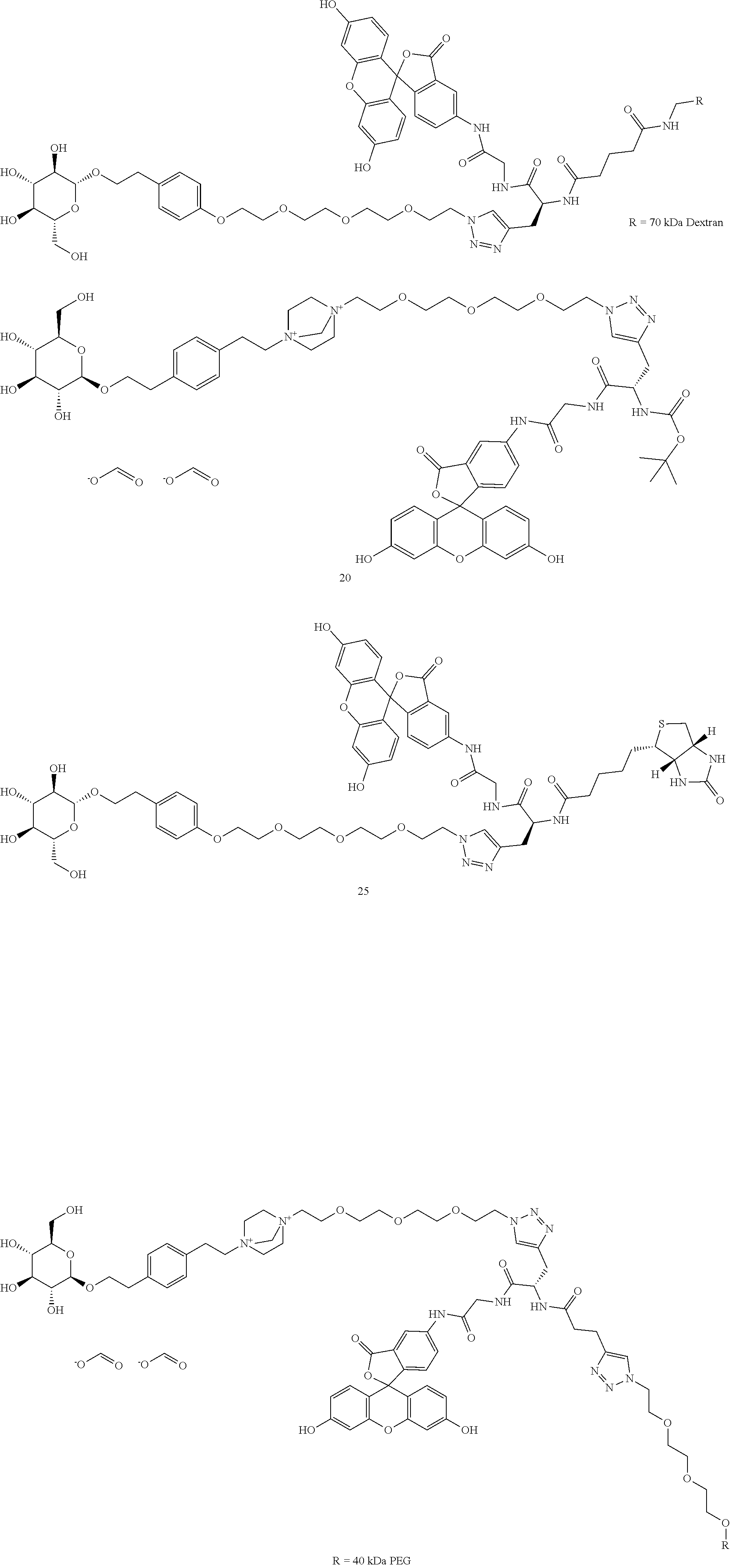

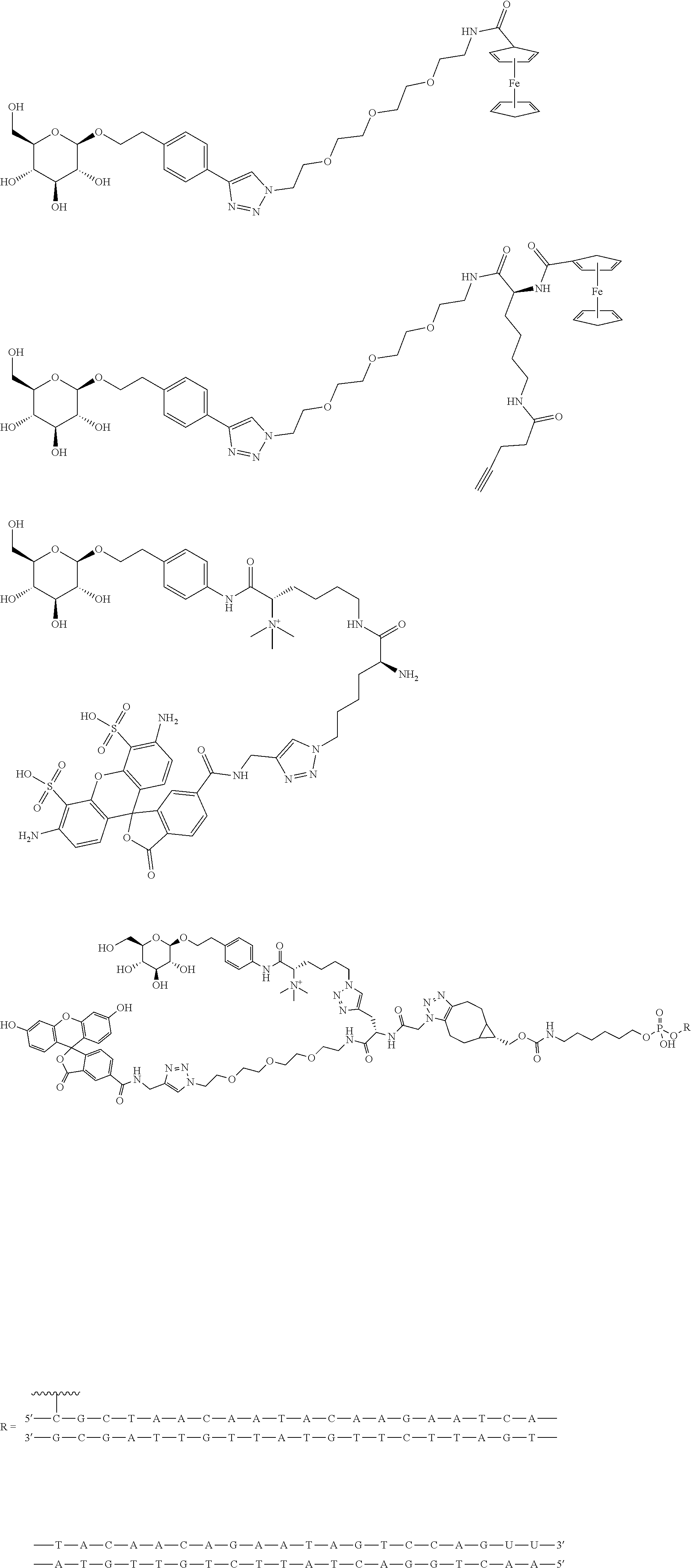

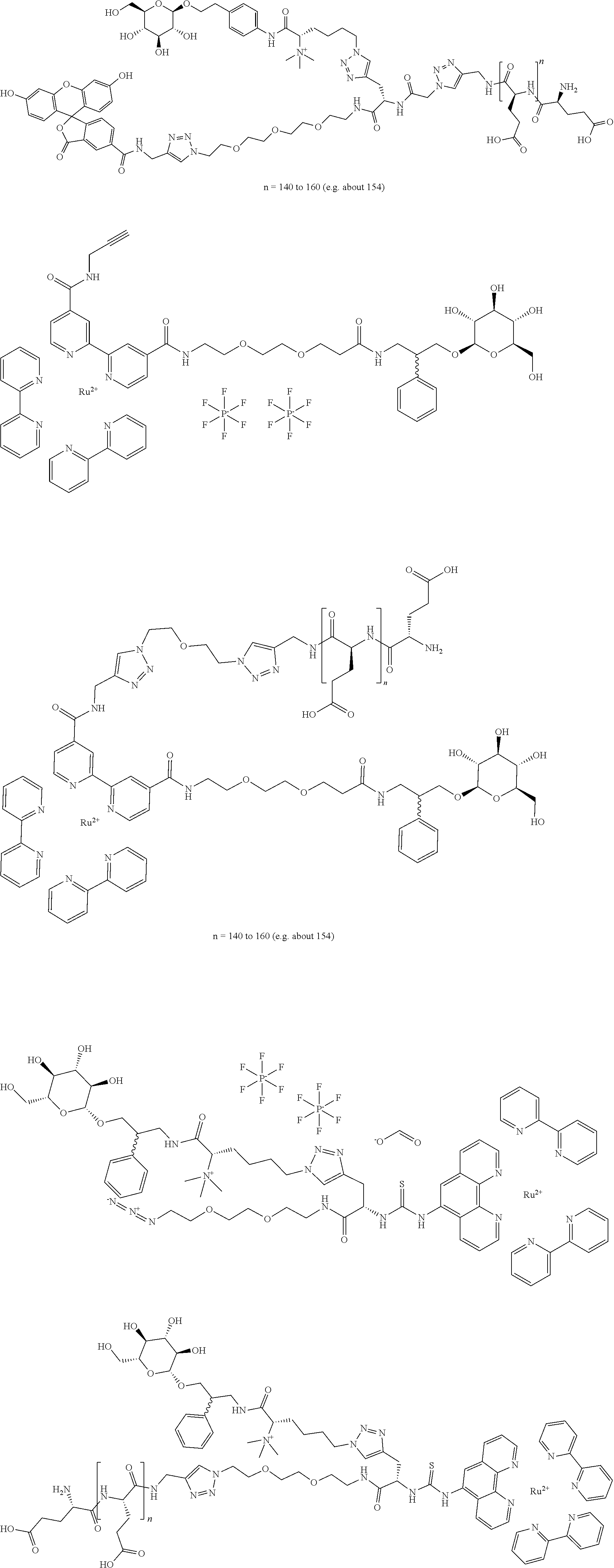

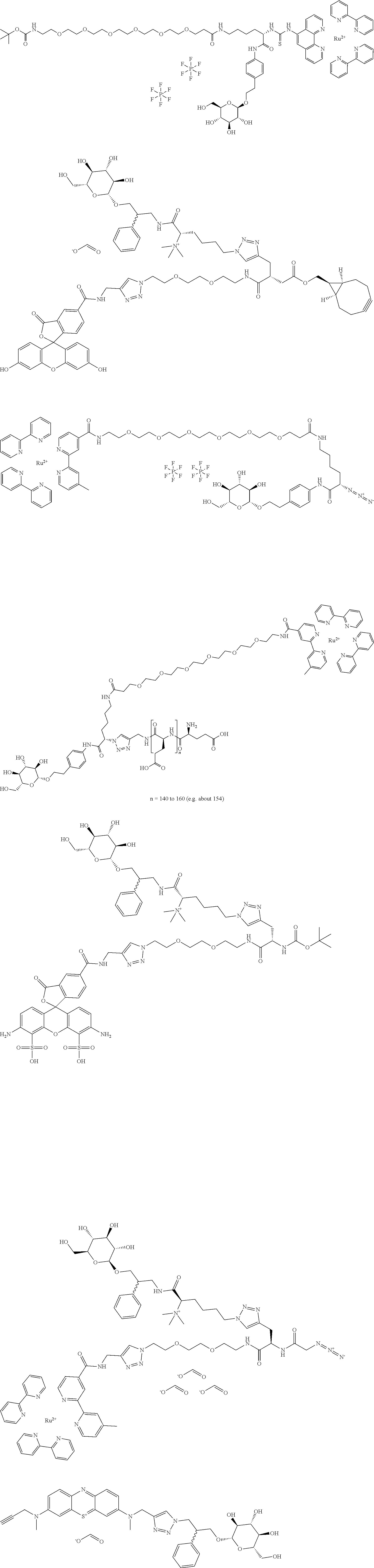

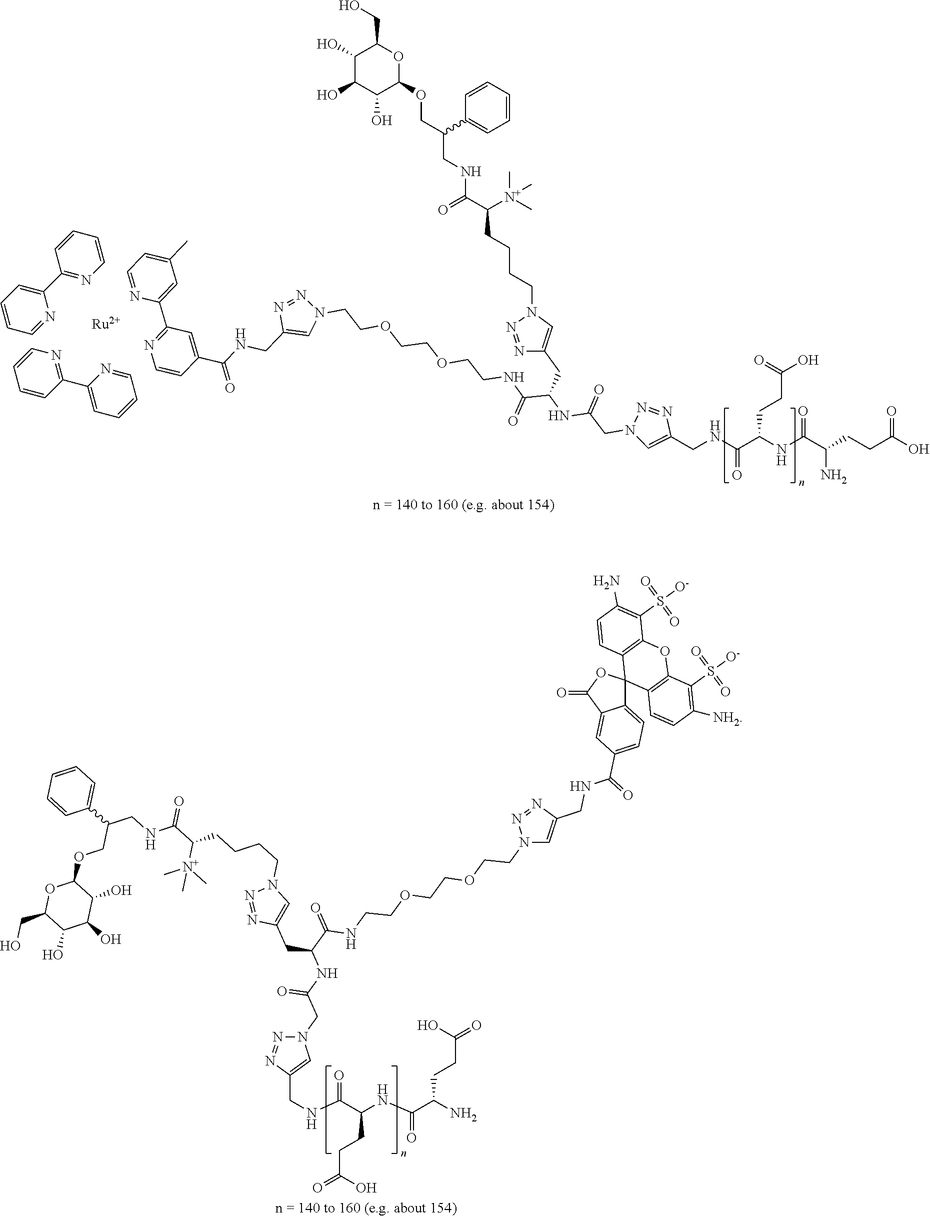

In embodiments that include a terminal group T_{L} that is capable of reacting with a semi-solid or solid support or precursors of the semi-solid or solid support (such as monomers and/or oligomers, such displaceable cavity-binding ligands may also be used as precursors to attach to displaceable cavity-binding ligand to a semi-solid or solid support and used in the third aspect of the present invention. Particularly useful terminal groups include terminal groups containing an unsaturated carbon bond, a nucleophilic group or a group susceptible of nucleophilic attack. In particular embodiments, the terminal group TL is selected from —C═CH; —CH═CH₂, bicyclo[6.1.0]nonynl, —N₃ and —NH₂.

### Semi-Solid or Solid Support

The third aspect of the present invention includes a semi-solid or solid support. The support may be a polymeric matrix and/or a gel, such as a hydrogel. The polymeric matrix and/or gel may include one polymer or a mixture of polymers. The polymeric matrix and/or a gel may include a polymer selected from polyacrylates, poly(alkylene glycols), polyacrylamides and mixtures thereof. The polymer may be a homopolymer or a copolymer. Specific examples of polymers include but are not limited to homopolymers or copolymers formed from monomers including hydroxyethylmethacrylate, ethylene glycol and acrylamide.

The polymer may be linear or branched. In certain embodiments, the polymer may be a star polymer, such as a PEG star polymer. The number of arms of the polymer may be in the range of 4 to 16. For example, the polymer matrix may include an 8-arm PEG star polymer.

In certain embodiments, the support includes a cross-linked polymer. Examples of known polymer cross-linkers include compounds with at least two reactive functional groups, such as diamines, di-esters, di-isocyanates, di-isothiocyanates, diacrylates and diacrylates. Specific examples of crosslinkers include but are not limited to triethylene glycol dimethacrylate (TEGDMA), poly(ethylene glycol) diacrylate (PEGDA) and bis-N-hydroxysuccinimidyl ester PEG.

In certain embodiments, the polymer of the semi-solid or solid support is formed from the polymerization of one or more acrylates, one or more acrylamides or mixtures thereof cross-linked with a diacrylate. In other words, the polymer may be a polyacrylate or polyacrylamide homopolymer or copolymer or a poly(acrylate acrylamide) copolymer, and the polymer is cross-linked with at least one diacrylate.

The polymeric matrix and/or gel support may include in the range of about 0.5% weight to about 10% weight of a cross-linker based on the total weight of the polymeric matrix and/or gel.

The semi-solid or solid support of the third aspect includes the saccharide-binding compound and the displaceable cavity-binding ligand independently attached to the support. Each of the saccharide-binding compound and the displaceable cavity-binding ligand are typically attached to the support by one or more linker units as described herein.

When the support includes a polymer matrix and/or gel, each of the saccharide-binding compound and the displaceable cavity-binding ligand may be independently attached to the support either before formation of the polymer matrix formation or to polymer matrix after polymerization.

The present invention of the eight to tenth aspects further provides a saccharide-binding compound described herein adapted to be immobilized on a semi-solid or solid support, a displaceable cavity binding ligand as described herein adapted to be immobilized on a semi-solid or solid support, and a method of immobilizing such saccharide-binding compound and/or displaceable cavity binding ligand onto a semi-solid or solid support.

The saccharide-binding compound described herein adapted to be immobilized on a semi-solid or solid support may be a saccharide-binding compound as defined herein wherein the compound including a terminal group capable of reacting with a semi-solid or solid support or the precursors of the semi-solid or solid support (e.g. monomers or oligomers).

The displaceable cavity binding ligand as described herein adapted to be immobilized on a semi-solid or solid support may be a displaceable cavity binding ligand as described herein having a terminal group capable of reacting with a semi-solid or solid support or the precursors of the semi-solid or solid support (e.g. monomers or oligomers).

The method of immobilizing the saccharide-binding compound and/or displaceable cavity binding ligand onto a semi-solid or solid support may include the step of reacting a saccharide-binding compound described herein adapted to be immobilized on a semi-solid or solid support and/or a displaceable cavity binding ligand as described herein adapted to be immobilized on a semi-solid or solid support with the semi-solid or solid support or the precursors of the semi-solid or solid support (e.g. monomers or oligomers).

In certain embodiments, the method includes the steps of (i) providing at least one or more organic monomers for forming a polymer semi-solid or solid support and one or both a displaceable cavity binding ligand as described herein having a terminal group capable of reacting with at least one of the organic monomers and a saccharide-binding compound as defined herein wherein the compound including a terminal group capable of reacting with at least one of the organic monomers and (ii) reacting the monomers, the displaceable cavity binding ligand and/or the saccharide-binding compound. In these embodiments, the reaction may be a polymerisation reaction to form copolymer semi-solid or solid support formed from the monomers the displaceable cavity binding ligand and/or the saccharide-binding compound.

### Systems of the First to Fourth Aspects

The systems of the first to fourth aspects typically include an aqueous environment and the saccharide-binding compound and/or detectable reporter are dissolved, dispersed, suspended or immersed in the aqueous environment. The systems of the first to fourth aspects may be used to sense the absence or presence of the target saccharide in the aqueous environment. In certain embodiments, the systems of the first to fourth aspect are used to detect the concentration of the target saccharide in the aqueous environment.

The aqueous environment may include or consist of a biological fluid or components of a biological fluid. The biological fluid may be ex vivo or in vivo. In some embodiments, the biological fluid is a bodily fluid of a subject. In particular embodiments, the bodily fluid is blood or a blood derivative such as blood plasma, blood serum and filtered blood serum. The biological fluid may be a biological sample dissolved and/or suspended in a solvent (e.g. water).

The aqueous environment may include a buffer, such as phosphate buffer. The aqueous environment may contain one or more salts, such as NaCl. The aqueous environment may be a saline solution. In more particular embodiments, the aqueous environment includes phosphate buffered saline. The aqueous environment may have a pre-determined volume. Such pre-determined volume may assist in accurate saccharide detection.

The systems of the first to fourth aspects may be housed in a detection chamber (which may also be referred to as a sensing cell). In systems of the present invention that include a fluorescent reporter moiety and/or a chromophore, the detection chamber may include at least one interrogation window for transmitting excitation radiation into the detection chamber and/or receiving a detectable signal (e.g. an emission from the fluorescent reporter moiety and/or chromophore) from the detection chamber. The interrogation window typically includes at least one section of a material that allows transmission of radiation of infrared, visible and/or ultraviolet wavelengths through the material. The detection chamber may have a single interrogation window or may have two interrogation windows (e.g. one for transmittal of radiation into the detection chamber and one for receiving a detectable signal from the detection chamber).

In some embodiments, the detection chamber has an inlet for the ingress of the target saccharide into the aqueous environment of the system. In particular embodiments, the inlet is a selectively permeable membrane. The selectively permeable membrane may form at least part of a wall of the detection chamber. The selectively permeable membrane may form a barrier between the detection chamber and a biological fluid. In this way, only part of the biological fluid may enter (and exit) the detection chamber. Typically, the selective permeable membrane allows the passage of the target saccharide across the membrane.

In particular embodiments, the selectively permeable membrane is a molecular weight cut-off membrane. The molecular weight cut-off membrane may have a molecular weight cut-off of at least about 1,000 daltons (1 kDa), at least about 2,000 daltons (2 kDa), at least about 5,000 daltons (5 kDa), at least about 10,000 daltons (10 kDa) or at least about 30,000 daltons (30 kDa).

In these embodiments, the saccharide-binding compound and detectable reporter (if separate to the saccharide-binding compound) may have a molecular weight above the molecular weight cut-off of the molecular weight cut-off membrane. In this way, the saccharide-binding compound and detectable reporter typically does not permeate across the membrane and out of the detection chamber. As described herein, the saccharide-binding compound and detectable reporter may include a membrane blocking group (MMBG or LMBG) to, for example, increase the molecular weight of these components to prevent passage of these components across the membrane and out of the detection chamber.

In the second aspect of the present invention, the detection chamber typically includes an electrode in fluid contact with the aqueous solution. In particular embodiments, the electrode is adapted to perform cyclic voltammetry.

In the third aspect of the present invention, the semi-solid or solid support may be in contact with an internal surface of the detection chamber such that the semi-solid or solid support. For example, the semi-solid or solid support may be coated on to an internal wall of the detection chamber. In this way, the semi-solid or solid support may be in fluid contact with an aqueous solution containing a target saccharide contained within the detection chamber.

The detection chamber described herein may be connected to other known components for the effective detection of the target saccharide by the systems of the first to fourth aspect.

### Devices Including One of the Systems of the First to Fourth Aspects

The present invention provides a device including one of the saccharide sensing systems of the first to fourth aspects.

In particular embodiments, the device is an implantable device including one of the saccharide sensing systems of the first to fourth aspects. As provided herein, an implantable device is a device intended to be implanted or inserted into a subject. The duration of the insertion will vary depending on the application.

The implantable device may be a probe. Such probes typically have a tip to be inserted into a subject. In some embodiments, the probe may include a detection chamber as described herein in the tip of the probe. The detection chamber in the probe will typically have an inlet adapted to be in fluid contact with a bodily fluid after insertion of the probe into the subject.

The semi-solid or solid support of the third aspect may be form an exterior surface of a probe. In this way, the semi-solid or solid support of the third aspect may be exposed to an aqueous solution containing a target saccharide by inserting probe into the aqueous solution containing the target saccharide to the extent that at least part of the semi-solid or the solid support is in fluid contact with the aqueous solution. The exterior surface may be position at the tip of a probe.

In these embodiments, the semi-solid or solid support of the third aspect may be adhered to an exterior surface of an interrogation window of the probe. The interrogation window may transmit excitation radiation to the semi-solid or solid support and/or receive a detectable signal (e.g. an emission from the fluorescent reporter moiety and/or chromophore attached to the semi-solid or solid support) from semi-solid or solid support. The interrogation window typically includes at least one section of a material that allows transmission of radiation of infrared, visible and/or ultraviolet wavelengths through the material. The probe may have a single interrogation window to which the semi-solid or solid support is adhered or may have two interrogation windows to which the semi-solid or solid support is adhered (e.g. one for transmittal of radiation into the detection chamber and one for receiving a detectable signal from the detection chamber). The internal surface of the or one of the interrogation windows may be in contact with a light guide adapted to transmit a detectable signal from the semi-solid or solid support to a detector. The light guide will typically be housed within the probe.

In alternative embodiments, the device may be a device intended to be inserted into the subject on a relatively permanent basis. For example, the device may be a device intended to be inserted in the subject for a day or more, a week or more or a month or more. Such devices are designed to detect the absence, presence and/or concentration of the target saccharide over the period of insertion. Such devices may be described as monitoring devices. In specific embodiments, the device is a continuous saccharide (e.g. glucose) monitoring device.

The monitoring device may include the detection chamber as described herein. The detection chamber in the monitoring device will typically have an inlet adapted to be in fluid contact with a bodily fluid after insertion of at least part of the device into the subject.

The subject as described herein may be a human or non-human animal subject. In particular embodiments, the subject is a human, domesticated or livestock animal. In more particular embodiments, the subject is a human.

Detecting Target Saccharide with the Systems of the First to Fourth Aspects

The present invention provides a method of detecting a target saccharide with one of the saccharide sensing systems of the first to fourth aspects.

In general, the method of detecting the target saccharide with one of the saccharide sensing systems of the first to fourth aspects includes:
(iii) contacting the saccharide sensing system with a target aqueous environment; and
(iv) measuring a detectable signal from the detectable reporter of the system.

The target aqueous environment is typically the aqueous environment, such as a biological fluid, in which the detection of the target saccharide is desired.

For the saccharide sensing systems including a fluorophore reporter moiety and/or a chromophore, the method typically includes transmitting an excitation wavelength to the saccharide sensing system. The excitation wavelength may be chosen to suit the fluorophore reporter moiety and/or chromophore. In these embodiments, the detectable signal may be an emission from the fluorophore reporter moiety and/or chromophore. One or more emission properties from the fluorophore reporter moiety and/or chromophore may be detected. The one or more emission properties include a change in intensity at one or more specific emission wavelengths, a change in emission lifetime and/or a change in the ratio of emission at two specific wavelengths.

For the saccharide sensing system of the second aspect, the method typically includes measuring a property (such as the reduction potential, oxidation potential or current) of the redox reporter moiety with the electrode. Additionally or alternatively, the method may determine an electrode property, such as the current, capacitance or voltage of the electrode or electrochemical cell. In particular embodiments, the method includes performing cyclic voltammetry on the system.

In some embodiments, the method includes a single measurement of the detectable signal from the detectable reporter of the system. The single measurement may be compared to a calibration value in order to determine the absence or presence of the target saccharide. In particular embodiments, the measurement is compared to a calibration value in order to determine the concentration of the target saccharide.

In particular embodiments, the method includes a set of two or more measurements from the detectable signal from the detectable reporter of the system over a period of time. One or more of the measurements in the set may be compared to: (i) a calibration value; (ii) one or more of the other measurements in the set of measurements; or (iii) both a calibration value and one or more of the other measurements in the set of measurements; in order to determine the absence or presence of the target saccharide. In particular embodiments, the set of two or more measurements is be compared to: (i) a calibration value; (ii) one or more of the other measurements in the set of measurements; or (iii) both a calibration value and one or more of the other measurements in the set of measurements; in order to determine the concentration of the target saccharide. The concentration of the target saccharide may advantageously be determined over the time period. In this way the concentration of the target saccharide, for example, in a subject's bodily fluid (e.g. blood) may be monitored over a period of time. Such method has use in continuous glucose monitoring.

The present invention further provides a method of detecting of abnormal target saccharide in a subject's biological fluid. The method of detection includes:
(i) detecting a target saccharide in a subject's biological fluid as described herein with one of the saccharide sensing systems of the first to fourth aspects of the present invention;
(ii) Comparing one or more detectable signal measured from the detectable reporter of the system with a standard or calibration value; and
(iii) Detecting an abnormal level of target saccharide in the subject's biological fluid based on the comparison in step (ii).

Such a method may be used to diagnosis a disease or disorder associated with an abnormal target saccharide concentration in the biological fluid. For example, the method may be used to determine diabetes from the glucose levels of a subject's blood. In particular embodiments, the method the biological fluid has been extracted from the subject before performing the method. In other words, the method is performed ex vivo.

The present invention will now be described with reference to the following non-limiting examples.

### EXAMPLES

### Materials and Methods

Commercial reagents were purchased from Sigma-Aldrich, Alfa-Aesar, Fisher Scientific, Acros Organics, Apollo Scientific Ltd, Lumiprobe GmbH, Iris Biotech GmbH, Carbosynth Ltd or Fluorochem Ltd and were used without further purification unless otherwise specified. AlexaFluor dyes are supplied as single isomers but may contain other isomers and/or contain isomeric mixtures. Modified DNA sequences were synthesised by ATDBio. All air and moisture sensitive manipulations were carried out using standard vacuum line and Schlenk techniques, or in a drybox containing a purified argon atmosphere. Solvents for air and moisture sensitive manipulations were either purchased from Sigma-Aldrich and Acros Organics or were distilled and dried over activated molecular sieves.

Normal phase medium pressure liquid chromatography (MPLC) was performed using a Biotage Isolera Four with Biotage Sfar Silica High capacity (20 μm) columns for and a suitable eluent. Reverse phase (RP) MPLC was performed using a Biotage Isolera Four with Biotage Sfar C18 D—Duo (100 Å 30 μm) columns and a suitable eluent. HPLC chromatography was performed using a Gilson Verity HPLC system with GX-271 liquid handler, 322 HPLC pump, Verity 4020 syringe pump and 171 diode array detector. For preparative runs a XBridge Prep C8 5 μm OBD (19×250 mm) column with XBridge Prep C8 5 μm (19×10 mm) guard cartridge was used with a suitable eluent. TLC was performed using aluminium backed TLC plates (Merck-Keiselgel 60 F254) and visualised using UV fluorescence and/or developed using ninhydrin, potassium permanganate, EtOH/H₂SO₄, vanillin, Pd(OAc)₂/H2O or iodine.

¹H and ¹³C NMR spectra were recorded on a JEOL Eclipse 400 MHz NMR spectrometer. All spectra were obtained at ambient temperature unless stated otherwise. All ¹H and ¹³C NMR chemical shifts are reported relative to ¹H (residual) and ¹¹C chemical shifts of the solvent as a standard. NMR spectra were processed using MestReNova software by Mestrelab Research.

Liquid chromatography mass spectrometry (LCMS) analysis was performed using either an Agilent LCMS 1260 Infinity II LC system with LC/MSD XT G6135B mass spectrometer, with Phenomenex Kinetex 5 μm Biphenyl 100 Å (150×4.6 mm) and Poroshell 120 EC-C18 2.7 μm (150×3.0 mm) columns and a suitable eluent; or an Agilent LCMS 1100 series LC system with LC/MSD SI G1956B mass spectrometer, with Modus C18 3 μm (50×2.1 mm) and Modus C8 3 μm (50×2.1 mm) columns and a suitable eluent. LCMS data were processed using MestReNova MS plugin by Mestrelab Research.

Fluorescence spectroscopy was performed on either a HORIBA Duetta or BMG Labtech CLARIOstar plus plate reader. Emission spectra were obtained using the appropriate excitation wavelength and at a temperature of either 298 or 310 K and at pH 7.4 unless otherwise stated. Data collection and analysis were performed using EzSpec software by HORIBA or CLARIOstar MARS software by BMG Labtech. Further analysis and processing were performed in Microsoft Excel.

Fluorescence lifetime measurements were performed using a HORIBA DeltaFlex-01-DD with DD-450L for excitation, PPD-850 for detection and with FiPho timing electronics. Data collection and analysis were performed using EzTime software by HORIBA. Further analysis and processing were performed in Microsoft Excel. Measurements obtained at 298 K and at pH 7.4 unless otherwise stated.

Affinity electrochemistry measurements were made using a Metrohm Autolab PGSTAT101 potentiostat with a BIDSC-FET interface and screen-printed electrodes DRP-200BT. Data collection and analysis were performed using Nova software by Metrohm Autolab. Further analysis and processing were performed in Microsoft Excel. Measurements obtained at 298 K and at pH 7.4 unless otherwise stated.

### Synthesis of Saccharide Binding Compounds

General Procedure A—HBTU amide Coupling

Starting material carboxylic acid, HBTU and HOBt·H₂O were dissolved in solvent. DIPEA was added and the reaction mixture stirred at RT for 15-30 minutes. Amine was added and the reaction stirred at RT unless otherwise specified. The reaction was monitored by LCMS until determined to be complete. The solvent was removed under vacuum and the product purified by either MPLC or HPLC if necessary.

General Procedure B—Copper click reaction

Starting material alkyne, azide and sodium L-ascorbate were dissolved in solvent. Copper (II) sulfate pentahydrate was then added and the reaction stirred at RT. The reaction was monitored by LCMS until determined to be complete. The solvent was removed under vacuum and the product purified further by either MPLC or HPLC if necessary.

### General Procedure C—Acylation

Starting material amine was dissolved in solvent. Sodium bicarbonate was added, followed by acylation reagent and the reaction stirred at RT. The reaction was monitored by LCMS until determined to be complete. If over-reaction was observed, the undesired esters were hydrolysed with aqueous sodium hydroxide prior to purification. The solvent was removed under vacuum and the product purified by either MPLC or HPLC if necessary.

General Procedure D—TFA deprotection

Starting material tBu ester or carbamate was dissolved in solvent and trifluoroacetic acid added. The reaction was stirred at RT unless otherwise stated. The reaction was monitored by LCMS until determined to be complete. The solvent was removed under a positive flow of nitrogen to give a crude product, which was re-dissolved in solvent and evaporated under a positive flow of nitrogen. This was repeated two more times to give the product which was then used without further purification.

### General Procedure E—Hydrolysis

Starting material ester suspended in solvent and sodium hydroxide added. Reaction was stirred at 40° C. and monitored by LCMS until determined to be complete. The organic solvent was removed under vacuum and the crude product precipitated with 1 M HCl. The crude product was isolated by either filtration or centrifugation and then purified further by either MPLC or HPLC if necessary.

### General Procedure F—Macrocyclisation

Starting material triamine was dissolved in pyridine and DMF and heated to 40° C. A solution of tri-isocyanate in toluene or dichloromethane was then added dropwise by syringe pump over 10 hours. The reaction was then left to stir at 40° C. and monitored by LCMS or TLC until determined to be complete. The solvent was removed under vacuum and the crude product precipitated with 1 M HCl. The crude product was isolated by either filtration or centrifugation and then purified further by either MPLC or HPLC if necessary.

General Procedure G—Isocyanate formation

Starting material amine was dissolved in anhydrous dichloromethane, followed by addition of anhydrous 2-chloropyridine. Triflic anhydride was added dropwise to the reaction mixture and the reaction stirred at RT for a further 30 minutes after addition was complete. The solvent was removed under vacuum to give a crude solid. The product was extracted with boiling petroleum ether 40/60 3 times, the extracts combined and concentrated under vacuum until a precipitate was observed. This suspension was then reheated to the boiling point until a solution was obtained, which was then cooled in a freezer at −18° C. overnight. The product crystals were then obtained by filtration, washing with cold petroleum ether 40/60 and drying under high vacuum.

### Glucose-Binding Molecule Synthesis

### Example 1: Synthesis of GBM1

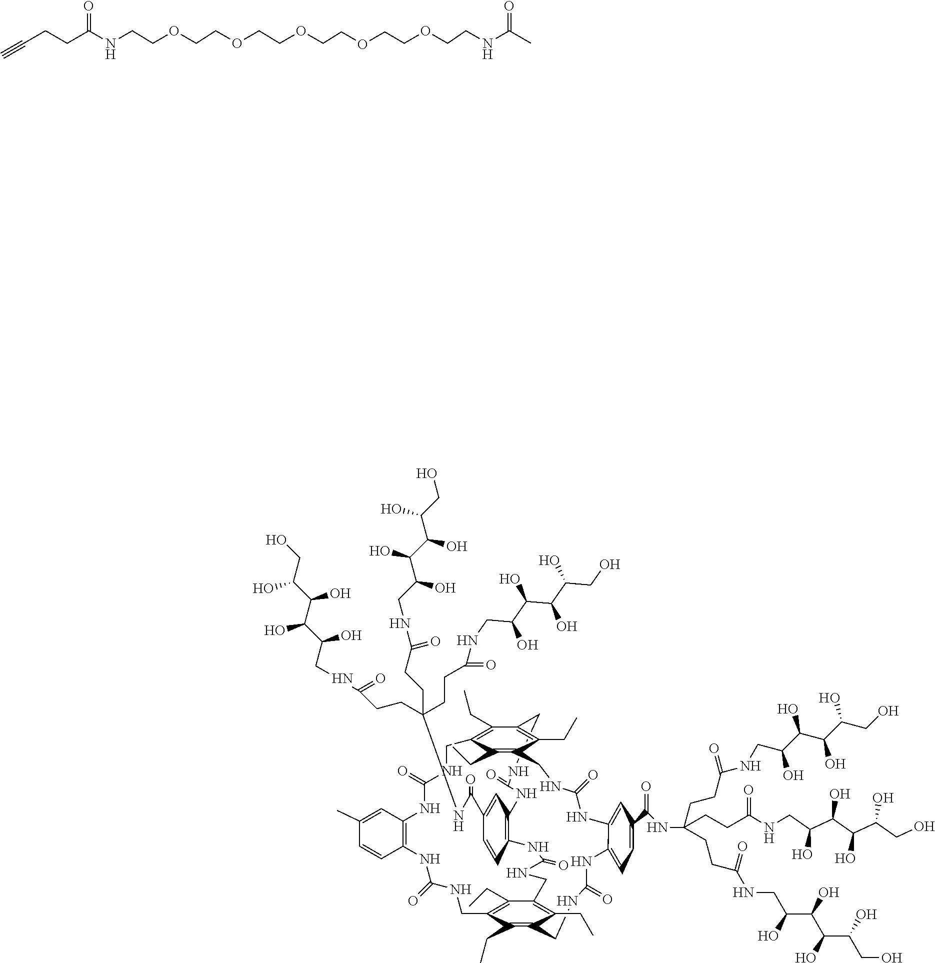

Prepared according to General Procedure A from pentynoic acid (3.0 mg, 0.030 mmol), GI5 (17 mg, 0.006 mmol), HBTU (6.9 mg, 0.018 mmol), HOBt·H₂O (2.8 mg, 0.018 mmol) and DIPEA (4.0 μL, 0.024 mmol) in DMF (0.50 mL). Purified by RP MPLC (H₂O with increasing MeCN). Product obtained as a white solid (15 mg, 0.005 mmol, 86%). MS (electrospray, +ve) [M+3H]³⁺ calculated for C₁₃₀H₂₀₇N₂₂O₅₁ requires: 964.4766, found: 964.4

### Example 2: Synthesis of GBM2

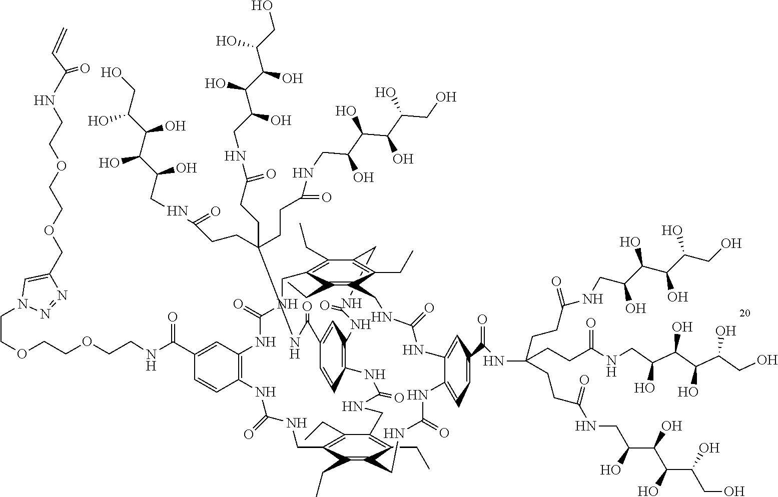

Prepared according to General Procedure C from GI7 (166 mg, 0.058 mmol), N-acryloxysuccinimide (30 mg, 0.177 mmol), NaHCO₃ (5.0 mg, 0.060 mmol) in MeCN (1.5 mL) and water (1.5 mL). Purified by RP MPLC (H₂O with increasing MeCN). Product obtained as a white solid (160 mg, 0.055 mmol, 95%).

MS (electrospray, +ve) [M+3H]³⁺ calculated for C₁₂₉H₂₀₄N₂₅O₅₀ requires: 968.1402, found: 968.1

### Example 3: Synthesis of GBM3

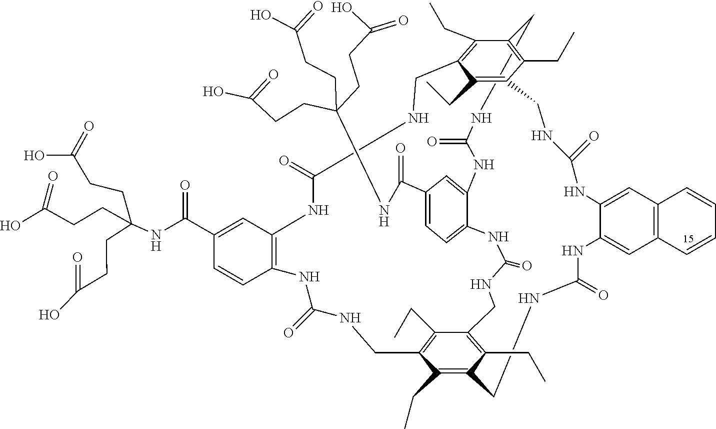

Prepared according to General Procedure A from acetic acid (1.0 μL, 0.021 mmol), GI7 (20 mg, 0.007 mmol), HBTU (8.9 mg, 0.024 mmol), HOBt·H₂O (3.6 mg, 0.024 mmol) and DIPEA (5.0 μL, 0.028 mmol) in DMF (0.30 mL). Purified by RP MPLC (H₂O with increasing MeCN). Product obtained as a white solid (19 mg, 0.007 mmol, 94%).

MS (electrospray, +ve) [M+3H]³⁺ calculated for C₁₂₃H₂₀₄N₂₅O₅₀ requires: 964.1402, found: 964.1

### Example 4: Synthesis of GBM4

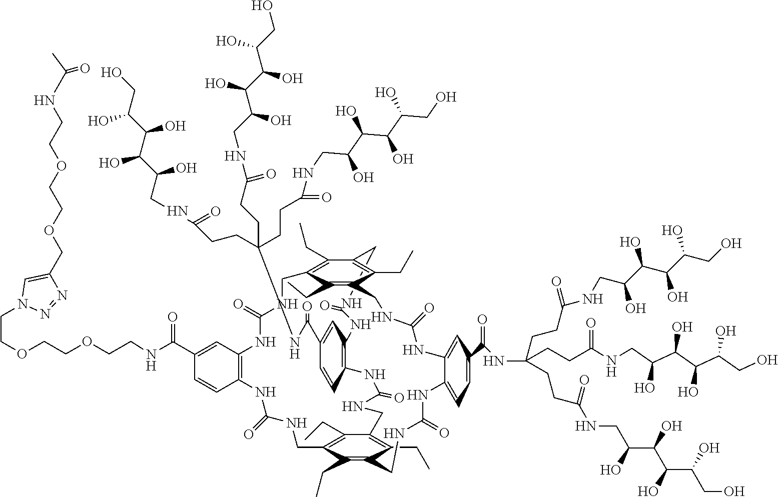

Prepared according to General Procedure D from GI16 (82 mg, 0.043 mmol) in TFA (0.8 mL) and DCM (1.6 mL). Residue resuspended in water and precipitate filtered and dried to give the product as a white solid (61 mg, 0.039 mmol, 90%).

¹H NMR (400 MHz, DMSO-d₆) δ 8.27 (s, 1H), 8.21 (s, 1H), 8.02 (d, J=2.2 Hz, 2H), 7.93 (d, J=8.6 Hz, 2H), 7.71 (dd, J=6.2, 3.4 Hz, 2H), 7.47 (dd, J=8.6, 2.2 Hz, 2H), 7.36-7.30 (m, 2H), 4.31 (s, 12H), 2.79 (s, 13H), 2.17 (t, J=8.3 Hz, 13H), 1.98 (s, 13H), 1.15 (t, J=7.0 Hz, 19H).

### Example 5: Synthesis of GBM 5

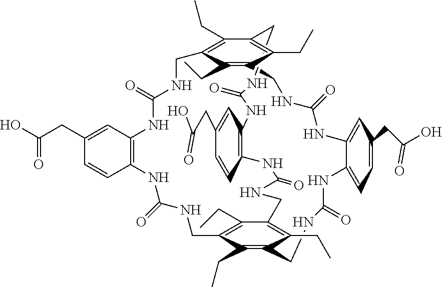

Dissolved GI13 (260 mg, 0.210 mmol) inEtOH (4 mL) and water (3 mL) and added 2 M sodium hydroxide (0.7 mL, 1.40 mmol) After 2 hours, acetic acid was added to the reaction mixture before evaporating to dryness. Crude solid redissolved using acetone/water and dry loaded onto RP silica. Purification by RP MPLC (H₂O with increasing MeCN (0.1% formic acid)). Product isolated as a white solid (90 mg, 0.078 mmol, 37%)

¹H NMR (400 MHz, DMSO-d₆+TFA) δ 7.69 (d, J=2.0 Hz, 3H), 7.64 (d, J=8.3 Hz, 3H), 7.48 (s, 3H), 7.42 (s, 3H), 6.83 (dd, J=8.4, 2.1 Hz, 3H), 6.29 (dt, J=13.9, 5.5 Hz, 7H), 4.28 (d, J=4.6 Hz, 13H), 2.80-2.64 (m, 13H), 1.12 (td, J=7.4, 4.0 Hz, 20H).

### Example 6: Synthesis of GBM 6

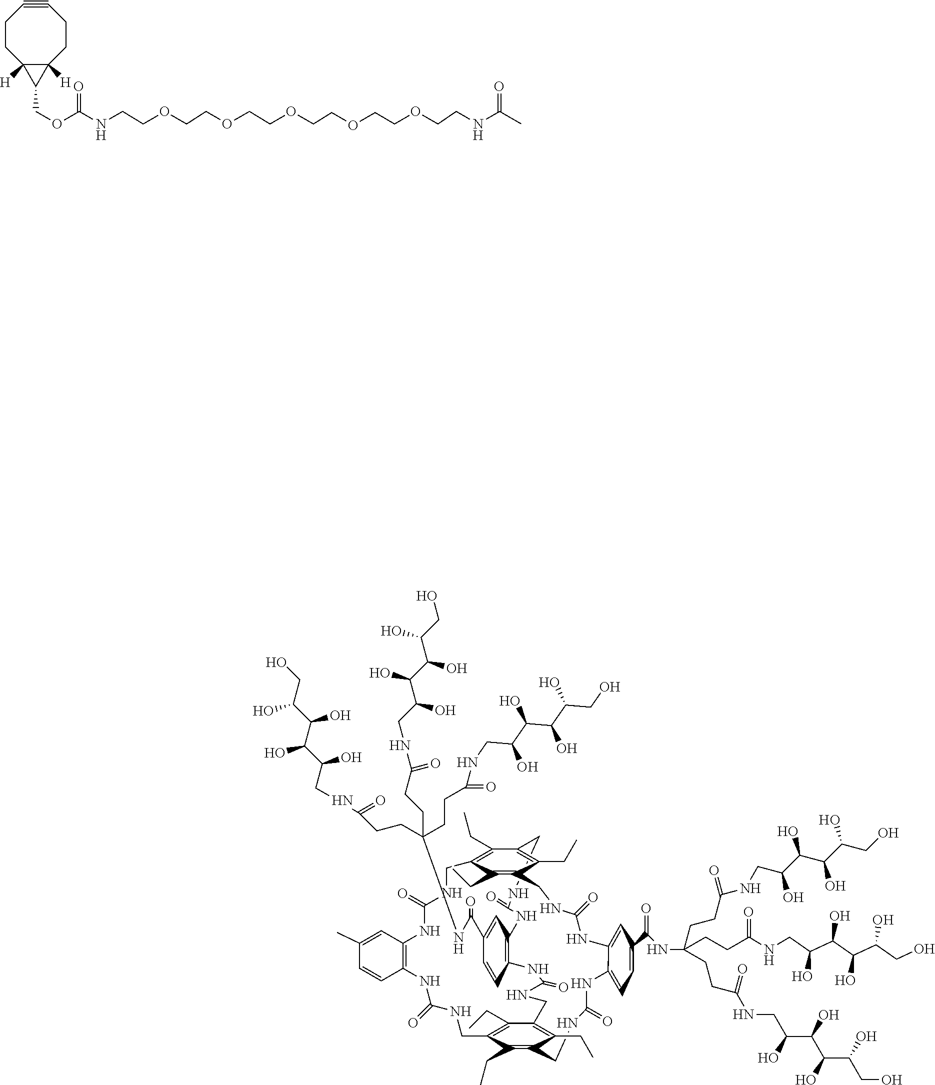

Prepared according to General Procedure C from GI5 (13 mg, 4.7 μmol), (1R,8S,9s)-bicyclo[6.1.0]non-4-yn-9-ylmethyl N-succinimidyl carbonate (4 mg, 14.2 μmol) and NaHCO₃ (1 mg, 9.5 μmol) in MeCN (0.5 mL) and water (0.5 mL). Purified by RP MPLC (H₂O with increasing MeCN (0.1% formic acid)). Product obtained as a white solid (7 mg, 2.3 μmol, 49%).

¹H NMR (400 MHz, DMSO-d₆ (with scyllo-inositol)) δ 8.40-7.69 (m, 12H), 7.49 (s, 6H), 4.78 (d, J=4.6 Hz, 6H), 4.48 (d, J=5.6 Hz, 6H), 4.44-4.19 (m, 20H), 2.24-1.82 (m, 28H), 1.14 (s, 18H), 0.97-0.95 (m, 3H).

MS (electrospray, +ve), [M+2H]²⁺ calculated for C₁₃₆H₂₁₄N₂₂O₅₂ requires: 1494.2400, found: 1494.3.

### Example 7: Synthesis of GBM7

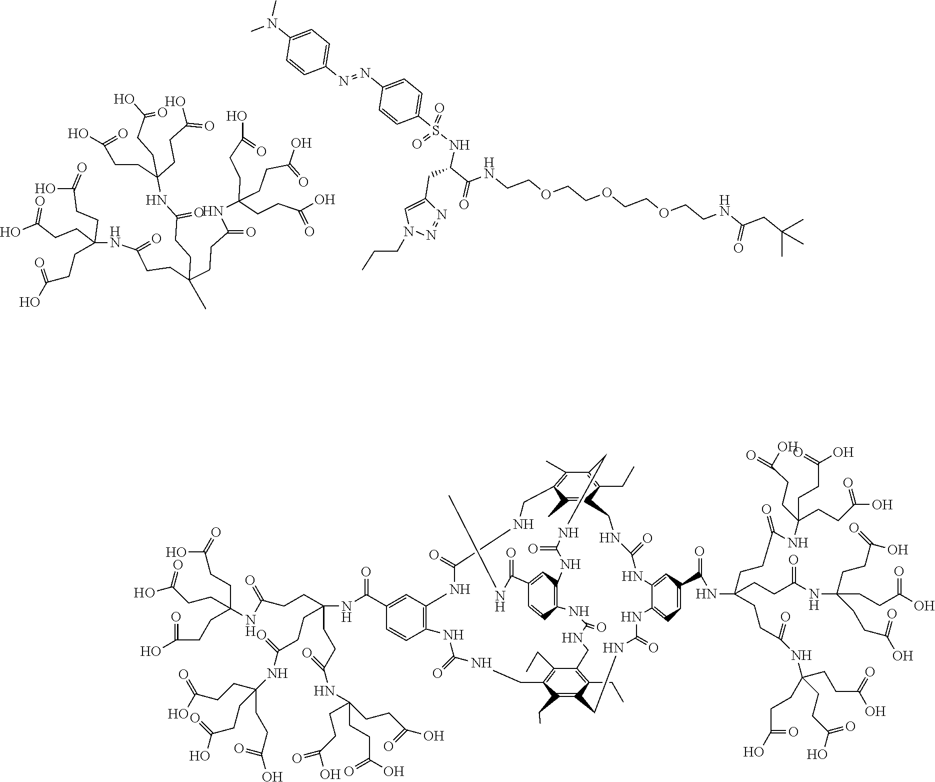

Prepared according to General Procedure B from GI48 (60 mg, 0.015 mmol), GI78 (13 mg, 0.019 mmol), CuSO₄·5H₂O (3 mg, 0.010 mmol) and sodium ascorbate (1 mg, 0.006 mmol) in THF (9 mL) and water (5 mL). Reaction mixture dry loaded on C₁₈ silica and purified by RP MPLC (H₂O with increasing MeCN (0.1% formic acid)). Product obtained as a red solid (10 mg, 0.002 mmol, 14%).

MS (electrospray, +ve), [M+3H]³⁺ calculated for C₂₀₈H₂₉₂N₃₃O₈₀S requires: 1521.9856, found: 1522.0.

### Example 8: Synthesis of GBM8

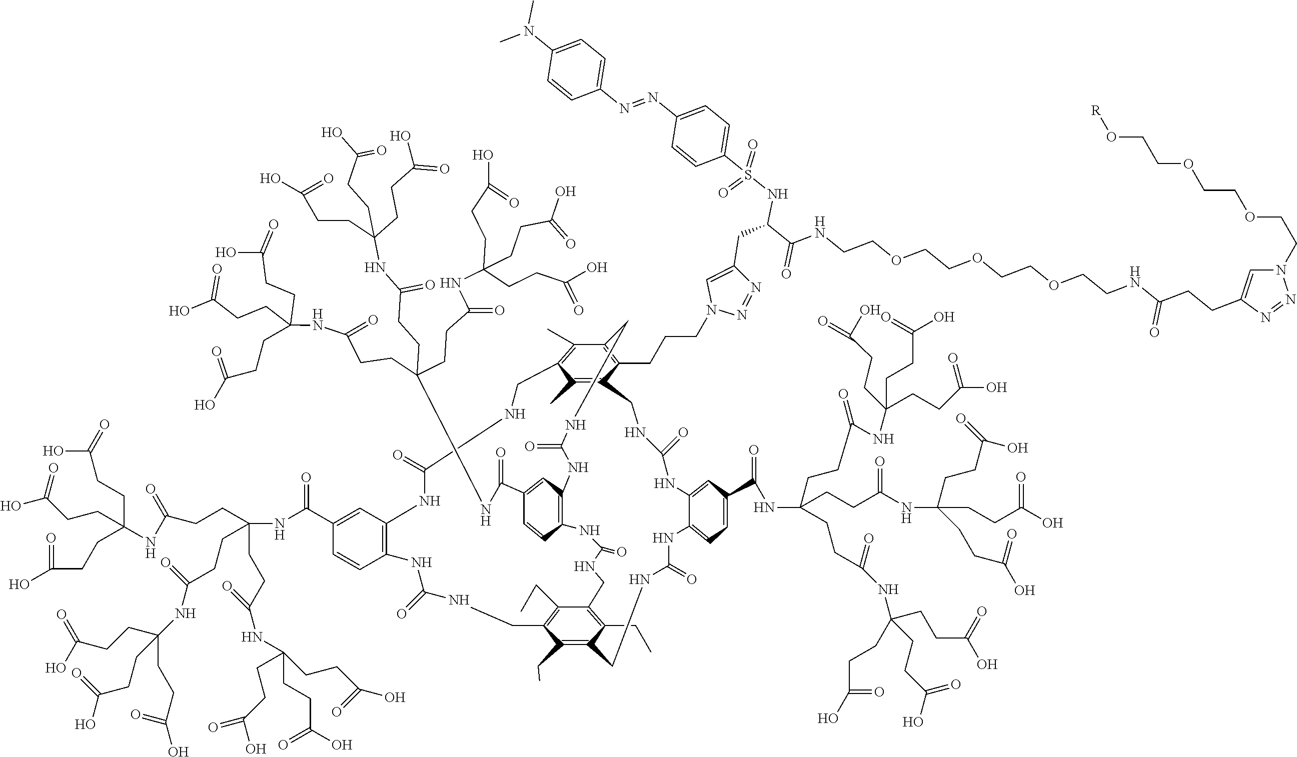

Prepared according to General Procedure B from GI50 (3 mg, 0.66 μmol), 40 kDa mPEG-N₃ (22 mg, 0.55 μmol), CuSO₄·5H₂O (1 mg, 3 μmol) and sodium ascorbate (2 mg, 11 μmol). Reaction mixture purified by dialysis (10 kDa MWCO) and resultant solution lyophilised to give the product as a brown powder (14 mg, 65%) Absₘₐₓ=270, 450 nm.

### Example 9: Synthesis of GBM9

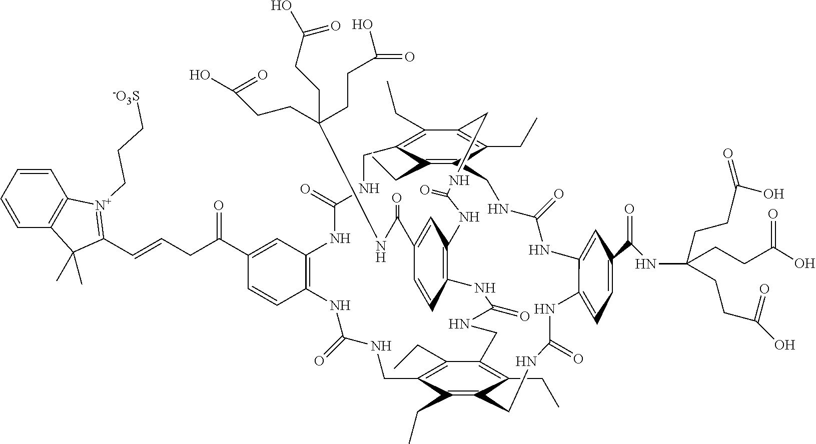

GI21 (2.5 mg, 9.0 μmol), GI20 (7 mg, 4.5 μmol) and ammonium acetate (1.0 mg, 13.5 μmol) were dissolved in DMF (110 μL) and stirred overnight. Solvent was evaporated and the residue purified by RP MPLC (H₂O with increasing MeCN (0.1% formic acid)). Product isolated as an orange solid (6 mg, 3.3 μmol, 73%).

¹H NMR (400 MHz, Deuterium Oxide (PBS), with added scyllo-inositol and DMF) δ 7.96 (s, 2H), 7.74 (d, J=9.1 Hz, 4H), 4.80 (m, 12H) 7.62 (s, 2H), 7.42 (d, J=8.6 Hz, 3H), 3.16-3.05 (2H, m), 2.77-2.54 (m, 12H), 2.46-2.36 (2H, m), 2.32-2.13 (m, 18H), 2.29-2.00 (m, 12H), 1.29-1.15 (m, 18H).

MS (electrospray, +ve) [M+H]⁺ calculated for C₉₁H₁₁₄N₁₅O₂₃S requires: 1816.7927, found: 1816.7

### Example 10: Synthesis of GBM10

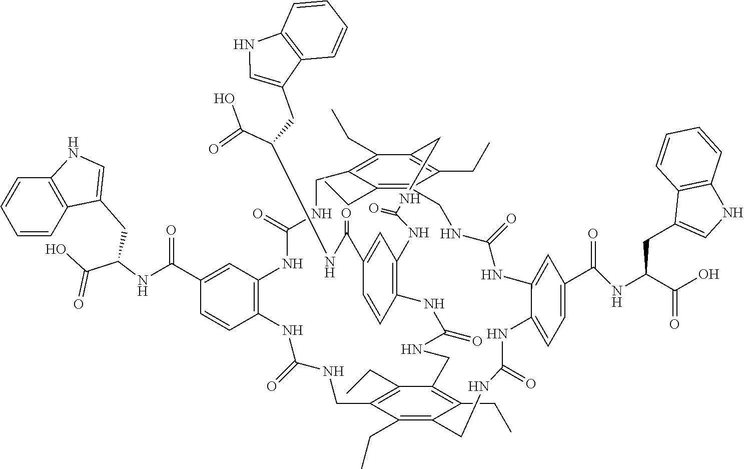 [SMILES: CCc1c2c(CC)c3c(CC)c1CNC(=O)Nc1ccc(C(=O)N[C@@H](Cc4c[nH]c5ccccc45)C(=O)O)cc1NC(=O)NCc1c(CC)c(c(CC)c(c1CC)CNC(=O)Nc1cc(C(=O)N[C@@H](Cc4c[nH]c5ccccc45)C(=O)O)ccc1NC(=O)NC3)CNC(=O)Nc1cc(C(=O)N[C@@H](Cc3c[nH]c4ccccc34)C(=O)O)ccc1NC(=O)NC2]

GI55 (417 mg, 0.244 mmol) and LiOH·H₂O (41 mg, 0.974 mmol) dissolved in THF (0.8 mL) and water (0.4 mL) and stirred for 3 hours. Reaction mixture neutralised with 1 M HCl and lyophilised. Purified by HPLC to give the product as a white solid (120 mg, 0.072 mmol, 30%).

MS (electrospray, +ve), [M+2H]²⁺ calculated for C₉₀H₉₈N₁₈O₁₅ requires: 835.3724, found: 835.5.

### Example 11: Synthesis of GBM11

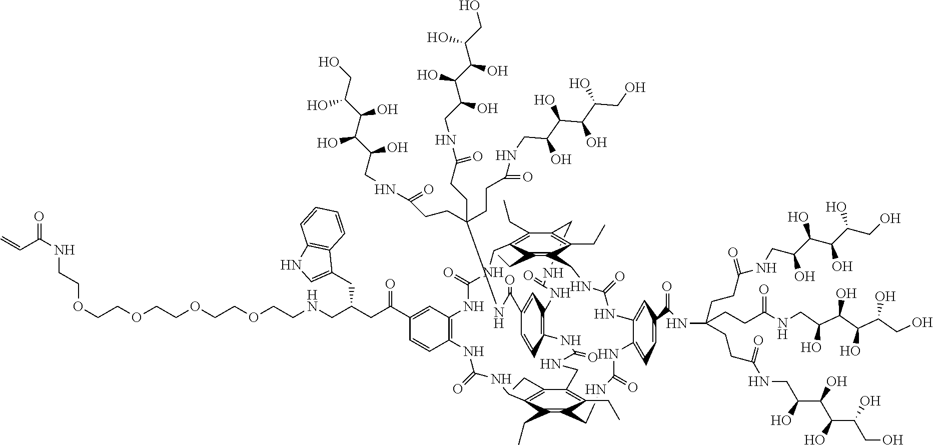

Prepared according to General Procedure C from GI59 (18 mg, 6.0 μmol), N-acryloxysuccinimide (3 mg, 18.0 μmol), NaHCO₃ (1 mg, 12 μmol) in MeCN (0.3 mL) 15 and water (0.3 mL). Purified by RP MPLC (H₂O with increasing MeCN (0.1% formic acid)) to give the product as a white solid (15 mg, 5.0 μmol, 83%).

MS (electrospray, +ve), [M+2H]²⁺ calculated for C₁₃₇H₂₁₀N₂₄O₅₁ requires: 1504.2300, found: 1504.8.

### Example 12: Synthesis of GBM12

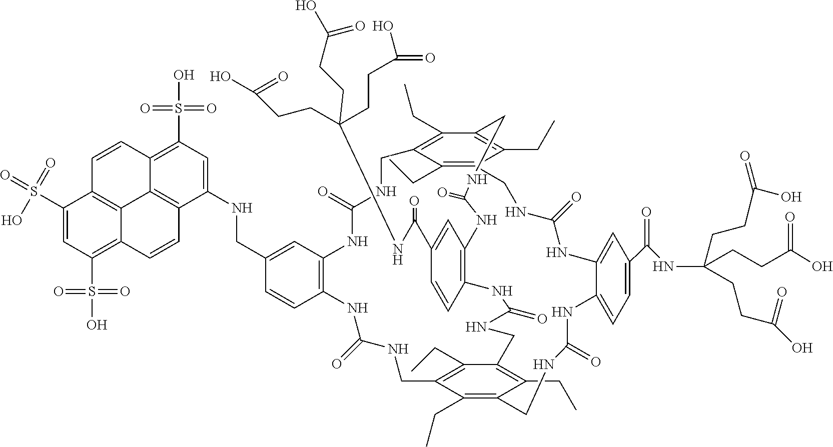 [SMILES: CCc1c2c(CC)c3c(CC)c1CNC(=O)Nc1ccc(C(=O)NC(CCC(=O)O)(CCC(=O)O)CCC(=O)O)cc1NC(=O)NCc1c(CC)c(c(CC)c(c1CC)CNC(=O)Nc1cc(C(=O)NC(CCC(=O)O)(CCC(=O)O)CCC(=O)O)ccc1NC(=O)NC3)CNC(=O)Nc1cc(CNc3cc(S(=O)(=O)O)c4ccc5c(S(=O)(=O)O)cc(S(=O)(=O)O)c6ccc3c4c65)ccc1NC(=O)NC2]

APTS (25 mg, 0.048 mmol), GI20 (15 mg, 0.010 mmol) dissolved in EtOH (1 mL), 4 Å mol. sieves and NaBH₃CN (1 mg, 0.015 mmol) added. After stirring for 6 h, NaHCO₃ (7 mg, 0.077 mmol) added, and reaction stirred for 16 hours. Reaction quenched with 0.5 M HCl and precipitate purified by RP MPLC (H₂O with increasing MeCN (0.1% formic acid)) to give the product as a yellow solid (1 mg, 0.501 μmol, 5%).

¹H NMR (400 MHz, Deuterium Oxide (PBS) with added DMF) δ 8.32 (s, 1H), 7.87-7.71 (m, 5H), 7.71-6.92 (m, 8H), 4.43-4.96 (m, 14H), 2.58 (s, 12H), 2.02 (d, J=62.1 Hz, 24H), 1.03 (s, 18H).

### Example 13: Synthesis of GBM13

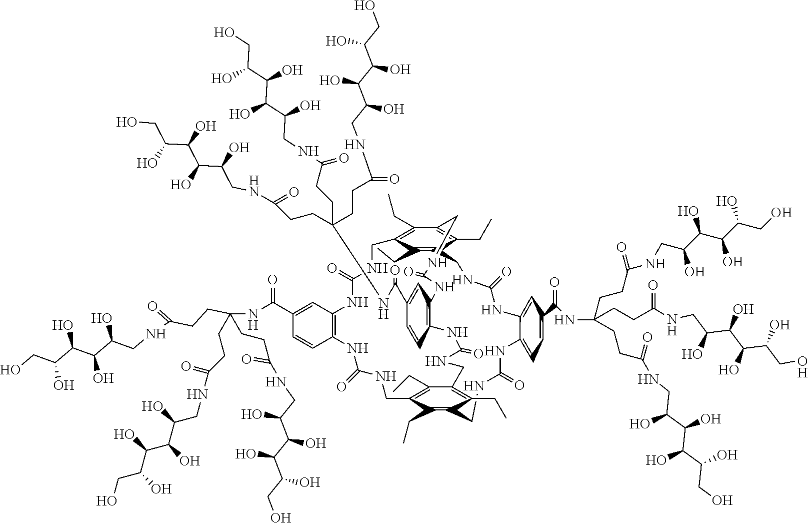

Prepared according to General Procedure A from GBM14 (50 mg, 0.028 mmol), D-glucamine (181.3 mg, 1.001 mmol), HBTU (127 mg, 0.334 mmol), HOBt·H₂O (51 mg, 0.334 mmol) and DIPEA (58 μL, 0.334 mmol) in pyridine (2.0 mL) and water (0.2 mL) heated at 45° C. Purified by RP MPLC (H₂O with increasing MeCN). Product obtained as a white solid (20 mg, 0.006 mmol, 22%).

MS (electrospray, +ve) [M+3H]³⁺ calculated for C₁₄₁H₂₃₁N₂₄O₆₃ requires: 1089.8542, found: 1089.9

### Example 14: Synthesis of GBM14

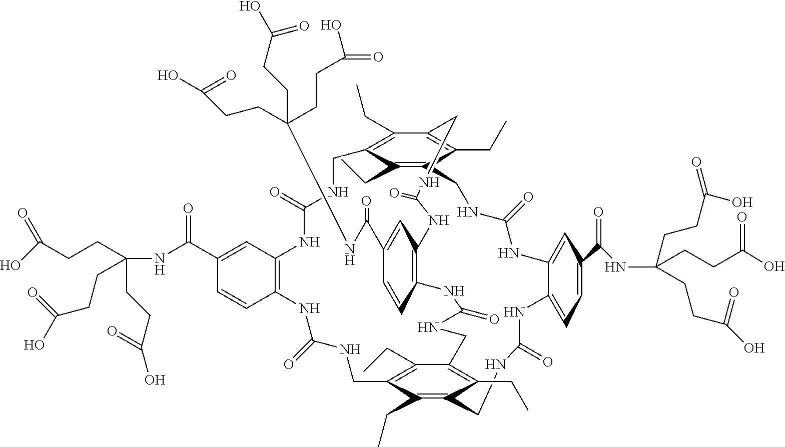

Synthesis disclosed in WO2018/167503, incorporated herein by reference.

### Example 15: Synthesis of GBM15

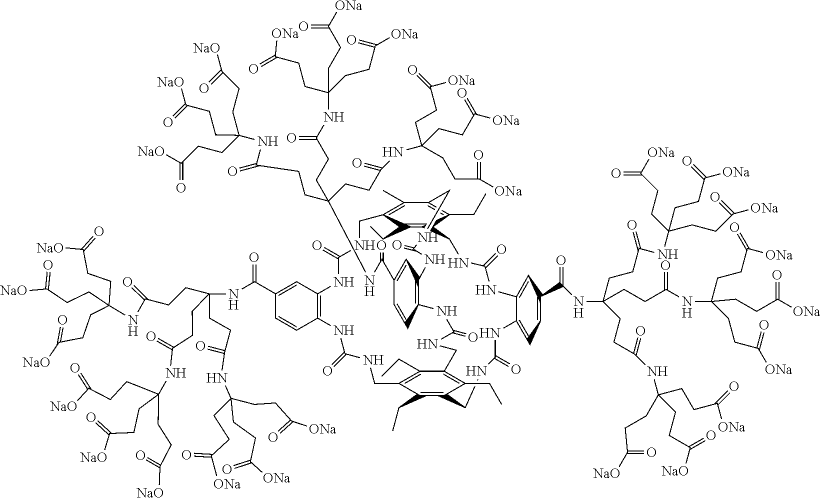

### Example 16: Synthesis of GBM16

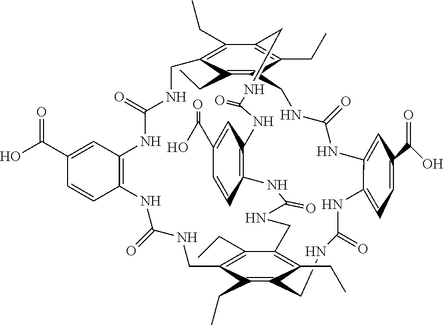

Synthesis disclosed in WO2018/167503, incorporated herein by reference.

### Example 17: Synthesis of GBM17

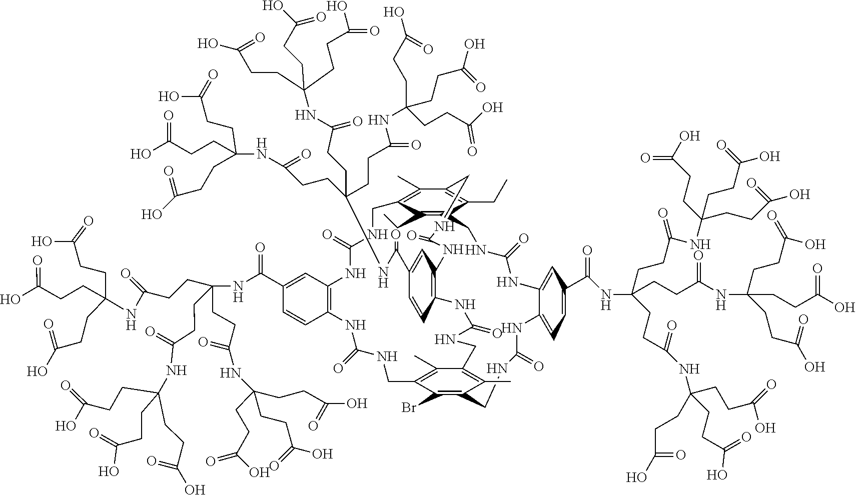

Synthesis disclosed in WO2018/167503, incorporated herein by reference.

### Example 18: Synthesis of GBM18

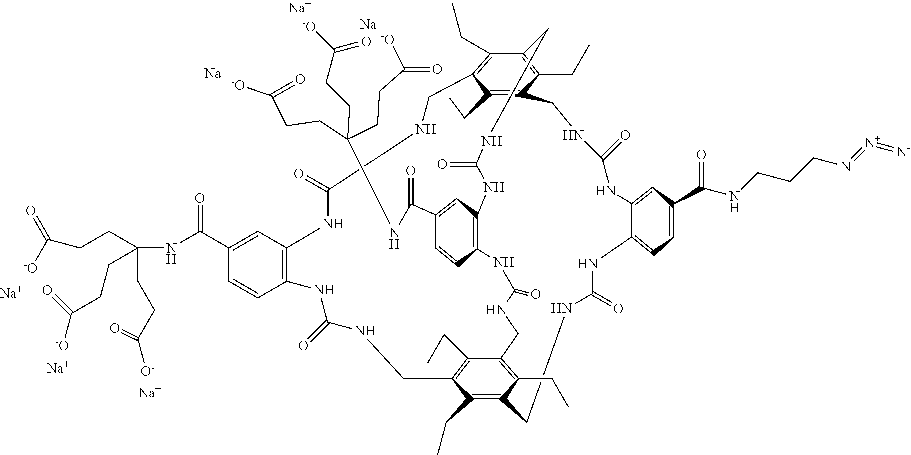

The compound was synthesised according to the synthesis of compound 21 of WO 2020/058322, the contents of which is incorporated herein.

### Example 19: Synthesis of GBM19

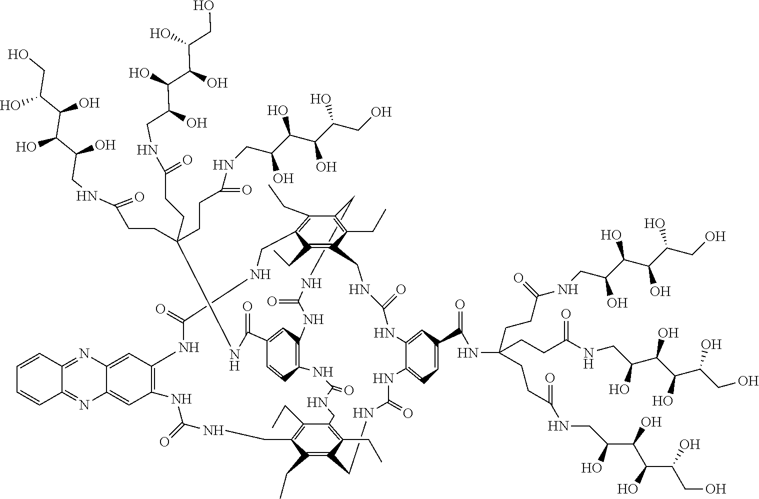

Prepared according to General Procedure A from GI1 (15 mg, 0.007 mmol), D-glucamine (181 mg, 0.166 mmol), HBTU (379 mg, 0.166 mmol), HOBt·H₂O (153 mg, 0.166 mmol) and DIPEA (10 μL, 0.055 mmol) in pyridine (1 mL) with a drop of water.

Purified by RP MPLC (H₂O with increasing MeCN (0.1% formic acid)) to give the product as an orange solid (14 mg, 0.005 mmol, 78%).

¹H NMR (400 MHz, DMSO-d₆) δ 8.55 (s, 1H), 8.51 (s, 1H), 8.19 (s, 1H), 8.15-8.09 (m, 2H), 7.93-7.78 (m, 12H), 7.50-7.41 (m, 2H), 6.62-6.38 (m, 6H), 4.46-4.17 (m, 12H), 4.14-4.07 (m, 11H), 4.00-3.90 (m, 18H), 3.64-3.55 (m, 1H), 3.56-3.50 (m, 5H), 3.49-3.43 (m, 1H), 3.42-3.33 (m, 11H), 3.30-3.19 (m, 4H), 3.08-2.94 (m, 5H), 2.86-2.55 (m, 12H), 2.18-2.01 (m, 12H), 2.01-1.89 (m, 12H), 1.22-1.03 (m, 18H).

MS (electrospray, +ve), [M+2H]²⁺ calculated for C₁₁₈H₁₇₈N₂₂O₄₄ requires: 1303.6178, found: 1303.6, 1304.1.

### Example 20: Synthesis of GBM20

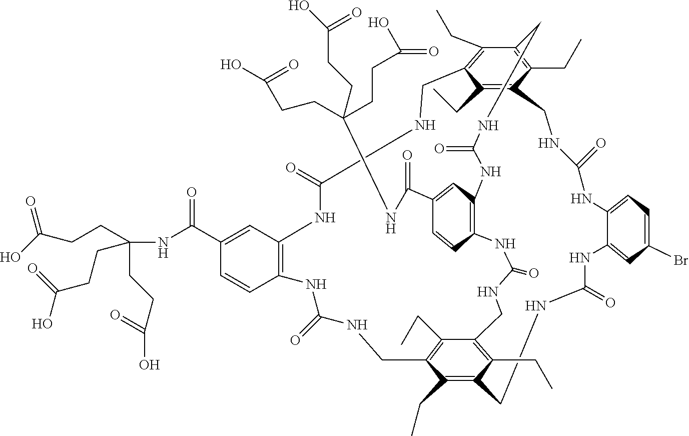

Prepared according to General Procedure D from GI66 (33 mg, 0.017 mmol) in TFA (1.7 mL) and DCM (1.7 mL). Neutralisation to pH 7 with NaOH (aq.) and freeze drying gave the product as a white solid (26 mg, 0.016 mmol, 95%).

MS (electrospray, +ve), [M+H]⁺ calculated for C₇₆H₉₆BrN₁₄O₂₀ requires: 1603.611, found: 1603.4.

### Example 21: Synthesis of GBM21

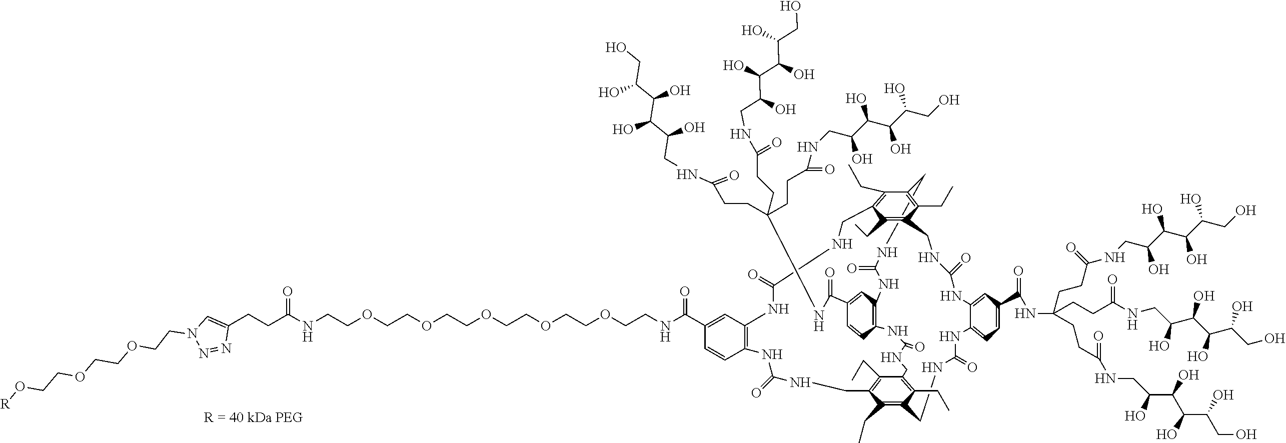

Prepared according to General Procedure B from GBM1 (3.5 mg, 1.2 μmol), 40 kDa mPEG azide (40 mg, 1 μmol), CuSO₄·5H₂O (1.2 mg, 5 μmol) and sodium ascorbate (4 mg, 20 μmol) in water (1 mL). Purified by dialysis (10k MWCO) in water (1.5 L) for 24 hours and freeze dried to give a white solid (40 mg, 1 μmol, 90%).

Absₘₐₓ=270 nm.

### Example 22: Synthesis of GBM22

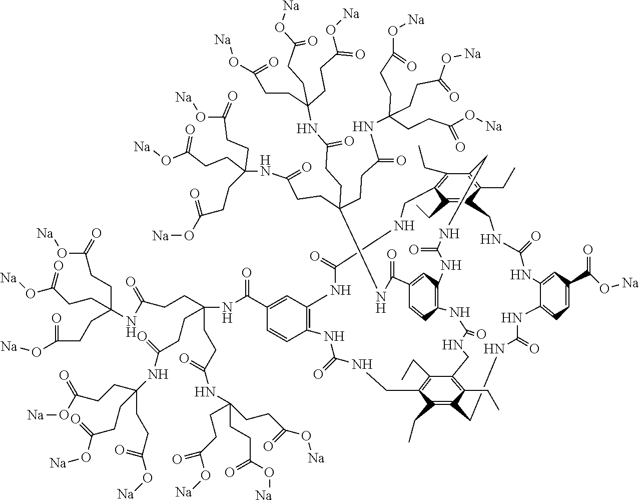

GI70 (48.0 mg, 0.012 mmol) dissolved in 0CM (1.0 mL). Added TFA (0.5 mL) dropwise. Stirred under air at RT for 18 hours. Blew down the volatiles under stream of N₂ to dryness. Suspended in H₂O and added NaOH until pH 7. Concentrated under reduced pressure to give product as a white solid (10 mg, 0.003 mmol, 25%).

¹H NMR (400 MHz, Deuterium Oxide with 2 mM DMF) 7.77-7.49 (m, 2H), 7.43 (d, J=8.0 Hz, 1H), 4.31 (s, 5H), 2.88 (s, 3H), 2.72 (d, J=0.9 Hz, 3H), 2.58 (s, 4H), 2.17 (s, 4H), 1.02 (s, 6H).

### Example 23: Synthesis of GBM23

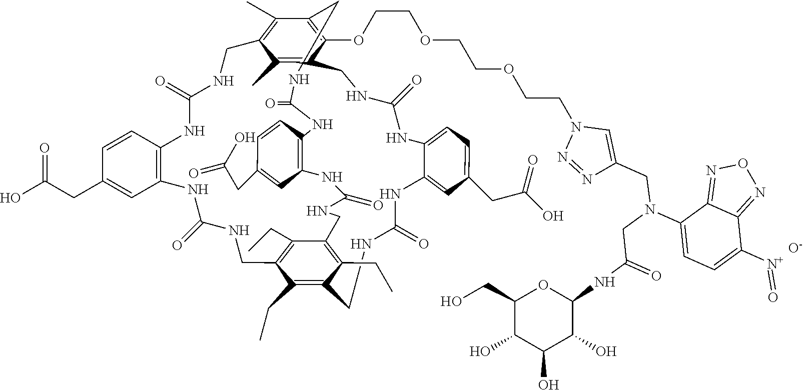

GI41 (15 mg, 0.012 mmol), GI73 (8 mg, 0.018 mmol) and sodium ascorbate (5 mg, 0.024 mmol) were dissolved in degassed water (2 mL) and THF (0.2 mL). CuSO₄·5H₂O (1 mg, 0.004 mmol) was added, and the reaction was stirred for 3 d. The reaction mixture was diluted with acetone and dry-loaded on ˜3 g C₁₈ silica. Purification using RP MPLC (H₂O with increasing MeCN (0.1% formic acid)). Product obtained as an orange solid (14 mg, 0.008 mmol, 69%).

¹H NMR (400 MHz, Deuterium Oxide (PBS)) δ 8.42 (s, 3H), 7.53 (s, 6H), 6.96 (d, J=8.5 Hz, 3H), 4.24-4.03 (m, 10H), 3.80 (s, 7H), 3.65 (s, 7H), 3.46 (d, J=14.6 Hz, 18H), 2.65 (s, 12H), 2.44-1.99 (m, 20H), 1.20 (s, 18H).

MS (electrospray, +ve) [M+2H]²⁺ calculated for C₇₉H₉₆N₂₀O₂₄ requires: 854.3448, found: 854.4.

### Example 24: Synthesis of GBM24

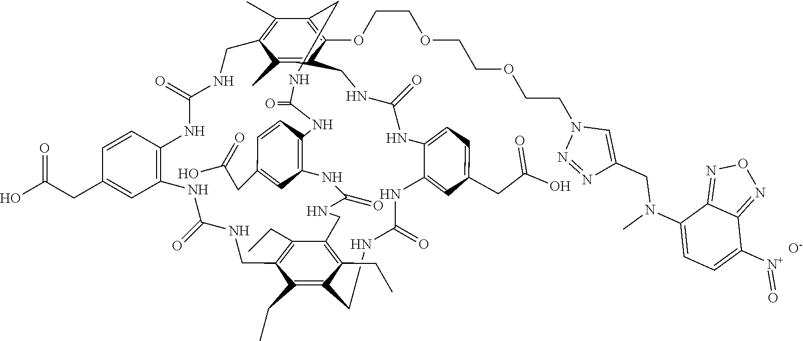

GI41 (11 mg, 0.009 mmol), GI77 (3.1 mg, 0.013 mmol) and sodium ascorbate (3.4 mg, 0.003 mmol) were dissolved in in degassed water (0.8 mL) and THF (0.2 mL). CuSO₄·5H₂O (1 mg, 0.004 mmol) was added, and the reaction was stirred for 16 hours. More GI77 (10 mg, 0.043 mmol), sodium ascorbate (3.4 mg, 0.003 mmol) and CuSO₄·5H₂O (1 mg, 0.004 mmol) were added and the reaction was stirred for a further 24 hours. Solvents were evaporated, and the residue purified by RP MPLC (H₂O with increasing MeCN (0.1% formic acid)). Product obtained as an orange solid (3 mg, 0.002 mmol, 23%).

MS (electrospray, +ve) observes dearylation, [M−Ar+H]⁺ calculated for C₆₆H₈₃N₁₆O₁₅ requires: 1339.6219, found: 1339.5.

### Example 25: Synthesis of GBM25

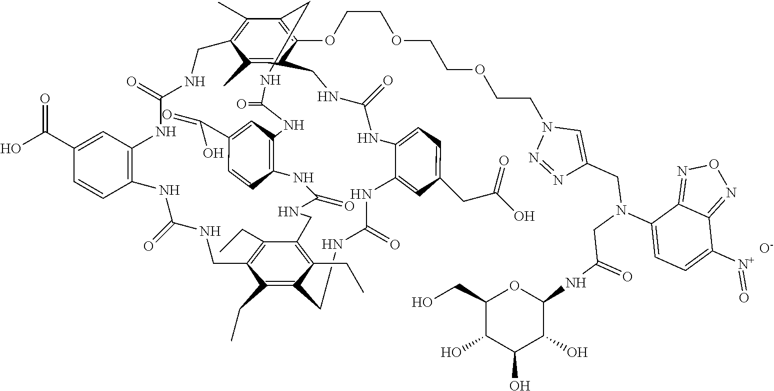

GI43 (5 mg, 0.004 mmol), GI73 (2.8 mg, 0.006 mmol) and sodium ascorbate (1.6 mg, 0.008 mmol) were dissolved in in degassed water (0.8 mL) and THF (0.2 mL). CuSO₄·5H₂O (1 mg, 0.004 mmol) was added, and the reaction was stirred for 2 hours. Solvents were evaporated, and the residue purified by RP MPLC (H₂O with increasing MeCN (0.1% formic acid)). Product obtained as an orange solid (4 mg, 0.002 mmol, 59%).

MS (electrospray, +ve) [M+H]⁺ calculated for C₇₅H₈₉N₂₀O₂₄ requires: 1665.6635, found: 1665.5.

### Example 26: Synthesis of GBM26

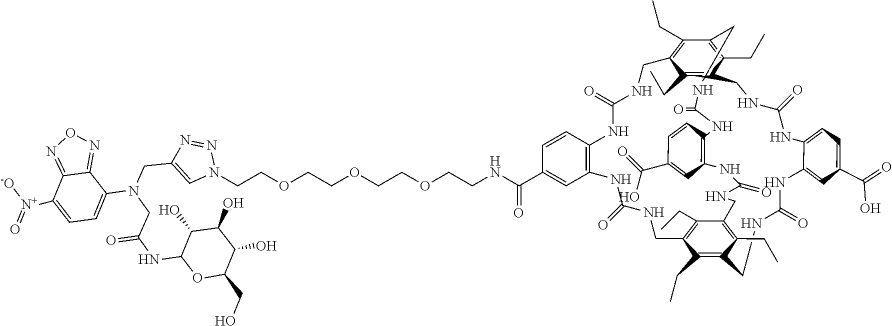

GI45 (11 mg, 0.008 mmol), GI73 (4.4 mg, 0.010 mmol) and sodium ascorbate (3.3 mg, 0.003 mmol) were dissolved in in degassed water (0.7 mL) and THF (0.5 mL). CuSO₄·5H₂O (1 mg, 0.004 mmol) was added, and the reaction was stirred for 2 hours. Solvents were evaporated, and the residue purified by RP MPLC (H₂O with increasing MeCN (0.1% formic acid)). Product obtained as an orange solid (4 mg, 0.002 mmol, 27%).

MS (electrospray, +ve), [M+H]⁺ calculated for C₃₂H₁₀₂N₂₁O₂₃ requires: 1748.7452, found: 1748.7.

### Example 27: Synthesis of GBM27

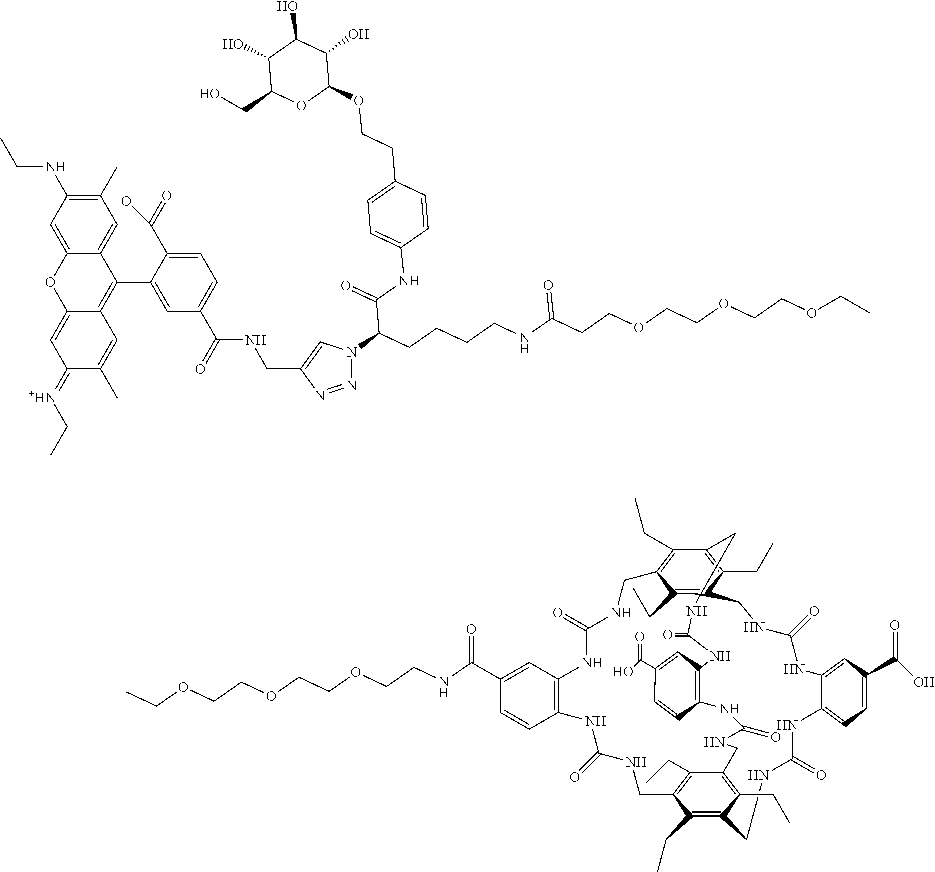

Prepared according to General Procedure E. GI46 (10 mg, 0.004 mmol) was dissolved in EtOH (1 mL) and water (1 mL), NaOH (35 mg, 0.875 mmol) was added and the reaction stirred and heated at 40° C. for 8 hours. The reaction mixture was neutralised with 1 M HCl and the EtOH evaporated, and the remnants purified by RP MPLC (H₂O with increasing MeCN (0.1% formic acid)). Product obtained as a red solid (2 mg, 0.001 mmol, 20%).

MS (electrospray, +ve), [M+2H]²⁺ calculated for C₁₂₂H₁₅₅N₂₁O₂₉ requires: 1189.5661, found: 1189.6.

### Example 28: Synthesis of GBM28

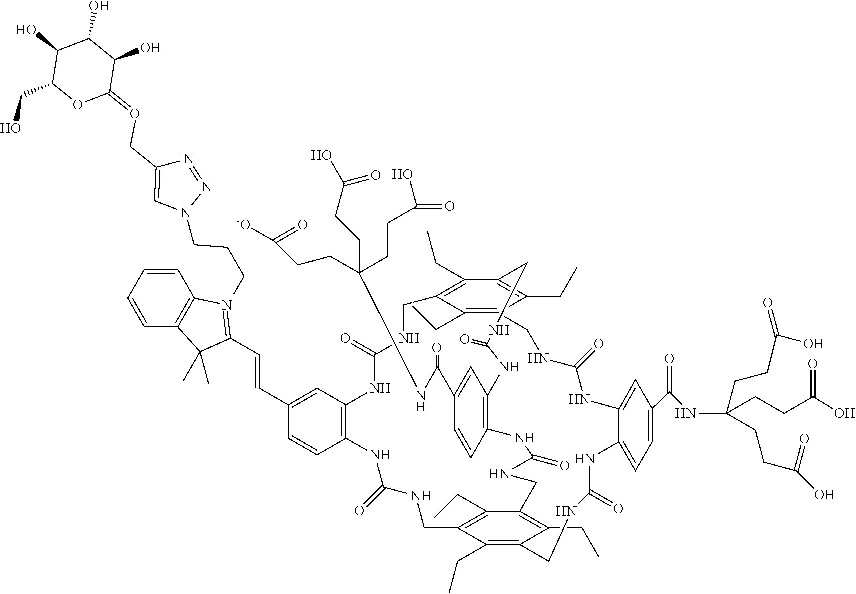

GI23 (8 mg, 4.5 μmol), sodium ascorbate (4 mg, 18.0 μmol), sodium carbonate (2 mg, 22.5 μmol) and propargyl alpha-D-glucopyranoside (2 mg, 10.8 μmol) were dissolved in water (0.6 mL) and the solution sparged. A sparged solution of CuSO₄·5H₂O (1 mg, 5 μmol) in water (0.1 mL) was added and the reaction left to stir for 25 hours. Reaction mixture loaded directly onto RP MPLC (H₂O with increasing MeCN (0.1% formic acid)). Product isolated as an orange solid (2 mg, 1.0 μmol, 22%).

¹H NMR (400 MHz, Deuterium Oxide (PBS) with added scyllo-inositol and DMF) δ 8.19-6.82 (m, 16H), 4.05 (s, 12H), 3.02 (s, 2H), 2.53 (s, 12H), 2.07 (s, 18H), 1.91 (s, 18H), 1.08 (s, 18H).

MS (electrospray, +ve) [M+2H]²⁺ calculated for C₁₀₀H₁₂₃N₁₃O₂₆ requires: 998.96835, found: 998.6.

### Example 29: Synthesis of GBM29

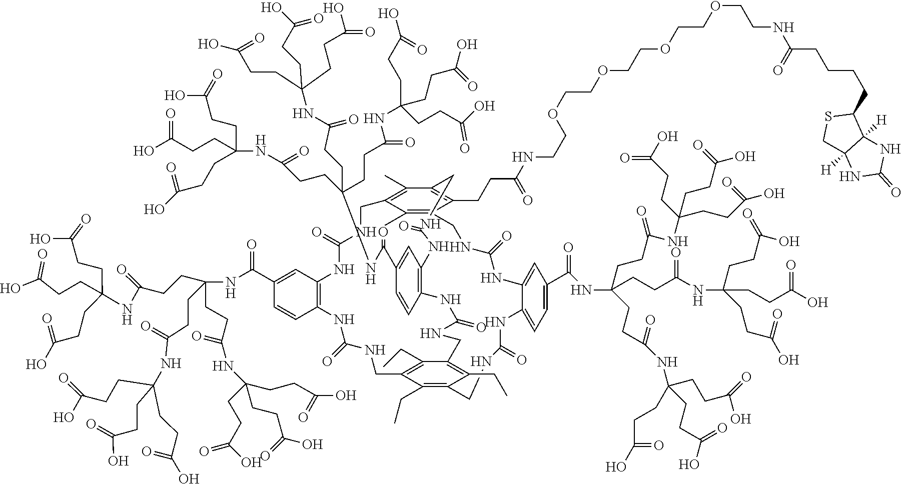

Prepared according to General Procedure 0 from GI83 (8 mg, 1.37 μmol) in TFA (0.4 mL) and DCM (0.8 mL). Purified by RP MPLC (H₂O with increasing MeCN (0.1% formic acid)) to give the product as a white solid (4 mg, 0.93 μmol, 68%).

MS (electrospray, +ve), [M+3H]³⁺ calculated for C₁₉₆H₂₃₁N₂₃O₇₉S requires: 1441.6201, found: 1441.5.

### Example 30: Synthesis of GBM30

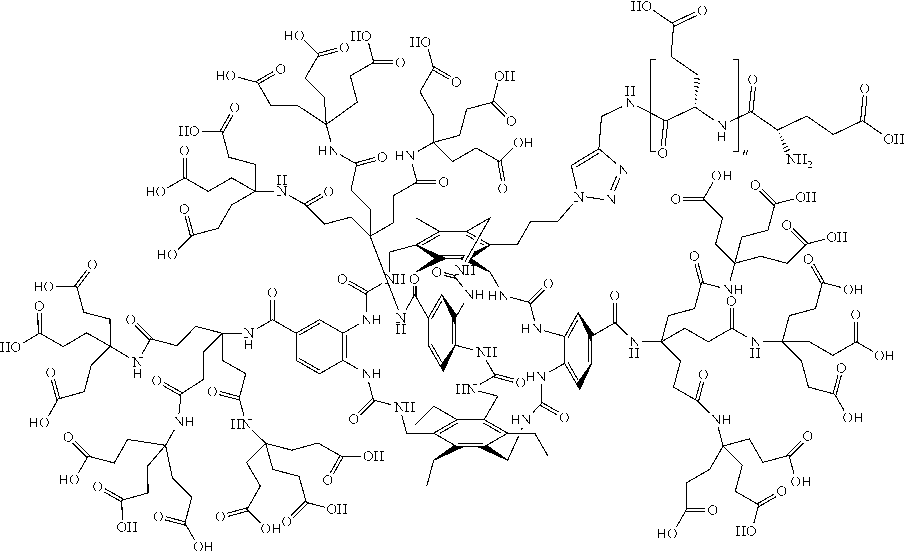

Prepared according to General Procedure B from GI46 (27 mg, 0.007 mmol), Alkyne-Poly(L-Glu) (154) (100 mg, 0.005 mmol), sodium ascorbate (20 mg, 0.099 mmol), CuSO₄·5H₂O (6 mg, 0.025 mmol) and NaHCO₃ (11 mg, 0.134 mmol) in water (13 mL).

Product obtained by dialysis (6k MWCO) of reaction mixture, followed by lyophilisation to give an off-white powder (70 mg, 0.003 mmol, 59%).

Absₘₐₓ=270 nm

### Example 31: Synthesis of GBM31

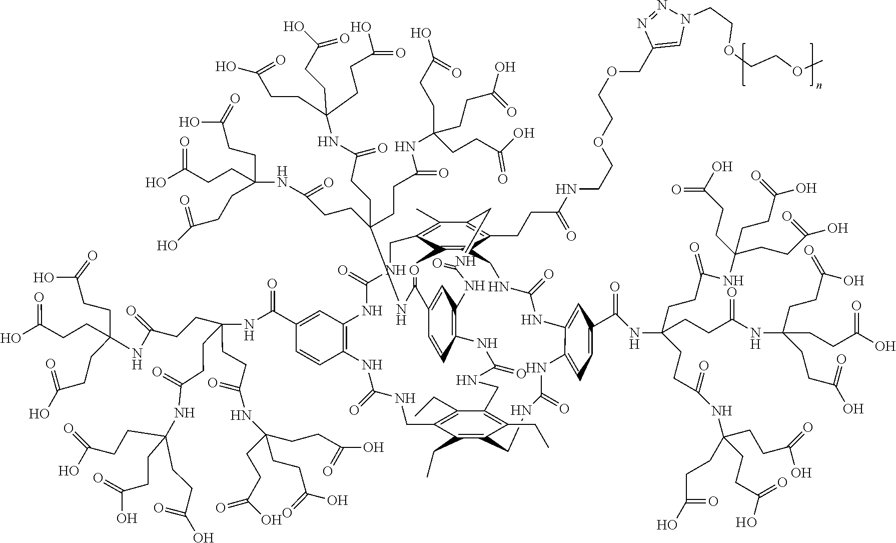

Prepared according to General Procedure B from GI54 (2 mg, 0.450 μmol), 40 kDa mPEG azide (15 mg, 0.375 μmol), sodium ascorbate (1.5 mg, 7.50 μmol), CuSO₄·5H₂O (0.5 mg, 1.90 μmol) and NaHCO₃ (1 mg, 10.1 μmol) in water (0.7 mL).

Product obtained by dialysis (10k MWCO) of reaction mixture, followed by lyophilisation to give a white powder (16 mg, 0.364 μmol, 97%).

Absₘₐₓ=270 nm

### Example 32: Synthesis of GBM32

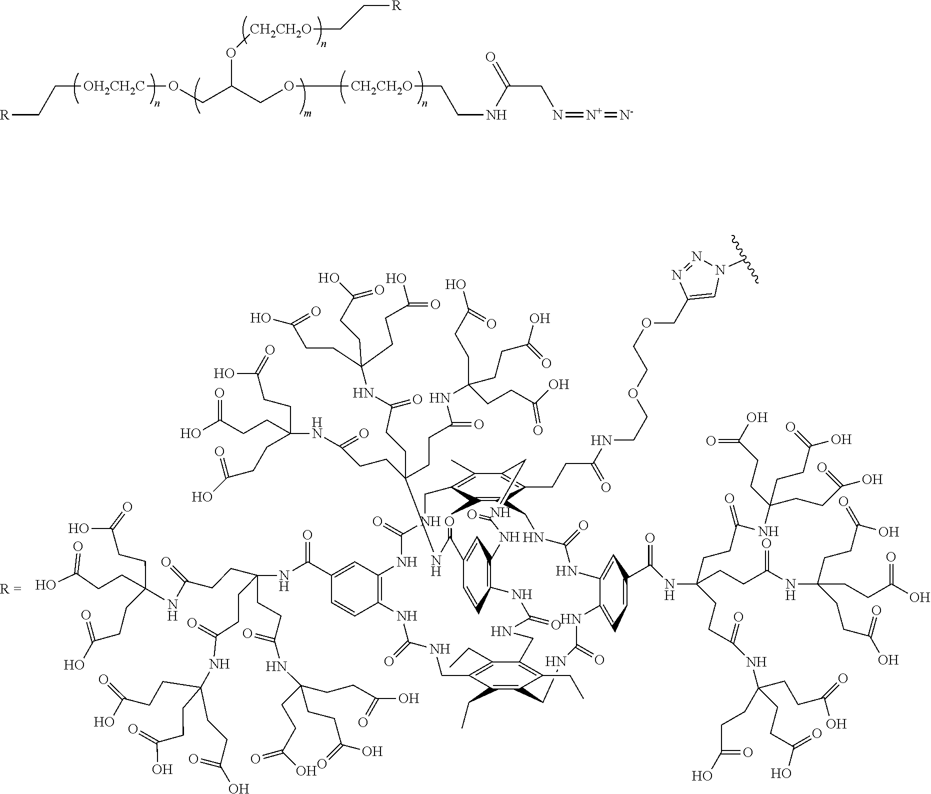

Prepared according to General Procedure B from GI54 (6.0 mg, 1.5 μmol), 8-arm PEG-azide(7)/amine(1) (8.0 mg, 0.200 μmol), sodium ascorbate (0.8 mg, 4.0 μmol), CuSO₄·5H₂O (0.2 mg, 1.0 μmol) and NaHCO₃ (1 mg, 12.0 μmol) in water (0.7 mL).

Azidoacetic acid NHS ester (1.0 mg, 5.0 μmol) added to reaction and stirred for 6 hours. Product obtained by dialysis (10k MWCO) of reaction mixture followed by lyophilisation to give a white powder (10.0 mg, 0.147 μmol, 74%).

Absₘₐₓ=270 nm

### Example 33: Synthesis of GBM33

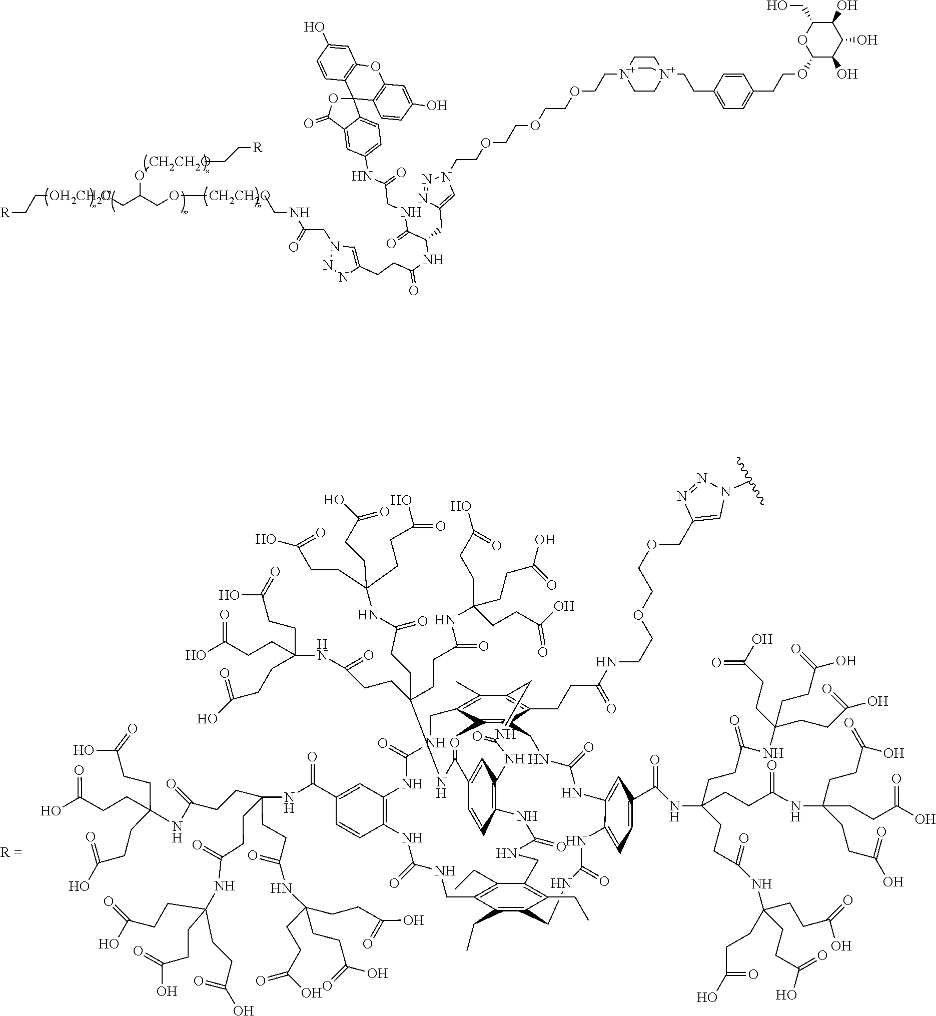

Prepared according to General Procedure B from GBM32 (5.5 mg, 0.084 μmol), I39 (15 mg, 0.375 μmol), sodium ascorbate (0.6 mg, 3.2 μmol), CuSO₄·5H₂O (0.2 mg, 1.0 μmol) and NaHCO₃ (1 mg, 12.0 μmol) in water (0.7 mL). Product obtained by dialysis (10k MWCO) of reaction mixture in 150 μM glucose solution. Subsequent solution was further purified through repeated centrifugation through a 30,000 MWCO membrane, diluting with 50 μM D-glucose, followed by water. Lyophilisation to give a yellow solid (5.8 mg, 0.087 μmol, 100%).

Absₘₐₓ=480 nm, EMₘₐₓ=530 nm

### Example 34: Synthesis of GBM34

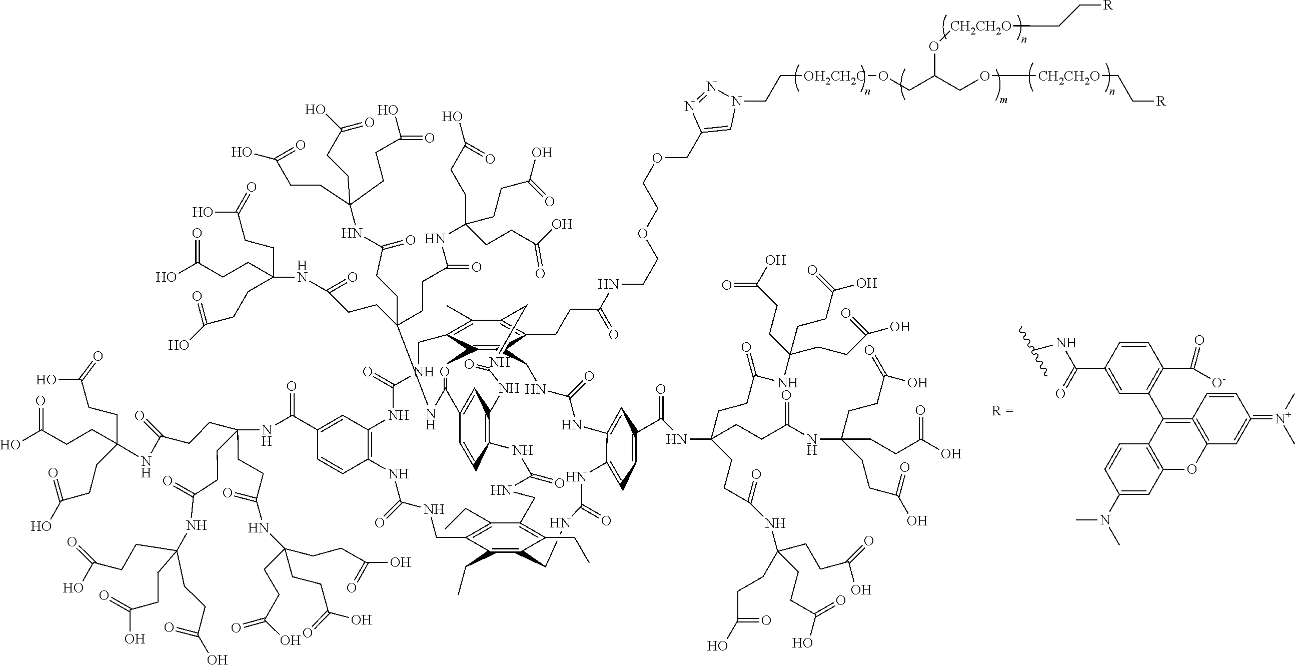

8-Arm PEG-Azide/Amine, Azide(1)/Amine(7) 40k (20 mg, 0.500 μmol) and NaHCO₃ (10 mg, 119 μmol) dissolved in THF (0.5 mL) and water (20 μL). TAMRA succinic ester (2.0 mg, 3.9 μmol) added and stirred for 2 hours. GI54 (2.2 mg, 0.550 μmol) added, followed by a solution of sodium ascorbate (2.0 mg, 10.0 μmol) and CuSO₄·5H₂O (0.6 mg, 2.5 μmol) in water (0.5 mL) and stirred for 16 hours. Product obtained by dialysis (10k MWCO) of reaction mixture followed by lyophilisation to give a purple powder (10.0 mg, 0.147 μmol, 74%).

Absₘₐₓ=550 nm, EMₘₐₓ=575 nm

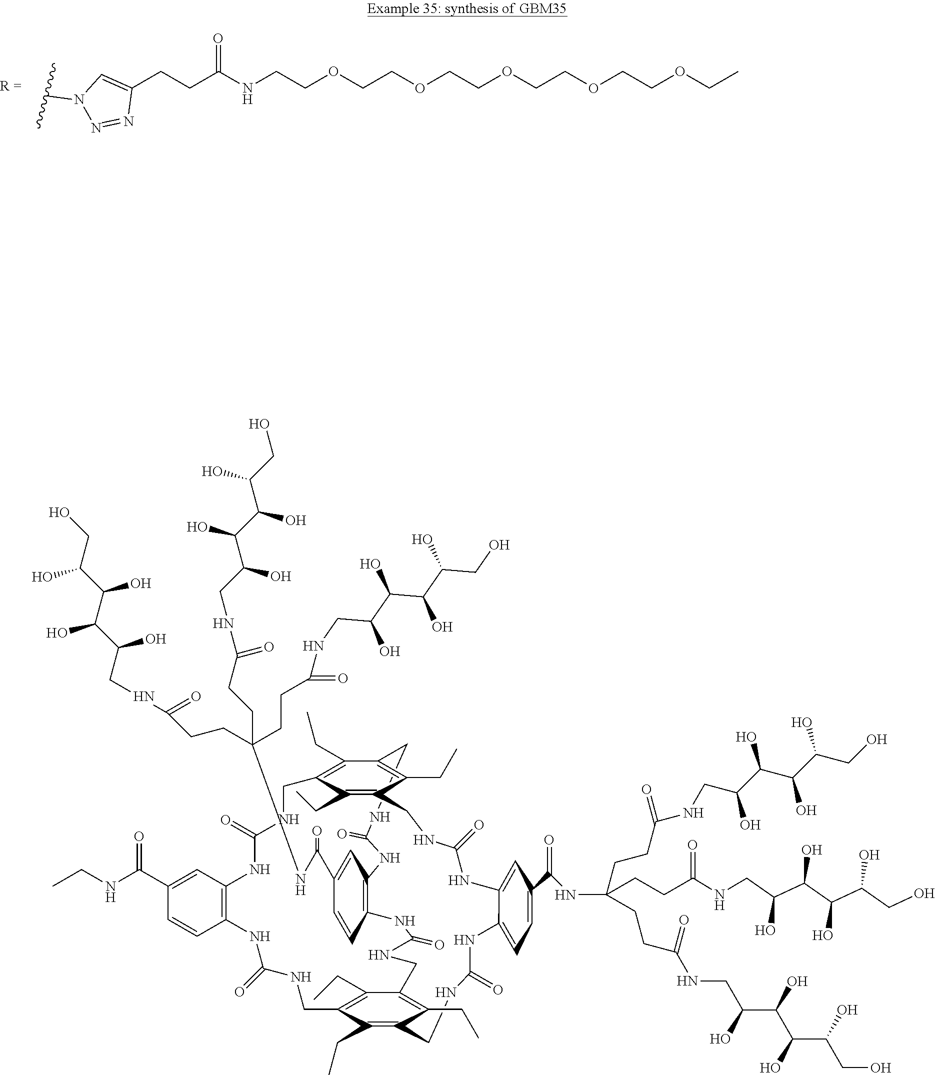

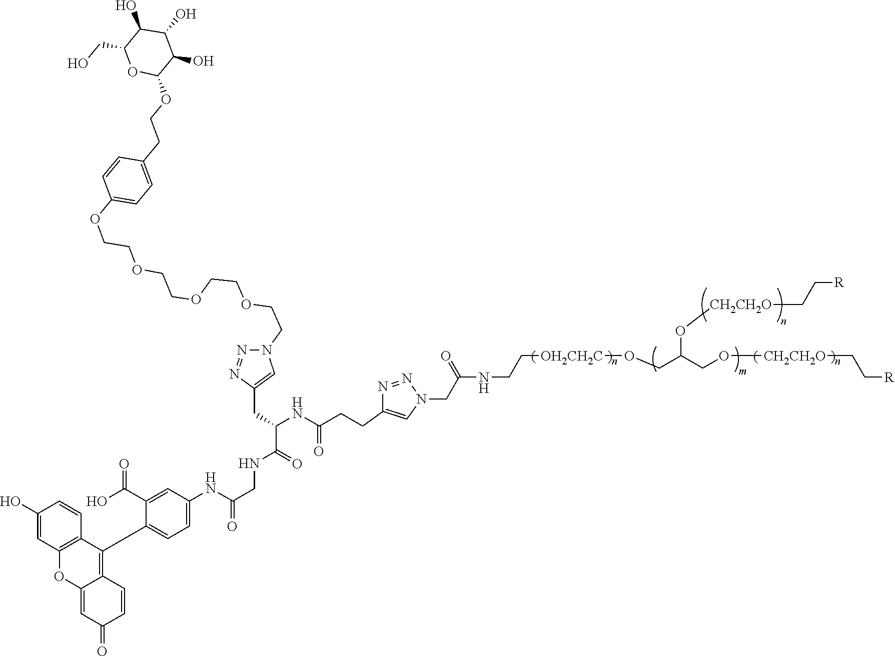

Prepared according to General Procedure B from GI60 (6 mg, 0.098 μmol), FIM1 (0.2 mg, 0.196 μmol), sodium ascorbate (0.2 mg, 0.98 μmol), CuSO₄·5H₂O (0.1 mg, 0.20 μmol). Product obtained by repeated centrifugation through a 30,000 MWCO membrane, diluting with 50 μM D-glucose, followed by water. Lyophilisation to give a yellow powder (6 mg, 0.069 μmol, 70%).

Absₘₐₓ=480 nm, EMₘₐₓ=530 nm

### Example 36: Synthesis of GBM36

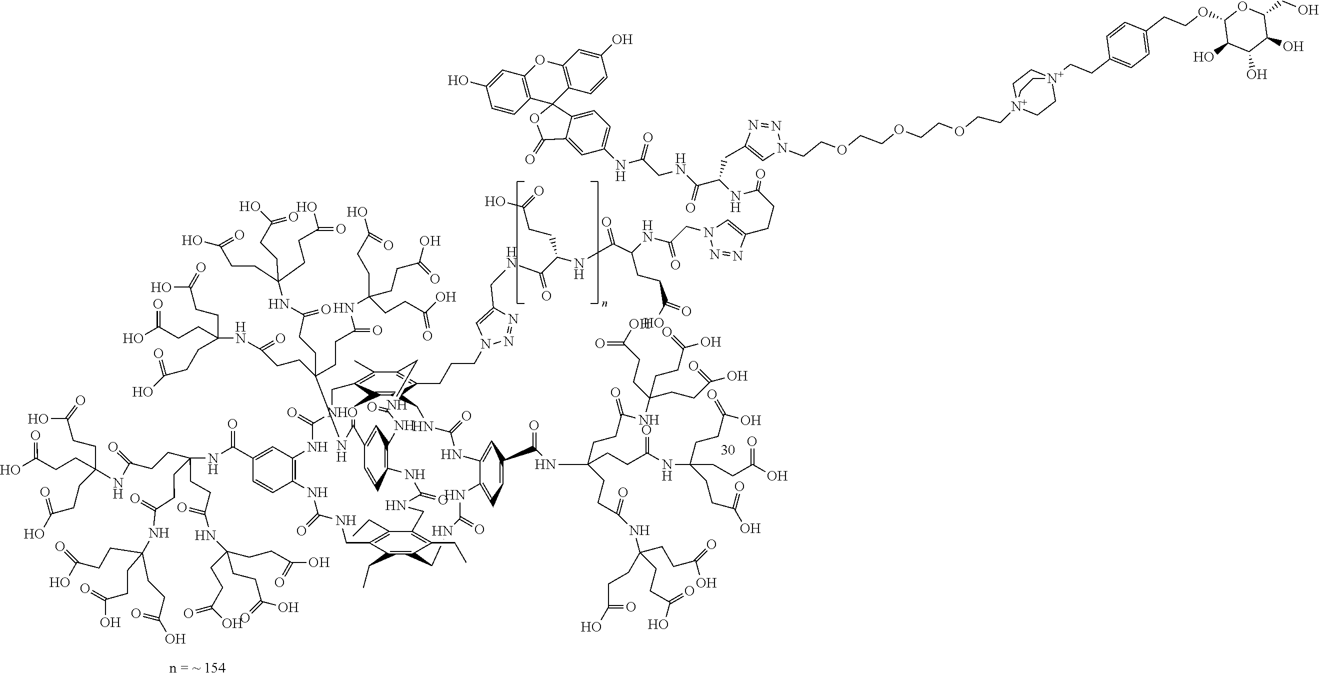

Prepared according to General Procedure B from GI46 (3.2 mg, 0.825 μmol), Alkyne-Poly(L-Glu) (154) (15 mg, 0.750 μmol), sodium ascorbate (3 mg, 15.0 μmol), CuSO₄·5H₂O (1 mg, 3.8 μmol) and NaHCO₃ (12 mg, 142.8 μmol) in water (1 mL). After stirring for 16 h, Azidoacetic acid NHS ester (3 mg, 15.0 μmol) was added as a solution in MeCN (0.4 mL) and stirred for 6 hours. Reaction mixture was dialysed (6K MWCO) and half the dialysed mixture was treated according to General Procedure B with 139 (0.5 mg, 0.375 μmol), sodium ascorbate (1.5 mg, 7.5 μmol) and CuSO₄·5H₂O (0.5 mg, 1.9 μmol). Product obtained by repeated centrifugation through a 10,000 MWCO membrane, washing sequentially with 50 uM D-glucose and water. Lyophilisation to give a yellow powder (1.5 mg, 0.060 μmol, 16%).

Absₘₐₓ=480 nm, EMₘₐₓ=530 nm

### Example 37: Synthesis of GBM37

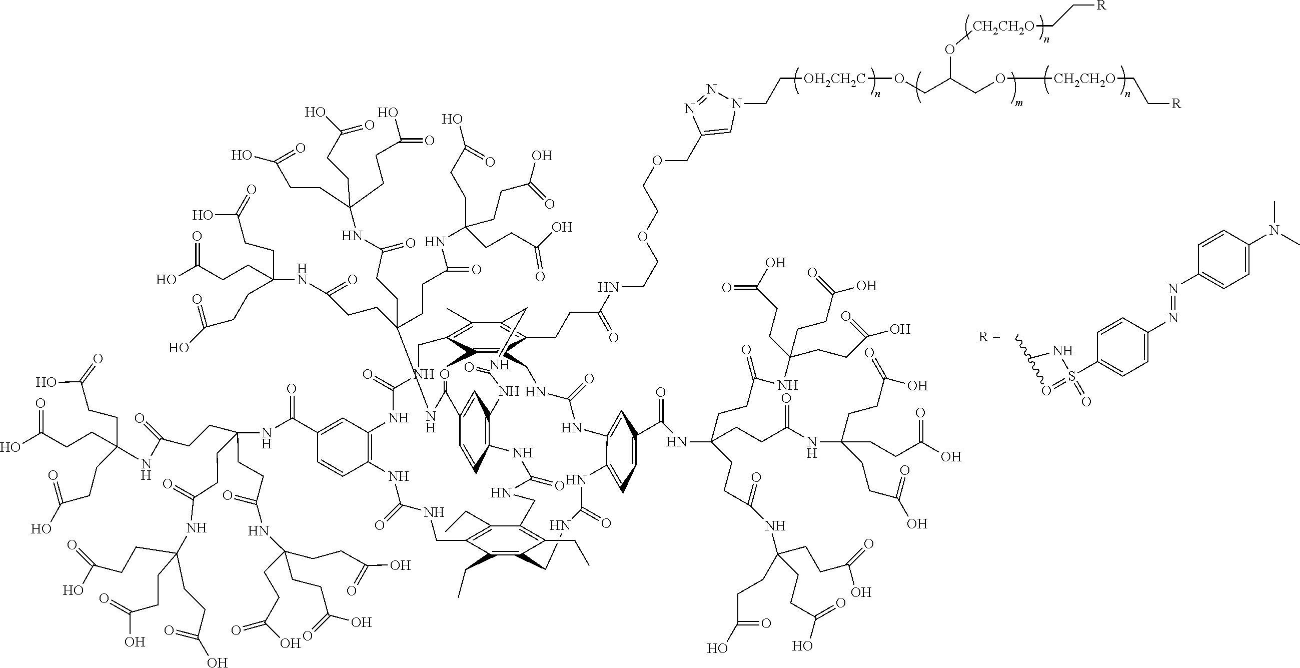

8-Arm PEG-Azide/Amine, Azide(1)/Amine(7) 40k (20 mg, 0.500 μmol) and NaHCO₃ (5 mg, 59.5 μmol) dissolved in THF (0.5 mL) and water (20 μL). Dabsyl Chloride (1.2 mg, 3.9 μmol) added and stirred for 2 hours. GI54 (2.2 mg, 0.550 μmol) added, followed by a solution of sodium ascorbate (2.0 mg, 10.0 μmol) and CuSO₄·5H₂O (0.6 mg, 2.5 μmol) in water (0.5 mL) and stirred for 16 hours. Product obtained by dialysis (10k MWCO) of reaction mixture followed by lyophilisation to give an orange powder (20.0 mg, 0.452 μmol, 90%).

Absₘₐₓ=450 nm

### Example 38: Synthesis of GBM38

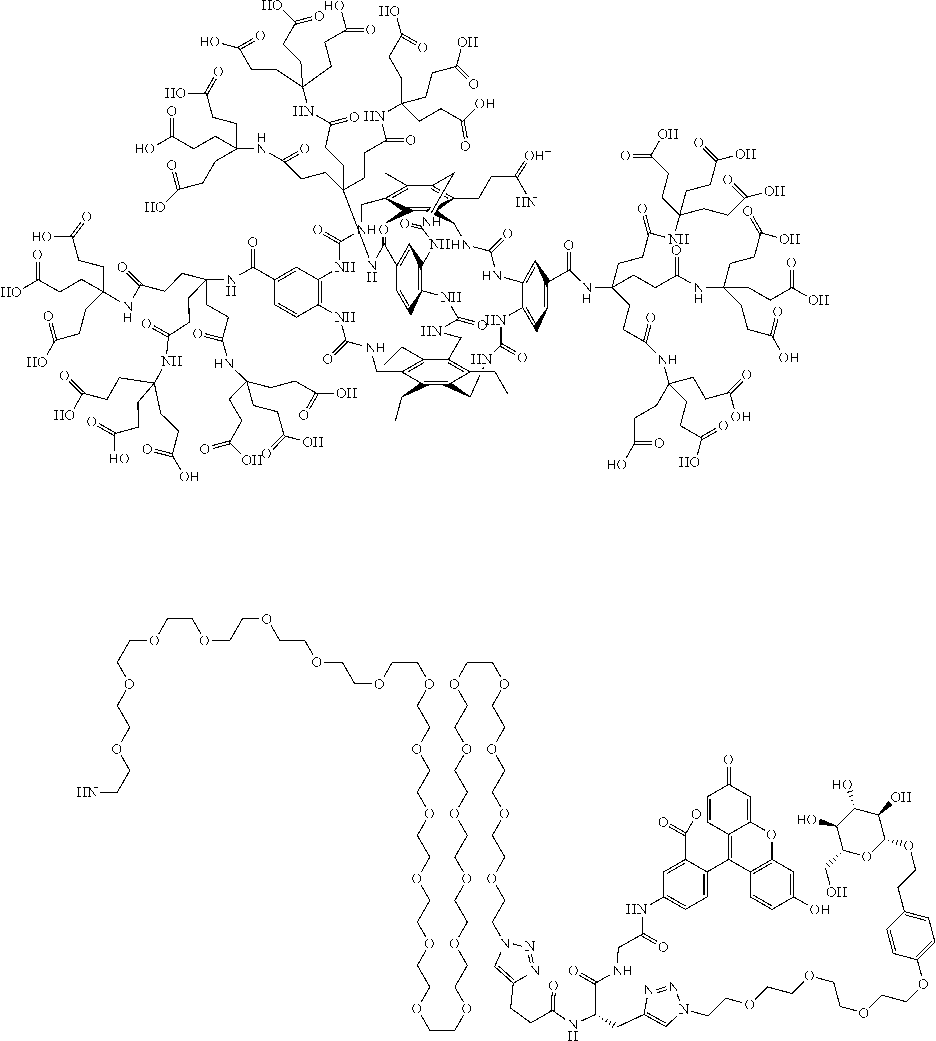

Prepared according to General Procedure B from GI82 (7 mg, 1.1 μmol), FIM1 (2 mg, 1.3 μmol), sodium ascorbate (1 mg, 5.0 μmol), CuSO₄·5H₂O (0.3 mg, 1.3 μmol) in water (2 mL) and THF (1 mL). Solvents removed under a stream of N₂ and the residue treated with TFA (0.2 mL) in DCM (0.5 mL). The acid was quenched with sat. aq. NaHCO₃ and the resultant solution purified by dialysis (1 kDa MWCO) in water (1.8 L), and resultant solution lyophilised to give the product as an orange powder (6 mg, 0.99 μmol, 90%)

Absₘₐₓ=480 nm. Emₘₐₓ=520 nm.

### Example 39: Synthesis of GBM39

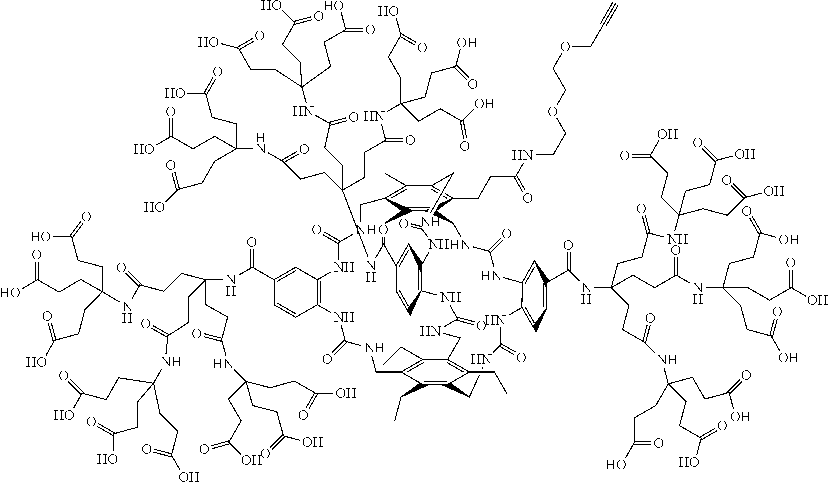

This molecule is also referred to herein as GI54. See synthesis of GI54 below for synthesis.

### Example 40: Synthesis of GBM40

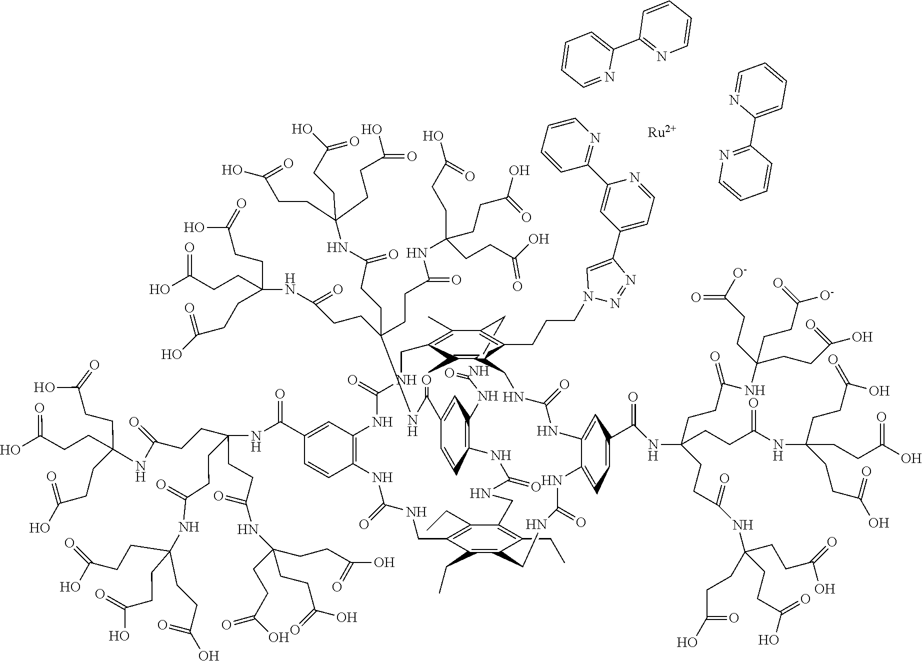

Prepared according to General Procedure B from GI48 (14 mg, 3.6 μmol), I110 (3.6 mg, 5.4 μmol), CuSO₄·5H₂O (0.4 mg, 1.4 μmol), sodium ascorbate (2.1 mg, 10.8 μmol) and NaHCO₃ (15 mg, 180 μmol) in water (0.5 mL). Purified by RP MPLC (H₂O with increasing MeCN (0.1% formic acid)). Product obtained as an orange solid (12.0 mg, 2.7 μmol, 74%).

MS (electrospray, +ve), [M+3H]³⁺ calculated for C₂₀₈H₂₆N₃₃O₇₂Ru requires: 1494.5806, found: 1494.4.

### Example 41: Synthesis of GBM41

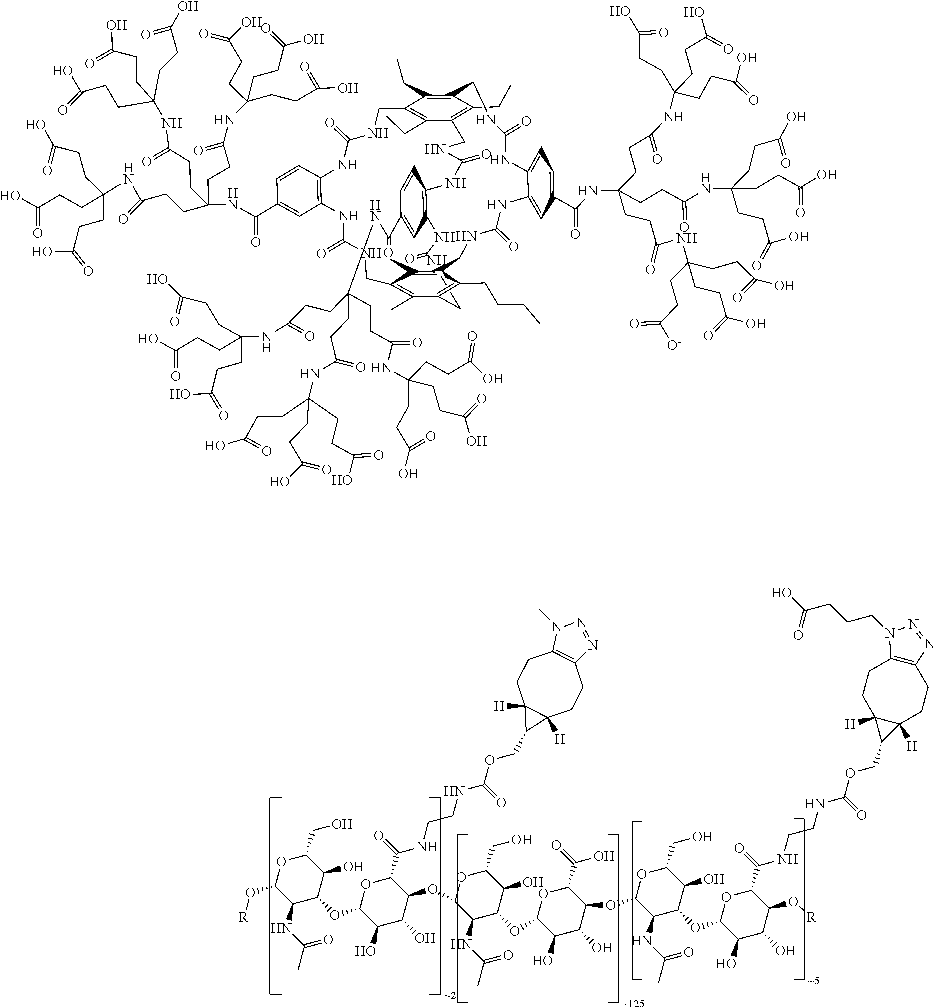

GI48 (3 mg, 0.76 μmol) and GI86 (19 mg, 0.38 μmol) were dissolved in water (1.5 mL) with NaHCO₃ (2 mg, 22.8 μmol) and stirred for 4 h. 4-azidobutanoic acid (1 mg, 7.7 μmol) was added and the reaction stirred for a further 2 h. The reaction mixture was dialysed (6000 MWCO) and the resultant solution freeze dried to give the product as a white solid (20 mg, 0.36 μmol, 95%).

Absₘₐₓ=270 nm

### Example 42: Synthesis of GBM42

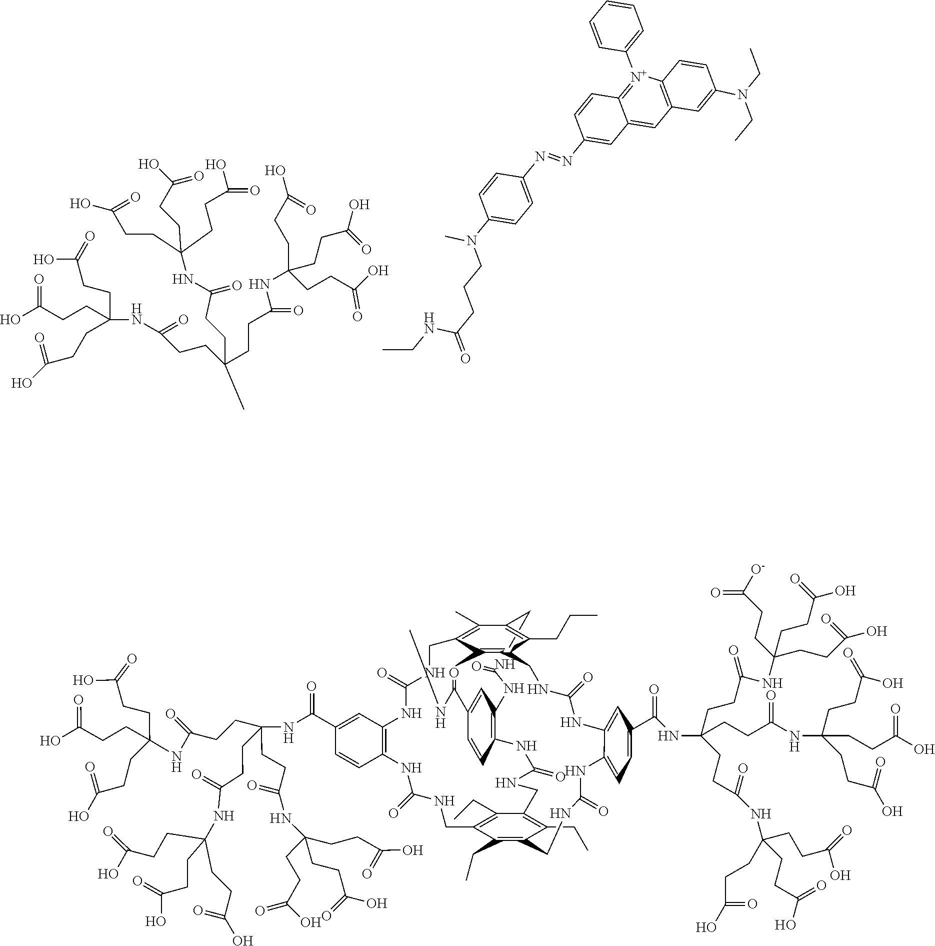

GI87 (10 mg, 1.9 μmol) was dissolved in THF (0.5 mL) and BHQ-3 Carboxylic Acid, Succinimidyl Ester (2 mg, 2.8 μmol) was added and stirred together for two hours. Solvents were then removed under a stream of N₂. The residue was treated according to General Procedure D in TFA (0.3 mL) and DCM (0.8 mL). Purified by RP MPLC (H₂O with increasing MeCN (0.1% formic acid)). Product obtained as a blue solid (7.0 mg, 1.6 μmol, 89%).

MS (electrospray, +ve), [M+3H]³⁺ calculated for C₂₀₉H₂₃₀N₃₁O₇₃ requires: 1464.6401, found: 1464.6.

### Example 43: Synthesis of GBM43

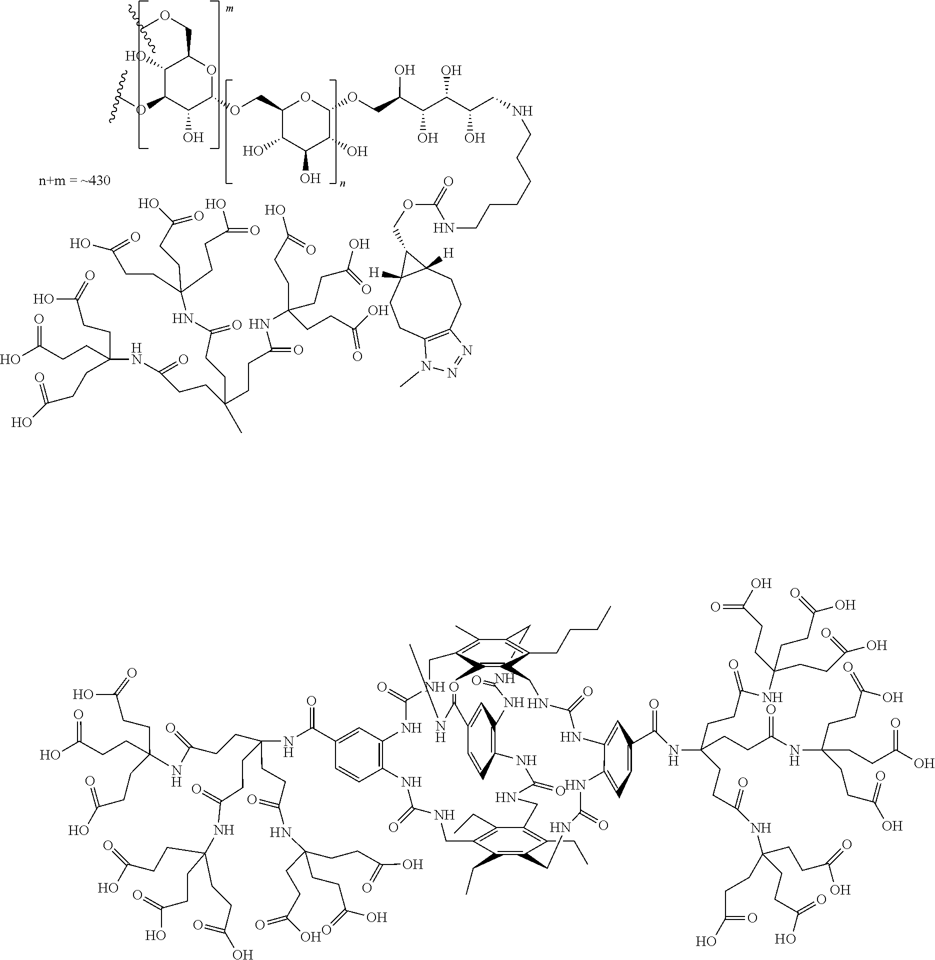

GI48 (5 mg, 1.4 μmol) and GI88 (35 mg, 0.5 μmol) were dissolved in water (1 mL) with NaHCO₃ (3.5 mg, 41.4 μmol) and stirred for 18 h. The reaction mixture was dialysed (6000 MWCO) and the resultant solution freeze dried to give the product as a white solid (33 mg, 0.45 μmol, 90%).

Absₘₐₓ=270 nm

### Example 44: Synthesis of GBM44

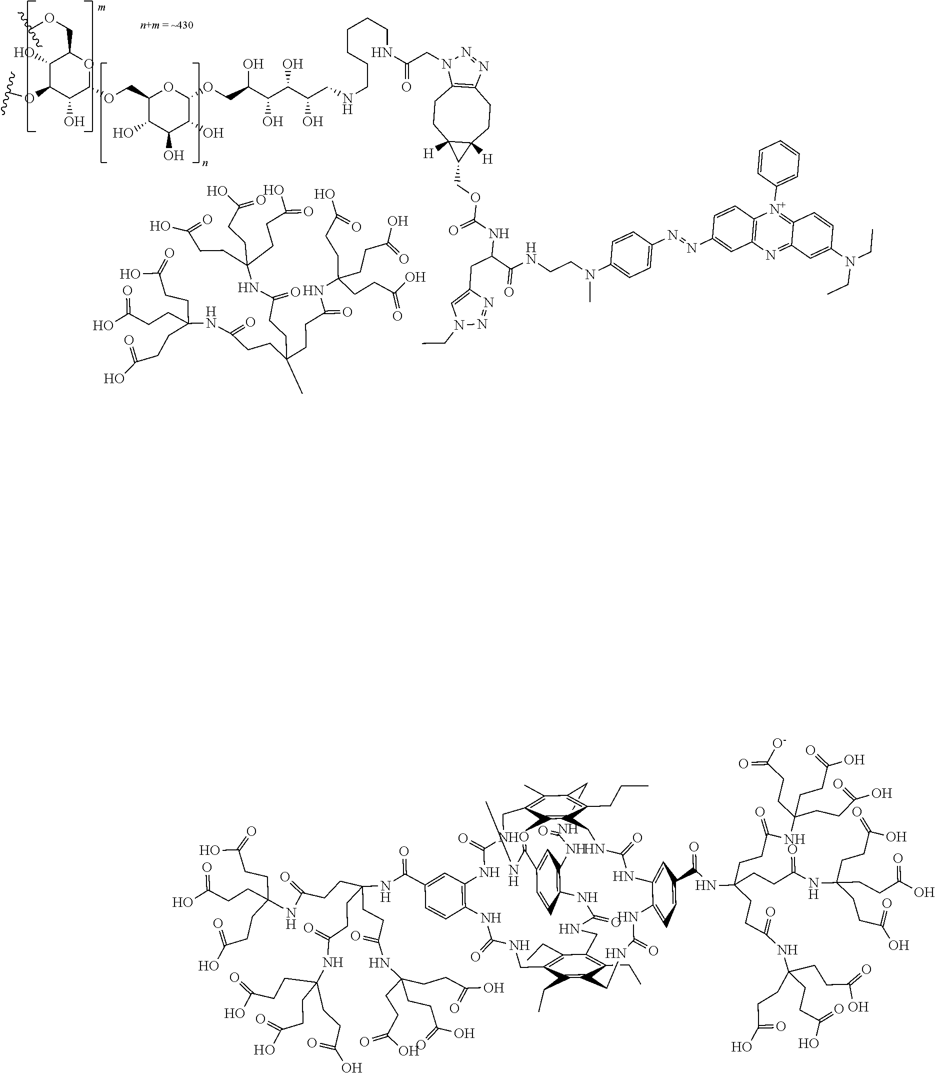

GI89 (0.4 mg, 0.08 μmol) and I115 (5 mg, 0.7 μmol) were dissolved in water with 0.02% 2-chloroacetamide (1 mL) and stirred for 20 h. The reaction mixture was dialysed (6000 MWCO) and the resultant solution freeze dried to give the product as a blue solid (5 mg, 0.067 μmol, 85%).

Absₘₐₓ=270, 610 nm

### Glucose Binding Molecule (GBM) Intermediates (GIX) Synthetic Procedures

### Synthesis of GI1

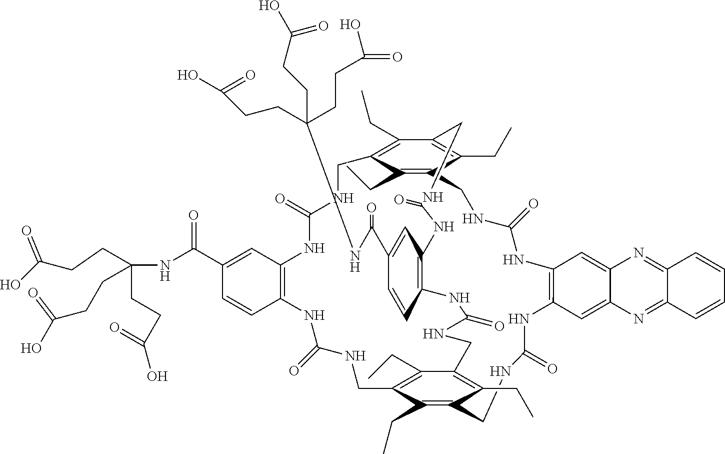

Prepared according to General Procedure D from GI2 (40 mg, 0.020 mmol) in TFA (2 mL) and DCM (2 mL). Purified by RP MPLC (H₂O with increasing MeCN (0.1% formic acid)) to give the product as an orange solid (26 mg, 0.016 mmol, 78%).

¹H NMR (400 MHz, DMSO-d₆) δ 8.63 (d, J=10.9 Hz, 2H), 8.56-8.45 (m, 1H), 8.26 (s, 1H), 8.15-8.07 (m, 2H), 8.04 (s, 1H), 7.97-7.84 (m, 5H), 7.81-7.74 (m, 2H), 7.61-7.53 (m, 2H), 7.48-7.34 (m, 3H), 7.31 (s, 2H), 6.49-6.40 (m, 3H), 6.38-6.25 (m, 3H), 4.49-4.10 (m, 12H), 2.86-2.54 (m, 12H), 2.21-2.05 (m, 12H), 2.02-1.84 (m, 12H), 1.25-0.99 (m, 18H).

MS (electrospray, +ve), [M+H]⁺ calculated for C₈₂H₉₉N₁₆O₂₀ requires: 1627.722, found: 1627.6

### Synthesis of GI2

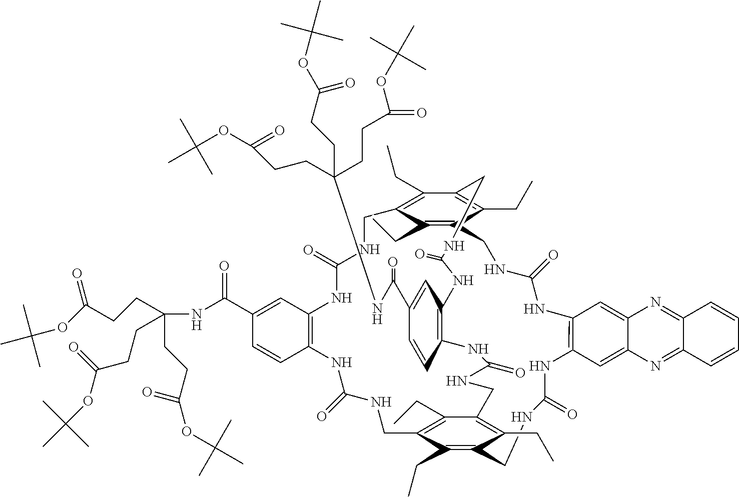

Prepared according to General Procedure E from GI3 (83 mg, 0.05 mmol) and “TEB_NCO” (synthesised according to compound 103 of WO 2018/167503, incorporated herein by reference) (20 mg, 0.06 mmol) in pyridine (11 mL), DMF (5 mL) and DCM (1.6 mL). Purified by RP MPLC (H₂O with increasing acetone) to give the product as an orange solid (40 mg, 0.020 mmol, 40%).

¹H NMR (400 MHz, DMSO-d₆+1% D2O) δ 8.58-8.43 (m, 2H), 8.12-8.02 (m, 2H), 7.95-7.70 (m, 4H), 7.50-7.29 (m, 4H), 4.44-4.07 (m, 12H), 2.75-2.52 (m, 12H), 2.14-1.99 (m, 12H), 1.94-1.79 (m, 12H), 1.29 (s, 54H), 1.17-1.01 (m, 18H).

### Synthesis of GI3

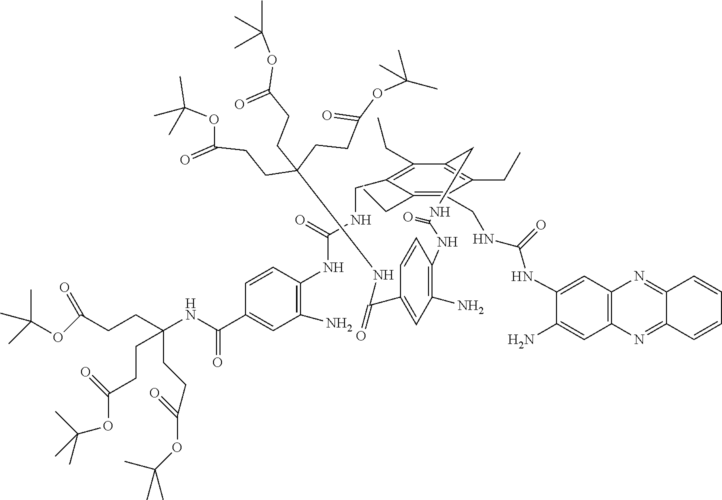 [SMILES: CCc1c(CNC(=O)Nc2ccc(C(=O)NC(CCC(=O)OC(C)(C)C)(CCC(=O)OC(C)(C)C)CCC(=O)OC(C)(C)C)cc2N)c(CC)c(CNC(=O)Nc2cc3nc4ccccc4nc3cc2N)c(CC)c1CNC(=O)Nc1ccc(C(=O)NC(CCC(=O)OC(C)(C)C)(CCC(=O)OC(C)(C)C)CCC(=O)OC(C)(C)C)cc1N]

GI4 (160 mg, 0.077 mmol) dissolved in DMF (2.4 mL) and piperidine (0.24 mL, 2.46 mmol) added. Stirred at RT for 20 minutes. Purified by RP MPLC (H₂O with increasing acetone) to give the product as an orange solid (80 mg, 0.049 mmol, 64%).

¹H NMR (400 MHz, DMSO-d₆) δ 8.57 (s, 1H), 8.24 (s, 1H), 8.03 (d, J=8.4 Hz, 1H), 7.99 (d, J=7.9 Hz, 1H), 7.77-7.63 (m, 4H), 7.58 (d, J=8.2 Hz, 2H), 7.20-7.11 (m, 6H), 7.03 (d, J=8.8 Hz, 3H), 6.67 (s, 1H), 6.35 (s, 2H), 5.98 (s, 2H), 4.72 (s, 4H), 4.47 (s, 2H), 4.39 (s, 4H), 2.98-2.73 (m, 6H), 2.21-2.06 (m, 12H), 2.04-1.84 (m, 12H), 1.37 (s, 54H), 1.29-1.16 (m, 9H)).

MS (electrospray, +ve), [M+H]⁺ calculated for C₃₃H₁₂₆N₁₃O₁₇ requires: 1636.939, found: 1636.8

### Synthesis of GI4

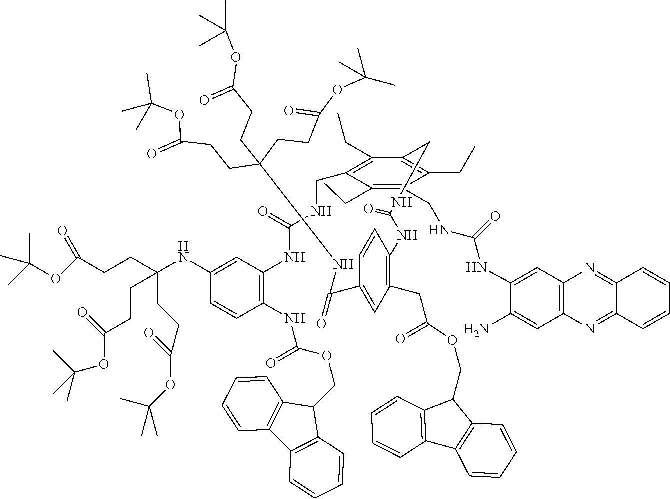 [SMILES: CCc1c(CNC(=O)Nc2cc3nc4ccccc4nc3cc2N)c(CC)c(CNC(=O)Nc2cc(NC(CCC(=O)OC(C)(C)C)(CCC(=O)OC(C)(C)C)CCC(=O)OC(C)(C)C)ccc2NC(=O)OCC2c3ccccc3-c3ccccc32)c(CC)c1CNC(=O)Nc1ccc(C(=O)NC(CCC(=O)OC(C)(C)C)(CCC(=O)OC(C)(C)C)CCC(=O)OC(C)(C)C)cc1CC(=O)OCC1c2ccccc2-c2ccccc21]

“TEB NCO” (synthesised according to compound 103 of WO 2018/167503, incorporated herein by reference) (80 mg, 0.244 mmol) and “G1M linker” (synthesised according to compound 2 of WO 2018/167503, incorporated herein by reference) (377 mg, 0.489 mmol) dissolved in anhydrous pyridine and stirred at RT overnight. Solution of 2,3-diaminophenazine (308 mg, 1.466 mmol) in DMF/pyridine (1:1, 4 mL) at 80° C. was added and stirred for 2 hours. Removed solvent under vacuum, redissolved crude in acetone, precipitated with 1 M HCl and resultant precipitate isolated by centrifugation. Product was purified by RP MPLC (H₂O with increasing acetone) to give the product as an orange solid (160 mg, 0.077 mmol, 32%).

¹H NMR (400 MHz, DMSO-d₆) δ 8.94 (s, 1H), 8.25-8.01 (m, 5H), 8.01-7.90 (m, 2H), 7.83-7.70 (m, 6H), 7.71-7.52 (m, 6H), 7.39-7.24 (m, 5H), 7.16 (s, 6H), 6.99 (s, 2H), 6.59 (s, 1H), 5.92 (s, 1H), 4.67-3.85 (m, 12H), 2.91-2.58 (m, 6H), 2.19-2.00 (m, 12H), 2.00-1.75 (m, 12H), 1.32 (s, 54H), 1.23-0.95 (m, 9H).

### Synthesis of GI5

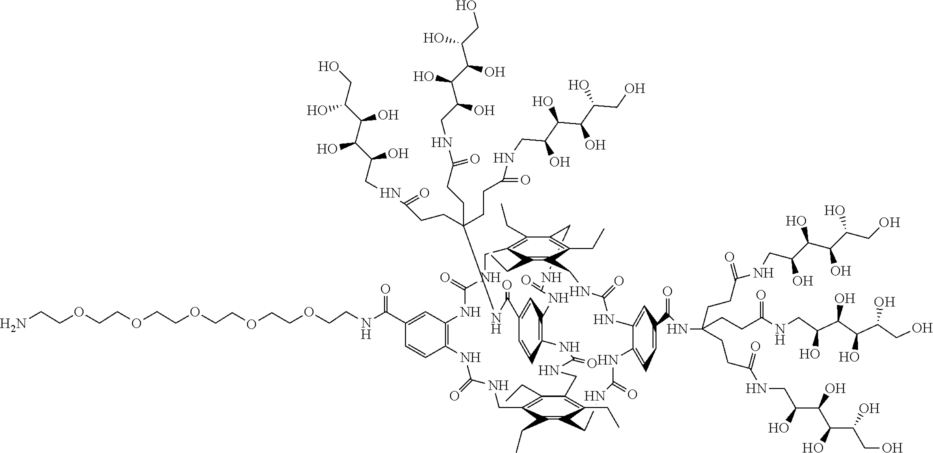

To a solution of GI6 (20.0 mg, 0.007 mmol) in H₂O (1.2 mL) and THF (0.1 mL) was added Pd/C (6.0 mg, 10% w/w). Reaction stirred overnight under an atmosphere of hydrogen at RT. Filtered through celite with THF/H₂O and concentrated. Product obtained as a white solid (18.5 mg, 0.007 mmol, 93%).

MS (electrospray, +ve) [M+2H]²⁺ calculated for C₁₂₅H₂₀₂N₂₂O₅₀ requires: 1406.1982, found: 1406.2

### Synthesis of GI6

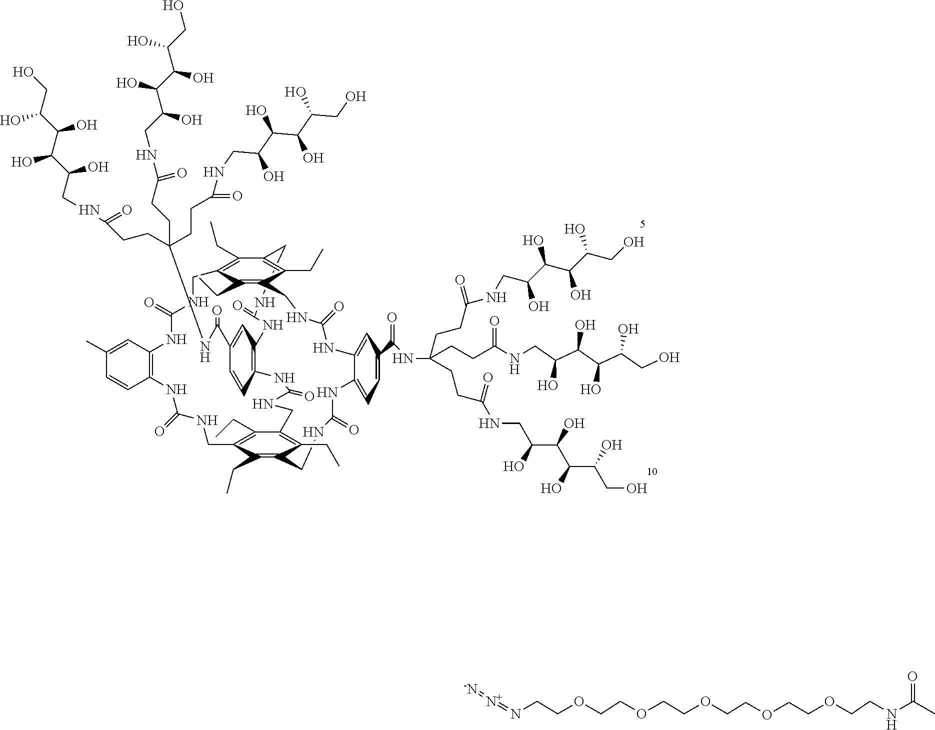

Prepared according to General Procedure A from GI56 (120 mg, 0.040 mmol), azido-PEG₅-amine (18 mg, 0.059 mmol), HBTU (36 mg, 0.095 mmol), HOBt·H₂O (15 mg, 0.098 mmol) and DIPEA (28 μL, 0.160 mmol) in pyridine (1.0 mL). Purified by RP MPLC (H₂O with increasing MeCN). Product obtained as a white solid (72.0 mg, 0.025 mmol, 63%).

MS (electrospray, +ve) [M+3H]³⁺ calculated for C₁₂₂H₂₀₁N₂₄O₅₀ requires: 946.4647, found: 946.4.

### Synthesis of GI7

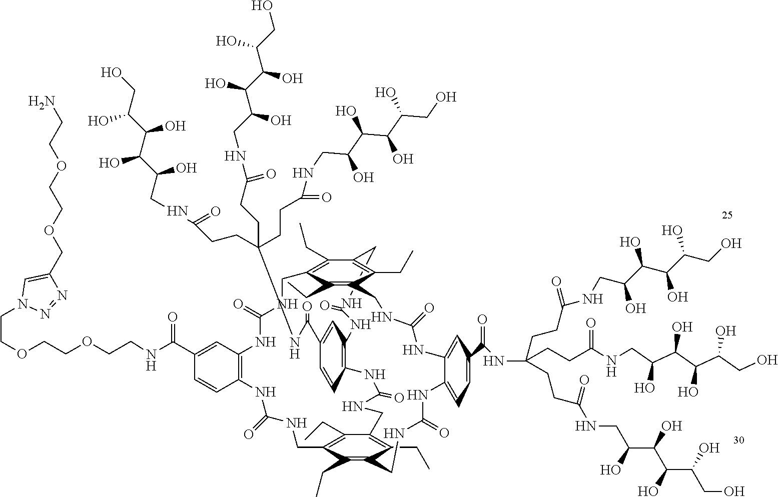

Prepared according to General Procedure B from GI8 (352 mg, 0.121 mmol), 2-[2-(2-propynyloxy)ethoxy]ethylamine (35.0 mg, 0.244 mmol), CuSO₄·5H₂O (20 mg, 0.080 mmol), sodium ascorbate (17.0 mg, 0.086 mmol) in THF (1.6 mL) and water (1.6 mL). Precipitated product with acetone and isolated by centrifugation. Product obtained as a pale yellow solid (345 mg, 0.121 mmol, 100%).

MS (electrospray, +ve) [M+3H]³⁺ calculated for C₁₂₆H₂₀₂N₂₅O₄₉ requires: 950.1367, found: 950.1

### Synthesis of GI8

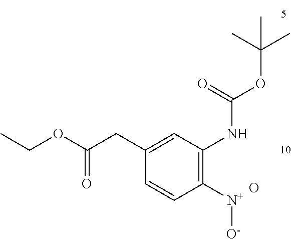

Prepared according to General Procedure A from GI56 (160 mg, 0.053 mmol), azido-PEG₂-amine (19 mg, 0.109 mmol), HBTU (48 mg, 0.127 mmol), HOBt·H₂O (19.0 mg, 0.124 mmol) and DIPEA (38 μL, 0.217 mmol) in pyridine (1.4 mL). Purified by RP MPLC (H₂O with increasing MeCN). Product obtained as a white solid (89 mg, 0.033 mmol, 62%).

MS (electrospray, +ve) [M+3H]³⁺ calculated for C₁₂₆H₂₀₂N₂₅O₄₉ requires: 902.4385, found: 902.4

### Synthesis of GI9

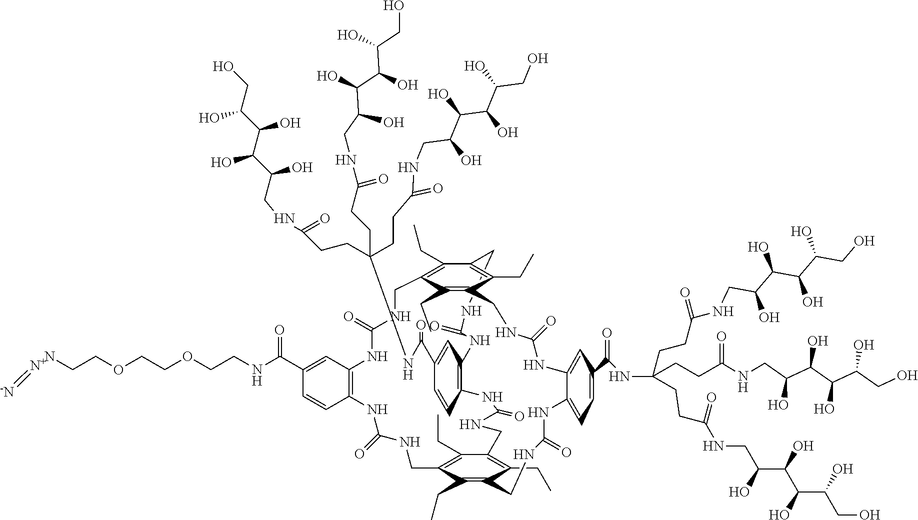

A solution of Boc₂O (3.75 g, 17.2 mmol) in DCM (25 mL) via a dropping funnel, was added to a solution of ethyl 2-(4-amino-3-nitrophenyl)acetate (3.5 g, 15.6 mmol), Et₃N (2.2 mL, 15.6 mmol) and DMAP (1.05 g, 8.59 mmol) in DCM (30 mL). After 1 hour the organic solvent was removed under reduced pressure then the reaction mixture was partitioned between EtOAc and 1 M HCl. The organic solvent was removed under reduced pressure and the dark brown oil was purified by MPLC (DCM with increasing EtOAc). Product isolated as a yellow oil (2.5 g, 7.71 mmol, 49%)

¹H NMR (400 MHz, Chloroform-d) δ 9.62 (s, 1H), 8.52 (d, J=8.8 Hz, 1H), 8.12 (d, J=2.1 Hz, 1H), 7.53 (dd, J=8.8, 2.2 Hz, 1H), 4.16 (q, J=7.1 Hz, 2H), 3.62 (s, 2H), 1.54 (s, 9H), 1.26 (t, J=7.1 Hz, 3H).

### Synthesis of GI10

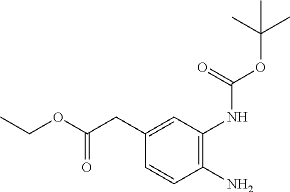 [SMILES: CCOC(=O)Cc1ccc(N)c(NC(=O)OC(C)(C)C)c1]

A solution of GI9 (295 mg, 0.91 mmol) inEtOH (10 mL) was added to a slurry of Pd/C (37 mg, 10% w/w) in DCM (1 mL), and this was stirred vigorously overnight under an atmosphere of hydrogen. The slurry was filtered through a bed of celite, and the solvent was removed under reduced pressure to give a gum which was purified by MPLC (petrol with increasing EtOAc) to give the product as a pale pink oil (236 mg, 0.802 mmol, 88%).

¹H NMR (400 MHz, Chloroform-d) δ 7.20 (d, J=8.0 Hz, 1H), 6.74-6.67 (m, 2H), 6.22 (s, 1H), 4.12 (q, J=7.1 Hz, 2H), 3.49 (s, 2H), 1.50 (s, 10H), 1.24 (t, J=7.1 Hz, 3H).

### Synthesis of GI11

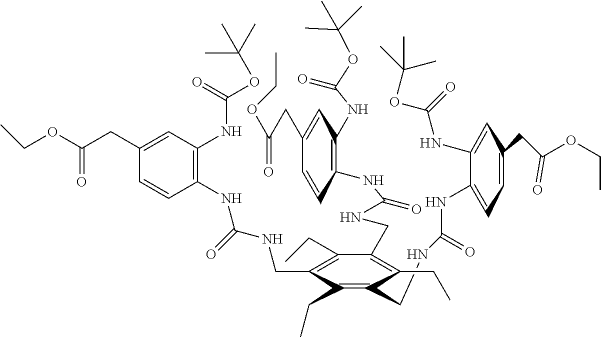

To a solution of GI10 (226 mg, 0.769 mmol) in dry DCM (4 mL), was added “TEB NCO” (synthesised according to compound 103 of WO 2018/167503, incorporated herein by reference) (74 mg, 0.226 mmol) as a solid, followed by pyridine (0.1 mL). After stirring overnight, a thick white precipitate had formed. To this Et₂O (20 mL) was added and after sonication and stirring the solid was collected by filtration. The solid was resuspended in Et₂O (10 mL) and collected by filtration before drying to give the product as a white powder (265 mg, 0.219 mmol, 97%).

¹H NMR (400 MHz, DMSO-d₆) δ 8.30 (s, 1H), 7.88 (s, 1H), 7.72 (s, 1H), 7.18 (d, J=8.2 Hz, 1H), 6.83 (dd, J=8.5, 2.0 Hz, 1H), 6.77 (s, 1H), 4.36 (s, 2H), 4.07 (q, J=7.1 Hz, 2H), 3.57 (s, 2H), 2.80 (d, J=7.9 Hz, 2H), 1.40 (s, 8H), 1.18 (t, J=7.1 Hz, 4H).

### Synthesis of GI12

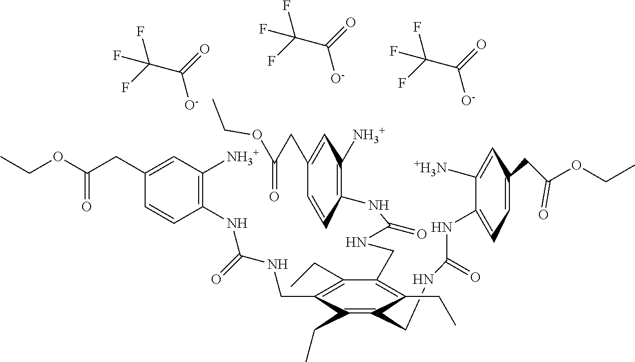

To GI11 (260 mg, 0.215 mmol) was added a mixture of DCM (3 mL) and TFA (3 mL) which gave a solution. After 2 hours the reaction mixture was concentrated partially then diluted with Et₂O (20 mL) sonicated and stirred, then the solid was collected by filtration. The solid was resuspended in Et₂O (10 mL) and collected by filtration before drying to give the product as a white powder (252 mg, 0.207 mmol, 97%)

¹H NMR (400 MHz, DMSO-d₆) δ 7.94 (s, 3H), 7.23 (d, J=1.9 Hz, 3H), 6.94 (d, J=8.1 Hz, 3H), 6.87 (dd, J=8.1, 2.0 Hz, 3H), 6.44 (t, J=4.7 Hz, 3H), 4.38 (d, J=4.7 Hz, 6H), 4.05 (q, J=7.1 Hz, 6H), 2.81 (q, J=7.6 Hz, 6H), 1.18 (td, J=7.2, 4.0 Hz, 18H).

### Synthesis of GI13

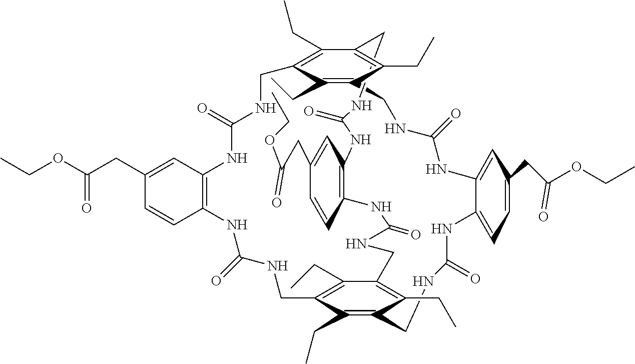

Prepared according to General Procedure E from GI12 (720 mg, 0.791 mmol) in pyridine (150 mL) with DMF (75 mL) and “TEB NCO” (synthesised according to compound 103 of WO 2018/167503, incorporated herein by reference) (311 mg, 0.950 mmol) in toluene (13 mL). The reaction mixture was concentrated to a few mL of DMF and 1 M HCl added to give a precipitate which was redissolved in acetone/water and then evaporated to give the product as a pale brown solid containing 20% water w/w) (1190 mg, 0.769 mmol, 97%).

¹H NMR (400 MHz, DMSO-d₆) δ 8.11-7.88 (m, 6H), 7.84-7.66 (m, 5H), 6.78 (dd, J=8.5, 2.1 Hz, 3H), 6.32 (dt, J=23.7, 5.2 Hz, 5H), 4.29 (s, 12H), 4.07 (q, J=7.1 Hz, 7H), 3.52 (s, 10H), 2.65 (q, J=7.9, 7.2 Hz, 10H), 2.08 (d, J=4.6 Hz, 3H), 1.14 (ddt, J=17.0, 13.1, 8.2 Hz, 28H).

### Synthesis of GI14

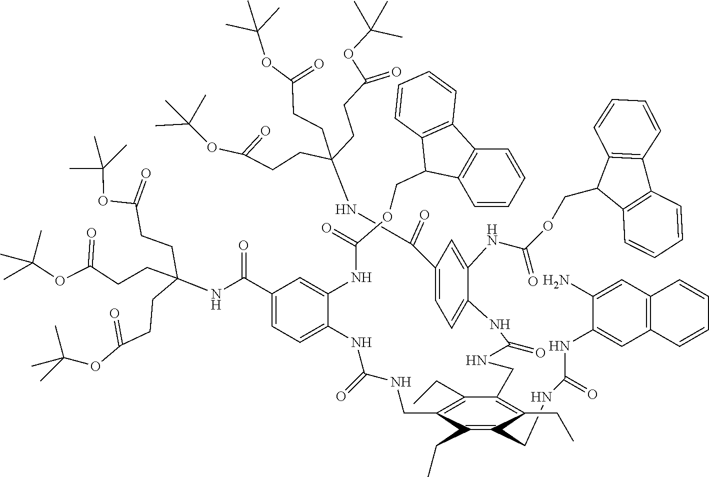

The “G1M-linker” (synthesised according to compound 2 of WO 2018/167503, incorporated herein by reference) (330 mg, 0.428 mmol) and “TEB NCO” (synthesised according to compound 103 of WO 2018/167503, incorporated herein by reference) (70 mg, 0.214 mmol) were dissolved in pyridine (5 mL) and stirred overnight. To this was added a solution of 2,3-diaminonaphthalene (200 mg, 1.26 mmol) in dry pyridine (2 mL). After 5 hours the reaction mixture was evaporated to near dryness and then dry loaded onto RP silica. Purification by RP MPLC (H₂O with increasing acetone). The product was isolated as a white solid (196 mg, 0.097 mmol, 45%).

MS (electrospray, +ve) [M+H]⁺ calculated for C₁₁₆H₁₄₆N₁₁O₂₁ requires: 2029.0689, found: 2029.3

### Synthesis of GI15

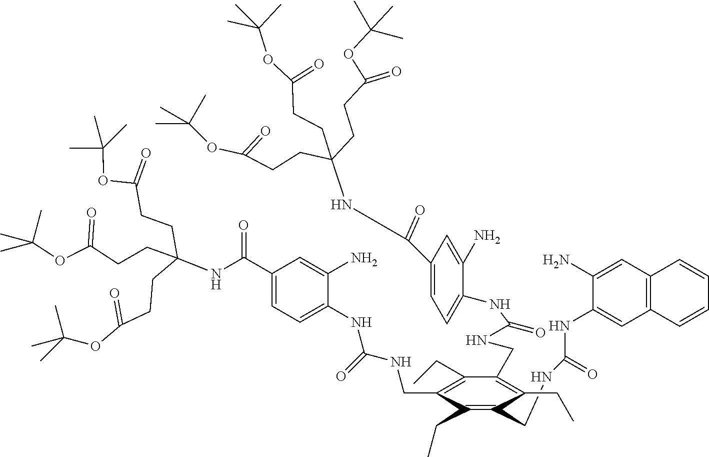

To a solution of GI14 (196 mg, 0.097 mmol) in dry DMF (3 mL) was added piperidine (0.3 mL, 3.0 mmol). After 10 minutes the mixture was diluted with MeCN/water and loaded directly onto a RP MPLC column (H₂O with increasing acetone). Product isolated as a white solid (151 mg, 0.095 mmol, 98%).

¹H NMR (400 MHz, DMSO-d₆) δ 8.08 (s, 1H), 7.58 (dd, J=8.3, 5.2 Hz, 3H), 7.48 (d, J=8.1 Hz, 1H), 7.23-7.14 (m, 3H), 7.14-7.08 (m, 3H), 7.05-7.00 (m, 3H), 4.38 (d, J=11.9 Hz, 6H), 2.82 (s, 6H), 2.16-2.07 (m, 17H), 1.90 (t, J=8.1 Hz, 12H), 1.36 (s, 59H), 1.25-1.13 (m, 10H).

### Synthesis of GI16

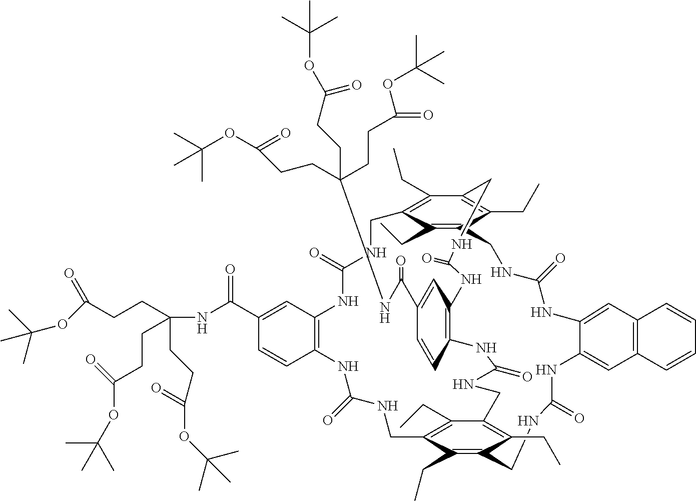

Prepared according to General Procedure E from GI16 (150 mg, 0.095 mmol) in pyridine (20 mL) with DMF (10 mL) and “TEB NCO” (synthesised according to compound 103 of WO 2018/167503, incorporated herein by reference) (37 mg, 0.114 mmol) in DCM (3 mL). The reaction mixture was evaporated to a viscous gum which was dry loaded onto RP silica using acetone and water. Purification by RP MPLC (H₂O with increasing acetone). Product isolated as a white solid (95 mg, 0.050 mmol, 52%)

¹H NMR (400 MHz, DMSO-d₆) δ 8.29 (s, 1H), 8.23 (s, 1H), 8.04 (d, J=1.8 Hz, 2H), 7.94 (d, J=8.5 Hz, 2H), 7.79-7.65 (m, 6H), 7.49 (dd, J=8.4, 2.1 Hz, 2H), 7.43 (s, 2H), 7.37-7.27 (m, 4H), 6.41 (d, J=18.7 Hz, 4H), 6.32 (s, 2H), 4.32 (s, 12H), 2.74 (d, J=46.9 Hz, 13H), 2.14 (t, J=7.9 Hz, 12H), 1.95 (d, J=8.5 Hz, 12H), 1.38 (s, 58H), 1.20-1.09 (m, 18H).

### Synthesis of GI17

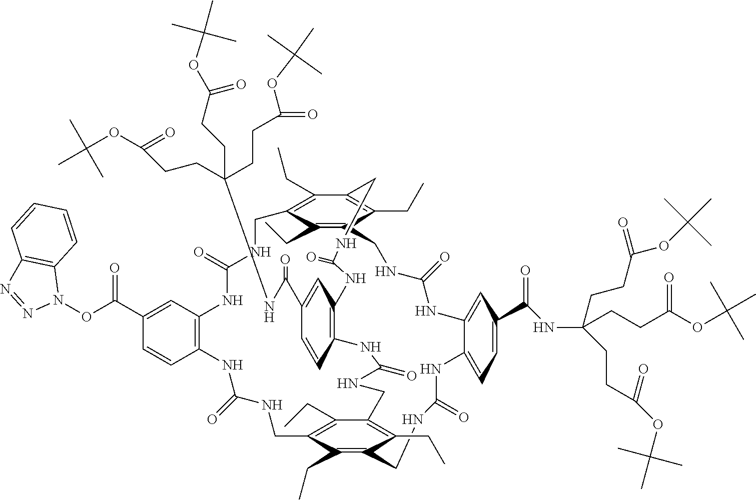

GBM16 (20 mg, 0.016 mmol), HOBt·H₂O (7 mg, 0.049 mmol), HBTU (18 mg, 0.049 mmol) dissolved in DMF (1.6 mL) with DIPEA (9 μL). After stirring for 1 hour, di-tert-butyl 4-amino-4-[2-(tert-butoxycarbonyl)ethyl]heptanedioate (33.7 mg, 0.081 mmol) added as a solution in pyridine (1.6 mL). The reaction was left to stir overnight. Reaction mixture was concentrated under vacuum and then purified using RP MPLC (H₂O with increasing acetone). Mixture of macrocyclic products obtained, major product obtained as a white solid (11 mg, 5.4 μmol, 34%).

¹H NMR (400 MHz, Methanol-d₄) δ 8.08-7.87 (m, 6H), 7.72-7.58 (m, 4H), 7.52 (dt, J=8.9, 2.5 Hz, 3H), 4.43 (d, J=18.3 Hz, 12H), 2.99-2.60 (m, 12H), 2.36-2.20 (m, 12H), 2.08 (t, J=8.0 Hz, 12H), 1.43 (s, 54H), 1.19 (dd, J=10.5, 5.6 Hz, 18H).

MS (electrospray, +ve) [M+2Na]²⁺ calculated for C₁₀₇H₁₄₇N₁₇Na₂O₂₂ requires: 1034.5362, found: 1034.6.

### Synthesis of GI18

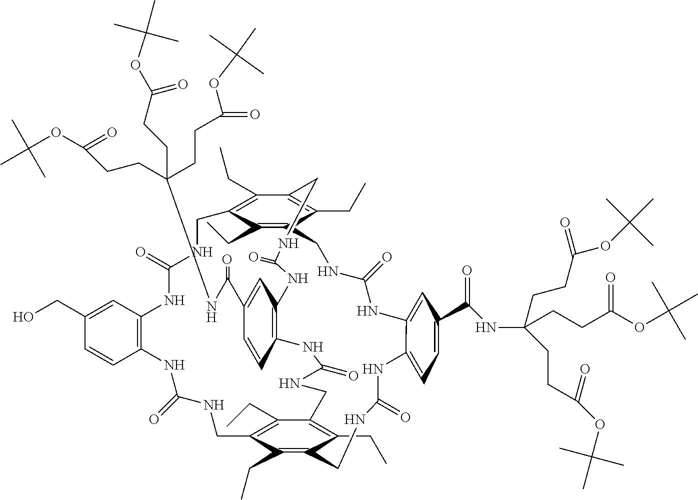

GI17 (142 mg, 0.070 mmol) dissolved in THF (1.4 mL). NaBH₄ (6 mg, 0.175 mmol) added, and the reaction stirred overnight. Remaining borohydride quenched with 1 M HCl and THF removed under vacuum. Purified using RP MPLC (H₂O with increasing acetone). Product obtained as a white solid (77 mg, 0.041 mmol, 58%).

¹H NMR (400 MHz, Methanol-d₄) δ 7.98 (d, J=8.6 Hz, 2H), 7.90 (s, 2H), 7.73 (s, 1H), 7.62 (d, J=14.5 Hz, 2H), 7.55 (d, J=8.8 Hz, 2H), 7.11 (d, J=8.8 Hz, 1H), 4.57 (s, 2H), 4.43 (dd, J=20.3, 12.9 Hz, 12H), 2.97-2.64 (m, 12H), 2.33-2.21 (m, 12H), 2.13-2.01 (m, 12H), 1.45 (s, 54H), 1.25-1.15 (m, 18H).

MS (electrospray, +ve) [M+2Na]²⁺ calculated for C₁₀₁H₁₄₆N₁₄Na₂O₂₁ requires: 969.0302, found: 969.0.

### Synthesis of GI19

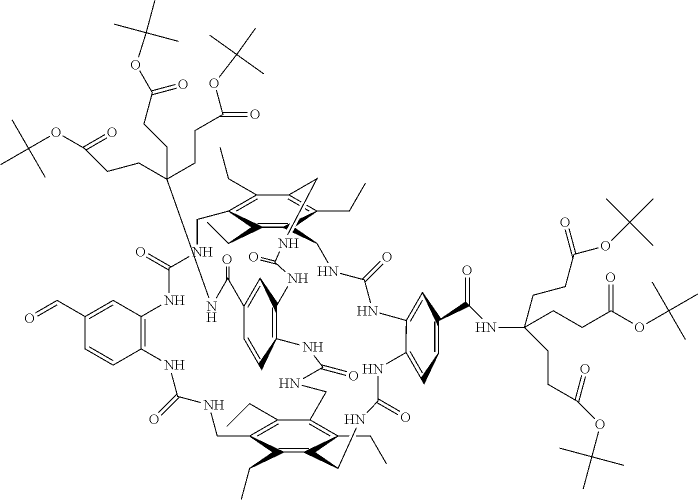

GI18 (167 mg, 0.088 mmol) and Dess-Martin Periodinane (39.3 mg, 0.093 mmol) were dissolved in DCM (0.5 mL). The reaction was stirred overnight. The solvent was evaporated, and the residue quenched with sat. aq. NaHCO₃. The suspension diluted with MeCN and then purified using RP MPLC (H₂O with increasing acetone). Product obtained as a white solid (132 mg, 0.070 mmol, 79%).

¹H NMR (400 MHz, Methanol-d₄) δ 9.87 (s, 1H), 8.16 (d, J=8.5 Hz, 1H), 8.10 (d, J=1.9 Hz, 1H), 7.95 (d, J=8.5 Hz, 2H), 7.89 (d, J=2.1 Hz, 2H), 7.65 (s, 2H), 7.56 (dd, J=8.6, 2.2 Hz, 2H), 4.51-4.41 (m, 12H), 2.96-2.65 (m, 12H), 2.27 (dd, J=9.7, 6.2 Hz, 12H), 2.09 (t, J=8.1 Hz, 12H), 1.45 (s, 54H), 1.26-1.15 (m, 18H).

MS (electrospray, +ve) [M+2Na]²⁺ calculated for C₁₀₁H₁₄₄N₁₄Na₂O₂₁ requires: 968.0224, found: 968.0.

### Synthesis of GI20

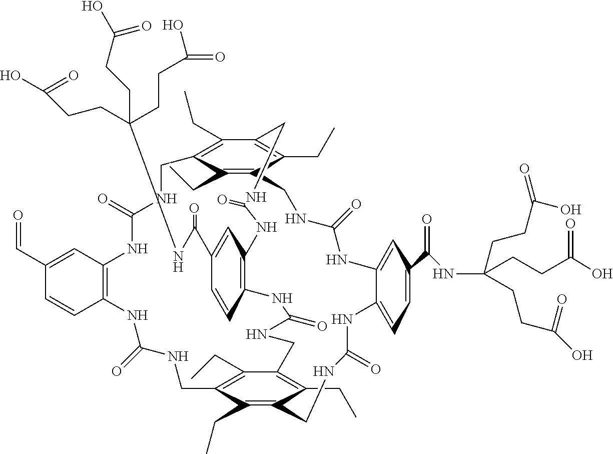

GI19 (132 mg, 0.070 mmol) was dissolved in DCM (3.5 mL) and cooled to 0° C. TFA (0.35 mL) added and the reaction was left stirring in the ice bath as it melted overnight. The product was precipitated with Et₂O and the solid separated by centrifugation. Off white solid (105 mg, 0.068 mmol, 97%).

¹H NMR (400 MHz, Methanol-d₄) δ 7.99-7.50 (m, 9H), 4.49-4.36 (m, 12H), 2.88-2.67 (m, 12H), 2.42-2.26 (m, 12H), 2.16 (s, 12H), 1.34-1.23 (m, 18H).

MS (electrospray, +ve) [M+H]⁺ calculated for C₇₇H₉₇N₁₄O₂₁ requires: 1553.6947, found: 1553.6.

### Synthesis of GI21

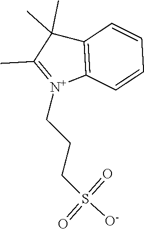 [SMILES: CC1=[N+](CCCS(=O)(=O)[O-])c2ccccc2C1(C)C]

2,3,3-trimethyl-3H-indole (1.00 g, 6.28 mmol) and 1,3-Propanesultone (767 mg, 6.28 mmol) dissolved in toluene (33 mL) and refluxed for 24 hours. Reaction cooled, filtered and precipitate washed with petrol to give the product (430 mg, 1.51 mmol, 24%).

¹H NMR (400 MHz, Methanol-d₄) δ 8.07-7.95 (m, 1H), 7.82-7.73 (m, 1H), 7.73-7.64 (m, 2H), 4.82-4.69 (m, 2H), 3.35 (s, 3H), 3.01 (t, J=6.5 Hz, 2H), 2.47-2.29 (m, 2H), 1.61 (s, 6H).

### Synthesis of GI22

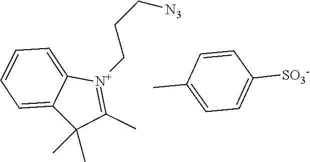

2,3,3-trimethyl-3H-indole (100 mg, 0.628 mmol) was combined with 3-azidopropyl 4-methylbenzenesulfonate (160 mg, 0.628 mmol). The two compounds were stirred in a sealed microwave vial at 70° C. for 2 hours. After this time the yellow liquid had become a dark purple solid. This was taken up in DCM and run through a silica plug. Purple solid obtained, used without further purification.

¹H NMR (400 MHz, Methanol-d₄) δ 7.74-7.69 (m, 2H), 7.27-7.22 (m, 2H), 7.11-7.05 (m, 2H), 6.72 (td, J=7.4, 1.0 Hz, 1H), 6.62 (dd, J=8.2, 0.9 Hz, 1H), 3.62 (t, J=6.8 Hz, 2H), 3.37 (t, J=6.5 Hz, 2H), 2.37 (s, 3H), 1.92-1.87 (m, 2H), 1.61-1.31 (m, 3H), 1.30 (s, 6H).

### Synthesis of GI23

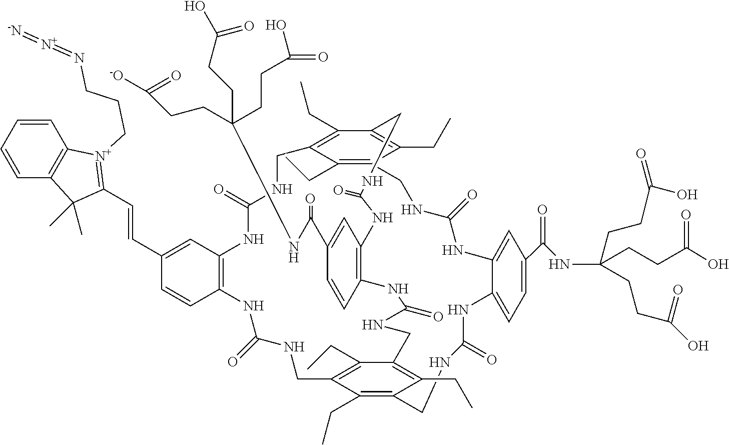

GI22 (3.2 mg, 7.7 μmol), GI20 (6 mg, 3.9 μmol) and ammonium acetate (0.9 mg, 11.6 μmol) were dissolved in DMF (150 μL) and stirred overnight. Solvent was evaporated and the residue purified by RP MPLC (H₂O with increasing MeCN (0.1% formic acid)). Product isolated as an orange solid (1 mg, 0.6 μmol, 15%).

¹H NMR (400 MHz, Deuterium Oxide (PBS) with added DMF) δ 7.74-7.09 (m, 20H), 4.27 (s, 12H), 2.14-2.00 (m, 17H), 1.92 (s, 17H), 1.37-1.34 (m, 2H), 1.01 (s, 18H).

MS (electrospray, +ve) [M+H]⁺ calculated for C₉₁H₁₁₃N₁₈O₂₀ requires: 1777.8373, found: 1777.7.

### Synthesis of GI24

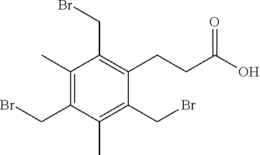 [SMILES: Cc1c(CBr)c(C)c(CBr)c(CCC(=O)O)c1CBr]

3-(3,5-dimethylphenyl)propanoic acid (5.00 g, 28.1 mmol) dissolved in HBr/AcOH (33%, 53 mL). Paraformaldehyde (8.84 g, 295 mmol) and ZnBr₂ (10.1 g, 44.9 mmol) added, and reaction mixture heated to 100° C. for 18 hours. Reaction cooled and precipitate isolated by filtration, washing with AcOH (50 mL) and water (800 mL) (12.2 g, 26.6 mmol, 95%).

¹H NMR (400 MHz, DMSO-d₆) δ 4.75 (d, J=1.5 Hz, 6H), 3.14-2.96 (m, 2H), 2.58 (d, J=18.5 Hz, 2H), 2.40 (d, J=1.1 Hz, 6H).

¹³C NMR (101 MHz, DMSO-d₆) δ 173.44, 140.36, 138.81, 134.06, 132.99, 31.54, 31.22, 24.39, 15.21.

### Synthesis of GI25

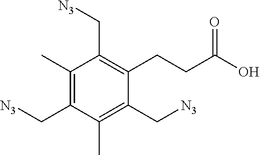 [SMILES: Cc1c(CN=[N+]=[N-])c(C)c(CN=[N+]=[N-])c(CCC(=O)O)c1CN=[N+]=[N-]]

GI24 (14.0 g, 30.6 mmol) dissolved in DMF (12.0 mL) and NaN₃ (11.9 g, 184 mmol) added in 4 portions over 20 minutes. Reaction mixture left to stir for 40 hours then quenched with 1 M HCl (400 mL). Resultant suspension extracted with EtOAc (3×1.5 L) and the combined organics washed with water (5×400 mL), brine (400 mL) and dried (Na₂SO₄). Solvents evaporated under reduced pressure to an oil which crystallises to give the product (9.89 g, 28.8 mmol, 94%).

¹H NMR (400 MHz, Chloroform-d) δ 4.54 (s, 4H), 4.51 (s, 2H), 3.25-3.10 (m, 2H), 2.72-2.57 (m, 2H), 2.48 (d, J=0.7 Hz, 6H).

¹³C NMR (101 MHz, Chloroform-d) δ 177.51, 140.23, 139.05, 132.37, 130.94, 49.04, 48.79, 35.37, 25.18, 16.73.

### Synthesis of GI26

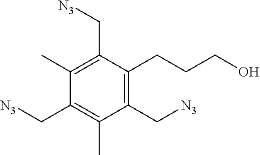 [SMILES: Cc1c(CN=[N+]=[N-])c(C)c(CN=[N+]=[N-])c(CCCO)c1CN=[N+]=[N-]]

GI25 (9.89 g, 28.8 mmol) dissolved in THF (30 mL) and 1 M BH₃.THF in THF (58 mL, 58.0 mmol) added dropwise. After 5 hours, further 1 M BH₃.THF in THF (20 mL, 20.0 mmol) added and the reaction left for 16 hours. Quenched with sat. aq. NaHCO₃ (60 mL). THF blown off under a stream of N₂ and the remnants extracted with EtOAc (100 mL). Organic layer dried (Na₂SO₄) and concentrated under reduced pressure. Crude product purified by MPLC (petrol with increasing EtOAc) to give the product as white crystals (4.42 g, 13.4 mmol, 47%).

¹H NMR (400 MHz, Chloroform-d) δ 4.53 (s, 4H), 4.50 (s, 2H), 3.77 (t, J=5.9 Hz, 2H), 2.99-2.88 (m, 2H), 2.46 (s, 6H), 1.86-1.74 (m, 2H).

### Synthesis of GI27

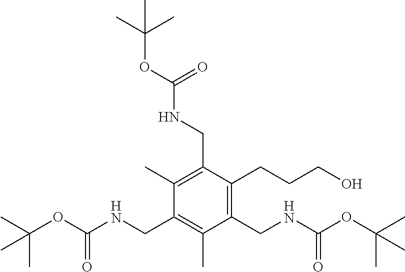 [SMILES: Cc1c(CNC(=O)OC(C)(C)C)c(C)c(CNC(=O)OC(C)(C)C)c(CCCO)c1CNC(=O)OC(C)(C)C]

GI26 (1.40 g, 4.25 mmol), Boc₂O (4.18 g, 19.1 mmol) dissolved in THF (80 mL). Slurry of Pd/C (200 mg, 10% w/w) added, followed by Et₃N (1.80 mL, 12.8 mmol). Reaction placed under hydrogen atmosphere and stirred overnight. Reaction mixture centrifuged and the supernatant concentrated under reduced pressure, before being redissolved in EtOAc (100 mL) and washed with 5% aq. KHSO₄ (100 mL), sat. aq. NaHCO₃ (100 mL) and brine (100 mL) then dried (Na₂SO₄). Organic solvents removed in vacuo and the crude product purified by MPLC (petrol with increasing EtOAc) to give the product as a white solid (2.02 g, 3.64 mmol, 86%).

¹H NMR (400 MHz, Chloroform-d) δ 4.37 (d, J=11.0 Hz, 7H), 3.72 (t, J=5.7 Hz, 2H), 2.94-2.77 (m, 2H), 2.36 (s, 6H), 1.76-1.65 (m, 2H), 1.44 (d, J=2.4 Hz, 27H).

MS (electrospray, +ve) [2M−Boc+H]⁺ calculated for C₅₃H₉₁N₆O₁₂ requires: 1003.6690, found: 1003.6.

### Synthesis of GI28

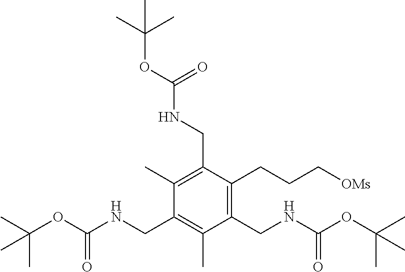 [SMILES: Cc1c(CNC(=O)OC(C)(C)C)c(C)c(CNC(=O)OC(C)(C)C)c(CCCOS(C)(=O)=O)c1CNC(=O)OC(C)(C)C]

GI27 (4.41 g, 7.99 mmol) dissolved in DCM (16 mL). Et₃N (2.80 mL, 20.0 mmol) added and the reaction cooled to 0° C., followed by addition of MsCl (0.62 mL, 8.00 mmol) over a period of 10 minutes. The reaction left to stir at RT for 3 h, cooled to 0° C. and MsCl (0.40 ml, 5.17 mmol) added. After 3 h, reaction mixture diluted with DCM (60 mL) and partitioned with brine (60 mL). Organic dried (Na₂SO₄) and concentrated under reduced pressure. Crude product purified by MPLC (petrol with increasing EtOAc) to give the product as a white solid (4.85 g, 7.70 mmol, 96%).

¹H NMR (400 MHz, Chloroform-d) δ 4.38-4.29 (m, 9H), 3.11 (s, 3H), 2.99-2.78 (m, 2H), 2.36 (s, 6H), 1.89 (dq, J=11.8, 5.9 Hz, 2H), 1.43 (s, 27H).

MS (electrospray, +ve) [M+Na]⁺ calculated for C₃₀H₅₁N₃NaO₉S requires: 652.3239, found: 652.3.

### Synthesis of GI29

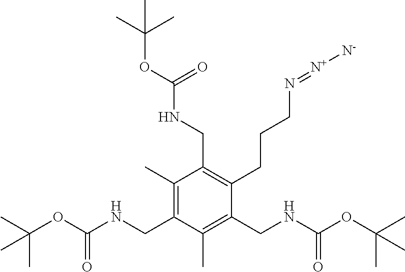 [SMILES: Cc1c(CNC(=O)OC(C)(C)C)c(C)c(CNC(=O)OC(C)(C)C)c(CCCN=[NH+][NH-])c1CNC(=O)OC(C)(C)C]

GI27 (1.31 g, 2.08 mmol) dissolved in DMF (21 mL), NaN₃ (676 mg, 10.4 mmol) added and the reaction stirred at RT for 22 hours. The reaction mixture was diluted with water (100 mL) and extracted with Et₂O (5×50 mL). Organic layer dried (MgSO₄) and concentrated under reduced pressure. Crude oil was purified by RP MPLC (water with increasing acetone) to give the product as a white solid (800 mg, 1.39 mmol, 67%).

¹H NMR (400 MHz, Chloroform-d) δ 4.50-4.19 (m, 9H), 3.43 (t, J=6.7 Hz, 2H), 2.86-2.72 (m, 2H), 2.37 (s, 6H), 1.79-1.68 (m, 2H), 1.45 (s, 27H).

MS (electrospray, +ve) [2M−Boc+H]⁺ calculated for C₅₃H₈₉N₁₂O₁₀ requires: 1053.6820, found: 1053.6.

### Synthesis of GI30

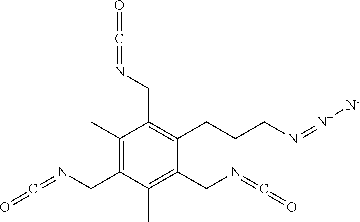 [SMILES: Cc1c(CN=C=O)c(C)c(CN=C=O)c(CCCN=[NH+][NH-])c1CN=C=O]

Prepared according to General Procedure G from GI29 (350 mg, 0.617 mmol), 2-CI-Pyr (0.51 mL, 5.46 mmol) and Tf₂O (0.46 mL, 2.73 mmol) in DCM (6 mL). Product obtained as white crystals (170 mg, 0.480 mmol, 79%).

¹H NMR (400 MHz, Chloroform-d) δ 4.50 (s, 6H), 3.49 (t, J=6.2 Hz, 2H), 2.92-2.76 (m, 2H), 2.48 (s, 6H), 1.79 (dq, J=11.9, 6.1 Hz, 2H).

### Synthesis of GI31

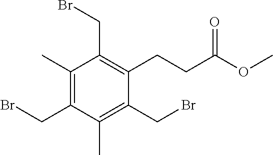 [SMILES: COC(=O)CCc1c(CBr)c(C)c(CBr)c(C)c1CBr]

GI24 (11.7 g, 25.5 mmol) dissolved in MeOH (60 mL) at 0° C. and SOCl₂ (0.37 mL, 5.01 mmol) added dropwise. Stirred at RT for 16 hours then diluted with DCM (200 mL). Organic washed with sat. aq. NaHCO₃ (200 mL) then concentrated under reduced pressure to give the product as a white solid (11.5 g, 24.4 mmol, 96%).

¹H NMR (400 MHz, Chloroform-d) δ 4.59 (s, 4H), 4.57 (s, 2H), 3.76 (s, 3H), 3.30-3.15 (m, 2H), 2.72 (s, 2H), 2.47 (s, 6H).

### Synthesis of GI32

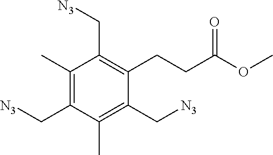 [SMILES: COC(=O)CCc1c(CN=[N+]=[N-])c(C)c(CN=[N+]=[N-])c(C)c1CN=[N+]=[N-]]

GI31 (10.6 g, 22.5 mmol) dissolved in DMF (225 mL) and NaN₃ (8.79 g, 135 mmol) added in four portions over 20 minutes. Stirred at RT for 16 hours then diluted with water (600 mL). Extracted with EtOAc (3×300 mL) and the combined organics washed with water (5×400 mL) and brine (400 mL) then dried (Na₂SO₄). Crude product isolated after removal of solvent under reduced pressure, then purified by MPLC (petrol with increasing EtOAc) to give the product as white crystals (7.25 g, 20.3 mmol, 90%).

¹H NMR (400 MHz, Chloroform-d) δ 4.51 (d, J=8.2 Hz, 6H), 3.74 (s, 3H), 3.20-3.08 (m, 2H), 2.60-2.51 (m, 2H), 2.47 (s, 6H).

¹³C NMR (101 MHz, Chloroform-d) δ 172.69, 139.00, 132.25, 130.91, 52.13, 49.04, 48.85-48.67 (m), 25.47, 16.71.

### Synthesis of GI33

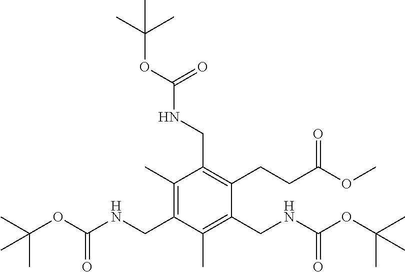 [SMILES: COC(=O)CCc1c(CNC(=O)OC(C)(C)C)c(C)c(CNC(=O)OC(C)(C)C)c(C)c1CNC(=O)OC(C)(C)C]

GI32 (2.00 g, 5.60 mmol), Boc₂O (8.55 g, 39.2 mmol) dissolved in THF (280 mL). Slurry of Pd/C (270 mg) added, followed by Et₃N (2.3 mL, 16.8 mmol). Reaction placed under hydrogen atmosphere and stirred for 64 hours. Reaction mixture centrifuged and the supernatant concentrated under reduced pressure, before being redissolved in EtOAc (150 mL) and washed with 5% aq. KHSO₄ (100 mL), sat. aq. NaHCO₃ (100 mL) and brine (100 mL) then dried (Na₂SO₄). Organic solvents removed in vacuo and the crude product purified by MPLC (petrol with increasing EtOAc) to give the product as a white solid (4.19 g, 4.19 mmol, 75%).

¹H NMR (400 MHz, Chloroform-d) δ 4.43 (d, J=6.4 Hz, 2H), 4.39-4.30 (m, 7H), 3.69 (s, 3H), 3.12-3.01 (m, 2H), 2.55-2.44 (m, 2H), 2.36 (s, 6H), 1.51-1.39 (m, 27H).

MS (electrospray, +ve) [M+Na]⁺ calculated for C₃₀H₄₉N₃NaO₃ requires: 602.3412, found: 602.3

### Synthesis of GI34

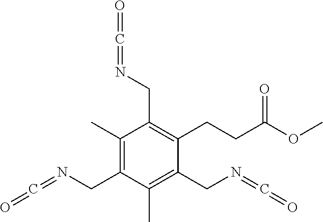 [SMILES: COC(=O)CCc1c(CN=C=O)c(C)c(CN=C=O)c(C)c1CN=C=O]

Prepared according to General Procedure G from GI33 (153 mg, 0.264 mmol), 2-CI-Pyr (0.22 mL, 2.38 mmol) and Tf₂0 (0.20 mL, 1.19 mmol) in DCM (9 mL). Product obtained as white crystals (88 mg, 0.246 mmol, 93%).

¹H NMR (400 MHz, Chloroform-d) δ 4.52 (s, 4H), 4.50 (s, 2H), 3.74 (s, 3H), 3.18-3.09 (m, 2H), 2.59-2.52 (m, 2H), 2.48 (s, 6H).

### Synthesis of GI35

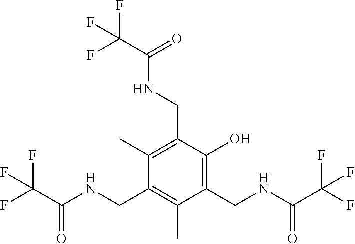 [SMILES: Cc1c(CNC(=O)C(F)(F)F)c(C)c(CNC(=O)C(F)(F)F)c(O)c1CNC(=O)C(F)(F)F]

3,5-dimethylphenol (112 mg, 0.917 mmol) and 2,2,2-trifluoro-N-hydroxymethyl)acetamide (517 mg, 3.613 mmol) dissolved in DCM (3 mL). TFA (1 mL) added, followed by 1 mM BF₃·OEt₂ in Et₂O (3.21 mL, 3.21 mmol). The reaction was stirred for 18 h, then poured onto ice. Diluted with EtOAc (200 mL), washed with water (3×100 mL), dried (Na₂SO₄) and concentrated to an orange foam. This was purified by MPLC (DCM with increasing EtOAc) to give the product as a white solid (216 mg, 0.434 mmol, 47%).

¹H NMR (400 MHz, Methanol-d₄) δ 4.81 (s, 1H), 4.56 (s, 4H), 4.54 (s, 2H), 2.36 (s, 6H).

### Synthesis of GI36

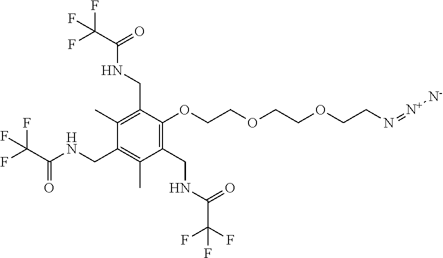 [SMILES: Cc1c(CNC(=O)C(F)(F)F)c(C)c(CNC(=O)C(F)(F)F)c(OCCOCCOCCN=[NH+][NH-])c1CNC(=O)C(F)(F)F]

GI35 (2.00 g, 4.02 mmol), K₂CO₃ (1.22 g, 8.85 mmol), NaI (0.600 g, 4.02 mmol) and azido-PEG₃ Ts (1.66 g, 5.03 mmol) dissolved in DMF (10 mL) and the reaction stirred at 70° C. for 16 hours. Solvent partially removed and partitioned between EtOAc and water. The organic layer was washed with more water, then brine, dried (Na₂SO₄) and concentrated to give a brown oil. Purification by MPLC (DCM with increasing EtOAc) gave the product as a gum (2.35 g, 3.59 mmol, 89%).

¹H NMR (400 MHz, Chloroform-d) δ 7.39 (d, J=6.1 Hz, 2H), 6.28 (s, 1H), 4.59 (t, J=4.8 Hz, 6H), 4.03-3.99 (m, 2H), 3.93-3.89 (m, 2H), 3.81-3.77 (m, 2H), 3.71-3.67 (m, 2H), 3.65 (dd, J=5.5, 4.4 Hz, 2H), 3.38 (dd, J=5.5, 4.3 Hz, 2H), 2.43 (s, 6H).

### Synthesis of GI37

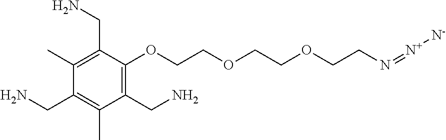 [SMILES: Cc1c(CN)c(C)c(CN)c(OCCOCCOCCN=[NH+][NH-])c1CN]

GI36 (2.35 g, 3.59 mmol) dissolved in MeOH (30 mL) and aq. conc. NH₃ (20 mL) added. After stirring for 70 h, solvents and ammonia were evaporated, redissolving in water and repeating evaporation to remove all NH₃. Product isolated as a crude oil (1.32 g, 3.59 mmol).

### Synthesis of GI38

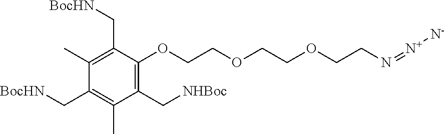 [SMILES: Cc1c(CNC(=O)OC(C)(C)C)c(C)c(CNC(=O)OC(C)(C)C)c(OCCOCCOCCN=[NH+][NH-])c1CNC(=O)OC(C)(C)C]

GI37 (1.32 g, 3.59 mmol) dissolved in THF (36 mL). Boc₂O(3.53 g, 16.2 mmol) was added, followed by K₂CO₃ (1.51 g, 10.9 mmol) and the reaction stirred for 30 m. Et₂O (50 mL) was added and stirred for 10 m. The suspension was filtered, and the filtrate concentrated and purified by MPLC (DCM with increasing Et₂O) to give the product as a white solid (1.10 g, 1.65 mmol, 46%).

¹H NMR (400 MHz, Chloroform-d) δ 4.97 (s, 3H), 4.42-4.34 (m, 6H), 3.94 (dd, J=5.5, 3.1 Hz, 2H), 3.85 (dd, J=5.7, 2.9 Hz, 2H), 3.75-3.68 (m, 6H), 3.41-3.38 (m, 2H), 2.39 (s, 6H), 1.44 (d, J=4.2 Hz, 27H).

### Synthesis of GI39

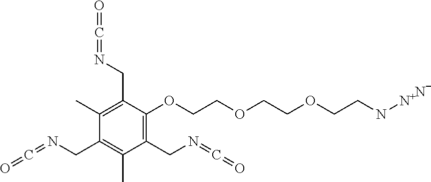

GI38 (134 mg, 0.201 mmol) dissolved in DCM (2.5 mL) with 2-CI-Pyr (0.17 mL, 1.809 mmol), Tf₂O (0.15 mL, 0.904 mmol) was added dropwise, and the reaction stirred for 30 minutes. The reaction mixture was passed through a plug of silica and the solvent evaporated to give the product as a clear oil (89 mg, 0.201 mmol, 100%).

¹H NMR (400 MHz, Chloroform-d) δ 4.59 (s, 4H), 4.47 (s, 2H), 4.04-4.01 (m, 2H), 3.90-3.87 (m, 2H), 3.78-3.71 (m, 6H), 3.44-3.40 (m, 2H), 2.45 (s, 6H).

### Synthesis of GI40

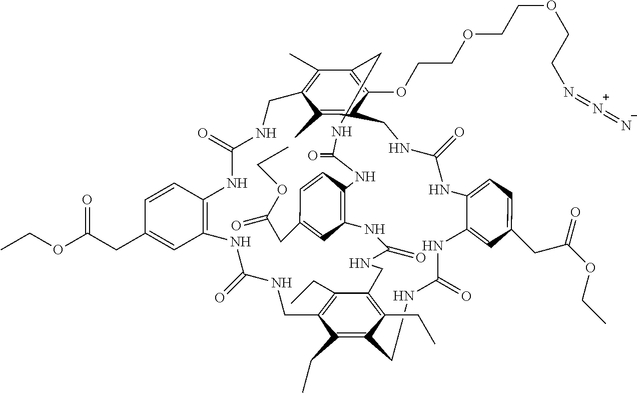

Prepared according to General Procedure E from GI12 (786 mg, 0.864 mmol) and GI39 (461 mg, 1.04 mmol) in pyridine (115 mL), DMF (60 mL) and toluene (14 mL). Product dry-loaded onto C₁₈ silica and purified by RP MPLC (H₂O with increasing acetone) to give the product as a brown solid (228 mg, 0.168 mmol, 20%).

¹H NMR (400 MHz, DMSO-d₆) δ 7.54 (dt, J=21.9, 7.1 Hz, 6H), 6.93-6.76 (m, 3H), 4.30 (d, J=18.4 Hz, 12H), 4.14-4.02 (m, 6H), 3.87 (s, 2H), 3.71 (s, 2H), 3.66-3.50 (m, 12H), 2.74 (s, 6H), 2.36 (s, 6H), 1.24-1.12 (m, 18H)

### Synthesis of GI41

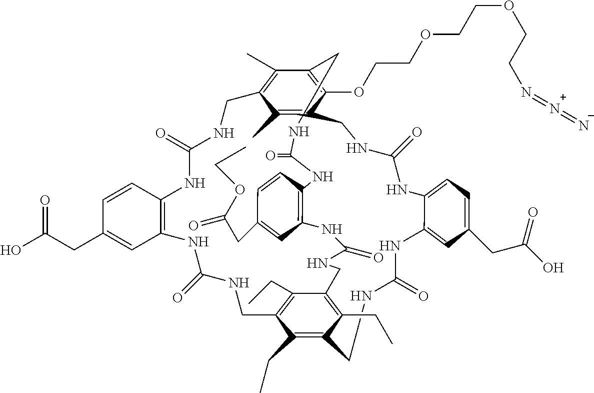

Prepared according to General Procedure E from GI40 (228 mg, 0.168 mmol) and NaOH (54 mg, 1.35 mmol) in EtOH (8 mL) and water (8 mL). Purified by HPLC to give product as a white solid (96 mg, 0.076 mmol, 45%).

¹H NMR (400 MHz, Deuterium Oxide with Na₂CO₃) δ 7.46-7.13 (m, 6H), 6.83 (s, 3H), 4.25 (d, J=31.7 Hz, 12H), 3.80 (d, J=8.5 Hz, 2H), 3.64 (s, 2H), 3.54-3.20 (m, 12H), 3.13 (d, J=2.9 Hz, 2H), 2.62 (s, 6H), 2.12 (d, J=37.5 Hz, 6H), 1.03 (d, J=9.3 Hz, 9H).

MS (electrospray, +ve) [M+H]⁺ calculated for C₆₂H₇₆N₁₅O₁₅ requires: 1270.5640, found: 1270.5.

### Synthesis of GI42

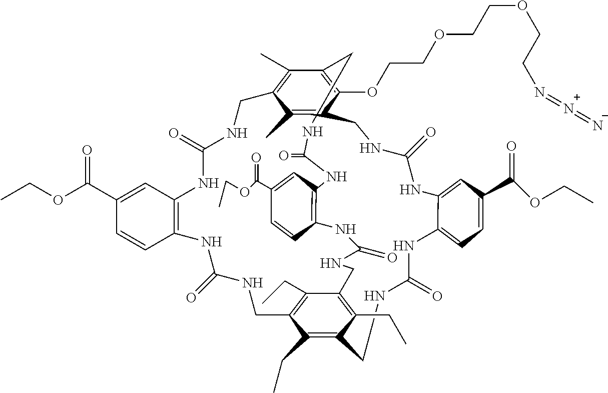

Prepared according to General Procedure E from “3-EtHR” (synthesised according to compound 25c of WO 2020/058322, incorporated herein by reference) (130 mg, 0.150 mmol) and GI39 (84 mg, 0.190 mmol) in pyridine (20 mL), DMF (10 mL) and toluene (2.5 mL). Impure product isolated as an off-white precipitate (198 mg, 0.150 mmol, 100%).

¹H NMR (400 MHz, DMSO-d₆) δ 8.18-8.02 (m, 4H), 7.84-7.70 (m, 2H), 7.68-7.51 (m, 3H), 7.39-7.16 (m, 1H), 6.67-6.35 (m, 3H), 4.81 (s, 1H), 4.50-4.09 (m, 15H), 3.65-3.48 (m, 5H), 2.83-2.60 (m, 6H), 2.40 (s, 5H), 1.35-1.24 (m, 9H), 1.15 (q, J=9.2, 7.5 Hz, 9H).

### Synthesis of GI43

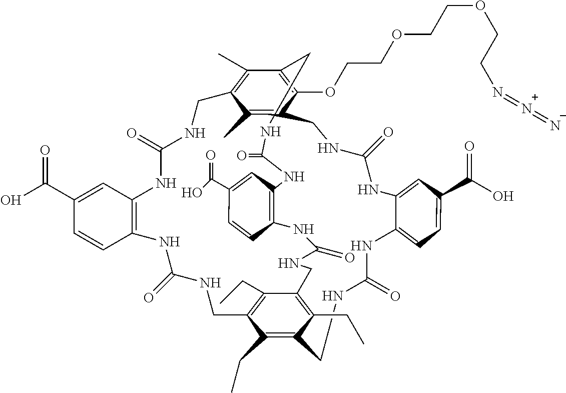

Prepared according to General Procedure E from GI42 (197 mg, 0.150 mmol) and NaOH (54 mg, 1.35 mmol) in EtOH (7 mL) and water (7 mL). Purified by HPLC to give product as a white solid (50 mg, 0.041 mmol, 27%).

¹H NMR (400 MHz, Deuterium Oxide (with scyllo-inosito+Na₂CO₃)) δ 7.96 (d, J=2.7 Hz, 3H), 7.60 (d, J=8.5 Hz, 3H), 7.45 (d, J=8.5 Hz, 3H), 4.05 (s, 4H), 3.96 (s, 2H), 3.76 (s, 2H), 3.59 (s, 6H), 3.38 (s, 4H), 2.54 (s, 6H), 2.35-2.00 (m, 12H), 1.10 (t, J=7.2 Hz, 9H).

MS (electrospray, +ve) [M+H]⁺ calculated for C₅₉H₇₀N₁₅O₁₅ requires: 1228.5171, found: 1228.4.

### Synthesis of GI44

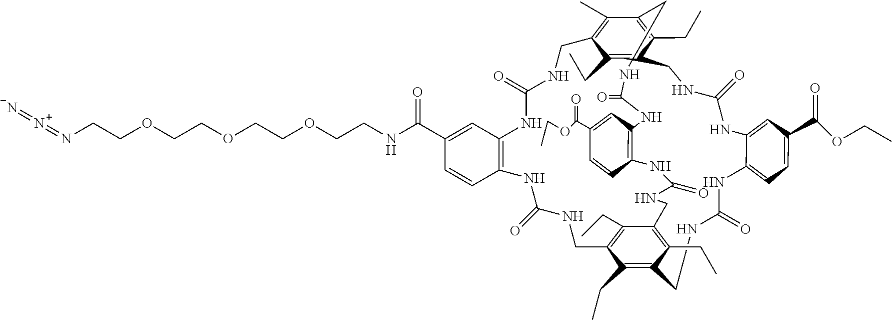

Prepared according to General Procedure A from “2-Et G0_TEB_TEB” (synthesised according to compound 25e of WO 2020/058322, incorporated herein by reference) (20 mg, 0.017 mmol), azido-PEG₃-amine (9 mg, 0.039 mmol), HBTU (16 mg, 0.043 mmol), HOBt·H₂O (8 mg, 0.051 mmol) and DIPEA (9 μL, 0.051 mmol) in THF (1 mL). Purified by RP MPLC (H₂O with increasing MeCN (0.1% formic acid)). Product obtained as a white solid (17 mg, 0.012 mmol, 73%).

¹H NMR (400 MHz, DMSO-d₆) δ 8.48-8.31 (m, 5H), 8.27 (s, 5H), 8.16 (d, J=15.5 Hz, 3H), 8.10 (d, J=8.7 Hz, 2H), 7.98 (d, J=10.2 Hz, 2H), 7.54 (dd, J=8.7, 2.0 Hz, 2H), 7.43 (d, J=7.5 Hz, 1H), 6.84 (s, 3H), 6.63 (s, 3H), 4.27 (q, J=6.9 Hz, 16H), 3.54 (s, 16H), 2.72 (s, 12H), 1.31 (t, J=7.1 Hz, 6H), 1.13 (d, J=7.9 Hz, 18H).

MS (electrospray, +ve), [M+H]²⁺ calculated for C₆₉H₉₁N₁₆O₁₄ requires: 1367.6896, found: 1367.6.

### Synthesis of GI45

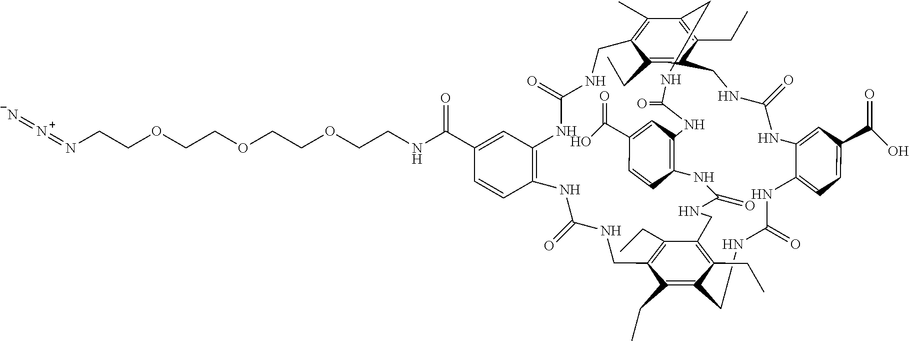

Prepared according to General Procedure E from GI44 (17 mg, 0.012 mmol) and NaOH (15 mg, 0.375 mmol) in EtOH (0.4 mL) and water (0.4 mL). Product obtained as a white precipitate (12 mg, 0.009 mmol, 74%).

¹H NMR (400 MHz, DMSO-d₆) δ 8.41-8.34 (m, 3H), 8.31-8.14 (m, 3H), 8.17-8.06 (m, 4H), 8.06-7.88 (m, 2H), 7.52 (dd, J=8.6, 2.1 Hz, 2H), 7.43 (dd, J=8.8, 2.1 Hz, 1H), 6.50 (d, J=21.4 Hz, 3H), 6.29 (s, 2H), 4.32 (s, 12H), 3.54 (s, 16H), 2.62 (d, J=6.4 Hz, 12H), 1.15 (t, J=7.5 Hz, 18H).

MS (electrospray, +ve) [M+H]⁺ calculated for C₆₅H₈₃N₁₆O₁₄ requires: 1311.6270, found: 1311.5.

### Synthesis of GI46

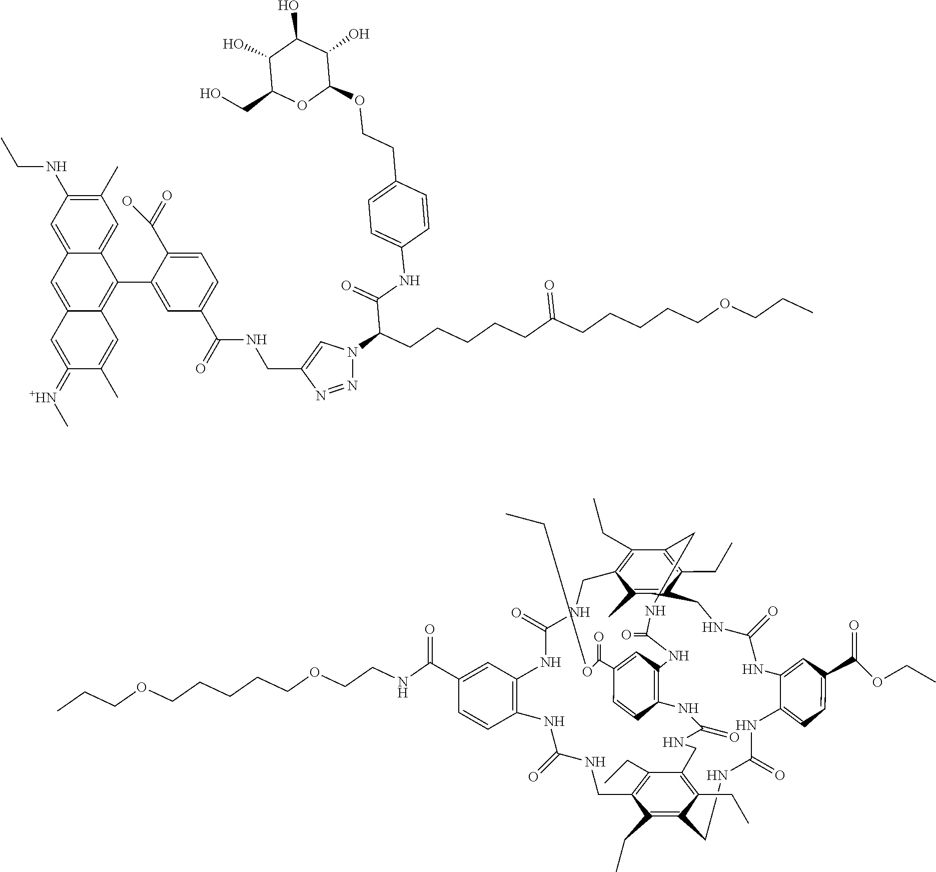

Prepared according to General Procedure A from “2-Et_G0_TEB_TEB” (synthesised according to compound 25e of WO 2020/058322, incorporated herein by reference) (4.0 mg, 0.003 mmol), I22 (5.3 mg, 0.004 mmol), HBTU (2.6 mg, 0.007 mmol), HOBt·H₂O (1.0 mg, 0.007 mmol) and DIPEA (1 μL, 0.007 mmol) in THF (1 mL) with a drop of water. Purified by RP MPLC (THF) to give the product as a red solid (8 mg, 0.003 mmol, 100%).

MS (electrospray, +ve), [M+2H]²⁺ calculated for C₁₂₆H₁₆₃N₂₁O₂ requires: 1217.5974, found: 1217.6

### Synthesis of GI47

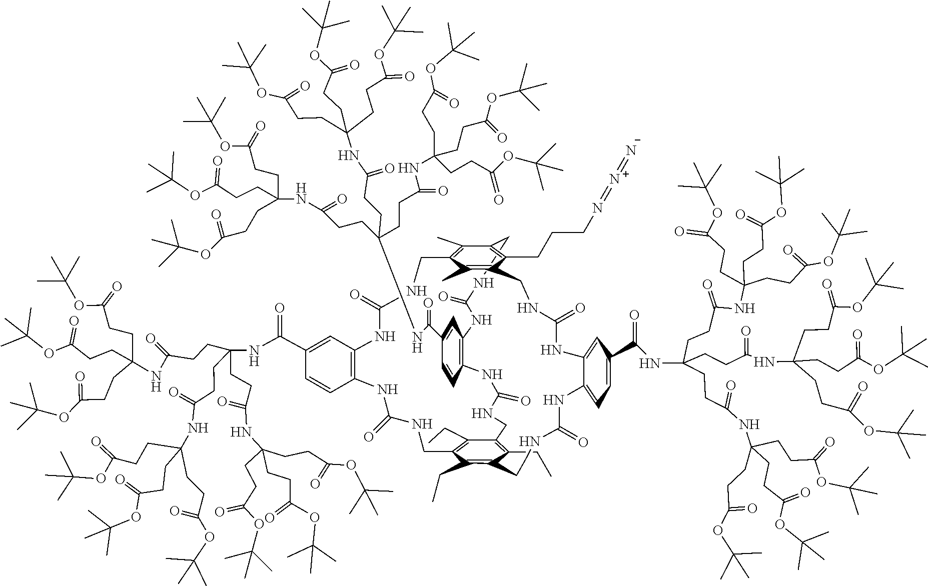

Prepared according to General Procedure E from “3-G2_TEB_HR” (synthesised according to compound 108 of WO 2018/167503, incorporated herein by reference) (360 mg, 0.071 mmol) and GI30 (30 mg, 0.085 mmol) in pyridine (20 mL), DMF (10 mL) and toluene (3 mL). Purified by RP MPLC (H₂O with increasing acetone) to give the product as a white solid (261 mg, 0.048 mmol, 68%).

¹H NMR (400 MHz, Methanol-d₄) δ 8.12 (s, 3H), 8.06-7.88 (m, 6H), 7.69 (d, J=8.6 Hz, 3H), 7.44 (s, 9H) 4.55-4.25 (m, 12H), 3.47 (d, J=6.4 Hz, 2H), 3.04-2.70 (m, 8H), 2.49 (s, 6H), 2.32-2.05 (m, 90H), 1.95 (t, J=8.1 Hz, 54H), 1.75 (s, 2H), 1.43 (s, 243H), 1.31-1.09 (m, 9H).

### Synthesis of GI48

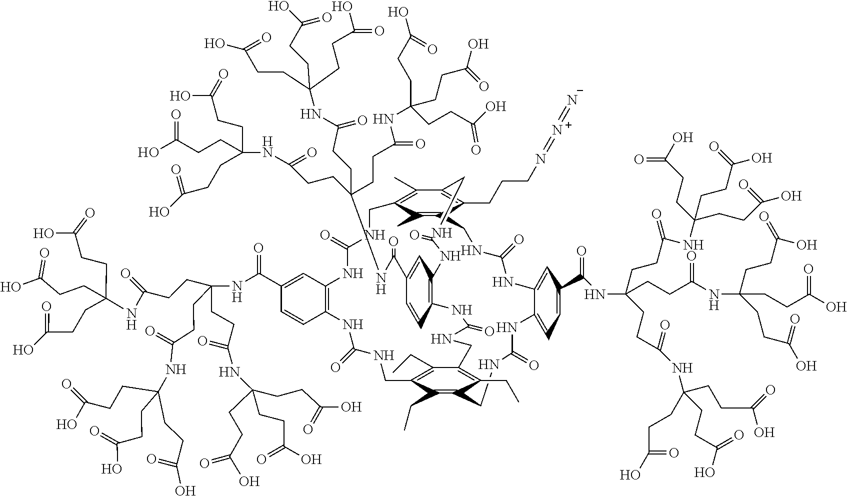

GI47 (75 mg, 0.014 mmol) dissolved in formic acid (0.5 mL) for 4 hours. Formic acid removed under a stream of N₂, residue redissolved in DCM and evaporated again. Solvent traces removed under high vacuum to give the product as a white solid (54 mg, 0.014 mmol, 100%).

¹H NMR (400 MHz, Deuterium Oxide (PBS)) δ 7.76 (d, J=30.4 Hz, 6H), 7.44 (d, J=8.6 Hz, 3H), 4.32 (s, 12H), 3.25 (s, 2H), 2.88 (s, 3H), 2.81-2.67 (m, 5H), 2.66-2.44 (m, 8H), 2.38-1.57 (m, 144H), 1.13-0.93 (m, 9H).

MS (electrospray, +ve), [M+3H]³⁺ calculated for C₁₇₆H₂₄₆N₂₇O₇₂ requires: 1296.8812, found: 1297.0.

### Synthesis of GI49

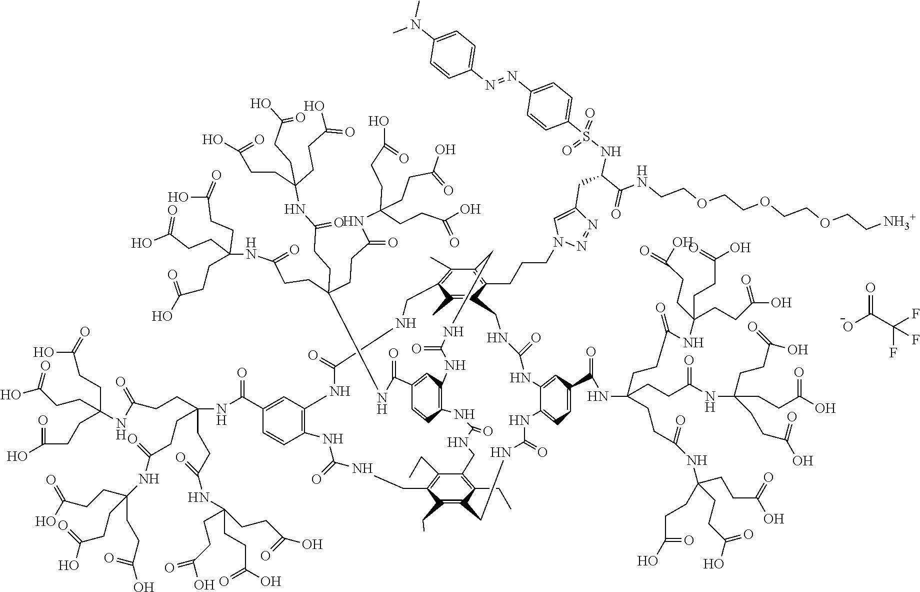

GBM7 (9 mg, 2.00 μmol) dissolved in DCM (0.5 mL) and TFA added (0.25 mL). Stirred for 3 hours then solvents removed under a stream of N₂. Dry loaded onto C₁₈ silica and purified by RP MPLC (H₂O with increasing MeCN (0.1% formic acid)) to give the product as a brown solid (6 mg, 1.30 μmol, 67%).

MS (electrospray, +ve), [M+3H]³⁺ calculated for C₂₀₃H₂₈₄N₃₃O₇₈S requires: 1488.6348, found: 1488.5.

### Synthesis of GI50

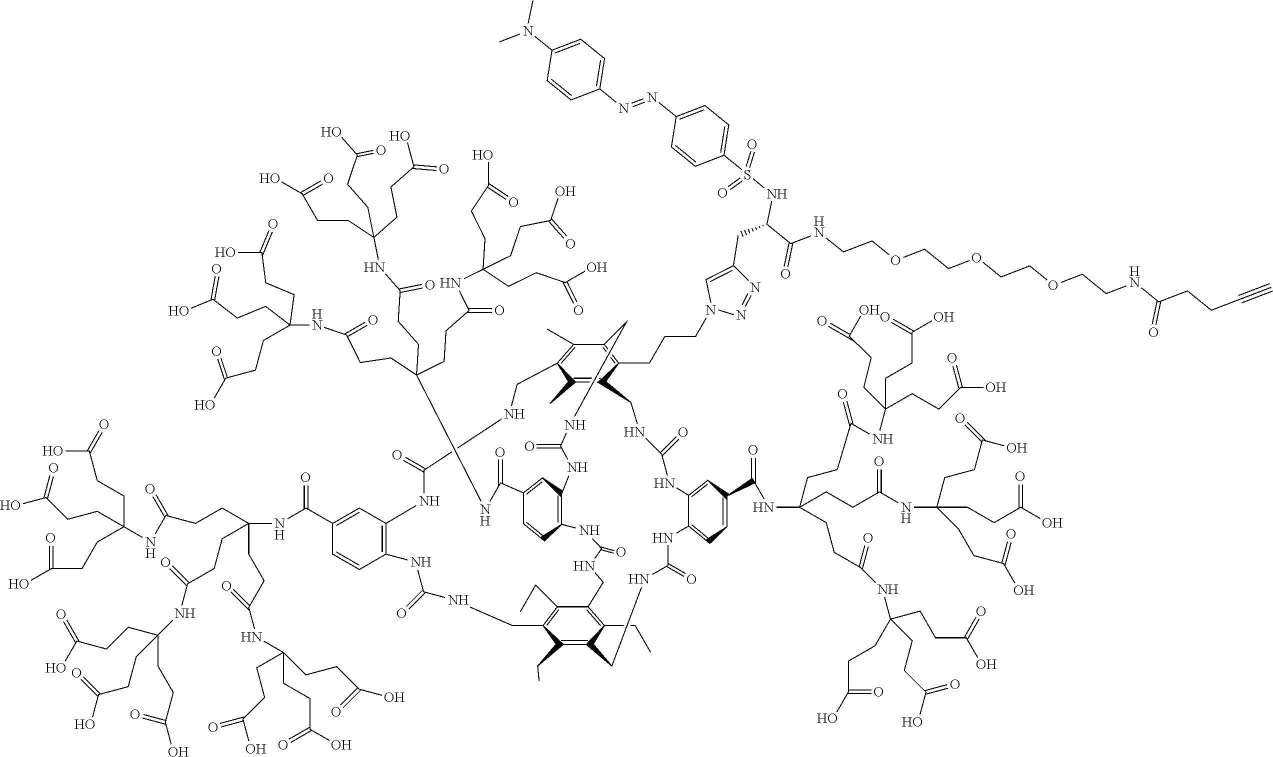

GI49 (3 mg, 0.66 μmol) and NaHCO₃ (2 mg, 26 μmol) dissolved in MeCN (0.5 mL) and water (0.5 mL). Pentynoic acid N-hydroxysuccinimide ester (2 mg, 10 μmol) added, and reaction stirred for 2 hours. Purified by RP MPLC (H₂O with increasing MeCN (0.1% formic acid)) to give the product as a brown solid (3 mg, 0.66 μmol, 100%).

MS (electrospray, +ve), [M+3H]³⁺ calculated for C₂₀₈H₂₈₈N₃₃O₇₉S requires: 1515.3102, found: 1515.4.

### Synthesis of GI51

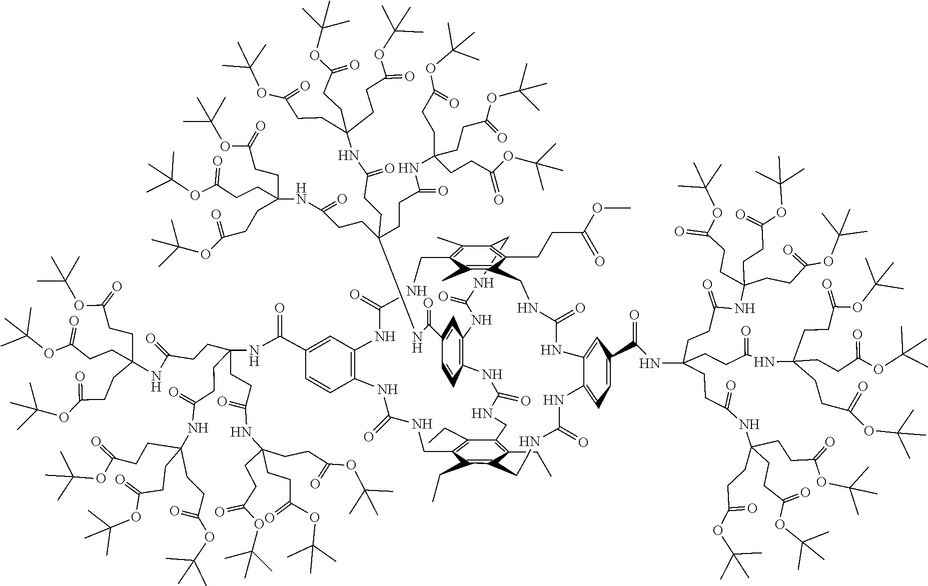

Prepared according to General Procedure F from “3-G2_TEB_HR” (synthesised according to compound 108 of WO 2018/167503, incorporated herein by reference) (430 mg, 0.085 mmol) and GI34 (49 mg, 0.136 mmol) in pyridine (32 mL), DMF (4 mL) and toluene (3 mL). Purified by RP MPLC (H₂O with increasing acetone) to give the product as a white solid (345 mg, 0.064 mmol, 75%).

¹H NMR (400 MHz, Methanol-d₄) δ 8.11-7.93 (m, 6H), 7.69-7.61 (m, 3H), 7.45 (s, 1H), 4.61-4.48 (m, 12H), 3.64 (s, 3H), 2.89 (s, 2H), 2.78-2.66 (m, 6H), 2.53 (s, 2H), 2.46 (s, 6H), 2.25-2.15 (m, 90H), 1.95 (dd, J=10.4, 5.8 Hz, 54H), 1.43 (s, 243H), 1.22 (q, J=7.2, 6.1 Hz, 9H).

### Synthesis of GI52

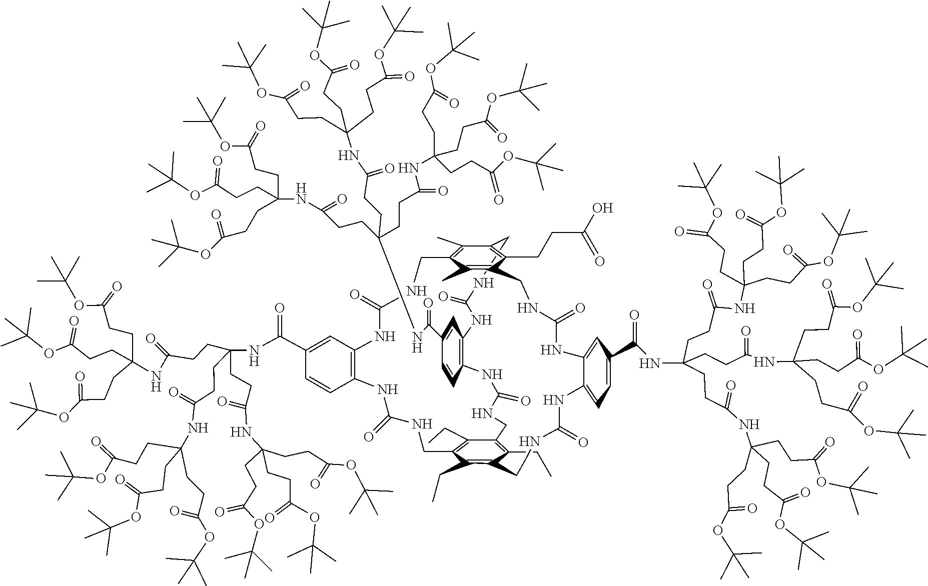

GI51 (100 mg, 18.5 μmol) dissolved in THF (2 mL) and sodium trimethylsilanolate (1 m in THF, 0.04 mL, 40.0 μmol) added. Reaction stirred for 16 h, then neutralised with 1 m aq. HCl. Solvents removed under reduced pressure and residue purified by RP MPLC (H₂O with increasing acetone) to give the product as a white solid (51 mg, 8.0 μmol, 44%).

¹H NMR (400 MHz, Methanol-d₄) δ 8.11-7.92 (m, 6H), 7.70-7.56 (m, 3H), 7.46 (d, J=5.9 Hz, 8H), 7.35 (s, 2H), 4.66-4.32 (m, 12H), 4.23-4.19 (m, 2H), 3.22 (s, 2H), 2.84-2.68 (m, 6H), 2.46 (s, 6H), 2.32-2.06 (m, 90H), 2.01-1.87 (m, 54H), 1.43 (s, 243H), 1.27-1.18 (m, 9H).

### Synthesis of GI53

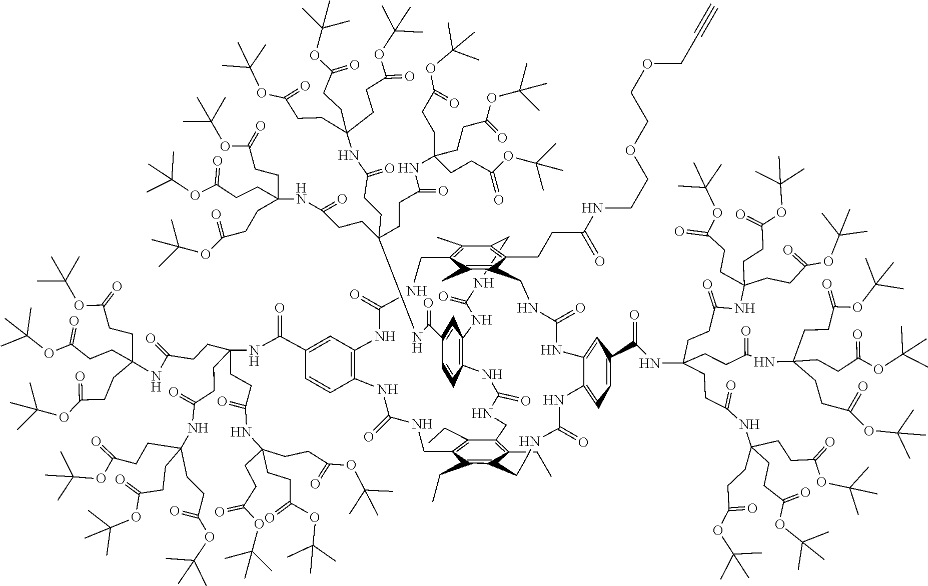

GI52 (51 mg, 9.5 μmol), EDC (2 mg, 12.0 μmol) and HOBt·H₂O (2 mg, 12.0 μmol) dissolved in DCM (0.6 mL). Et₃N (0.01 mL, 73.0 μmol) added and stirred for 30 minutes. 2-[2-(2-propynyloxy)ethoxy]ethylamine (8 mg, 48 μmol) added and reaction stirred for 16 hours. Solvents evaporated and the residue purified by RP MPLC (H₂O with increasing acetone) to give the product as a white solid (27 mg, 4.9 μmol, 52%).

¹H NMR (400 MHz, Methanol-d₄) δ 8.09-7.90 (m, 6H), 7.68 (d, J=8.5 Hz, 3H), 7.46 (s, 7H), 4.66-4.31 (m, 12H), 4.14 (d, J=2.4 Hz, 1H), 3.74-3.45 (m, J=32.1 Hz, 6H), 3.18 (s, 5H), 2.76 (s, 6H), 2.53-2.41 (m, 7H), 2.31-2.07 (m, 90H), 1.95 (t, J=8.0 Hz, 54H), 1.43 (s, 243H), 1.25-1.18 (m, 9H).

### Synthesis of GI54

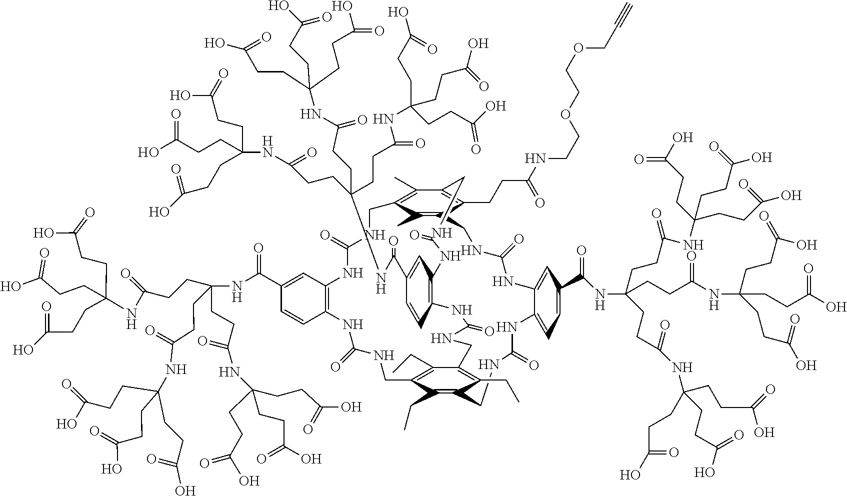

Prepared according to General Procedure D from GI53 (27 mg, 4.9 μmol) in TFA (0.3 mL) and DCM (0.6 mL). Product obtained as a white solid (19 mg, 4.7 μmol, 97%).

MS (electrospray, +ve), [M+3H]³⁺ calculated for C₁₃₃H₂₅₆N₂₅O₇₅ requires: 1334.9001, found: 1335.0.

### Synthesis of GI55

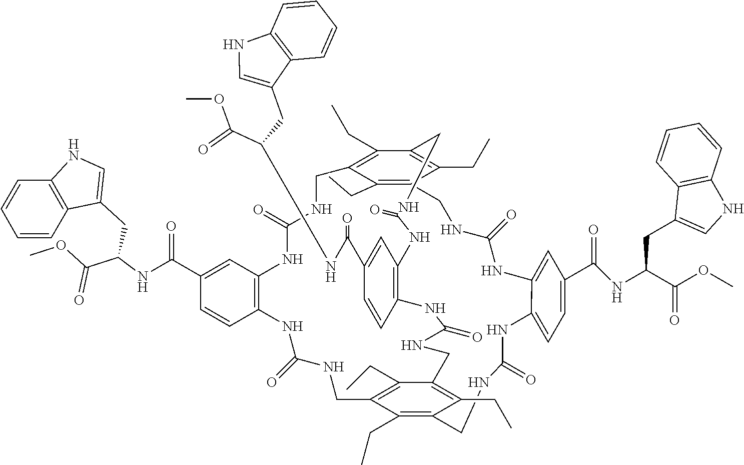 [SMILES: CCc1c2c(CC)c3c(CC)c1CNC(=O)Nc1ccc(C(=O)N[C@@H](Cc4c[nH]c5ccccc45)C(=O)OC)cc1NC(=O)NCc1c(CC)c(c(CC)c(c1CC)CNC(=O)Nc1cc(C(=O)N[C@@H](Cc4c[nH]c5ccccc45)C(=O)OC)ccc1NC(=O)NC3)CNC(=O)Nc1cc(C(=O)N[C@@H](Cc3c[nH]c4ccccc34)C(=O)OC)ccc1NC(=O)NC2]

Prepared according to General Procedure A from GBM16 (500 mg, 0.405 mmol), L-Trp-OMe (413 mg, 1.62 mmol), HBTU (538 mg, 1.42 mmol), HOBt·H₂O (248 mg, 1.62 mmol) and DIPEA (0.74 mL, 1.62 mmol) in THF (14 mL). Product purified by RP MPLC (water with increasing acetone) and isolated as a mixture of twice and thrice substituted products (417 mg).

### Synthesis of GI56

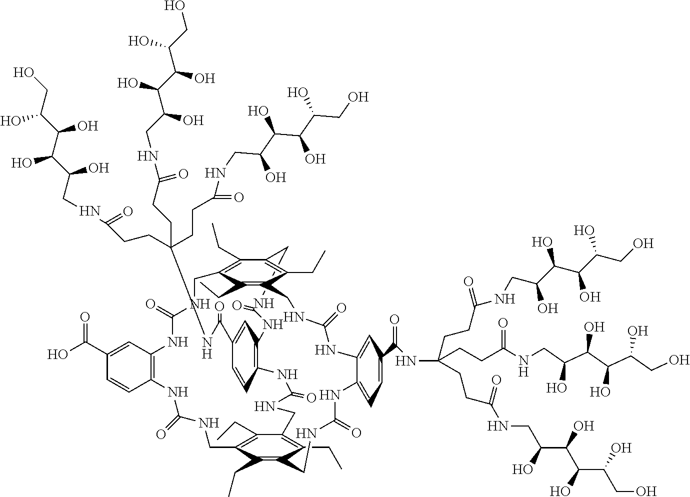

GI61 (106 mg, 0.041 mmol) dissolved in water (1 mL), NaOH (18 mg, 0.450 mmol) added, and reaction stirred at 40° C. for 16 hours. Neutralised with 1 M HCl and lyophilised to give the product (+NaCl) as a white solid.

MS (electrospray, +ve), [M+2H]²⁺ calculated for C₁₁₃H₁₇₆N₂₀O₄₆ requires: 1275.1035, found: 1275.2

### Synthesis of GI57

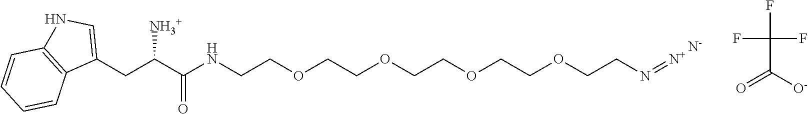

GI81 (347 mg, 0.632 mmol) dissolved in DCM (1.5 mL). TFA (0.5 mL) added and reaction stirred for 4 hours. DCM removed under a stream of N₂ and product precipitated with Et₂O. Supernatant decanted and residue dried under high vacuum to give the product as a gum (325 mg, 0.578 mmol, 91%).

¹H NMR (400 MHz, DMSO-d₆) δ 11.00 (d, J=2.4 Hz, 1H), 8.56 (t, J=5.4 Hz, 1H), 8.06 (s, 3H), 7.65 (d, J=7.9 Hz, 1H), 7.37 (d, J=8.0 Hz, 1H), 7.19 (d, J=2.4 Hz, 1H), 7.10 (t, J=7.6 Hz, 1H), 7.02 (t, J=7.4 Hz, 1H), 3.65-3.42 (m, 20H), 3.26-2.99 (m, 4H).

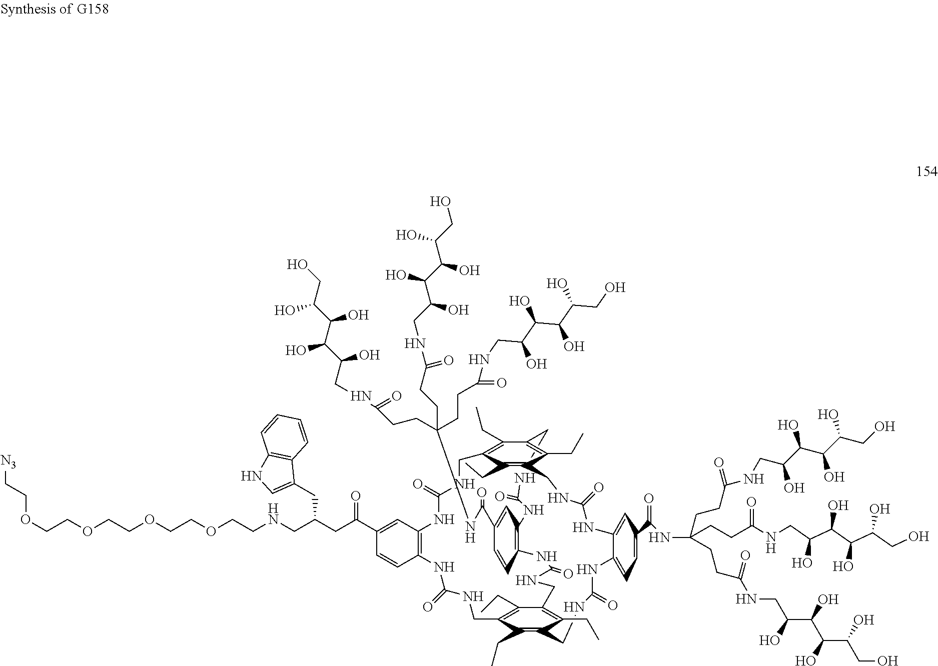

Prepared according to General Procedure A from GI56 (35 mg, 0.014 mmol), GI57 (16 mg, 0.027 mmol), HBTU (12 mg, 0.032 mmol), HOBt·H₂O (5 mg, 0.032 mmol) and DIPEA (0.01 mL, 0.055 mmol) in Pyr (1 mL). Product purified by RP MPLC (H₂O with increasing MeCN) to give the product as a white solid (19 mg, 6.0 μmol, 46%).

MS (electrospray, +ve), [M+2H]²⁺ calculated for C₁₃₄H₂₀₆N₂₆O₅₀ requires: 1490.2200, found: 1490.2.

### Synthesis of GI59

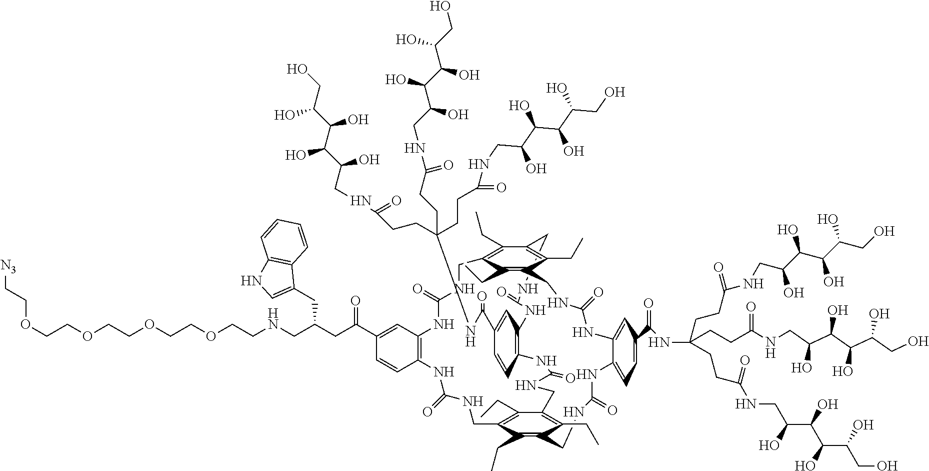

GI58 (18 mg, 6.0 μmol) dissolved in THF (2 mL) and water (5 mL), 1 M PMe₃ in THF (0.03 mL, 30 μmol) added and stirred for 17 hours. Solvents removed under a flow of N₂, redissolved in THF and evaporated to dryness again, to give the product as a white solid (18 mg, 6.0 μmol, 100%).

MS (electrospray, +ve), [M+2H]²⁺ calculated for C₁₃₄H₂₀₃N₂₄O₅₀ requires: 1477.2247, found: 1477.2

### Synthesis of GI60

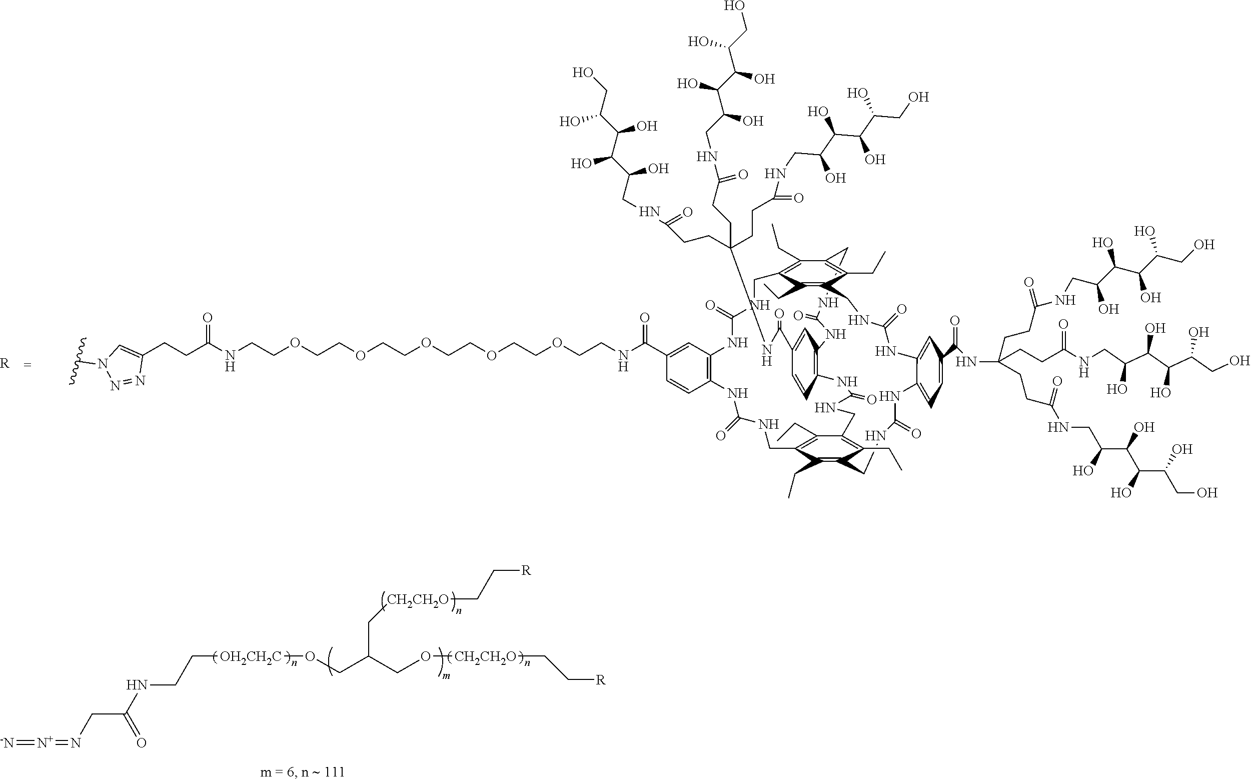

8-arm 40 kDa PEG-azide(7)/amime(1) (5 mg, 0.125 μmol) and sodium ascorbate (0.5 mg, 2.5 μmol) were dissolved in a solution of GBM1 (2.5 mg, 0.875 μmol) in water (0.6 mL). A solution of CuSO₄·5H₂O (0.2 mg, 0.625 μmol) in water (0.4 mL) was added and the reaction stirred at RT overnight. A solution of azidoacetic acid N-hydroxysuccinimidyl ester (0.3 mg, 1.5 μmol) in MeCN (0.3 mL) was added and stirred at RT for 2 days. The pH of the reaction was adjusted to pH 9 with saturated aqueous NaHCO₃ and purified by dialysis (10k MWCO) in pure water for 24 hours. The purified product solution was lyophilised to give the product as a yellow solid (7 mg, 0.10 μmol, 80%).

Absₘₐₓ=270 nm.

### Synthesis of GI61

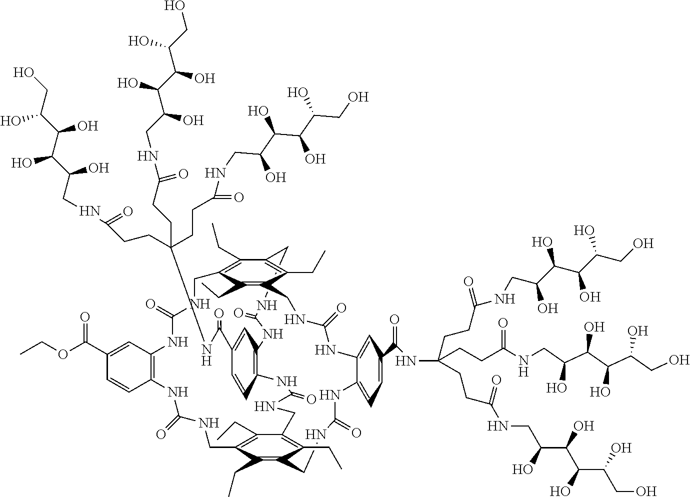

GI62 (365 mg, 0.171 mmol), HBTU (1.56 g, 4.11 mmol), HOBt·H₂O (629 mg, 4.11 mmol) and DIPEA (0.24 mL, 1.37 mmol) were dissolved in pyridine (5.5 mL). The reaction was stirred at RT for 15 minutes and D-glucamine (745 mg, 4.11 mmol) added, followed by a drop of water. The reaction was stirred at 45° C. for 2 hours and the solvent removed under vacuum. The crude solid was diluted with water (5 mL) and acetone (40 mL) was added to give a precipitate that was isolated by centrifuge. The crude solid was dissolved in water and purified by RP MPLC (H₂O with increasing MeCN (0.1% formic acid)) and the product fractions lyophilised to give the product (350 mg, 0.136 mmol, 79%) as a white solid.

¹H NMR (400 MHz, DMSO-d₆+1% D₂0) δ 8.15-8.07 (m, 3H), 7.95 (d, J=8.7 Hz, 1H), 7.91-7.76 (m, 7H), 7.69-7.53 (m, 3H), 7.42 (d, J=8.4 Hz, 1H), 6.55-6.28 (m, 6H), 4.41-4.09 (m, 14H), 3.61-3.47 (m, 18H), 3.44 (ddd, J=9.0, 5.8, 3.2 Hz, 6H), 3.34 (dt, J=10.4, 4.3 Hz, 12H), 3.27-3.15 (m, 6H), 3.04-2.93 (m, 6H), 2.83-2.51 (m, 12H), 2.06 (s, 12H), 1.92 (s, 12H), 1.24 (t, J=7.1 Hz, 3H), 1.07 (s, 18H).

Value of some integrals reduced due to deuterium-proton exchange from addition of D₂0.

MS (electrospray, +ve), [M+2H]²⁺ calculated for C₁₁₅H₁₃₀N₂₀O₄₆ requires: 1289.1192, found: 1289.1

### Synthesis of GI62

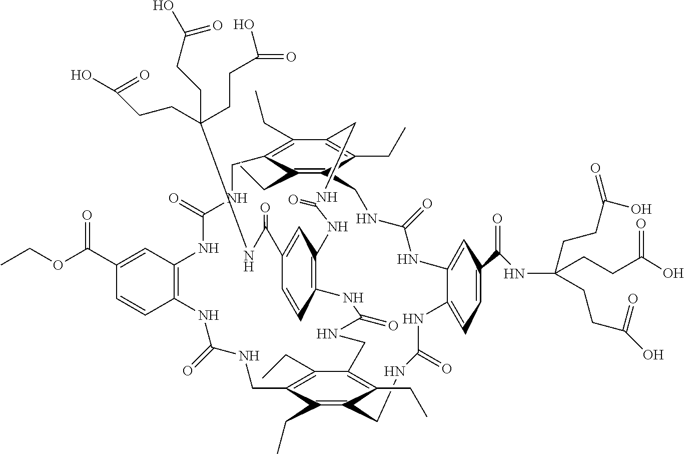

Prepared according to General Procedure D from GI63 (1.50 g, 0.775 mmol) in TFA (14 mL) and DCM (28 mL). Product obtained as a white solid after suspension in water and lyophilisation (1.21 g, 0.775 mmol, 98%).

¹H NMR (400 MHz, DMSO-d₆) δ 8.19 (d, J=2.0 Hz, 1H), 8.06 (d, J=8.7 Hz, 1H), 8.03 (d, J=1.9 Hz, 2H), 7.89 (d, J=8.6 Hz, 2H), 7.86 (s, 1H), 7.72 (s, 2H), 7.57 (dd, J=8.6, 2.1 Hz, 1H), 7.50 (s, 1H), 7.47-7.40 (m, 3H), 7.33 (s, 2H), 6.46 (s, 1H), 6.38 (s, 2H), 6.30 (s, 3H), 4.25 (dd, J=15.7, 7.1 Hz, 14H), 2.76 (s, 6H), 2.65 (s, 6H), 2.14 (t, J=8.0 Hz, 12H), 1.96 (d, J=9.0 Hz, 12H), 1.28 (t, J=7.1 Hz, 3H), 1.10 (d, J=7.6 Hz, 18H).

MS (electrospray, +ve), [M+H]⁺ calculated for C₇₉H₁₀₁N₁₄O₂₂ requires: 1597.7209, found: 1597.6

### Synthesis of GI63

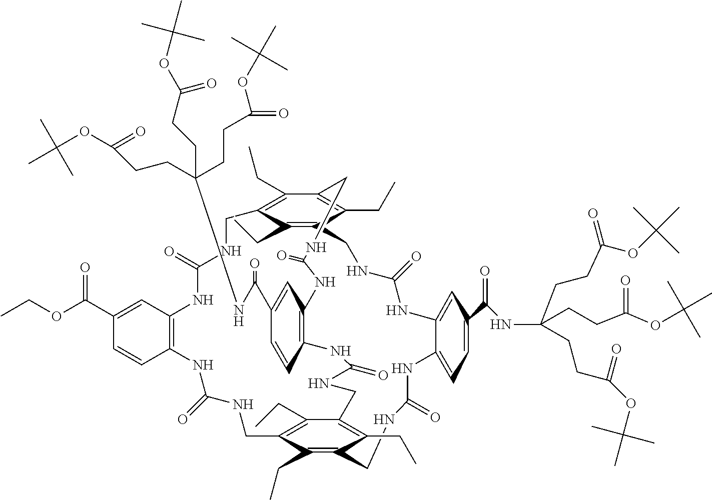

Prepared according to General Procedure A from GI64 (1.90 g, 1.668 mmol), di-tert-butyl 4-amino-4-[2-(tert-butoxycarbonyl)ethyl]heptanedioate (4.33 g, 8.33 mmol), HBTU (2.53 g, 6.671 mmol), HOBt·H₂O (1.02 g, 6.671 mmol) and DIPEA (1.8 mL, 10.0 mmol) in DMF (17 mL) and pyridine (17 mL) at 50° C. Purified by RP MPLC (H₂O with increasing acetone) to give the product as a white solid (1.6 g, 0.83 mmol, 50%).

¹H NMR (400 MHz, DMSO-d₆) δ 8.23 (t, J=2.0 Hz, 1H), 8.00 (d, J=8.6 Hz, 1H), 7.93 (d, J=8.5 Hz, 2H), 7.88 (d, J=2.1 Hz, 2H), 7.82-7.76 (m, 1H), 7.65 (s, 2H), 7.54 (dd, J=8.6, 2.2 Hz, 2H), 6.21 (q, J=19.0, 16.8 Hz, 6H), 4.48 (s, 6H), 4.43 (s, 6H), 4.35 (q, J=7.1 Hz, 2H), 2.97-2.68 (m, 12H), 2.32-2.23 (m, 12H), 2.12-2.07 (m, 12H), 1.45 (s, 54H), 1.39 (t, J=7.1 Hz, 3H), 1.27-1.13 (m, 18H).

MS (electrospray, +ve), [M+H]⁺ calculated for C₁₀₃H₁₄₈N₁₄O₂₂ requires: 1935.0999, found: 1935.0

### Synthesis of GI64

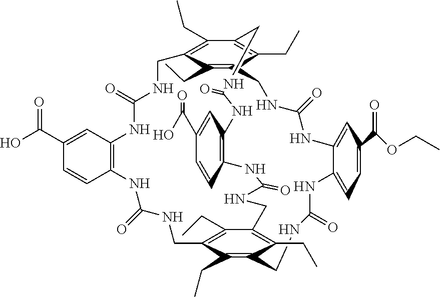

GI65 (6.00 g, 5.02 mmol) and sodium hydroxide (0.44 g, 11.0 mmol) were suspended inEtOH (220 mL) and water (60 mL) and stirred at 40° C. overnight. The organic solvent was removed under vacuum and 1 M HCl (300 mL) added to give a precipitate which was filtered, washed with water and air dried. The crude solid was adsorbed onto C18 silica gel and purified by RP MPLC (water with increasing MeCN (0.1% formic acid)). The solvent was removed under vacuum and dried under high vacuum to give the product (3.00 g, 2.57 mmol, 51%) as a white solid.

¹H NMR (400 MHz, DMSO-d₆) δ 8.18 (d, =2.1 Hz, 1H), 8.17 (d, J=2.1 Hz, 2H), 8.04 (d, J=8.7 Hz, 1H), 8.01 (d, J=8.6 Hz, 2H), 7.86 (s, 2H), 7.83 (s, 1H), 7.59-7.52 (m, 3H), 7.50 (s, 2H), 7.46 (s, 1H), 6.48-6.42 (m, 3H), 6.36-6.27 (m, 3H), 4.33-4.21 (m, 14H), 2.83-2.71 (m, 6H), 2.68-2.58 (m, 6H), 1.28 (t, J=7.1 Hz, 3H), 1.11 (td, J=7.3, 6.9, 2.4 Hz, 18H).

MS (electrospray, +ve), [M+H]⁺ calculated for C₅₉H₇₁N₁₂O₁₂ requires: 1139.5309, found: 1139.5

### Synthesis of GI65 (Triester macro)

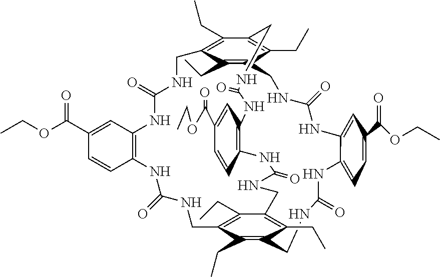

Synthesis described in Ziylo patent: WO2020058322A1 Glucose sensitive insulins and uses thereof

### Synthesis of GI66

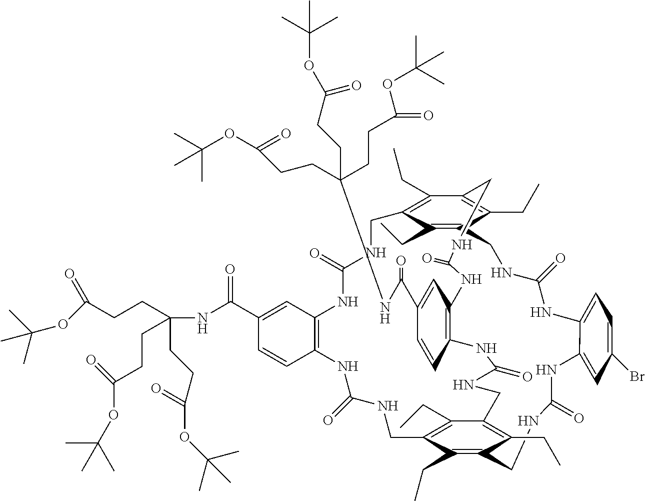

Prepared according to General Procedure F from GI67 (970 mg, 0.601 mmol) and “TEB NCO” (synthesised according to compound 103 of WO 2018/167503, incorporated herein by reference) (245 mg, 0.748 mmol) in pyridine (120 mL), DMF (35 mL) and DCM (10 mL). Purified by RP MPLC (H₂O with increasing acetone and H₂O with increasing MeOH) to give the product as a white solid (701 mg, 0.361 mmol, 60%).

¹H NMR (400 MHz, Methanol-d₄) δ 8.00-7.91 (m, 3H), 7.87 (d, J=2.1 Hz, 2H), 7.66 (s, 2H), 7.61-7.51 (m, 3H), 7.22 (dd, J=8.7, 2.3 Hz, 1H), 4.45 (d, J=21.8 Hz, 13H), 2.98-2.69 (m, 13H), 2.28 (dd, J=9.7, 6.2 Hz, 13H), 2.10 (dd, J=9.5, 6.4 Hz, 13H), 1.45 (s, 59H), 1.28-1.13 (m, 19H).

### Synthesis of GI67

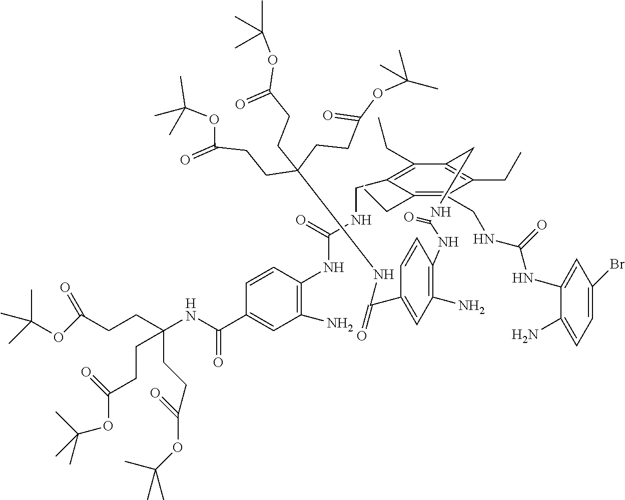 [SMILES: CCc1c(CNC(=O)Nc2ccc(C(=O)NC(CCC(=O)OC(C)(C)C)(CCC(=O)OC(C)(C)C)CCC(=O)OC(C)(C)C)cc2N)c(CC)c(CNC(=O)Nc2cc(Br)ccc2N)c(CC)c1CNC(=O)Nc1ccc(C(=O)NC(CCC(=O)OC(C)(C)C)(CCC(=O)OC(C)(C)C)CCC(=O)OC(C)(C)C)cc1N]

Dissolved GI68 (1.890 g, 0.829 mmol) in DCM (40.0 mL) and added DBU (0.743 mL, 4.972 mmol, 6.000 eq) at RT. After 30 minutes the solvent was removed and the resulting oil was dissolved in MeCN for RP MPLC (H₂O with increasing acetone) to give the product as a white solid (0.97 g, 0.601 mmol, 73%).

¹H NMR (400 MHz, DMSO-d₆) δ 7.73 (d, J=2.3 Hz, 1H), 7.67 (s, 2H), 7.61-7.56 (m, 3H), 7.18 (s, 2H), 7.13 (d, J=2.1 Hz, 2H), 7.04 (dd, J=8.4, 2.0 Hz, 2H), 6.89 (dd, J=8.5, 2.4 Hz, 1H), 6.64 (d, J=8.5 Hz, 1H), 6.34 (s, 3H), 4.73 (d, J=10.1 Hz, 6H), 4.37 (s, 6H), 2.81 (s, 6H), 2.11 (q, J=7.7 Hz, 13H), 1.91 (t, J=8.1 Hz, 12H), 1.37 (s, 58H), 1.19 (t, J=7.1 Hz, 1 OH).

### Synthesis of GI68

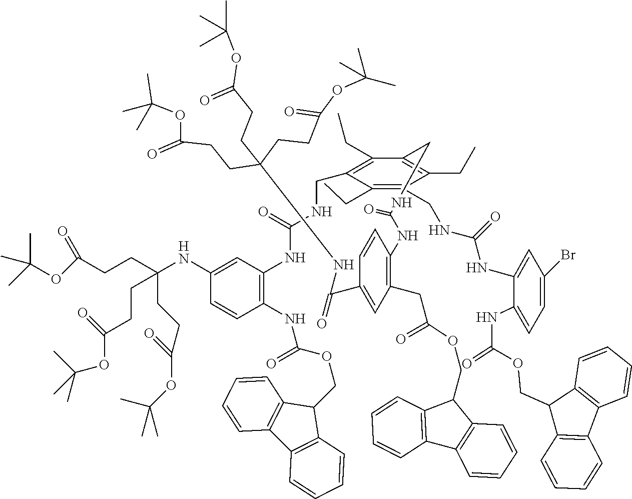 [SMILES: CCc1c(CNC(=O)Nc2ccc(C(=O)NC(CCC(=O)OC(C)(C)C)(CCC(=O)OC(C)(C)C)CCC(=O)OC(C)(C)C)cc2CC(=O)OCC2c3ccccc3-c3ccccc32)c(CC)c(CNC(=O)Nc2cc(NC(CCC(=O)OC(C)(C)C)(CCC(=O)OC(C)(C)C)CCC(=O)OC(C)(C)C)ccc2NC(=O)OCC2c3ccccc3-c3ccccc32)c(CC)c1CNC(=O)Nc1cc(Br)ccc1NC(=O)OCC1c2ccccc2-c2ccccc21]

Dissolved “G1M linker” (synthesised according to compound 2 of WO 2018/167503, incorporated herein by reference) (3.207 g, 4.154 mmol, 2.000 eq) and “TEB NCO” (synthesised according to compound 103 of WO 2018/167503, incorporated herein by reference) (0.680 g, 2.077 mmol) in anhydrous DMF (40.0 mL) under nitrogen at RT.

After 8 hours added a solution of GI69 in dry DMF (8 mL). After 3 days the reaction mixture was evaporated to give8.3 g of a thick dark brown oil. Crude adsorbed onto C18 and partitioned in three to be purified by RP MPLC (H₂O with increasing acetone) to give the desired product as a white solid (1.99 g, 0.873 mmol, 42%).

¹H NMR (400 MHz, DMSO-d₆) δ 8.22 (s, 2H), 8.04 (s, 2H), 7.71 (d, J=7.2 Hz, 6H), 7.61 (s, 8H), 7.36-7.24 (m, 8H), 7.17 (s, 7H), 7.01 (s, 1H), 4.31 (s, 10H), 2.71 (s, 6H), 2.15-2.04 (m, 11H), 1.92 (t, J=8.0 Hz, 11H), 1.30 (s, 51H), 1.11 (d, J=26.0 Hz, 9H).

### Synthesis of GI69

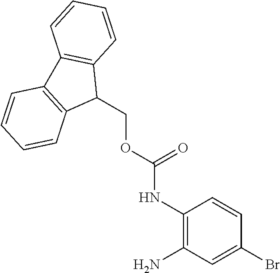 [SMILES: Nc1cc(Br)ccc1NC(=O)OCC1c2ccccc2-c2ccccc21]

Dissolved diaminobromobenzene (5.000 g, 26.732 mmol) in MeCN (150.0 mL) and then added the Fmoc-OSu (9.919 g, 29.405 mmol, 1.100 eq). The solution was stirred at RT overnight. The next day, the reaction mixture was filtered with a sintered funnel and washed with MeCN. This gave a fine tan coloured powder which can be further purified by recrystallization in an EtOH/DCM mixture (4.57 g, 11.16 mmol, 42%).

¹H NMR (400 MHz, Deuterium Oxide) δ 8.77 (s, 1H), 7.90 (d, J=7.5 Hz, 2H), 7.73 (s, 2H), 7.42 (t, J=7.4 Hz, 2H), 7.34 (t, J=7.5 Hz, 2H), 7.10 (s, 1H), 6.89 (d, J=2.3 Hz, 1H), 6.65 (d, J=7.8 Hz, 1H), 4.41 (d, J=6.9 Hz, 2H), 4.29 (d, J=7.3 Hz, 1H), 2.50 (p, J=1.8 Hz, 1H).

### Synthesis of GI70

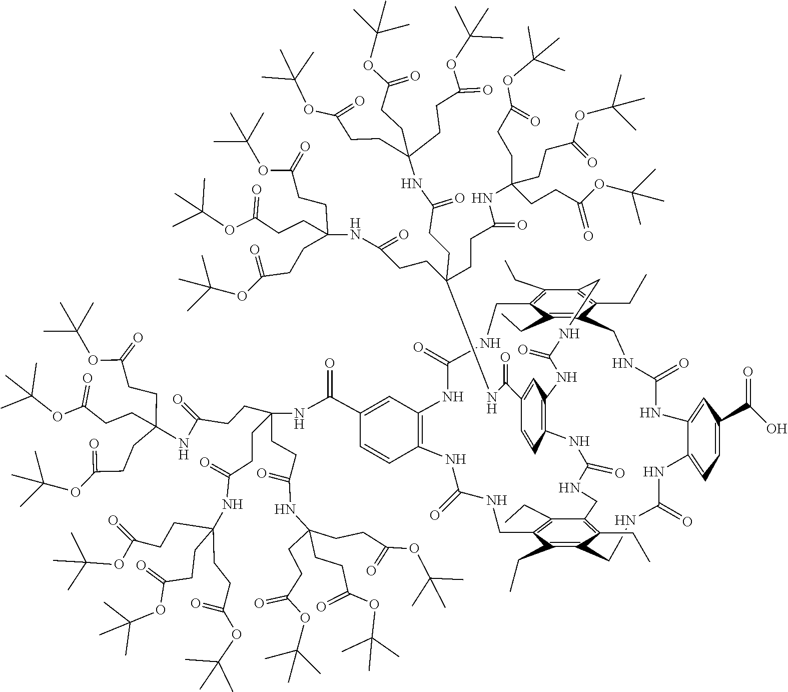

To a solution of GI71 (500 mg, 0.124 mmol) in EtOH (5 mL) and THF (5 mL) was added Pd/C (50 mg, 10% w/w). Reaction stirred overnight under an atmosphere of hydrogen at RT. Filtered through celite with THF/H₂O and concentrated. Purified by RP MPLC (H₂O with increasing acetone). Product obtained as a white solid (254 mg, 0.064 mmol, 52%).

¹H NMR (400 MHz, Methanol-d₄) δ 8.30-7.77 (m, 6H), 7.66 (d, J=48.0 Hz, 3H), 4.73-4.05 (m, 12H), 3.02-2.40 (m, 12H), 2.34-2.01 (m, 60H), 1.92 (t, J=8.1 Hz, 36H), 1.47-1.34 (m, 162H), 1.34-1.09 (m, 18H).

### Synthesis of GI71

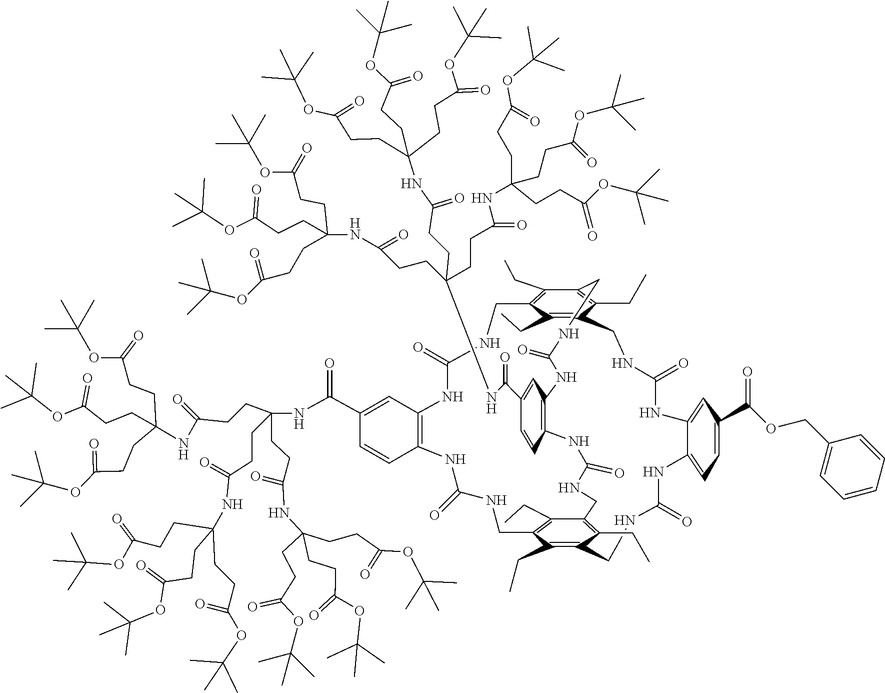

GI72 (569 mg, 0.355 mmol), HOBt·H₂O (217 mg, 1.420 mmol), HBTU (539 mg, 1.420 mmol) dissolved in THF (10 mL) with DIPEA (0.5 mL, 2.840 mmol). After stirring for 15 minutes G2MM amine (1.07 g, 0.746 mmol) added. The reaction was left to stir for 66 hours. Reaction mixture was concentrated under vacuum and then purified using RP MPLC (H₂O with increasing acetone). Product obtained as a white solid (510 mg, 0.126 mmol, 36%).

¹H NMR (400 MHz, DMSO-d₆) δ 8.02 (s, 3H), 7.91 (d, J=8.2 Hz, 3H), 7.73 (s, 4H), 7.47 (d, J=8.7 Hz, 2H), 7.44-7.29 (m, 8H), 7.24 (s, 6H), 6.52-6.21 (m, 6H), 5.29 (s, 2H), 4.26 (s, 12H), 2.80-2.57 (m, 12H), 2.09-1.99 (m, 42H), 1.89 (s, 18H), 1.75 (s, 36H), 1.33 (s, 162H), 1.10 (d, J=8.4 Hz, 18H).

### Synthesis of GI72

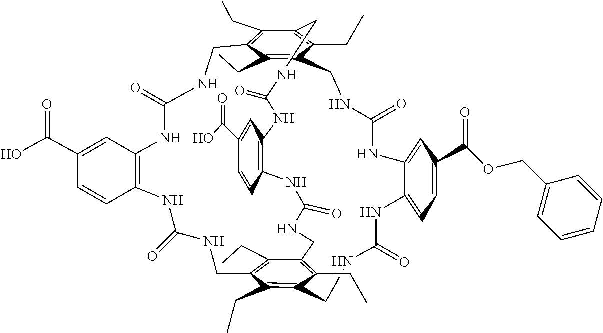

GBM16 (100 mg, 0.090 mmol) and CsCO₃ (59 mg, 0.180 mmol) stirred in DMF (1.8 mL) for 10 minutes. BnBr (21 μL, 0.180 mmol) added and the reaction stirred for hours. Reaction mixture was concentrated under vacuum and then purified using RP MPLC (H₂O with increasing acetone). Mixture of macrocycles produced, product of interest obtained as a white solid (41 mg, 0.034 mmol, 38%).

1H NMR (400 MHz, DMSO-d₆) δ 8.46-8.22 (m, 6H), 8.16-7.94 (m, 6H), 7.63-7.46 (m, 5H), 7.38 (ddd, J=23.2, 15.2, 6.9 Hz, 5H), 6.48 (s, 3H), 6.25 (s, 3H), 5.28 (s, 2H), 4.28 (s, 12H), 2.63 (d, J=32.2 Hz, 12H), 1.11 (q, J=5.4, 3.7 Hz, 18H).

### Synthesis of GI73

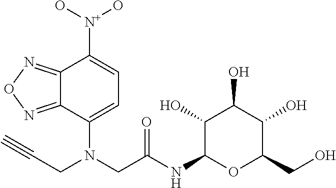 [SMILES: C#CCN(CC(=O)N[C@@H]1O[C@H](CO)[C@@H](O)[C@H](O)[C@H]1O)c1ccc([NH+](O)O)c2nonc12]

GI74 (35 mg, 0.128 mmol) and NBD-chloride (26 mg, 0.128 mmol) dissolved in DMSO (1 mL) and DIPEA (22 μL, 0.128 mmol) added. Stirred for 2 hours then solvents concentrated under a stream of N₂. Residue purified by RP MPLC (H₂O with increasing MeCN (0.1% formic acid)) to give the product as an orange solid (22 mg, 0.050 mmol, 39%).

1H NMR (400 MHz, DMSO-d₆) δ 8.80 (d, J=9.1 Hz, 1H), 8.60 (d, J=8.9 Hz, 1H), 6.57 (d, J=9.1 Hz, 1H), 5.02 (d, J=4.7 Hz, 1H), 4.93 (dd, J=15.4, 4.9 Hz, 2H), 4.84-4.62 (m, 5H), 4.49 (t, J=5.6 Hz, 1H), 3.63 (dd, J=11.1, 5.7 Hz, 1H), 3.50 (t, J=2.3 Hz, 1H), 3.42 (s, 1H), 3.22-3.14 (m, 1H), 3.14-3.02 (m, 3H).

MS (electrospray, +ve) [M+Na]⁺ calculated for C₁₇H₁₉N₅NaO₉ requires: 460.1075, found: 460.1.

### Synthesis of GI74

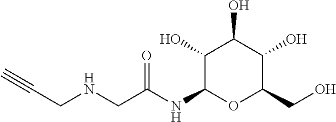 [SMILES: C#CCNCC(=O)N[C@@H]1O[C@H](CO)[C@@H](O)[C@H](O)[C@H]1O]

GI75 (100 mg, 0.267 mmol) dissolved in 4 M HCl, after 10 minutes the solution was basified with Na₂CO₃. Solution concentrated to give the product mixed with NaCl.

1H NMR (400 MHz, Deuterium Oxide) δ 5.00 (dd, J=9.1, 3.0 Hz, 1H), 4.08 (s, 2H), 3.88 (dt, J=12.5, 2.3 Hz, 1H), 3.72 (ddd, J=12.5, 5.4, 1.3 Hz, 1H), 3.55 (dd, J=3.8, 2.3 Hz, 1H), 3.53 (s, 1H), 3.53-3.51 (m, 1H), 3.50-3.47 (m, 1H), 3.46-3.42 (m, 3H).

### Synthesis of GI75

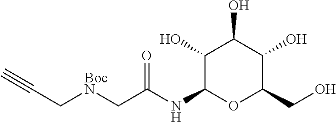 [SMILES: C#CCN(CC(=O)N[C@@H]1O[C@H](CO)[C@@H](O)[C@H](O)[C@H]1O)C(=O)OC(C)(C)C]

Prepared according to General Procedure A from GI76 (520 mg, 2.439 mmol), 1-aminoglucose (843 mg, 3.999 mmol), HBTU (1.11 g, 2.926 mmol), HOBt·H₂O (374 mg, 2.439 mmol) in DMF (5 mL) and Pyridine (5 mL). Product purified by RP MPLC (H₂O with increasing acetone) to give the product as a white solid (617 mg, 1.648 mmol, 68%).

1H NMR (400 MHz, Methanol-d₄) δ 4.90 (d, J=9.1 Hz, 1H), 4.11 (d, J=11.4 Hz, 2H), 4.06-3.94 (m, 2H), 3.80 (dd, J=11.9, 2.1 Hz, 1H), 3.68-3.58 (m, 1H), 3.38 (t, J=8.7 Hz, 1H), 3.24 (t, J=9.0 Hz, 1H), 2.66 (t, J=2.5 Hz, 1H), 1.45 (d, J=14.6 Hz, 9H).

### Synthesis of GI76

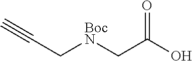

Boc-Gly-OH (5.000 g, 28.541 mmol) stirred in THF (100 mL) at 0° C. NaH (2.765 g, 69.143 mmol) added in three portions. Propargyl bromide (3.4 mL, 31.396 mmol) added to the resultant gel. Further THF (40 mL) and DMF (20 mL) and the reaction heated at 50° C. for 3 d. Reaction mixture diluted with EtOAc (200 mL) and washed with 1 M HCl (200 mL), water (3×200 mL) and brine (100 mL). Organics dried (Na₂SO₄) and concentrated to an oil. Product purified by MPLC (DCM with increasing EtOAc) to give the product as a brown solid (2.680 g, 12.568 mmol, 44%).

1H NMR (400 MHz, Chloroform-d) δ 8.91 (s, 1H), 4.24-4.08 (m, 4H), 2.27 (t, J=2.5 Hz, 1H), 1.48 (s, 4H), 1.44 (s, 5H).

### Synthesis of GI77

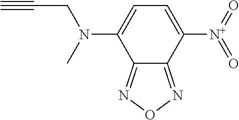

N-methylprop-2-yn-1-amine (14 mg, 0.203 mmol) and NBD-chloride (40 mg, 0.203 mmol) dissolved in DMSO (1 mL) and DIPEA (35 μL, 0.203 mmol) added. Stirred for 2 hours then further N-methylprop-2-yn-1-amine (7 mg, 0.101 mmol) added and stirred for 18 hours. Solvents concentrated under a stream of N₂. Residue purified by RP MPLC (H₂O with increasing MeCN (0.1% formic acid)) to give the product as an orange solid (22 mg, 0.050 mmol, 39%).

1H NMR (400 MHz, Methanol-d₄) δ 7.75 (d, J=8.8 Hz, 1H), 5.49 (s, 1H), 4.12 (s, 2H), 3.81 (s, 1H), 2.35 (s, 3H).

MS (electrospray, +ve) [2M+H]⁺ calculated for C₂₀H₁₇N₈O₆ requires: 465.1266, found: 465.1.

### Synthesis of GI78

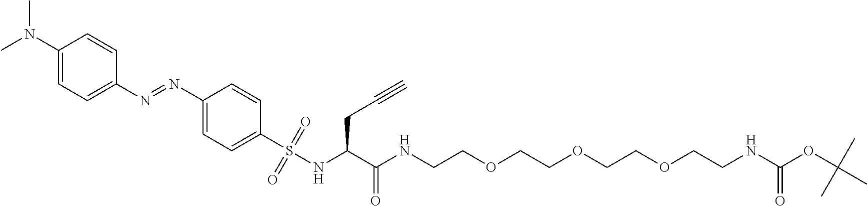 [SMILES: C#CC[C@H](NS(=O)(=O)c1ccc(/N=N/c2ccc(N(C)C)cc2)cc1)C(=O)NCCOCCOCCOCCNC(=O)OC(C)(C)C]

GI79 (9 mg, 0.023 mmol) and dabsyl chloride (11 mg, 0.035 mmol) dissolved in MeCN (1 mL) and DIPEA (8 μL, 0.046 mmol) added. Stirred for 16 hours then further dabsyl chloride (30 mg, 0.093 mmol) and DMAP (3 mg, 0.025 mmol) added and stirred for 2 d. Reaction then heated to 50° C. for 2 hours. Solvents removed under reduced pressure. Residue purified by RP MPLC (H₂O with increasing MeCN (0.1% formic acid)) to give the product (contaminated with dabsyl-OH) as an orange solid (14 mg).

1H NMR (400 MHz, Methanol-d₄) δ 8.10-8.07 (m, 4H), 7.02-6.91 (m, 4H), 3.97 (t, J=6.9 Hz, 1H), 3.65-3.46 (m, 10H), 3.20 (d, J=5.7 Hz, 4H), 3.18 (t, J=5.5 Hz, 2H), 3.13 (s, 6H), 2.52 (dt, J=6.9, 2.4 Hz, 2H), 2.31 (t, J=2.6 Hz, 1H), 1.42 (s, 9H).

MS (electrospray, +ve) [M+Na]⁺ calculated for C₃₂H₄₆N₆NaO₃S requires: 697.2991, found: 697.3.

### Synthesis of GI79

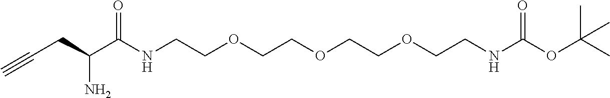 [SMILES: C#CC[C@H](N)C(=O)NCCOCCOCCOCCNC(=O)OC(C)(C)C]

To a suspension of GI80 (50 mg, 0.082 mmol) in DCM (2.5 mL) was added DBU (37 μL, 0.246 mmol). Reaction stirred for 2 hours at RT before solvents were removed under vacuum. Purified by RP MPLC (H₂O with increasing MeCN (0.1% formic acid)). Product obtained as a clear oil (9 mg, 0.023 mmol, 28%).

1 H NMR (400 MHz, Chloroform-d) δ 8.20 (d, J=75.3 Hz, 3H), 3.99 (s, 1H), 3.66-3.34 (m, 16H), 2.79 (s, 2H), 2.22 (s, 1H), 1.42 (s, 9H).

### Synthesis of GI80

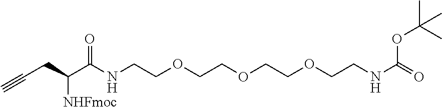

Fmoc-L-propargylglycine (150 mg, 0.447 mmol), EDC (129 mg, 0.671 mmol) and HOBt·H₂O (103 mg, 0.671 mmol) dissolved in DCM (26 mL) with DIPEA (0.12 mL, 0.685 mmol). After 30 min, N-Boc-1,11-diamino-3,6,9-trioxaundecane (196 mg, 0.671 mmol) added and the reaction stirred for 18 hours. Solvents removed under reduced pressure and the residue redissolved in EtOAc (100 mL) and washed with 5% aq. KHSO₄ (100 mL), sat. aq. NaHCO₃ (100 mL) and brine (100 mL) then dried (Na₂SO₄). Organic solvents removed in vacuo and the crude product purified by MPLC (H₂O with increasing MeCN (0.1% formic acid)) to give the product as a clear oil (125 mg, 0.205 mmol, 46%).

1H NMR (400 MHz, Chloroform-d) δ 7.76 (d, J=7.5 Hz, 2H), 7.60 (d, J=7.5 Hz, 2H), 7.40 (t, J=7.4 Hz, 2H), 7.36-7.30 (m, 2H), 6.80 (s, 1H), 5.84 (s, 1H), 5.12 (s, 1H), 4.45 (dd, J=10.5, 7.1 Hz, 1H), 4.38 (t, J=9.0 Hz, 2H), 4.23 (t, J=7.0 Hz, 1H), 3.60 (d, J=8.4 Hz, 10H), 3.51 (q, J=5.4 Hz, 4H), 3.28 (t, J=5.2 Hz, 2H), 2.77 (t, J=10.2 Hz, 1H), 2.71-2.53 (m, 1H), 2.16 (d, J=5.3 Hz, 1H), 1.43 (s, 9H).

MS (electrospray, +ve) [M+Na]⁺ calculated for C₃₃H₄₃N₃NaO₃ requires: 632.2943, found: 632.3.

### Synthesis of GI81

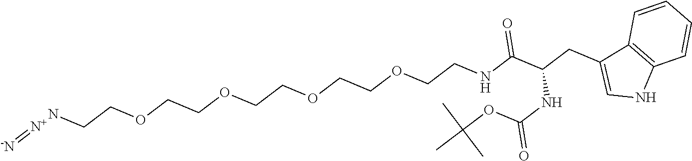

Nₐ-Boc-L-tryptophan (250 mg, 0.822 mmol) and HOBt·H₂O (188 mg, 1.23 mmol) were dissolved in DCM (48 mL). DIPEA (0.22 mL, 1.23 mmol) and EDC·HCl (236 mg, 1.23 mmol) were added and the reaction stirred at RT for 30 minutes. O-(2-aminoethyl)-O′-(2-azidoethyl)triethylene glycol (323 mg, 1.23 mmol) was added and the reaction stirred at RT for 1 hour. The reaction mixture was washed with NaHCO₃, brine, the organic phase dried (Na₂SO₄) and the solvent removed under vacuum. The crude solid was then purified by RP MPLC (H₂O with increasing MeCN (0.1% formic acid)). The solvent was then removed under vacuum to give the product (347 mg, 0.633 mmol, 77%) as a white solid.

1H NMR (400 MHz, Chloroform-d) δ 8.91 (s, 1H), 7.70 (d, J=7.9 Hz, 1H), 7.38 (d, J=8.0 Hz, 1H), 7.21-7.13 (m, 1H), 7.14-7.07 (m, 2H), 5.96 (s, 1H), 5.42 (s, 1H), 4.39 (s, 1H), 3.82-3.43 (m, 13H), 3.43-3.20 (m, 7H), 3.09 (d, J=11.4 Hz, 2H), 1.45 (s, 9H).

MS (electrospray, +ve) [M+Na]⁺ calculated for C₂₆H₄₀N₆O₇ requires: 571.2851, found: 571.2.

### Synthesis of GI82

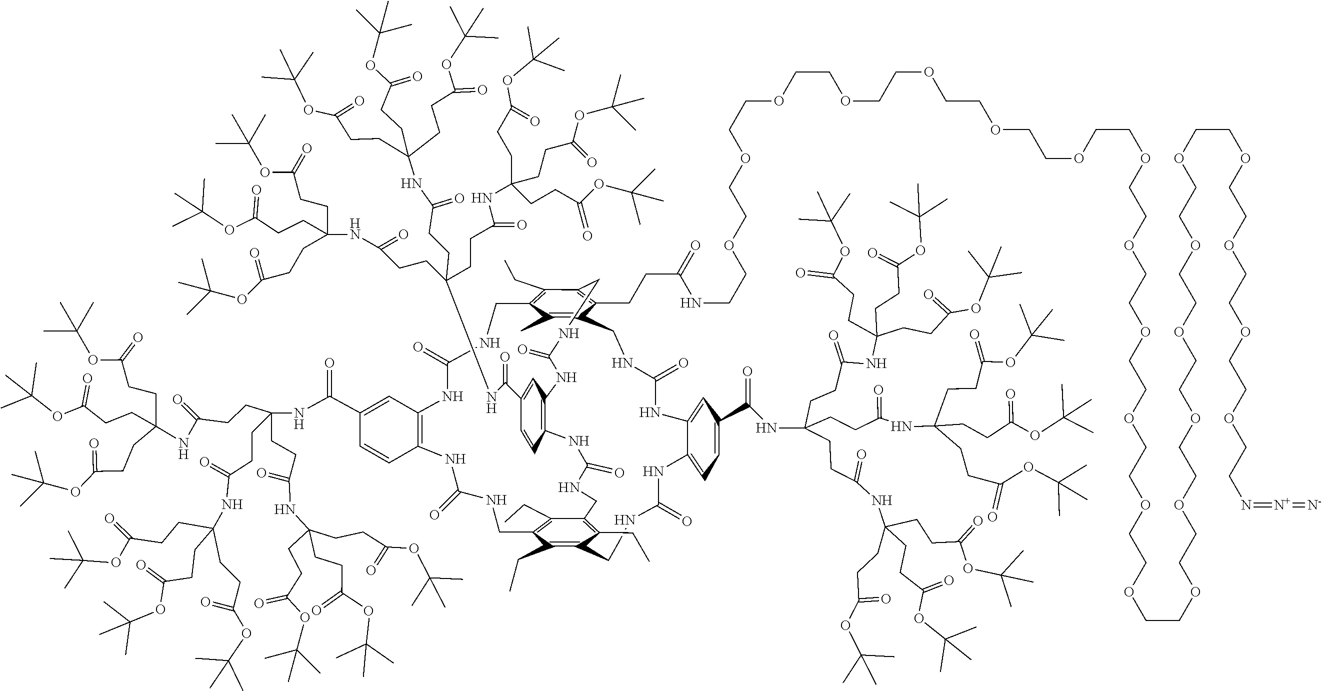

GI52 (40 mg, 7.4 μmol), EDC (1.8 mg, 9.4 μmol) and HOBt·H₂ (1.4 mg, 9.4 μmol) dissolved in DCM (0.5 mL). EtN (0.01 mL, 73.0 μmol) added and stirred for 30 min. azido-PEG₂₃-amine (18 mg, 15 μmol) added and reaction stirred for 16 hours. Solvents evaporated, and the residue purified by RP MPLC (H₂O with increasing acetone) to give the product as a white solid (28 mg, 4.3 μmol, 58%).

1H NMR (400 MHz, Methanol-d₄) δ 8.04 (s, 6H), 7.64 (d, J=8.8 Hz, 3H), 7.45 (s, 1H), 4.48 (d, J=50.9 Hz, 12H), 3.72-3.46 (m, 96H), 3.37 (d, J=4.7 Hz, 2H), 3.16-3.07 (m, 2H), 2.74 (s, 6H), 2.50-2.40 (m, 6H), 2.28-2.10 (m, 90H), 1.95 (t, J=7.9 Hz, 54H), 1.43 (d, J=2.0 Hz, 243H), 1.28-1.13 (m, 9H).

(Of sample globally hydrolysed with TFA) MS (electrospray, +ve), [M+3H]³⁺ calculated for C₂₂₄H₃₄₁N₂₃O₉₆ requires: 1653.7571, found: 1653.8.

### Synthesis of GI83

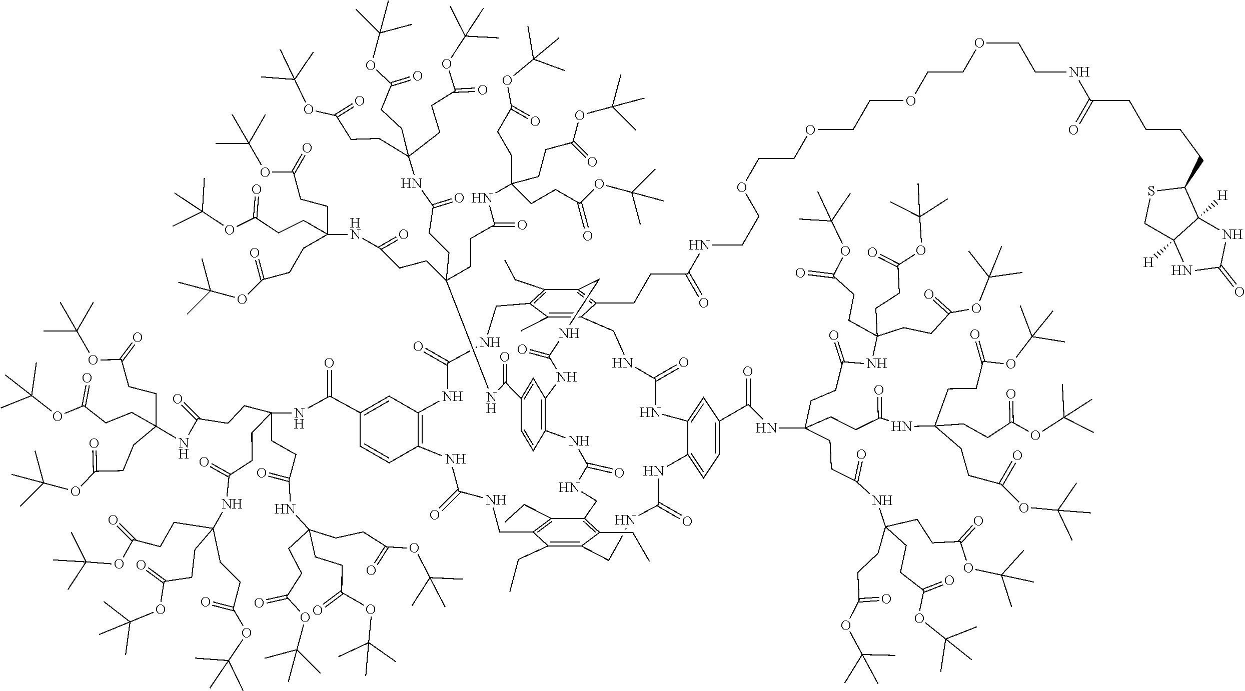

Prepared according to General Procedure C from GI84 (8 mg, 1.42 μmol), (+)-Biotin-N-hydroxysuccinimide ester (2 mg, 5.86 μmol), EtaN (0.05 mL, 0.36 mmol) in THF (1.5 mL). Solvents removed under a stream of N₂ and the product used without further purification.

### Synthesis of GI84

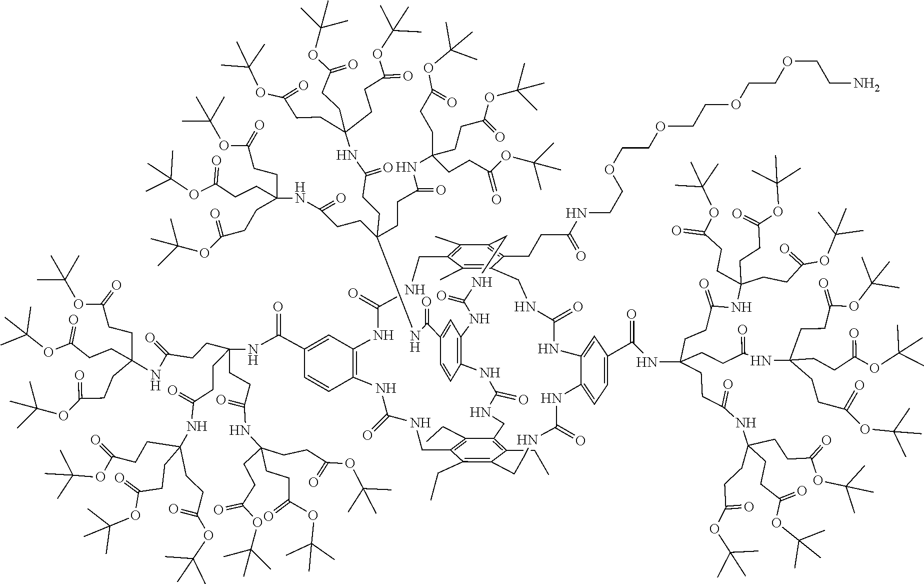

GI85 (8 mg, 1.42 μmol) dissolved in THF (1 mL) and water (0.3 mL), PMe₃ (1 M solution in THF, 0.01 mL, 10 μmol) added and stirred for 17 hours. Solvents removed under a flow of N₂, redissolved in THF and evaporated to dryness again, to give the product as a white solid (8 mg, 1.42 μmol, 100%).

(Of sample globally hydrolysed with TFA) MS (electrospray, +ve), [M+3H]³⁺ calculated for C₁₅₆H₂₈₇N₂₈O₇₇ requires: 1366.2609, found: 1366.0.

### Synthesis of GI85

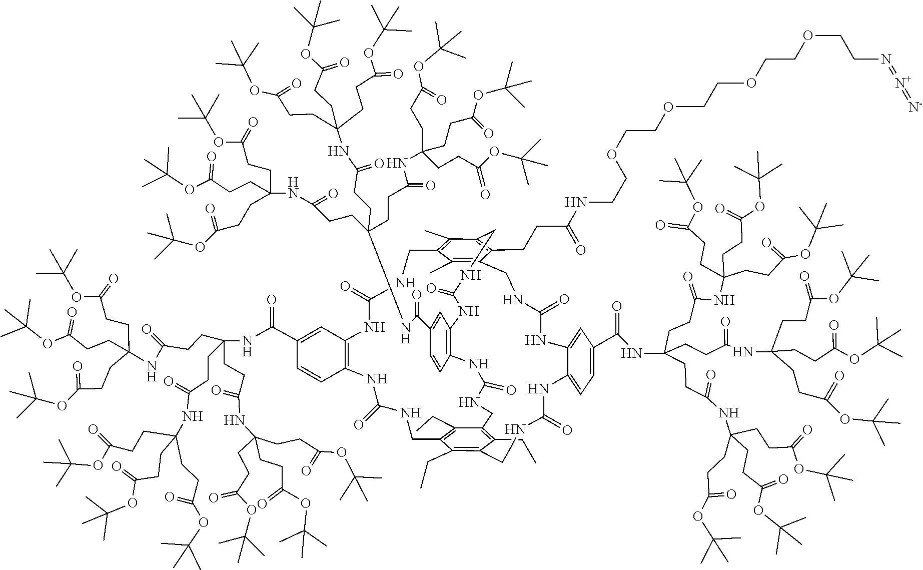

GI52 (18 mg, 3.34 μmol), EDC (1 mg, 5.22 μmol) and HOBt·H₂O (0.6 mg, 3.92 μmol) dissolved in DCM (1 mL). Et₃N (0.10 mL, 715 μmol) added and stirred for 30 min. azido-PEG₄-amine (5 mg, 19.1 μmol) added and reaction stirred for 48 hours. Solvents evaporated, and the residue purified by RP MPLC (H₂O with increasing acetone) to give the product as a white solid (8 mg, 1.42 μmol, 43%).

¹H NMR (400 MHz, Methanol-d₄) δ 8.21-7.93 (m, 9H), 7.65 (d, J=8.6 Hz, 3H), 7.50-7.41 (m, 9H), 4.66-4.28 (m, 12H), 3.76-3.34 (m, 12H), 3.27-3.07 (m, 10H), 2.80-2.56 (m, 8H), 2.54-2.41 (m, 6H), 2.33-2.05 (m, 90H), 1.95 (t, J=8.1 Hz, 54H), 1.43 (d, J=1.2 Hz, 243H), 1.21 (t, J=7.0 Hz, 9H).

(Of sample globally hydrolysed with TFA) MS (electrospray, +ve), [M+3H]³⁺ calculated for C₁₈₆H₂₆₅N₂₈O₇₇ requires: 1374.9244, found: 1374.9.

### Synthesis of GI86

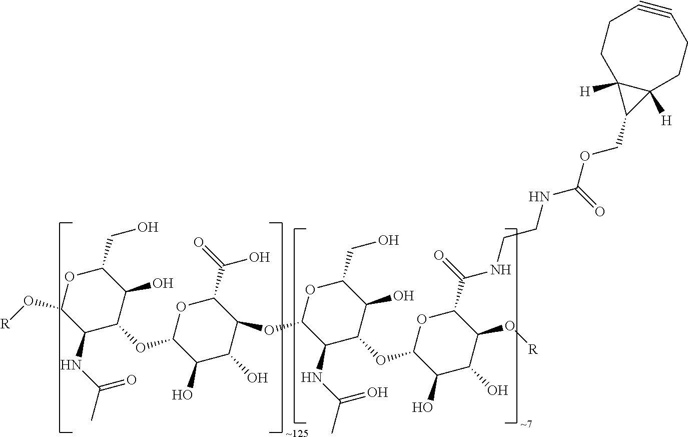

Hyaluronate amine (5%), MW 50k (21 mg, 0.42 μmol) and NaHCO₃ (1.5 mg, 17.9 μmol) were dissolved in water (1 mL). (1R,8S,9s)- Bicyclo[6.1.0]non-4-yn-9-ylmethyl N-succinimidyl carbonate (4 mg, 13.7 mmol) was added and the reaction stirred for 16 h. The solution was dialysed (6000 MWCO) and the resultant solution freeze dried to give the product as a white solid (19 mg, 0.38 μmol, 90%)

### Synthesis of GI87

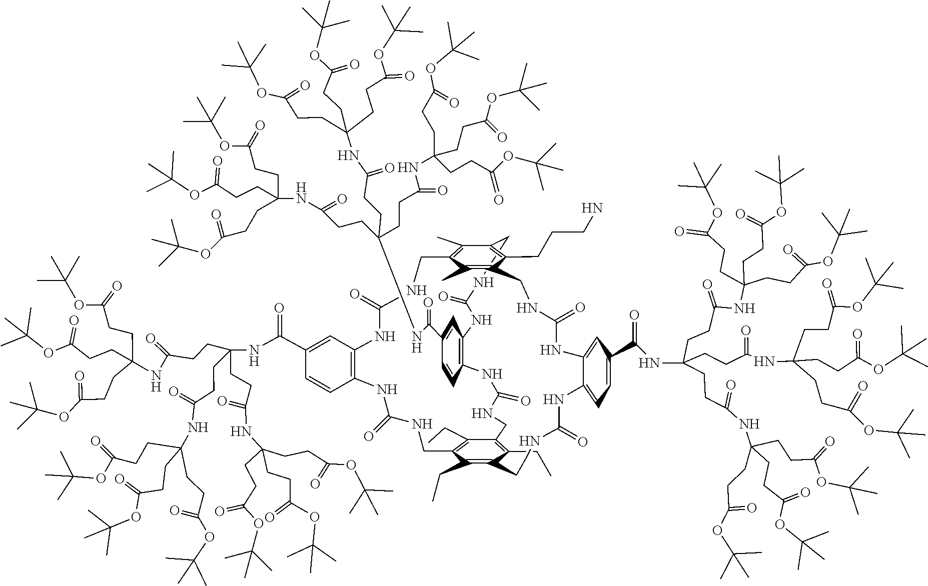

GI47 (20 mg, 3.7 μmol) dissolved in THE (1 mL) and water (0.2 mL), 1 M PMe₃ in THE (0.2 mL, 20 μmol) added and stirred for 18 hours. Solvents removed under a flow of N₂, redissolved in THF and evaporated to dryness again, to give the product as a white solid (19 mg, 3.5 μmol, 96%).

MS (electrospray, +ve) (of sample treated with TFA to hydrolyse ^{t}Bu esters), [M+3H]³⁺ calculated for C₁₇₆H₂₄₃N₂₅O₇₂ requires: 1288.2177, found: 1288.4.

### Synthesis of GI88

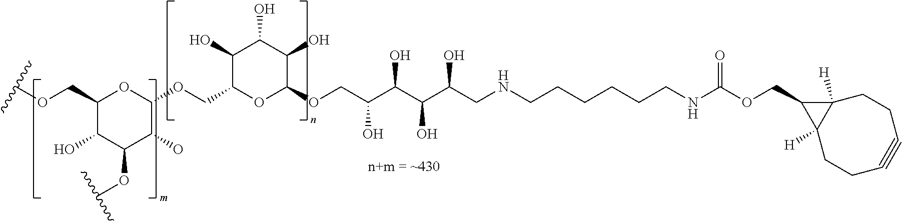

Monoamino dextran MW 70 kDa (40 mg, 0.57 μmol) and NaHCO₃ (1.7 mg, 20 μmol) were dissolved in water (1 mL). (1R,8S,9s)- Bicyclo[6.1.0]non-4-yn-9-ylmethyl N-succinimidyl carbonate (2 mg, 6.9 μmol) was added and the reaction stirred for 5 h. The reaction mixture was transferred to a Vivaspin centrifugal concentrator (30 kDa MWCO, PES). Sample was washed exhaustively with water before freeze drying to give the product as a white solid (28 mg, 0.46 μmol, 80%).

### Synthesis of GI89

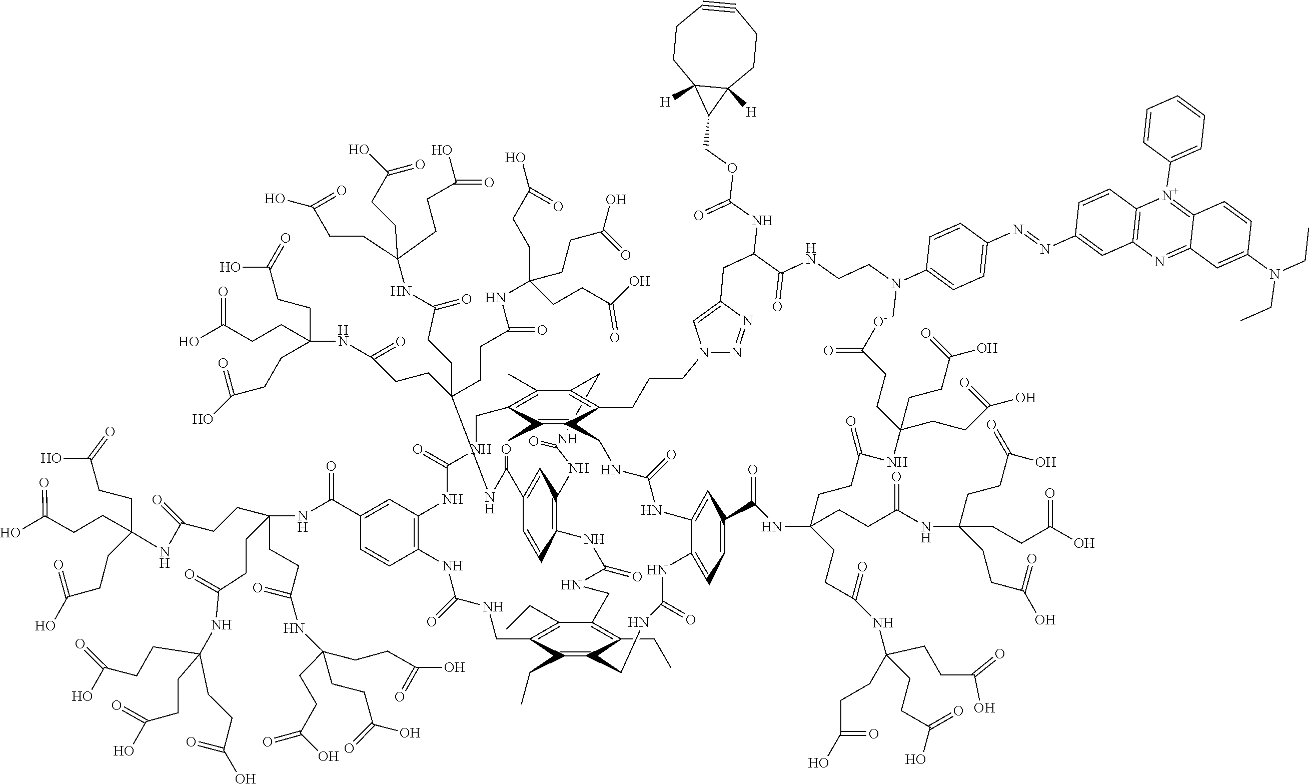

Prepared according to General Procedure C from GI90 (3 mg, 0.66 μmol), (1R,8S,9s)-bicyclo[6.1.0]non-4-yn-9-ylmethyl N-succinimidyl carbonate (1.2 mg, 4.4 μmol), added in two equal portions 8 hours apart, and NaHCO₃ (2 mg, 23.8 μmol) in MeCN (0.5 mL) and water (0.5 mL). Purified by RP MPLC (H₂O with increasing MeCN (0.1% formic acid)). Product obtained as a blue solid (2 mg, 0.43 μmol, 65%).

MS (electrospray, +ve), [M+3H]³⁺ calculated for C₂₂₃H₂₉₆N₃₅O₇₅ requires: 1555.3492, found: 1555.5.

### Synthesis of GI90

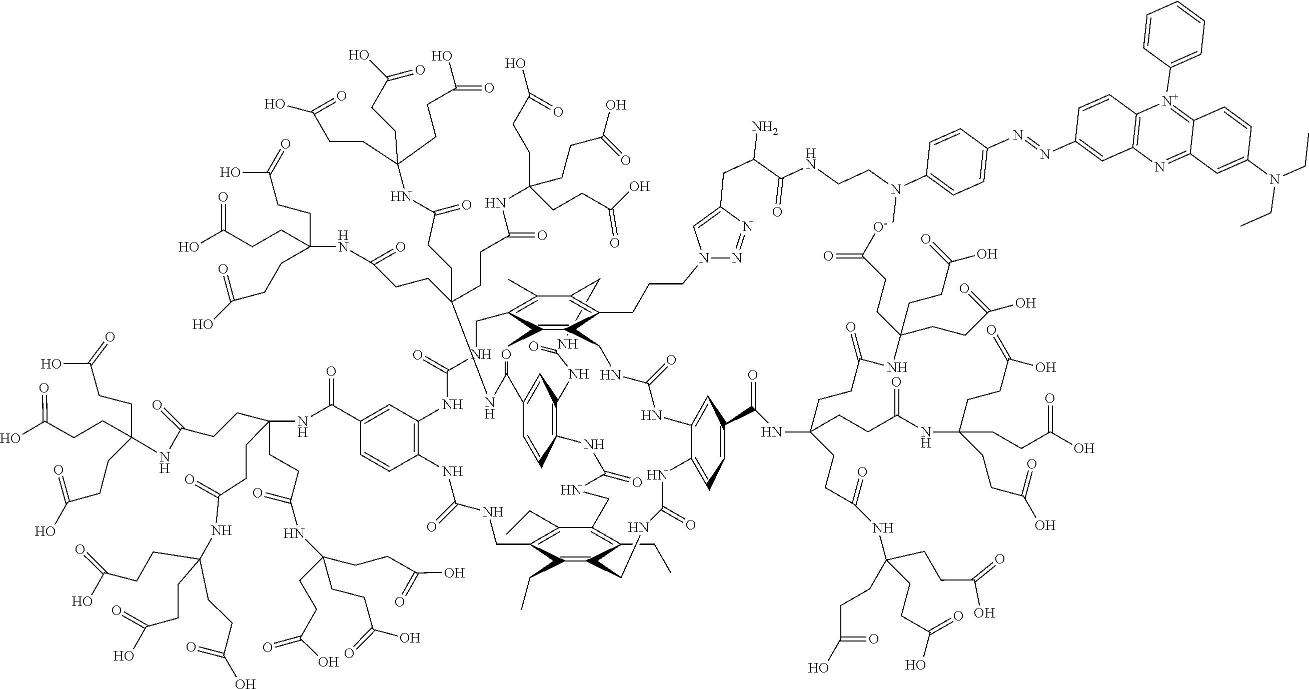

Prepared according to General Procedure D from GI91 (11 mg, 1.8 μmol) in TFA (0.5 mL) and DCM (1 mL). Product obtained as a blue solid (3 mg, 0.7 μmol, 39%).

MS (electrospray, +ve), [M+3H]³⁺ calculated for C₂₁₂H₂₈₄N₃₅O₇₃ requires: 1496.6546, found: 1496.8.

### Synthesis of GI91

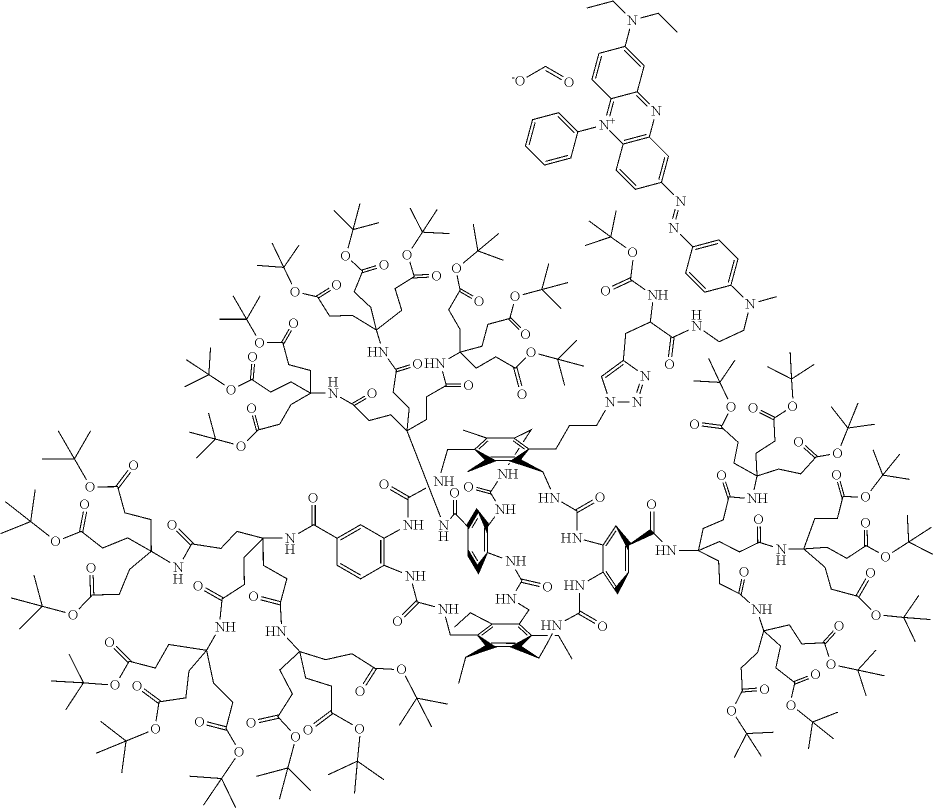

Prepared according to General Procedure B from GI47 (10 mg, 1.9 μmol), G192 (2.2 mg, 3.1 μmol), CuSO₄·5H₂O (0.5 mg, 2.0 μmol) and sodium ascorbate (0.5 mg, 2.5 μmol) in water (0.6 mL) and THF (1.2 mL). Purified by RP MPLC (H₂O with increasing acetone). Product obtained as a blue solid (11 mg, 1.8 μmol, 95%).

MS (electrospray, +ve), [M+3H]³⁺ calculated for C₂₀₃H₂₆₃N₃₃O₇₂Ru requires: 1494.5806, found: 1494.4.

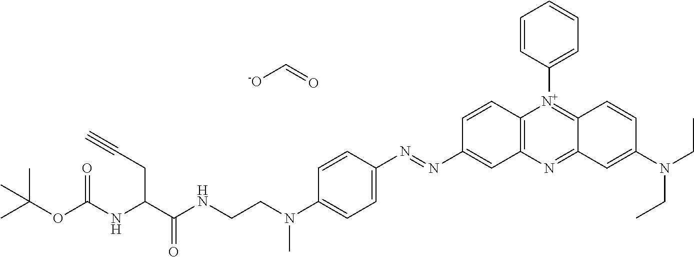

Boc-L-2-propargylglycine (2 mg, 9.4 μmol) and HOBt (1 mg, 6.5 μmol) dissolved in THF (1 mL) with DIPEA (0.01 mL, 0.052 mmol). DCC (2 mg, 9.7 μmol) added and stirred for 35 min. G193 (5 mg, 9.9 μmol) added as a solution in DMF (0.2 mL) and the reaction stirred for 16 h. Solvents removed under reduced pressure and the residue purified by RP MPLC (H₂O with increasing MeCN (0.1% formic acid)). Product obtained as a blue solid (5 mg, 7.1 μmol, 72%).

MS (electrospray, +ve), [M]⁺ calculated for C₄₁H₄₇N₈O₃ requires: 699.3766, found: 699.4.

### Synthesis of GI93

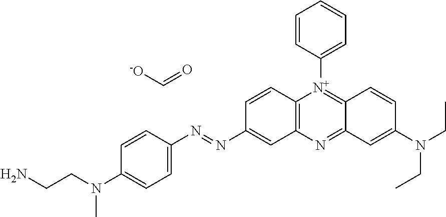

GI94 (50 mg, 94 μmol) and PPh₃ (57 mg, 0.215 mmol) were dissolved in THF (3 mL) and water (3 mL) and stirred for 24 h. THF removed under reduced pressure and remnants purified by RP MPLC (H₂O with increasing acetone). Product obtained as a blue solid (28 mg, 55.5 μmol, 59%).

MS (electrospray, +ve), [M]⁺ calculated for C₃₁H₃₄N₇ requires: 504.2871, found: 504.3.

### Synthesis of GI94

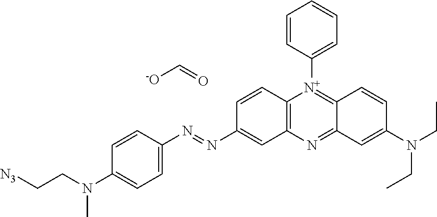

As prepared in Chem. Eur. J., 19: 1686-1699 Fluorescent inhibitor molecules (FIM) procedures

### Example 45: synthesis FIM1

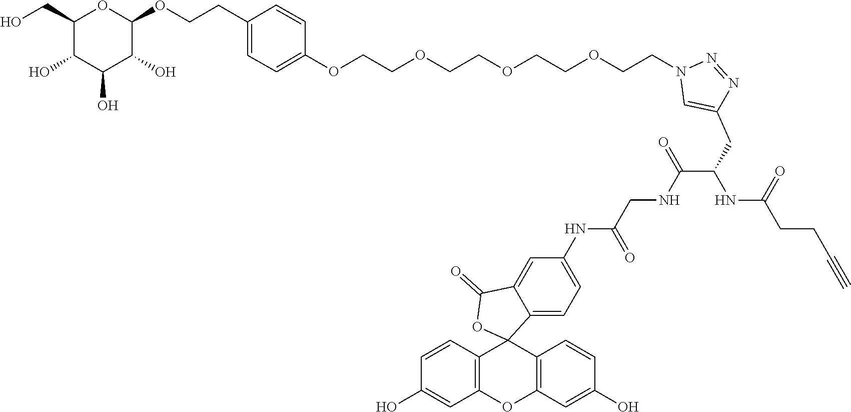 [SMILES: C#CCCC(=O)N[C@@H](Cc1cn(CCOCCOCCOCCOc2ccc(CCO[C@@H]3O[C@H](CO)[C@@H](O)[C@H](O)[C@H]3O)cc2)nn1)C(=O)NCC(=O)Nc1ccc2c(c1)C(=O)OC21c2ccc(O)cc2Oc2cc(O)ccc21]

Prepared according to General Procedure A from pentynoic acid (1.5 mg, 0.015 mmol), 149 (5.0 mg, 0.005 mmol), HBTU (2.3 mg, 0.006 mmol), HOBt·H₂O (1.5 mg, 0.010 mmol) and DIPEA (3.0 μL, 0.010 mmol) in DMF (0.35 mL). Purified by RP MPLC (H₂O with increasing MeCN). Product obtained as an orange solid (4.0 mg, 0.004 mmol, 74%).

MS (electrospray, +ve) [M+H]⁺ calculated for C₅₄H₆₁N₆O₁₈ requires: 1081.4037, found: 1081.3.

### Example 46: Synthesis of FIM2

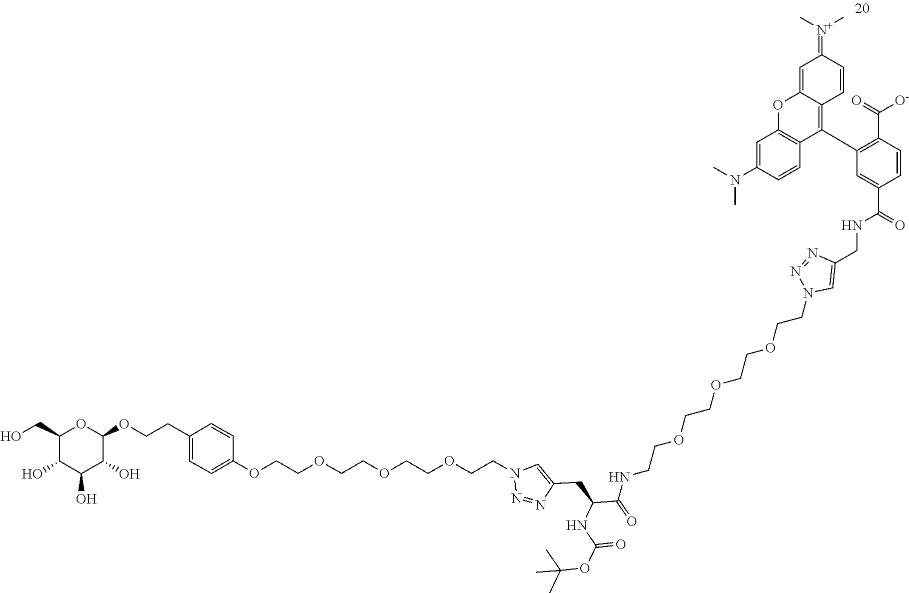 [SMILES: CN(C)c1ccc2c(-c3cc(C(=O)NCc4cn(CCOCCOCCOCCNC(=O)[C@H](Cc5cn(CCOCCOCCOCCOc6ccc(CCO[C@@H]7O[C@H](CO)[C@@H](O)[C@H](O)[C@H]7O)cc6)nn5)NC(=O)OC(C)(C)C)nn4)ccc3C(=O)[O-])c3ccc(=[N+](C)C)cc-3oc2c1]

Prepared according to General Procedure B from I71 (13 mg, 0.014 mmol), 5-TAMRA alkyne (8.0 mg, 0.017 mmol), CuSO₄·5H₂O (3.2 mg, 0.013 mmol), sodium ascorbate (2.8 mg, 0.014 mmol) in THF (0.25 mL) and water (0.25 mL). Purified by RP MPLC (H₂O with increasing MeCN). Product obtained as a violet solid (5.4 mg, 0.005 mmol, 99%).

MS (electrospray, +ve) [M+H]⁺ calculated for C₆₈H₉₂N₁₁O₂₀ requires: 1382.6515, found: 1382.6.

### Example 47: Synthesis of FIM3

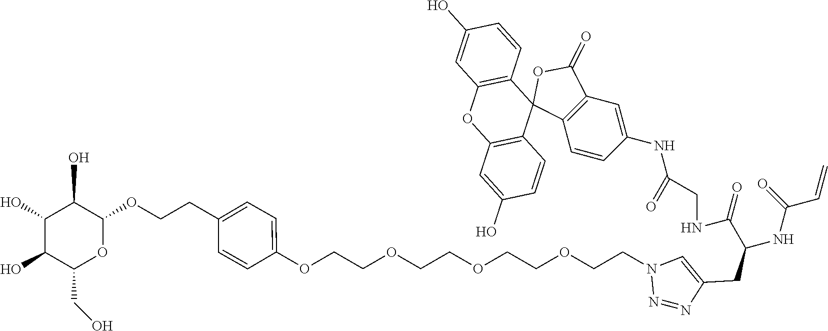 [SMILES: C=CC(=O)N[C@@H](Cc1cn(CCOCCOCCOCCOc2ccc(CCO[C@@H]3O[C@H](CO)[C@@H](O)[C@H](O)[C@H]3O)cc2)nn1)C(=O)NCC(=O)Nc1ccc2c(c1)C(=O)OC21c2ccc(O)cc2Oc2cc(O)ccc21]

Prepared according to General Procedure B from I18 (2.6 mg, 0.005 mmol), I51 (3.2 mg, 0.005 mmol), CuSO₄·5H₂O (1.1 mg, 0.004 mmol), sodium ascorbate (2.9 mg, 0.014 mmol) in THF (0.25 mL) and water (0.25 mL). Purified by RP MPLC (H₂O with increasing MeCN (0.1% formic acid)). Product obtained as an orange solid (5.4 mg, 0.005 mmol, 99%).

MS (electrospray, +ve) [M]⁺ calculated for C₅₅H₆₇N₈O₁₆ requires: 1095.4670, found: 1095.4.

### Example 48: Synthesis of FIM4

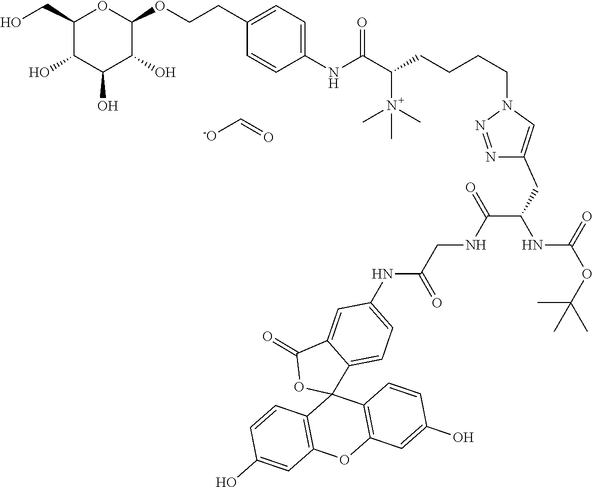

I49 (27 mg, 0.027 mmol) and NaHCO₃ (63 mg, 3.393 mmol) were solubilized in H₂O (6 mL) and MeCN (4 mL). Then, the acryloyl chloride (8 mg, 0.088) was added as a solution in MeCN. After 1 hour, 9 drops of 0.2 M Na₂CO₃ were added to the reaction mixture to hydrolyse the ester by-products. The reaction's pH was 9-10. Added 3 mL of 1 M HCl and pH was found to be 7. The Product was purified by RP MPLC (H₂O with increasing MeCN (0.1% formic acid)). Fractions collected and freeze-dried to give product (17 mg, 0.016 mmol) as a solid.

MS (electrospray, +ve), [M+H]⁺ calculated for C₅₂H₅₈N₆O₁₃ requires: 1055.3881, found: 1055.3.

### Example 49: Synthesis of FIM5

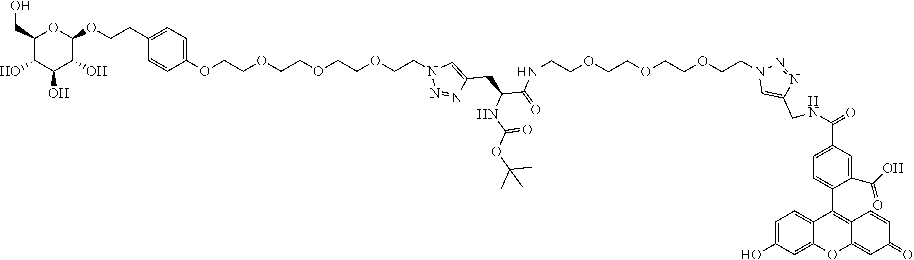 [SMILES: CC(C)(C)OC(=O)N[C@@H](Cc1cn(CCOCCOCCOCCOc2ccc(CCO[C@@H]3O[C@H](CO)[C@@H](O)[C@H](O)[C@H]3O)cc2)nn1)C(=O)NCCOCCOCCOCCn1cc(CNC(=O)c2ccc(-c3c4ccc(=O)cc-4oc4cc(O)ccc34)c(C(=O)O)c2)nn1]

To a solution of I71 (21.2 mg, 0.023 mmol), 5-FAM alkyne (6.8 mg, 0.016 mmol) and sodium ascorbate (2.9 mg, 0.015 mmol) in degassed THF (3 mL) and water (2 mL) was added CuSO₄·5H₂O (4.1 mg, 0.016 mmol) as a solution in water (0.5 mL). The reaction mixture was diluted with water and purified by RP MPLC (H₂O with increasing MeCN (0.1% formic acid)) followed by MPLC (DCM with increasing MeOH). After evaporation of the solvent the product was isolated as an orange foam (22 mg, 0.016 mmol, 99%).

MS (electrospray, +ve) [M+H]⁺ calculated for C₆₄H₈₁N₉O₂₂ requires: 1328.5569, found: 1328.5

### Example 50: Synthesis of FIM6

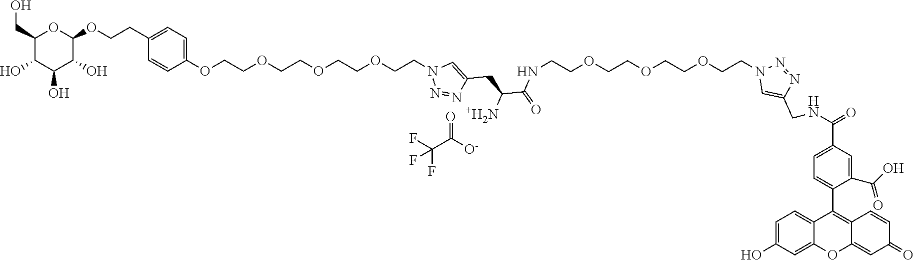 [SMILES: [NH3+][C@@H](Cc1cn(CCOCCOCCOCCOc2ccc(CCO[C@@H]3O[C@H](CO)[C@@H](O)[C@H](O)[C@H]3O)cc2)nn1)C(=O)NCCOCCOCCOCCn1cc(CNC(=O)c2ccc(-c3c4ccc(=O)cc-4oc4cc(O)ccc34)c(C(=O)O)c2)nn1 ; O=C([O-])C(F)(F)F]

FIM5 (21.3 mg, 0.016 mmol) was dissolved in a mixture of TFA (0.25 mL) and DCM (0.25 mL). After 30 minutes the reaction mixture was evaporated then purified by RP MPLC (H₂O with increasing MeCN (0.1% formic acid)). The product was isolated by freeze drying to give a pale orange solid (12 mg, 0.0098 mmol, 61%).

### Example 51: Synthesis of FIM7

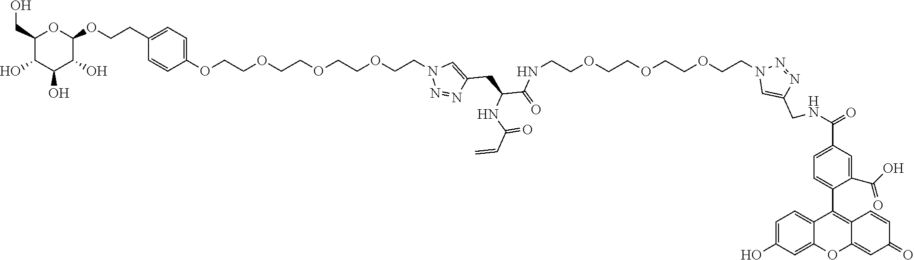 [SMILES: C=CC(=O)N[C@@H](Cc1cn(CCOCCOCCOCCOc2ccc(CCO[C@@H]3O[C@H](CO)[C@@H](O)[C@H](O)[C@H]3O)cc2)nn1)C(=O)NCCOCCOCCOCCn1cc(CNC(=O)c2ccc(-c3c4ccc(=O)cc-4oc4cc(O)ccc34)c(C(=O)O)c2)nn1]

FIM6 (3.0 mg, 2.4 μmol) and NaHCO₃ (5.7 mg, 68 μmol) were dissolved in a mixture of MeCN and water (1:1, 1.6 mL). To this was added a solution of acryloyl chloride (1.3 mg, 14 μmol) in MeCN (0.1 mL). After 30 minutes sodium carbonate was added to adjust to pH 10-11, then after 10 minutes 1 M HCl was added to adjust back to neutral. The solution was purified by RP MPLC (H₂O with increasing MeCN (0.1% formic acid)). After freeze drying the product was isolated as an orange solid (2.5 mg, 1.9 μmol).

MS (electrospray, +ve) [M+H]⁺ calculated for C₆₂H₇₆N₉O₂₁ requires: 1282.5150, found: 1282.4

### Example 52: Synthesis of FIM8

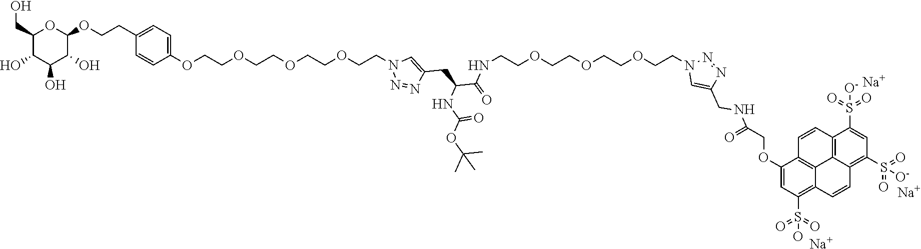 [SMILES: CC(C)(C)OC(=O)N[C@@H](Cc1cn(CCOCCOCCOCCOc2ccc(CCO[C@@H]3O[C@H](CO)[C@@H](O)[C@H](O)[C@H]3O)cc2)nn1)C(=O)NCCOCCOCCOCCn1cc(CNC(=O)COc2cc(S(=O)(=O)O)c3ccc4c(S(=O)(=O)[O-])cc(S(=O)(=O)[O-])c5ccc2c3c54)nn1 ; [Na+] ; [Na+] ; [Na+]]

Prepared according to General Procedure B from I71(22 mg, 0.024 mmol), “Pyranine_alkyne” (see “Other compounds” section below) (7.0 mg, 0.011 mmol), CuSO₄·5H₂O (0.8 mg, 0.003 mmol), sodium ascorbate (4.5 mg, 0.023 mmol) in water (2 mL). Purified by RP MPLC (H₂O with increasing MeCN (0.1% formic acid)). Product obtained as a pale green solid (6.0 mg, 0.004 mmol, 35%)

MS (electrospray, +ve), [M+H]⁺ calculated for C₆₁H₇₉N₉Na₃O₂₇S₃ requires: 1468.448, found: 1468.3.

### Example 53: Synthesis of FIM9

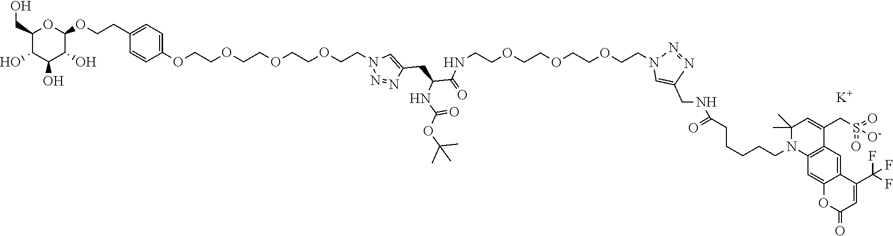 [SMILES: CC(C)(C)OC(=O)N[C@@H](Cc1cn(CCOCCOCCOCCOc2ccc(CCO[C@@H]3O[C@H](CO)[C@@H](O)[C@H](O)[C@H]3O)cc2)nn1)C(=O)NCCOCCOCCOCCn1cc(CNC(=O)CCCCCN2c3cc4oc(=O)cc(C(F)(F)F)c4cc3C(CS(=O)(=O)[O-])=CC2(C)C)nn1 ; [K+]]

Prepared according to General Procedure B from I71 (5.1 mg, 0.005 mmol), AF430-alkyne (1.6 mg, 0.003 mmol), CuSO₄·5H₂O (0.2 mg, 0.001 mmol), sodium ascorbate (1.1 mg, 0.006 mmol) in water (0.12 mL). Purified by RP MPLC (H₂O with increasing MeCN (0.1% formic acid)). Product obtained as an orange solid (3.0 mg, 0.002 mmol, 73%)

MS (electrospray, +ve), [M+H]⁺ calculated for C₆₅H₉₄F₃N₁₀O₂₂S requires: 1455.622, found: 1455.6.

### Example 54: Synthesis of FIM10

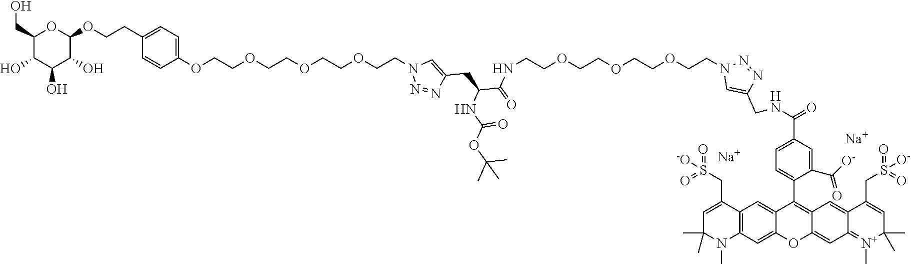

Prepared according to General Procedure B from I71 (2.7 mg, 0.003 mmol), AF594-alkyne (2.0 mg, 0.002 mmol), CuSO₄·5H₂O (0.7 mg, 0.003 mmol), sodium ascorbate (1.5 mg, 0.008 mmol) in water (0.8 mL). Purified by RP MPLC (H₂O with increasing MeCN (0.1% formic acid)). Product obtained as a blue solid (3.8 mg, 0.002 mmol, 99%)

MS (electrospray, +ve), [M+H]⁺ calculated for C₇₃H₁₀₄N₁₁O₂₆S₂ requires: 1674.660, found: 1674.5

### Example 55: Synthesis of FIM11

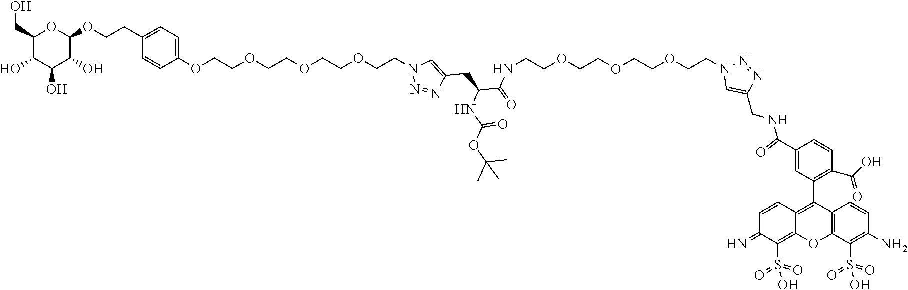 [SMILES: CC(C)(C)OC(=O)N[C@@H](Cc1cn(CCOCCOCCOCCOc2ccc(CCO[C@@H]3O[C@H](CO)[C@@H](O)[C@H](O)[C@H]3O)cc2)nn1)C(=O)NCCOCCOCCOCCn1cc(CNC(=O)c2ccc(C(=O)O)c(-c3c4ccc(=N)c(S(=O)(=O)O)c-4oc4c(S(=O)(=O)O)c(N)ccc34)c2)nn1]

Prepared according to General Procedure B from I71 (4.5 mg, 0.005 mmol), AF488-alkyne (2.0 mg, 0.004 mmol), CuSO₄·5H₂O (1.1 mg, 0.005 mmol), sodium ascorbate (2.5 mg, 0.013 mmol) in water (1.3 mL). Purified by RP MPLC (H₂O with increasing MeCN (0.1% formic acid)). Product obtained as an orange solid (2.9 mg, 0.002 mmol, 56%)

MS (electrospray, +ve), [M+H]⁺ calculated for C₆₄H₈₄N₁₁O₂₆S₂ requires: 1486.503, found: 1486.4

### Example 56: Synthesis of FIM12

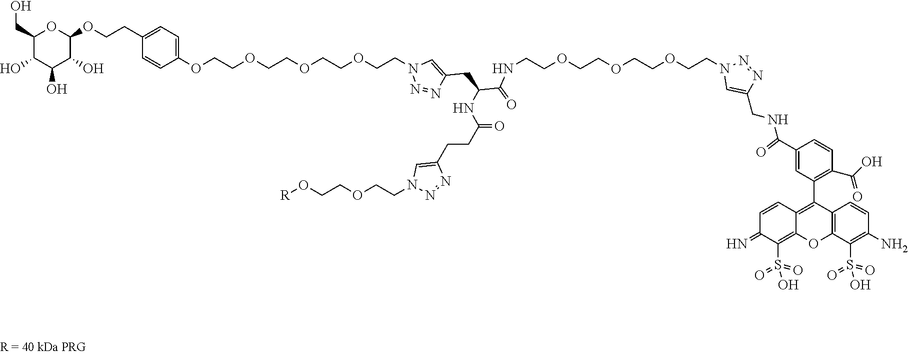

Prepared according to General Procedure B from I17 (2.3 mg, 1.5 μmol), 40 kDa mPEG-N₃ (44 mg, -1.1 μmol), sodium ascorbate (2.2 mg, 11 μmol) and CuSO₄·5H₂O (1.4 mg, 5.5 μmol) in water (1.5 mL) and MeOH (0.01 mL). Reaction solution was dialysed(6K MWCO) tube in water (1.8 L), then the product was freeze dried to give a salmon pink solid (42 mg, 1.01 μmol, 92%).

Absₘₐₓ=488 nm. Emₘₐₓ=520 nm.

### Example 57: Synthesis of FIM13

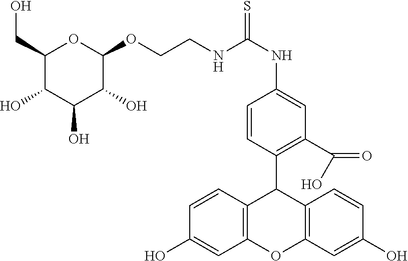 [SMILES: O=C(O)c1cc(NC(=S)NCCO[C@@H]2O[C@H](CO)[C@@H](O)[C@H](O)[C@H]2O)ccc1C1c2ccc(O)cc2Oc2cc(O)ccc21]

Dissolved 2-aminoethyl-beta-D-glucopyranoside (24.5 mg, 0.063 mmol) in DMF (0.7 mL) and added fluorescein isothiocyanate (14.3 mg, 0.064 mmol). Stirred overnight then added water which gave a yellow precipitate and mother liquor. This suspension was purified by RP MPLC (H₂O with increasing MeOH) to give a yellow powder (18 mg, 0.029 mmol, 47%)

¹H NMR (400 MHz, Methanol-d₄) δ 8.16 (s, 1H), 7.81 (dd, J=8.2, 2.0 Hz, 1H), 7.16 (d, J=8.2 Hz, 1H), 6.77 (d, J=8.9 Hz, 2H), 6.72 (d, J=2.4 Hz, 2H), 6.61 (dd, J=8.7, 2.4 Hz, 2H), 4.40 (d, J=7.8 Hz, 1H), 4.09-3.77 (m, 5H), 3.69 (dd, J=12.0, 6.1 Hz, 1H), 3.46-3.35 (m, 2H), 3.28-3.23 (m, 1H).

### Example 58: Synthesis of FIM14

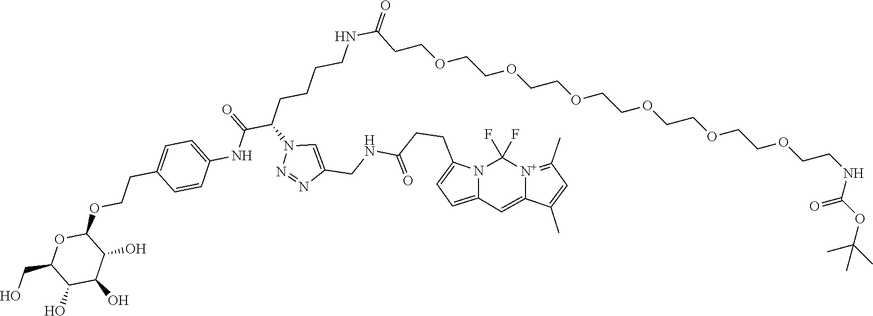 [SMILES: CC1=CC(C)=[N+]2C1=Cc1ccc(CCC(=O)NCc3cn([C@@H](CCCCNC(=O)CCOCCOCCOCCOCCOCCOCCNC(=O)OC(C)(C)C)C(=O)Nc4ccc(CCO[C@@H]5O[C@H](CO)[C@@H](O)[C@H](O)[C@H]5O)cc4)nn3)n1C2(F)F]

Prepared according to General Procedure B from I30 (12 mg, 0.013 mmol), BDP FL alkyne (5,5-difluoro-1,3-dimethyl-7-(3-oxo-3-(prop-2-yn-1-ylamino)propyl)-5H-414-dipyrrolo[1,2-c: 2′,1′-f][1,3,2]diazaborinin-5-uide, commercially available) (5 mg, 0.015 mmol), CuSO₄·5H₂O (1 mg, 0.004 mmol) and sodium ascorbate (8 mg, 0.040 mmol) in THF (1.5 mL) and water (1.5 mL). Purified by RP MPLC (H₂O with increasing MeCN (0.1% formic acid)). Product obtained as a green solid (11.0 mg, 0.009 mmol, 67%).

¹H NMR (400 MHz, Chloroform-d) δ 10.24 (s, 1H), 8.20 (s, 1H), 8.03 (s, 1H), 7.46 (s, 4H), 7.04 (d, J=18.0 Hz, 3H), 6.82 (s, 1H), 6.21 (s, 1H), 6.06 (s, 1H), 5.72 (s, 1H), 5.22 (s, 1H), 4.51 (s, 2H), 4.22 (s, 1H), 3.90 (s, 2H), 3.55 (dd, J=29.9, 11.4 Hz, 30H), 3.27 (s, 6H), 2.66 (s, 6H), 2.48 (s, 6H), 2.18 (s, 5H), 1.42 (s, 16H).

MS (electrospray, +ve), [M+H]⁺ calculated for C₅₇H₈₇BF₂N₉O₁₇ requires: 1218.6276, found: 1218.5.

### Example 59: Synthesis of FIM15

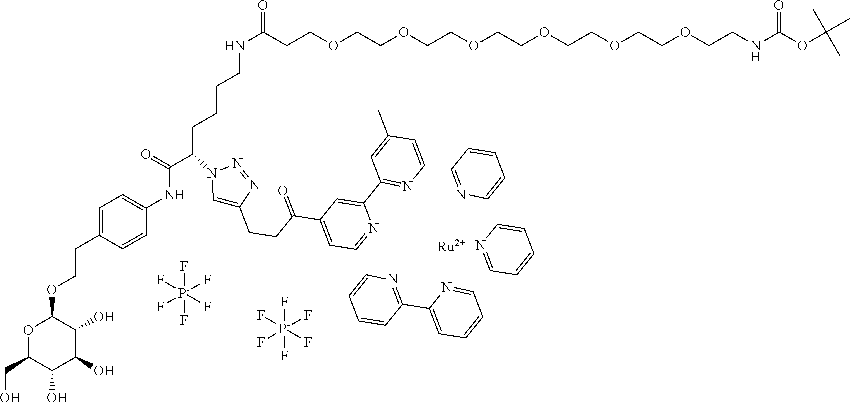

Prepared according to General Procedure B from I30 (3 mg, 3.4 μmol), “Ru(bpy)3 alkyne” (see “Other compounds” section below) (2 mg, 2.1 μmol), CuSO₄·5H₂O (1 mg, 4.0 μmol) and sodium ascorbate (0.5 mg, 2.5 μmol) in THF (0.8 mL) and water (1 mL). Purified by RP MPLC (H₂O with increasing MeCN (0.1% formic acid)). Product obtained as an orange solid (4 mg, 2.2 μmol, 100%).

¹H NMR (400 MHz, Methanol-d₄) δ 8.72-8.60 (m, 2H), 8.10 (d, J=8.0 Hz, 2H), 7.93 (s, 1H), 7.79 (s, 2H), 7.62 (d, J=5.7 Hz, 1H), 7.56-6.93 (m, 8H), 4.35-3.78 (m, 9H), 3.78-3.38 (m, 24H), 3.18 (q, J=7.2 Hz, 4H), 2.89 (s, 2H), 2.58 (d, J=6.5 Hz, 2H), 2.49 (dd, J=11.3, 5.4 Hz, 4H), 1.60-1.20 (m, 13H).

MS (electrospray, +ve), [M]²⁺ calculated for C₇₅H₉₇N₁₃O₁₇Ru requires: 776.8079, found: 776.9.

### Example 60: Synthesis of FIM16

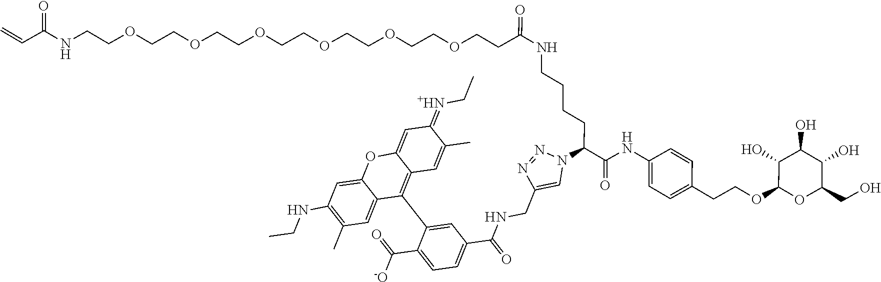

Prepared according to General Procedure C from I22 (3.5 mg, 2.5 μmol), N-acryloxysuccinimide (0.5 mg, 3.0 μmol), NaHCO₃ (1 mg, 12 μmol) in MeCN (0.3 mL) and water (0.3 mL). Purified by RP MPLC (H₂O with increasing MeCN (0.1% formic acid)) to give the product as a pink solid (3 mg, 2.2 μmol, 89%).

MS (electrospray, +ve), [M+H]⁺ calculated for C₆₈H₉₂N₉O₁₉ requires: 1338.6504, found: 776.9.

### Example 61: Synthesis of FIM17

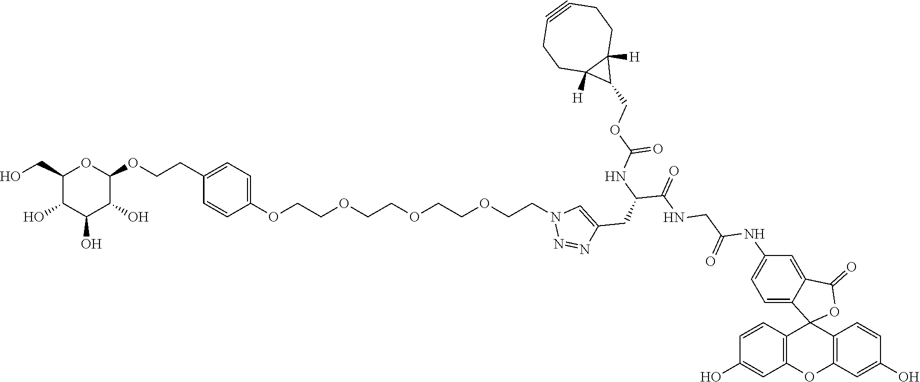 [SMILES: O=C(CNC(=O)[C@H](Cc1cn(CCOCCOCCOCCOc2ccc(CCO[C@@H]3O[C@H](CO)[C@@H](O)[C@H](O)[C@H]3O)cc2)nn1)NC(=O)OC[C@@H]1[C@@H]2CCC#CCC[C@@H]21)Nc1ccc2c(c1)C(=O)OC21c2ccc(O)cc2Oc2cc(O)ccc21]

Prepared according to General Procedure C from I49 (5 mg, 5 μmol), (1R,8S,9s)-Bicyclo[6.1.0]non-4-yn-9-ylmethyl N-succinimidyl carbonate (4 mg, 15 μmol), NaHCO₃ (1 mg, 10 μmol) in MeCN (0.5 mL) and water (0.5 mL). Purified by RP MPLC (H₂O with increasing MeCN (0.1% formic acid)) to give the product as an orange solid (4 mg, 3.7 μmol, 75%).

¹H NMR (400 MHz, Methanol-d₄) δ 8.41 (d, J=1.9 Hz, 1H), 8.22 (s, 1H), 8.06 (d, J=8.3 Hz, 1H), 7.91 (s, 1H), 7.23-7.06 (m, 3H), 6.87-6.73 (m, 2H), 6.67 (d, J=2.4 Hz, 2H), 6.60 (dd, J=8.7, 3.1 Hz, 2H), 6.53 (dt, J=8.7, 2.0 Hz, 2H), 4.55-4.47 (m, 2H), 4.38 (t, J=7.1 Hz, 1H), 4.28 (d, J=7.8 Hz, 1H), 4.19 (d, J=8.1 Hz, 2H), 3.90-3.83 (m, 3H), 3.80 (dd, J=5.7, 3.6 Hz, 2H), 3.75-3.55 (m, 11H), 3.25-3.11 (m, 3H), 2.83 (t, J=7.3 Hz, 2H), 2.26-2.05 (m, 6H), 1.52 (d, J=12.2 Hz, 2H), 1.35 (p, J=8.6 Hz, 1H), 0.90 (d, J=10.6 Hz, 2H).

MS (electrospray, +ve), [M+H]⁺ calculated for C₆₀H₆₉N₆O₁₉ requires: 1177.4612, found: 1177.4.

### Example 62: Synthesis of FIM18

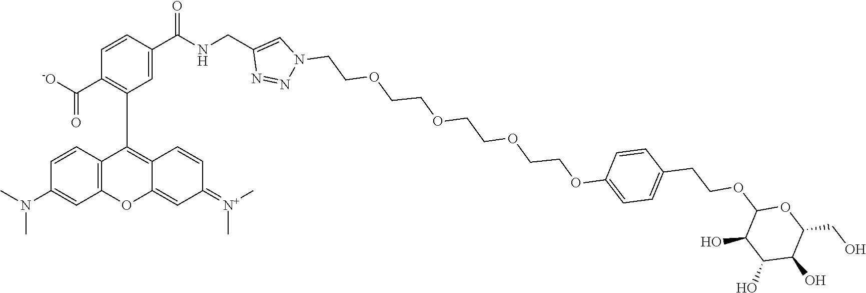

Prepared according to General Procedure B from I24 (10 mg, 0.020 mmol), TAMRA-alkyne (commercially available; CAS: 945928-17-6) (16 mg, 0.034 mmol), CuSO₄·5H₂O (3 mg, 0.012 mmol) and sodium ascorbate (2 mg, 0.008 mmol) in THF (0.5 mL) and water (0.5 mL). Purified by RP MPLC (H₂O with increasing MeCN (0.1% formic acid)). Product obtained as a red solid (7 mg, 6.8 μmol, 34%).

¹H NMR (400 MHz, Deuterium Oxide) δ 6.96-6.04 (m, 13H), 4.46 (dd, J=5.4, 2.6 Hz, 1H), 4.36 (d, J=21.9 Hz, 2H), 4.21 (d, J=7.9 Hz, 1H), 4.15 (dd, J=10.3, 5.3 Hz, 1H), 4.04 (dd, J=10.4, 2.6 Hz, 1H), 3.97 (s, 1H), 3.76 (d, J=22.3 Hz, 6H), 3.51 (d, J=18.2 Hz, 4H), 3.44-3.26 (m, 8H), 3.19 (d, J=8.0 Hz, 2H), 3.05 (t, J=8.6 Hz, 2H), 2.90 (s, 12H).

MS (electrospray, +ve), [M+H]⁺ calculated for C₅₀H₆₁N₆O₁₄ requires: 969.4241, found: 969.4

### Example 63: Synthesis of FIM19

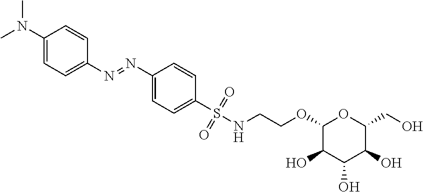 [SMILES: CN(C)c1ccc(/N=N/c2ccc(S(=O)(=O)NCCO[C@@H]3O[C@H](CO)[C@@H](O)[C@H](O)[C@H]3O)cc2)cc1]

I31 (106 mg, 0.156 mmol) dissolved in MeOH (6 mL), MeONa (1 mg, 0.016 mmol) added and stirred for 5 hours. Solvents removed under reduced pressure and residue purified by MPLC (DCM with increasing MeOH) to give the product as a brown solid (79 mg, 0.156 mmol, 100%).

MS (electrospray, +ve), [M+H]⁺ calculated for C₂₂H₃₁N₄O₈S requires: 511.1858, found: 511.2.

### Example 64: Synthesis of FIM20

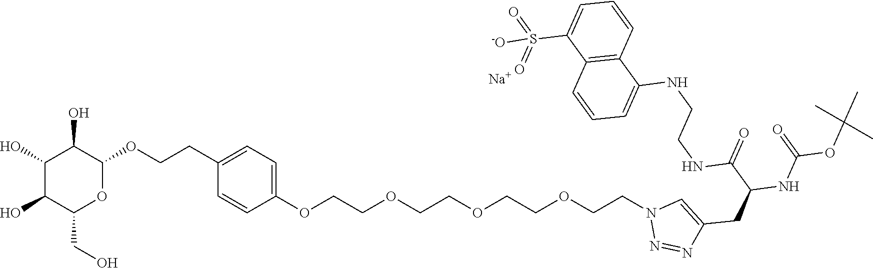

Prepared according to General Procedure A from I70 (10 mg, 0.014 mmol), EDANS (5 mg, 0.017 mmol), HBTU (8 mg, 0.021 mmol), HOBt·H₂O (3.2 mg, 0.021 mmol) and DIPEA (10 μL, 0.056 mmol) in THF (0.3 mL). Purified by RP MPLC (H₂O with increasing MeCN (0.1% formic acid)) to give the product as a brown solid (8 mg, 0.008 mmol, 58%).

¹H NMR (400 MHz, Methanol-d₄) δ 8.72 (d, J=8.8 Hz, 1H), 8.20 (d, J=7.1 Hz, 1H), 8.09 (d, J=8.6 Hz, 1H), 7.82 (s, 1H), 7.59-7.53 (m, 2H), 7.34 (d, J=7.5 Hz, 1H), 7.11 (d, J=8.5 Hz, 2H), 6.77 (d, J=8.6 Hz, 2H), 4.49 (t, J=5.0 Hz, 1H), 4.32 (t, J=4.9 Hz, 2H), 4.26 (d, J=7.8 Hz, 1H), 4.10-3.97 (m, 3H), 3.87-3.78 (m, 3H), 3.75 (dd, J=5.7, 3.4 Hz, 2H), 3.71-3.47 (m, 14H), 3.33 (t, J=9.0 Hz, 1H), 3.28-3.22 (m, 6H), 3.21-3.07 (m, 2H), 2.87-2.80 (m, 2H), 1.38 (s, 9H).

### Example 65: Synthesis of FIM21

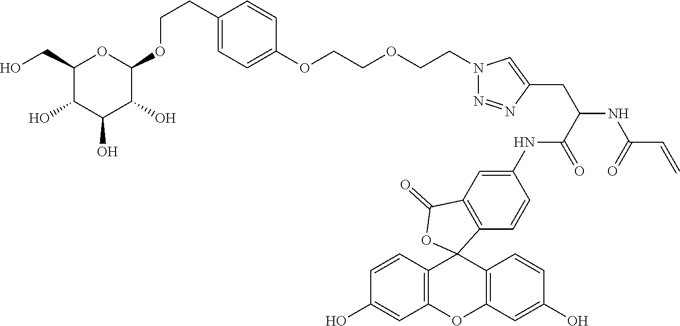 [SMILES: C=CC(=O)NC(Cc1cn(CCOCCOc2ccc(CCO[C@@H]3O[C@H](CO)[C@@H](O)[C@H](O)[C@H]3O)cc2)nn1)C(=O)Nc1ccc2c(c1)C(=O)OC21c2ccc(O)cc2Oc2cc(O)ccc21]

Prepared according to General Procedure C from I13 (23.0 mg, 0.027 mmol), N-acryloxysuccinimide (5.9 mg, 0.035 mmol) and NaHCO₃ (11.3 mg, 0.134 mmol) in MeCN (2 mL) and water (2 mL). Purified by RP MPLC (H₂O with increasing MeCN (0.1% formic acid)). Product obtained as an orange solid (19 mg, 0.021 mmol, 78%)

MS (electrospray, +ve), [M+H]⁺ calculated for C₄₅H₄₆N₅O₁₃ requires: 910.3147, found: 910.3.

### Example 66: Synthesis of FIM22

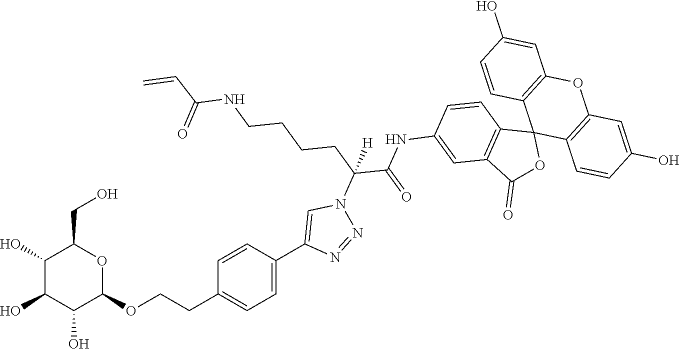 [SMILES: C=CC(=O)NCCCC[C@@H](C(=O)Nc1ccc2c(c1)C(=O)OC21c2ccc(O)cc2Oc2cc(O)ccc21)n1cc(-c2ccc(CCO[C@@H]3O[C@H](CO)[C@@H](O)[C@H](O)[C@H]3O)cc2)nn1]

Prepared according to General Procedure C from I9 (30.0 mg, 0.037 mmol), N-acryloxysuccinimide (8.1 mg, 0.048 mmol) and NaHCO₃ (15.6 mg, 0.185 mmol) in MeCN (2.8 mL) and water (2.8 mL). Purified by RP MPLC (H₂O with increasing MeCN (0.1% formic acid)). Product obtained as an orange solid (15 mg, 0.017 mmol, 47%)

MS (electrospray, +ve), [M+H]⁺ calculated for C₄₅H₄₆N₅O₁₃ requires: 864.3092, found: 864.3

### Example 67; synthesis of FIM23

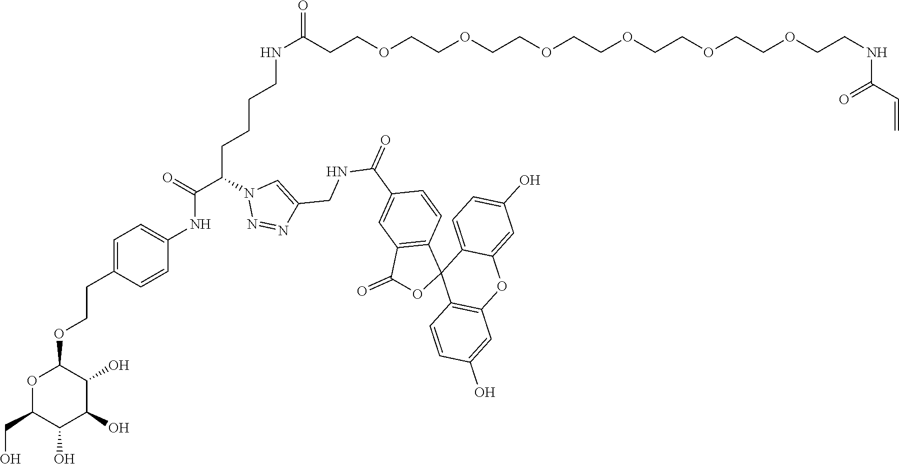 [SMILES: C=CC(=O)NCCOCCOCCOCCOCCOCCOCCC(=O)NCCCC[C@@H](C(=O)Nc1ccc(CCO[C@@H]2O[C@H](CO)[C@@H](O)[C@H](O)[C@H]2O)cc1)n1cc(CNC(=O)c2ccc3c(c2)C(=O)OC32c3ccc(O)cc3Oc3cc(O)ccc32)nn1]

Prepared according to General Procedure C from I11 (10.0 mg, 0.009 mmol), N-acryloxysuccinimide (1.7 mg, 0.010 mmol) and NaHCO₃ (2.1 mg, 0.025 mmol) in MeCN (1.1 mL) and water (1.1 mL). Purified by RP MPLC (H₂O with increasing MeCN (0.1% formic acid)). Product obtained as an orange solid (8 mg, 0.006 mmol, 77%)

MS (electrospray, +ve), [M+H]⁺ calculated for C₆₂H₇₈N₇O₂₁ requires: 1256.525, found: 1256.4

### Example 68: Synthesis of FIM24

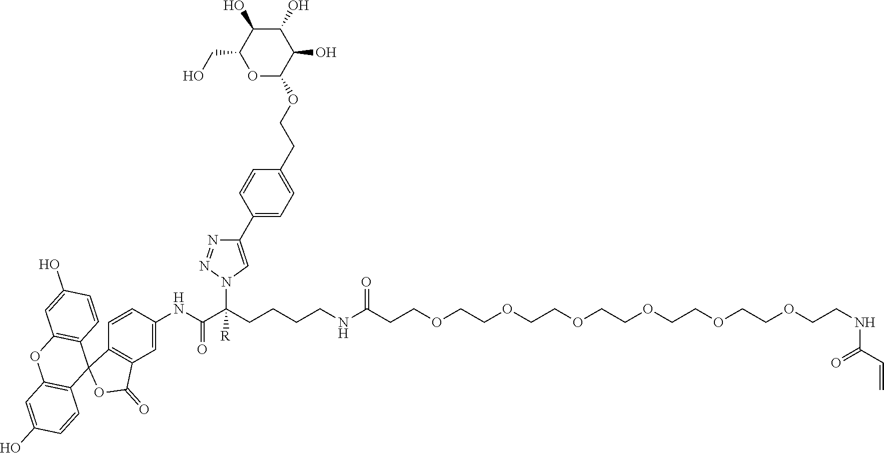

Prepared according to General Procedure C from I7 (10.0 mg, 0.009 mmol), N-acryloxysuccinimide (1.8 mg, 0.010 mmol) and NaHCO₃ (2.2 mg, 0.026 mmol) in MeCN (1 mL) and water (1 mL). Purified by RP MPLC (H₂O with increasing MeCN (0.1% formic acid)). Product obtained as an orange solid (5 mg, 0.004 mmol, 48%)

MS (electrospray, +ve), [M+H]⁺ calculated for C₆₀H₇₂N₆O₂₁ requires: 1199.504, found: 1199.4

### Example 69: Synthesis of FIM25

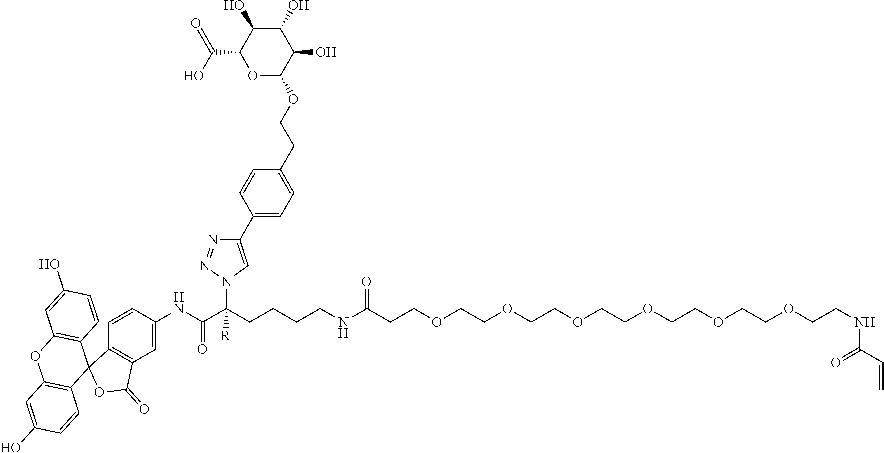

Prepared according to General Procedure C from I3_amine (10.0 mg, 0.009 mmol), N-acryloxysuccinimide (1.8 mg, 0.010 mmol) and NaHCO₃ (2.2 mg, 0.026 mmol) in MeCN (1 mL) and water (1 mL). Purified by RP MPLC (H₂O with increasing MeCN (0.1% formic acid)). Product obtained as an orange solid (10.0 mg, 0.008 mmol, 96%)

MS (electrospray, +ve), [M+H]⁺ calculated for C₆₀H₇₂N₆O₂₁ requires: 1213.475, found: 1213.4.

### Example 70: Synthesis of FIM26

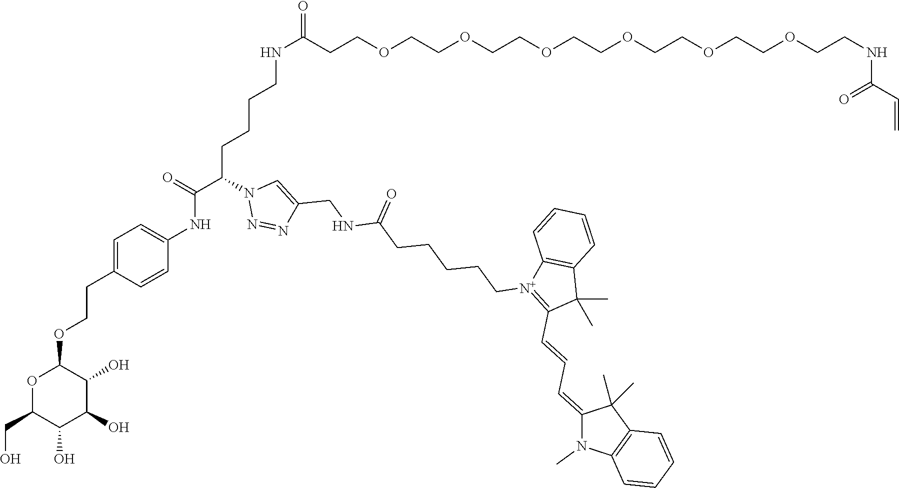 [SMILES: C=CC(=O)NCCOCCOCCOCCOCCOCCOCCC(=O)NCCCC[C@@H](C(=O)Nc1ccc(CCO[C@@H]2O[C@H](CO)[C@@H](O)[C@H](O)[C@H]2O)cc1)n1cc(CNC(=O)CCCCC[N+]2=C(/C=C/C=C3/N(C)c4ccccc4C3(C)C)C(C)(C)c3ccccc32)nn1]

Prepared according to General Procedure C from I2 (10.0 mg, 0.008 mmol), Acryloyl chloride (1 μL, 0.012 mmol) and NaHCO₃ (2.0 mg, 0.012 mmol) in MeCN (1 mL) and water (1 mL). Purified by RP MPLC (H₂O with increasing MeCN (0.1% formic acid)). Product obtained as a pink solid (3.0 mg, 0.002 mmol, 29%)

MS (electrospray, +ve), [M+H]⁺ calculated for C₇₁H₁₀₂N₉O₁₆ requires: 1336.744, found: 1336.6.

### Example 71: Synthesis of FIM27

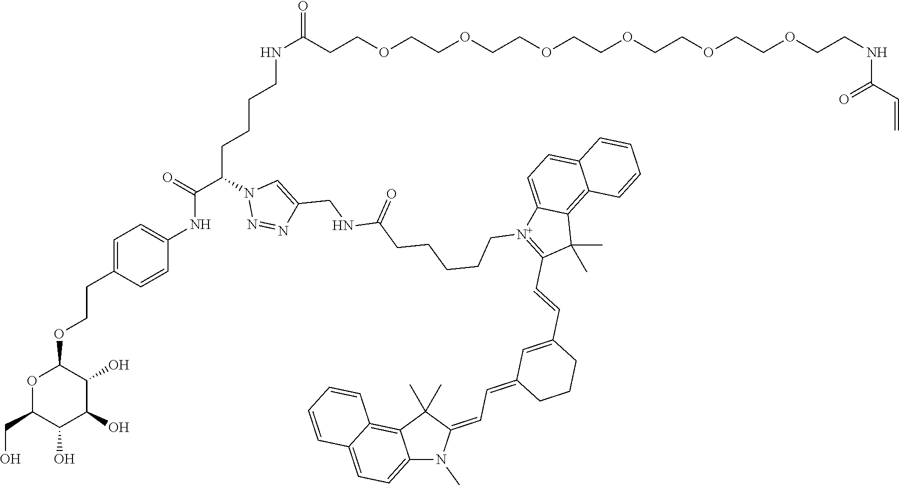 [SMILES: C=CC(=O)NCCOCCOCCOCCOCCOCCOCCC(=O)NCCCC[C@@H](C(=O)Nc1ccc(CCO[C@@H]2O[C@H](CO)[C@@H](O)[C@H](O)[C@H]2O)cc1)n1cc(CNC(=O)CCCCC[N+]2=C(/C=C/C3=CC(=C/C=C4/N(C)c5ccc6ccccc6c5C4(C)C)/CCC3)C(C)(C)c3c2ccc2ccccc32)nn1]

Prepared according to General Procedure C from I1 (6.0 mg, 0.004 mmol), acryloyl chloride (1 μL, 0.012 mmol) and NaHCO₃ (2.0 mg, 0.012 mmol) in MeCN (0.5 mL) and water (0.5 mL). Purified by RP MPLC (H₂O with increasing MeCN (0.1% formic acid)). Product obtained as a green solid (2.0 mg, 0.001 mmol, 32%).

MS (electrospray, +ve), [M+H]⁺ calculated for C₅₆H₁₁₄N₉O₁₆ requires: 1528.838, found: 1528.7

### Example 72: Synthesis of FIM28

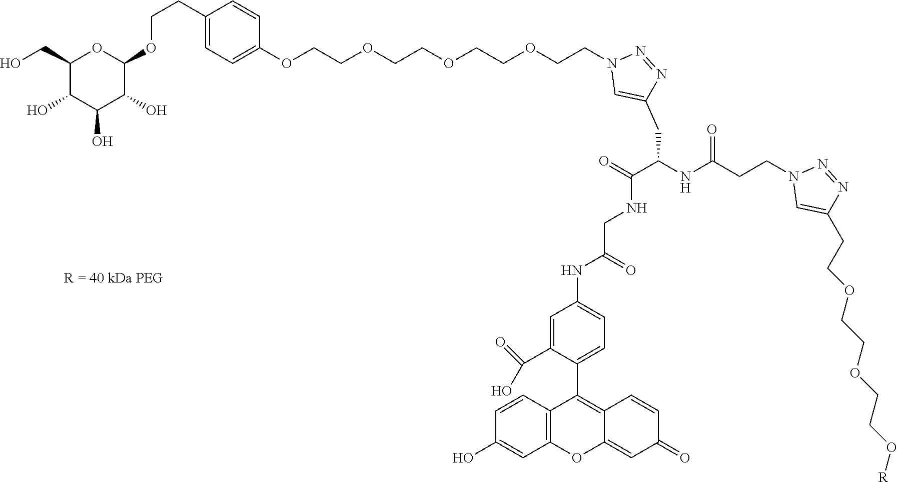

Prepared according to General Procedure B from FIM1 (0.6 mg, 0.6 μmol), 40 kDa mPEG azide (2 mg, 0.5 μmol), CuSO₄·5H₂O (0.6 mg, 2.5 μmol) and sodium ascorbate (2 mg, 10 μmol) in water (0.5 mL). Purified by dialysis (10k MWCO) in water (1.5 L) for 24 hours and freeze dried to give an orange solid (20 mg, 0.6 μmol, 100%).

Absₘₐₓ=480 nm. Emₘₐₓ=520 nm.

### Example 73: Synthesis of FIM29

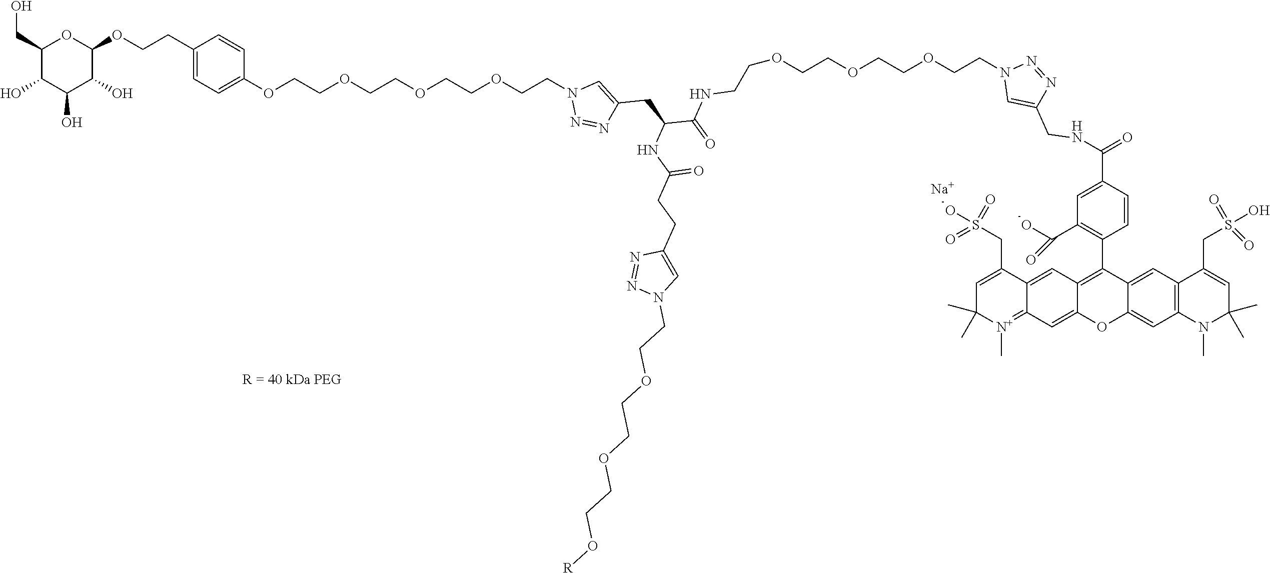

Prepared according to General Procedure B from I68 (1 mg, 0.6 μmol), 40 kDa mPEG azide (2 mg, 0.5 μmol), CuSO₄·5H₂O (0.6 mg, 2.5 μmol) and sodium ascorbate (2 mg, 10 μmol) in water (0.5 mL). Purified by dialysis (10k MWCO) in water (1.5 L) for 24 hours and freeze dried to give a pink solid (18 mg, 0.55 μmol, 90%).

Absₘₐₓ=594 nm. Emₘₐₓ=610 nm.

### Example 74: Synthesis of FIM30

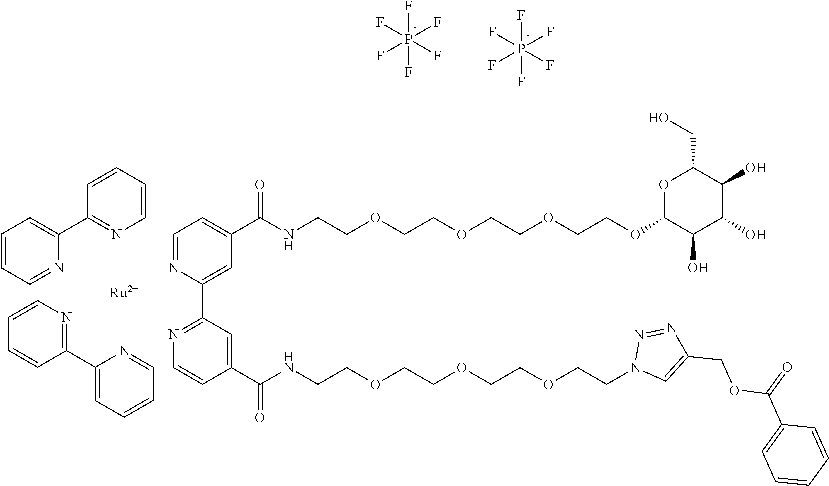

I79 (37.4 mg, 0.040 mmol) and Ru(bipy)₂Cl₂ (17.5 mg, 0.036 mmol) dissolved in EtOH (1.5 mL) and refluxed for 18 hours. EtOH removed under reduced pressure then the crude was dissolved in H₂O (1.0 mL). Ammonium hexafluorophosphate added until a precipitate appeared and stirred for 30 mins. Supernatant decanted and concentrated under reduced pressure. The crude was then loaded onto a bond elute cartridge with AcOH and H₂O, flushed with H₂O (15 mL), then flushed with acetone to elute the desired product. Concentrated under reduced pressure to yield product (48.9 mg, 0.030 mmol, 82.3%).

¹H NMR (400 MHz, Methanol-d₄) δ 9.09-9.04 (m, 2H), 8.69-8.62 (m, 4H), 8.09 (dddd, J=9.6, 7.7, 5.2, 3.7 Hz, 5H), 7.97-7.91 (m, 4H), 7.83-7.74 (m, 6H), 7.59-7.53 (m, 1H), 7.51-7.37 (m, 7H), 4.48 (dd, J=5.7, 4.3 Hz, 2H), 4.27 (d, J=7.8 Hz, 1H), 3.93 (dt, J=10.6, 4.2 Hz, 1H), 3.86-3.76 (m, 3H), 3.70-3.49 (m, 33H), 3.24 (dd, J=5.2, 1.8 Hz, 2H), 3.15 (d, J=8.9 Hz, 1H).

MS (electrospray, +ve), [M]²⁺ calculated for C₆₄H₇₅N₁₁O₁₆Ru²⁺ requires: 677.7213, found: 677.7.

### Example 75: Synthesis of FIM31

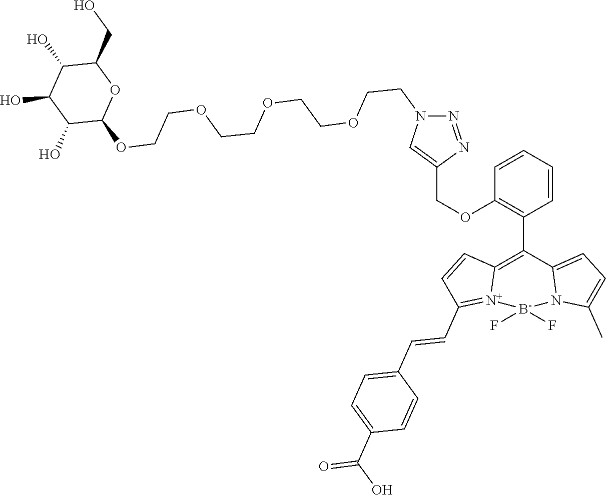 [SMILES: Cc1ccc2n1[B-](F)(F)[N+]1=C(/C=C/c3ccc(C(=O)O)cc3)C=CC1=C2c1ccccc1OCc1cn(CCOCCOCCOCCO[C@@H]2O[C@H](CO)[C@@H](O)[C@H](O)[C@H]2O)nn1]

I75 (5.0 mg, 0.005 mmol) dissolved in 0.5 mL of THF. Then, 0.5 mL of an aqueous solution of LiOH—H₂O (8 mg in 5 mL of H₂O) were added. The reaction was left stirring for 16 hours. The reaction mixture was diluted with 1 M HCl and extracted with DCM (5×15 mL). The combined organic extracts were concentrated under reduced pressure to yield product (2.7 mg, 0.003 mmol, 65%)

MS (electrospray, +ve), [M+H]⁺ calculated for C₄₂H₄₉BF₂N₅O₁₂+requires: 864.3434, found: 864.3.

### Example 76: Synthesis of FIM32

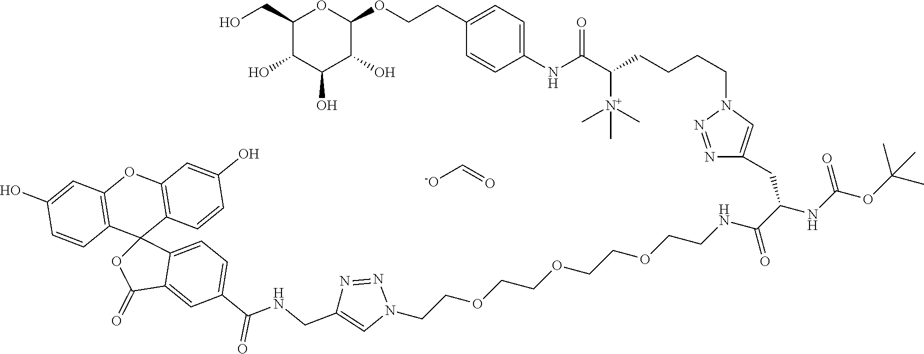

Prepared according to General Procedure B from I18 (3.1 mg, 0.006 mmol), I73 (5.0 mg, 0.006 mmol), CuSO₄·5H₂O (1.3 mg, 0.005 mmol), sodium ascorbate (3.4 mg, 0.017 mmol) in THF (0.30 mL) and water (0.30 mL). Purified by RP MPLC (H₂O with increasing MeCN). Product obtained as an orange solid (2.5 mg, 0.002 mmol, 32%).

MS (electrospray, +ve) [M]⁺ calculated for C₆₅H₈₄N₁₁O₁₉ requires: 1322.5939, found: 1322.6.

### Example 77: Synthesis of FIM33

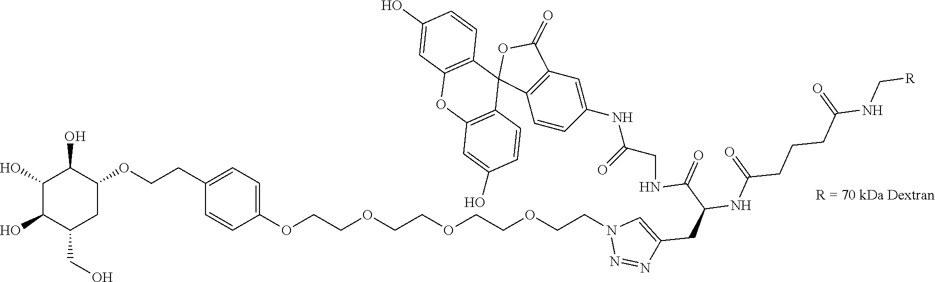

To 161 (0.5 mg, 0.429 μmol) and 70 kDa aminodextran (10.0 mg, 0.143 μmol) was added dry DMSO (0.2 mL). Reaction was sonicated until homogeneous and left to stir at RT. After 22 hours reaction was diluted with water (2.0 mL) and dialysed (6K MWCO) overnight in water (1.8 L) before being transferred to a Vivaspin 6 centrifugal concentrator (30 kDa MWCO, PES). Sample was washed exhaustively with water until filtrate was colourless before freeze drying overnight to give the product as an orange solid (7 mg, 0.098 μmol, 69%).

Absₘₐₓ=480 nm, Emₘₐₓ=530 nm.

### Example 78: Synthesis of FIM34

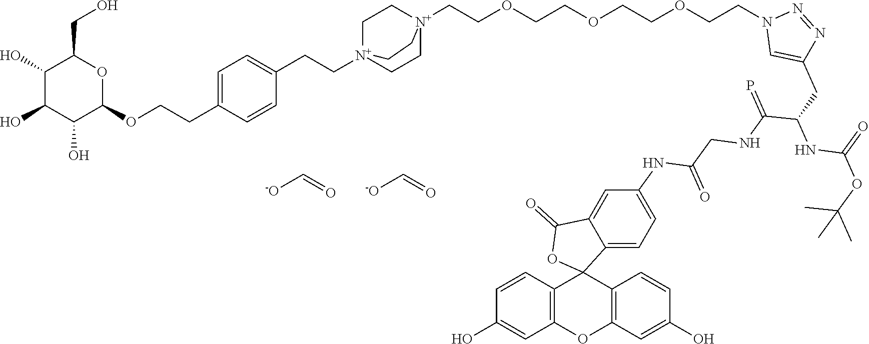

Prepared according to General Procedure B from I37 (2.3 mg, 0.003 mmol), I51 (2.1 mg, 0.004 mmol), CuSO₄·5H₂O (0.7 mg, 0.003 mmol), sodium ascorbate (1.9 mg, 0.010 mmol) in THF (0.20 mL) and water (0.20 mL). Purified by RP MPLC (H₂O with increasing MeCN (0.1% formic acid)). Product obtained as an orange solid (3.5 mg, 0.003 mmol, 83%).

MS (electrospray, +ve) [M]²⁺ calculated for C₆₂H₈₀N₈O₁₈ requires: 612.2790, found: 612.4.

### Example 79: Synthesis of FIM35

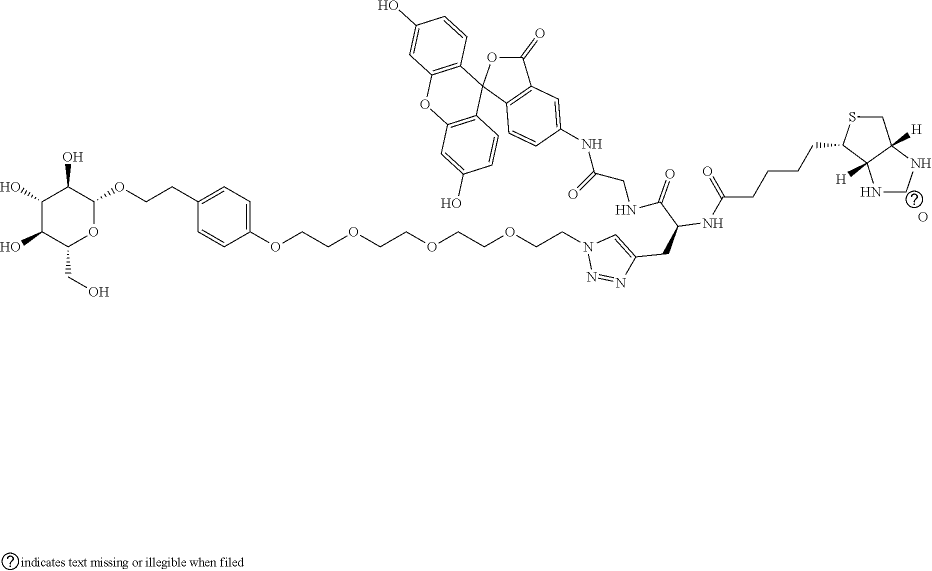

Prepared according to General Procedure C from I49 (3.1 mg, 0.003 mmol), (+)-Biotin-N-hydroxysuccinimide ester (3.2 mg, 0.009 mmol), NaHCO₃ (0.5 mg, 12 μmol) in MeCN (0.6 mL) and water (0.6 mL). Purified by RP MPLC (H₂O with increasing MeCN) to give the product as an orange solid (3.2 mg, 0.003 mmol, 84%).

MS (electrospray, +ve) [M+H]⁺ calculated for C₅₉H₇₁N₈O₁₉S requires: 1227.4551, found: 1227.4.

### Example 80: Synthesis of FIM36

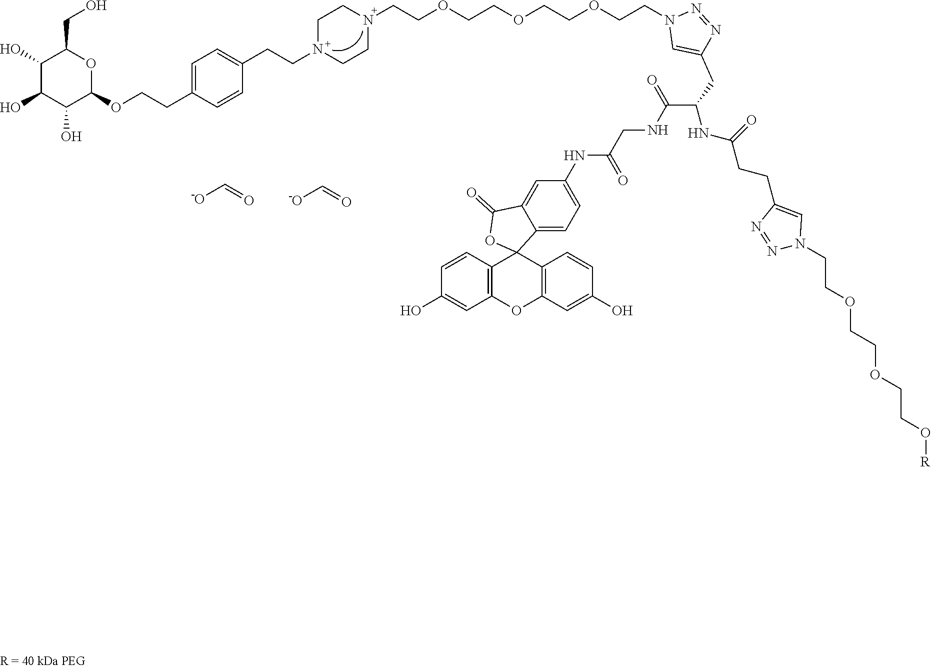

Prepared according to General Procedure B from 40 kDa mPEG-N₃ (20.0 mg, 0.001 mmol), I39 (0.8 mg, 0.001 mmol), CuSO₄·5H₂O (0.6 mg, 0.003 mmol), sodium ascorbate (3.0 mg, 0.015 mmol) in water (1.0 mL). Purified by dialysis (10k MWCO) in water (1.5 L) for 24 hours and freeze dried to give a white solid (15 mg, 0.75 μmol, 75%).

Absₘₐₓ=270 nm, Emₘₐₓ=530 nm.

### Example 81: Synthesis of FIM37

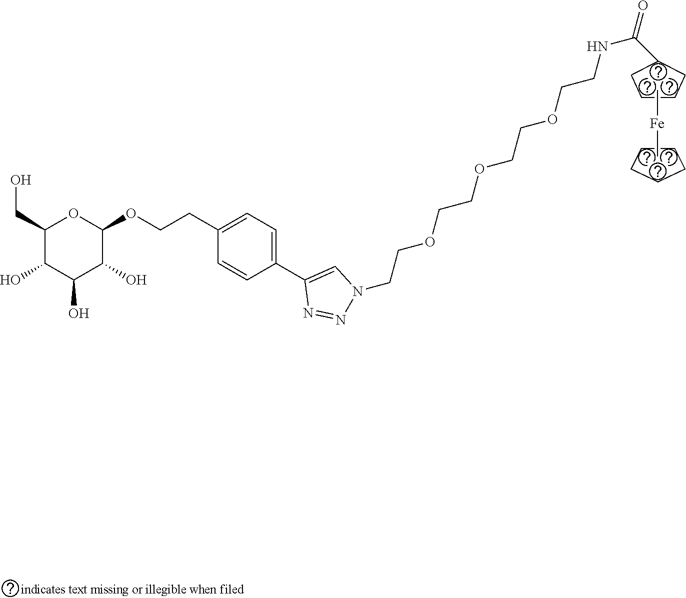

To a solution of the 156 (56 mg, 0.099 mmol) in DMF (2.5 mL) was added ferrocenecarboxylic acid N-succinimidyl ester (39 mg, 0.119 mmol) and 1 drop of DIPEA. After 1.5 hours added a further 3 drops of DIPEA. After 2 h, RM was diluted with H₂O and loaded onto a C₁₈ column. Purified by RP MPLC (H₂O with increasing MeCN), evaporated and freeze dried to give the product as pale brown solid (30 mg, 0.041 mmol, 41%).

MS (electrospray, +ve) [M+H]⁺ calculated for C₃₅H₄₆FeN₄O₁₀ requires: 739.2597, found: 739.3.

### Example 82: Synthesis of FIM38

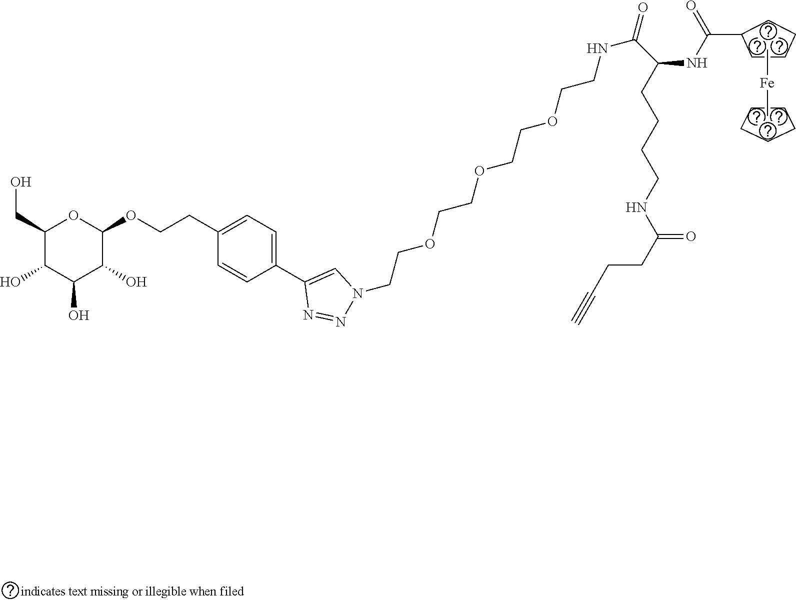

To a solution of I57 (38.6 mg, 44.5 μmol) in water (1 mL) was added sodium bicarbonate (74.8 mg, 890 μmol) followed by MeCN (2 mL), water (2 mL) and 4-pentynoic acid succinimide ester (22.0 mg, 113 μmol). After 90 minutes water (10 mL) was added before reaction mixture was loaded directly onto a C₁₈ column. Purified by RP MPLC (H₂O with increasing MeCN), evaporated and freeze dried to give the product as a pale-yellow solid (23.0 mg, 24.6 μmol, 54%).

¹H NMR (400 MHz, Methanol-d₄) δ 8.32 (s, 1H), 7.73 (d, J=8.3 Hz, 2H), 7.36 (d, J=8.5 Hz, 2H), 4.66-4.58 (m, 2H), 4.48 (dd, J=9.1, 5.5 Hz, 1H), 4.40 (ddd, J=2.2, 1.5, 0.4 Hz, 2H), 4.32 (d, J=7.8 Hz, 1H), 4.21 (s, 5H), 4.12 (dt, J=9.8, 7.2 Hz, 1H), 3.96-3.90 (m, 2H), 3.87 (dd, J=11.7, 1.7 Hz, 1H), 3.79 (dt, J=9.7, 7.1 Hz, 1H), 3.70-3.64 (m, 1H), 3.64-3.56 (m, 4H), 3.56-3.49 (m, 4H), 3.47 (t, J=5.5 Hz, 2H), 3.39-3.32 (m, 3H), 3.28 (dq, J=6.7, 1.6 Hz, 2H), 3.22-3.17 (m, 3H), 2.97 (t, J=7.2 Hz, 2H), 2.44 (tdd, J=6.9, 2.6, 1.0 Hz, 2H), 2.38-2.31 (m, 2H), 2.27 (t, J=2.6 Hz, 1H), 1.90-1.69 (m, 2H), 1.61-1.51 (m, 2H), 1.51-1.36 (m, 2H).

### Example 83: Synthesis of FIM39

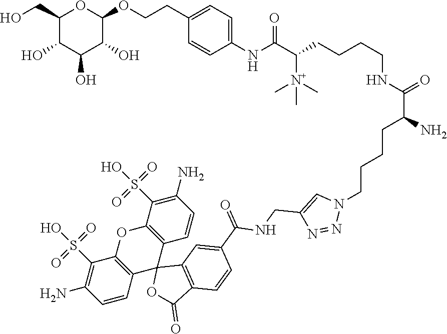

Prepared according to General Procedure B from I65 (1.5 mg, 2.2 μmol), AF488-alkyne (1.0 mg, 1.7 μmol), Cu₂SO₄·5H₂O (0.5 mg, 2.1 μmol), sodium ascorbate (0.5 mg, 2.6 μmol) in water (2 mL). Purified by RP MPLC (H₂O with increasing MeCN (0.1% formic acid)). Product obtained as an orange solid (0.7 mg, 0.54 μmol, 31%).

Absₘₐₓ=488 nm. Emₘₐₓ=520 nm.

MS (electrospray, +ve) [M]⁺ calculated for C₅₃H₆₈N₁₀O₁₈S₂ requires: 1195.4701, found: 1195.4.

### Example 84: Synthesis of FIM40

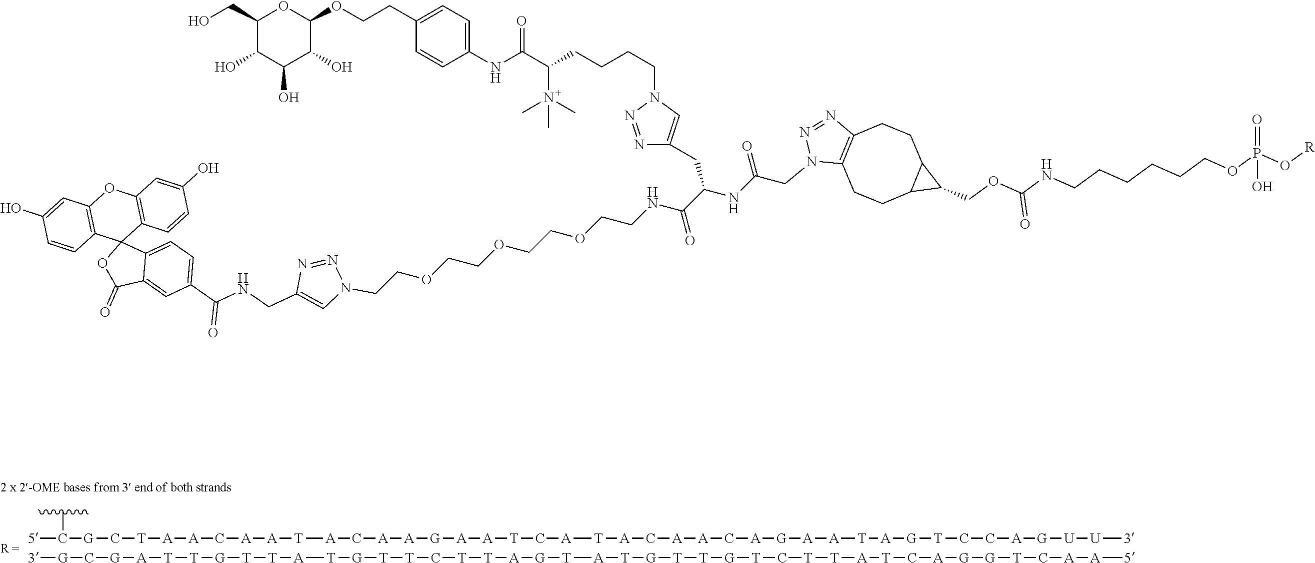

Dissolved (1R,8S,9s)-Bicyclo[6.1.0]non-4-yn-9-ylmethyl N-hexyl carbonate 5′-ssDNA (0.133 mg, 0.01 μmol, sequence: 5′-CGCTAACAATACAAGAATCATACAACAGAATAGTCCAGUU-3′) in pure nuclease free water (0.2 mL, pH 7.4). Added solution of I63 (0.03 mg, 0.02 μmol) in pure nuclease free water (20 μL, pH 7.4) and stirred at RT overnight. Took half of this solution and added to solution of complimentary ssDNA (0.130 mg, 0.01 μmol, sequence: 5;-AACTGGACTATTCTGTTGTATGATTCTTGTATTGTTAGCG-3′) in pure nuclease free water (30 μL). This solution was freeze dried and then redissolved in 10 mM PBS with 1 mM EDTA and heated to 85° C. for 5 minutes and then cooled to RT over 2 hours. The product was purified by centrifugal filtration through a membrane (3k MWCO), rinsing with pure nuclease water (2 mL) 3 times and then freeze dried to give the product (0.2 mg, 0.007 μmol, 75%) as a yellow solid.

Absₘₐₓ=480 nm. Emₘₐₓ=520 nm.

### Example 85: Synthesis of FIM41

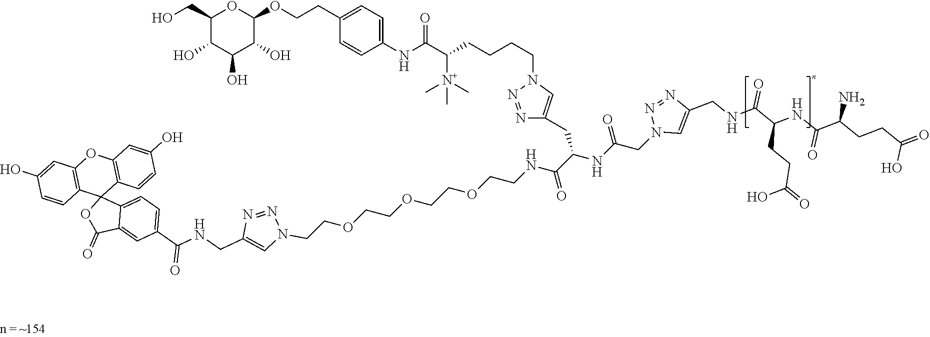

Prepared according to General Procedure B from I63 (0.5 mg, 0.275 μmol), Alkyne-Poly(L-Glu) (154) (5 mg, 0.250 μmol), sodium ascorbate (1 mg, 5.0 μmol), CuSO₄·5H₂O (0.3 mg, 1.3 μmol) and NaHCO₃ (4 mg, 46.3 μmol) in water (0.25 mL) and THF (0.25 mL). Product obtained by dialysis (6k MWCO) of reaction mixture in water (1.8 L), followed by lyophilisation to give an orange powder (3 mg, 1.9 μmol, 56%).

Absₘₐₓ=480 nm. Emₘₐₓ=520 nm.

### Example 86: Synthesis of FIM42

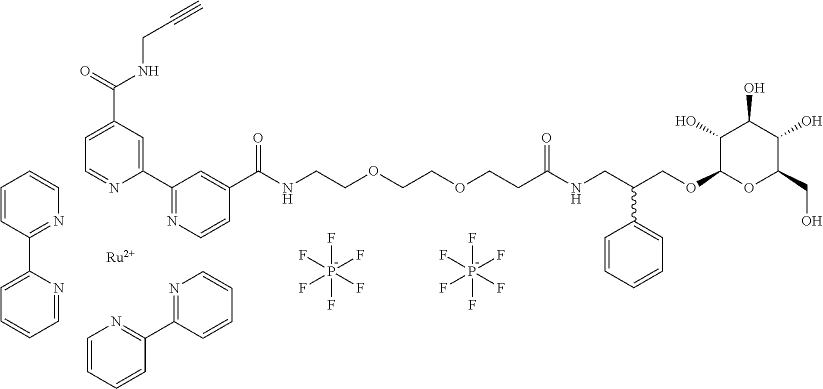

I87 (6.3 mg, 8.6 μmol) and Ru(bipy)₂Cl₂ (3.8 mg, 07.8 μmol) dissolved in EtOH (1.5 mL) and refluxed for 18 hours. EtOH removed under reduced pressure then the crude was dissolved in H₂O (1.0 mL). Ammonium hexafluorophosphate added until a precipitate appeared and stirred for 30 mins. Resultant suspension centrifuged and the pellet then purified by RP MPLC (H₂O with increasing MeCN (0.1% formic acid)) to give the product as a red solid (3.0 mg, 2.1 μmol, 27%).

1H NMR (400 MHz, Methanol-d₄) δ 8.15 (d, J=8.5 Hz, 4H), 7.86-7.77 (m, 8H), 7.51 (d, J=8.5 Hz, 6H), 7.19 (s, 9H), 4.29 (d, J=1.8 Hz, 2H), 4.23 (d, J=2.5 Hz, 2H), 4.04 (s, 2H), 3.84 (s, 2H), 3.71-3.60 (m, 13H), 3.09 (s, 2H), 2.71 (s, 1H), 2.35 (s, 2H).

MS (electrospray, +ve), [M]²⁺ calculated for C₅₇H₆₁N₉O₁₁Ru requires: 574.6762, found: 574.7.

### Example 87: Synthesis of FIM43

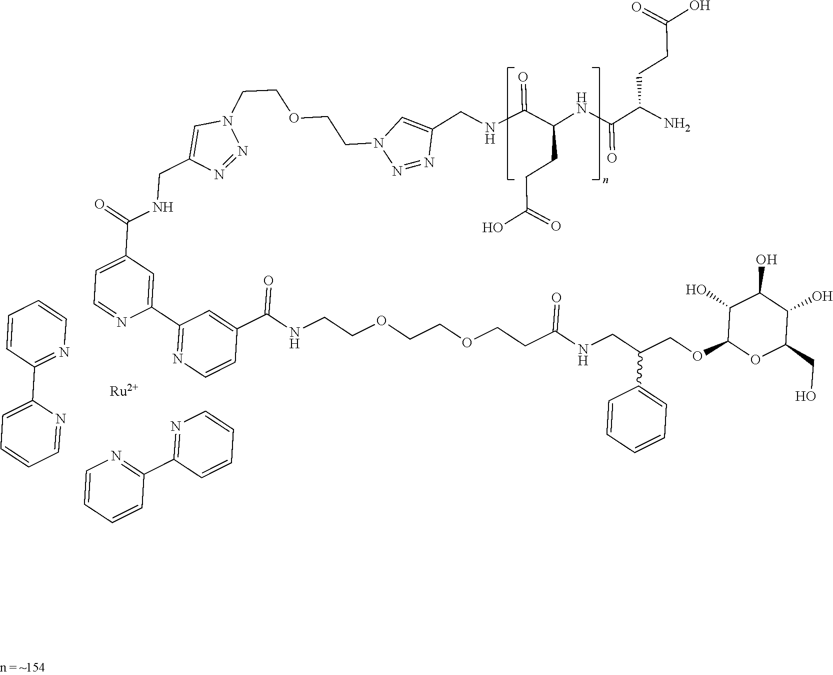

Prepared according to General Procedure B from FIM42 (1.1 mg, 0.774 μmol), Alkyne-Poly(L-Glu) (154) (13 mg, 0.645 μmol), sodium ascorbate (2.5 mg, 12.9 μmol), CuSO₄·5H₂O (0.8 mg, 3.2 μmol) and NaHCO₃ (1.5 mg, 17.4 μmol) in water (1.5 mL). Product obtained by dialysis (6k MWCO) of reaction mixture in water (1.8 L), followed by lyophilisation to give an orange powder (10 mg, 0.463 μmol, 72%).

Absₘₐₓ=450 nm. Emₘₐₓ=660 nm.

### Example 88: Synthesis of FIM44

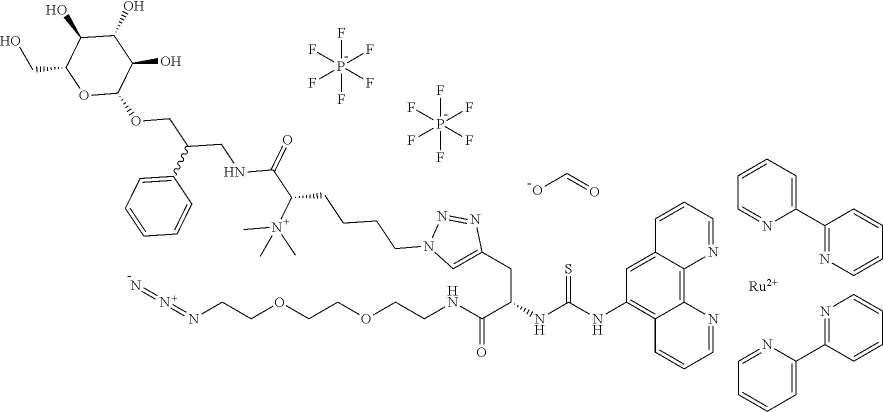

Bis(2,2′-bipyridine)-(5-isothiocyanato-phenanthroline)ruthenium bis(hexafluorophosphate) (1 mg, 1.2 μmol) dissolved in DMF (0.4 mL) and I88 (1.5 mg, 1.8 μmol) added. Reaction stirred for 2 hours then solvents removed under reduced pressure. Residue purified by RP MPLC (H₂O with increasing MeCN (0.1% formic acid)) to give the product as a red solid (0.8 mg, 0.45 μmol, 39%).

MS (electrospray, +ve) [M−2H]⁺ calculated for C₆₈H₈₀N₁₇O₁₀RuS requires: 1428.5033, found: 1428.4.

Absₘₐₓ=450 nm. Emₘₐₓ=620 nm.

### Example 89: Synthesis of FIM45

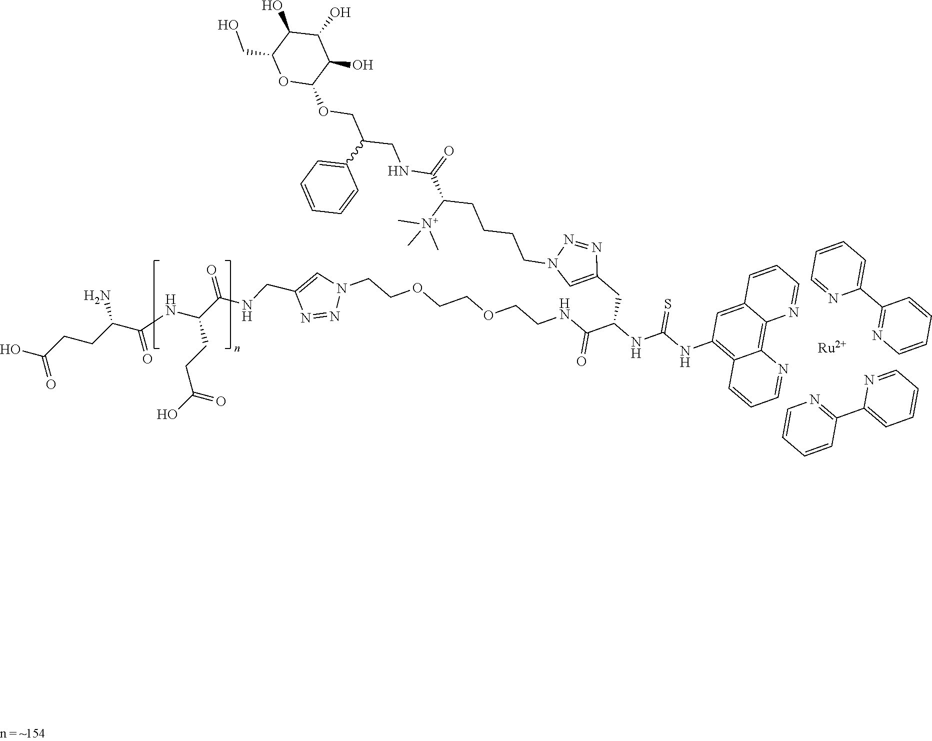

Prepared according to General Procedure B from FIM44 (0.4 mg, 0.220 μmol), Alkyne-Poly(L-Glu) (154) (4 mg, 0.200 μmol), sodium ascorbate (1 mg, 4.0 μmol), CuSO₄·5H₂O (0.2 mg, 1.0 μmol) and NaHCO₃ (3.1 mg, 37.0 μmol) in water (0.5 mL). Product obtained by dialysis (6k MWCO) of reaction mixture in water (1.8 L), followed by lyophilisation to give an orange powder (1.0 mg, 0.463 μmol, 23%).

Absₘₐₓ=450 nm. Emₘₐₓ=620 nm.

### Example 90: Synthesis of FIM46

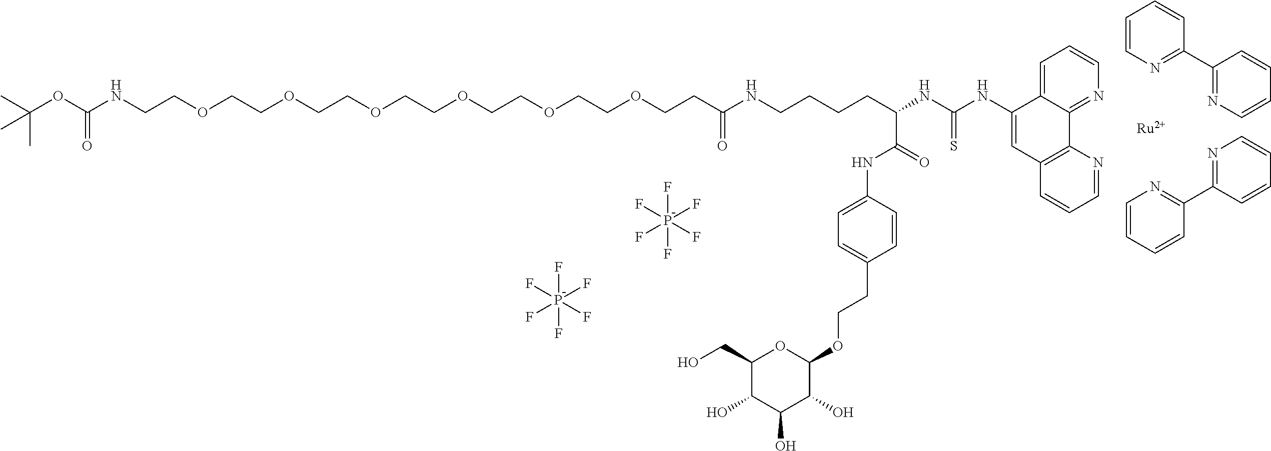

Bis(2,2′-bipyridine)-(5-isothiocyanato-phenanthroline) ruthenium bis(hexafluorophosphate) (1 mg, 1.1 μmol) dissolved in DMF (0.4 mL) and I29 (1.4 mg, 1.6 μmol) added. Reaction stirred for 2 hours then solvents removed under reduced pressure. Residue purified by RP MPLC (H₂O with increasing MeCN (0.1% formic acid)) to give the product as a red solid (1.4 mg, 0.78 μmol, 73%).

MS (electrospray, +ve), [M]²⁺ calculated for C₇₃H₉₃N₁₁O₁₆Ru requires: 756.7778, found: 756.8.

### Example 91: Synthesis of FIM47

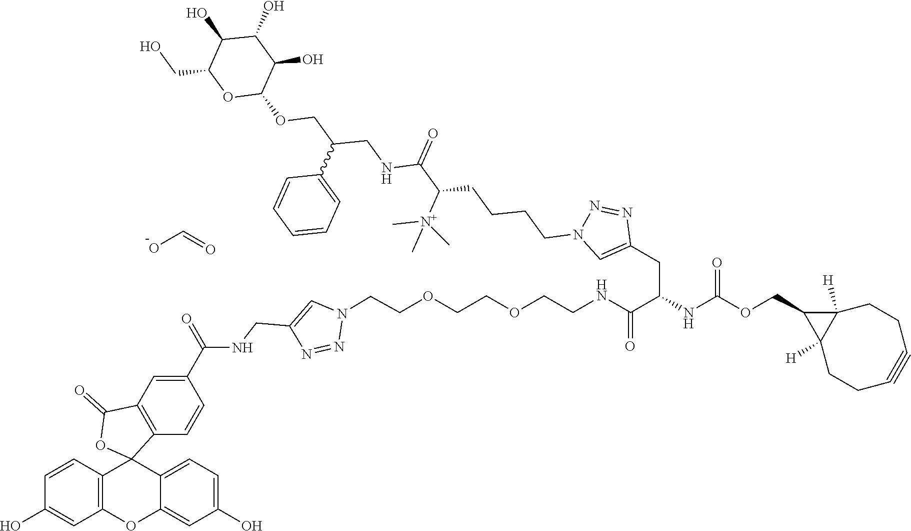 [SMILES: C[N+](C)(C)[C@@H](CCCCn1cc(C[C@H](NC(=O)OC[C@@H]2[C@@H]3CCC#CCC[C@@H]32)C(=O)NCCOCCOCCn2cc(CNC(=O)c3ccc4c(c3)C(=O)OC43c4ccc(O)cc4Oc4cc(O)ccc43)nn2)nn1)C(=O)NCC(CO[C@@H]1O[C@H](CO)[C@@H](O)[C@H](O)[C@H]1O)c1ccccc1 ; O=CO]

Prepared according to General Procedure C from I99 (3.5 mg, 2.73 μmol), (1R,8S,9s)-Bicyclo[6.1.0]non-4-yn-9-ylmethyl N-succinimidyl carbonate (2.5 mg, 8.58 μmol), NaHCO₃ (3 mg, 36 μmol) in MeCN (0.25 mL) and water (0.25 mL). Purified by RP MPLC (H₂O with increasing MeCN (0.1% formic acid)) to give the product as an orange solid (2.2 mg, 1.55 μmol, 57%).

MS (electrospray, +ve), [M]⁺ calculated for C₇₀H₈₆N₁₁O₁₈ requires: 1368.6147, found: 1368.5.

### Example 92: Synthesis of FIM48

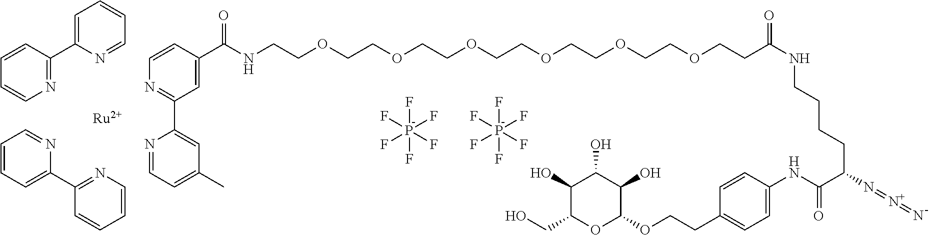

Bis(2,2′-bipyridine)-4′-methyl-4-carboxybipyridine-ruthenium N-succinimidyl ester-bis(hexafluorophosphate) (1.5 mg, 1.5 μmol) dissolved in DMF (0.3 mL) with DIPEA (1 μL, 5.7 μmol) and I72 (1.6 mg, 1.8 μmol) added. Reaction stirred for 2 hours then solvents removed under reduced pressure. Residue purified by RP MPLC (H₂O with increasing MeCN (0.1% formic acid)) to give the product as a red solid (2.1 mg, 0.80 μmol, 54%).

MS (electrospray, +ve), [M]²⁺ calculated for C₆₇H₈₄N₁₂O₁₅Ru requires: 699.2606, found: 699.3.

### Example 93: Synthesis of FIM49

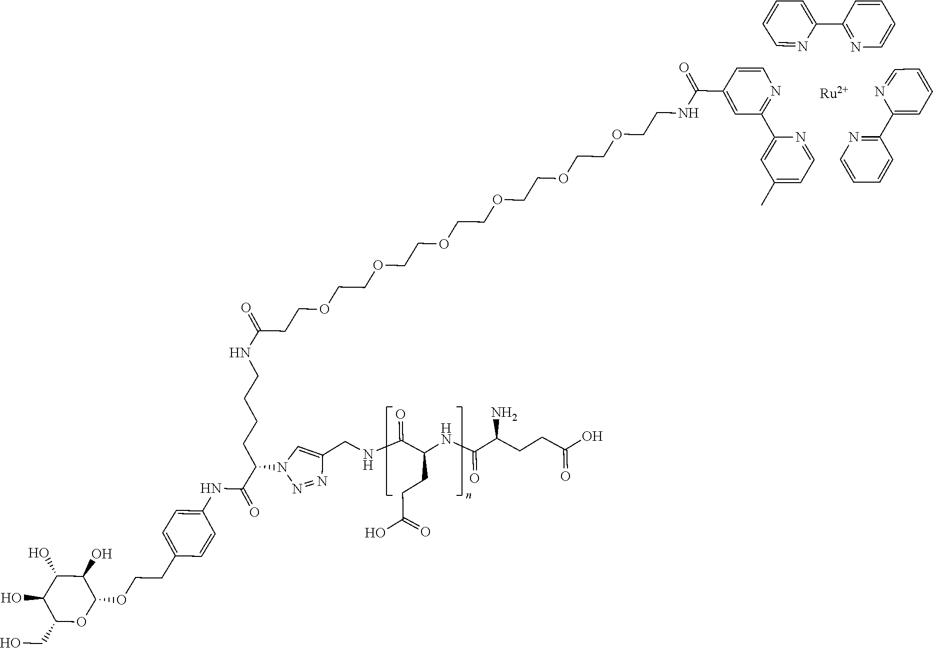

n=−154

Prepared according to General Procedure B from FIM48 (0.4 mg, 0.225 μmol), Alkyne-Poly(L-Glu) (154) (4 mg, 0.200 μmol), sodium ascorbate (1 mg, 4.0 μmol), CuSO₄·5H₂O (0.2 mg, 1.0 μmol) and NaHCO₃ (3.2 mg, 38.0 μmol) in water (0.5 mL). Product obtained by dialysis (6k MWCO) of reaction mixture in water (1.8 L), followed by lyophilisation to give an orange powder (4.0 mg, 0.184 μmol, 90%).

Absₘₐₓ=450 nm. Emₘₐₓ=660 nm.

### Example 94: Synthesis of FIM50

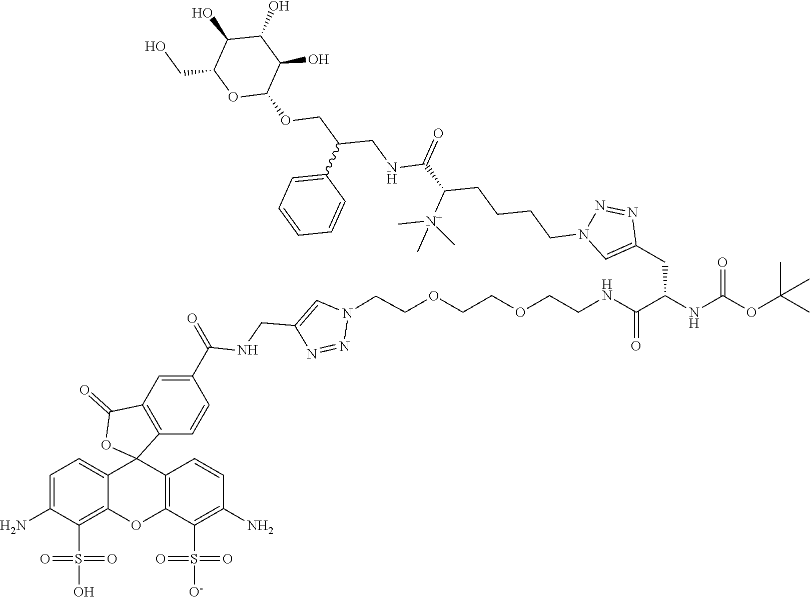 [SMILES: CC(C)(C)OC(=O)N[C@@H](Cc1cn(CCCC[C@@H](C(=O)NCC(CO[C@@H]2O[C@H](CO)[C@@H](O)[C@H](O)[C@H]2O)c2ccccc2)[N+](C)(C)C)nn1)C(=O)NCCOCCOCCn1cc(CNC(=O)c2ccc3c(c2)C(=O)OC32c3ccc(N)c(S(=O)(=O)[O-])c3Oc3c2ccc(N)c3S(=O)(=O)O)nn1]

Prepared according to General Procedure B from AF488-alkyne (3 mg, 5.2 μmol), I89 (5 mg, 5.4 μmol), sodium ascorbate (5 mg, 26 μmol), CuSO₄·5H₂O (1 mg, 4.2 μmol) in water (0.5 mL). Purified by RP MPLC (H₂O with increasing MeCN (0.1% formic acid)) to give the product as an orange solid (4.8 mg, 3.2 μmol, 61%).

MS (electrospray, +ve), [M+H]⁺ calculated for C₆₄H₈₄N₁₃O₂₂S₂ requires: 1450.5290, found: 1450.5.

### Example 95: Synthesis of FIM51

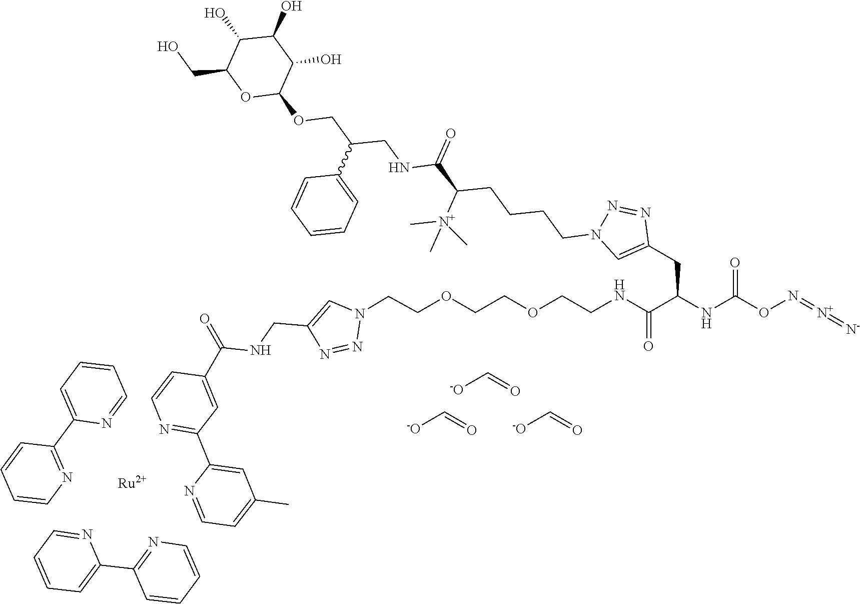

Prepared according to General Procedure C from I101 (5 mg, 3.1 μmol), azidoacetic acid NHS ester (3 mg, 15 μmol) and DIPEA (1 μL, 5.7 μmol) in DMF (0.6 mL). Purified by RP HPLC (H₂O with increasing MeCN (0.1% formic acid)). Product obtained as a red solid (2.7 mg, 1.6 μmol, 52%).

MS (electrospray, +ve), [M]³⁺ calculated for C₇₂H₈₉N₂₀O₁₂Ru requires: 509.1999, found: 509.2.

### Example 96: Synthesis of FIM52

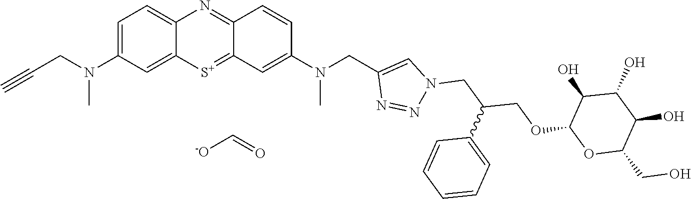

Prepared according to General Procedure B from I102 (9 mg, 19.6 μmol), I96 (8 mg, 15.8 μmol), sodium ascorbate (3 mg, 13.6 μmol), copper iodide (3 mg, 12 μmol) and tris(3-hydroxypropyltriazolylmethyl)amine (7 mg, 15.8 μmol) in THF (0.5 mL) and water (0.2 mL). The reaction mixture was passed through a silica plug eluting with MeOH containing 3% NH₃. The resultant blue solution was concentrated under reduced pressure and the residue dissolved in MeOH (2 mL). Sodium methoxide (1 mg, 20.7 μmol) was added and stirred for 2 minutes whereupon the reaction was quenched with formic acid. This reaction mixture was purified by RP MPLC (H₂O with increasing MeCN (0.1% formic acid)) to give the product as a blue solid (4 mg, 4.1 μmol, 26%).

MS (electrospray, +ve), [M-(C₃H₂)]⁺ calculated for C₃₂H₃₇N₆O₆S requires: 633.2490, found: 633.2.

### Example 97: Synthesis of FIM53

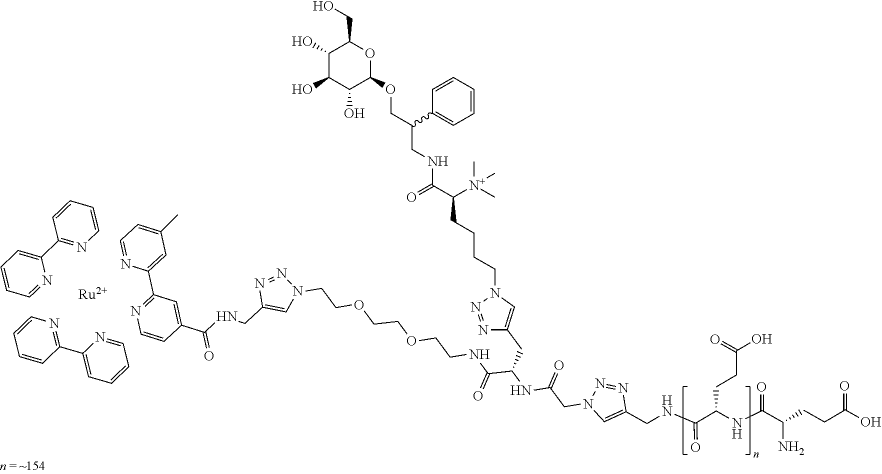

Prepared according to General Procedure B from FIM51 (1.4 mg, 0.825 μmol), Alkyne-Poly(L-Glu) (154) (15 mg, 0.750 μmol), sodium ascorbate (3 mg, 15.0 μmol), CuSO₄·5H₂O (0.9 mg, 3.8 μmol) and NaHCO₃ (12 mg, 139 μmol) in water (0.75 mL) and THF (0.75 mL). Product obtained by dialysis (6k MWCO) of reaction mixture in water (1.8 L), followed by lyophilisation to give an orange powder (9.3 mg, 0.430 μmol, 57%).

Absₘₐₓ=450 nm. Emₘₐₓ=660 nm.

### Example 98: Synthesis of FIM54

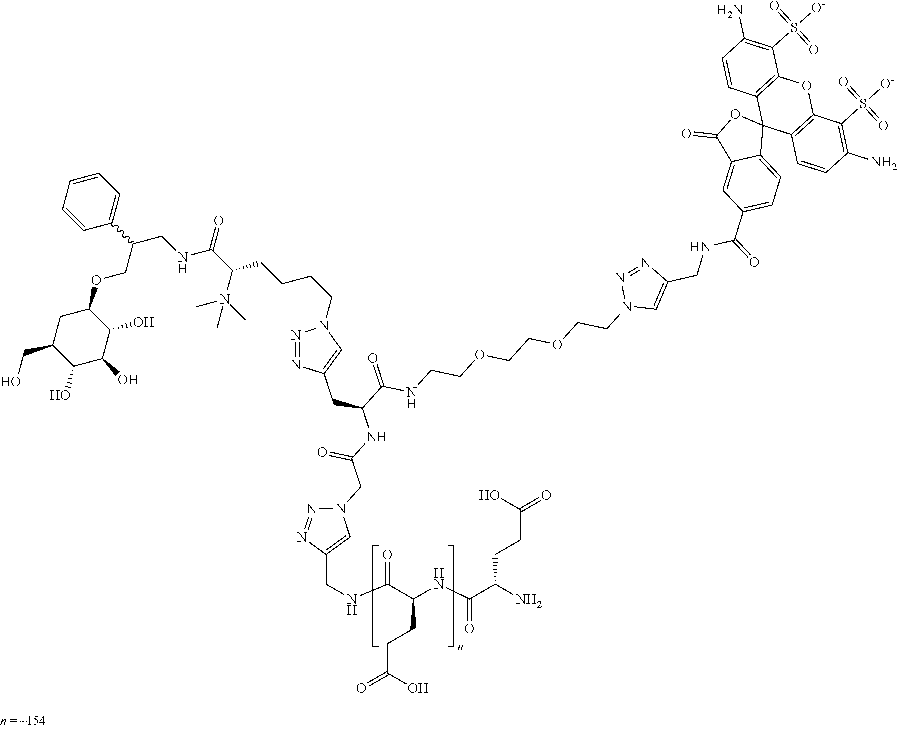

Prepared according to General Procedure B from I103 (2.5 mg, 1.7 μmol), Alkyne-Poly(L-Glu) (154) (30 mg, 1.5 μmol), sodium ascorbate (6 mg, 30.0 μmol), CuSO₄·5H₂O (2 mg, 7.5 μmol) and NaHCO₃ (23 mg, 277 μmol) in water (1.5 mL) and THF (1.5 mL). Product obtained by dialysis (6k MWCO) of reaction mixture in water (1.8 L), followed by lyophilisation to give an orange powder (19 mg, 0.89 μmol, 59%).

Absₘₐₓ=480 nm. Emₘₐₓ=520 nm.

### Example 99: Synthesis of FIM55

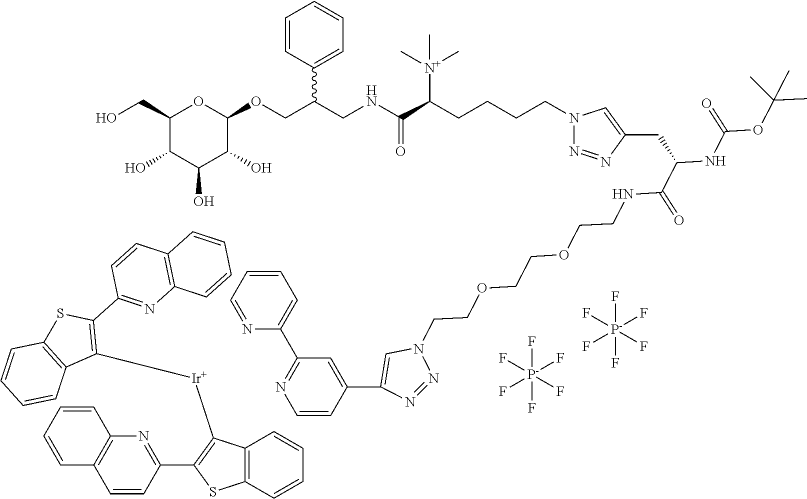

Prepared according to General Procedure B from I89 (4.0 mg, 4.3 μmol), I105 (3.0 mg, 2.9 μmol), CuSO₄·5H₂O (0.5 mg, 2.0 μmol), sodium ascorbate (3.0 mg, 15.1 μmol) and NaHCO₃ (3.0 mg, 35.7 μmol) in water (0.5 mL) and THF (0.5 mL). Purified by addition of ammonium hexafluorophosphate followed by RP MPLC (H₂O with increasing MeCN (0.1% formic acid)). Product obtained as an orange solid (4.0 mg, 2.0 μmol, 71%).

MS (electrospray, +ve), [M]²⁺ calculated for C₈₆H₉₅IrN₁₄O₁₂S₂ requires: 886.3157, found: 886.3.

### Example 100: Synthesis of FIM56

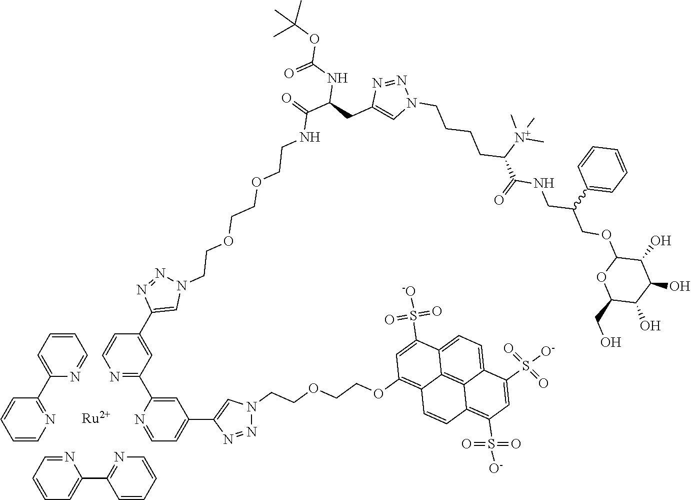

Prepared according to General Procedure B from I89 (1.6 mg, 1.8 μmol), I111 (1.5 mg, 1.2 μmol), CuSO₄·5H₂O (0.2 mg, 0.83 μmol) and sodium ascorbate (1.2 mg, 6.2 μmol) in water (0.25 mL) and THF (0.25 mL). Solvents removed and the residue purified by RP MPLC (H₂O with increasing MeCN (0.1% formic acid)). Product obtained as a yellow solid (1.8 mg, 0.87 μmol, 73%).

MS (electrospray, +ve), [M+H]⁺ calculated for C₉₄H₁₀₆N₁₉O₂₃RuS₃ requires: 2067.5943, found: 2067.5.

### Example 101: Synthesis of FIM57

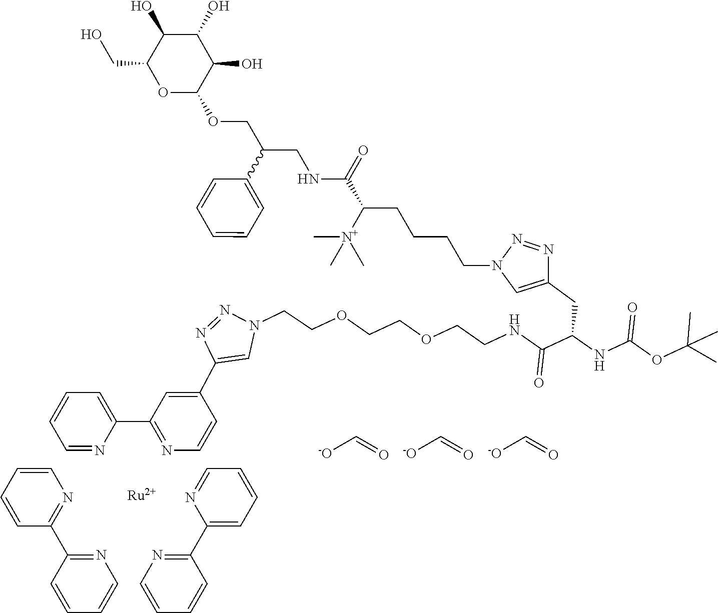

Prepared according to General Procedure B from I89 (4.7 mg, 5.1 μmol), I110 (3.4 mg, 5.1 μmol), CuSO₄·5H₂O (0.5 mg, 2.0 μmol) and sodium ascorbate (3.0 mg, 15.2 μmol) in water (0.7 mL). Solvents removed and the residue purified by RP MPLC (H₂O with increasing MeCN (0.1% formic acid)). Product obtained as an orange solid (3.2 mg, 2.0 μmol, 39%).

MS (electrospray, +ve), [M]³⁺ calculated for C₇₂H₉₁N₁₆O₁₂Ru requires: 491.2010, found: 491.2.

### Example 102: Synthesis of FIM58

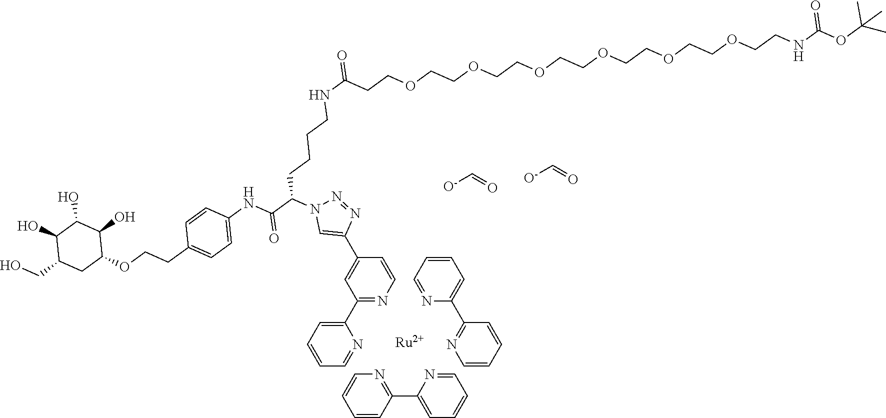

Prepared according to General Procedure B from I30 (10 mg, 11.2 μmol), I110 (7.5 mg, 11.2 μmol), CuSO₄·5H₂O (1.4 mg, 5.6 μmol) and sodium ascorbate (11 mg, 56.2 μmol) in water (0.8 mL). Solvents removed and the residue purified by RP MPLC (H₂O with increasing MeCN (0.1% formic acid)). Product obtained as an orange solid (12 mg, 7.7 μmol, 68%).

MS (electrospray, +ve), [M]²⁺ calculated for C₇₂H₉₂N₁₂O₁₆Ru requires: 741.2894, found: 741.3.

### Example 103: Synthesis of FIM59

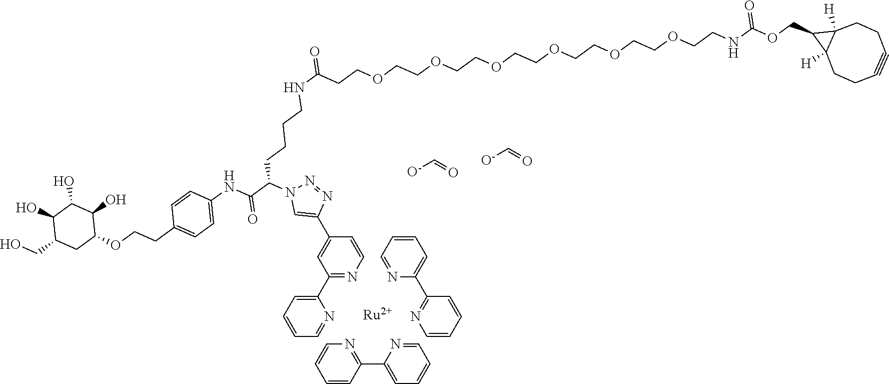

Prepared according to General Procedure C from I114 (1.5 mg, 1.0 μmol), (1R,8S,9s)-bicyclo[6.1.0]non-4-yn-9-ylmethyl N-succinimidyl carbonate (1.5 mg, 5.0 μmol) and NaHCO₃ (1 mg, 10.0 μmol) in MeCN (0.4 mL) and water (0.4 mL). Purified by RP MPLC (H₂O with increasing MeCN (0.1% formic acid)). Product obtained as an orange solid (1.5 mg, 0.9 μmol, 90%).

MS (electrospray, +ve), [M]²⁺ calculated for C₇₃H₉₆N₁₂O₁₆Ru requires: 779.3050, found: 779.3.

### Example 104: Synthesis of FIM60

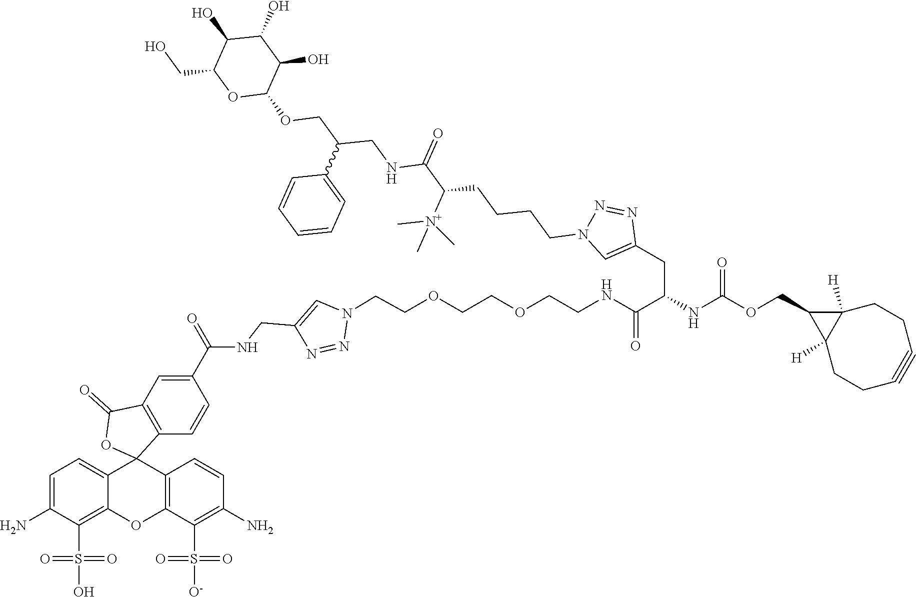 [SMILES: C[N+](C)(C)[C@@H](CCCCn1cc(C[C@H](NC(=O)OC[C@@H]2[C@@H]3CCC#CCC[C@@H]32)C(=O)NCCOCCOCCn2cc(CNC(=O)c3ccc4c(c3)C(=O)OC43c4ccc(N)c(S(=O)(=O)[O-])c4Oc4c3ccc(N)c4S(=O)(=O)O)nn2)nn1)C(=O)NCC(CO[C@@H]1O[C@H](CO)[C@@H](O)[C@H](O)[C@H]1O)c1ccccc1]

Prepared according to General Procedure C from I104 (1.4 mg, 0.89 μmol), (1R,8S,9s)- 15 bicyclo[6.1.0]non-4-yn-9-ylmethyl N-succinimidyl carbonate (1.3 mg, 4.4 μmol) and NaHCO₃ (1 mg, 10.0 μmol) in MeCN (0.4 mL) and water (0.4 mL). Purified by RP MPLC (H₂O with increasing MeCN (0.1% formic acid)). Product obtained as a yellow solid (1.5 mg, 0.9 μmol, 90%).

MS (electrospray, +ve), [M+H]⁺ calculated for C₇₀H₈₈N₁₃O₂₂S₂ requires: 1526.5603, found: 1526.4.

### Example 105: Synthesis of FIM61

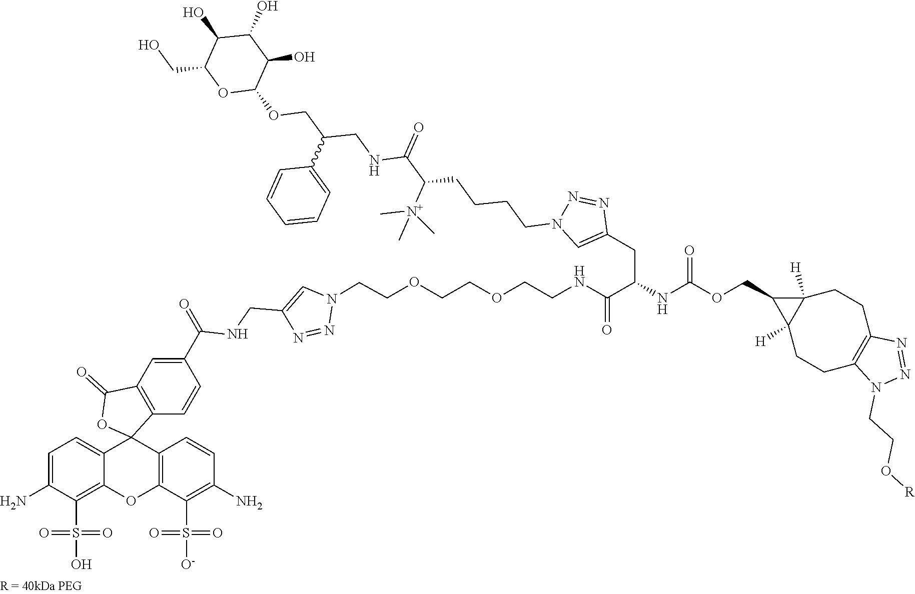

FIM60 (3 mg, 1.8 μmol) and 40 kDa mPEG-N₃ (104 mg, 2.6 μmol) were dissolved in water (1.8 mL) and stirred for 24 h. The reaction mixture was transferred to a Vivaspin centrifugal concentrator (10 kDa MWCO, PES). Sample was washed exhaustively with water until filtrate was colourless before freeze drying to give the product, as a mixture with the excess 40 kDa mPEG-N₃, as an orange solid (101 mg, 2.5 μmol, 96%).

Absₘₐₓ=488 nm. Emₘₐₓ=520 nm.

### Example 106: Synthesis of FIM62

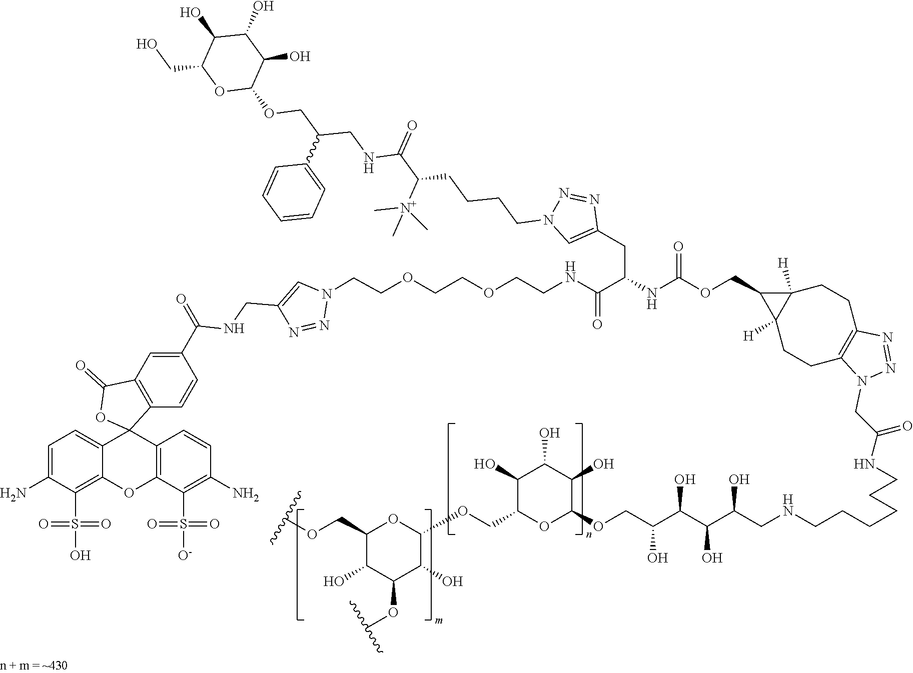

To a solution of I115 (8 mg, 0.114 μmol) in water (0.7 mL), was added an aq. solution of FIM60 (1.0 mM, 126 μL, 0.126 μmol) and the reaction stirred for 4 h. The reaction mixture was dialysed (6000 MWCO) from 20% aq. EtOH and the resultant solution freeze dried to give the product as an orange solid (7 mg, 0.98 μmol, 86%).

Absₘₐₓ=488 nm. Emₘₐₓ=520 nm.

### Example 107: Synthesis of FIM63

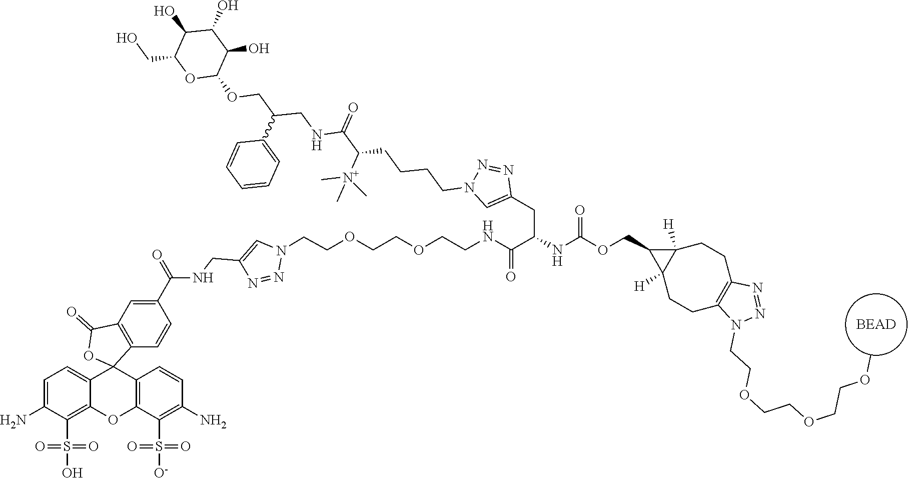

To azide agarose beads (52 μL aq. suspension, −19 mM wrt azide) was added a solution of FIM60 (1.0 mM, 50 μL, 0.05 μmol) and the suspension was frequently vortexed. After 30 min the suspension was diluted to 1 mL and centrifuged. Supernatant was removed, and pellet resuspended in 1 mL and centrifuged again. Supernatant removed again, and the remaining orange beads were resuspended in 10 mM PBS (1 mL).

Absₘₐₓ=488 nm. Emₘₐₓ=520 nm

### Example 108: Synthesis of FIM64

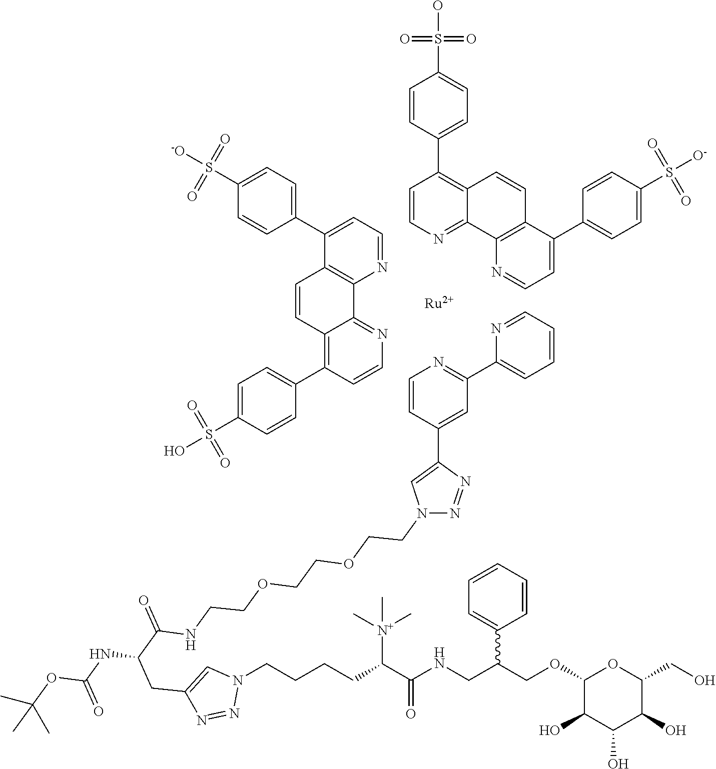

Prepared according to General Procedure B from I89 (3.2 mg, 3.4 μmol), I116 (12 mg, 9.0 μmol), CuSO₄·5H₂O (0.6 mg, 2.6 μmol), sodium ascorbate (6.4 mg, 33 μmol) and NaHCO₃ (1 mg, 10.0 μmol) in water (0.5 mL) and MeCN (0.01 mL). Solvents removed and the residue purified by RP MPLC (H₂O with increasing MeCN (0.1% formic acid)) followed by RP HPLC to give the product as a yellow solid (2 mg, 0.90 μmol, 28%).

MS (electrospray, +ve), [M+2H]²⁺ calculated for C₁₀₀H₁₀₆N16O₂₄RuS₄ requires: 1072.7758, found: 1072.8.

### Example 109: Synthesis of FIM65

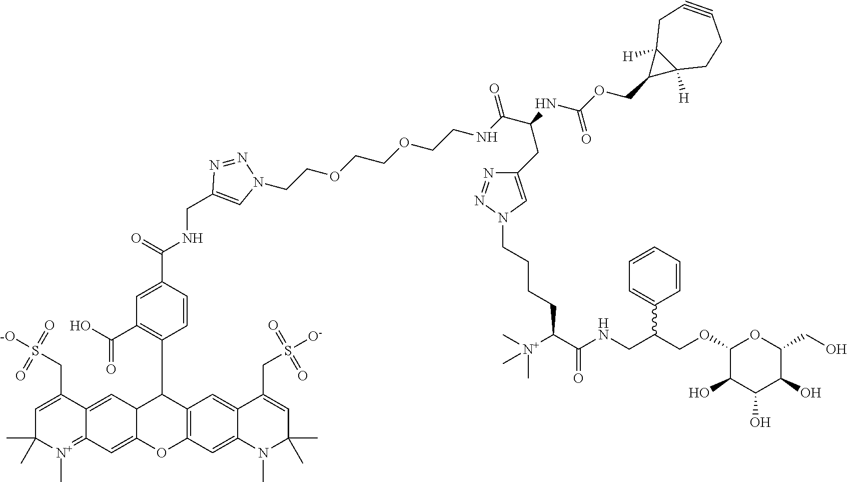

I118 (3.3 mg, 2.0 μmol) and (1R,8S,9s)- 15 bicyclo[6.1.0]non-4-yn-9-ylmethyl N-succinimidyl carbonate (1.7 mg, 6.0 μmol) dissolved in DMF (1 mL) with DIPEA (3 μL, 17.0 μmol) and reaction stirred for 20 h. Solvents removed under reduced pressure, and the residue purified by RP MPLC (H₂O with increasing MeCN (0.1% formic acid)). Product obtained as a blue solid (2.5 mg, 1.5 μmol, 73%).

MS (electrospray, +ve), [M+2H]²⁺ calculated for C₃₄H₁₁₁N₁₃O₂₂S₂ requires: 858.8699, found: 858.4.

### Example 110: Synthesis of FIM66

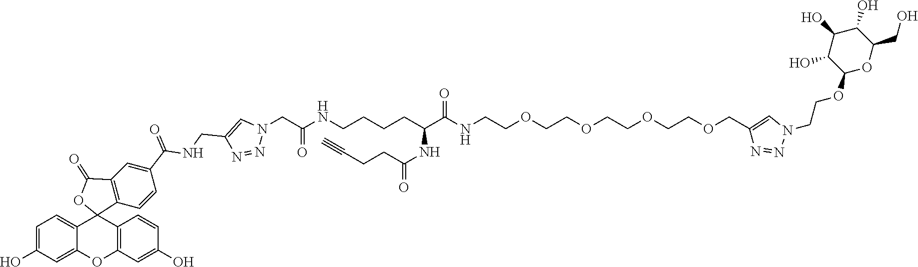 [SMILES: C#CCCC(=O)N[C@@H](CCCCNC(=O)Cn1cc(CNC(=O)c2ccc3c(c2)C(=O)OC32c3ccc(O)cc3Oc3cc(O)ccc32)nn1)C(=O)NCCOCCOCCOCCOCc1cn(CCO[C@@H]2O[C@H](CO)[C@@H](O)[C@H](O)[C@H]2O)nn1]

Prepared according to General Procedure C from I119 (10 mg, 8.2 μmol), pentynoic acid N-hydroxysuccinimide ester (8 mg, 41 μmol) and NaHCO₃ (3 mg, 33 μmol) in MeCN (1.3 mL) and water (2.1 mL). Purified by RP MPLC (H₂O with increasing MeCN (0.1% formic acid)). Product obtained as an orange solid (7 mg, 5.9 μmol, 92%). MS (electrospray, +ve), [M+H]⁺ calculated for C₅₆H₆₉N₁₀O₁₉ requires: 1185.4735, found: 1185.4.

### Example 111: Synthesis of FIM67

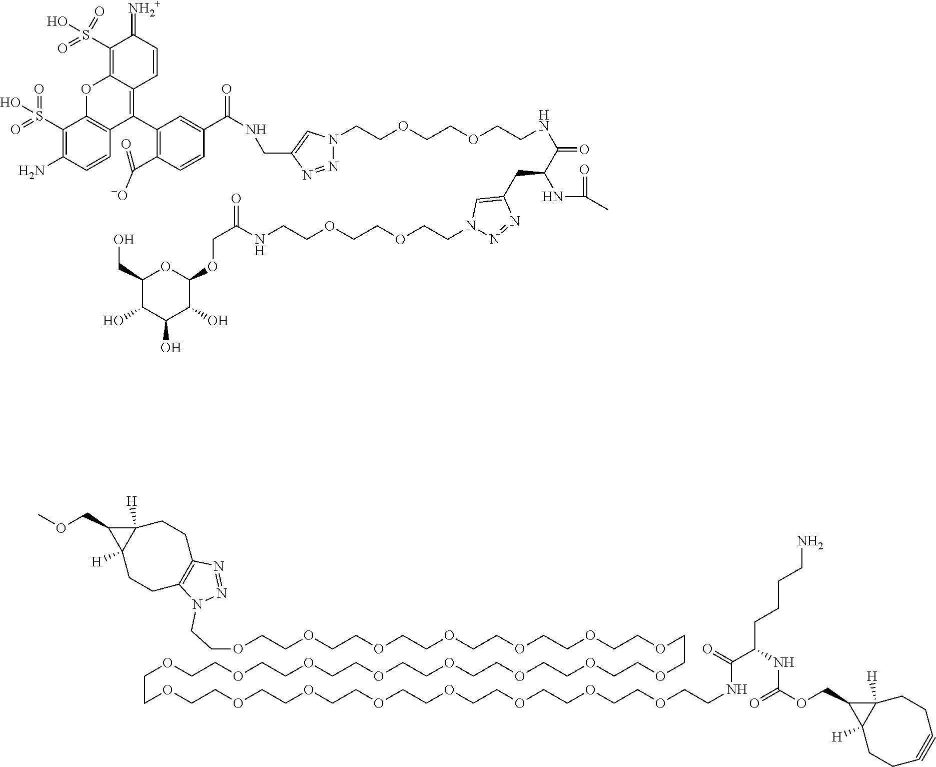

To a solution of I125 (1 mg, 0.329 μmol) in DMF (0.5 mL) was added piperidine (5 μL, 50.6 μmol). Reaction stirred for 20 min at RT before solvents were removed under a stream of N₂. Purified by RP MPLC (H₂O with increasing MeCN (0.1% formic acid)). Product obtained as an orange solid (0.8 mg, 0.280 μmol, 85%).

MS (electrospray, +ve) [M+3Na]³⁺ calculated for C₁₂₅H₁₉₆N₁₃Na₃O₅₀S₂ requires: 961.0833, found: 961.2.

### Example 112: Synthesis of FIM68

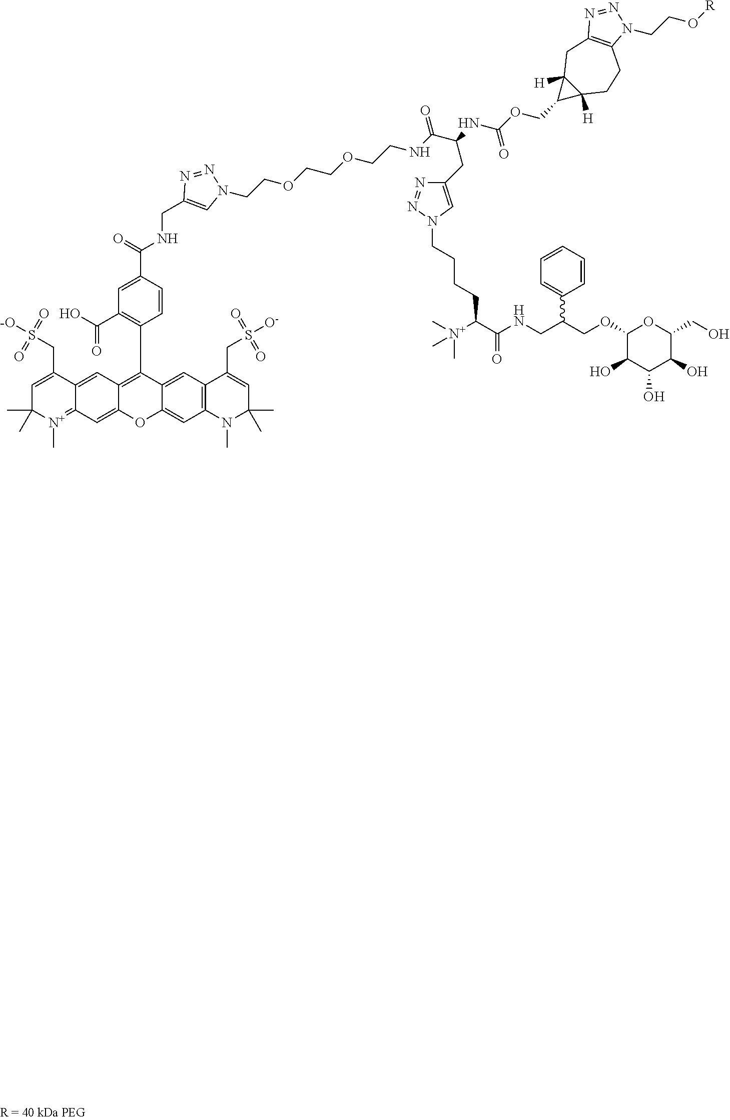

FIM65 (0.5 mg, 0.286 μmol) and 40 kDa mPEG-N₃ (14 mg, 0.348 μmol) were dissolved in water (1.8 mL) and stirred for 48 h. The reaction mixture was transferred to a Vivaspin centrifugal concentrator (10 kDa MWCO, PES). Sample was washed exhaustively with water until filtrate was colourless before freeze drying to give the product, as a mixture with the excess 40 kDa mPEG-N₃, as a purple solid (13 mg, 7.1 μmol, 92%).

Absₘₐₓ=594 nm. Emₘₐₓ=620 nm.

### Fluorescent Inhibitor Molecules (FIM) Intermediates (IX) Synthetic Procedures

### Synthesis of I1

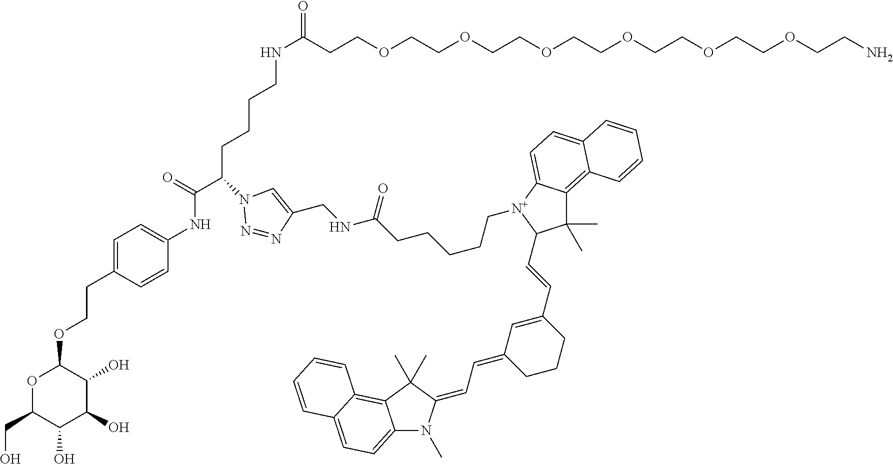 [SMILES: CN1/C(=C/C=C2C=C(/C=C/C3[NH+](CCCCCC(=O)NCc4cn([C@@H](CCCCNC(=O)CCOCCOCCOCCOCCOCCOCCN)C(=O)Nc5ccc(CCO[C@@H]6O[C@H](CO)[C@@H](O)[C@H](O)[C@H]6O)cc5)nn4)c4ccc5ccccc5c4C3(C)C)CCC/2)C(C)(C)c2c1ccc1ccccc21]

Prepared according to General Procedure B from I72 (7.0 mg, 0.009 mmol), Cy7.5 alkyne (5.0 mg, 0.007 mmol), CuSO₄·5H₂O (0.3 mg, 0.001 mmol), sodium ascorbate (4.0 mg, 0.021 mmol) in THF (1 mL) and water (1 mL). Purified by RP MPLC (H₂O with increasing MeCN (0.1% formic acid)). Product obtained as a green solid (6.0 mg, 0.004 mmol, 59%)

MS (electrospray, +ve), [M+H]⁺ calculated for C₈₃H₁₁₂N₉O₁₅ requires: 1474.828, found: 1474.7

### Synthesis of I2

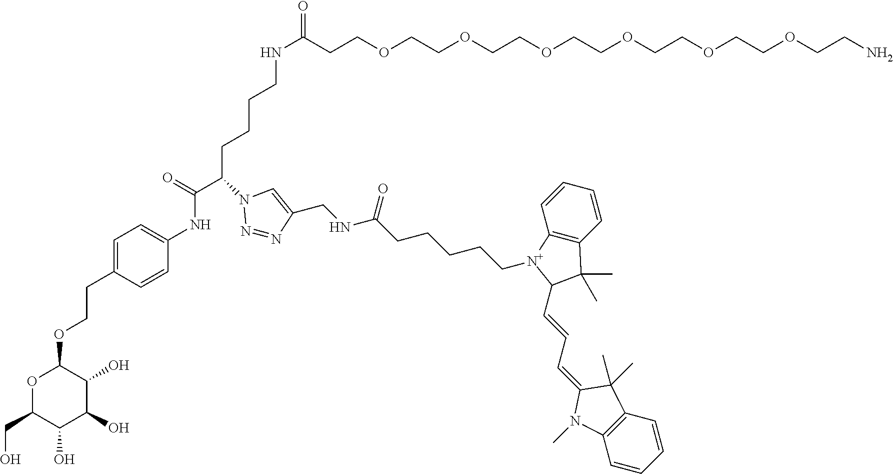 [SMILES: CN1/C(=C/C=C/C2[NH+](CCCCCC(=O)NCc3cn([C@@H](CCCCNC(=O)CCOCCOCCOCCOCCOCCOCCN)C(=O)Nc4ccc(CCO[C@@H]5O[C@H](CO)[C@@H](O)[C@H](O)[C@H]5O)cc4)nn3)c3ccccc3C2(C)C)C(C)(C)c2ccccc21]

Prepared according to General Procedure B from I72 (7.0 mg, 0.009 mmol), Cy3 alkyne (5.0 mg, 0.007 mmol), CuSO₄·5H₂O (0.3 mg, 0.001 mmol), sodium ascorbate (4.0 mg, 0.021 mmol) in THF (1 mL) and water (1 mL). Purified by RP MPLC (H₂O with increasing MeCN (0.1% formic acid)). Product obtained as a green solid (6.0 mg, 0.004 mmol, 59%)

MS (electrospray, +ve), [M+H]⁺ calculated for C₈₃H₁₁₂N₉O₁₅ requires: 1282.734, found: 1282.6

### Synthesis of I3

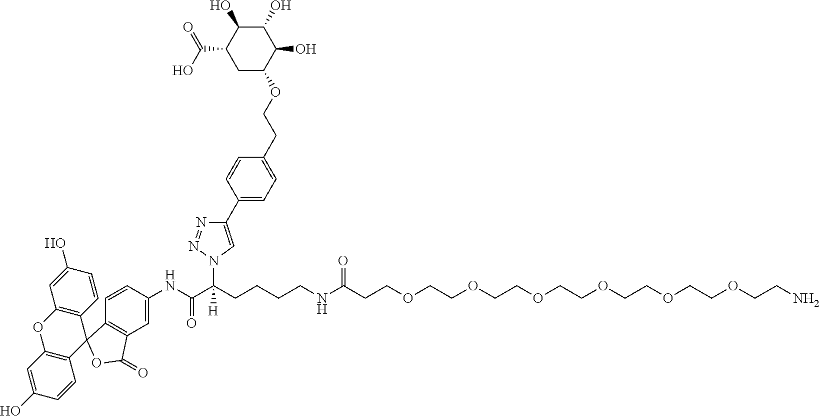 [SMILES: NCCOCCOCCOCCOCCOCCOCCC(=O)NCCCC[C@@H](C(=O)Nc1ccc2c(c1)C(=O)OC21c2ccc(O)cc2Oc2cc(O)ccc21)n1cc(-c2ccc(CCO[C@@H]3C[C@H](C(=O)O)[C@@H](O)[C@H](O)[C@H]3O)cc2)nn1]

Prepared according to General Procedure D from I4 (11 mg, 0.009 mmol) in TFA (0.1 mL) and DCM (0.1 mL). Product obtained as an orange solid (10 mg, 0.009 mmol, 98%).

MS (electrospray, +ve), [M+H]⁺ calculated for C₅₇H₇₁N₆O₂₀ requires: 1159.472, found: 1159.4

### Synthesis of I4

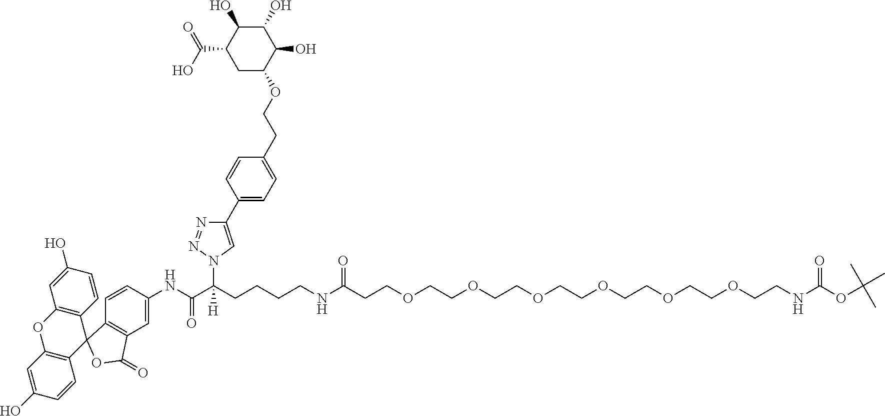 [SMILES: CC(C)(C)OC(=O)NCCOCCOCCOCCOCCOCCOCCC(=O)NCCCC[C@@H](C(=O)Nc1ccc2c(c1)C(=O)OC21c2ccc(O)cc2Oc2cc(O)ccc21)n1cc(-c2ccc(CCO[C@@H]3C[C@H](C(=O)O)[C@@H](O)[C@H](O)[C@H]3O)cc2)nn1]

Prepared according to General Procedure A from I5 (7.3 mg, 0.016 mmol), 21-(Boc-amino)-4,7,10,13,16,19-hexaoxaheneicosanoic acid (11 mg, 0.013 mmol), HBTU (6 mg, 0.016 mmol), HOBt·H₂O (4 mg, 0.027 mmol) and DIPEA (9 μL, 0.053 mmol) in DMF (1 mL). Purified by RP MPLC (H₂O with increasing MeCN (0.1% formic acid)) to give the product as an orange solid (13 mg, 0.010 mmol, 78%).

MS (electrospray, +ve), [M+H]⁺ calculated for C₆₂H₇₉N₆O₂₂ requires: 1259.5242, found: 1259.4

### Synthesis of I5

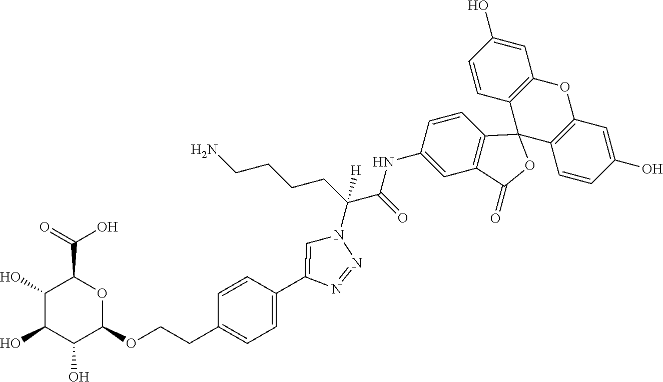 [SMILES: NCCCC[C@@H](C(=O)Nc1ccc2c(c1)C(=O)OC21c2ccc(O)cc2Oc2cc(O)ccc21)n1cc(-c2ccc(CCO[C@@H]3O[C@H](C(=O)O)[C@@H](O)[C@H](O)[C@H]3O)cc2)nn1]

Prepared according to General Procedure D from I6 (15 mg, 0.016 mmol) in TFA (0.2 mL) and DCM (0.2 mL). Product obtained as an orange solid (12 mg, 0.015 mmol, 90%).

MS (electrospray, +ve), [M+H]⁺ calculated for C₄₂H₄₁N₅O₁₃ requires: 824.2779, found: 824.4

### Synthesis of I6

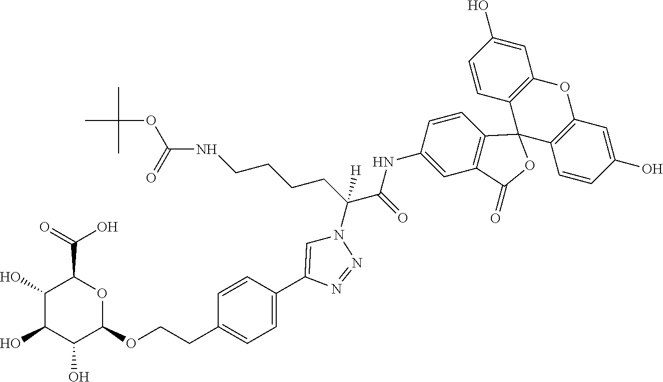 [SMILES: CC(C)(C)OC(=O)NCCCC[C@@H](C(=O)Nc1ccc2c(c1)C(=O)OC21c2ccc(O)cc2Oc2cc(O)ccc21)n1cc(-c2ccc(CCO[C@@H]3O[C@H](C(=O)O)[C@@H](O)[C@H](O)[C@H]3O)cc2)nn1]

Prepared according to General Procedure B from I54 (20 mg, 0.033 mmol), I46 (13 mg, 0.040 mmol), CuSO₄·5H₂O (3.3 mg, 0.013 mmol), sodium ascorbate (20 mg, 0.10 mmol) in THF (3 mL) and water (3 mL). Purified by RP MPLC (H₂O with increasing MeCN (0.1% formic acid)). Product obtained as an orange solid (15 mg, 0.016 mmol, 49%)

MS (electrospray, +ve), [M+H]⁺ calculated for C₄₇H₅₀N₅O₁₅ requires: 924.3303, found: 924.3

### Synthesis of I7

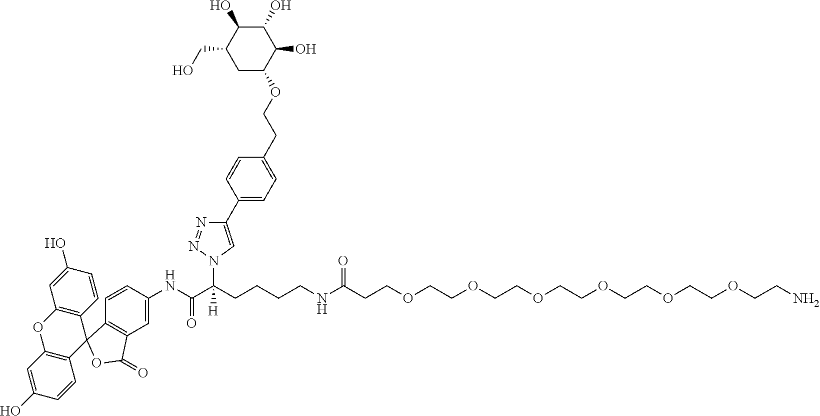 [SMILES: NCCOCCOCCOCCOCCOCCOCCC(=O)NCCCC[C@@H](C(=O)Nc1ccc2c(c1)C(=O)OC21c2ccc(O)cc2Oc2cc(O)ccc21)n1cc(-c2ccc(CCO[C@@H]3C[C@H](CO)[C@@H](O)[C@H](O)[C@H]3O)cc2)nn1]

Prepared according to General Procedure D from I8 (10 mg, 0.009 mmol) in TFA (0.1 mL) and DCM (0.1 mL). Product obtained as an orange solid (10 mg, 0.009 mmol, 98%).

MS (electrospray, +ve), [M+H]⁺ calculated for C₅₇H₇₃N₆O₁₉ requires: 1145.493, found: 1145.4

### Synthesis of I8

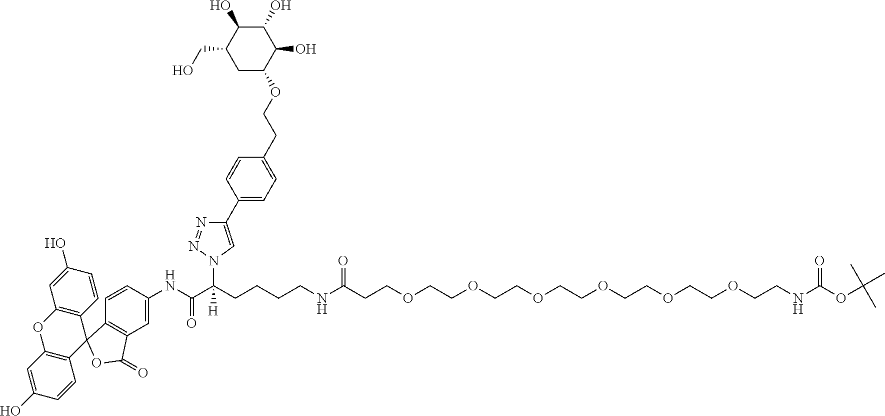 [SMILES: CC(C)(C)OC(=O)NCCOCCOCCOCCOCCOCCOCCC(=O)NCCCC[C@@H](C(=O)Nc1ccc2c(c1)C(=O)OC21c2ccc(O)cc2Oc2cc(O)ccc21)n1cc(-c2ccc(CCO[C@@H]3C[C@H](CO)[C@@H](O)[C@H](O)[C@H]3O)cc2)nn1]

Prepared according to General Procedure A from I9 (8.7 mg, 0.019 mmol), 21-(Boc-amino)-4,7,10,13,16,19-hexaoxaheneicosanoic acid (12 mg, 0.015 mmol), HBTU (5.6 mg, 0.015 mmol), HOBt·H₂O (2.3 mg, 0.015 mmol) and DIPEA (10 μL, 0.059 mmol) in DMF (1 mL). Purified by RP MPLC (H₂O with increasing MeCN (0.1% formic acid)) to give the product as an orange solid (10 mg, 0.008 mmol, 54%).

MS (electrospray, +ve), [M+H]⁺ calculated for C₆₂H₈₁N₆O₂₁ requires: 1245.5449, found: 1245.4

### Synthesis of I9

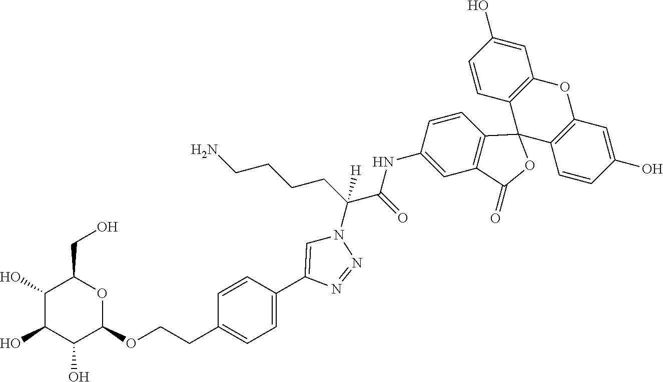 [SMILES: NCCCC[C@@H](C(=O)Nc1ccc2c(c1)C(=O)OC21c2ccc(O)cc2Oc2cc(O)ccc21)n1cc(-c2ccc(CCO[C@@H]3O[C@H](CO)[C@@H](O)[C@H](O)[C@H]3O)cc2)nn1]

Prepared according to General Procedure D from I10 (13 mg, 0.014 mmol) in TFA (0.2 mL) and DCM (0.2 mL). Product obtained as an orange solid (11 mg, 0.014 mmol, 95%).

MS (electrospray, +ve), [M+H]⁺ calculated for C₄₂H₄₄N₅O₁₂ requires: 810.2986, found: 810.2

### Synthesis of I10

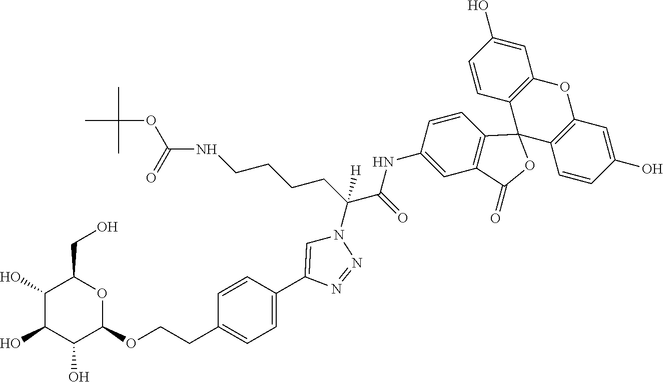 [SMILES: CC(C)(C)OC(=O)NCCCC[C@@H](C(=O)Nc1ccc2c(c1)C(=O)OC21c2ccc(O)cc2Oc2cc(O)ccc21)n1cc(-c2ccc(CCO[C@@H]3O[C@H](CO)[C@@H](O)[C@H](O)[C@H]3O)cc2)nn1]

Prepared according to General Procedure B from I54 (20 mg, 0.033 mmol), I43 (13 mg, 0.040 mmol), CuSO₄·5H₂O (3.3 mg, 0.013 mmol), sodium ascorbate (20 mg, 0.10 mmol) in THF (3 mL) and water (3 mL). Purified by RP MPLC (H₂O with increasing MeCN (0.1% formic acid)). Product obtained as an orange solid (15 mg, 0.016 mmol, 49%)

MS (electrospray, +ve), [M+H]⁺ calculated for C₄₇H₅₂N₅O₁₄requires: 910.3511, found: 910.2

### Synthesis of I11

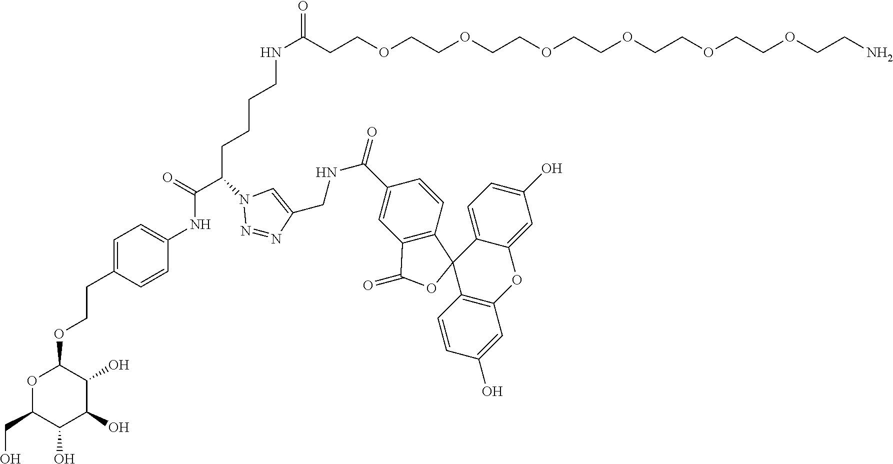 [SMILES: NCCOCCOCCOCCOCCOCCOCCC(=O)NCCCC[C@@H](C(=O)Nc1ccc(CCO[C@@H]2O[C@H](CO)[C@@H](O)[C@H](O)[C@H]2O)cc1)n1cc(CNC(=O)c2ccc3c(c2)C(=O)OC32c3ccc(O)cc3Oc3cc(O)ccc32)nn1]

Prepared according to General Procedure D from I12 (11 mg, 0.008 mmol) in TFA (0.1 mL) and DCM (0.1 mL). Product obtained as an orange solid (10 mg, 0.008 mmol, 98%).

MS (electrospray, +ve), [M+H]⁺ calculated for C₅₉H₇₆N₇O₂₀ requires: 1202.515, found: 1202.4

### Synthesis of I12

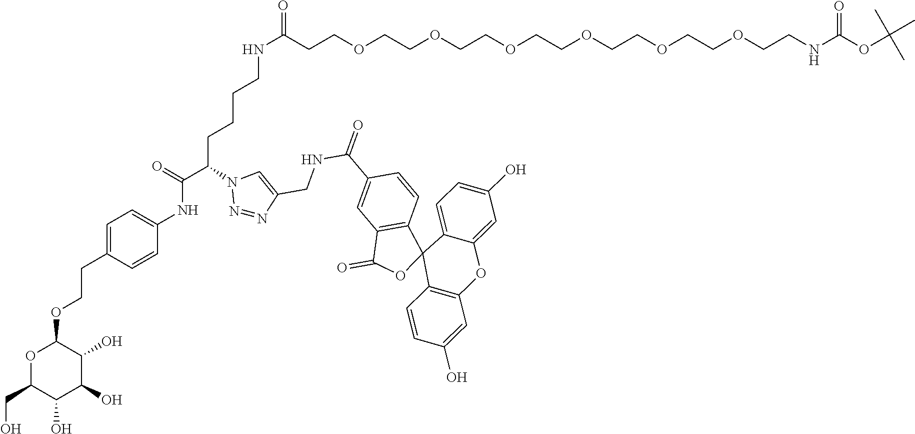 [SMILES: CC(C)(C)OC(=O)NCCOCCOCCOCCOCCOCCOCCC(=O)NCCCC[C@@H](C(=O)Nc1ccc(CCO[C@@H]2O[C@H](CO)[C@@H](O)[C@H](O)[C@H]2O)cc1)n1cc(CNC(=O)c2ccc3c(c2)C(=O)OC32c3ccc(O)cc3Oc3cc(O)ccc32)nn1]

Prepared according to General Procedure B from I30 (11 mg, 0.012 mmol), 5-FAM alkyne (5.5 mg, 0.013 mmol), CuSO₄·5H₂O (0.6 mg, 0.002 mmol), sodium ascorbate (7.2 mg, 0.036 mmol) in THF (1.1 mL) and water (1.1 mL). Purified by RP MPLC (H₂O with increasing MeCN (0.1% formic acid)). Product obtained as an orange solid (11 mg, 0.008 mmol, 70%).

MS (electrospray, +ve), [M+H]⁺ calculated for C₆₄H₈₄N₇O₂₂ requires: 1302.567, found: 1302.4

### Synthesis of I13

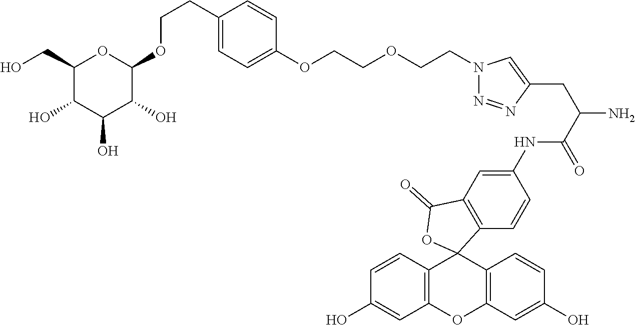 [SMILES: NC(Cc1cn(CCOCCOc2ccc(CCO[C@@H]3O[C@H](CO)[C@@H](O)[C@H](O)[C@H]3O)cc2)nn1)C(=O)Nc1ccc2c(c1)C(=O)OC21c2ccc(O)cc2Oc2cc(O)ccc21]

Prepared according to General Procedure D from I14 (25 mg, 0.026 mmol) in TFA (2 mL) and DCM (2 mL). Product obtained as an orange solid (22 mg, 0.026 mmol, 99%).

MS (electrospray, +ve), [M+H]⁺ calculated for C₄₃H₄₆N₅O₁₄ requires: 856.3041, found: 856.3

### Synthesis of I14

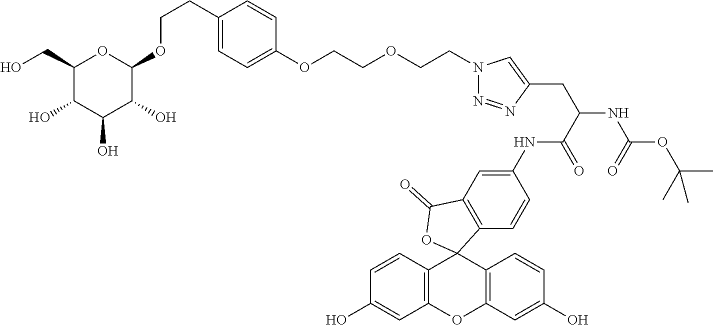 [SMILES: CC(C)(C)OC(=O)NC(Cc1cn(CCOCCOc2ccc(CCO[C@@H]3O[C@H](CO)[C@@H](O)[C@H](O)[C@H]3O)cc2)nn1)C(=O)Nc1ccc2c(c1)C(=O)OC21c2ccc(O)cc2Oc2cc(O)ccc21]

Prepared according to General Procedure B from I47 (100 mg, 0.242 mmol), I15 (157 mg, 0.290 mmol), CuSO₄·5H₂O (29 mg, 0.116 mmol), sodium ascorbate (91 mg, 0.460 mmol) in THF (4.8 mL) and water (2 mL). Purified by RP MPLC (H₂O with increasing MeCN (0.1% formic acid)). Product obtained as an orange solid (25 mg, 0.026 mmol, 11%)

¹H NMR (400 MHz, Methanol-d₄) δ 8.29 (d, J=2.0 Hz, 1H), 8.13 (s, 1H), 7.84 (s, 1H), 7.80 (d, J=8.3 Hz, 1H), 7.13 (d, J=8.4 Hz, 2H), 7.08 (d, J=8.2 Hz, 1H), 6.77 (d, J=8.6 Hz, 2H), 6.67 (d, J=2.3 Hz, 2H), 6.58 (dd, J=8.7, 2.5 Hz, 2H), 6.52 (dt, J=8.8, 2.2 Hz, 2H), 4.58-4.45 (m, 2H), 4.28 (d, J=7.8 Hz, 1H), 4.08-3.97 (m, 3H), 3.94-3.86 (m, 2H), 3.84 (s, 1H), 3.77-3.71 (m, 2H), 3.71-3.62 (m, 2H), 3.32 (d, J=1.8 Hz, 2H), 3.27 (d, J=6.9 Hz, 2H), 3.22-3.03 (m, 2H), 2.84 (t, J=7.0 Hz, 2H), 1.44 (s, 9H).

MS (electrospray, +ve), [M+H]⁺ calculated for C₄₈H₅₄N₅O₁₆ requires: 956.3566, found: 956.3.

### Synthesis of I15

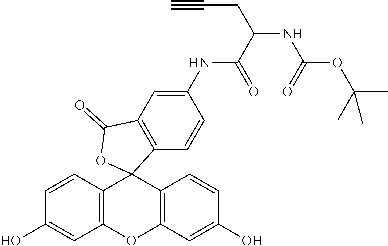 [SMILES: C#CCC(NC(=O)OC(C)(C)C)C(=O)Nc1ccc2c(c1)C(=O)OC21c2ccc(O)cc2Oc2cc(O)ccc21]

Prepared according to General Procedure A from Boc-L-2-propargylglycine (135 mg, 0.633 mmol), fluoresceinamine isomer I (200 mg, 0.0576 mmol), HBTU (262 mg, 0.691 mmol) and HOBt·H₂O (106 mg, 0.691 mmol) in pyridine (5) and THF (10 mL). Purified by RP MPLC (H₂O with increasing MeCN (0.1% formic acid)) to give the product as an orange solid (10 mg, 0.008 mmol, 54%).

MS (electrospray, +ve), [M+H]⁺ calculated for C₃₀H₂₇N₂O₈ requires: 543.1767, found: 543.2

### Synthesis of I16

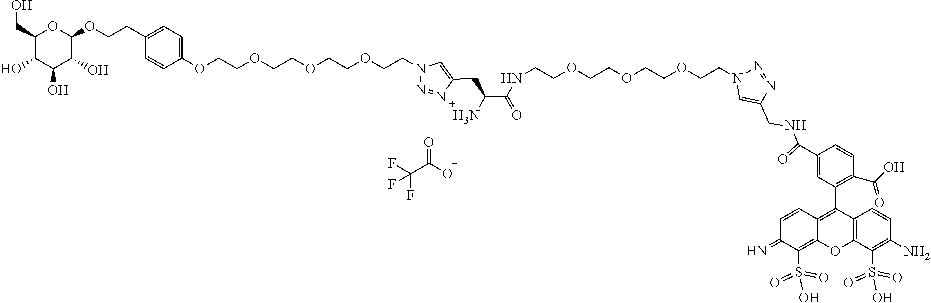 [SMILES: N=c1ccc2c(-c3cc(C(=O)NCc4cn(CCOCCOCCOCCNC(=O)[C@@H]([NH3+])Cc5cn(CCOCCOCCOCCOc6ccc(CCO[C@@H]7O[C@H](CO)[C@@H](O)[C@H](O)[C@H]7O)cc6)nn5)nn4)ccc3C(=O)O)c3ccc(N)c(S(=O)(=O)O)c3oc-2c1S(=O)(=O)O ; O=C([O-])C(F)(F)F]

FIM11 (3 mg, 2 μmol) was dissolved in a mixture of TFA (0.5 mL) and DCM (0.5 mL). After 45 minutes the reaction mixture was evaporated to dryness and was used without characterisation.

### Synthesis of I17

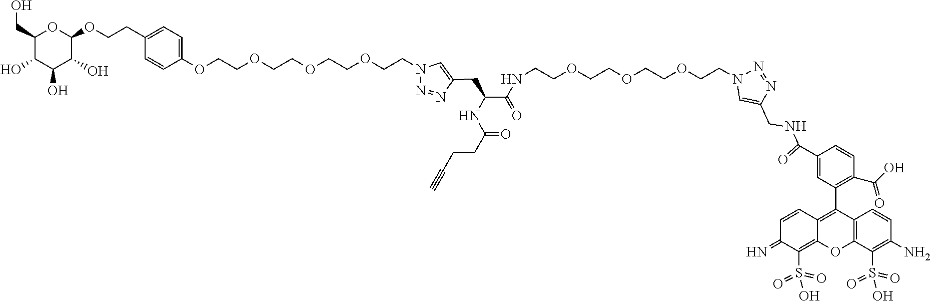 [SMILES: C#CCCC(=O)N[C@@H](Cc1cn(CCOCCOCCOCCOc2ccc(CCO[C@@H]3O[C@H](CO)[C@@H](O)[C@H](O)[C@H]3O)cc2)nn1)C(=O)NCCOCCOCCOCCn1cc(CNC(=O)c2ccc(C(=O)O)c(-c3c4ccc(=N)c(S(=O)(=O)O)c-4oc4c(S(=O)(=O)O)c(N)ccc34)c2)nn1]

Dissolved the crude 116 (3.0 mg, 2.0 μmol) in water (0.5 mL) containing NaHCO₃ (6.4 mg, 76 μmol). A stock solution of N-succinimidyl pent-4-ynoate (4.9 mg) in MeCN (1 mL) was made and portions (0.1 mL) of this were added with stirring and monitoring by LCMS over 24 hours until complete, where N-succinimidyl pent-4-ynoate (1.7 mg, 8.8 μmol) had been added. The reaction mixture was acidified with acetic acid (20 μL) and loaded directly onto a RP column and after freeze drying gave the product as an orange solid (2.2 mg, 1.5 μmol).

MS (electrospray, +ve), [M+H]⁺ calculated for C₆₄H₈₀N₁₁O₂₅S₂ requires: 1466.4673, found: 1466.4

### Synthesis of I18

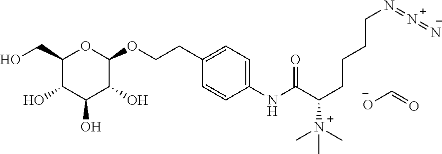

To a solution of I19 (15 mg, 0.033 mmol) in MeOH (1.00 mL) was added sodium carbonate (21 mg, 0.198 mmol) and methyl iodide (200 μL, 3.213 mmol). Reaction stirred overnight at RT before solvents were removed under vacuum. Purified by RP MPLC (H₂O with increasing MeCN (0.1% formic acid)). Product obtained as a white solid (13.0 mg, 0.024 mmol, 73%).

MS (electrospray, +ve) [M]⁺ calculated for C₂₃H₃₈N₅O₇ requires: 496.2766, found: 496.3.

### Synthesis of I19

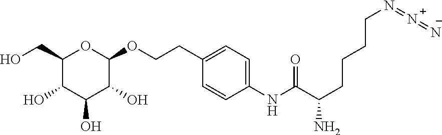 [SMILES: [N-]=[N+]=NCCCC[C@H](N)C(=O)Nc1ccc(CCO[C@@H]2O[C@H](CO)[C@@H](O)[C@H](O)[C@H]2O)cc1]

To a suspension of I20 (150 mg, 0.222 mmol) in DCM (3.00 mL) was added DBU (0.199 mL, 1.332 mmol). Reaction stirred for 30 minutes at RT before solvents were removed under vacuum. Purified by RP MPLC (H₂O with increasing MeCN). Product obtained as a pale-yellow oil (91 mg, 0.201 mmol, 90%).

MS (electrospray, +ve) [2M+H]⁺ calculated for C₄₀H₆₃N₁₀O₁₄ requires: 907.4520, found: 907.4.

### Synthesis of I20

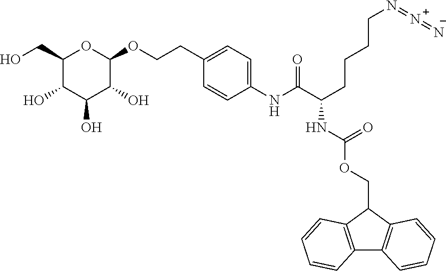 [SMILES: [N-]=[N+]=NCCCC[C@H](NC(=O)OCC1c2ccccc2-c2ccccc21)C(=O)Nc1ccc(CCO[C@@H]2O[C@H](CO)[C@@H](O)[C@H](O)[C@H]2O)cc1]

I25 (100 mg, 0.334 mmol), Fmoc-Lys(N3)—OH (145 mg, 0.367 mmol) and HATU (162 mg, 0.501 mmol) were dried under high vacuum for 1 hour before anhydrous DMF (1.0 mL) and DIPEA (87 μL, 0.501 mmol) were added. Reaction stirred overnight at RT before solvents were removed under vacuum. Purified by MPLC (EtOAc with increasing MeOH) and RP MPLC (H₂O with increasing MeCN). Product obtained as a white solid (150 mg, 0.222 mmol, 66%).

MS (electrospray, +ve) [M+H]⁺ calculated for C₃₅H₄₂N₅O₉ requires: 676.2977, found: 676.3.

### Synthesis of I21

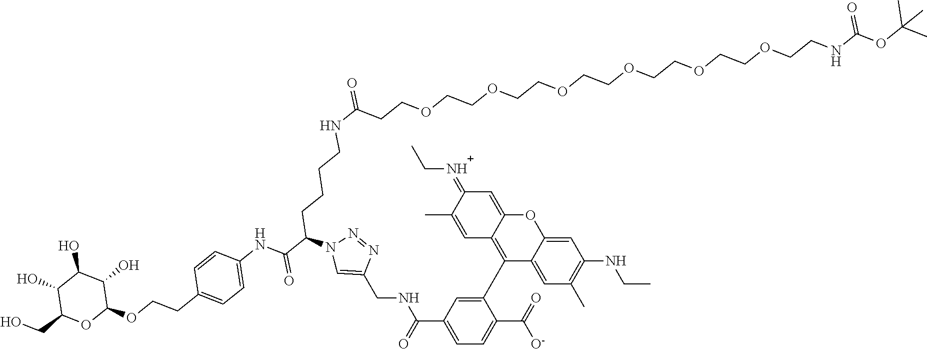 [SMILES: CCNc1cc2oc3c/c(=[NH+]/CC)c(C)cc-3c(-c3cc(C(=O)NCc4cn([C@H](CCCCNC(=O)CCOCCOCCOCCOCCOCCOCCNC(=O)OC(C)(C)C)C(=O)Nc5ccc(CCO[C@H]6O[C@@H](CO)[C@H](O)[C@@H](O)[C@@H]6O)cc5)nn4)ccc3C(=O)[O-])c2cc1C]

Prepared according to General Procedure B from I30 (15.0 mg, 0.017 mmol), R6G alkyne (5.0 mg, 0.010 mmol), CuSO₄·5H₂O (2.5 mg, 0.010 mmol), sodium ascorbate (15.0 mg, 0.076 mmol) and NaHCO₃ (5.0 mg, 0.060 mmol) in THF (1 mL) and water (1 mL). Purified by RP MPLC (H₂O with increasing MeCN (0.1% formic acid)). Product obtained as an orange solid (12.0 mg, 0.009 mmol, 86%).

MS (electrospray, +ve), [M+H]⁺ calculated for C₇₀H₉₈N₉O₂₀ requires: 1384.6923, found: 1384.7.

### Synthesis of I22

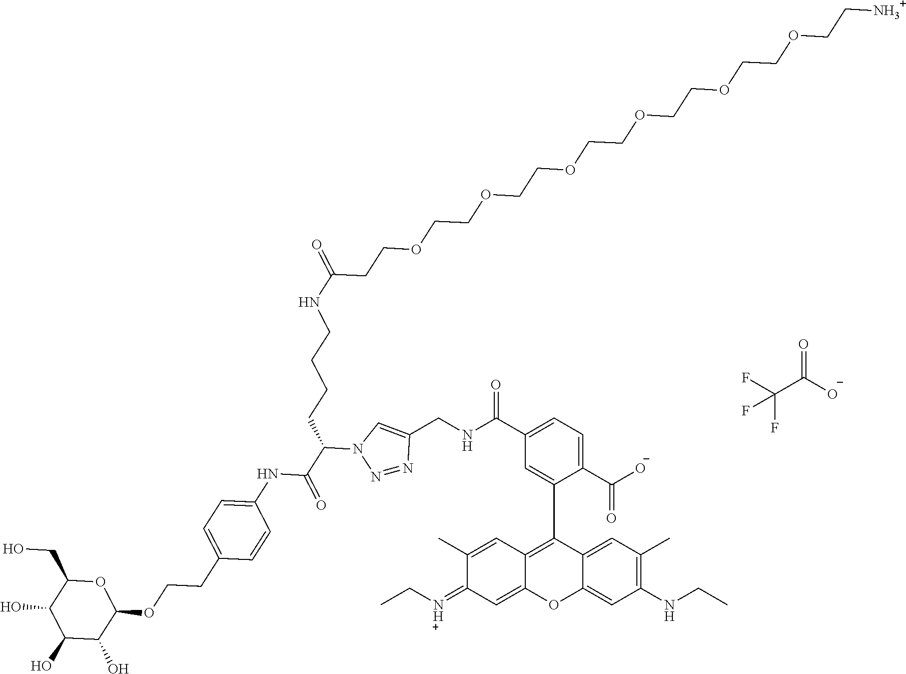 [SMILES: CCNc1cc2oc3c/c(=[NH+]/CC)c(C)cc-3c(-c3cc(C(=O)NCc4cn([C@@H](CCCCNC(=O)CCOCCOCCOCCOCCOCCOCC[NH3+])C(=O)Nc5ccc(CCO[C@@H]6O[C@H](CO)[C@@H](O)[C@H](O)[C@H]6O)cc5)nn4)ccc3C(=O)[O-])c2cc1C ; O=C([O-])C(F)(F)F]

Prepared according to General Procedure D from I21 (12 mg, 0.009 mmol) in TFA (0.5 mL) and DCM (1.2 mL). Purified by RP MPLC (H₂O with increasing MeCN (0.1% formic acid)). Product obtained as an orange solid (10 mg, 0.007 mmol, 83%).

MS (electrospray, +ve), [M+H]⁺ calculated for C₆₅H₉₀N₉O₁₈ requires: 1284.6399, found: 1284.6.

### Synthesis of I23

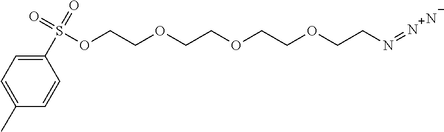

In a dried Schlenk flask, 2-(2-(2-(2-azidoethoxy)ethoxy)ethoxy)ethan-1-ol (4.860 g, 0.022 mol) was dissolved in anhydrous DCM (55. mL). TsCI (4.649 g, 0.024 mol) was then added in one portion followed by the addition of DMAP (2.979 g, 0.024 mol). The reaction was left stirring overnight at RT. The reaction was quenched by the addition of 1 M HCl and the resulting aqueous phase was washed with DCM. The organic layers were combined, dried over MgSO₄, filtered, and the resulting filtrate was absorbed onto SiO₂ for MPLC purification (DCM with increasing EtOAc). Product obtained as a slightly opaque colourless liquid (6.56 g, 0.018 mol, 79%).

¹H NMR (400 MHz, Chloroform-d) δ 7.81-7.65 (m, 2H), 7.33-7.22 (m, 2H), 4.17-4.04 (m, 2H), 3.64-3.49 (m, 13H), 3.31 (t, J=5.1 Hz, 2H), 2.38 (s, 3H).

### Synthesis of I24

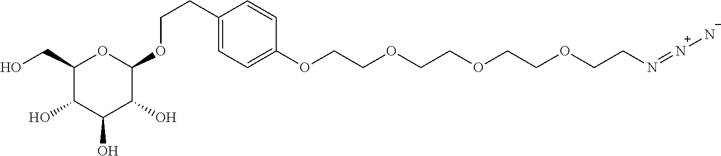 [SMILES: [NH-][NH+]=NCCOCCOCCOCCOc1ccc(CCO[C@@H]2O[C@H](CO)[C@@H](O)[C@H](O)[C@H]2O)cc1]

Salidroside (1.5 g, 4.995 mmol) was dissolved in DMF (24 mL). To this solution, Cs₂CO₃ (3.255 g, 9.99 mmol) was added followed by the addition of I23 (1.989 g, 5.328 mmol). The reaction was left stirring at 90° C. for 2 hours, left at RT overnight. The volatiles were blown under N2 flow and purified by SiO₂ MPLC (DCM with increasing MeOH). Product obtained (2.0 g, 3.988 mmol, 80%) with a 20% impurity of unreacted I23.

¹H NMR (400 MHz, Methanol-d₄) δ 7.18-7.11 (m, 2H), 6.87-6.78 (m, 2H), 4.27 (d, J=7.8 Hz, 1H), 4.10-4.06 (m, 2H), 3.84-3.55 (m, 15H), 3.31-3.13 (m, 5H), 2.85 (td, J=7.4, 7.0, 1.7 Hz, 2H).

### Synthesis of I25

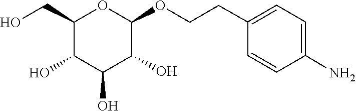 [SMILES: Nc1ccc(CCO[C@@H]2O[C@H](CO)[C@@H](O)[C@H](O)[C@H]2O)cc1]

To a slurry of Pd/C (0.101 g, 0.949 mmol, 10% w/w) in water (2.0 mL) the 134 (592 mg, 1.80 mmol) was added as a solid and dissolved in EtOH (14 mL). The reaction was stirred at RT under H₂ atmosphere for 4.5 hours. The mixture was filtered through celite and washed with MeOH. Evaporation of the volatiles yielded the product as a white solid (420 mg, 1.40 mmol, 78%).

¹H NMR (400 MHz, DMSO-d₆) δ 6.91-6.85 (m, 2H), 6.50-6.44 (m, 2H), 4.96 (d, J=5.0 Hz, 1H), 4.92 (d, J=4.8 Hz, 1H), 4.88 (d, J=5.0 Hz, 1H), 4.84 (s, 2H), 4.48 (t, J=5.9 Hz, 1H), 4.15 (d, J=7.8 Hz, 1H), 3.83 (td, J=9.1, 6.7 Hz, 1H), 3.65 (ddd, J=11.9, 6.0, 1.9 Hz, 1H), 3.52 (td, J=9.2, 6.4 Hz, 1H), 3.46-3.38 (m, 1H), 3.16-2.98 (m, 3H), 2.94 (ddd, J=8.9, 7.8, 4.9 Hz, 1H), 2.72-2.61 (m, 2H).

MS (electrospray, +ve), [M+Na]⁺ calculated for C₁₄H₂₁NNaO₆ requires: 322.1262, found: 322.1.

### Synthesis of I26

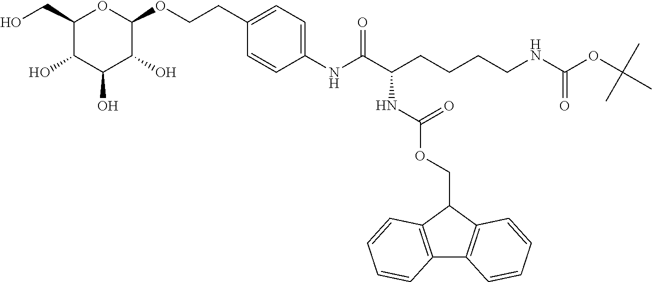 [SMILES: CC(C)(C)OC(=O)NCCCC[C@H](NC(=O)OCC1c2ccccc2-c2ccccc21)C(=O)Nc1ccc(CCO[C@@H]2O[C@H](CO)[C@@H](O)[C@H](O)[C@H]2O)cc1]

Fmoc-Lys(Boc)-OH (0.579 g, 1.231 mmol), HBTU (0.699 g, 1.843 mmol), and HOBt·H₂O (0.343 g, 2.238 mmol) dissolved in THF (15.0 mL) and DIPEA (0.780 mL, 4.477 mmol). The reaction was stirred at RT for 30 mins. After, 125 (0.335 g, 1.119 mmol) was added as a THF solution (2 mL), and the reaction was stirred at RT for 3 hours. Volatiles removed under vacuum and purified twice by RP MPLC (H₂O with increasing MeCN (0.1% formic acid)). Product redissolved in DCM to obtain a crispy foam (0.480 g, 0.640 mmol, 57%).

¹H NMR (400 MHz, DMSO-d₆): δ 9.91 (s, 1H), 7.86 (d, J=7.6 Hz, 2H), 7.70 (dd, J=7.6, 4.6 Hz, 2H), 7.58 (d, J=7.9 Hz, 1H), 7.50-7.43 (m, 2H), 7.43-7.33 (m, 2H), 7.29 (tdd, J=7.5, 4.1, 1.2 Hz, 2H), 7.22-7.12 (m, 2H), 6.75 (t, J=5.8 Hz, 1H), 4.99-4.79 (m, 3H), 4.46 (s, 1H), 4.29-3.98 (m, 6H), 3.87 (q, J=8.0 Hz, 1H), 3.59 (td, J=16.5, 6.5 Hz, 3H), 3.13-2.96 (m, 4H), 2.98-2.81 (m, 4H), 2.77 (d, J=8.1 Hz, 2H), 1.59 (d, J=7.1 Hz, 2H), 1.32 (s, 9H).

MS (electrospray, +ve), [M+Na]⁺ calculated for C₄₀H₅₁N₃NaO₁₁ requires: 772.3416, found: 772.3.

### Synthesis of I27

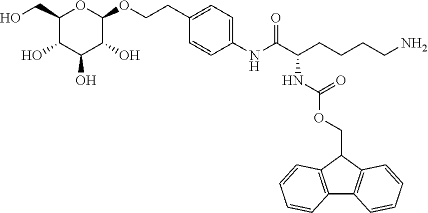 [SMILES: NCCCC[C@H](NC(=O)OCC1c2ccccc2-c2ccccc21)C(=O)Nc1ccc(CCO[C@@H]2O[C@H](CO)[C@@H](O)[C@H](O)[C@H]2O)cc1]

I26 (485.0 mg, 0.647 mmol) was dissolved in DCM (8.1 mL) and TFA (8.1 mL) and stirred at RT for 2 hours. Removed volatiles under flow of N₂, redissolved in DCM and removed under N₂ to give crude residue, which was used without further purification in the next step.

MS (electrospray, +ve), [M+H]⁺ calculated for C₃₅H₄₄N₃O₉ requires: 650.3073, found: 650.3.

### Synthesis of I28

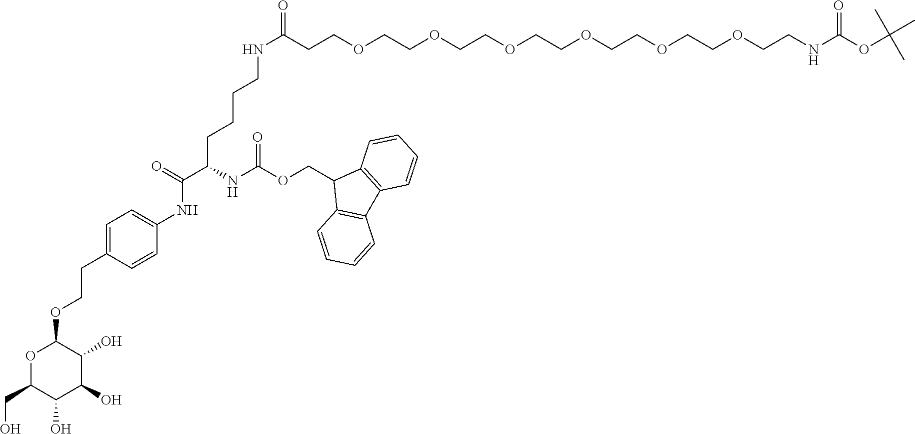 [SMILES: CC(C)(C)OC(=O)NCCOCCOCCOCCOCCOCCOCCC(=O)NCCCC[C@H](NC(=O)OCC1c2ccccc2-c2ccccc21)C(=O)Nc1ccc(CCO[C@@H]2O[C@H](CO)[C@@H](O)[C@H](O)[C@H]2O)cc1]

21-(Boc-amino)-4,7,10,13,16,19-hexaoxaheneicosanoic acid (0.352 g, 0.776 mmol), HBTU (0.294 g, 0.776 mmol), and HOBt·H₂O (0.198 g, 1.293 mmol) dissolved in THF (10.0 mL) and DIPEA (0.450 mL, 2.586 mmol). The reaction was stirred at RT for 30 mins. After, I27 (0.420 g, 0.646 mmol) was added as a THF solution (2 mL), and the reaction was stirred at RT for 2 hours. Volatiles removed under vacuum and purified by RP MPLC (H₂O with increasing MeCN (0.1% formic acid)). Product redissolved in DCM to obtain a crispy foam (0.479 g, 0.441 mol, 68%).

¹H NMR (400 MHz, DMSO-d₆) δ 9.91 (s, 1H), 7.86 (d, J=7.6 Hz, 2H), 7.79 (s, 1H), 7.70 (t, J=6.5 Hz, 2H), 7.57 (d, J=7.9 Hz, 1H), 7.46 (d, J=8.2 Hz, 2H), 7.38 (t, J=7.6 Hz, 2H), 7.29 (q, J=7.1 Hz, 2H), 7.16 (d, J=8.2 Hz, 2H), 6.71 (s, 1H), 4.99-4.81 (m, 3H), 4.45 (t, J=5.9 Hz, 1H), 4.31-3.98 (m, 5H), 3.86 (t, J=8.1 Hz, 1H), 3.68-3.49 (m, 4H), 3.18-2.70 (m, 13H), 2.24 (t, J=6.5 Hz, 2H), 1.61 (s, 3H), 1.33 (s, 9H).

MS (electrospray, +ve), [M+H]⁺ calculated for C₅₅H₈₁N₄O₁₈ requires: 1085.5541, found: 1085.5.

### Synthesis of I29

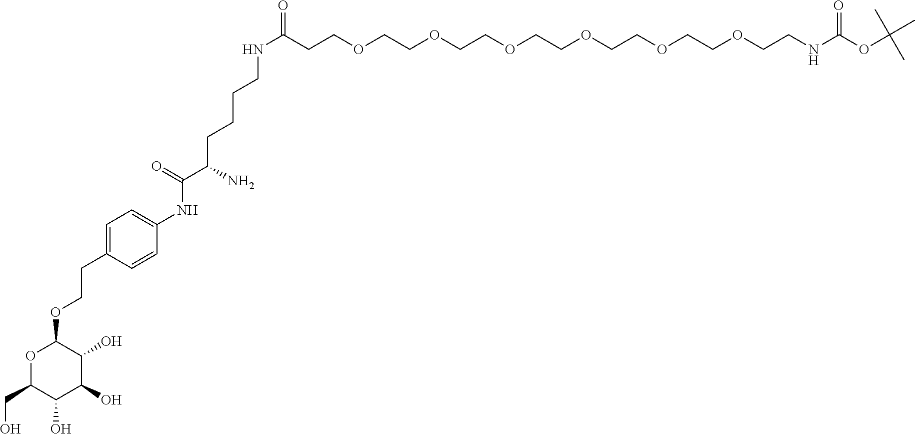 [SMILES: CC(C)(C)OC(=O)NCCOCCOCCOCCOCCOCCOCCC(=O)NCCCC[C@H](N)C(=O)Nc1ccc(CCO[C@@H]2O[C@H](CO)[C@@H](O)[C@H](O)[C@H]2O)cc1]

I28 (0.479 g, 0.441 mmol) dissolved in THF (4.4 mL). Added DBU (0.099 mL, 0.662 mmol) and the reaction was stirred at RT for 30 mins. Removed solvent under vacuum and purified by RP MPLC (H₂O with increasing MeCN (0.1% formic acid)). Product redissolved in DCM to obtain a crispy foam (0.278 g, 0.322 mol, 73%).

¹H NMR (400 MHz, DMSO-d₆) δ 8.18 (s, 1H), 7.78 (t, J=5.6 Hz, 1H), 7.48 (d, J=8.2 Hz, 2H), 7.17 (d, J=8.3 Hz, 2H), 6.72 (s, 1H), 4.14 (d, J=7.8 Hz, 2H), 3.88 (d, J=8.6 Hz, 2H), 3.15-2.86 (m, 13H), 2.78 (d, J=7.8 Hz, 3H), 2.24 (t, J=6.5 Hz, 3H), 1.33 (s, 9H).

MS (electrospray, +ve), [M+H]⁺ calculated for C₄₀H₇₁N₄O₁₆ requires: 863.4860, found: 863.4.

### Synthesis of I30

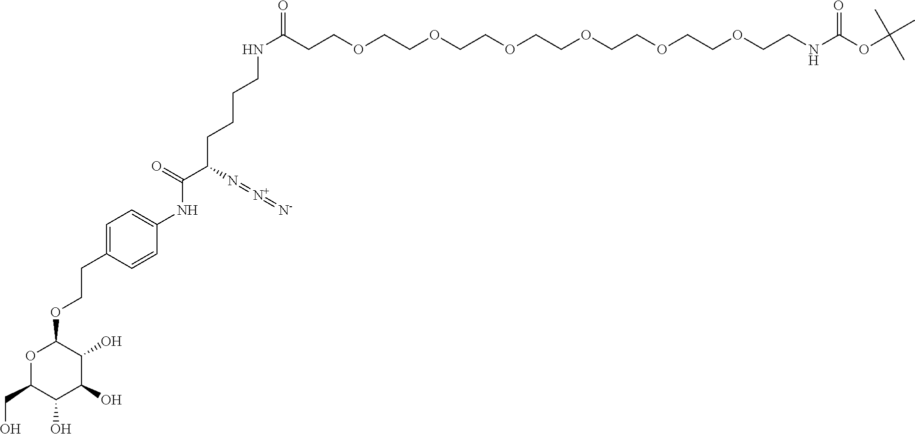 [SMILES: CC(C)(C)OC(=O)NCCOCCOCCOCCOCCOCCOCCC(=O)NCCCC[C@H](N=[N+]=[N-])C(=O)Nc1ccc(CCO[C@@H]2O[C@H](CO)[C@@H](O)[C@H](O)[C@H]2O)cc1]

I29 (278.0 mg, 0.322 mmol) and K₂CO₃ (133.6 mg, 0.966 mmol) dissolved in H₂O (6.4 mL) and MeOH (6.4 mL). Added imidazole-1-sulfonyl azide (81.0 mg, 0.387 mmol) and CuSO₄·5H₂O (16.1 mg, 0.064 mmol) as a solution in H₂O (1.5 mL). The reaction was left stirring at RT for 2 hours. Removed solvent under vacuum and purified by RP MPLC (H₂O with increasing MeCN (0.1% formic acid)). Product obtained as a colourless oil (280.0 mg, 0.315 mmol, 98%)

¹H NMR (400 MHz, DMSO-d₆) δ 10.11 (s, 1H), 8.12 (s, 1H), 7.80 (s, 1H), 7.46 (d, J=8.1 Hz, 2H), 7.19 (d, J=8.0 Hz, 2H), 6.71 (s, 1H), 4.87 (s, 4H), 4.14 (d, J=7.8 Hz, 2H), 3.96-3.74 (m, 5H), 3.14-2.69 (m, 11H), 2.24 (t, J=6.5 Hz, 2H), 1.75 (s, 3H), 1.40 (d, J=6.5 Hz, 2H), 1.33 (s, 9H).

MS (electrospray, +ve), [M+H]⁺ calculated for C₄₀H₆₉N₆O₁₆ requires: 889.4765, found: 889.4.

### Synthesis of I31

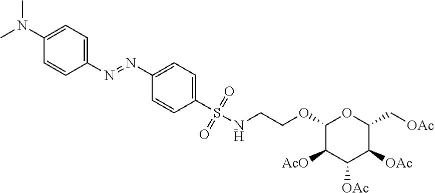 [SMILES: CC(=O)OC[C@H]1O[C@@H](OCCNS(=O)(=O)c2ccc(/N=N/c3ccc(N(C)C)cc3)cc2)[C@H](OC(C)=O)[C@@H](OC(C)=O)[C@@H]1OC(C)=O]

Dabsyl Chloride (53 mg, 0.164 mmol) dissolved in MeCN (2 mL) with DIPEA (0.060 mL, 0.344 mmol). 2-Aminoethyl 2,3,4,6-tetra-O-acetyl-β-D-glucopyranoside toluene-4-sulfonic acid salt (138 mg, 0.246 mmol) added, and reaction stirred for 16 hours. Quenched with sat. aq. NaHCO₃ (20 mL). Extracted with DCM (3×20 mL), organic solvents dried (Na₂SO₄) and concentrated under reduced pressure. Residue purified by MPLC (DCM with increasing EtOAc) to give the product as a brown solid (101 mg, 0.149 mmol, 91%).

¹H NMR (400 MHz, Chloroform-d) δ 8.34-7.81 (m, 6H), 6.97 (s, 2H), 5.20 (dd, J=10.0, 8.7 Hz, 1H), 5.08 (d, J=9.8 Hz, 1H), 4.95 (dd, J=9.6, 8.0 Hz, 1H), 4.48 (dd, J=8.0, 2.6 Hz, 1H), 4.29-4.15 (m, 2H), 3.93-3.79 (m, 1H), 3.79-3.66 (m, 2H), 3.32-3.21 (m, 4H), 3.20-3.13 (m, 2H), 3.05 (s, 2H), 2.10 (s, 3H), 2.08 (s, 3H), 2.03 (s, 3H), 2.01 (s, 3H).

### Synthesis of I32

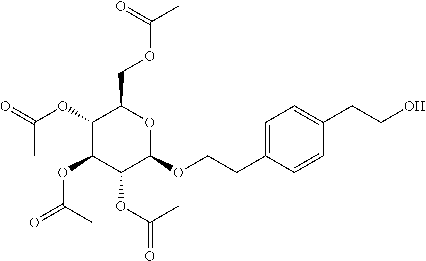 [SMILES: CC(=O)OC[C@H]1O[C@@H](OCCc2ccc(CCO)cc2)[C@H](OC(C)=O)[C@@H](OC(C)=O)[C@@H]1OC(C)=O]

To a suspension of silver carbonate (3.44 g, 12.48 mmol) and 1,4-bis(2-hydroxyethyl)benzene (1.04 g, 6.239 mmol) in DCM (40 mL) was added iodine (32 mg, 0.125 mmol). After stirring at RT for 10 min, acetobromo-α-D-glucose (2.82 g, 6.86 mmol) was added. After 1 h, reaction mixture was filtered through a bed of celite before solvent was removed under vacuum and purified by MPLC (DCM with increasing EtOAc). Product obtained as a colourless solid (1.36 g, 2.739 mmol, 44%).

¹H NMR (400 MHz, Chloroform-d) δ 7.1 (s, 4H), 5.2 (t, J=9.4 Hz, 1H), 5.1-5.0 (m, 1H), 5.0 (dd, J=9.5, 7.9 Hz, 1H), 4.5 (d, J=7.9 Hz, 1H), 4.3 (dd, J=12.3, 4.7 Hz, 1H), 4.2-4.1 (m, 2H), 3.8 (s, 2H), 3.7-3.6 (m, 2H), 2.9-2.8 (m, 4H), 2.1 (s, 3H), 2.0 (s, 3H), 2.0 (s, 3H), 1.9 (s, 3H).

### Synthesis of I33

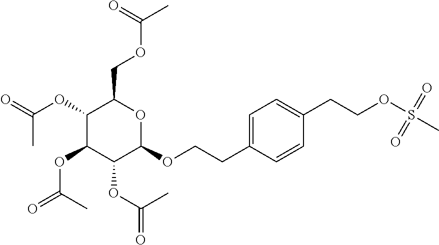 [SMILES: CC(=O)OC[C@H]1O[C@@H](OCCc2ccc(CCOS(C)(=O)=O)cc2)[C@H](OC(C)=O)[C@@H](OC(C)=O)[C@@H]1OC(C)=O]

To a solution of I32 (643 mg, 1.295 mmol) in THF (6.3 mL) at 0° C. was added DIPEA (0.36 mL, 2.073 mmol) and MsCl (0.12 mL, 1.555 mmol). After 20 min, reaction was diluted with EtOAc (10 mL) and washed successively with HCl (0.5 M, aq.), water, brine and then dried (Na₂SO₄). Solvents evaporated under reduced pressure to give a colourless oil (744 mg, 1.295 mmol, 99%).

¹H NMR (400 MHz, Chloroform-d) δ 7.1 (s, 4H), 5.2 (t, J=9.5 Hz, 1H), 5.1 (t, J=9.7 Hz, 1H), 5.0 (dd, J=9.6, 7.9 Hz, 1H), 4.5 (d, J=8.0 Hz, 1H), 4.4 (t, J=6.9 Hz, 2H), 4.3 (dd, J=12.3, 4.7 Hz, 1H), 4.2-4.1 (m, 2H), 3.7-3.6 (m, 2H), 3.0 (t, J=6.9 Hz, 2H), 2.9-2.8 (m, 5H), 2.1 (s, 3H), 2.0 (s, 2H), 2.0 (s, 3H), 2.0 (s, 3H).

### Synthesis of I34

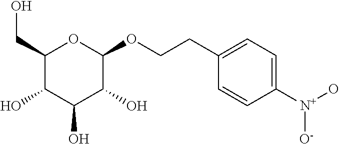 [SMILES: [O-][NH+](O)c1ccc(CCO[C@@H]2O[C@H](CO)[C@@H](O)[C@H](O)[C@H]2O)cc1]

A solution of 2-(4-Nitrophenyl)ethanol (14.0 g, 84 mmol) in dry DCM (80 mL) was stirred for 15 minutes with silver carbonate (15.4 g, 56 mmol) containing a few crystals of iodine. To this was added a solution of acetobromo-α-D-glucose (11.5 g, 28 mmol) in dry DCM (80 mL) and after 90 minutes the reaction mixture was filtered through a bed of celite and evaporated to give a viscous orange oil. The crude product was dissolved in MeOH (250 mL) and with rapid stirring solid sodium methoxide (1.51 g, 28 mmol) was added. After 45 minutes aqueous HCl (1 M, 10 mL) was added. Most of the solvent was evaporated then water (150 mL) was added and evaporated partially again to remove most of the MeOH.

The aqueous was diluted to300 mL and then washed with Et₂O (2×300 mL). The aqueous solution was concentrated and purified by RP MPLC to give the product as an off white crystalline solid (4.04 g, 12.3 mmol, 44% yield).

¹H NMR (400 MHz, DMSO-d₆) δ 8.1 (d, J=8.8 Hz, 1H), 7.6 (d, J=8.8 Hz, 1H), 5.0 (d, J=5.0 Hz, 1H), 4.9 (dd, J=12.4, 5.0 Hz, 2H), 4.5 (t, J=5.8 Hz, 1H), 4.2 (d, J=7.8 Hz, 1H), 4.0 (dt, J=10.0, 6.8 Hz, 1H), 3.7 (dt, J=10.0, 6.8 Hz, 1H), 3.7-3.6 (m, 1H), 3.2-3.1 (m, 2H), 3.1-3.0 (m, 3H), 2.9 (ddd, J=8.9, 7.8, 5.0 Hz, 1H).

### Synthesis of I35

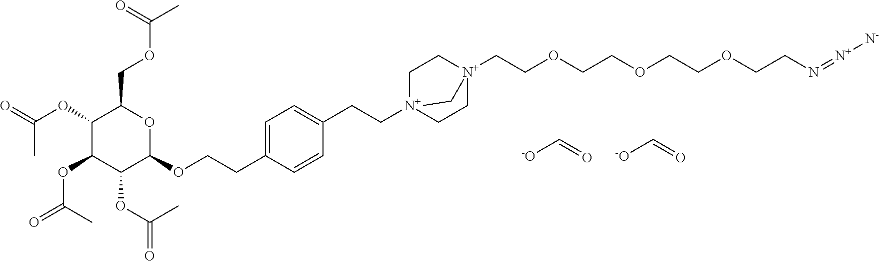

A solution of I33 (165 mg, 0.287 mmol) in EtOAc (0.65 mL) was added to DABCO (129 mg, 1.149 mmol), and left to stir at RT for 3 days. Et₂O (20 mL) was added to precipitate the product, which was isolated by centrifugation, as a white solid (190 mg, 0.277 mmol, 96%).

¹H NMR (400 MHz, Chloroform-d) δ 7.24 (d, J=7.8 Hz, 2H), 7.14 (d, J=7.8 Hz, 2H), 5.17 (t, J=9.4 Hz, 1H), 5.07 (t, J=9.6 Hz, 1H), 4.97 (dd, J=9.6, 7.9 Hz, 1H), 4.49 (d, J=7.9 Hz, 1H), 4.24 (dd, J=12.3, 4.7 Hz, 1H), 4.14-4.02 (m, 2H), 3.76-3.57 (m, 10H), 3.22 (t, J=7.5 Hz, 6H), 3.07 (dd, J=10.3, 6.4 Hz, 2H), 2.87-2.80 (m, 2H), 2.76 (s, 3H), 2.08 (s, 3H), 2.01 (s, 3H), 1.99 (s, 3H), 1.97 (s, 3H).

### Synthesis of I36

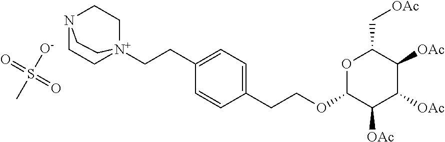

To a solution of I35 (57 mg, 0.083 mmol) in dry DMF (2 mL) was added I23 (218 mg, 0.584 mmol), NaHCO₃ (7 mg, 0.083 mmol) and NaI (12 mg, 0.080 mmol). Reaction was stirred at 80° C. for 2 days. Removed solvent under vacuum and purified by RP MPLC (H₂O with increasing MeCN (0.1% formic acid)). Product obtained as a white solid (15 mg, 0.017 mol, 20%).

MS (electrospray, +ve) [M−H]⁺ calculated for C₃₈H₅₈N₅O₁₃ requires: 792.4026, found: 792.4.

### Synthesis of I37

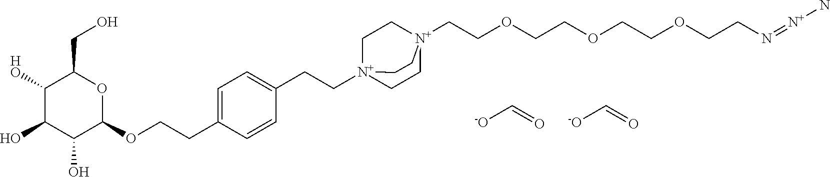

I36 (15 mg, 0.017 mmol) was dissolved in a solution of NaOH (2.0 mg, 0.051 mmol) in MeOH (0.30 mL) and stirred at RT for 1 hour. Reaction was acidified with formic acid before solvents were removed under vacuum. Purified by RP MPLC (H₂O with increasing MeCN (0.1% formic acid)). Product obtained as a white solid (8.0 mg, 0.011 mmol, 66%).

MS (electrospray, +ve) [M−H]⁺ calculated for C₃₀H₅₀N₅O₉ requires: 624.3603, found: 624.4.

### Synthesis of I38

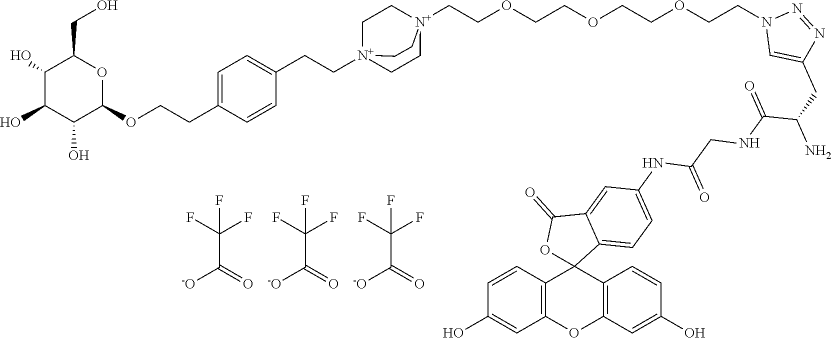

Prepared according to General Procedure D from FIM34 (2.5 mg, 0.002 mmol) in TFA (0.1 mL) and DCM (0.1 mL). Product obtained as an orange solid (10 mg, 0.008 mmol, 98%).

MS (electrospray, +ve) [M]²⁺ calculated for C₅₇H₇₂N₈O₁₆ requires: 562.2528, found: 562.3.

### Synthesis of I39

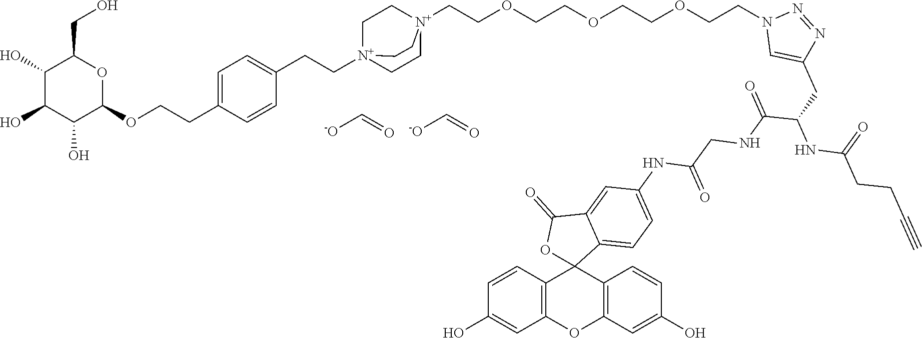

Prepared according to General Procedure C from I38 (2.8 mg, 0.002 mmol), N-succinimidyl pent-4-ynoate (1.9 mg, 0.010 mmol), NaHCO₃ (0.6 mg, 0.008 mmol) in MeCN (0.3 mL) and water (0.5 mL). Purified by RP MPLC (H₂O with increasing MeCN (0.1% formic acid)) to give the product as an orange solid (2.3 mg, 0.002 mmol, 93%).

MS (electrospray, +ve) [M]²⁺ calculated for C₆₂H₇₆N₈O₁₇ requires: 602.2659, found: 602.3.

### Synthesis of I40

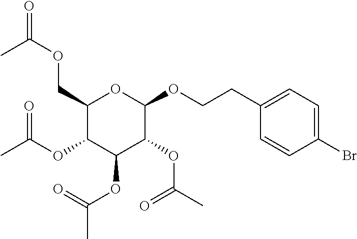 [SMILES: CC(=O)OC[C@H]1O[C@@H](OCCc2ccc(Br)cc2)[C@H](OC(C)=O)[C@@H](OC(C)=O)[C@@H]1OC(C)=O]

To a solution of acetobromo-α-D-glucose (24.914 g, 59.680 mmol) in dry DCM (˜400 mL) was added 2-(4-bromophenyl)ethanol (7.0 mL, 49.736 mmol) followed by silver carbonate (16.457 g, 59.683 mmol). After stirring for 17 hours the reaction mixture was filtered through celite and the filtrate evaporated to give a gum. This was dissolved in a mixture of acetic acid (50 mL) and water (10 mL). The mixture was heated to about 60° C. twice over the next hour. Then the solvent was removed under reduced pressure before adding acetic anhydride (40 mL) and pyridine (70 mL). After 2 hours at RT the solvent was removed then the gum was partitioned between EtOAc (200 mL) and water (200 mL). The organic washed with 1 M HCl, then water, then brine, dried over Na₂SO₄ and evaporated to give 35 g of a viscous orange oil. Dissolved in Et₂O and preadsorbed onto silica gel and partially purified by MPLC (petrol with increasing EtOAc).

Took all the product containing fractions and evaporated to give a gum (18.7 g). Made a solution of sodium hydroxide (0.6 g) in MeOH (100 mL) and added this to the gum. The gum slowly dissolved to give a fine slurry which after the addition of acetic acid (˜1 mL) dissolved the solid. Solvent was removed under vacuum and the gum was dissolved in water and purified by RP MPLC (H₂O with increasing MeOH) to give ˜5.8 g of deacetylated product.

Most of the deacetylated product (4.8 g, 13.2 mmol) was dissolved in a mixture of pyridine (50 mL, 627 mmol) and acetic anhydride (12 mL, 128 mmol) and after 6 hours the reaction mixture was evaporated to give a gum. The gum was then partitioned between EtOAc (200 mL) and water (200 mL). The organic washed with 1 M HCl, then water, then brine, dried (Na₂SO₄) which was evaporated to give a viscous oil that was crystallised from a mixture of Et₂O and petroleum ether 40/60. After drying the product was isolated as a white solid (6.54 g, 12.3 mmol, 25%).

¹H NMR (400 MHz, Chloroform-d) δ 7.4 (d, J=8.4 Hz, 2H), 7.1 (d, J=8.3 Hz, 2H), 5.2 (t, J=9.5 Hz, 1H), 5.1 (t, J=9.6 Hz, 1H), 5.0 (dd, J=9.4, 8.1 Hz, 1H), 4.2 (dd, J=12.3, 4.7 Hz, 1H), 4.1-4.1 (m, 2H), 3.7-3.6 (m, 2H), 2.8 (hept, J=8.3, 7.7 Hz, 2H), 2.1 (s, 3H), 2.0 (d, J=9.5 Hz, 6H), 1.9 (s, 3H).

### Synthesis of I41

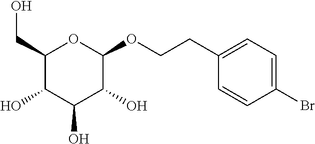 [SMILES: OC[C@H]1O[C@@H](OCCc2ccc(Br)cc2)[C@H](O)[C@@H](O)[C@@H]1O]

To a stirred suspension of I40 (3.010 g, 5.665 mmol) in MeOH (68 mL) was added sodium methoxide (0.282 g, 5.212 mmol). The reaction was evaporated to give a thick oil, then redissolved in MeOH and evaporated to give a foam. This was redissolved in more MeOH ˜15 mL and passed through a bed of regenerated H+ Dowex (10 g) washing through with MeOH. The solvent was removed which gave a white crystalline solid (2.03 g, 5.59 mmol 98% yield).

¹H NMR (400 MHz, DMSO-d₆) δ 7.5 (d, J=8.3 Hz, 1H), 7.2 (d, J=8.3 Hz, 2H), 4.5 (s, 1H), 4.2 (d, J=7.8 Hz, 1H), 3.9 (dt, J=9.9, 7.1 Hz, 1H), 3.7-3.6 (m, 2H), 3.4 (dd, J=11.7, 5.8 Hz, 1H), 3.2-3.0 (m, 2H), 3.1-3.0 (m, 1H), 2.9 (t, J=8.3 Hz, 1H), 2.8 (td, J=7.0, 1.9 Hz, 2H).

### Synthesis of I42

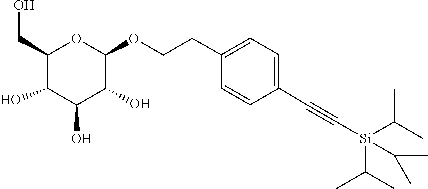 [SMILES: CC(C)[Si](C#Cc1ccc(CCO[C@@H]2O[C@H](CO)[C@@H](O)[C@H](O)[C@H]2O)cc1)(C(C)C)C(C)C]

I41 (2.022 g, 5.567 mmol), tetrakis(triphenylphosphine)palladium (0.643 g, 0.557 mmol) and copper iodide (0.106 g, 0.557 mmol) dissolved in dry THF (35 mL). To this was added (triisopropylsilyl)acetylene (6.2 mL, 27.836 mmol) followed by the Et₃N (7.0 mL, 95.3 mmol) and then the reaction mixture heated at 80° C. under nitrogen and shielded from light with aluminium foil.

After 20 hours of heating the reaction mixture was evaporated to give a thick suspension and then diluted with MeOH before evaporating to a viscous purple slurry. Added DCM and heated before filtering to remove the solids. The solution was purified by MPLC (DCM with increasing MeOH). Evaporation gave a white amorphous solid (2.17 g, 4.67 mmol, 84% yield).

### Synthesis of I43

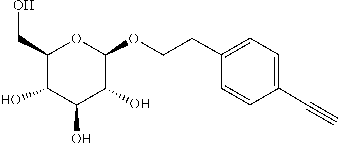 [SMILES: C#Cc1ccc(CCO[C@@H]2O[C@H](CO)[C@@H](O)[C@H](O)[C@H]2O)cc1]

I42 (1.050 g, 2.260 mmol) dissolved in THF (20 ml) with 7 drops of water and heated to t 40° C. Tetrabutylammonium fluoride (1.329 g, 5.084 mmol) added as a 1 M solution in THF (5 mL) which immediately gave a clear solution.

After 15 hours the reaction was evaporated to an oil then diluted with MeOH and preadsorbed onto silica gel (8 g) and dried to a fine powder. Purified by MPLC(DCM with increasing MeOH). Evaporation gave a solid which was shown by NMR to contain a small amount of tetrabutylammonium fluoride (728 mg (96% purity), 1.05 mmol, 100%).

¹H NMR (400 MHz, Methanol-d₄) δ 7.4 (d, J=8.3 Hz, 2H), 7.3 (d, J=8.4 Hz, 2H), 4.3 (d, J=7.8 Hz, 1H), 4.1 (dt, J=9.7, 7.2 Hz, 1H), 3.9 (dd, J=11.9, 1.8 Hz, 1H), 3.8 (dt, J=9.7, 7.0 Hz, 1H), 3.7-3.6 (m, 1H), 3.4 (s, 1H), 3.2 (dd, J=9.1, 7.8 Hz, 1H), 2.9 (t, J=7.1 Hz, 2H).

### Synthesis of I44

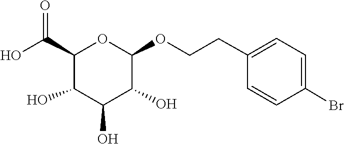 [SMILES: O=C(O)[C@H]1O[C@@H](OCCc2ccc(Br)cc2)[C@H](O)[C@@H](O)[C@@H]1O]

I41 (800.0 mg, 2.203 mmol) dissolved in a mixture of 0.5 M phosphate buffer (10 mL, pH 7) and MeCN (4 mL). To this solution was added TEMPO (90 mg, 0.573 mmol) which gave a yellow solution, followed by solid sodium chlorite (438 mg, 4.846 mmol). To this was added sodium hypochlorite (1% in water) in 1 mL portions every 10 minutes. 24 mL of sodium hypochlorite solution was used in total. MeCN evaporated and the remaining aqueous solution was washed with DCM (20 mL). The aqueous was then acidified with 4M HCl which gave a pH of about 2 and chlorine was generated. Half solution was loaded onto a 500 mg DVBPS reverse phase plug. This was washed with water before eluting the product from the plug using MeCN. This process was repeated with the second half and the solvent evaporated to give about 500 mg of product. The combined aqueous washings were reloaded onto the plug and the process repeated again. The product was combined, dissolved in water and lyophilised to give the product as a white solid (800 mg, 2.12 mmol, 96% yield).

¹H NMR (400 MHz, DMSO-d₆) δ 7.4 (d, J=8.3 Hz, 2H), 7.2 (d, J=8.3 Hz, 2H), 4.3 (d, J=7.8 Hz, 1H), 3.9 (dt, J=9.9, 7.0 Hz, 1H), 3.7-3.6 (m, 2H), 3.3 (t, J=9.3 Hz, 1H), 3.2 (t, J=9.0 Hz, 1H), 3.0 (dd, J=9.0, 7.8 Hz, 1H), 2.8 (t, J=7.0 Hz, 2H).

### Synthesis of I45

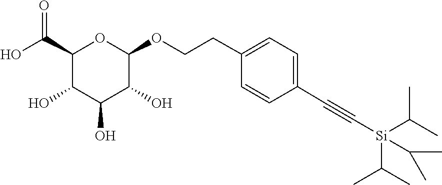 [SMILES: CC(C)[Si](C#Cc1ccc(CCO[C@@H]2O[C@H](C(=O)O)[C@@H](O)[C@H](O)[C@H]2O)cc1)(C(C)C)C(C)C]

I44 (168 mg, 0.445 mmol), tetrakis(triphenylphosphine)palladium (51.5 mg, 0.045 mmol) and copper iodide (8.5 mg, 0.045 mmol) were dissolved in dry THF (3 mL). To this was added (triisopropylsilyl)acetylene (0.500 mL, 2.227 mmol) followed by the Et₃N (0.558 mL, 7.57 mmol) and the reaction mixture was heated at 80° C. under nitrogen and shielded from light with aluminium foil.

After 5 hours solvent was removed under reduced pressure to give a dark gum and a thick oil. Added petrol which extracted away the near colourless oil and left the dark brown gum. The dark brown gum was dissolved in MeOH and passed through a PSDVB plug (0.5 g) eluting with MeOH. The solvent was evaporated, and the resultant light-yellow gum was dissolved in MeCN and water and acidified with 1 M HCl then purified by RP MPLC(H₂O with increasing MeCN (0.1% formic acid)) to give the product as a white solid (37 mg, 0.077 mmol, 17% yield).

¹H NMR (400 MHz, Methanol-d₄) δ 7.3 (d, J=8.2 Hz, 2H), 7.3 (d, J=8.2 Hz, 2H), 4.4 (d, J=7.6 Hz, 1H), 4.1 (d, J=8.7 Hz, 1H), 3.8 (d, J=9.4 Hz, 2H), 3.5 (s, 1H), 3.4 (t, J=8.6 Hz, 1H), 3.3-3.2 (m, 1H), 2.9 (t, J=6.9 Hz, 2H), 1.1 (d, J=2.6 Hz, 23H).

### Synthesis of I46

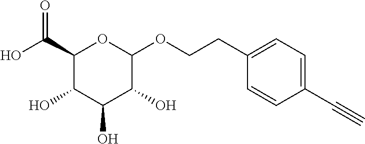 [SMILES: C#Cc1ccc(CCOC2O[C@H](C(=O)O)[C@@H](O)[C@H](O)[C@H]2O)cc1]

I45 (37.0 mg, 0.077 mmol) was dissolved in THF (1 mL) containing water (1 drop) to give a clear solution. To this was added tetrabutylammonium fluoride (0.3 mL of a 1 M solution) and the reaction mixture stirred overnight at RT. Worked up by diluting with MeOH and preadsorbing onto 2 g of C₁₈ silica and purifying by RP MPLC(H₂O with increasing MeCN (0.1% formic acid)). Evaporation gave a solid which was shown by NMR to contain tetrabutylammonium fluoride (40 mg (51% purity), 0.063 mmol, 82%). ¹H NMR (400 MHz, Methanol-d₄) δ 7.4 (d, J=8.3 Hz, 2H), 7.3 (d, J=8.3 Hz, 2H), 4.3 (d, J=7.8 Hz, 1H), 4.1 (dt, J=9.7, 7.1 Hz, 1H), 3.8 (dt, J=9.7, 7.2 Hz, 1H), 3.7 (d, J=9.6 Hz, 1H), 3.5 (t, J=9.3 Hz, 1H), 3.4-3.4 (m, 2H), 3.3-3.2 (m, 1H), 2.9 (t, J=7.1 Hz, 2H).

### Synthesis of I47

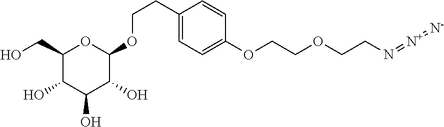 [SMILES: [N-]=[N+]=NCCOCCOc1ccc(CCO[C@@H]2O[C@H](CO)[C@@H](O)[C@H](O)[C@H]2O)cc1]

Salidroside (300 mg, 1.0 mmol) was dissolved in DMF (4.7 mL) and cesium carbonate (651 mg, 2.0 mmol) added. 148 (313 mg, 1.1 mmol) was added, and the reaction heated to 80° C. for 2 hours and then cooled to RT and stirred overnight. Purified by RP MPLC (H₂O with increasing MeCN (0.1% formic acid)) and solvent removed under vacuum to give the product (302 mg, 0.730 mmol, 73%) as a glassy solid.

1 H NMR (400 MHz, Methanol-d₄) δ 7.22-7.12 (m, 2H), 6.90-6.82 (m, 2H), 4.29 (d, J=7.8 Hz, 1H), 4.14-4.07 (m, 2H), 4.07-4.00 (m, 1H), 3.90-3.80 (m, 3H), 3.77-3.71 (m, 3H), 3.71-3.62 (m, 1H), 3.43-3.36 (m, 2H), 3.34 (dd, J=9.1, 1.6 Hz, 1H), 3.29-3.25 (m, 2H), 3.18 (dd, J=9.0, 7.8 Hz, 1H), 2.87 (ddd, J=8.3, 6.9, 1.8 Hz, 2H).

MS (electrospray, +ve), [M+Na]⁺ calculated for C₁₈H₂₇N₃O₈Na⁺ requires: 436.1690, found: 436.1.

### Synthesis of I48

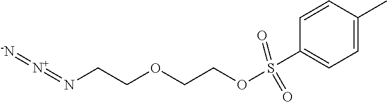

Diethylene glycol di(p-toluenesulfonate) (6.0 g, 14.476 mmol) was dissolved in DMF (12 mL) and NaN₃ (1.0 g, 15.561 mmol) was added. The reaction was stirred at RT for 2 days, then diluted with EtOAc (50 mL), filtered and the solvent removed under vacuum. The crude solid was dissolved in EtOAc, washed with water, brine and dried (Na₂SO₄) and the solvent removed under flow of nitrogen overnight. The crude oil was adsorbed onto silica gel and purified by RP MPLC (petrol with increasing Et₂O). The solvent was removed under vacuum to give the product (1.94 g, 6.80 mmol, 47%) as a colourless oil.

¹H NMR (400 MHz, Chloroform-d) δ 7.84-7.77 (m, 2H), 7.38-7.31 (m, 2H), 4.20-4.14 (m, 2H), 3.75-3.67 (m, 2H), 3.64-3.57 (m, 2H), 3.32 (dd, J=5.5, 4.4 Hz, 2H), 2.45 (s, 3H).

### Synthesis of I49

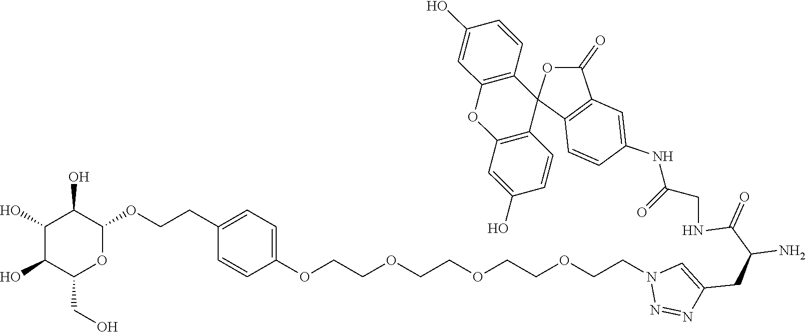 [SMILES: N[C@@H](Cc1cn(CCOCCOCCOCCOc2ccc(CCO[C@@H]3O[C@H](CO)[C@@H](O)[C@H](O)[C@H]3O)cc2)nn1)C(=O)NCC(=O)Nc1ccc2c(c1)C(=O)OC21c2ccc(O)cc2Oc2cc(O)ccc21]

I50 (102 mg, 0.093 mmol) was dissolved in DCM (2 mL) and trifluoroacetic acid (2 mL) added, and the reaction stirred at RT for 15 minutes. The solvent was removed under a flow of nitrogen, and the residue dissolved in water and purified by RP MPLC (H₂O with increasing MeCN (0.1% formic acid)), organic solvent removed under vacuum and the product was obtained by freeze drying the remaining aqueous to give an orange solid (89 mg, 0.089 mmol, 96%).

¹H NMR (400 MHz, DMSO-d₆) δ 10.49 (s, 1H), 8.51 (s, 1H), 8.33 (d, J=1.9 Hz, 1H), 7.91 (s, 1H), 7.86 (dd, J=8.4, 2.0 Hz, 1H), 7.22 (d, J=8.3 Hz, 1H), 7.18-7.11 (m, 2H), 6.85-6.79 (m, 2H), 6.67 (d, J=2.2 Hz, 2H), 6.58 (d, J=8.7 Hz, 2H), 6.54 (dd, J=8.7, 2.3 Hz, 2H), 4.93 (s, 1H), 4.47 (t, J=5.3 Hz, 2H), 4.16 (d, J=7.8 Hz, 1H), 4.05-3.95 (m, 4H), 3.88 (dt, J=9.8, 7.4 Hz, 1H), 3.80 (t, J=5.3 Hz, 2H), 3.72-3.58 (m, 4H), 3.56-3.47 (m, 9H), 3.18-2.99 (m, 4H), 2.94 (dd, J=9.0, 7.8 Hz, 1H), 2.86 (dd, J=14.6, 7.7 Hz, 1H), 2.82-2.73 (m, 2H).

MS (electrospray, +ve), [M+H]⁺ calculated for C₄₉H₅₇N₆O₁₇⁺ requires: 1001.3775, found: 1001.3.

### Synthesis of I50

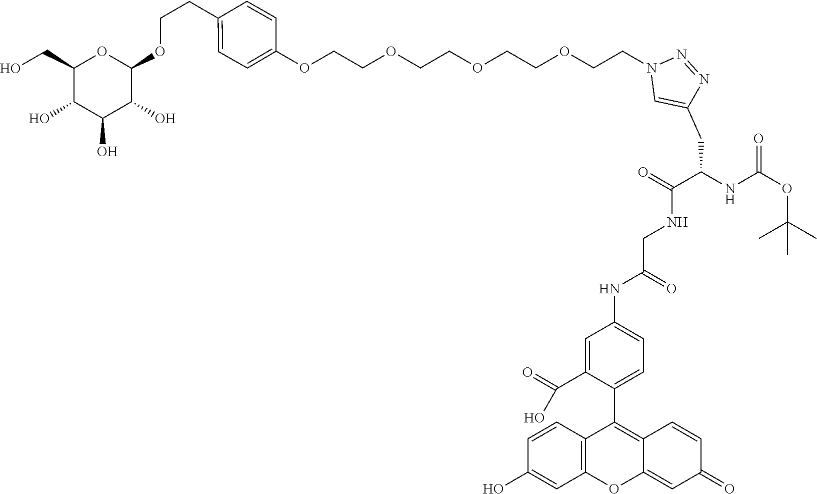 [SMILES: CC(C)(C)OC(=O)N[C@@H](Cc1cn(CCOCCOCCOCCOc2ccc(CCO[C@@H]3O[C@H](CO)[C@@H](O)[C@H](O)[C@H]3O)cc2)nn1)C(=O)NCC(=O)Nc1ccc(-c2c3ccc(=O)cc-3oc3cc(O)ccc23)c(C(=O)O)c1]

Prepared according to General Procedure B from I24 (54 mg, 0.108 mmol), I51 (61 mg, 0.102 mmol), Cu₂SO₄·5H₂O (5.1 mg, 0.020 mmol), sodium ascorbate (39.4 mg, 0.199 mmol) in THF (2 mL) and water (2 mL). Purified by RP MPLC (H₂O with increasing MeCN (0.1% formic acid)). Product obtained as an orange solid (102 mg, 0.093 mmol, 91%).

MS (electrospray, +ve), [M+H]⁺ calculated for C₅₄H₆₅N₆O₁₉ requires: 1101.4226, found: 1101.4

### Synthesis of I51

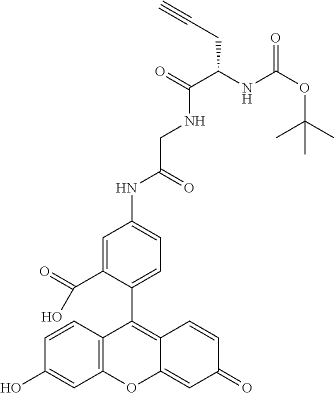 [SMILES: C#CC[C@H](NC(=O)OC(C)(C)C)C(=O)NCC(=O)Nc1ccc(-c2c3ccc(=O)cc-3oc3cc(O)ccc23)c(C(=O)O)c1]

Prepared according to General Procedure A from Boc-L-2-propargylglycine (213 mg, 0.615 mmol), I52 (226 mg, 0.559 mmol), HBTU (233 mg, 0.615 mmol), HOBt·H₂O (17.1 mg, 0.112 mmol) and DIPEA (0.29 mL, 1.67 mmol) in DMF (5 mL). Purified by RP MPLC (H₂O with increasing MeOH) to give the product as an orange solid (10 mg, 0.008 mmol, 54%).

¹H NMR (400 MHz, DMSO-d₆) δ 10.29 (s, 1H), 10.10 (s, 2H), 8.41 (t, J=5.7 Hz, 1H), 8.29 (dd, J=2.0, 0.6 Hz, 1H), 7.95 (s, 1H), 7.84 (dd, J=8.4, 2.0 Hz, 1H), 7.22 (dd, J=8.3, 0.6 Hz, 1H), 7.15 (d, J=8.1 Hz, 1H), 6.66 (d, J=2.3 Hz, 2H), 6.59 (dd, J=8.7, 1.2 Hz, 2H), 6.53 (dd, J=8.7, 2.3 Hz, 2H), 4.23-4.12 (m, 1H), 3.95 (d, J=5.7 Hz, 2H), 2.89 (d, J=0.5 Hz, 3H), 2.86 (t, J=2.6 Hz, 1H), 2.73 (d, J=0.7 Hz, 3H).

### Synthesis of I52

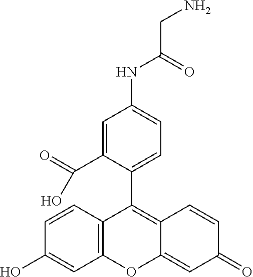 [SMILES: NCC(=O)Nc1ccc(-c2c3ccc(=O)cc-3oc3cc(O)ccc23)c(C(=O)O)c1]

I53 was dissolved in a solution of 20% piperidine in DMF (5 mL) and stirred at RT for 1.5 hours. Solvent removed under vacuum and water added to give a precipitate. The product was isolated by centrifugation and washing with DCM to give an orange solid that was dried under high vacuum (130 mg, 0.322 mmol, 375).

¹H NMR (400 MHz, DMSO-d₆) δ 8.32 (d, J=1.9 Hz, 1H), 7.88-7.79 (m, 1H), 7.16 (d, J=8.4 Hz, 1H), 6.62 (d, J=2.3 Hz, 2H), 6.55 (d, J=8.7 Hz, 2H), 6.50 (dd, J=8.7, 2.3 Hz, 2H).

¹H NMR signal for glycine CH₂ obscured by residual solvent signals.

### Synthesis of I53

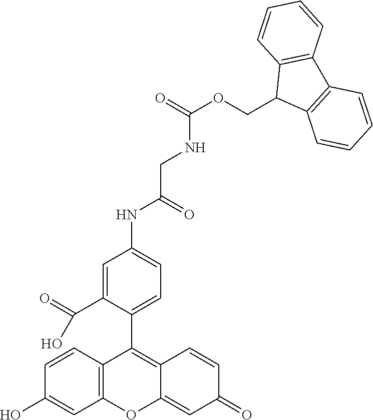 [SMILES: O=C(CNC(=O)OCC1c2ccccc2-c2ccccc21)Nc1ccc(-c2c3ccc(=O)cc-3oc3cc(O)ccc23)c(C(=O)O)c1]

Fmoc-glycine (400 mg, 1.345 mmol) was dissolved in THF (3 mL) and 1,1′-carbonyldiimidazole (218 mg, 1.345 mmol) added. The reaction was stirred for 45 minutes and fluoresceinamine isomer I (490 mg, 1.413 mmol) added, and the reaction stirred at RT overnight. The reaction mixture was partitioned between EtOAc (100 mL) and 1 M HCl (150 mL), the organic layer separated, washed with 1 M HCl (4×50 mL) and dried (Na₂SO₄). The solvent was removed under vacuum, adsorbed onto silica gel, and purified by MPLC (DCM with increasing EtOAc). The solvent was removed under vacuum to give the product (350 mg, 0.559 mmol, 42%) as an orange solid.

¹H NMR (400 MHz, Methanol-d₄) δ 8.29 (d, J=2.0 Hz, 1H), 7.83 (dd, J=8.4, 2.0 Hz, 1H), 7.77 (d, J=7.6 Hz, 2H), 7.66 (d, J=7.5 Hz, 2H), 7.36 (t, J=7.5 Hz, 2H), 7.31 (s, OH), 7.11 (d, J=8.3 Hz, 1H), 6.64 (d, J=2.5 Hz, 2H), 6.58 (d, J=8.7 Hz, 2H), 6.50 (dd, J=8.7, 2.4 Hz, 2H), 4.38 (d, J=7.0 Hz, 2H), 4.23 (t, J=7.0 Hz, 1H), 3.97 (s, 2H).

### Synthesis of I54

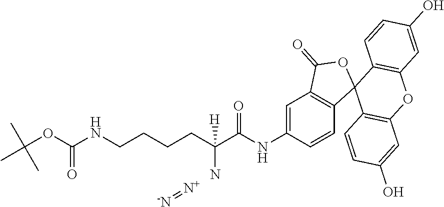

I55 (150 mg, 0.551 mmol), EDC·HCl (211 mg, 1.10 mmol) and HOBt·H₂O (168 mg, 1.10 mmol) were dissolved in DMSO (5.5 mL). DIPEA (0.384 mL, 2.20 mmol) was added and the reaction stirred at RT for 10 minutes. Fluoresceinamine isomer I (210 mg, 0.606 mmol) was added and the reaction stirred at 40° C. overnight. 1 M HCl was added to give a precipitate, which was collected by centrifugation and purified by RP MPLC (H₂O with increasing MeCN (0.1% formic acid)) to give the product (40 mg, 0.066 mmol, 12%) as an orange solid).

¹H NMR (400 MHz, Methanol-d₄) δ 8.35 (d, J=2.0 Hz, 1H), 7.91 (dd, J=8.4, 2.0 Hz, 1H), 7.18 (d, J=8.3 Hz, 1H), 6.74-6.65 (m, 2H), 6.61 (dd, J=8.7, 1.6 Hz, 2H), 6.54 (dd, J=8.7, 2.4 Hz, 2H), 3.97 (dd, J=7.6, 6.1 Hz, 1H), 3.07 (t, J=6.4 Hz, 2H), 2.01-1.88 (m, 2H), 1.59-1.47 (m, 3H), 1.45-1.39 (m, 12H).

MS (electrospray, +ve), [M+H]⁺ calculated for C₃₁H₃₂N₅O₈ requires: 601.2173, found: 601.2

### Synthesis of I55

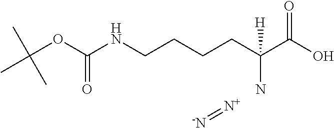

Nε-Boc-L-lysine (200 mg, 0.812 mmol) and K₂CO₃ (561 mg, 4.06 mmol) were dissolved in MeOH (16 mL) and water (16 mL). Added Imidazole-1-sulfonylazide hydrochloride (150 mg, 0.716 mmol) and then CuSO₄·5H₂O (27 mg, 0.108 mmol). Stirred at room temp for 1 hour and organic solvent removed under vacuum. Resultant aqueous solution acidified with 1 M HCl, and the product extracted with EtOAc. Solvent removed under vacuum to give the product (160 mg, 0.588 mmol, 72%) as a colourless oil.

¹H NMR (400 MHz, DMSO-d₆) δ 7.45 (s, OH), 6.79 (t, J=5.8 Hz, 1H), 4.06 (dd, J=8.3, 4.9 Hz, 1H), 2.89 (q, J=6.4 Hz, 2H), 1.77-1.65 (m, 1H), 1.65-1.54 (m, 1H), 1.36 (s, 14H).

### Synthesis of I56

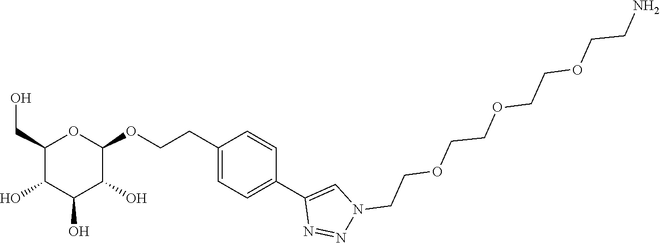 [SMILES: NCCOCCOCCOCCn1cc(-c2ccc(CCO[C@@H]3O[C@H](CO)[C@@H](O)[C@H](O)[C@H]3O)cc2)nn1]

Prepared according to General Procedure B from I43 (200 mg, 0.623 mmol), azido-PEG₃-amine (218 mg, 1.00 mmol), Cu₂SO₄·5H₂O (50 mg, 0.199 mmol), sodium ascorbate (129 mg, 0.654 mmol) in water (2 mL). Purified by RP MPLC (H₂O with increasing MeCN (0.1% formic acid)). Product obtained as a white solid (235 mg, 0.418 mmol, 67%).

¹H NMR (400 MHz, Methanol-d₄) δ 8.34 (s, 1H), 7.77-7.71 (m, 2H), 7.41-7.34 (m, 2H), 4.64 (dd, J=5.5, 4.5 Hz, 2H), 4.32 (d, J=7.8 Hz, 1H), 4.13 (dt, J=9.7, 7.1 Hz, 1H), 3.95 (dd, J=5.5, 4.5 Hz, 2H), 3.90-3.76 (m, 2H), 3.71-3.52 (m, 12H), 3.39-3.33 (m, 1H), 3.29-3.26 (m, 2H), 3.18 (dd, J=9.1, 7.8 Hz, 1H), 3.05-3.01 (m, 2H), 2.98 (t, J=7.0 Hz, 2H).

### Synthesis of I57

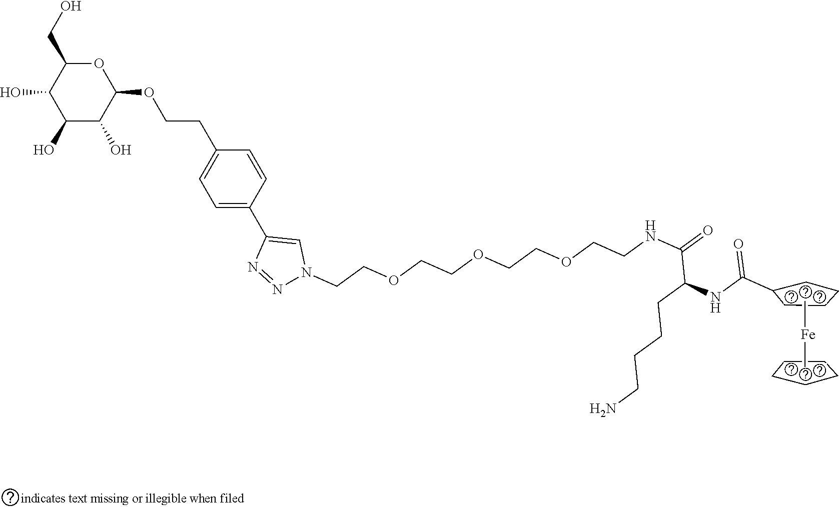

Prepared according to General Procedure D from I58 (43 mg, 0.045 mmol) in TFA (0.5 mL) and DCM (1.5 mL). Product obtained as an orange solid (39 mg, 0.045 mmol, 98%). Product was used without further purification and characterisation for the next step.

### Synthesis of I58

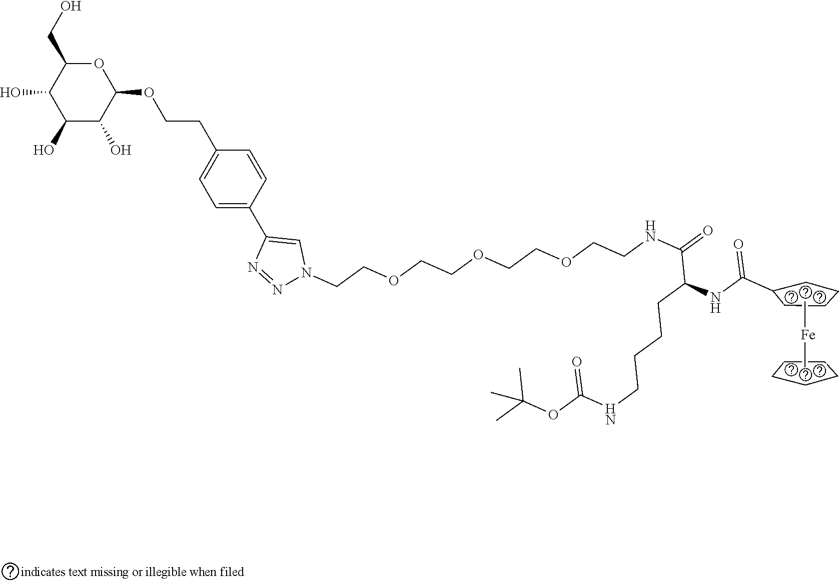

I59 (36 mg, 0.048 mmol) was dissolved in water (0.5 mL) and sodium bicarbonate (31 mg, 0.372 mmol) added. Added N-Succinimidyl ferrocenecarboxylate (26 mg, 0.081 mmol) and DMF (1 mL) and the reaction stirred at RT for 27 hours. Reaction mixture diluted with water (15 mL) and purified by RP MPLC (H₂O with increasing MeCN) to give the product (9 mg, 0.009 mmol, 20%) as an orange solid.

¹H NMR (400 MHz, Methanol-d₄) δ 8.32 (s, 1H), 7.73 (d, J=8.3 Hz, 1H), 7.36 (d, J=8.3 Hz, 2H), 4.62 (dd, J=5.5, 4.5 Hz, 2H), 4.49 (dd, J=9.1, 5.4 Hz, 1H), 4.40 (ddt, J=2.5, 1.6, 0.8 Hz, 2H), 4.32 (d, J=7.8 Hz, 1H), 4.21 (s, 4H), 4.12 (dt, J=9.6, 7.2 Hz, 1H), 3.93 (dd, J=5.6, 4.5 Hz, 2H), 3.87 (dd, J=11.8, 1.7 Hz, 1H), 3.79 (dt, J=9.7, 7.2 Hz, 1H), 3.70-3.64 (m, 1H), 3.64-3.57 (m, 4H), 3.56-3.49 (m, 4H), 3.47 (t, J=5.4 Hz, 2H), 3.39-3.32 (m, 3H), 3.30-3.26 (m, 2H), 3.20 (dd, J=9.0, 7.8 Hz, 1H), 3.04 (t, J=6.5 Hz, 2H), 2.97 (t, J=7.2 Hz, 2H), 1.89-1.68 (m, 2H), 1.57-1.43 (m, 4H), 1.40 (s, 9H).

### Synthesis of I59

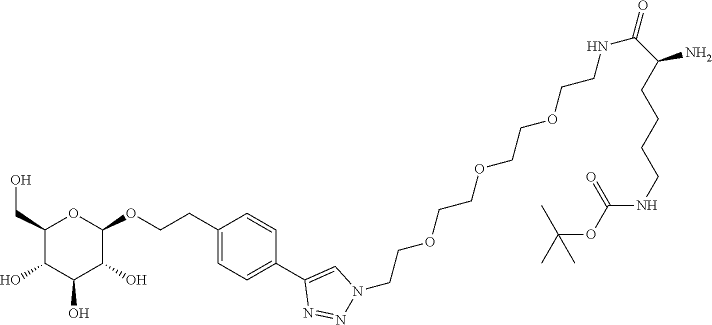 [SMILES: CC(C)(C)OC(=O)NCCCC[C@H](N)C(=O)NCCOCCOCCOCCn1cc(-c2ccc(CCO[C@@H]3O[C@H](CO)[C@@H](O)[C@H](O)[C@H]3O)cc2)nn1]

I60 (190 mg, 0.194 mmol) was dissolved in DMF (4 mL), piperidine (0.2 mL, 2.02 mmol) added and the reaction stirred at RT for 1 hour. The solvent removed under a flow of nitrogen and partitioned between DCM/MeOH (9:1) and water (neutralised to pH 7). The aqueous layer was collected and purified by RP MPLC (H₂O with increasing MeCN (0.1% formic acid)). The solvent was removed under vacuum to give the product (147 mg, 0.195 mmol, 98%) as a colourless gummy solid.

¹H NMR (400 MHz, Methanol-d₄) δ 8.38 (s, 1H), 8.33 (s, 1H), 7.74 (d, J=8.4 Hz, 2H), 7.40-7.34 (m, 2H), 4.63 (dd, J=5.6, 4.5 Hz, 2H), 4.32 (d, J=7.8 Hz, 1H), 4.13 (dt, J=9.6, 7.2 Hz, 1H), 3.95 (dd, J=5.6, 4.5 Hz, 2H), 3.87 (dd, J=11.8, 1.7 Hz, 1H), 3.83-3.74 (m, 2H), 3.71-3.58 (m, 5H), 3.57-3.47 (m, 4H), 3.45 (t, J=5.2 Hz, 2H), 3.42-3.32 (m, 3H), 3.29-3.25 (m, 2H), 3.19 (dd, J=9.1, 7.8 Hz, 1H), 3.00 (dt, J=15.6, 7.0 Hz, 4H), 1.87-1.73 (m, 2H), 1.50 (q, J=7.6, 7.2 Hz, 2H), 1.42 (s, 9H), 1.40-1.32 (m, 2H).

### Synthesis of I60

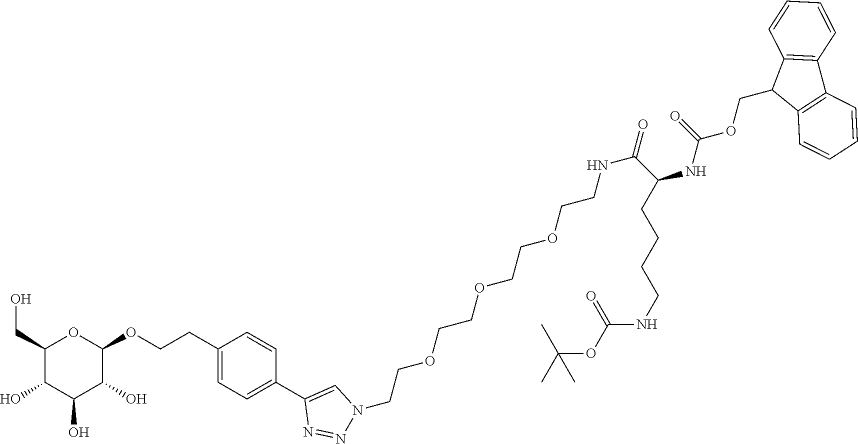 [SMILES: CC(C)(C)OC(=O)NCCCC[C@H](NC(=O)OCC1c2ccccc2-c2ccccc21)C(=O)NCCOCCOCCOCCn1cc(-c2ccc(CCO[C@@H]3O[C@H](CO)[C@@H](O)[C@H](O)[C@H]3O)cc2)nn1]

Prepared according to General Procedure A from I56 (164 mg, 0.291 mmol), Na-Fmoc-Nε-Boc-L-Iysine (163 mg, 0.350 mmol), HBTU (143 mg, 0.379 mmol), HOBt·H₂O (13.4 mg, 0.0.087 mmol) and DIPEA (0.15 mL, 0.874 mmol) in DMF (4 mL). Purified by RP MPLC (H₂O with increasing acetone) to give the product as a white solid (190 mg, 0.194 mmol, 67%).

¹H NMR (400 MHz, Methanol-d₄) δ 8.25 (s, 1H), 7.76 (d, J=7.5 Hz, 1H), 7.69 (d, J=8.2 Hz, 2H), 7.62 (dd, J=7.6, 4.1 Hz, 2H), 7.35 (td, J=7.6, 1.1 Hz, 2H), 7.31 (d, J=8.2 Hz, 2H), 7.27 (td, J=7.5, 1.2 Hz, 2H), 6.52 (s, 1H), 4.55 (t, J=5.0 Hz, 2H), 4.40-4.27 (m, 3H), 4.17 (t, J=6.8 Hz, 1H), 4.09 (dt, J=9.7, 7.3 Hz, 1H), 4.02 (dd, J=9.0, 5.3 Hz, 1H), 3.89-3.82 (m, 3H), 3.75 (dt, J=9.7, 7.2 Hz, 1H), 3.69-3.61 (m, 1H), 3.58-3.50 (m, 4H), 3.46 (h, J=3.1 Hz, 4H), 3.41 (t, J=5.4 Hz, 2H), 3.37-3.31 (m, 1H), 3.28-3.24 (m, 5H), 3.18 (dd, J=9.1, 7.8 Hz, 1H), 3.03-2.89 (m, 4H), 1.70 (p, J=6.5, 5.7 Hz, 1H), 1.64-1.52 (m, 1H), 1.39 (s, 9H), 1.36-1.20 (m, 2H).

### Synthesis of I61

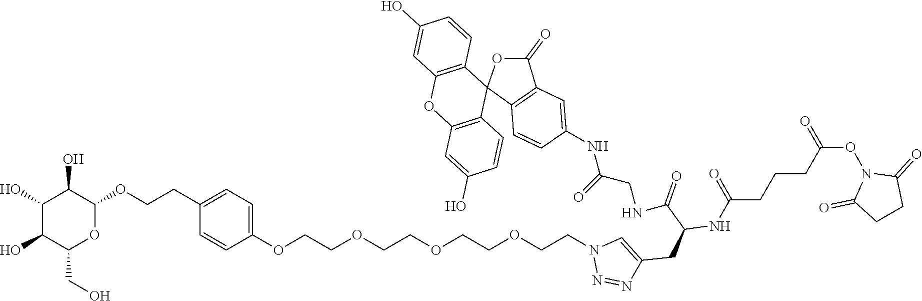 [SMILES: O=C(CNC(=O)[C@H](Cc1cn(CCOCCOCCOCCOc2ccc(CCO[C@@H]3O[C@H](CO)[C@@H](O)[C@H](O)[C@H]3O)cc2)nn1)NC(=O)CCCC(=O)ON1C(=O)CCC1=O)Nc1ccc2c(c1)C(=O)OC21c2ccc(O)cc2Oc2cc(O)ccc21]

I62 (5.9 mg, 5.3 μmol), EDC (11.0 mg, 7.8 μmol) and N-hydroxysuccinimide (12.2 mg, 106 μmol) dissolved in H₂O/MeCN (1.0 mL, 4:1) and stirred for 2 hours at RT. Solvents evaporated and residue purified by RP MPLC (H₂O with increasing MeCN) to give the product as an orange solid (4.5 mg, 2.6 μmol, 49%).

MS (electrospray, +ve) [M+H]⁺ calculated for C₅₈H₆₆N₇O₂₂ requires: 1212.4255, found: 1212.3.

### Synthesis of I62

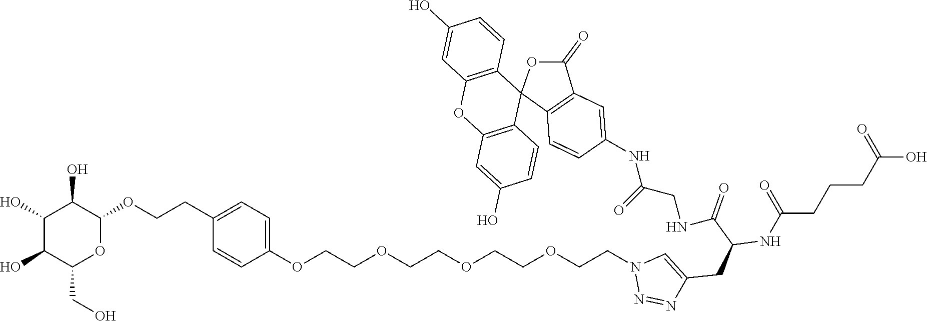 [SMILES: O=C(O)CCCC(=O)N[C@@H](Cc1cn(CCOCCOCCOCCOc2ccc(CCO[C@@H]3O[C@H](CO)[C@@H](O)[C@H](O)[C@H]3O)cc2)nn1)C(=O)NCC(=O)Nc1ccc2c(c1)C(=O)OC21c2ccc(O)cc2Oc2cc(O)ccc21]

Prepared according to General Procedure C from I49 (5.3 mg, 5.3 μmol), glutaric anhydride (3.3 mg, 29.1 μmol) and NaHCO₃ (7.6 mg, 90 μmol) in water (0.4 mL). Purified by RP MPLC (H₂O with increasing MeCN). Product obtained as an orange solid (5.9 mg, 5.3 μmol, 99%).

MS (electrospray, +ve) [M+H]⁺ calculated for C₅₄H₆₃N₆O₂₀ requires: 1115.4092, found: 1115.4.

### Synthesis of I63

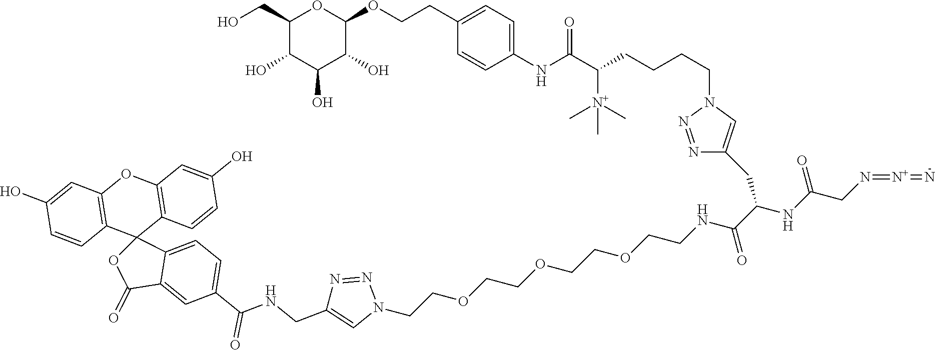 [SMILES: C[N+](C)(C)[C@@H](CCCCn1cc(C[C@H](NC(=O)CN=[N+]=N)C(=O)NCCOCCOCCOCCn2cc(CNC(=O)c3ccc4c(c3)C(=O)OC43c4ccc(O)cc4Oc4cc(O)ccc43)nn2)nn1)C(=O)Nc1ccc(CCO[C@@H]2O[C@H](CO)[C@@H](O)[C@H](O)[C@H]2O)cc1]

Prepared according to General Procedure C from I64 (2.3 mg, 1.6 μmol), azidoacetic acid NHS ester (1.6 mg, 7.8 μmol) and NaHCO₃ (0.4 mg, 4.8 μmol) in MeCN/water (0.6 mL, 2:1). Purified by RP MPLC (H₂O with increasing MeCN (0.1% formic acid)). Product obtained as an orange solid (2.0 mg, 1.5 μmol, 93%).

MS (electrospray, +ve) [M]⁺ calculated for C₆₂H₇₈N₁₄O₁₇ requires: 1305.5535, found: 1305.5.

### Synthesis of I64

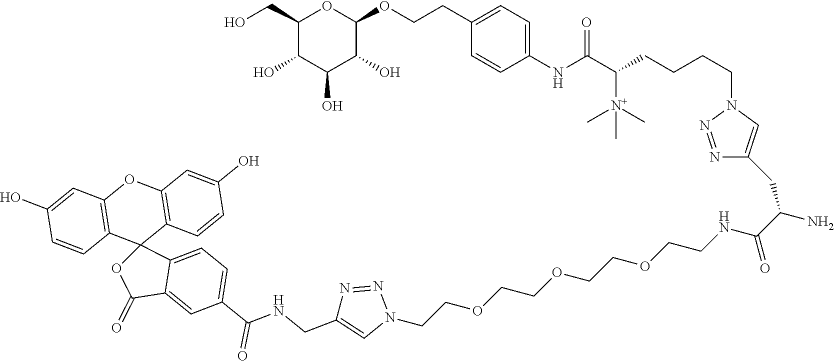 [SMILES: C[N+](C)(C)[C@@H](CCCCn1cc(C[C@H](N)C(=O)NCCOCCOCCOCCn2cc(CNC(=O)c3ccc4c(c3)C(=O)OC43c4ccc(O)cc4Oc4cc(O)ccc43)nn2)nn1)C(=O)Nc1ccc(CCO[C@@H]2O[C@H](CO)[C@@H](O)[C@H](O)[C@H]2O)cc1]

Prepared according to General Procedure D from FIM32 (2.2 mg, 1.6 μmol) in TFA (0.3 mL) and DCM (0.3 mL). Product obtained as an orange solid (2.3 mg, 1.6 mmol, 99%).

MS (electrospray, +ve) [M]⁺ calculated for C₅₇H₇₂N₈O₁₆ requires: 1222.5415, found: 1222.5.

### Synthesis of I65

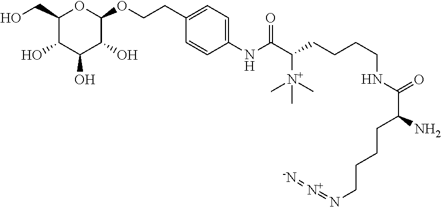

I66 (6 mg, 0.005 mmol) was dissolved in MeOH (7 mL) and 2 M NaOH (3 mL, aq.) added. The reaction was stirred at RT for 1 h, acidified with formic acid and the solvent removed under vacuum. The crude solid was purified by RP MPLC (water with increasing MeCN (0.1% formic acid)) to give the product (1.5 mg, 0.002 mmol, 46%) as a white solid.

MS (electrospray, +ve) [M]⁺ calculated for C₂₉H₅₀N₇O₈ requires: 624.3716, found: 624.3.

### Synthesis of I66

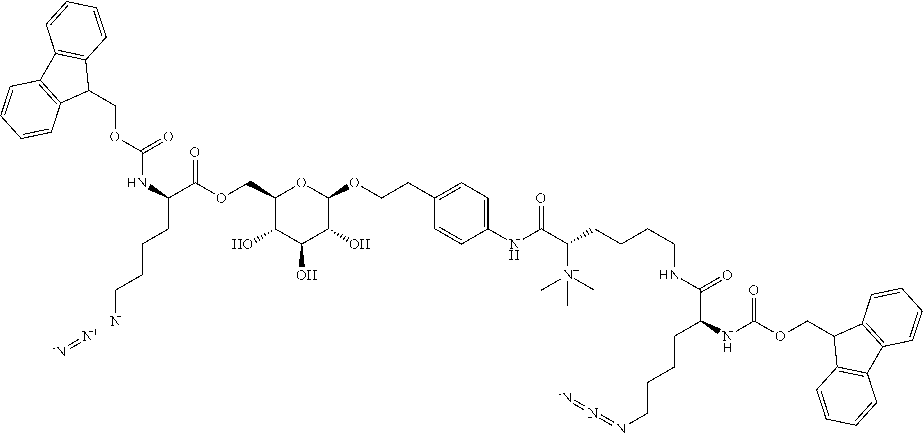

Prepared according to General Procedure A from I67 (18 mg, 0.032 mmol), Fmoc-azido-L-lysine (25.3 mg, 0.064 mmol), HBTU (36.5 g, 0.096 mmol) and DIPEA (0.02 mL, 0.096 mmol) in DMF (0.6 mL). Purified by RP MPLC (H₂O with increasing MeCN (0.1% formic acid)) to give the product as a white solid (6 mg, 0.005 mmol, 16%).

MS (electrospray, +ve) [M]⁺ calculated for C₆₅H₈₀N₁₁O₁₃ requires: 1222.5932, found: 1222.6.

### Synthesis of I67

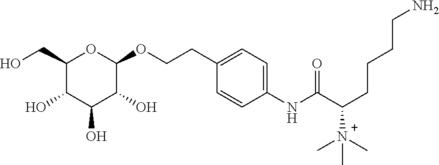 [SMILES: C[N+](C)(C)[C@@H](CCCCN)C(=O)Nc1ccc(CCO[C@@H]2O[C@H](CO)[C@@H](O)[C@H](O)[C@H]2O)cc1]

I18 (27 mg, 0.05 mmol) was dissolved in THF (0.4 mL) and water (0.4 mL) and sparged with nitrogen for 5 minutes. PMe₃ (1 M solution in THF, 0.15 mL, 0.15 mmol) was added and the reaction stirred at RT for 1.5 hours. Solvent and excess PMe₃ removed under flow for nitrogen, solvent removed under vacuum and the crude solid purified by RP MPLC (water with increasing MeCN (0.1% formic acid)). The product was lyophilised to give a white solid (18.5 mg, 0.033 mmol, 66%).

MS (electrospray, +ve) [M]⁺ calculated for C₂₃H₄₀N₃O₇ requires: 470.2861, found: 470.2.

### Synthesis of I68

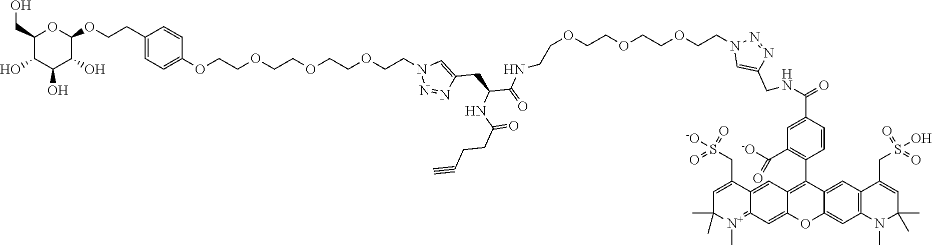

Dissolved I69 (2.2 mg, 1.4 μmol) and DIPEA (50 μL, 287 μmol) in DMF (0.5 mL). then, 4-Pentynoic acid succinimidyl ester (4 mg, 20.5 μmol) was added and the reaction mixture was stirred at RT overnight. Next morning, solvents were blown down and the crude redissolved in water and purified by RP MPLC (H₂O with increasing MeCN (0.1% formic acid)) and then RP HPLC to give the product as a blue solid (1 mg, 0.596 μmol, 43%).

MS (electrospray, +ve), [M+H]⁺ calculated for C₇₈H₉₉N₁₁O₂₅S₂ requires: 1654.822, found: 1654.5.

### Synthesis of I69

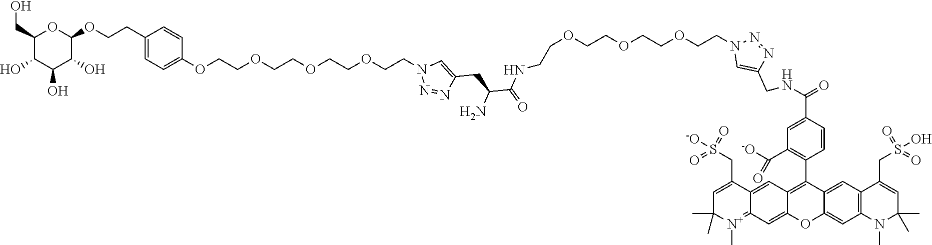

Prepared according to General Procedure D from FIM10 (2.5 mg, 1.4 μmol) in TFA (0.1 mL) and DCM (0.1 mL). After 1 hour, volatiles were blown down under N₂ flow. The obtained product was used without further purification and characterisation in the next step.

### Synthesis of I70

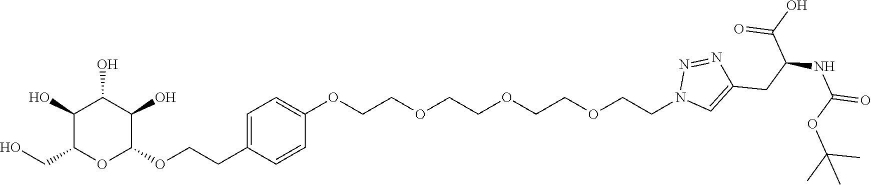 [SMILES: CC(C)(C)OC(=O)N[C@@H](Cc1cn(CCOCCOCCOCCOc2ccc(CCO[C@@H]3O[C@H](CO)[C@@H](O)[C@H](O)[C@H]3O)cc2)nn1)C(=O)O]

Prepared according to General Procedure B from I24 (219 mg, 0.437 mmol), Boc-L-2-propargylglycine (157 mg, 0.736 mmol), sodium ascorbate (35 mg, 0.175 mmol), CuSO₄·5H₂O (28 mg, 0.112 mmol) in water (9 mL) and THF (9 mL). Organic solvents removed under reduced pressure and aqueous solution purified by RP MPLC (H₂O with increasing MeCN (0.1% formic acid)) to give the product (275 mg, 0.385 mmol, 88%) as a white solid.

1H NMR (400 MHz, Methanol-d₄) δ 7.82 (d, J=6.6 Hz, 1H), 7.16 (d, J=8.1 Hz, 2H), 6.83 (d, J=7.8 Hz, 2H), 4.60-4.44 (m, 2H), 4.44-4.23 (m, 2H), 4.19-3.99 (m, 3H), 3.83 (dt, J=9.3, 4.6 Hz, 5H), 3.77-3.51 (m, 11H), 3.38-3.22 (m, 7H), 3.24-3.12 (m, 2H), 3.06 (dd, J=15.2, 8.5 Hz, 1H), 2.86 (q, J=7.5 Hz, 2H), 1.40 (d, J=6.4 Hz, 9H).

MS (electrospray, +ve) [M+Na]⁺ calculated for C₃₂H₅₁N₄O₁₄ requires: 715.3397, found: 715.3.

### Synthesis of I71

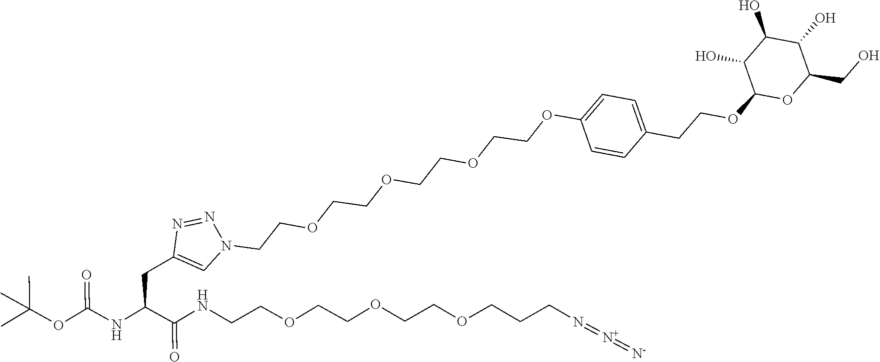 [SMILES: CC(C)(C)OC(=O)N[C@@H](Cc1cn(CCOCCOCCOCCOc2ccc(CCO[C@@H]3O[C@H](CO)[C@@H](O)[C@H](O)[C@H]3O)cc2)nn1)C(=O)NCCOCCOCCOCCCN=[N+]=[N-]]

I70 (275 mg, 0.385 mmol), EDC (111 mg, 0.577 mmol) and HOBt·H₂O (88 mg, 0.577 mmol) dissolved in DCM (22 mL) with DIPEA (0.10 mL, 0.577 mmol). After 30 min, azido-PEG₃-amine (252 mg, 1.154 mmol) added, and the reaction stirred for 18 hours. Solvents removed under reduced pressure and the crude product purified by MPLC (H₂O with increasing MeCN (0.1% formic acid)) to give the product as a clear oil (125 mg, 0.205 mmol, 46%).

1H NMR (400 MHz, Methanol-d₄) δ 8.11 (s, 1H), 7.99 (d, J=5.7 Hz, 1H), 7.82 (s, 1H), 7.16 (d, J=8.4 Hz, 2H), 6.83 (d, J=8.5 Hz, 2H), 4.50 (t, J=5.0 Hz, 2H), 4.31 (p, J=7.6 Hz, 2H), 4.13-3.96 (m, 3H), 3.84 (td, J=10.5, 9.7, 5.4 Hz, 5H), 3.77-3.56 (m, 21H), 3.51 (t, J=5.6 Hz, 2H), 3.36 (p, J=5.7 Hz, 5H), 3.29 (s, 2H), 3.22-3.09 (m, 2H), 2.86 (h, J=7.3, 6.7 Hz, 2H), 1.40 (s, 9H).

MS (electrospray, +ve) [M+H]⁺ calculated for C₄₀H₆₇N₈O₁₆ requires: 915.4670, found: 915.4.

### Synthesis of I72

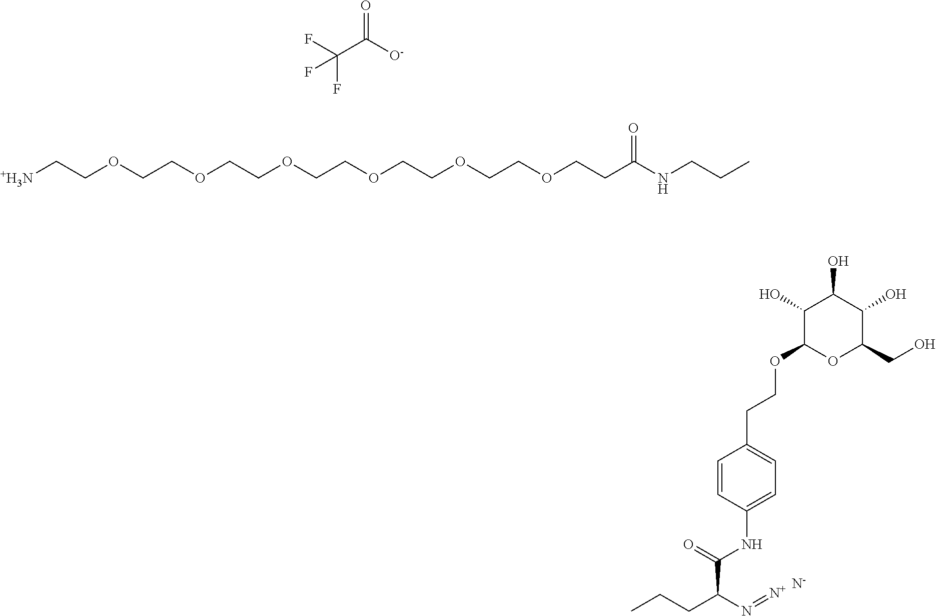

Prepared according to General Procedure D from I30 (10 mg, 0.011 mmol) in TFA (0.5 mL) and DCM (0.5 mL). Solvents blown off under a flow of N₂ and residue redissolved in DCM repeatedly (3 times) to give the product as a clear oil (9 mg, 0.011 mmol, 100%).

MS (electrospray, +ve) [M+H]⁺ calculated for C₃₅H₆₁N₆O₁₄ requires: 789.4241, found: 789.4.

### Synthesis of I73

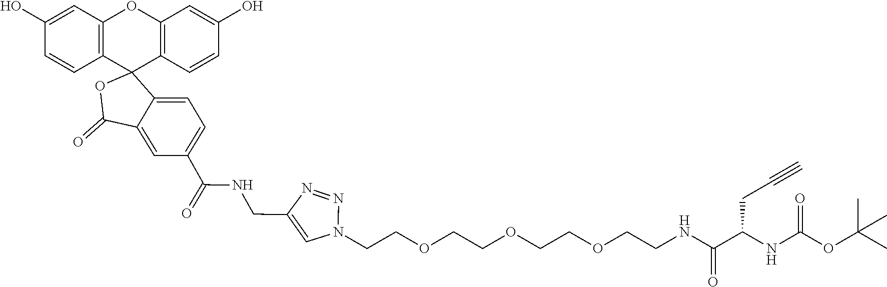 [SMILES: C#CC[C@H](NC(=O)OC(C)(C)C)C(=O)NCCOCCOCCOCCn1cc(CNC(=O)c2ccc3c(c2)C(=O)OC32c3ccc(O)cc3Oc3cc(O)ccc32)nn1]

Prepared according to General Procedure A from Boc-L-2-propargylglycine (30 mg, 0.142 mmol), I74 (30 mg, 0.047 mmol), HBTU (54 mg, 0.142 mmol), HOBt·H₂O (22 mg, 0.142 mmol) in DMF (1 mL). Product dry loaded onto C₁₈ silica and purified by RP MPLC (H₂O with increasing MeCN (0.1% formic acid)) to give the product as a yellow solid (8 mg, 0.010 mmol, 20%).

MS (electrospray, +ve) [M+H]⁺ calculated for C₄₂H₄₇N₆O₁₂ requires: 827.3246, found: 827.3.

### Synthesis of I74

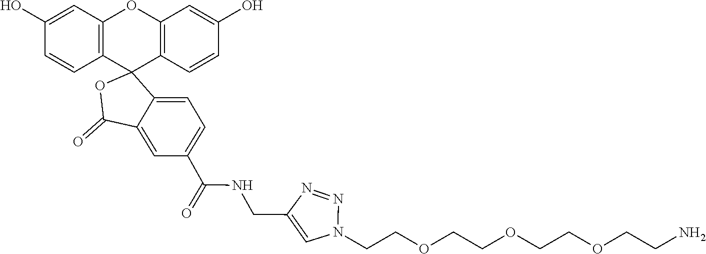 [SMILES: NCCOCCOCCOCCn1cc(CNC(=O)c2ccc3c(c2)C(=O)OC32c3ccc(O)cc3Oc3cc(O)ccc32)nn1]

Prepared according to General Procedure B from azido-PEG₃-amine (15 mg, 0.068 mmol), 5-FAM alkyne (20 mg, 0.048 mmol), sodium ascorbate (9 mg, 0.044 mmol), CuSO₄·5H₂O (12 mg, 0.048 mmol) and NaHCO₃ (6 mg, 0.068 mmol) in water (1 mL) and THF (1 mL). Organic solvents removed under reduced pressure and aqueous solution purified by RP MPLC (H₂O with increasing MeCN (0.1% formic acid)) to give the product as a yellow solid.

1H NMR (400 MHz, Methanol-d₄) δ 8.60-8.08 (m, 3H), 7.39 (s, 1H), 6.80-6.59 (m, 5H), 3.96 (s, 2H), 3.84-3.50 (m, 16H).

MS (electrospray, +ve) [M+H]⁺ calculated for C₃₂H₃₄N₅O₉ requires: 632.2352, found: 632.2.

### Synthesis of I75

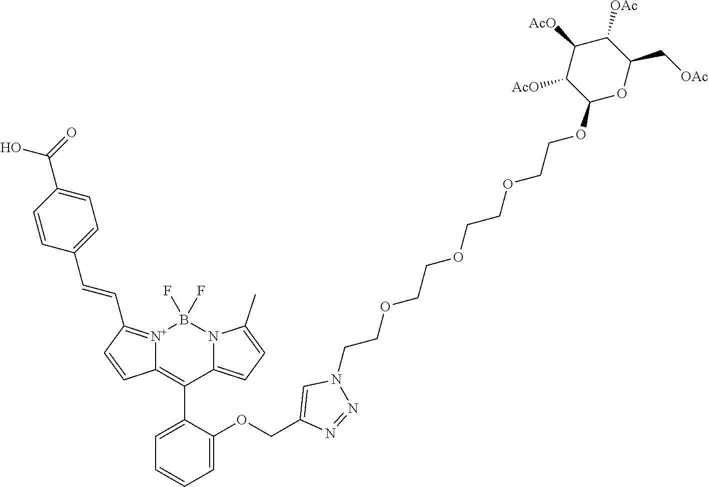

Prepared according to General Procedure B from I78 (89 mg, 0.162 mmol), I76 (30 mg, 0.062 mmol), sodium ascorbate (25 mg, 0.124 mmol), CuSO₄·5H₂O (15 mg, 0.093 mmol) in water (2 mL) and THF (6 mL). Reaction partitioned between EtOAc (30 mL) and water (30 mL). Organic layer further washed with water (30 mL) and dried (Na₂SO₄). Organic solvents removed under reduced pressure and aqueous solution purified by RP MPLC (H₂O with increasing MeCN (0.1% formic acid)) to give the product as a green solid (30 mg, 0.047 mmol, 98%).

1H NMR (400 MHz, Chloroform-d) δ 8.09 (d, J=8.0 Hz, 2H), 7.81 (d, J=16.4 Hz, 1H), 7.70 (t, J=7.7 Hz, 3H), 7.48 (d, J=8.2 Hz, 2H), 7.31 (d, J=16.6 Hz, 5H), 7.10 (t, J=7.4 Hz, 1H), 6.85 (d, J=4.4 Hz, 1H), 6.65 (dd, J=8.8, 4.3 Hz, 2H), 6.29 (d, J=4.2 Hz, 1H), 5.20 (d, J=14.5 Hz, 3H), 5.08 (t, J=9.7 Hz, 1H), 4.97 (t, J=8.9 Hz, 1H), 4.55 (d, J=8.4 Hz, 1H), 4.42 (t, J=5.4 Hz, 2H), 4.29-4.09 (m, 4H), 3.93-3.85 (m, 1H), 3.77 (t, J=5.5 Hz, 2H), 3.67 (s, 3H), 3.58-3.40 (m, 11H), 2.70 (s, 3H), 2.08 (s, 3H), 2.04-1.97 (m, 9H).

MS (electrospray, +ve) [M+H]⁺ calculated for C₅₀H₅₇BF₂N₅O₁₆ requires: 1032.3856, found: 1032.3.

### Synthesis of I76

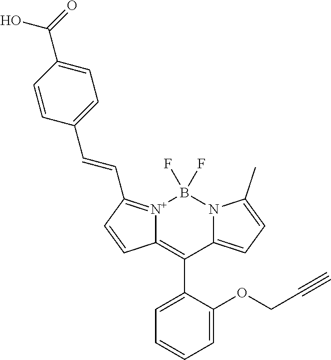

I77 100 mg, 0.286 mmol), 4-formylbenzoic acid (43 mg, 0.286 mmol) and ammonium acetate (66 mg, 0.857 mmol) dissolved in DMF (7 mL) and heated at 140° C. for 30 minutes. Solvents removed under reduced pressure and the residue dry loaded onto silica and purified by MPLC (DCM with increasing MeOH (10% AcOH)) followed by RP MPLC (H₂O with increasing MeCN (0.1% formic acid)) to give the product (29 mg, 0.060 mmol, 21%).

1H NMR (400 MHz, Chloroform-d) δ 8.10 (d, J=8.4 Hz, 2H), 7.82 (d, J=16.3 Hz, 1H), 7.68 (d, J=8.4 Hz, 2H), 7.48 (ddd, J=8.4, 7.4, 1.8 Hz, 1H), 7.34-7.26 (m, 2H), 7.19 (dd, J=8.5, 1.0 Hz, 1H), 7.10 (td, J=7.5, 1.0 Hz, 1H), 6.86 (d, J=4.4 Hz, 1H), 6.69-6.66 (m, 2H), 6.28 (d, J=4.2 Hz, 1H), 4.65 (d, J=2.4 Hz, 2H), 2.70 (s, 3H), 2.46 (t, J=2.4 Hz, 1H).

### Synthesis of I77

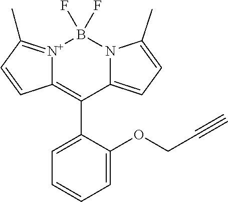

2-(2-Propynyloxy)benzenecarbaldehyde (1.750 g, 0.011 mol) dissolved in degassed DCM (65 mL) in darkness, 2-methyl-1H-pyrrole (1.950 mg, 0.024 mol) added and solution degassed again. TFA (0.05 mL, 0.001 mol) added, and reaction stirred for 90 minutes. DDQ (2.480 g, 0.011 mol) added in five portions over 30 minutes then reaction stirred for a further 90 minutes. Et₃N (6.4 mL, 0.046 mol) added dropwise and after 5 minutes BF₃·OEt₂ (8.6 mL, 0.069 mol) added. After 90 min, the reaction was dry loaded onto silica and passed through a silica plug (eluting DCM:Petrol, 4:1) and the coloured organics were diluted with EtOAc (100 mL) and washed with 1 M HCl (5×50 mL), 2 M NaOH (5×50 mL), 1 M HCl (50 mL), 2 M NaOH (50 mL) and brine (50 mL). Organic layer dried (Na₂SO₄) and concentrated to give the product (1.80 g, 0.005 mol, 47%).

1H NMR (400 MHz, Chloroform-d) δ 7.46 (dddd, J=8.3, 7.3, 1.8, 0.9 Hz, 1H), 7.30-7.25 (m, 1H), 7.18 (dd, J=8.5, 1.0 Hz, 1H), 7.08 (td, J=7.5, 1.0 Hz, 1H), 6.60 (d, J=4.1 Hz, 2H), 6.20 (d, J=4.1 Hz, 2H), 4.63 (dd, J=2.4, 0.9 Hz, 2H), 2.64 (d, J=1.3 Hz, 6H), 2.45 (td, J=2.3, 0.9 Hz, 1H).

### Synthesis of I78

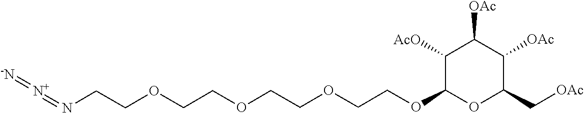

2-[2-[2-(2-azidoethoxy)ethoxy]ethoxy]ethanol (5.915 g, 25.6 mol) dissolved in DCM (120 mL) and acetobromo-α-D-glucose (13.501 g, 32.833 mmol) and silver carbonate (9.202 g, 33.038 mmol) added and the reaction stirred in darkness for 18 hours. The reaction mixture was filtered through celite and concentrated under reduced pressure. Acetic acid (30 mL) and water (1.5 mL) were added, and the reaction stirred for 20 minutes. Solvents removed under a stream of N₂ and resultant residue stirred in petrol for 18 hours. Supernatant petrol decanted and pyridine (10 mL) and Ac₂O (5 mL, 53.005 mmol) added, and reaction stirred for 2 hours. Solvents removed under a stream of N₂ and the resultant oil dissolved in DCM (100 mL) and washed with 1 M HCl (50 mL). Organics dried (Na₂SO₄) and evaporated to a gum. Purified by MPLC (petrol with increasing Et₂O) to give the product as a colourless oil.

1H NMR (400 MHz, Methanol-d₄) δ 5.25 (t, J=9.5 Hz, 1H), 5.02 (dd, J=10.1, 9.4 Hz, 1H), 4.91-4.85 (m, 1H), 4.75 (d, J=8.0 Hz, 1H), 4.28 (dd, J=12.3, 4.6 Hz, 1H), 4.14 (dd, J=12.3, 2.5 Hz, 1H), 3.95-3.84 (m, 2H), 3.75 (ddd, J=11.3, 6.1, 3.9 Hz, 1H), 3.70-3.61 (m, 14H), 3.42-3.35 (m, 2H), 2.06 (s, 3H), 2.03 (s, 3H), 2.01 (s, 3H), 1.97 (s, 3H).

### Synthesis of I79

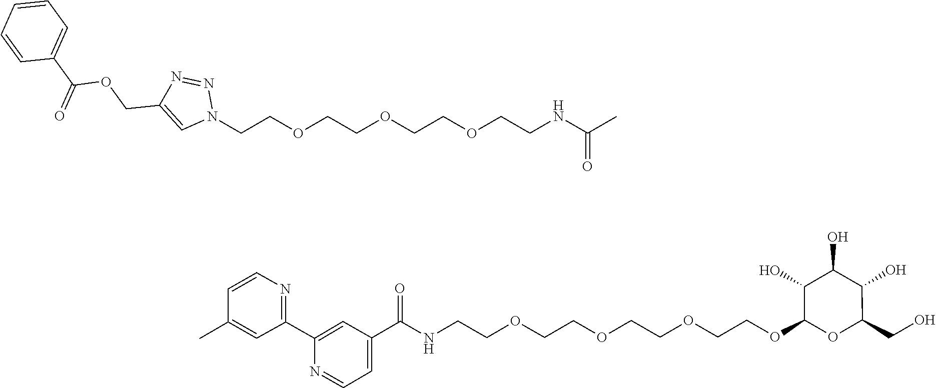

Prepared according to General Procedure B from I80 (77 mg, 0.098 mmol), prop-2-yn-1-yl benzoate (15 mg, 0.094 mmol), sodium ascorbate (37 mg, 0.187 mmol), CuSO₄·5H₂O (22 mg, 0.140 mmol) in water (1 mL) and THF (3 mL). Organic solvents removed under reduced pressure and aqueous solution purified by RP MPLC (H₂O with increasing acetone) to give the product (35 mg, 0.037 mmol, 40%).

1H NMR (400 MHz, Methanol-d₄) δ 8.79 (s, 3H), 8.13 (s, 1H), 8.00 (dd, J=15.5, 7.5 Hz, 2H), 7.79 (s, 2H), 7.58 (t, J=7.4 Hz, 1H), 7.44 (t, J=7.6 Hz, 2H), 5.41 (s, 2H), 4.27 (d, J=7.6 Hz, 1H), 3.74-3.52 (m, 30H), 3.26 (d, J=7.1 Hz, 2H), 3.17 (t, J=8.3 Hz, 2H).

MS (electrospray, +ve) [M+H]⁺ calculated for C₄₄H₆₀N₇O₁₆ requires: 942.4092, found: 942.4.

### Synthesis of I80

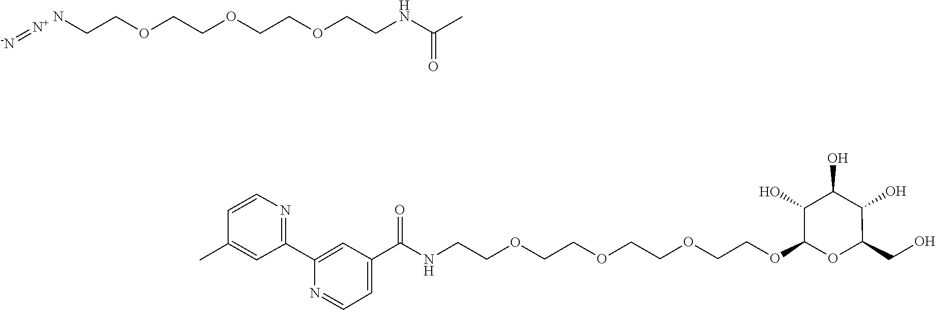

Prepared according to General Procedure A from I81 (200 mg, 0.450 mmol), I82 (192 mg, 0.540 mmol), HBTU (256 mg, 0.675 mmol), HOBt·H₂O (103 mg, 0.675 mmol) and DIPEA (0.12 mL, 0.675 mmol) in DMF (4.5 mL). Organic solvents removed under reduced pressure and residue purified by RP MPLC (H₂O with increasing acetone) to give the product (119 mg, 0.152 mmol, 34%).

1H NMR (400 MHz, Methanol-d₄) δ 8.84 (dt, J=5.0, 0.9 Hz, 2H), 8.79 (dt, J=1.8, 0.9 Hz, 2H), 7.81 (dt, J=5.0, 1.9 Hz, 2H), 4.27 (d, J=7.8 Hz, 1H), 3.96 (ddd, J=10.6, 5.1, 3.4 Hz, 1H), 3.88-3.81 (m, 1H), 3.73-3.58 (m, 31H), 3.37-3.32 (m, 2H), 3.26-3.23 (m, 1H), 3.18 (dd, J=9.1, 7.8 Hz, 1H).

MS (electrospray, +ve) [M+H]⁺ calculated for C₃₄H₅₂N₇O₁₄ requires: 782.82454, found: 782.8.

### Synthesis of I81

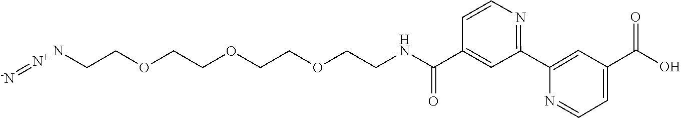

Prepared according to General Procedure A from [2,2′-bipyridine]-4,4′-dicarboxylic acid (1.00 g, 4.095 mmol), azido-PEG₃-amine (894 mg, 4.095 mmol), HBTU (3.11 g, 8.19 mmol), and DIPEA (2.9 mL, 16.38 mmol) in DMF (200 mL). Majority of DMF removed under reduced pressure and remnants diluted in EtOAc and washed with 5% LiCl (aq.). Organics dried (Na₂SO₄) and concentrated under reduced pressure. Residue purified by RP MPLC (H₂O with increasing MeCN (0.1% formic acid)) to give the product (390 mg, 0.877 mmol, 21%).

1H NMR (400 MHz, Methanol-d₄) δ 8.89-8.70 (m, 4H), 7.91 (td, J=4.0, 3.1, 1.6 Hz, 1H), 7.77 (dt, J=4.0, 2.0 Hz, 1H), 3.71-3.53 (m, 14H), 3.27 (d, J=4.3 Hz, 2H).

MS (electrospray, +ve) [M+H]⁺ calculated for C₃₄H₅₂N₇O₁₄ requires: 782.82454, found: 782.8.

### Synthesis of I82

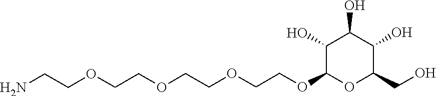 [SMILES: NCCOCCOCCOCCO[C@@H]1O[C@H](CO)[C@@H](O)[C@H](O)[C@H]1O]

To a solution of I83 (107 mg, 0.281 mmol) in H₂O (5.5 mL) and THF (5.5 mL) was added Pd/C (45 mg, 10% w/w). Reaction stirred overnight under an atmosphere of hydrogen at RT. Filtered through celite with THF/H₂O and concentrated. Product obtained as a white solid (83 mg, 0.234 mmol, 83%).

1H NMR (400 MHz, Methanol-d₄) δ 4.31 (dd, J=7.8, 1.8 Hz, 1H), 4.02 (ddd, J=10.8, 5.1, 3.2 Hz, 1H), 3.86 (dd, J=11.9, 1.7 Hz, 1H), 3.77-3.58 (m, 14H), 3.53 (t, J=5.3 Hz, 1H), 3.28 (dt, J=5.2, 1.9 Hz, 2H), 3.24-3.16 (m, 1H), 2.79 (dd, J=6.1, 4.5 Hz, 2H).

### Synthesis of I83

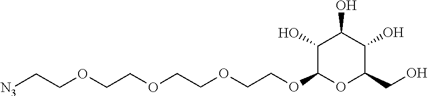 [SMILES: [N-]=[N+]=NCCOCCOCCOCCO[C@@H]1O[C@H](CO)[C@@H](O)[C@H](O)[C@H]1O]

To a solution of I78 (6.00 g, 10.919 mmol) in MeOH (16 mL) was added Na₂CO₃ (1.74 g, 16.378 mmol) and reaction stirred overnight. Supernatant MeOH was adsorbed onto silica and the purified by MPLC (EtOAc with increasing MeOH) to give the product (3.218 g, 8.44 mmol, 77%)

1 H NMR (400 MHz, Deuterium Oxide) δ 4.51 (dd, J=8.0, 1.0 Hz, 1H), 4.14-4.04 (m, 1H), 4.00-3.84 (m, 1H), 3.84-3.65 (m, 14H), 3.60-3.45 (m, 3H), 3.45-3.40 (m, 1H), 3.40-3.36 (m, 1H), 3.32 (ddd, J=9.2, 8.0, 1.0 Hz, 1H).

### Synthesis of I84

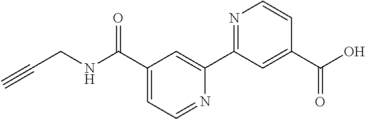 [SMILES: C#CCNC(=O)c1ccnc(-c2cc(C(=O)O)ccn2)c1]

Prepared according to General Procedure A from [2,2′-bipyridine]-4,4′-dicarboxylic acid (500 mg, 2.05 mmol), propargylamine (0.085 mL, 1.331 mmol), HBTU (1.55 g, 4.10 mmol), and DIPEA (1.4 mL, 8.19 mmol) in DMF (18 mL) at 45° C. Crude product purified by RP MPLC (H₂O with increasing MeCN (0.1% formic acid)) to give the product as a brown solid (70 mg, 0.220 mmol, 11%).

¹H NMR (400 MHz, DMSO-d₆) δ 9.45 (t, J=5.6 Hz, 1H), 8.91 (d, J=5.0 Hz, 1H), 8.87 (d, J=5.0 Hz, 1H), 8.84 (s, 1H), 8.80 (s, 1H), 7.96-7.88 (m, 1H), 7.85 (d, J=5.0 Hz, 1H), 4.10 (dd, J=5.6, 2.5 Hz, 2H), 1.25 (d, J=6.6 Hz, 1H).

MS (electrospray, +ve) [M+H]⁺ calculated for C₁₅H₁₂N₃O₃ requires: 282.0874, found: 282.1.

### Synthesis of I85

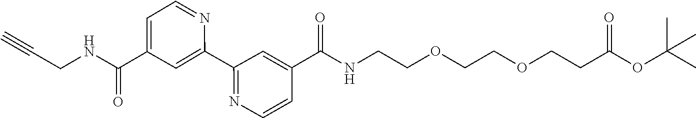 [SMILES: C#CCNC(=O)c1ccnc(-c2cc(C(=O)NCCOCCOCCC(=O)OC(C)(C)C)ccn2)c1]

Prepared according to General Procedure A from I84 (30 mg, 0.107 mmol), tert-butyl 3-[2-(2-aminoethoxy)ethoxy]propanoate (37 mg, 0.160 mmol), HBTU (81 mg, 0.213 mmol), and DIPEA (0.074 mL, 0.427 mmol) in DMF (1 mL). Crude product purified by RP MPLC (H₂O with increasing MeCN (0.1% formic acid)) to give the product as a clear oil (18 mg, 0.036 mmol, 34%).

¹H NMR (400 MHz, Methanol-d₄) δ 8.83 (dd, J=5.1, 3.1 Hz, 2H), 8.79 (s, 2H), 7.86-7.76 (m, 2H), 4.21 (d, J=2.5 Hz, 2H), 3.79-3.58 (m, 10H), 2.66 (t, J=2.6 Hz, 1H), 2.46 (t, J=6.2 Hz, 2H), 1.40 (s, 9H).

MS (electrospray, +ve) [M+H]⁺ calculated for C₂₆H₃₃N₄O₆ requires: 497.2395, found: 497.2.

### Synthesis of I86

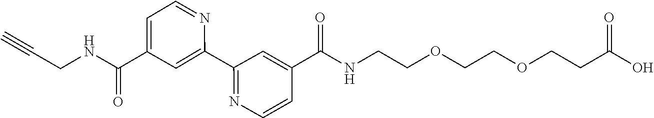 [SMILES: C#CCNC(=O)c1ccnc(-c2cc(C(=O)NCCOCCOCCC(=O)O)ccn2)c1]

Prepared according to General Procedure D from I85 (18 mg, 0.036 mmol) in TFA (0.5 mL) and DCM (1 mL). Product obtained as a glassy solid (11 mg, 0.014 mmol, 95%).

1H NMR (400 MHz, Methanol-d₄) δ 8.83 (dd, J=5.1, 3.2 Hz, 2H), 8.78 (s, 2H), 7.80 (td, J=5.2, 1.7 Hz, 2H), 4.21 (d, J=2.6 Hz, 2H), 3.75 (t, J=6.2 Hz, 2H), 3.72-3.57 (m, 8H), 2.66 (t, J=2.6 Hz, 1H), 2.53 (t, J=6.2 Hz, 2H).

### Synthesis of I87

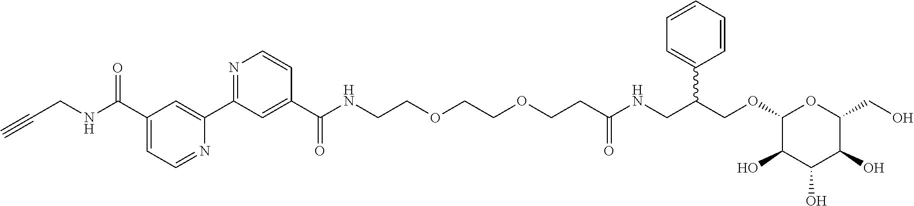 [SMILES: C#CCNC(=O)c1ccnc(-c2cc(C(=O)NCCOCCOCCC(=O)NCC(CO[C@@H]3O[C@H](CO)[C@@H](O)[C@H](O)[C@H]3O)c3ccccc3)ccn2)c1]

Prepared according to General Procedure A from I86 (10 mg, 22.7 μmol), I94 (12 mg, 34.1 μmol), HBTU (10 mg, 25.0 μmol), HOBt·H₂O (45 mg, 45.4 μmol) and DIPEA (0.016 mL, 90.8 μmol) in DMF (1 mL). Crude product purified by RP MPLC (H₂O with increasing MeCN (0.1% formic acid)) to give the product as a white solid (12 mg, 12.2 μmol, 54%).

1H NMR (400 MHz, Methanol-d₄) δ 8.82 (dd, J=5.1, 2.2 Hz, 2H), 8.78 (d, J=1.7 Hz, 2H), 7.79 (ddd, J=5.1, 3.5, 1.7 Hz, 2H), 7.28-7.14 (m, 5H), 4.28 (dd, J=7.8, 4.0 Hz, 1H), 4.21 (d, J=2.5 Hz, 2H), 4.10-4.02 (m, 1H), 3.87 (dt, J=11.7, 2.2 Hz, 1H), 3.78-3.53 (m, 13H), 3.51-3.44 (m, 1H), 3.27 (dq, J=6.3, 1.6 Hz, 2H), 3.25-3.16 (m, 1H), 3.13 (dq, J=7.5, 4.7, 4.2 Hz, 1H), 2.66 (t, J=2.5 Hz, 1H), 2.36 (td, J=6.1, 3.4 Hz, 2H).

MS (electrospray, +ve) [M+H]⁺ calculated for C₃₇H₄₆N₅O₁₁ requires: 736.3189, found: 736.3.

### Synthesis of I88

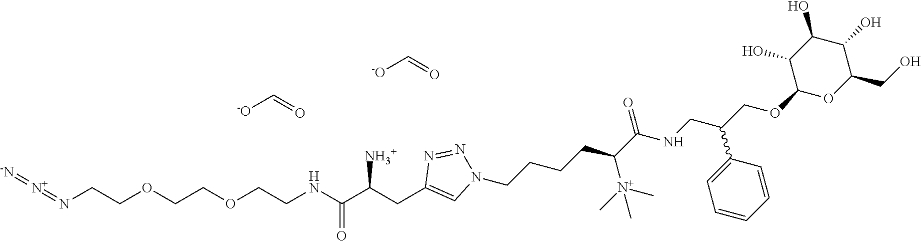

Prepared according to General Procedure D from I89 (20 mg, 0.022 mmol) in TFA (0.3 mL) and DCM (0.3 mL). Solvents removed and residue purified by RP MPLC (H₂O with increasing MeCN (0.1% formic acid)) to give the product obtained as a clear solid (13 mg, 0.016 mmol, 73%).

MS (electrospray, +ve), [M]⁺ calculated for C₃₅H₅₉N₁₀O₁₀ requires: 779.4410, found: 779.4.

### Synthesis of I89

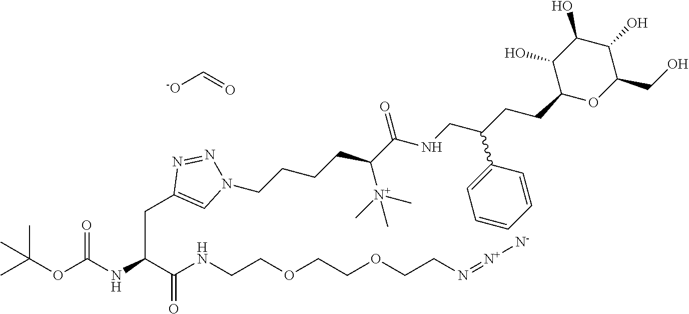

Prepared according to General Procedure A from I90 (136 mg, 0.188 mmol), 2-(2-(2-azidoethoxy)ethoxy)ethan-1-amine (60 mg, 0.344 mmol), HATU (122 mg, 0.376 mmol), and DIPEA (0.10 mL, 0.564 mmol) in DMF (1 mL). Crude product purified by RP HPLC (H₂O with increasing MeCN (0.1% formic acid)) to give the product as a white solid (80 mg, 0.086 mmol, 46%).

MS (electrospray, +ve) [M]⁺ calculated for C₄₀H₆₇N₁₀O₁₂ requires: 879.4935, found: 879.4.

### Synthesis of I90

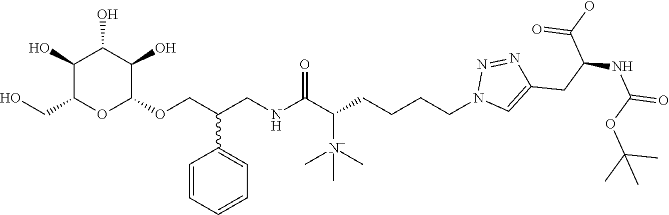

Prepared according to General Procedure B from I91 (137 mg, 0.247 mmol), Boc-L-2-propargylglycine (61 mg, 0.284 mmol), sodium ascorbate (49 mg, 0.247 mmol), CuSO₄·5H₂O (19 mg, 0.074 mmol) in water (1 mL) and THF (1 mL). Purified by RP MPLC (H₂O with increasing MeCN (0.1% formic acid)) to give the product as a white solid (136 mg, 0.188 mmol, 76%).

MS (electrospray, +ve), [M+H]⁺ calculated for C₃₄H₅₅N₆O₁₁ requires: 723.3924, found: 723.4.

### Synthesis of I91

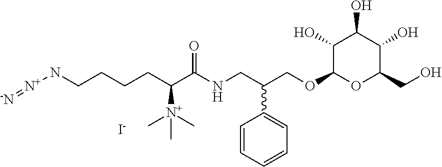

To a solution of I92 (115 mg, 0.246 mmol) in MeOH (1 mL) was DIPEA (0.21 mL, 1.23 mmol) and methyl iodide (0.77 mL, 12.3 mmol). Reaction stirred for 1.5 hours at RT before solvents were removed under vacuum. Purified by RP MPLC (H₂O with increasing MeCN (0.1% formic acid)). Product obtained as a white solid (145 mg, 0.227 mmol, 92%).

MS (electrospray, +ve) [M]⁺ calculated for C₂₄H₄₀N₅O₇ requires: 510.2923, found: 510.3.

### Synthesis of I92

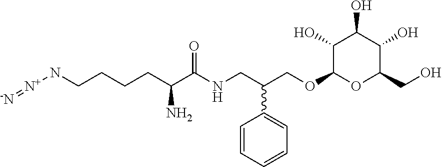

I93 (207 mg, 0.300 mmol) was dissolved in DMF (4 mL), piperidine (0.11 mL, 1.13 mmol) added and the reaction stirred at RT for 1.5 hours. The solvent removed under a flow of nitrogen and partitioned between dichloromethane/methanol (9:1) and water (neutralised to pH 7). The aqueous layer was collected and purified by RP MPLC (water with increasing acetonitrile (0.1% formic acid)). The solvent was removed under vacuum to give the product (147 mg, 0.195 mmol, 98%) as a colourless gummy solid.

MS (electrospray, +ve) [M+H]⁺ calculated for C₂₁H₃₄N₅O₇ requires: 468.2453, found: 468.2.

### Synthesis of I93

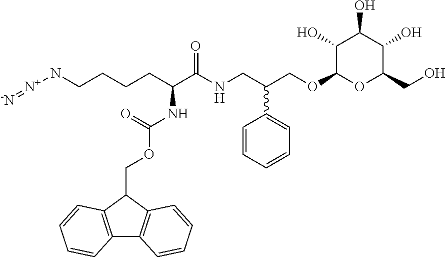

Prepared according to General Procedure A from Fmoc-azido-L-lysine (145 mg, 0.368 mmol), I94 (112 mg, 0.320 mmol), HATU (156 mg, 0.480 mmol), and DIPEA (0.08 mL, 0.480 mmol) in DMF (1 mL). Crude product purified by RP MPLC (H₂O with increasing MeCN (0.1% formic acid)) to give the product as a brown solid (70 mg, 0.220 mmol, 11%).

MS (electrospray, +ve) [M+H]⁺ calculated for C₃₆H₄₄N₅O₉ requires: 690.3134, found: 690.3.

### Synthesis of I94

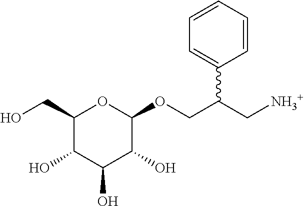 [SMILES: [NH3+]CC(CO[C@@H]1O[C@H](CO)[C@@H](O)[C@H](O)[C@H]1O)c1ccccc1]

I95 (480 mg, 1.414 mmol) dissolved in degassed water (25 mL) under an atmosphere of N₂. PPh₃ (445 mg, 1.697 mmol) was added, followed by THF (50 mL). The reaction was stirred for 21 hours. Solvent was removed under reduced pressure and the residue partitioned between dilute HCl (pH 6, 50 mL) and DCM (50 mL). The aqueous was further washed with DCM (2×50 mL) then slightly concentrated under reduced pressure. Lyophilisation gave the product as a clear solid (490 mg, 1.401 mmol, 99%).

¹H NMR (400 MHz, Deuterium Oxide) δ 7.36-7.30 (m, 2H), 7.27 (tt, J=7.9, 1.3 Hz, 3H), 4.35 (dd, J=13.9, 7.9 Hz, 1H), 4.09-4.01 (m, 1H), 3.85-3.74 (m, 2H), 3.58 (ddd, J=12.3, 5.8, 4.2 Hz, 1H), 3.46-3.18 (m, 6H), 3.15 (ddd, J=9.3, 8.0, 3.7 Hz, 1H).

### Synthesis of I95

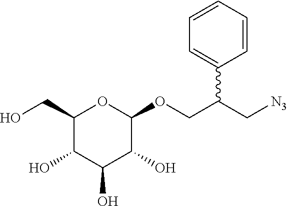 [SMILES: [N-]=[N+]=NCC(CO[C@@H]1O[C@H](CO)[C@@H](O)[C@H](O)[C@H]1O)c1ccccc1]

To a stirred suspension of I96 (279 mg, 0.550 mmol) in MeOH (10 mL) was added sodium methoxide (6 mg, 0.110 mmol). After 30 minutes the reaction mixture was passed through a bed of regenerated H+ Dowex (10 g) washing through with MeOH.

The solvent was removed which gave a white foam (180 mg, 0.530 mmol, 97%).

¹H NMR (400 MHz, Methanol-d₄) δ 7.35-7.28 (m, 4H), 7.28-7.21 (m, 1H), 4.28 (dd, J=7.8, 1.1 Hz, 1H), 4.15-4.07 (m, 1H), 3.89-3.84 (m, 1H), 3.83-3.73 (m, 2H), 3.69-3.59 (m, 2H), 3.37-3.32 (m, 1H), 3.29-3.24 (m, 2H), 3.24-3.16 (m, 2H).

MS (electrospray, +ve) [M+H]⁺ calculated for C₁₅H₂₁N₃NaO₆ requires: 362.1323, found: 362.1.

### Synthesis of I96

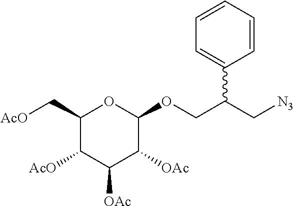 [SMILES: CC(=O)OC[C@H]1O[C@@H](OCC(CN=[N+]=[N-])c2ccccc2)[C@H](OC(C)=O)[C@@H](OC(C)=O)[C@@H]1OC(C)=O]

I97 (1.65 g, 2.94 mmol) dissolved in DMF (12 mL), NaN₃ (574 mg, 8.83 mmol) added, and the reaction stirred at 80° C. for 1.5 hours. The reaction mixture was diluted with water (200 mL) and extracted with EtOAc (100 mL). The organic layer was further washed with water (2×200 mL) and brine (100 mL), dried (MgSO₄) and concentrated under reduced pressure. Crude oil was purified by RP MPLC (water with increasing MeOH) to give the product an amorphous solid (1.16 g, 2.30 mmol, 78%).

¹H NMR (400 MHz, Chloroform-d) δ 7.36-7.26 (m, 3H), 7.25-7.19 (m, 2H), 5.19 (td, J=9.5, 7.8 Hz, 1H), 5.08 (ddd, J=10.0, 9.4, 4.1 Hz, 1H), 5.01 (ddd, J=16.8, 9.6, 7.9 Hz, 1H), 4.49 (dd, J=11.6, 7.9 Hz, 1H), 4.25 (dd, J=12.3, 4.7 Hz, 1H), 4.17-4.10 (m, 2H), 3.75-3.60 (m, 3H), 3.60-3.50 (m, 1H), 3.17-3.06 (m, 1H), 2.08 (s, 3H), 2.02 (s, 1.5H), 2.02 (s, 1.5H), 2.01 (s, 1.5H), 2.00 (s, 1.5H), 1.99 (s, 1.5H), 1.89 (s, 1.5H).

### Synthesis of I97

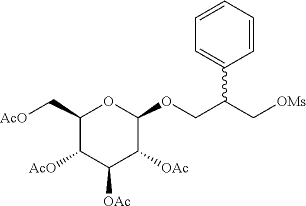 [SMILES: CC(=O)OC[C@H]1O[C@@H](OCC(COS(C)(=O)=O)c2ccccc2)[C@H](OC(C)=O)[C@@H](OC(C)=O)[C@@H]1OC(C)=O]

I98 (1.50 g, 3.11 mmol) dissolved in THF (15 mL) and cooled to 0° C., Et₃N (0.69 mL, 4.97 mmol) added, followed by addition of MsCl (0.62 mL, 4.97 mmol) over a period of 10 min. After 5 more minutes reaction diluted with 0.2 M HCl (100 mL) and partitioned with EtOAc (50 mL) The organic was washed with water (2×100 mL) and brine (60 mL), dried (Na₂SO₄) and concentrated under reduced pressure to give the crude product as a white foam (1.65 g, 2.94 mmol, 95%).

¹H NMR (400 MHz, Chloroform-d) δ 7.35-7.27 (m, 3H), 7.25-7.19 (m, 2H), 5.21-5.15 (m, 1H), 5.12-4.95 (m, 2H), 4.51 (dd, J=7.9, 6.0 Hz, 1H), 4.48-4.35 (m, 2H), 4.32-4.16 (m, 2H), 4.17-4.10 (m, 1H), 3.76 (ddd, J=9.6, 7.6, 6.6 Hz, 1H), 3.72-3.64 (m, 1H), 3.30 (dt, J=10.7, 6.1 Hz, 1H), 2.88 (s, 1.5H), 2.87 (s, 1.5H), 2.08 (s, 1.5H), 2.08 (s, 1.5H), 2.02 (s, 1.5H), 2.02 (s, 1.5H), 2.00 (s, 1.5H), 2.00 (s, 1.5H), 1.99 (s, 1.5H), 1.92 (s, 1.5H).

### Synthesis of I98

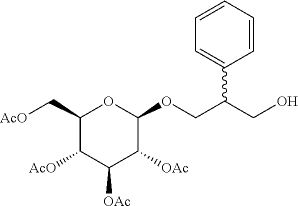 [SMILES: CC(=O)OC[C@H]1O[C@@H](OCC(CO)c2ccccc2)[C@H](OC(C)=O)[C@@H](OC(C)=O)[C@@H]1OC(C)=O]

To a suspension of silver carbonate (5.44 g, 19.7 mmol) and 2-phenylpropane-1,3-diol (1.50 g, 9.86 mmol) in DCM (20 mL) was added iodine (27 mg, 0.106 mmol). After stirring at RT for 10 min, acetobromo-α-D-glucose (4.40 g, 10.7 mmol) was added. After 1.5 h, reaction mixture was filtered through a bed of celite before solvent was removed under vacuum and purified by MPLC (petrol with increasing EtOAc). Product obtained as a colourless solid (4.42 g, 9.16 mmol, 93%).

¹H NMR (400 MHz, Chloroform-d) δ 7.35-7.27 (m, 2H), 7.25-7.20 (m, 3H), 5.29-5.13 (m, 2H), 5.13-4.95 (m, 2H), 4.88 (dd, J=9.8, 8.1 Hz, 0.5H), 4.73 (dd, J=8.8, 8.1 Hz, 0.5H), 4.53 (dd, J=13.2, 8.0 Hz, 1H), 4.29-4.12 (m, 3H), 3.99-3.63 (m, 3H), 3.16-3.05 (m, 1H), 2.10-2.09 (m, 4.5H), 2.08 (s, 1.5H), 2.03-2.01 (m, 3H), 2.01-2.00 (m, 3H).

### Synthesis of I99

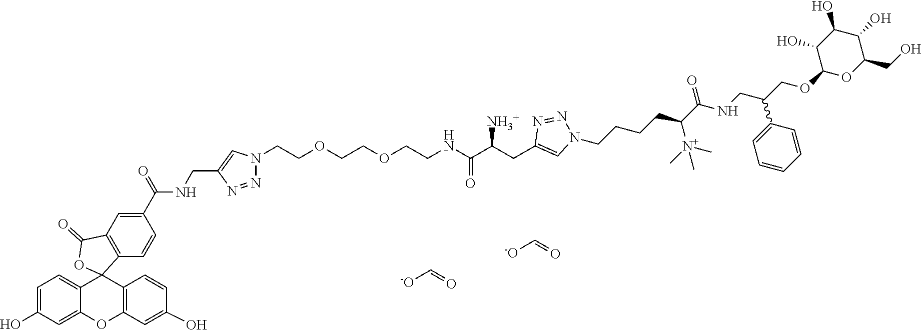

Prepared according to General Procedure D from I100 (5 mg, 3.7 μmol) in TFA (0.8 mL) and DCM (1 mL). Purified by RP MPLC (H₂O with increasing MeCN (0.1% formic acid)) to give the product as an orange solid (3.5 mg, 2.7 μmol, 73%).

MS (electrospray, +ve), [M]⁺ calculated for C₅₉H₇₄N₁₁O₁₆ requires: 1192.5310, found: 1192.4.

### Synthesis of I100

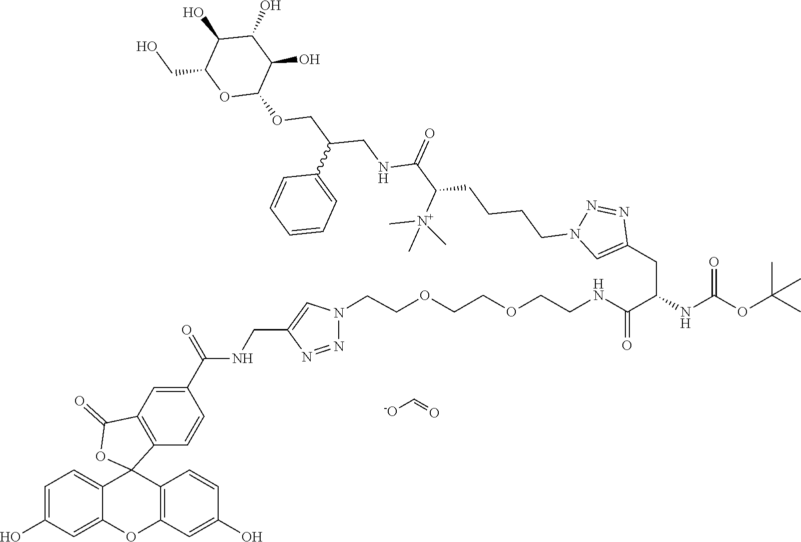

Prepared according to General Procedure B from 5-FAM alkyne (4 mg, 10 μmol), I89 (5 mg, 5.4 μmol), sodium ascorbate (3 mg, 16 μmol), CuSO₄·5H₂O (1 mg, 4.2 μmol) in water (0.3 mL) and THF (0.3 mL). Purified by RP MPLC (H₂O with increasing MeCN (0.1% formic acid)) to give the product as an orange solid (2.8 mg, 2.1 μmol, 39%).

MS (electrospray, +ve), [M]⁺ calculated for C₆₄H₈₂N₁₁O₁₈ requires: 1292.5834, found: 1292.5.

### Synthesis of I101

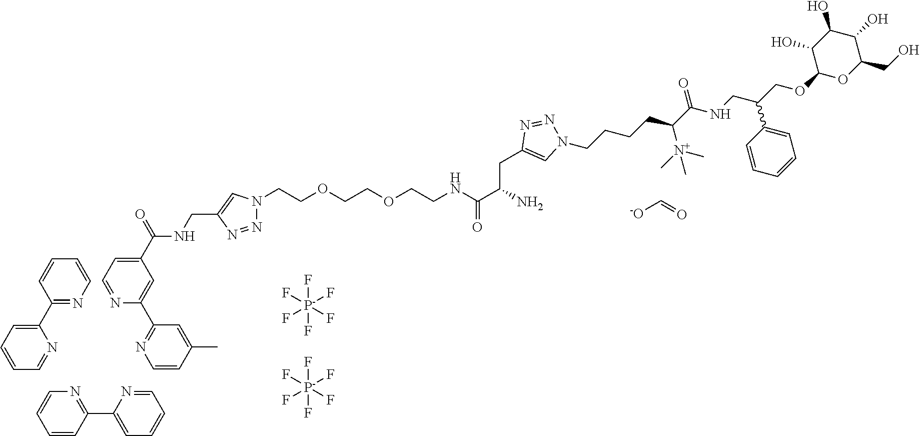

Prepared according to General Procedure B from Ru(bpy)3 alkyne (12 mg, 12.2 μmol), I88 (12 mg, 13.8 μmol), sodium ascorbate (5 mg, 26 μmol), CuSO₄·5H₂O (3 mg, 12 μmol) and NaHCO₃ (5 mg, 25.2 μmol) in water (1 mL). Purified by RP MPLC (H₂O with increasing MeCN (0.1% formic acid)) to give the product as an orange solid (9 mg, 5.8 μmol, 48%).

MS (electrospray, +ve), [M]³⁺ calculated for C₇₀H₈₈N₁₇O₁₁Ru requires: 481.5292, found: 481.5.

### Synthesis of I102

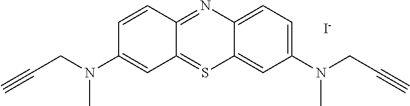

Phenothiazine-5-ium tetraiodide hydrate (200 mg, 0.277 mmol) dissolved in MeOH. N-methylprop-2-yn-1-amine (295 mg, 4.27 mmol) was added, and the reaction stirred for 45 minutes then filtered. The solids were washed with MeOH (50 mL) and the combined filtrate concentrated under reduced vacuum. The residue was redissolved in hot MeOH (6 mL) and precipitated with Et₂O (20 mL). The supernatant was decanted, and the remnants redissolved in hot MeOH and reprecipitated with Et₂O. After 16 hours at 5° C. the precipitate was collected by centrifugation to give the product as a black solid (50 mg, 0.108 mmol, 39%).

¹H NMR (400 MHz, DMSO-d₆) δ 8.16-7.79 (m, 2H), 7.58-7.42 (m, 2H), 7.39-7.17 (m, 2H), 4.70-4.31 (m, 4H), 3.7-3.29 (m, 6H), 3.16-3.07 (m, 2H).

### Synthesis of I103

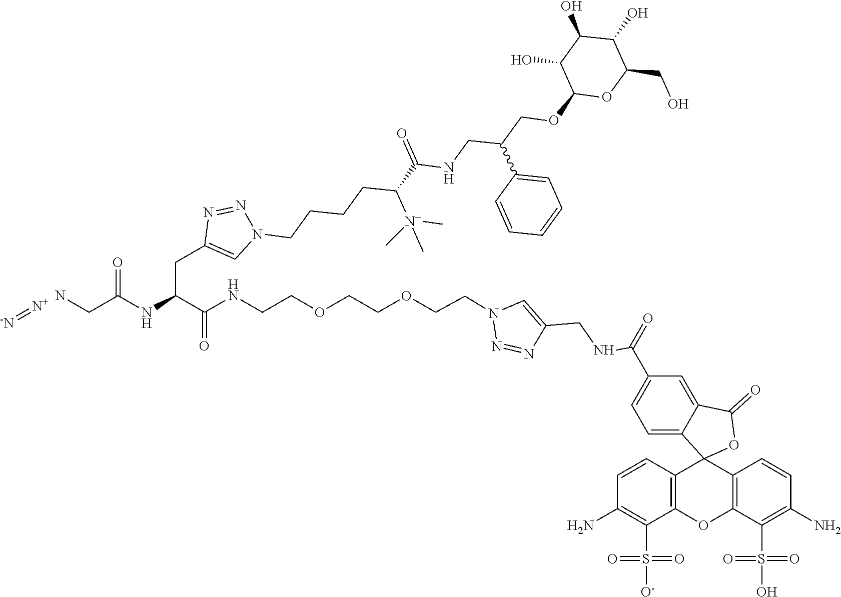

Prepared according to General Procedure C from I104 (1.5 mg, 0.95 μmol), azidoacetic acid NHS ester (1 mg, 5.0 μmol) and DIPEA (1 μL, 5.7 μmol) in DMF (0.5 mL). Purified by RP HPLC (H₂O with increasing MeCN (0.1% formic acid)). Product obtained as a red solid (1.4 mg, 0.92 μmol, 97%).

MS (electrospray, +ve), [M+H]⁺ calculated for C₆₁H₇₇N₁₆O₂₁S₂ requires: 1433.4886, found: 1433.4.

### Synthesis of I104

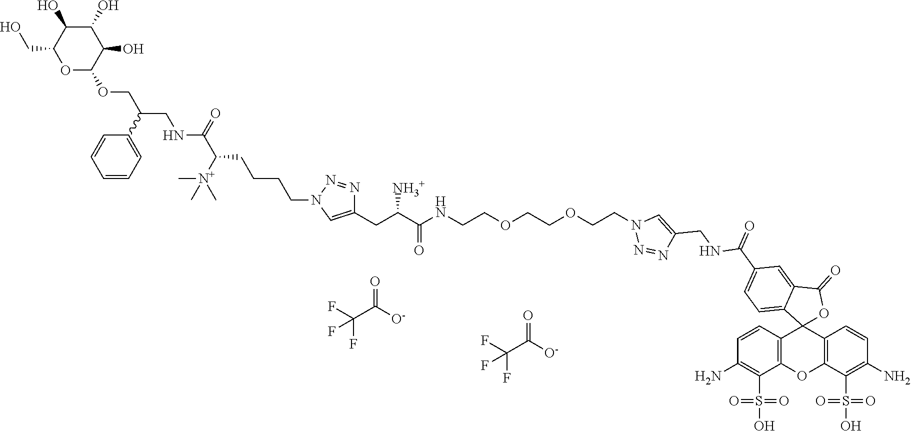 [SMILES: C[N+](C)(C)[C@@H](CCCCn1cc(C[C@H]([NH3+])C(=O)NCCOCCOCCn2cc(CNC(=O)c3ccc4c(c3)C(=O)OC43c4ccc(N)c(S(=O)(=O)O)c4Oc4c3ccc(N)c4S(=O)(=O)O)nn2)nn1)C(=O)NCC(CO[C@@H]1O[C@H](CO)[C@@H](O)[C@H](O)[C@H]1O)c1ccccc1 ; O=C([O-])C(F)(F)F ; O=C([O-])C(F)(F)F]

Prepared according to General Procedure D from FIM50 (1.5 mg, 1.00 μmol) in TFA (0.5 mL) and DCM (0.5 mL). Evaporation of solvents (1.5 mg, 0.95 μmol, 95%) gave the product as an orange solid.

MS (electrospray, +ve), [M]⁺ calculated for C₅₉H₇₆N₁₃O₂₀ requires: 1350.4766, found: 1350.5.

### Synthesis of I105

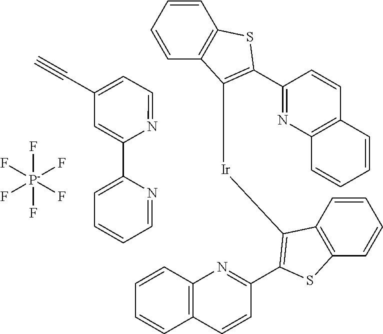

I106 (94.0 mg, 0.087 mmol) was dissolved in MeCN (3.5 mL) and THF (3.5 mL) containing AcOH (0.07 mL). To this was added tetrabutylammonium fluoride (0.22 mL of a 1 M solution) and the reaction mixture stirred for 18 h at RT. Ammonium hexafluorophosphate was added and the reaction concentrated. The residue was purified by RP MPLC (H₂O with increasing MeCN (0.1% formic acid)). Product obtained as an orange solid (65 mg, 0.061 mmol, 71%).

¹H NMR (400 MHz, Chloroform-d) δ 8.40-8.15 (m, 5H), 8.15-8.03 (m, 2H), 7.92 (d, J=8.6 Hz, 2H), 7.82 (d, J=8.1 Hz, 2H), 7.69 (d, J=8.0 Hz, 2H), 7.53 (t, J=6.6 Hz, 1H), 7.44 (dd, J=5.8, 1.6 Hz, 1H), 7.32-7.26 (m, 2H), 7.13 (td, J=7.6, 3.3 Hz, 2H), 6.98-6.78 (m, 4H), 6.62 (q, J=7.1 Hz, 2H), 6.28 (d, J=8.2 Hz, 1H), 6.23 (d, J=8.2 Hz, 1H), 3.59 (s, 1H).

### Synthesis of I106

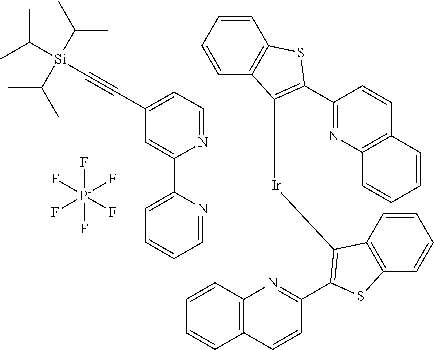

I107 (170 mg, 0.114 mmol) and I108 (77 mg, 0.227 mmol) were dissolved in 2-ethoxyethanol (30 mL) and heated at 115° C. for 60 h. The reaction was cooled, and the solvents removed under reduced pressure and the residue purified by MPLC (DCM with increasing MeOH). Product obtained as a red solid (94 mg, 0.087 mmol, 38%).

MS (electrospray, +ve), [M]⁺ calculated for C₅₅H₄₈IrN₄S₂Si requires: 1049.2714, found: 1049.2.

¹H NMR (400 MHz, Chloroform-d) δ 8.54 (dd, J=37.3, 8.1 Hz, 2H), 8.32-8.15 (m, 5H), 7.91 (dd, J=8.6, 2.5 Hz, 2H), 7.82 (d, J=8.1 Hz, 2H), 7.74-7.58 (m, 3H), 7.40 (dd, J=5.9, 1.7 Hz, 1H), 7.35-7.26 (m, 2H), 7.13 (td, J=7.5, 4.8 Hz, 2H), 6.96 (d, J=8.9 Hz, 1H), 6.88 (dd, J=8.9, 2.9 Hz, 3H), 6.61 (dt, J=11.1, 7.8 Hz, 2H), 6.29 (d, J=8.2 Hz, 1H), 6.18 (d, J=8.2 Hz, 1H), 1.15-1.02 (m, 18H).

### Synthesis of I107

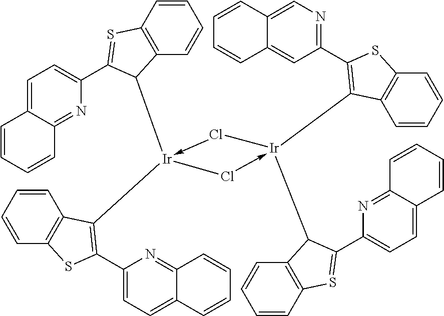

IrCl₃·H₂O (295 mg, 0.837 mmol) and I109 (468 mg, 1.790 mmol) were dissolved in 2-ethoxyethanol (60 mL) and water (30 mL) and the mixture heated at 115° C. for 24 h. The reaction mixture was cooled to room temperature and filtered. The solid was washed with water, giving the product as a green powder (491 mg, 0.328 mmol, 78%).

MS (electrospray, +ve), [M-2Cl]²⁺ calculated for C₆₈H₄₀Ir₂N₄S₄ requires: 712.0680, found: 712.0.

### Synthesis of I108

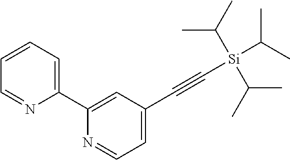 [SMILES: CC(C)[Si](C#Cc1ccnc(-c2ccccn2)c1)(C(C)C)C(C)C]

4-bromo-2,2′-bipyridine (500 mg, 2.13 mmol), bis(triphenylphosphine)palladium(II) dichloride (38 mg, 0.053 mmol) and copper iodide (38 mg, 0.197 mmol) stirred in degassed THF (3 mL). (Triisopropylsilyl)acetylene (0.763 mL, 3.403 mmol) added, followed by Et₃N (5 mL) and the reaction was heated to 50° C. for 2 h, then left at RT for 16 h. Solvents removed under reduced pressure and the residue dry loaded onto silica and purified by MPLC (Et₂O with a drop of Et₃N). Product obtained as a brown oil (524 mg, 1.56 mmol, 73%).

¹H NMR (400 MHz, Chloroform-d) δ 8.76-8.65 (m, 1H), 8.62 (d, J=5.0 Hz, 1H), 8.43 (s, 1H), 8.38 (d, J=8.0 Hz, 1H), 7.83 (td, J=7.7, 1.8 Hz, 1H), 7.43-7.29 (m, 2H), 1.14 (s, 18H).

### Synthesis of I109

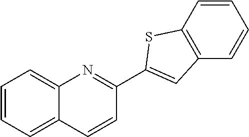 [SMILES: c1ccc2nc(-c3cc4ccccc4s3)ccc2c1]

2-chloroquinine (491 mg, 3.00 mmol) and benzo[b]thiophen-2-ylboronic acid (587 mg, 3.30 mmol) dissolved in THF (30 mL) and water (30 mL) and K₂CO₃ (1.25 g, 9.00 mmol) and tetrakis(triphenylphosphine)palladium(0) (277 mg, 0.24 mmol) were added. The reaction was stirred at 75° C. for 4 h. THF was removed under a stream of N₂ and the remaining aqueous extracted with DCM (3×30 mL). The combined organics were washed with water (30 mL), dried (Na₂SO₄) and concentrated under reduced pressure and purified by MPLC (DCM:Petrol, 1:1). Product obtained as a white solid (471 mg, 1.80 mmol, 60%).

MS (electrospray, +ve), [M+H]⁺ calculated for C₁₇H₁₂NS requires: 262.0685, found: 262.0.

¹H NMR (400 MHz, Chloroform-d) δ 8.19 (dd, J=8.6, 0.8 Hz, 1H), 8.14 (dq, J=8.6, 0.9 Hz, 1H), 7.98 (d, J=0.8 Hz, 1H), 7.95 (d, J=8.6 Hz, 1H), 7.93-7.88 (m, 1H), 7.88-7.83 (m, 1H), 7.83-7.79 (m, 1H), 7.73 (ddd, J=8.5, 6.9, 1.5 Hz, 1H), 7.53 (ddd, J=8.1, 6.9, 1.2 Hz, 1H), 7.42-7.34 (m, 2H).

### I110

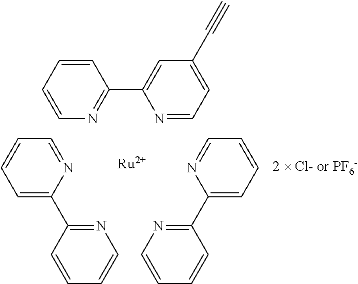

As prepared in Z. Anorq. Allq. Chem., 646: 842-848

### Synthesis of I111

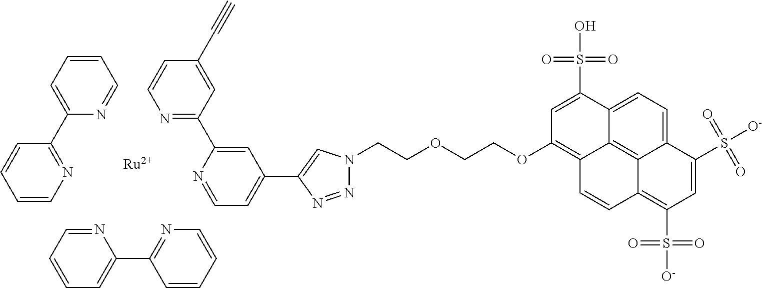

Prepared according to General Procedure B from I112 (12 mg, 17.4 μmol), I113 (22 mg, 18.8 μmol), CuSO₄·5H₂O (0.3 mg, 1.7 μmol) and sodium ascorbate (25 mg, 126 μmol) in water (1.5 mL) and THF (1 mL). Solvents removed and the residue purified by RP MPLC (H₂O with increasing MeCN (0.1% formic acid)). Product obtained as a yellow solid (8 mg, 6.1 μmol, 35%).

MS (electrospray, +ve), [M+H]⁺ calculated for C₅₄H₄₀N₉O₁₁RuS₃ requires: 1188.1048, found: 1188.1.

¹H NMR (400 MHz, Deuterium Oxide with Methanol-d₄) δ 9.23 (s, 1H), 8.99 (d, J=9.6 Hz, 1H), 8.86 (d, J=9.9 Hz, 1H), 8.76 (d, J=9.7 Hz, 1H), 8.61 (s, 1H), 8.57-8.46 (m, 3H), 8.37-8.28 (m, 3H), 8.25 (s, 2H), 8.20-8.10 (m, 1H), 8.03 (dt, J=16.8, 7.9 Hz, 2H), 7.90 (t, J=8.0 Hz, 1H), 7.63 (dd, J=17.2, 5.7 Hz, 3H), 7.55 (q, J=5.8, 5.3 Hz, 2H), 7.42-7.33 (m, 2H), 7.30 (s, 1H), 7.24 (dd, J=10.2, 6.2 Hz, 2H), 7.11 (s, 1H), 7.00 (t, J=6.7 Hz, 1H), 4.44 (s, 2H), 4.19 (s, 2H), 4.10 (s, 1H), 4.03 (s, 2H), 1.33 (dd, J=6.8, 4.1 Hz, 1H).

### Synthesis of I112

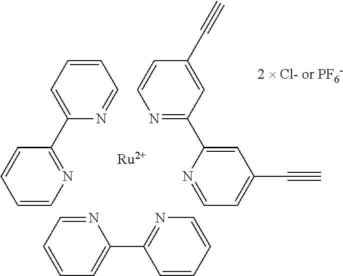

As prepared in Z. Anorq. Allq. Chem., 646: 842-848

### Synthesis of I113

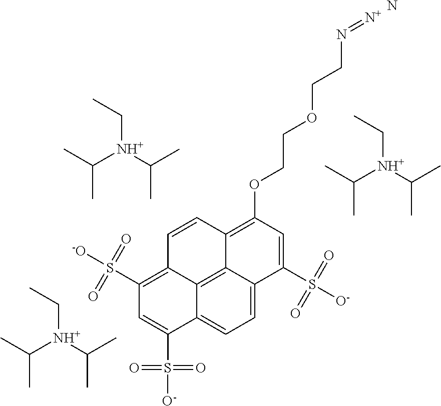

To a solution of pyranine (100 mg, 0.22 mmol) in 80° C. MeOH (10 mL) was added I48 (187 mg, 0.65 mmol), NaI (33 mg, 0.22 mmol) and DIPEA (0.38 mL, 2.18 mmol) and the reaction was stirred for 20 h. The MeOH was removed under a stream of N₂ and the residue suspended in water and the aqueous solution purified by RP MPLC (H₂O with increasing MeCN (0.1% formic acid)). Product, with 17% tosic acid impurity, obtained as a yellow solid (45 mg, 0.039 mmol, 18%).

MS (electrospray, -ve), [M−H]⁻ calculated for C₂₀H₁₆N₃O₁₁S₃ requires: 569.9952, found: 569.9.

¹H NMR (400 MHz, Deuterium Oxide) δ 9.04 (d, J=0.5 Hz, 1H), 9.01 (d, J=9.8 Hz, 1H), 8.91 (d, J=9.5 Hz, 1H), 8.84 (d, J=9.8 Hz, 1H), 8.74 (d, J=9.6 Hz, 1H), 8.26 (s, 1H), 7.54 (d, J=8.3 Hz, 1H), 7.24-7.20 (m, 1H), 4.55 (ddd, J=5.3, 2.7, 1.4 Hz, 2H), 4.01 (dd, J=5.3, 3.1 Hz, 2H), 3.77-3.72 (m, 2H), 2.25 (s, 2H).

### Synthesis of I114

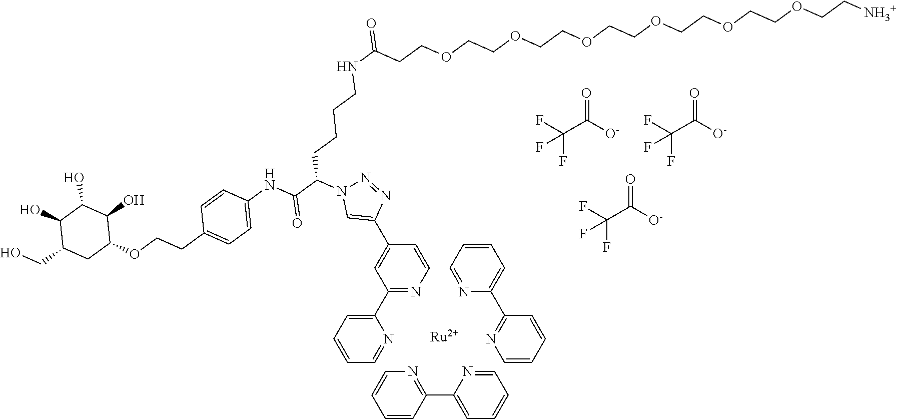

Prepared according to General Procedure D from FIM58 (11 mg, 7.0 μmol) in TFA (0.3 mL) and DCM (2 mL). Product obtained as an orange solid (5 mg, 3.5 μmol, 50%).

MS (electrospray, +ve) [M]²⁺ calculated for C₆₇H₈₄N₁₂O₁₄Ru requires: 691.2631, found: 691.3.

### Synthesis of I115

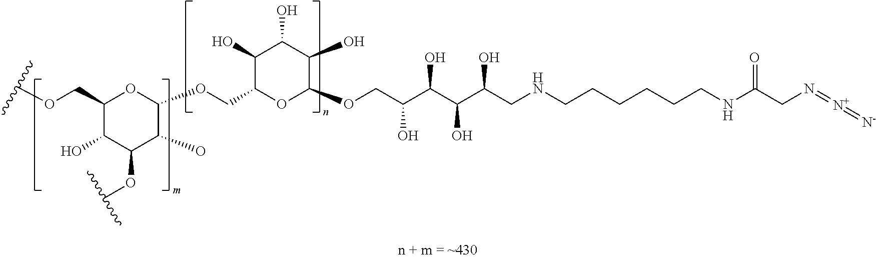

Monoamino dextran MW 70 kDa (56 mg, 0.80 μmol) and NaHCO₃ (2.4 mg, 28 μmol) were dissolved in water with 0.02% NaN₃ (1 mL). Azidoacetic acid NHS ester (3 mg, 16.0 μmol) was added and the reaction stirred for 7 h. The modified dextran was precipitated with EtOH and the white solid isolated by centrifugation (38 mg, 0.54 μmol, 68%).

### Synthesis of I116

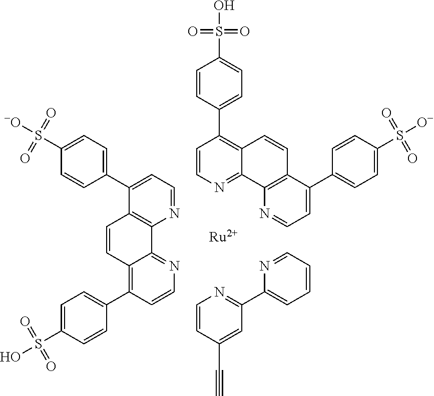

I121 (50 mg, 0.035 mmol) was dissolved in MeOH (1 mL) and THF (2 mL) containing AcOH (0.03 mL). To this was added tetrabutylammonium fluoride (0.6 mL of a 1 M solution) and the reaction mixture stirred for 18 h at RT. Ammonium hexafluorophosphate was added and the reaction concentrated. The residue was purified by RP MPLC (H₂O with increasing MeCN (0.1% formic acid)). Product obtained as an orange solid (31 mg, 0.023 mmol, 65%).

MS (electrospray, +ve) [M+2H]²⁺ calculated for C₆₀H₄₀N₆O₁₂RuS₄ requires: 633.0310, found: 633.1.

### Synthesis of I117

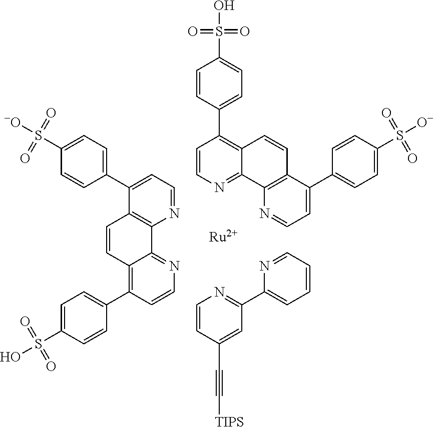

RuCl₃ (15 mg, 0.072 mmol), bathophenanthrolinedisulfonic acid, disodium salt hydrate (85 mg, 0.145 mmol), I108 (24 mg, 0.072 mmol) and LiCl (61 mg, 1.45 mmol) were dissolved in 2-ethoxyethanol (2 mL) and water (0.01 mL) and the mixture heated in a sealed microwave vial at 135° C. for 24 h. The reaction mixture was cooled to room temperature and concentrated. The residue was purified by RP MPLC (H₂O with increasing MeCN (0.1% formic acid)). Product obtained as an orange solid (50 mg, 0.035 mmol, 49%).

MS (electrospray, +ve) [M+2H]²⁺ calculated for C₆₉H₆₀N₆O₁₂RuS₄Si requires: 711.0977, found: 711.2.

### Synthesis of I118

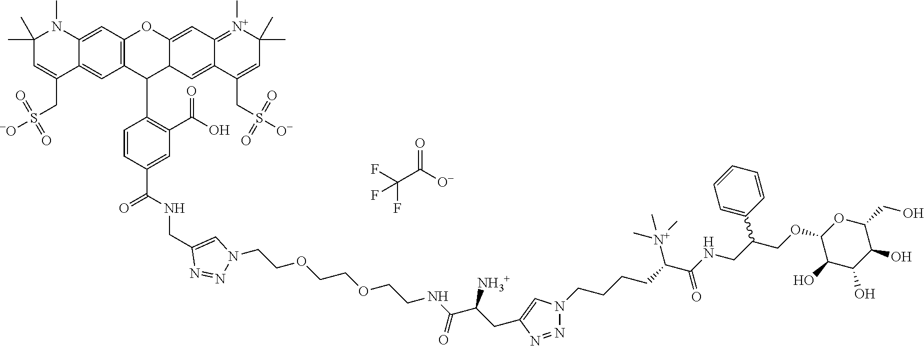

Prepared according to General Procedure D from I137 (3.3 mg, 2.0 μmol) in TFA (0.5 mL) and DCM (0.5 mL). Product obtained as a blue solid (3.3 mg, 2.0 μmol, 100%).

### Synthesis of I119

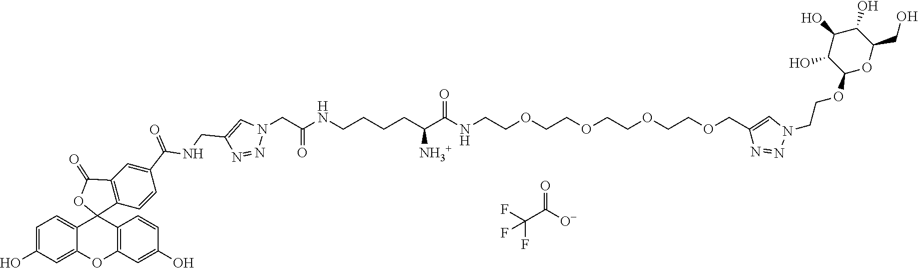 [SMILES: [NH3+][C@@H](CCCCNC(=O)Cn1cc(CNC(=O)c2ccc3c(c2)C(=O)OC32c3ccc(O)cc3Oc3cc(O)ccc32)nn1)C(=O)NCCOCCOCCOCCOCc1cn(CCO[C@@H]2O[C@H](CO)[C@@H](O)[C@H](O)[C@H]2O)nn1 ; O=C([O-])C(F)(F)F]

Prepared according to General Procedure 0 from I120 (10 mg, 8.3 μmol) in TFA (0.25 mL) and DCM (1.0 mL). Product obtained as an orange solid (10 mg, 8.2 μmol, 99%).

MS (electrospray, +ve), [M+H]⁺ calculated for C₉₁H₆₅N₁₀O₁₈ requires: 1105.4473, found: 1105.4.

### Synthesis of I120

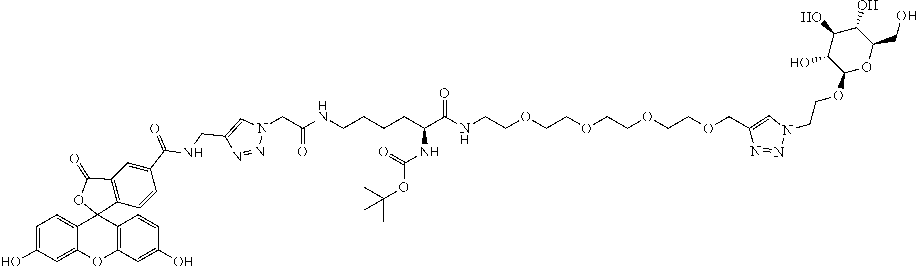 [SMILES: CC(C)(C)OC(=O)N[C@@H](CCCCNC(=O)Cn1cc(CNC(=O)c2ccc3c(c2)C(=O)OC32c3ccc(O)cc3Oc3cc(O)ccc32)nn1)C(=O)NCCOCCOCCOCCOCc1cn(CCO[C@@H]2O[C@H](CO)[C@@H](O)[C@H](O)[C@H]2O)nn1]

Prepared according to General Procedure B from I121 (10 mg, 0.013 mmol), 5-FAM alkyne (6 mg, 0.015 mmol), CuSO₄·5H₂O (3 mg, 0.015 mmol), sodium ascorbate (7.5 mg, 0.038 mmol) and Na₂CO₃ (4 mg, 0.038 mmol) in water (1 mL) and THF (1 mL). Solvents removed and the residue purified by RP MPLC (H₂O with increasing MeCN (0.1% formic acid)). Product obtained as an orange solid (12 mg, 9.96 μmol, 77%).

MS (electrospray, +ve), [M+H]⁺ calculated for C₅₆H₇₃N₁₀O₂₀ requires: 1205.4998, found: 1205.4.

### Synthesis of I121

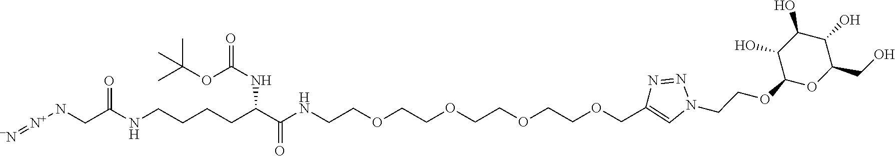

Prepared according to General Procedure C from I122 (54 mg, 0.076 mmol), azidoacetic acid NHS ester (30 mg, 0.152 mmol) and NaHCO₃ (13 mg, 0.152 mmol) in MeCN (8 mL) and water (4 mL). Purified by RP MPLC (H₂O with increasing MeCN (0.1% formic acid)). Product obtained as a white solid (60 mg, 0.076 mmol, 100%).

MS (electrospray, +ve), [M+Na]⁺ calculated for C₃₂H₅₇N₉NaO₁₄ requires: 814.3918, found: 814.4.

### Synthesis of I122

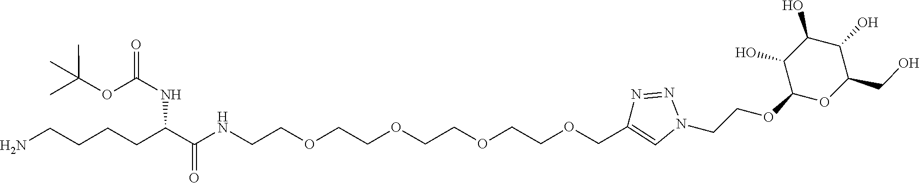 [SMILES: CC(C)(C)OC(=O)N[C@@H](CCCCN)C(=O)NCCOCCOCCOCCOCc1cn(CCO[C@@H]2O[C@H](CO)[C@@H](O)[C@H](O)[C@H]2O)nn1]

To a solution of I123 (400 mg, 0.430 mmol) in DCM (20 mL) was added DBU (0.19 mL, 1.29 mmol). Reaction stirred for 2 h at RT before solvents were removed under vacuum. Purified by RP MPLC (H₂O with increasing MeCN (0.1% formic acid)). Product obtained as a clear oil (54 mg, 0.076 mmol, 18%).

MS (electrospray, +ve) [M+H]⁺ calculated for C₃₀H₅₇N₆O₁₃ requires: 709.3979, found: 709.4.

### Synthesis of I123

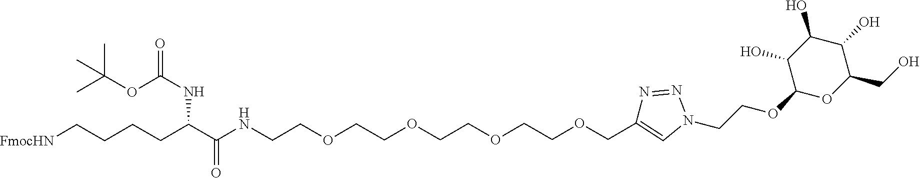

Prepared according to General Procedure A from Na-Boc-Nε-Fmoc-L-lysine (535 mg, 1.141 mmol), I124 (457 mg, 0.951 mmol), HBTU (721 mg, 1.902 mmol), HOBt·H2O (291 mg, 1.902 mmol) and DIPEA (0.33 mL, 1.902 mmol) in DMF (10 mL). Purified by RP MPLC (H₂O with increasing MeCN (0.1% formic acid)). Product obtained as a clear oil (415 mg, 0.446 mmol, 47%).

MS (electrospray, +ve) [M+H]⁺ calculated for C₄₅H₆₇N₆O₁₅ requires: 931.4659, found: 931.4.

### Synthesis of I124

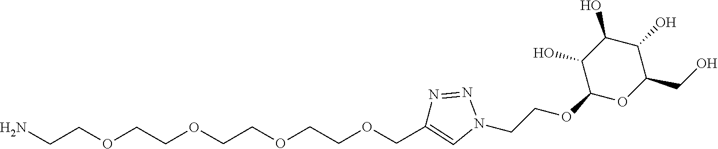 [SMILES: NCCOCCOCCOCCOCc1cn(CCO[C@@H]2O[C@H](CO)[C@@H](O)[C@H](O)[C@H]2O)nn1]

Prepared according to General Procedure B from (2R,3R,4S,5R,6R)-2-(acetoxymethyl)-6-(2-azidoethoxy)tetrahydro-2H-pyran-3,4,5-triyl triacetate (720 mg, 1.725 mmol), 3,6,9,12-tetraoxapentadec-14-yn-1-amine (599 mg, 2.588 mmol), CuSO₄·5H₂O (172 mg, 0.690 mmol) and sodium ascorbate (410 mg, 2.07 mmol) in water (8 mL) and THF (30 mL). After 16 h, NaOH (327 mg, 8.17 mmol) was added and stirred for 1 h. Solvents removed, and the residue purified by RP MPLC (H₂O with increasing MeCN (0.1% formic acid)). Product obtained as a clear oil (535 mg, 1.11 mmol, 66%).

¹H NMR (400 MHz, Deuterium Oxide) δ 8.03 (s, 1H), 4.61-4.53 (m, 4H), 4.29 (d, J=7.9 Hz, 1H), 4.18 (dt, J=10.1, 4.8 Hz, 1H), 3.99 (dt, J=11.0, 5.0 Hz, 1H), 3.75 (dd, J=12.3, 2.2 Hz, 1H), 3.67-3.58 (m, 2H), 3.61-3.47 (m, 12H), 3.28 (s, 1H), 3.36-3.16 (m, 3H), 3.13-3.03 (m, 3H).

### Synthesis of I125

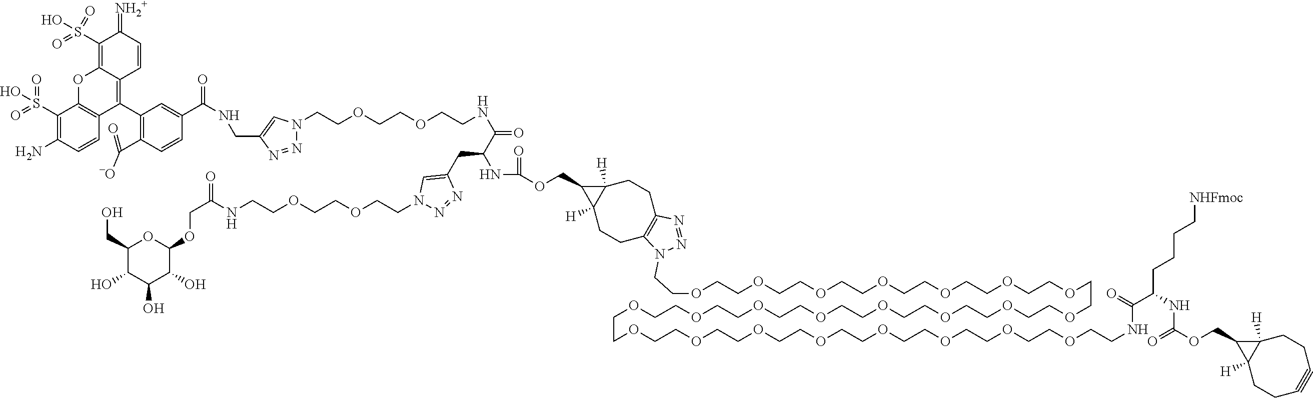

Prepared according to General Procedure C from I126 (1 mg, 0.344 μmol), (1R,8S,9s)-bicyclo[6.1.0]non-4-yn-9-ylmethyl N-succinimidyl carbonate (1 mg, 3.4 μmol) and NaHCO₃ (5 mg, 57.1 μmol) in MeCN (0.2 mL) and water (0.2 mL). Purified by RP MPLC (H₂O with increasing MeCN (0.1% formic acid)). Product obtained as an orange solid (1 mg, 0.329 μmol, 96%).

MS (electrospray, +ve) [M+3H]³⁺ calculated for C₁₄₀H₂₀₉N₁₈O₅₂S₂ requires: 1013.1241, found 1013.2.

### Synthesis of I126

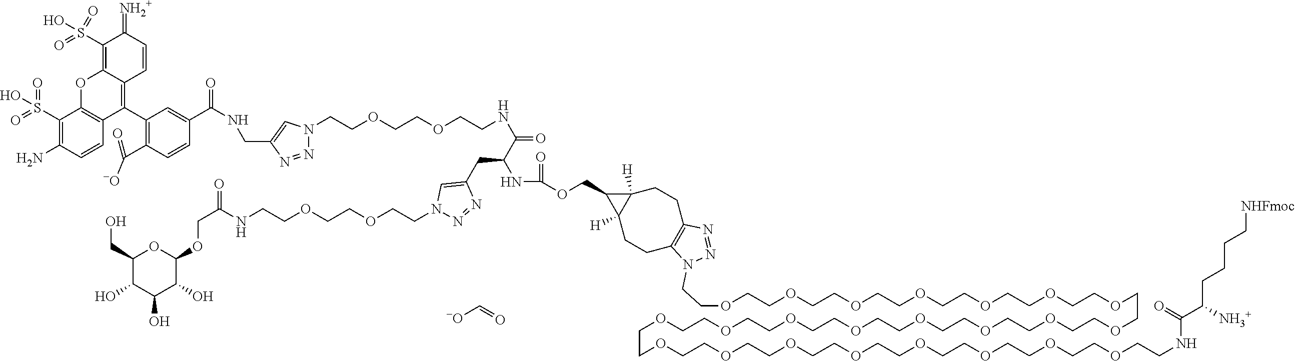

I127 (1 mg, 0.338 μmol) dissolved in formic acid (1 mL) and stirred for 16 h. Formic acid removed under a stream of N₂. Product obtained as an orange solid (1 mg, 0.344 μmol, 100%)

MS (electrospray, +ve) [M+2H]²⁺ calculated for C₁₂₉H₁₉₆N₁₃O₅₀S₂ requires: 1431.1406, found 1431.2.

### Synthesis of I127

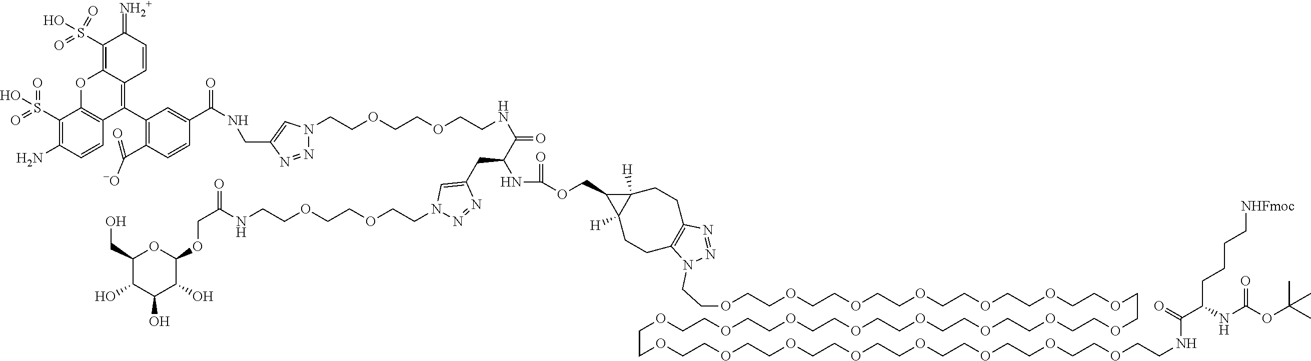

I128 (1.2 mg, 0.850 μmol) and I129 (3 mg, 1.9 μmol) were dissolved in water (0.5 mL) and stirred for 18 h. Purified by RP MPLC (H₂O with increasing MeCN (0.1% formic acid)). Product obtained as an orange solid (1 mg, 0.338 μmol, 40%).

MS (electrospray, +ve) [M+2H]²⁺ calculated for C₁₃₄H₂₀₄N₁₈O₅₂S₂ requires: 1481.1668, found 1481.2.

### Synthesis of I128

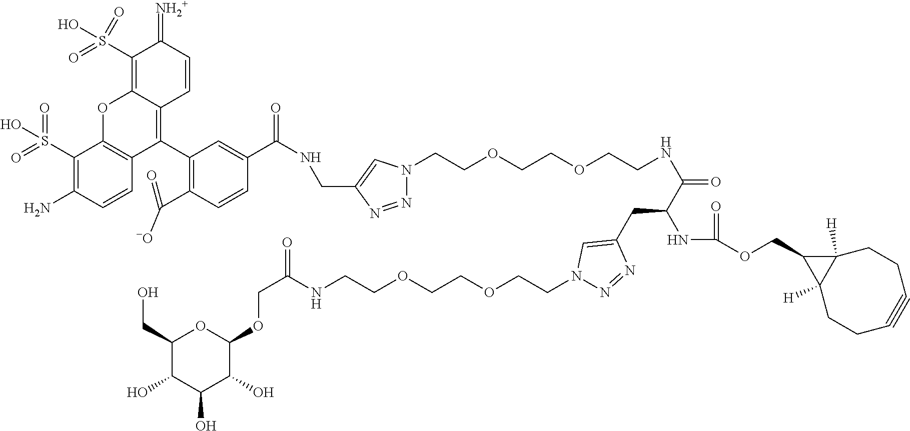

Prepared according to General Procedure C from I130 (6.6 mg, 4.9 μmol), (1R,8S,9s)-bicyclo[6.1.0]non-4-yn-9-ylmethyl N-succinimidyl carbonate (19 mg, 65.2 μmol) and NaHCO₃ (29 mg, 0.347 mmol) in MeCN (4 mL) and water (4 mL). Purified by RP MPLC (H₂O with increasing MeCN (0.1% formic acid)). Product obtained as an orange solid (3.3 mg, 2.3 μmol, 48%).

MS (electrospray, +ve) [M+H]⁺ calculated for C₆₀H₇₅N₁₂O₂₄S₂ requires: 1411.4454, found 1411.4.

### Synthesis of I129

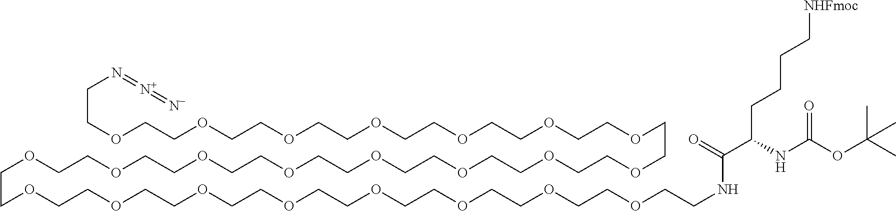

Prepared according to General Procedure A from Na-Boc-Nε-Fmoc-L-lysine (27 mg, 58.2 μmol), azido-PEG₂₃-amine (32 mg, 29.1 μmol), HATU (28 mg, 73.6 μmol) and DIPEA (0.02 mL, 116 μmol) in MeCN (3.5 mL). Purified by MPLC (EtOAc/MeOH with increasing MeOH) then RP MPLC (H₂O with increasing MeOH). Product obtained as a yellow gum (38 mg, 24.5 μmol, 84%).

¹H NMR (400 MHz, Methanol-d₄) δ 7.80 (dd, J=7.7, 4.7 Hz, 2H), 7.65 (d, J=7.5 Hz, 2H), 7.40 (t, J=7.4 Hz, 2H), 7.36-7.26 (m, 2H), 4.35 (t, J=8.0 Hz, 2H), 4.20 (t, J=6.8 Hz, 1H), 3.99 (s, 1H), 3.75-3.54 (m, 90H), 3.52 (t, J=5.4 Hz, 2H), 3.37 (p, J=9.5, 7.7 Hz, 4H), 3.11 (q, J=6.5 Hz, 2H), 1.83-1.48 (m, 4H), 1.43 (s, 9H), 1.39-1.06 (m, 2H).

### Synthesis of I130

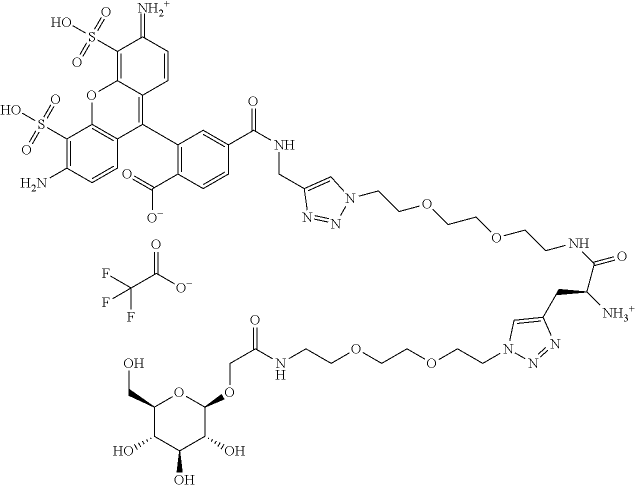 [SMILES: Nc1ccc2c(-c3cc(C(=O)NCc4cn(CCOCCOCCNC(=O)[C@@H]([NH3+])Cc5cn(CCOCCOCCNC(=O)CO[C@@H]6O[C@H](CO)[C@@H](O)[C@H](O)[C@H]6O)nn5)nn4)ccc3C(=O)[O-])c3ccc(=[NH2+])c(S(=O)(=O)O)c-3oc2c1S(=O)(=O)O ; O=C([O-])C(F)(F)F]

Prepared according to General Procedure D from I131 (8 mg, 6.0 μmol) in TFA (1 mL) and DCM (2.3 mL). Product obtained as a purple solid (7.4 mg, 5.5 μmol, 92%).

MS (electrospray, +ve), [M+H]⁺ calculated for C₄₉H₆₃N₁₂O₂₂S₂ requires: 1235.3616, found: 1235.3.

### Synthesis of I131

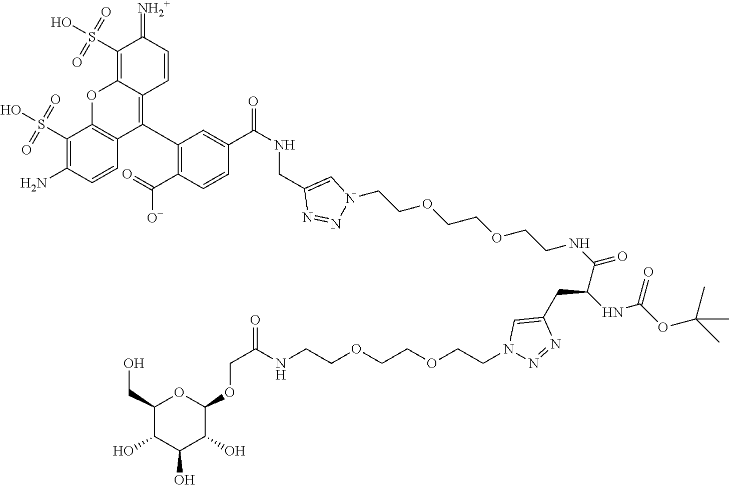 [SMILES: CC(C)(C)OC(=O)N[C@@H](Cc1cn(CCOCCOCCNC(=O)CO[C@@H]2O[C@H](CO)[C@@H](O)[C@H](O)[C@H]2O)nn1)C(=O)NCCOCCOCCn1cc(CNC(=O)c2ccc(C(=O)[O-])c(-c3c4ccc(=[NH2+])c(S(=O)(=O)O)c-4oc4c(S(=O)(=O)O)c(N)ccc34)c2)nn1]

Prepared according to General Procedure B from I132 (16 mg, 20.7 μmol), 5-FAM alkyne (5 mg, 8.7 μmol), CuSO₄·5H₂O (2 mg, 7 μmol) and sodium ascorbate (9 mg, 43.9 μmol) in water (0.7 mL). Solvents removed and the residue purified by RP MPLC (H₂O with increasing MeCN (0.1% formic acid)). Product obtained as an orange solid (8 mg, 6.0 μmol, 69%).

MS (electrospray, +ve), [M+H]⁺ calculated for C₅₄H₇₁N₁₂O₂₄S₂ requires: 1335.4140, found: 1335.3.

### Synthesis of I132

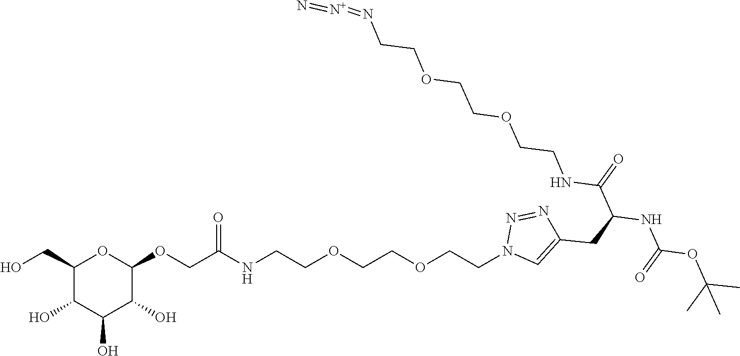 [SMILES: CC(C)(C)OC(=O)N[C@@H](Cc1cn(CCOCCOCCNC(=O)CO[C@@H]2O[C@H](CO)[C@@H](O)[C@H](O)[C@H]2O)nn1)C(=O)NCCOCCOCCN=[N+]=N]

Prepared according to General Procedure A from I133 (242 mg, 0.398 mmol), azido-PEG₂-amine (260 mg, 1.493 mmol), HATU (235 mg, 0.618 mmol) and DIPEA (0.208 mL, 1.195 mmol) in DMF (2 mL). Purified by RP MPLC (H₂O with increasing MeCN (0.1% formic acid)). Product obtained as a brown gum (169 mg, 0.221 mmol, 56%).

MS (electrospray, +ve), [M+H]⁺ calculated for C₃₀H₅₄N₉O₁₄ requires: 764.3785, found: 764.3.

¹H NMR (400 MHz, Deuterium Oxide) δ 7.76 (s, 1H), 4.45 (t, J=5.0 Hz, 2H), 4.35 (d, J=7.9 Hz, 1H), 4.26-4.15 (m, 2H), 4.11 (d, J=15.7 Hz, 1H), 3.81 (t, J=5.0 Hz, 2H), 3.75 (dd, J=12.3, 2.0 Hz, 1H), 3.60-3.40 (m, 16H), 3.39-3.17 (m, 11H), 3.06 (d, J=11.4 Hz, 1H), 2.90 (dd, J=14.9, 9.1 Hz, 1H), 1.23 (s, 9H).

### Synthesis of I133

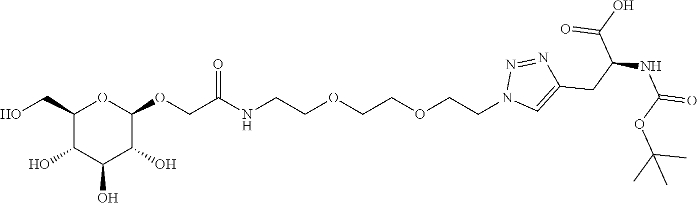 [SMILES: CC(C)(C)OC(=O)N[C@@H](Cc1cn(CCOCCOCCNC(=O)CO[C@@H]2O[C@H](CO)[C@@H](O)[C@H](O)[C@H]2O)nn1)C(=O)O]

Prepared according to General Procedure B from I134 (164 mg, 0.416 mmol), boc-L-2-propargylglycine (124 mg, 0.580 mmol), CuSO₄·5H₂O (52 mg, 0.208 mmol) and sodium ascorbate (247 mg, 1.248 mmol) in water (5.4 mL) and THF (3 mL). Solvents removed and the residue purified by RP MPLC (H₂O with increasing MeCN (0.1% formic acid)). Product obtained as a white solid (247 mg, 0.417 mmol, 98%).

MS (electrospray, +ve), [M+H]⁺ calculated for C₂₄H₄₂N₅O₁₃ requires: 608.2774, found: 608.3.

¹H NMR (400 MHz, Deuterium Oxide) δ 4.47 (s, 2H), 4.35 (d, J=7.7 Hz, 1H), 4.23 (d, J=15.7 Hz, 1H), 4.11 (d, J=15.6 Hz, 1H), 3.84 (d, J=7.6 Hz, 2H), 3.76 (d, J=12.3 Hz, 1H), 3.58 (dd, J=12.4, 5.3 Hz, 1H), 3.47 (s, 4H), 3.42 (d, J=5.2 Hz, 2H), 3.40-3.17 (m, 7H), 3.07 (s, 1H), 2.86 (s, 1H), 1.24 (s, 9H).

### Synthesis of I134

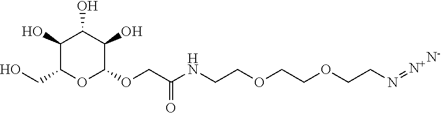

I135 (300 mg, 0.533 mmol) dissolved in MeOH (3 mL) and sodium methoxide (10 mg, 0.187 mmol) was added. The reaction was stirred for 30 min then neutralised with 1 M aq. HCl. The solvent was removed, and the crude product purified by MPLC (DCM with increasing MeOH). Product obtained as a colourless gum.

¹H NMR (400 MHz, Methanol-d₄) δ 4.34-4.28 (m, 2H), 4.15 (d, J=15.7 Hz, 1H), 3.87 (dd, J=12.0, 1.7 Hz, 1H), 3.71-3.62 (m, 7H), 3.60 (t, J=5.5 Hz, 2H), 3.49-3.42 (m, 2H), 3.42-3.35 (m, 3H), 3.30-3.24 (m, 2H).

### Synthesis of I135

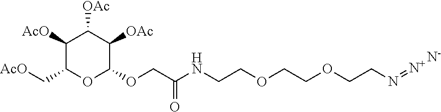

Prepared according to General Procedure A from I136 (300 mg, 0.738 mmol), azido-PEG₂-amine (167 mg, 0.960 mmol), HBTU (336 mg, 0.886 mmol), HOBt·H2O (50 mg, 0.325 mmol) and DIPEA (0.26 mL, 1.48 mmol) in DMF (5 mL). The reaction was diluted with EtOAc (20 mL) and washed with 0.1 M aq. HCl (20 mL), water (20 mL) and brine (20 mL). The organic layer was dried (MgSO₄) and evaporated to give the crude product. Purified by MPLC (EtOAc). Product obtained as a clear gum, containing some HOBt, which was used without further purification (300 mg, 0.533 mmol, 72%).

### Synthesis of I136

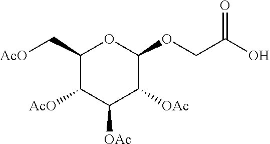 [SMILES: CC(=O)OC[C@H]1O[C@@H](OCC(=O)O)[C@H](OC(C)=O)[C@@H](OC(C)=O)[C@@H]1OC(C)=O]

As prepared in Tetrahedron Lett. 2016, 57: 1425-1429.

### Synthesis of I137

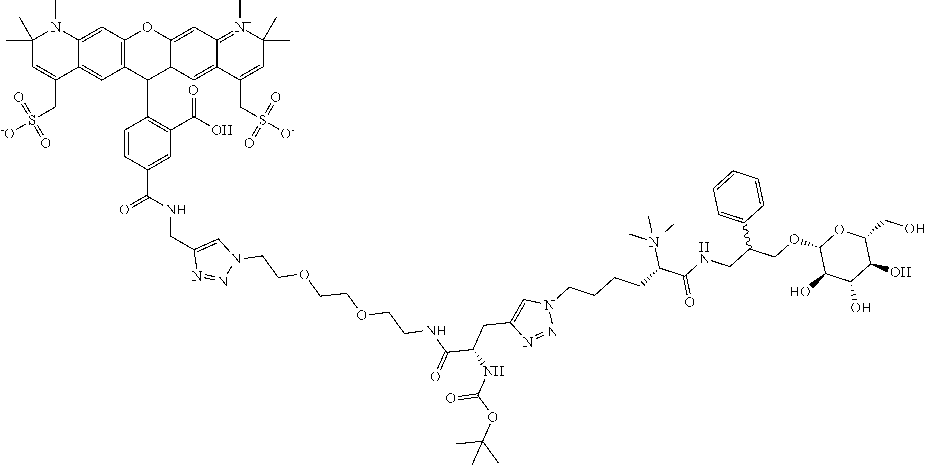

Prepared according to General Procedure B from I89 (2.3 mg, 2.1 μmol), AF594-alkyne (1 mg, 1.0 μmol), CuSO₄·5H₂O (0.2 mg, 0.8 μmol) and sodium ascorbate (0.4 mg, 2.1 μmol) in water (0.4 mL) and MeOH (0.2 mL). Solvents removed and the residue purified by RP MPLC (H₂O with increasing MeCN (0.1% formic acid)) to give the product as a purple solid (1.7 mg, 1.0 μmol, 100%).

### Other Compounds

“Ru(bpy)3 alkyne”

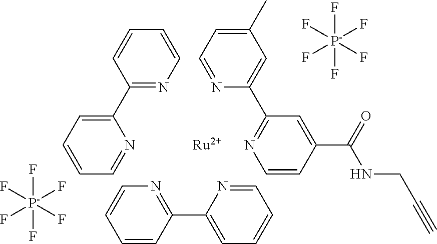

As prepared in Inorq. Chem. 1999, 38, 2411-2415

### “Pyranine-alkyne”

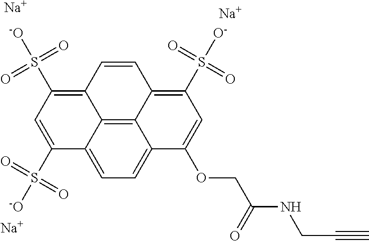

Commonly known as AFdye 405 alkyne (or AF405 alkyne) which is commercially available.

### Glucose Responsive Assays

### General Procedures for Determination of Glucose Responsive Assays

### Fluorescent Solution Phase

A solution of GBM and FIM at the required concentrations were created in the appropriate media. The emission spectrum of the assay was then acquired by exciting at the designated excitation wavelength to obtain the baseline emission intensity for the assay. Aliquots of 2 M D-glucose (in the same media) were then added to give the required final glucose concentrations (e.g. 15 μL of 2 M D-glucose in 3 mL gives a final D-glucose concentration of 10 mM) and the assay allowed to reach equilibration. The emission spectra were collected after each addition of D-glucose and compared to the original baseline emission, monitoring at the typical emission maximum (Emₘₐₓ), and the percentage increase or decrease recorded. Emission spectra acquired on either a Horiba Duetta fluorometer or Clariostar plus plate reader.

### Fluorescent Hydrogel Phase

The fluorescence spectrum of the corresponding hydrogel, already swollen in 200 μL of 10 mM PBS, in a well plate was recorded by exciting at the designated excitation frequency and monitoring at the typical emission maximum (Emₘₐₓ). The PBS top solution was removed either by micropipette or by decanting onto an absorbent material. A 200 μL solution of D-glucose (in 10 mM PBS) at the required concentration was added to each hydrogel in the well plate and the system allowed to reach equilibrium. The emission spectra for each well were collected after this and compared to the original baseline emission, monitoring at the typical emission maximum (Emₘₐₓ), and the percentage increase or decrease recorded. The change in emission was recorded for each hydrogel in the well plate (up to 96 individual hydrogel sensors) or an average of all wells was used. The hydrogel bound assays were then investigated for reproducibility by cycling with solutions of PBS or D-glucose. This was achieved by removal of the previous top solution (e.g. D-glucose) by micropipette or decanting onto an absorbent material and replacing with another solution (e.g. 10 mM PBS). This process was repeated to up to 10 times to demonstrate reversibility of the hydrogel bound assay. Emission spectra acquired using a Clariostar plus plate reader.

### Fluorescence Lifetime

Separate samples containing a mixture of GBM, FIM and D-glucose at various concentrations in 10 mM PBS were prepared in 4-sided quartz cuvettes. These cuvettes were then subjected to fluorescence lifetime measurements using multi-channel scanning (MCS) using the appropriate excitation wavelength and monitoring at the expected emission maximum (Emₘₐₓ). The data were fitted to the sum of exponentials, with the goodness of fit determined by a chi-squared value and weighted residuals. Time per bin width used was 5 ns. Comparison of the average fluorescence lifetime for each sample versus the amount of D-glucose present in each sample gave the D-glucose responsive fluorescence lifetime for the tested assay. Fluorescence lifetime measurements were performed using a HORIBA DeltaFlex-01-DD.

### Affinity Electrochemistry—Cyclic Voltammetry

Separate samples containing a mixture of GBM, FIM and D-glucose at various concentrations in 10 mM PBS were prepared. 50 μL of each sample was pipetted onto the surface of the screen-printed electrode and the cyclic voltammogram acquired using the CV staircase protocol within the Nova software, using the following settings: Start potential at −0.4 V, upper vertex potential at 0.6 V, lower vertex potential at −0.4 V, stop potential at −0.39756 V, number of scans=3, scan rate at 0.1 V/s and Step at 0.00244 V. The data for each voltammogram was exported into Microsoft excel, processed and compared to give the glucose responsive electrochemical results.

### Results of Fluorescent Solution Phase Glucose Responsive Assay

The glucose responsive fluorescence emission for different combinations of glucose binding molecules (GBM) and fluorescent inhibitor molecules (FIM) at various concentrations and in various media was tested as described above. The results are shown in Table 1. All results obtained at pH 7.4 and 298 K unless otherwise stated.

In addition, FIG. 3 shows an example glucose responsive fluorescence assay, featuring FIM4 (1 μM) and GBM2 (10 μM) in 10 mM PBS (pH 7.4, 298 K). Ex=470 nm, Emₘₐₓ=520 nm.

Table 2 shows the results for glucose responsive fluorescence emission, at various concentrations and in various media, for different glucose binding molecules (GBM) where the fluorescent inhibitor molecule (FIM) is covalently linked to the GBM. All results obtained at pH 7.4 and 298 K unless otherwise stated.

The glucose responsive fluorescence emission for different combinations of glucose binding molecules (GBM) and fluorescent inhibitor molecules (FIM) at various concentrations and in various media was tested as described above. The results are shown in Table 3. All results obtained at pH 7.4 and 298 K unless otherwise stated.

Table 4 provides the composition of the assay media used as the code assigned to each media as used in Tables 1 to 3.

TABLE 1

| | | | | | | | Change in Emₘₐₓ for FIM at a particular D-glucose | | | | | | |
|---|---|---|---|---|---|---|---|---|---|---|---|---|---|
| | | Ex/ | Emₘₐₓ/ | Assay | [GBM]/ | [FIM]/ | concentration/% | | | | | | |
| FIM | GBM | nm | nm | Medium | μM | μM | 5 mM | 10 mM | 20 mM | 40 mM | 60 mM | 80 mM | 100 mM |
| Carminic acid | GBM14 | 525 | 640 | A | 20 | 1 | | | 20% | | | | |
| Salidroside | GBM19 | 420 | 530 | B | 1 | 3000 | 5% | 10% | 20% | 40% | | | |
| FIM1 | GBM1 | 470 | 520 | B | 10 | 1 | | | 50% | | 90% | 110% | |
| FIM1 | GBM1 | 470 | 520 | J | 10 | 1 | | | 30% | 35% | | | |
| FIM1 | GBM1 | 470 | 520 | K | 10 | 1 | | | 50% | 75% | 90% | 110% | |
| FIM2 | GBM16 | 550 | 590 | B | 10 | 1 | | | 75% | | | | |
| FIM2 | GBM14 | 550 | 590 | B | 10 | 1 | | | 5% | | | | |
| FIM2 | GBM15 | 550 | 590 | B | 10 | 1 | | | 25% | | | | |
| FIM2 | GBM13 | 550 | 590 | B | 10 | 1 | | | 5% | | | | |
| FIM2 | GBM4 | 550 | 590 | B | 10 | 1 | | | 20% | | | | |
| FIM2 | GBM20 | 550 | 590 | B | 10 | 1 | | | 20% | | | | |
| FIM2 | GBM16 | 550 | 590 | C | 50 | 5 | | | 70% | | | | |
| FIM2 | GBM15 | 550 | 590 | C | 50 | 5 | | | 10% | | | | |
| FIM2 | GBM13 | 550 | 590 | C | 50 | 5 | | | 5% | | | | |
| FIM3 | GBM15 | 470 | 520 | B | 10 | 1 | | 270% | 280% | 300% | | | |
| FIM3 | GBM15 | 470 | 520 | B | 25 | 5 | 270% | 280% | 300% | 310% | | | |
| FIM3 | GBM15 | 470 | 520 | B | 100 | 1 | 150% | 195% | 240% | 280% | | | |
| FIM3 | GBM7 | 470 | 520 | B | 10 | 1 | 250% | 260% | 285% | 305% | | | |
| FIM3 | GBM7 | 470 | 520 | B | 30 | 1 | | 230% | 250% | 270% | | | |
| FIM4 | GBM3 | 470 | 520 | E | 10 | 1 | | | 20% | | | | |
| FIM4 | GBM3 | 470 | 520 | F | 10 | 1 | | | 45% | 60% | 70% | | |
| FIM4 | GBM3 | 470 | 520 | G | 10 | 1 | | | 75% | 90% | 100% | | |
| FIM4 | GBM3 | 470 | 520 | H | 10 | 1 | | | 75% | 90% | 100% | | |
| FIM4 | GBM2 | 470 | 520 | B | 10 | 1 | | 70% | 100% | 130% | 150% | 170% | 180% |
| FIM4 | GBM2 | 470 | 520 | D | 50 | 5 | | | 20% | | | | |
| FIM4 | GBM7 | 470 | 520 | B | 30 | 5 | | 315% | 420% | | | | |
| FIM4 | GBM8 | 470 | 520 | B | 30 | 5 | | | 20% | | | | |
| FIM4 | GBM11 | 470 | 520 | I | 10 | 1 | | | 40% | | | | |
| FIM4 | GBM5 | 470 | 520 | B | 10 | 1 | | 70% | 90% | 100% | | | |
| FIM4 | GBM17 | 470 | 520 | B | 10 | 1 | | | 15% | | | | |
| FIM4 | GBM20 | 470 | 520 | B | 10 | 1 | | 135% | 160% | 170% | | | |
| FIM4 | GBM4 | 470 | 520 | B | 10 | 1 | | 230% | 280% | | | | |
| FIM4 | GBM18 | 470 | 520 | B | 10 | 1 | | 120% | 140% | 160% | | | |
| FIM5 | GBM16 | 470 | 520 | B | 10 | 1 | | 435% | 565% | 670% | 700% | | |
| FIM5 | GBM14 | 470 | 520 | B | 10 | 1 | | 330% | 425% | 530% | 560% | | |
| FIM5 | GBM15 | 470 | 520 | B | 10 | 1 | | | 185% | | | | |
| FIM5 | GBM13 | 470 | 520 | B | 10 | 1 | | 20% | 40% | 70% | 95% | | |
| FIM5 | GBM4 | 470 | 520 | B | 10 | 1 | | 350% | 495% | 625% | 685% | 720% | |
| FIM6 | GBM16 | 470 | 520 | D | 50 | 5 | | 120% | 155% | 175% | 190% | | |
| FIM6 | GBM15 | 470 | 520 | D | 50 | 5 | | | 20% | | | | |
| FIM7 | GBM15 | 470 | 520 | B | 10 | 10 | | | 75% | | | | |
| FIM7 | GBM15 | 470 | 520 | B | 20 | 10 | | | 160% | | | | |
| FIM7 | GBM15 | 470 | 520 | B | 50 | 10 | | 240% | 270% | | | | |
| FIM7 | GBM16 | 470 | 520 | B | 10 | 1 | | 400% | 520% | 645% | 710% | | |
| FIM7 | GBM16 | 470 | 520 | B | 50 | 5 | | 410% | 530% | 660% | 725% | | |
| FIM7 | GBM7 | 470 | 520 | B | 10 | 1 | | 110% | 140% | | | | |
| FIM7 | GBM7 | 470 | 520 | B | 30 | 5 | | 100% | 130% | 165% | 200% | | |
| FIM7 | GBM7 | 470 | 520 | B | 100 | 1 | | 40% | 75% | 100% | | | |
| FIM7 | GBM16 | 470 | 520 | C | 50 | 5 | | | 30% | | | | |
| FIM7 | GBM15 | 470 | 520 | C | 50 | 5 | | | 5% | | | | |
| FIM7 | GBM13 | 470 | 520 | C | 50 | 5 | | | 45% | | | | |
| FIM8 | GBM16 | 380 | 420 | B | 10 | 1 | | | 30% | | | | |
| FIM8 | GBM14 | 380 | 420 | B | 10 | 1 | | | 5% | | | | |
| FIM8 | GBM15 | 380 | 420 | B | 10 | 1 | | | −5% | | | | |
| FIM8 | GBM13 | 380 | 420 | B | 10 | 1 | | | 40% | | | | |
| FIM8 | GBM4 | 380 | 420 | B | 10 | 1 | | | 95% | | | | |
| FIM8 | GBM20 | 380 | 420 | B | 10 | 1 | | | 45% | | | | |
| FIM8 | GBM18 | 380 | 420 | B | 10 | 1 | | | 5% | | | | |
| FIM8 | GBM5 | 380 | 420 | B | 10 | 1 | | | 30% | | | | |
| FIM8 | GBM3 | 380 | 420 | B | 10 | 1 | | | 40% | | | | |
| FIM9 | GBM16 | 430 | 550 | B | 10 | 1 | | | 10% | | | | |
| FIM9 | GBM14 | 430 | 550 | B | 10 | 1 | | | 5% | | | | |
| FIM9 | GBM15 | 430 | 550 | B | 10 | 1 | | | 5% | | | | |
| FIM9 | GBM13 | 430 | 550 | B | 10 | 1 | | | 5% | | | | |
| FIM9 | GBM4 | 430 | 550 | B | 10 | 1 | | | 20% | | | | |
| FIM9 | GBM20 | 430 | 550 | B | 10 | 1 | | | 5% | | | | |
| FIM9 | GBM7 | 430 | 550 | B | 10 | 1 | | | 20% | | | | |
| FIM10 | GBM16 | 560 | 610 | B | 10 | 1 | | | 40% | | | | |
| FIM10 | GBM14 | 560 | 610 | B | 10 | 1 | | 10% | 20% | | | | |
| FIM10 | GBM15 | 560 | 610 | B | 10 | 1 | | | 70% | | | | |
| FIM10 | GBM13 | 560 | 610 | B | 10 | 1 | | | 5% | | | | |
| FIM10 | GBM4 | 560 | 610 | B | 10 | 1 | | | 5% | | | | |
| FIM10 | GBM3 | 560 | 610 | B | 10 | 1 | | | 5% | | | | |
| FIM10 | GBM15 | 560 | 610 | B | 30 | 1 | | | 85% | | | | |
| FIM10 | GBM15 | 560 | 610 | B | 50 | 1 | | | 105% | | | | |
| FIM10 | GBM15 | 560 | 610 | B | 100 | 1 | | 75% | 95% | 105% | | | |
| FIM10 | GBM15 | 560 | 610 | B | 200 | 1 | 50% | 70% | 95% | 110% | | | |
| FIM10 | GBM15 | 560 | 610 | B | 20 | 5 | | | 95% | | | | |
| FIM10 | GBM15 | 560 | 610 | B | 50 | 5 | | | 30% | | | | |
| FIM10 | GBM15 | 560 | 610 | D | 200 | 1 | | 20% | 30% | 40% | 50% | | |
| FIM10 | GBM15 | 560 | 610 | D | 5 | 1 | | | 5% | | | | |
| FIM10 | GBM15 | 560 | 610 | D | 50 | 5 | | | 10% | | | | |
| FIM10 | GBM15 | 560 | 610 | D | 100 | 1 | | | 20% | | | | |
| FIM10 | GBM8 | 560 | 610 | B | 30 | 1 | | | 20% | | | | |
| FIM10 | GBM7 | 560 | 610 | B | 30 | 1 | | | 65% | | | | |
| FIM11 | GBM16 | 470 | 520 | B | 10 | 1 | | | 150% | | | | |
| FIM11 | GBM14 | 470 | 520 | B | 10 | 1 | | | 140% | | | | |
| FIM11 | GBM15 | 470 | 520 | B | 10 | 1 | | | 5% | | | | |
| FIM11 | GBM13 | 470 | 520 | B | 10 | 1 | | 140% | 170% | 190% | 200% | | |
| FIM11 | GBM3 | 470 | 520 | B | 10 | 1 | | 90% | 105% | 110% | | | |
| FIM11 | GBM4 | 470 | 520 | B | 10 | 1 | | 300% | 320% | 350% | | | |
| FIM11 | GBM21 | 470 | 520 | D | 50 | 5 | | 30% | 35% | 40% | 45% | | |
| FIM11 | GBM13 | 470 | 520 | D | 50 | 5 | | 45% | 60% | 70% | 80% | | |
| FIM11 | GBM13 | 470 | 520 | B | 50 | 5 | | 90% | 120% | 180% | | | |
| FIM11 | GBM13 | 470 | 520 | B | 200 | 1 | | 15% | 30% | 60% | 80% | 90% | |
| FIM11 | GBM7 | 470 | 520 | B | 30 | 5 | | | 40% | | | | |
| FIM11 | GBM15 | 470 | 520 | B | 100 | 5 | | | 20% | | | | |
| FIM12 | GBM21 | 470 | 520 | B | 10 | 1 | | 20% | 30% | 45% | | | |
| FIM12 | GBM21 | 470 | 520 | B | 50 | 5 | | | 20% | | | | |
| FIM12 | GBM21 | 470 | 520 | B | 5 | 1 | | | 20% | | | | |
| FIM13 | GBM16 | 470 | 520 | B | 50 | 10 | | | 30% | | | | |
| FIM14 | GBM16 | 470 | 520 | B | 10 | 1 | | | 20% | | | | |
| FIM14 | GBM14 | 470 | 520 | B | 10 | 1 | | | 20% | 40% | | | |
| FIM14 | GBM15 | 470 | 520 | B | 10 | 1 | | | 10% | | | | |
| FIM14 | GBM13 | 470 | 520 | B | 10 | 1 | | | 20% | | | | |
| FIM15 | GBM15 | 450 | 660 | B | 10 | 1 | | | −30% | | | | |
| FIM15 | GBM15 | 450 | 660 | B | 1 | 1 | | | −10% | | | | |
| FIM15 | GBM15 | 450 | 660 | B | 50 | 1 | | | −25% | | | | |
| FIM15 | GBM15 | 450 | 660 | B | 50 | 5 | | | −35% | | | | |
| FIM15 | GBM15 | 450 | 660 | B | 50 | 10 | | | −35% | | | | |
| FIM15 | GBM15 | 450 | 660 | B | 100 | 10 | | | −25% | | | | |
| FIM16 | GBM5 | 515 | 550 | B | 10 | 1 | | 80% | 150% | 240% | 350% | 380% | 410% |
| FIM16 | GBM17 | 515 | 550 | B | 10 | 1 | | | 30% | | | | |
| FIM16 | GBM20 | 515 | 550 | B | 10 | 1 | | 150% | 240% | 350% | 410% | | |
| FIM16 | GBM4 | 515 | 550 | B | 10 | 1 | | 105% | 175% | 280% | 350% | 410% | 430% |
| FIM16 | GBM18 | 515 | 550 | B | 10 | 1 | | 80% | 160% | 230% | 320% | 370% | 420% |
| FIM16 | GBM13 | 515 | 550 | B | 10 | 1 | | 80% | 150% | | | | |
| FIM16 | GBM16 | 515 | 550 | B | 10 | 1 | | 150% | 250% | 350% | 410% | | |
| FIM16 | GBM14 | 515 | 550 | B | 10 | 1 | | | 95% | | | | |
| FIM16 | GBM15 | 515 | 550 | B | 10 | 1 | | | 40% | | | | |
| FIM16 | GBM16 | 515 | 550 | D | 50 | 5 | | | 90% | | | | |
| FIM16 | GBM15 | 515 | 550 | D | 50 | 5 | | | 40% | | | | |
| FIM16 | GBM3 | 515 | 550 | B | 10 | 1 | | 70% | 115% | 180% | 215% | 260% | 285% |
| FIM16 | GBM16 | 515 | 550 | C | 50 | 5 | | | 40% | | | | |
| FIM16 | GBM15 | 515 | 550 | C | 50 | 5 | | | 30% | | | | |
| FIM16 | GBM13 | 515 | 550 | C | 50 | 5 | | | 40% | | | | |
| FIM17 | GBM6 | 470 | 520 | B | 10 | 1 | | 10% | 15% | 20% | 20% | 30% | |
| FIM18 | GBM14 | 500 | 580 | B | 10 | 20 | | | 90% | | | | |
| FIM18 | GBM16 | 500 | 580 | B | 10 | 5 | | | 30% | | | | |
| FIM18 | GBM14 | 500 | 580 | B | 20 | 10 | | | 90% | | | | |
| FIM18 | GBM13 | 500 | 580 | B | 20 | 10 | | | 20% | | | | |
| FIM18 | GBM15 | 500 | 580 | B | 20 | 10 | | | 25% | | | | |
| FIM18 | GBM12 | 500 | 580 | B | 5 | 5 | 125% | | | | | | |
| FIM19 | GBM9 | 450 | 580 | B | N/A | N/A | | | | | | | |
| FIM20 | GBM16 | 420 | 500 | B | 20 | 2 | | | −20% | | | | |
| FIM21 | GBM16 | 470 | 520 | B | 40 | 1 | | | 50% | | | | |
| FIM21 | GBM16 | 470 | 520 | B | 200 | 1 | | | 35% | | | | |
| FIM21 | GBM14 | 470 | 520 | B | 20 | 1 | | | 45% | | | | |
| FIM21 | GBM15 | 470 | 520 | B | 10 | 1 | | | 5% | | | | |
| FIM21 | GBM13 | 470 | 520 | B | 20 | 1 | | | 5% | | | | |
| FIM22 | GBM10 | 470 | 520 | B | 10 | 5 | 98% | 115% | 120% | | | | |
| FIM22 | GBM16 | 470 | 520 | B | 40 | 1 | | | 110% | | | | |
| FIM22 | GBM14 | 470 | 520 | B | 20 | 1 | | | 65% | | | | |
| FIM22 | GBM15 | 470 | 520 | B | 10 | 1 | | | 10% | | | | |
| FIM22 | GBM13 | 470 | 520 | B | 10 | 1 | | | 170% | | | | |
| FIM23 | GBM13 | 470 | 520 | B | 10 | 1 | | | 40% | | | | |
| FIM23 | GBM16 | 470 | 520 | B | 10 | 1 | | | 90% | | | | |
| FIM23 | GBM14 | 470 | 520 | B | 10 | 1 | | | 60% | | | | |
| FIM23 | GBM15 | 470 | 520 | B | 10 | 1 | | | 10% | | | | |
| FIM23 | GBM7 | 470 | 520 | B | 10 | 1 | | | 5% | | | | |
| FIM23 | GBM7 | 470 | 520 | B | 30 | 5 | | | 10% | | | | |
| FIM24 | GBM16 | 470 | 520 | B | 10 | 1 | | | 50% | | | | |
| FIM24 | GBM14 | 470 | 520 | B | 10 | 1 | | | 40% | | | | |
| FIM24 | GBM15 | 470 | 520 | B | 10 | 1 | | | 80% | | | | |
| FIM24 | GBM13 | 470 | 520 | B | 10 | 1 | | | 5% | | | | |
| FIM24 | GBM7 | 470 | 520 | B | 30 | 5 | | | 10% | | | | |
| FIM25 | GBM16 | 470 | 520 | B | 10 | 1 | | 50% | 100% | 125% | 150% | 160% | |
| FIM25 | GBM14 | 470 | 520 | B | 10 | 1 | | | 15% | | | | |
| FIM25 | GBM15 | 470 | 520 | B | 10 | 1 | | | 10% | | | | |
| FIM25 | GBM13 | 470 | 520 | B | 10 | 1 | 10% | 20% | 30% | | | | |
| FIM26 | GBM16 | 490 | 570 | B | 10 | 1 | | | −45% | | | | |
| FIM26 | GBM14 | 490 | 570 | B | 10 | 1 | | | −50% | | | | |
| FIM26 | GBM15 | 490 | 570 | B | 10 | 1 | | | −60% | | | | |
| FIM26 | GBM13 | 490 | 570 | B | 10 | 1 | | | −30% | | | | |
| FIM27 | GBM16 | 750 | 810 | B | 10 | 1 | | | −20% | | | | |
| FIM27 | GBM14 | 750 | 810 | B | 10 | 1 | | | −50% | | | | |
| FIM27 | GBM15 | 750 | 810 | B | 10 | 1 | | | −90% | | | | |
| FIM27 | GBM13 | 750 | 810 | B | 10 | 1 | | | −60% | | | | |
| FIM28 | GBM8 | 470 | 520 | B | 10 | 1 | | | 10% | | | | |
| FIM28 | GBM8 | 470 | 520 | B | 30 | 1 | | | 15% | | | | |
| FIM28 | GBM8 | 470 | 520 | B | 30 | 5 | | 20% | 30% | 40% | | | |
| FIM28 | GBM8 | 470 | 520 | B | 100 | 5 | | | 10% | | | | |
| FIM28 | GBM7 | 470 | 520 | B | 30 | 5 | | | 70% | | | | |
| FIM28 | GBM21 | 470 | 520 | B | 10 | 1 | | 35% | 60% | 80% | 100% | | |
| FIM28 | GBM21 | 470 | 520 | B | 5 | 1 | | 55% | 70% | 80% | 90% | 100% | |
| FIM28 | GBM21 | 470 | 520 | B | 30 | 1 | | 40% | 50% | 60% | 70% | 80% | |
| FIM28 | GBM21 | 470 | 520 | B | 10 | 1 | | 15% | 25% | 45% | 60% | | |
| FIM29 | GBM8 | 560 | 610 | B | 30 | 1 | | 5% | 10% | 15% | | | |
| FIM29 | GBM7 | 560 | 610 | B | 30 | 1 | | | 35% | | | | |
| FIM29 | GBM15 | 560 | 610 | B | 30 | 1 | | | 60% | | | | |
| FIM30 | GBM15 | 500 | 660 | B | 5 | 1 | | | −25% | | | | |
| FIM30 | GBM14 | 500 | 685 | A | 1 | 1 | | | −15% | | | | |
| FIM30 | GBM14 | 500 | 685 | B | 1 | 1 | | | −5% | | | | |
| FIM30 | GBM13 | 500 | 685 | A | 1 | 1 | | | −5% | | | | |
| FIM31 | GBM22 | 470 | 590 | A | 5 | 1 | | | 90% | | | | |
| FIM32 | GBM15 | 470 | 520 | B | 10 | 1 | 170% | 190% | 200% | 220% | | | |
| FIM32 | GBM15 | 470 | 520 | B | 50 | 0.5 | 140% | 195% | 240% | 265% | 310% | | |
| FIM32 | GBM15 | 470 | 520 | B | 50 | 5 | 110% | 155% | 185% | 205% | 225% | 235% | |
| FIM32 | GBM15 | 470 | 520 | B | 50 | 10 | 110% | 145% | 190% | | | | |
| FIM32 | GBM15 | 470 | 520 | B | 100 | 1 | 95% | 135% | 160% | 220% | | | |
| FIM32 | GBM15 | 470 | 520 | B | 200 | 2 | 85% | 115% | 145% | 200% | 215% | | |
| FIM32 | GBM15 | 470 | 520 | B | 200 | 5 | 50% | 85% | 125% | 175% | 190% | 200% | |
| FIM32 | GBM15 | 470 | 520 | B | 200 | 10 | 50% | 85% | 115% | 130% | 160% | 170% | |
| FIM33 | GBM15 | 470 | 520 | B | 100 | 1 | | | 30% | | | | |
| FIM33 | GBM16 | 470 | 520 | B | 100 | 1 | | 10% | 20% | 25% | 30% | 35% | |
| FIM33 | GBM2 | 470 | 520 | B | 50 | 1 | | | 5% | | | | |
| FIM33 | GBM7 | 470 | 520 | B | 30 | 1 | | | 20% | | | | |
| FIM33 | GBM21 | 470 | 520 | B | 50 | 5 | | 5% | 15% | 20% | 20% | | |
| FIM33 | GBM13 | 470 | 520 | B | 50 | 5 | | 5% | 10% | | | | |
| FIM33 | GBM30 | 470 | 520 | B | 50 | 1 | | | 30% | | | | |
| FIM34 | GBM15 | 470 | 520 | B | 10 | 1 | 145% | 185% | 220% | 240% | | | |
| FIM34 | GBM15 | 470 | 520 | B | 50 | 1 | 65% | 75% | 95% | 120% | | | |
| FIM34 | GBM15 | 470 | 520 | B | 50 | 5 | 45% | 65% | 90% | 110% | 120% | | |
| FIM34 | GBM15 | 470 | 520 | B | 100 | 1 | 35% | 55% | 80% | 120% | | | |
| FIM34 | GBM13 | 470 | 520 | B | 50 | 1 | 15% | 25% | 35% | 45% | | | |
| FIM34 | GBM7 | 470 | 520 | B | 30 | 1 | 210% | 360% | 450% | 535% | 560% | | |
| FIM34 | GBM30 | 470 | 520 | B | 40 | 1 | 65% | 90% | 120% | 140% | 150% | | |
| FIM34 | GBM34 | 470 | 530 | B | 5 | 5 | 10% | 15% | 20% | | | | |
| FIM34 | GBM37 | 470 | 520 | B | 10 | 1 | 60% | 70% | 85% | 95% | | | |
| FIM34 | GBM37 | 470 | 520 | B | 20 | 2 | 90% | 115% | | | | | |
| FIM35 | GBM29 | 470 | 520 | B | 50 | 1 | | | 60% | | | | |
| FIM35^{a} | GBM29^{a} | 470 | 520 | B | 50 | 1 | | | 40% | | | | |
| FIM36 | GBM8 | 470 | 520 | B | 30 | 1 | 5% | 10% | 15% | 20% | | | |
| FIM36 | GBM15 | 470 | 520 | B | 50 | 1 | 15% | 20% | 30% | 35% | | | |
| FIM36 | GBM30 | 470 | 520 | B | 50 | 2 | 35% | 50% | 55% | 60% | | | |
| FIM36 | GBM30 | 470 | 520 | B | 50 | 5 | 25% | 40% | 45% | 50% | | | |
| FIM36 | GBM37 | 470 | 520 | B | 20 | 5 | 40% | 45% | 50% | | | | |
| FIM39 | GBM7 | 470 | 520 | B | 30 | 1 | 95% | 115% | 120% | | | | |
| FIM40 | GBM15 | 470 | 520 | B | 50 | 1 | −5% | −10% | −15% | −20% | | | |
| FIM40 | GBM30 | 470 | 520 | B | 30 | 1 | −5% | −10% | −15% | −15% | | | |
| FIM41 | GBM15 | 470 | 520 | B | 50 | 1 | 35% | 43% | 47% | 55% | | | |
| FIM41 | GBM15 | 470 | 520 | B | 50 | 5 | 35% | 44% | 52% | 55% | | | |
| FIM41 | GBM30 | 470 | 520 | B | 50 | 1 | | 45% | 60% | 65% | | | |
| FIM41 | GBM30 | 470 | 520 | B | 50 | 0.5 | 37% | 45% | 50% | | | | |
| FIM42 | GBM15 | 450 | 660 | B | 50 | 5 | −2% | −4% | −9% | −14% | −20% | −23% | |
| FIM43 | GBM15 | 450 | 660 | B | 50 | 5 | −4% | −6% | −10% | −15% | −19% | −21% | |
| FIM43 | GBM30 | 450 | 660 | B | 50 | 5 | −7% | −12% | −18% | −20% | −20% | | |
| FIM44 | GBM15 | 450 | 620 | B | 5 | 1 | −3% | −6% | −11% | −14% | −19% | −24% | |
| FIM44 | GBM15 | 450 | 620 | B | 1 | 1 | −13% | −18% | −22% | −26% | −29% | −30% | |
| FIM45 | GBM30 | 450 | 620 | B | 5 | 1 | −6% | −12% | −15% | −18% | −21% | −25% | |
| FIM46 | GBM15 | 450 | 620 | B | 50 | 5 | −5% | −7% | −11% | −15% | −29% | | |
| FIM47 | GBM15 | 470 | 520 | B | 50 | 5 | 130% | 190% | 260% | 310% | 340% | 350% | |
| FIM48 | GBM15 | 450 | 660 | B | 50 | 5 | −10% | −17% | −18% | −22% | −25% | | |
| FIM49 | GBM30 | 450 | 660 | B | 50 | 5 | −12% | −17% | −25% | −25% | | | |
| FIM50 | GBM15 | 470 | 520 | B | 100 | 2 | 50% | 70% | 90% | | | | |
| FIM50 | GBM7 | 470 | 520 | B | 50 | 2 | 140% | 180% | 200% | | | | |
| FIM51 | GBM15 | 450 | 660 | B | 10 | 1 | −10% | −16% | −25% | −28% | | | |
| FIM53 | GBM30 | 450 | 660 | B | 10 | 1 | −8% | −11% | −13% | −17% | | | |
| FIM54 | GBM30 | 470 | 520 | B | 50 | 2 | 15% | 20% | 30% | 50% | | | |

^{a}Separate GBM and FIM components were mixed with 0.25 molar equivalents of streptavidin tetramer prior to formulation of assay.

TABLE 2

| | | | | Assay | Change in Emₘₐₓ upon addition of D-glucose (concentration listed below)/% | | | | | | |
|---|---|---|---|---|---|---|---|---|---|---|---|
| GBM | [GBM]/μM | Ex/nm | Emₘₐₓ/nm | Medium | 5 mM | 10 mM | 20 mM | 40 mM | 60 mM | 80 mM | 100 mM |
| GBM23 | 10 | 450 | 530 | B | 8% | 15% | 23% | | | | |
| GBM24 | 10 | 450 | 530 | B | 0% | 0% | 0% | | | | |
| GBM25 | 10 | 450 | 530 | B | 8% | 16% | 23% | | | | |
| GBM26 | 10 | 450 | 530 | B | 8% | 12% | 16% | | | | |
| GBM27 | 10 | 515 | 550 | B | 10% | 13% | 16% | 26% | | | |
| GBM28 | 10 | 450 | 670 | B | 0% | 0% | 0% | | | | |
| GBM35 | 0.1 | 470 | 530 | B | 21% | 30% | 39% | 45% | | | |
| GBM35 | 1 | 470 | 530 | B | 22% | 33% | 37% | 43% | | | |
| GBM35 | 5 | 470 | 530 | B | 18% | 25% | 35% | 40% | | | |
| GBM35 | 10 | 470 | 530 | B | 15% | 18% | 25% | 35% | | | |
| GBM33 | 0.1 | 470 | 530 | B | 10% | 10% | 10% | 10% | | | |
| GBM33 | 1 | 470 | 530 | B | 30% | 40% | 45% | 45% | | | |
| GBM33 | 5 | 470 | 530 | B | 60% | 85% | 120% | 145% | | | |
| GBM33 | 10 | 470 | 530 | B | 40% | 60% | 85% | 90% | | | |
| GBM36 | 10 | 470 | 530 | B | −15% | −25% | −30% | −35% | | | |
| GBM36 | 5 | 470 | 530 | B | −10% | −12% | −15% | −18% | | | |
| GBM36 | 1 | 470 | 530 | B | −15% | −15% | −10% | −10% | | | |
| GBM36 | 0.1 | 470 | 530 | B | 25% | 45% | 75% | 110% | 145% | 185% | 210% |
| GBM36 | 0.05 | 470 | 530 | B | 20% | 35% | 55% | 85% | 110% | 130% | 150% |
| GBM38 | 0.1 | 470 | 530 | B | 30% | 45% | 75% | 80% | | | |

TABLE 3

| | | Ex/ | Emₘₐₓ/ | Assay | [GBM]/ | [FIM]/ | Change in Emₘₐₓ for FIM at a particular D-glucose concentration/% | | | | | | |
|---|---|---|---|---|---|---|---|---|---|---|---|---|---|
| FIM | GBM | nm | nm | Medium | uM | uM | 5 mM | 10 mM | 20 mM | 40 mM | 60 mM | 80 mM | 100 mM |
| FIM10 | GBM40 | 560 | 610 | B | 5 | 10 | | | | −75% | | | |
| FIM10 | GBM42 | 560 | 610 | B | 20 | 5 | 65% | 85% | 105% | 110% | | | |
| FIM43 | GBM42 | 450 | 640 | B | 20 | 5 | 35% | 65% | 90% | 125% | | 145% | |
| FIM44 | GBM42 | 450 | 640 | B | 20 | 5 | 10% | 20% | 30% | 45% | | 55% | |
| FIM47 | GBM40 | 470 | 520 | B | 20 | 5 | 265% | 410% | 555% | 710% | | 860% | |
| FIM49 | GBM42 | 450 | 640 | B | 20 | 5 | 20% | 35% | 45% | 55% | | 60% | |
| FIM57 | GBM15 | 450 | 640 | B | 10 | 1 | | | −25% | −40% | | | |
| FIM57 | GBM15 | 450 | 640 | C | 10 | 1 | | | −22% | −25% | | | |
| FIM57 | GBM42 | 450 | 640 | B | 20 | 5 | 30% | 45% | 80% | 120% | | 150% | |
| FIM57 | GBM42 | 450 | 640 | C | 20 | 5 | 15% | 20% | 30% | 35% | | 45% | 50% |
| FIM58 | GBM15 | 450 | 640 | B | 50 | 5 | −5% | −10% | −15% | −20% | | −25% | |
| FIM59 | GBM15 | 450 | 640 | B | 50 | 5 | −5% | −10% | −20% | −25% | | −30% | |
| FIM59 | GBM42 | 450 | 640 | B | 20 | 5 | 115% | 170% | 135% | 300% | | 380% | 420% |
| FIM60 | GBM7 | 470 | 520 | B | 50 | 5 | 220% | | 310% | 400% | | | |
| FIM60 | GBM15 | 470 | 520 | B | 50 | 5 | 80% | 130% | 200% | 220% | | | |
| FIM60 | GBM40 | 470 | 520 | B | 20 | 5 | 250% | | 450% | | | 600% | |
| FIM60 | GBM40 | 470 | 520 | C | 20 | 5 | 250% | | 430% | | | 500% | |
| FIM60 | GBM30 | 470 | 520 | B | 50 | 1 | 120% | 170% | 200% | 220% | | | |
| FIM60 | GBM43 | 450 | 520 | B | 40 | 5 | 80% | 125% | 165% | 200% | | 230% | |
| FIM61 | GBM30 | 450 | 520 | B | 100 | 1 | 85% | 120% | 160% | 190% | | | |
| FIM61 | GBM30 | 450 | 520 | C | 100 | 1 | 70% | 145% | 190% | 210% | | | |
| FIM61 | GBM43 | 450 | 520 | B | 40 | 5 | 80% | 125% | 170% | 200% | | | |
| FIM61 | GBM43 | 450 | 520 | C | 50 | 50 | 80% | 120% | 165% | 195% | | | |
| FIM61 | GBM41 | 470 | 520 | B | 50 | 1 | 90% | 110% | 120% | | | | |
| FIM62 | GBM43 | 450 | 520 | B | 50 | 1 | 100% | 145% | 200% | 230% | | | |
| FIM63 | GBM43 | 450 | 520 | B | 50 | 5 | 130% | 180% | 235% | 270% | | | |
| FIM64 | GBM42 | 450 | 640 | B | 20 | 5 | 115% | 150% | 170% | | | | |
| FIM65 | GBM42 | 560 | 610 | B | 25 | 5 | 180% | 230% | 285% | 320% | | | |
| FIM65 | GBM15 | 560 | 610 | B | 50 | 5 | 20% | 30% | 40% | 55% | | 60% | |
| FIM66 | GBM15 | 470 | 520 | B | 50 | 5 | 250% | 255% | 260% | | | | |
| FIM68 | GBM30 | 560 | 610 | B | 50 | 5 | 30% | 40% | 45% | 50% | | | |
| FIM68 | GBM42 | 560 | 610 | B | 20 | 5 | 285% | 380% | 440% | 480% | | | |
| FIM68 | GBM44 | 560 | 610 | B | 30 | 5 | 25% | 35% | 55% | 75% | | 110% | 120% |
| FIM68 | GBM44 | 560 | 610 | B | 15 | 2 | 115% | 140% | 160% | 175% | | | |

TABLE 4

| Medium | Code |
|---|---|
| 10 mM PB | A |
| 10 mM PBS | B |
| Human serum | C |
| Filtered human serum (30 kDa MWCO) | D |
| 1:1 Ethylene Glycol/Water | E |
| 10% wt Pluronic F-127 in 10 mM PBS | F |
| 10% wt Dextran (70 kDa) in 10 mM PBS | G |
| 10% wt Polyacrylamide (150 kDa) in 10 mM PBS | H |
| 30% wt Polyacrylamide (150 kDa) in 10 mM PBS | I |
| 10 mM PBS + 1.25 mM 40 kDa 8-arm PEG azide | J |
| 10 mM PBS + 15 mM 3.4 kDa bis-NHS PEG | K |

### Fluorescent Hydroqel Phase Glucose Responsive Assays

### Hydrogel Formation Procedures

### Hydrogel A—HEMA/TEGDMA

Hydroxyethylmethacrylate (2.85 g), TEGDMA (0.15 g), VAZO-44 (0.12 mg), ethylene glycol (1.06 g) and water (1.33 g) were added to a glass vial and vortexed/sonicated until a homogeneous solution. Concentrated aliquots (<50 μL) of the appropriate GBM and FIM from pre-made stock solutions were added to give the required final concentrations of GBM and FIM (i.e. 50 μL of GBM stock solution at 1 mM gives a final GBM concentration of 10 μM in the pre-gel solution). A 50 μL aliquot of this GBM and FIM containing pre-gel solution was pipetted into each well of a transparent flat-bottomed polystyrene 96 well plate. 100 μL of n-butanol was layered on top of the pre-gel solution in each well plate and each well plate covered with a loose transparent well plate lid. The well plate was then placed in a nitrogen flushed desiccator, which was then placed into a pre-heated oven at 45° C. for 4 hours. The well plates were then removed from the oven, allowed to cool to RT and the top solution removed by decanting onto an absorbent material. The hydrogels were then washed and swelled by submerging in one litre of pure water or 10 mM PBS for 24 hours. Each well within the well plate containing hydrogel bound assay was then washed with D-glucose (50 mM) and then 10 mM PBS multiple times until the observed fluorescence emission stabilised to give a baseline emission value in 10 mM PBS. These hydrogels were then used for determining assay performance and reversibility studies.

### Hydrogel B—HEMA/PEGDA

Hydroxyethylmethacrylate (1.95 g), PEGDA (0.57 g), VAZO-44 (0.04 mg), ethylene glycol (1.06 g) and 200 mM methyl-p-D-glucopyranoside (aqueous, 1.39 g) were added to a glass vial and vortexed/sonicated until a homogeneous solution. Concentrated aliquots (<50 μL) of the appropriate GBM and FIM from pre-made stock solutions were added to give the required final concentrations of GBM and FIM. A 50 μL aliquot of this GBM and FIM containing pre-gel solution was pipetted into each well of a transparent flat-bottomed polystyrene 96 well plate. 100 μL of n-butanol was layered on top of the pre-gel solution in each well plate and each well plate covered with a loose transparent well plate lid. The well plate was then placed in a nitrogen flushed desiccator, which was then placed into a pre-heated oven at 45° C. for 4 hours. The well plates were then removed from the oven, allowed to cool to RT and the top solution removed by decanting onto an absorbent material. The hydrogels were then washed and swelled by submerging in one litre of pure water or 10 mM PBS for 24 hours. Each well within the well plate containing hydrogel bound assay was then washed with D-glucose (50 mM) and then 10 mM PBS multiple times until the observed fluorescence emission stabilised to give a baseline emission value in 10 mM PBS. These hydrogels were then used for determining assay performance and reversibility studies.

Hydrogel C—Polyacrylamide/bis-acrylamide

Acrylamide (0.95 g, 30% wt in water), N,N′-Methylenebisacrylamide (0.05 g) and water (1 g) were added to a glass vial and the solution sparged with nitrogen gas for 5 minutes. Concentrated aliquots (<50 μL) of the appropriate GBM and FIM from pre-made stock solutions were added to give the required final concentrations of GBM and FIM. Added TMEDA (18 μL, 10% wt in water) and ammonium persulfate (18 μL, 10% wt in water) and vortexed to give a homogeneous solution. Immediately after this, a 50 μL aliquot of this GBM and FIM containing pre-gel solution was pipetted into each well of a transparent flat-bottomed polystyrene 96 well plate. 20 μL of n-butanol was layered on top of the pre-gel solution in each well plate and each well plate covered with a loose transparent well plate lid. The well plate was then placed in a nitrogen flushed desiccator for 30 minutes. The well plates were then removed from the oven and the top solution removed by decanting onto an absorbent material. The hydrogels were then washed and swelled by submerging in one litre of pure water or 10 mM PBS for 24 hours. Each well within the well plate containing hydrogel bound assay was then washed with D-glucose (50 mM) and then 10 mM PBS multiple times until the observed fluorescence emission stabilised to give a baseline emission value in 10 mM PBS. These hydrogels were then used for determining assay performance and reversibility studies.

Hydrogel D—Azido PEG star polymer

A solution of 8-arm PEG star polymer (20 mg, 40 kDa, 7-amine/1-azide) in water (0.2 mL) and a solution of bis-N-hydroxysuccinimidyl ester PEG crosslinker (20 mg, 3.4 kDa) in water (0.2 mL) were combined and vortexed briefly to give a homogenous solution. Immediately after this, a 3 μL aliquot of this solution was pipetted into each well of a transparent flat-bottomed polystyrene 96 well plate and left to crosslink for 2 hours. After this, the formed hydrogels in the wells were quenched with a solution of ethanolamine (30% wt in water) for 3 hours. The ethanolamine solution was then removed by decanting the well plate onto an absorbent material and the hydrogels were then soaked with pure water for 30 minutes, and this process repeated 3 more times. A 50 μL solution of GBM and FIM at the required concentration, which also contained 5 molar equivalents of sodium ascorbate (relative to the GBM concentration), was added to each well and allowed to be absorbed into the hydrogel over 30 minutes. A solution of CuSO₄·5H₂O (1 molar equivalent relative to GBM concentration) was then added to each well and the reaction left overnight at RT. After this, the reaction solution was removed by decanting the well plate onto an absorbent material and the hydrogels were then soaked with pure water for 30 minutes, and this process repeated 3 more times. Each well within the well plate containing hydrogel bound assay was then washed with D-glucose (50 mM) and then 10 mM PBS multiple times until the observed fluorescence emission stabilised to give a baseline emission value in 10 mM PBS. These hydrogels were then used for determining assay performance and reversibility studies.

Hydrogel E—Amino PEG star polymer

A solution of 8-arm PEG star polymer (20 mg, 40 kDa, 8-amine) in water (0.2 mL) and a solution of bis-N-hydroxysuccinimidyl ester PEG crosslinker (20 mg, 3.4 kDa) in water (0.2 mL) were combined and vortexed briefly to give a homogenous solution. Immediately after this, a 3 μL aliquot of this solution was pipetted onto a transparent polystyrene surface and left to crosslink for 2 hours. After this, the formed hydrogels were transferred to a vial and quenched with an aqueous solution of ethylene diamine (20 mL, 1:2 v/v ethylene diamine/water) for 16 hours. The ethylene diamine solution was then removed by pipette, the gels washed with 10 mM PBS (20 mL) 3 times and then swelled in 10 mM PBS for 16 hours. The swelled gels were then frozen with liquid nitrogen or allowed to dry out into thin discs, and these discs adhered with cyanoacrylate adhesive to either the walls of transparent polystyrene cuvettes or bottom of well plates. The adhesive was allowed to cure for 10 minutes and the gels swelled again with 10 mM PBS for 16 hours. The PBS solution was removed and the gels soaked in an aqueous solution (pH 8, adjusted with NaHCO₂) of GBM (and FIM if required) at the required concentration, which also contained EDC·HCl (2 mM) and HOBt (2 mM), for 2 hours. The reaction solution was removed by pipette and the gels washed with D-glucose (50 mM) and then 10 mM PBS multiple times until the observed fluorescence emission stabilised to give a baseline emission value in 10 mM PBS. These hydrogels were then used for determining assay performance and reversibility studies.

Hydrogel F—HEMA/acrylic acid/PEGDA

Hydroxyethylmethacrylate (500 mg), Acrylic acid (50 mg) and PEGDA-575 (150 mg) were dissolved in pure water (400 mg). APS (1 mol %) and TMEDA (1 drop) were added and solution vortexed briefly to give a homogeneous solution. A 50 μL aliquot of this solution was then pipetted into each well of a transparent flat-bottomed polystyrene 96 well plate. The well plate was then covered and the hydrogels left to cure for 4 hours. The hydrogels were then soaked in an aqueous solution (pH 8, adjusted with NaHCO₃) of ethylene diamine (1:9, ethylene diamine/water), which also contained EDC·HCl (100 mM) and N-hydroxysuccinimide (100 mM), for 16 hours. After this, the gels were washed with pure water 3 times and then soaked in pure water for 16 hours. The gels were then soaked in an aqueous solution (pH 8, adjusted with NaHCO₃) of GBM and FIM at the required concentration, which also contained EDC·HCl (2 mM) and HOBt (2 mM), for 2 hours. The reaction solution was removed by pipette and the gels swelled with 10 mM PBS for 16 hours. The gels were then washed with D-glucose (50 mM in 10 mM PBS) and 10 mM PBS multiple times until the observed fluorescence emission stabilised to give a baseline emission value in 10 mM PBS. These hydrogels were then used for determining assay performance and reversibility studies.

Hydrogel G—Amino Dextran/PEG/PEG3-azide

GBM at the required concentration was added to a 0.2 mL aqueous solution of EDC·HCl (100 mM) and N-hydroxysuccinimide (100 mM) and left at room temperature for 10 minutes. This solution was then added to amino dextran (25 mg, 70 kDa, -29 amines per dextran) and left at room temperature for 10 minutes. This solution was then combined with an aqueous solution of bis-N-hydroxysuccinimidyl ester PEG crosslinker (25 mg, 2 kDa) in water (0.2 mL), then vortexed briefly to give a homogenous solution. Immediately after this, 5 μL aliquots of the gel mixture were pipetted into each well of a transparent flat-bottomed polystyrene 96 well plate. The well plate was covered and the hydrogels left to cure for 2 hours. The hydrogels were then quenched with a solution of azido-PEG3-amine (50 mM) for 4 hours, then swelled in 10 mM PBS for 16 hours. The gels were then soaked with a solution of FIM, at the appropriate concentration, for 2 minutes before being washed with 10 mM PBS 3 times. The gels were then washed with D-glucose (50 mM in 10 mM PBS) and 10 mM PBS multiple times until the observed fluorescence emission stabilised to give a baseline emission value in 10 mM PBS. These hydrogels were then used for determining assay performance and reversibility studies.

Hydrogel H—Amino azide Dextran/PEG

GBM at the required concentration was added to a 0.2 mL aqueous solution of EDC·HCl (100 mM) and N-hydroxysuccinimide (100 mM) and left at room temperature for 10 minutes. This solution was then added to amino dextran (25 mg, 70 kDa, -29 amines per dextran) and left at room temperature for 10 minutes. Azidoacetic acid NHS ester (1 mM final concentration) in acetonitrile was added and left for 10 minutes. This dextran solution was then combined with an aqueous solution of bis-N-hydroxysuccinimidyl ester PEG crosslinker (25 mg, 2 kDa) in water (0.2 mL), then vortexed briefly to give a homogenous solution. Immediately after this, 5 μL aliquots of the gel mixture were pipetted into each well of a transparent flat-bottomed polystyrene 96 well plate. The well plate was covered and the hydrogels left to cure for 2 hours, after which they were swelled in 10 mM PBS for 16 hours. The gels were then soaked with a solution of FIM, at the appropriate concentration, for 2 minutes before being washed with 10 mM PBS 3 times. The gels were then washed with D-glucose (50 mM in 10 mM PBS) and 10 mM PBS multiple times until the observed fluorescence emission stabilised to give a baseline emission value in 10 mM PBS. These hydrogels were then used for determining assay performance and reversibility studies.

### Results of Glucose Responsive Assay

Results for glucose responsive fluorescence emission for different combinations of glucose binding molecules (GBM) and fluorescent inhibitor molecules (FIM) at various concentrations and in various hydrogel media were determined. All hydrogel assays were performed in the presence of 10 mM PBS. The results are shown in Table 5 below. All results obtained at pH 7.4 and 310 K unless otherwise stated.

Results for glucose responsive fluorescence emission for different combinations of glucose binding molecules (GBM) and fluorescent inhibitor molecules (FIM) at various concentrations and in various hydrogel media were determined. All hydrogel assays were performed in the presence of 10 mM PBS. The results are shown in Table 6 below. All results obtained at pH 7.4 and 310 K unless otherwise stated.

FIG. 4 shows an example cycling of hydrogel A containing GBM2 (10 μM) and FIM4 (1 μM) pre-incorporated into the hydrogel. Cycling between pure water and 20 mM D-glucose (in 10 mM PBS) shows reversibility of hydrogel. Intensity values for emission obtained at 310 K and pH 7.4.

FIG. 5 shows an example cycling of hydrogel D containing GBM1 (50 μM) and FIM1 (5 μM) pre-incorporated into the hydrogel. Cycling between 10 mM PBS and 20 mM D-glucose (in 10 mM PBS) shows reversibility of hydrogel. Intensity values for emission obtained at 310 K and pH 7.4. Two separate hydrogels are shown as blue and orange data.

FIG. 6 shows an example cycling of hydrogel E containing GBM36 (2 μM) pre-incorporated into the hydrogel. Cycling between 10 mM PBS and 20 mM D-glucose (in 10 mM PBS) shows reversibility of hydrogel. Intensity values for emission obtained at 298 K and pH 7.4

FIG. 7 shows an example cycling of hydrogel F containing FIM41 (5 μM) and GBM15 (50 μM) incorporated into the hydrogel. Cycling between 10 mM PBS, 20 mM D-glucose (in 10 mM PBS) and 40 mM D-glucose (in 10 mM PBS) shows reversibility of hydrogel. Intensity values for emission obtained at 298 K and pH 7.4.

FIG. 8 shows an example cycling of FIM61 (1 μM) and GBM30 (100 uM) while encapsulated within a regenerated cellulose membrane (MWCO 10 kDa). Cycling between 10 mM PBS and 20 mM D-glucose (in 10 mM PBS) shows reversibility of the assay while encapsulated within the membrane. The final step wise increases in emission pertain to the assay being exposed to 5, 10 and 20 mM D-glucose. Intensity values for emission obtained at 310 K and pH 7.4.

Glucose responsive fluorescence assay comprised of FIM61 (1 uM) and GBM30 (100 uM) was encapsulated within a regenerated cellulose membrane (MWCO kDa). The assay was equilibrated over 24 hours in 10 mM PBS (pH 7.4). The membrane bound assay was then equilibrated in 20 mM D-glucose (in 10 mM PBS) and the fluorescence emission recorded on a Clariostar plus plate reader (pre-equilibrated to 37° C.) using excitation wavelength 480 nm and monitoring emission wavelength of 520 nm. The membrane bound assay was then equilibrated back to baseline 0 mM D-glucose emission values with 10 mM PBS (no glucose). The membrane bound assay was then re-equilibrated to 20 mM D-glucose emission values, and this cycling between 0 mM and 20 mM glucose emission values repeated to demonstrate reversibility of the membrane bound glucose responsive assay. At the end of the cycling experiments, a stepwise equilibration of 5, 10 and 20 mM glucose was demonstrated to show the sensitivity of the membrane bound assay over this glucose concentration range.

TABLE 5

| | | Ex/ | Emₘₐₓ/ | | [GBM]/ | [FIM]/ | Change in Emₘₐₓ for FIM at a particular D-glucose concentration/% | | | | | | |
|---|---|---|---|---|---|---|---|---|---|---|---|---|---|
| FIM | GBM | nm | nm | Hydrogel | uM | uM | 5 mM | 10 mM | 20 mM | 50 mM | 60 mM | 80 mM | 100 mM |
| FIM1 | GBM1 | 470 | 530 | D | 10 | 1 | | | 10% | 20% | | | |
| FIM4 | GBM2 | 470 | 530 | A | 10 | 1 | 25% | 40% | 50% | 60% | | | |
| FIM4 | GBM2 | 470 | 530 | B | 10 | 1 | | 55% | 75% | 95% | | | |
| FIM4 | GBM2 | 470 | 530 | C | 10 | 1 | | | 45% | | | | |
| FIM16 | GBM2 | 515 | 550 | A | 10 | 1 | | | 45% | | | | |
| FIM16 | GBM2 | 515 | 550 | C | 10 | 1 | | | 50% | | | | |
| FIM17 | GBM39 | 470 | 530 | D | 50 | 2 | | | 70% | 175% | 215% | 295% | 380% |
| FIM21 | GBM2 | 470 | 530 | A | 10 | 1 | | | 0% | | | | |
| FIM22 | GBM2 | 470 | 530 | A | 10 | 1 | | | 10% | | | | |
| FIM22 | GBM2 | 470 | 530 | C | 10 | 1 | | | 15% | | | | |
| FIM23 | GBM2 | 470 | 530 | A | 10 | 1 | | | 10% | | | | |
| FIM23 | GBM2 | 470 | 530 | C | 10 | 1 | | | 15% | | | | |
| FIM24 | GBM2 | 470 | 530 | A | 10 | 1 | | | 8% | | | | |
| FIM25 | GBM2 | 470 | 530 | A | 10 | 1 | | | 12% | | | | |
| FIM47 | GBM39 | 470 | 530 | D | 50 | 2 | | | 40% | 100% | 120% | 160% | 190% |
| — | GBM36 | 470 | 530 | E | 2 | — | | | 60% | 140% | 160% | 200% | 250% |

TABLE 6

| | | Ex/ | Emₘₐₓ/ | | [GBM]/ | [FIM]/ | Change in Emₘₐₓ for FIM at a particular D-glucose concentration/% | | | | | | |
|---|---|---|---|---|---|---|---|---|---|---|---|---|---|
| FIM | GBM | nm | nm | Hydrogel | uM | uM | 5 mM | 10 mM | 20 mM | 40 mM | 60 mM | 80 mM | 100 mM |
| FIM17 | GBM30 | 480 | 520 | H | 100 | 10 | | | 120% | | | | |
| FIM41 | GBM15 | 470 | 530 | F | 50 | 5 | | | 140% | 200% | | | |
| FIM60 | GBM15 | 480 | 520 | G | 50 | 50 | | | 50% | | | | |
| FIM60 | GBM30 | 480 | 520 | H | 100 | 10 | | | 40% | | | | |
| FIM65 | GBMXX3 | 590 | 620 | G | 50 | 50 | | | 90% | | | | |
| FIM66 | GBM30 | 480 | 520 | H | 100 | 10 | | | 140% | | | | |
| FIM67 | GBM30 | 480 | 520 | H | 100 | 10 | | | 90% | | | | |

Results from Fluorescence Lifetime Glucose Responsive Assays

Table 7 shows changes in fluorescence lifetime for assay containing FIM15 and GBM15 at various concentrations of D-glucose. Measurements obtained at 298 K and pH 7.4 in 10 mM PBS medium.

TABLE 7

| [FIM]/ | [GBM]/ | [D-glucose]/ | Average τ/ | Δτ/ | |
|---|---|---|---|---|---|
| μM | μM | mM | ns | ns | % Change τ |
| 5 | 0 | 0 | 438 | — | — |
| 5 | 50 | 0 | 515 | 0 | 0% |
| 5 | 50 | 20 | 456 | −59 | −11% |
| 5 | 50 | 40 | 432 | −83 | −16% |

FIG. 9 is a graphical representation of data in Table 7, showing changes in fluorescence lifetime for assay containing FIM15 (5 μM) and GBM15 (50 μM) at various concentrations of D-glucose. Measurements obtained at 298 K and pH 7.4.

Table 8 shows changes in fluorescence lifetime for assays containing different combinations of GBM and FIM at various concentrations of D-glucose. Some FIM exhibited bi-exponential decays for lifetime, thus yielding two fluorescence lifetime components (i.e. T1 and T2). Measurements obtained using excitation wavelength of 450 nm at 298 K and pH 7.4 in 10 mM PBS medium.

TABLE 8

| | | | | Lifetime | | Lifetime components | | Fluorescence lifetime components (τx) for FIM and GBM | | | | | |
|---|---|---|---|---|---|---|---|---|---|---|---|---|---|
| | | | | components (τx) of | | (τx) of FIM after GBM | | at a particular glucose concentration/ns | | | | | |
| | | [GBM]/ | [FIM]/ | FIM only/ns | | addition/ns | | τ1 | | | τ2 | | |
| FIM | GBM | uM | uM | τ1 | τ2 | τ1 | τ2 | 5 mM | 20 mM | 40 mM | 5 mM | 20 mM | 40 mM |
| FIM10 | GBM40 | 5 | 10 | 575 | | 620 | | | 580 | | | | |
| FIM43 | GBM30 | 10 | 5 | 290 | | 355 | | | 315 | | | | |
| FIM45 | GBM30 | 5 | 1 | 550 | | 625 | | 575 | 550 | | | | |
| FIM53 | GBM30 | 10 | 5 | 250 | | 295 | | | | 265 | | | |
| FIM55 | GBM15 | 50 | 5 | 1010 | 3080 | 1300 | 2720 | 1520 | 1220 | 1180 | 3420 | 3330 | 3340 |
| FIM56 | GBM15 | 20 | 5 | 520 | 3620 | 650 | 2500 | 695 | 660 | 625 | 3040 | 3210 | 3260 |
| FIM57 | GBM15 | 10 | 5 | 550 | | 650 | | 580 | 560 | | | | |
| FIM59 | GBM15 | 40 | 10 | 500 | | 620 | | | | 515 | | | |
| FIM64 | GBM15 | 50 | 5 | 1095 | 2973 | 1110 | 1660 | 1095 | 1070 | 1100 | 1850 | 2230 | 2270 |

Results from Affinity Electrochemistry—Cyclic Voltammetry

Table 9 shows changes in applied potential (V) and current response (A) upon varying concentration of D-glucose during cyclic voltammetry for assays comprised of GBM15 and different FIM compounds at various concentrations. Changes in applied potential and current response were monitored using the reduction process observed during the cyclic voltammogram. Measurements obtained at 298 K and pH 7.4 in 10 mM PBS medium.

TABLE 9

| | | | | | Current |
|---|---|---|---|---|---|
| [D-glucose]/ | | [FIM]/ | [GBM]/ | Applied | response |
| mM | FIM | mM | mM | potential/V | (×10⁻⁶)/A |
| 0 | FIM38 | 0.5 | 0 | 0.327 | −4.25 |
| 0 | FIM38 | 0.5 | 1 | 0.280 | −3.1 |
| 1 | FIM38 | 0.5 | 1 | 0.285 | −3.75 |
| 2 | FIM38 | 0.5 | 1 | 0.300 | −4.25 |
| 3 | FIM38 | 0.5 | 1 | 0.306 | −4.4 |
| 4 | FIM38 | 0.5 | 1 | 0.310 | −4.5 |
| 5 | FIM38 | 0.5 | 1 | 0.310 | −4.5 |
| 1 | FIM52 | 0.5 | 1 | −0.287 | −9.11 |
| 2 | FIM52 | 0.5 | 1 | −0.258 | −9.52 |
| 10 | FIM52 | 0.5 | 1 | −0.239 | −10.0 |
| 20 | FIM52 | 0.5 | 1 | −0.231 | −11.1 |
| 50 | FIM52 | 0.5 | 1 | −0.231 | −11.4 |

FIG. 10 shows an example glucose responsive cyclic voltammetry for assay comprised of GBM15 (1 mM) and FIM38 (0.5 mM) in 10 mM PBS. Addition of D-glucose to assay shows a shift in both applied potential (V) and current response (A) for the redox active assay.

1. A saccharide sensing system, the sensing system includes a saccharide-binding compound having a saccharide-binding cavity for binding a target saccharide and a detectable reporter for providing a detectable signal, wherein the saccharide-binding compound is a compound of formula (I): 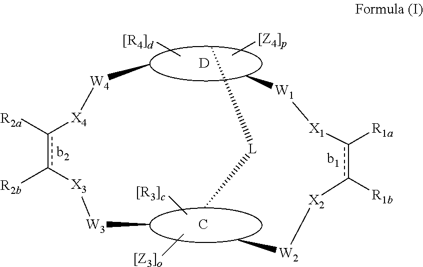
Wherein:
bonds b₁ and b₂ are independently selected from a single bond or double bond;
R₁ₐ, R_{1b}, R₂ₐ and R_{2b} are independently selected from hydrogen, carbonyl, (1-8C)alkyl, (3-10C)cycloalkyl, aryl, heteroaryl and heterocyclyl, each of which, other than hydrogen and carbonyl, is optionally substituted by one or more substituent groups selected from (1-4C)alkyl, halo, (1-4C)haloalkyl, (1-4C)haloalkoxy, (1-4C)alkoxy, (1-4C)alkylamino, amino, cyano, hydroxyl, carboxy, carbamoyl, sulfamoyl, mercapto and a hydrophilic substituent group;
or R₁ₐ and R_{1b} are linked so as to form a group of the formula: 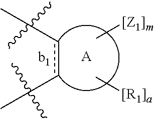
and/or R₂ₐ and R_{2b} are linked so as to form a group of the formula: 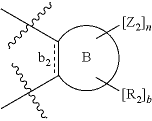
wherein: [symbol]
denotes the point of attachment;
bonds b₁ and b₂ are as described above;
Rings A and B are independently selected from aryl, heteroaryl heterocyclyl, cycloalkyl and cycloalkenyl;
R₁ and R₂ are independently selected from (1-6C)alkyl, halo, (1-4C)haloalkyl, (1-4C)haloalkoxy, (1-6C)alkoxy, (1-4C)alkylamino, amino, cyano, hydroxyl, carboxy, carbamoyl, sulfamoyl and mercapto;
a and b are integers independently selected from 0 to 2;
m and n are integers independently selected from 0 to 2;
Z₁ and Z₂ are independently selected from a hydrophilic substituent group;
C and D are independently selected from aryl, heteroaryl, heterocyclyl, cycloalkyl, cycloalkenyl and a group of the formula: 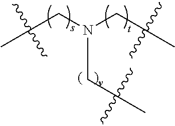
wherein:
s, t and v are integers independently selected from 1 or 2; [symbol]
denotes the point of attachment;
R₃ and R₄ are independently selected from halo, (1-4C)alkyl, (1-4C)alkoxy, amino, nitro, (1-4C)alkylamino, (1-4C)dialkylamino, (1-4C)haloalkyl, (1-4C)haloalkoxy, cyano, (2-4C)alkenyl, (2-4C)alkynyl and a group of the formula:
-L¹-Y¹-Q¹
wherein:
L¹ is absent or a (1-5C)alkylene optionally substituted by one or more substituents selected from (1-2C)alkyl and oxo;
Y¹ is absent or selected from a one of the following groups; O, S, SO, SO₂, N(Rₐ), C(O), C(O)O, OC(O), C(O)N(Rₐ), N(Rₐ)C(O), N(R_{b})C(O)N(Rₐ), N(Rₐ)C(O)O, OC(O)N(Rₐ), S(O)₂N(Rₐ), and N(Rₐ)SO₂, wherein Rₐ and R_{b} are each independently selected from hydrogen and (1-4C)alkyl; and
Q₁ is hydrogen, (1-8C)alkyl, (2-6C)alkenyl, (2-6C)alkynyl, aryl, (3-10C)cycloalkyl, (3-10C)cycloalkenyl, heteroaryl and heterocyclyl; wherein Q₁ is optionally further substituted by one or more substituent groups independently selected from (1-4C)alkyl, halo, (1-4C)haloalkyl, (1-4C)haloalkoxy, amino, (1-4C)aminoalkyl, cyano, hydroxy, carboxy, carbamoyl, sulfamoyl, mercapto, ureido, oxy, NR_{c}R_{d}, OR_{c}, C(O)R_{d}, C(O)OR_{c}, OC(O)R_{c}, C(O)N(R_{d})R_{c}, N(R_{d})C(O)R_{c}, S(O)_{y}R_{c} (where y is 0, 1, or 2), SO₂N(R_{d})R_{c}, N(R_{d})SO₂R_{c}, Si(Rₑ)(R_{d})R_{c} and (CH₂)_{z}NR_{d}R_{c} (where z is 1, 2 or 3); wherein R_{c}, R_{d} and Rₑ are each independently selected from hydrogen, (1-6C)alkyl and (3-6C)cycloalkyl; and R_{c} and R_{d} can be linked such that, together with the nitrogen atom to which they are attached, they form a 4-7 membered heterocyclic ring which is optionally substituted by one or more substituents selected from (1-4C)alkyl, halo, (1-4C)haloalkyl, (1-4C)haloalkoxy, (1-4C)alkoxy, (1-4C)alkylamino, amino, cyano or hydroxyl; and wherein
two R₃ and/or two R₄ groups taken together may form a group of the formula: 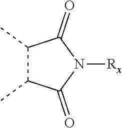
wherein:
Rₓ is selected from hydrogen and (1-6C)alkyl optionally substituted by one or more substituent groups selected from halo, (1-4C)haloalkyl, (1-4C)haloalkoxy, cyano, hydroxy, sulfamoyl, mercapto, ureido, NR_{f}R_{g}, OR_{f}, C(O)R_{f}, C(O)OR_{f}, OC(O)R_{f}, C(O)N(R_{g})R_{f} and N(R_{g})C(O)R_{f}, wherein R_{f} and R_{g} are selected from hydrogen and (1-4C)alkyl; and
the dashed lines represent the points of attachment to C and/or D;
W₁, W₂, W₃ and W₄ are independently selected from CRₕRᵢ, wherein Rₕ and Rᵢ, are selected from hydrogen and (1-2C)alkyl;
X₁, X₂, X₃ and X₄ are independently selected from a group of the formula: 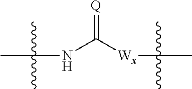
wherein: [symbol]
denotes the point of attachment;
Wₓ is selected from O or NH; and
Q is selected from O, S and NRⱼ, wherein Rⱼ is selected from hydrogen, (1-4C)alkyl, aryl, heteroaryl and sulfonyl;
Z₃ and Z₄ are independently selected from a hydrophilic substituent group;
L is absent or a linker, which optionally bears a hydrophilic substituent group Z₅;
c and d are integers independently selected from 0 to 4; and
o and p are integers independently selected from 0 to 2;
and wherein:
A. The sensing system includes an aqueous solution or dispersion including the saccharide-binding compound and the detectable reporter, the detectable reporter is a displaceable cavity-binding ligand including a fluorescent reporter moiety and a cavity-binding moiety for binding the cavity of the saccharide-binding compound, and wherein the compound of Formula I optionally includes one or more macrocycle tether groups at a position associated with one or more of the substituent groups R₁ₐ, R_{1b}, R₂ₐ, R_{2b}, R₁, R₂, R₃, R₄, R₅, Z₁, Z₂, Z₃, Z₄ and/or Z₅, wherein the or each macrocycle tether group is selected from (i) a macrocycle membrane blocking group MMBG tether group including a macrocycle membrane blocking group and one or more macrocycle linker units (L_{Munit}) linking the macrocycle of the saccharide-binding compound and the macrocycle membrane blocking group MMBG; (ii) a terminal tether group including a terminal group T_{MT} and one or more macrocycle linker units (L_{Munit}) linking the macrocycle of the saccharide-binding compound and the terminal group T_{MT}; and (iii) a chromophore tether group including a chromophore and one or more macrocycle linker units (L_{Munit}) linking the macrocycle of the saccharide-binding compound and the chromophore;
OR
B. The sensing system includes an electrode and an aqueous solution containing the saccharide-binding compound and the detectable reporter, the detectable reporter is a displaceable cavity-binding ligand including a redox moiety capable of reversible oxidation and a cavity-binding moiety for binding the cavity of the saccharide-binding compound; and wherein the compound of Formula I optionally includes one or more macrocycle tether groups at a position associated with one or more of the substituent groups R₁ₐ, R_{1b}, R₂ₐ, R_{2b}, R₁, R₂, R₃, R₄, R₅, Z₁, Z₂, Z₃, Z₄ and/or Z₅, wherein the or each macrocycle tether group is selected from (i) a macrocycle membrane blocking group MMBG tether group including a macrocycle membrane blocking group and one or more macrocycle linker units (L_{Munit}) linking the macrocycle of the saccharide-binding compound and the macrocycle membrane blocking group MMBG; and (ii) a terminal tether group including a terminal group T_{MT} and one or more macrocycle linker units (L_{Munit}) linking the macrocycle of the saccharide-binding compound and the terminal group T_{MT};
OR
C. The sensing system includes a semi-solid or solid support, wherein the saccharide-binding compound is immobilized on the semi-solid or solid support, the detectable reporter is a displaceable cavity-binding ligand independently immobilized on the semi-solid or solid support, where the a displaceable cavity-binding ligand includes a fluorescent reporter moiety and a cavity-binding moiety for binding the cavity of the saccharide-binding compound, and wherein the compound of Formula I includes a support tether group at a position associated with one or more of the substituent groups R₁ₐ, R_{1b}, R₂ₐ, R_{2b}, R₁, R₂, R₃, R₄, R₅, Z₁, Z₂, Z₃, Z₄ and/or Z₅, the support tether group including one or more macrocycle linker units (L_{Munit}) linking the macrocycle of the saccharide-binding compound and a semi-solid or solid support; and the compound of Formula (I) optionally further includes one or more macrocycle tether groups at a position associated with one or more of the substituent groups R₁ₐ, R_{1b}, R₂ₐ, R_{2b}, R₁, R₂, R₃, R₄, R₅, Z₁, Z₂, Z₃, Z₄ and/or Z₅, wherein the or each macrocycle tether group is selected from (i) a terminal tether group including a terminal group T_{MT} and one or more macrocycle linker units (L_{Munit}) linking the macrocycle of the saccharide-binding compound and the terminal group T_{MT}; and (ii) a chromophore tether group including a chromophore and one or more macrocycle linker units (L_{Munit}) linking the macrocycle of the saccharide-binding compound and the chromophore;
OR
D. the saccharide-binding compound includes the detectable reporter, the detectable reporter being a displaceable cavity-binding group including a fluorescent reporter moiety and a cavity-binding moiety for binding the cavity of the saccharide-binding compound, wherein the compound includes a cavity-binding tether group at a position associated with one or more of the substituent groups R₁ₐ, R_{1b}, R₂ₐ, R_{2b}, R₁, R₂, R₃, R₄, R₅, Z₁, Z₂, Z₃, Z₄ and/or Z₅, wherein the cavity-binding substituent group includes a fluorescent reporter moiety and a displaceable cavity-binding moiety for binding the cavity of a saccharide-binding compound; and wherein the compound of Formula I optionally includes one or more macrocycle tether groups at a position associated with one or more of the substituent groups R₁ₐ, R_{1b}, R₂ₐ, R_{2b}, R₁, R₂, R₃, R₄, R₅, Z₁, Z₂, Z₃, Z₄ and/or Z₅, wherein the or each macrocycle tether group is selected from (i) a macrocycle membrane blocking group MMBG tether group including a macrocycle membrane blocking group and one or more macrocycle linker units (L_{Munit}) linking the macrocycle of the saccharide-binding compound and the macrocycle membrane blocking group MMBG; (ii) a terminal tether group including a terminal group T_{MT} and one or more macrocycle linker units (L_{Munit}) linking the macrocycle of the saccharide-binding compound and the terminal group T_{MT}; and (iii) a chromophore tether group including a chromophore and one or more macrocycle linker units (L_{Munit}) linking the macrocycle of the saccharide-binding compound and the chromophore.

2. (canceled)

3. The saccharide sensing system according to claim 1C wherein the support tether group includes macrocycle linker units (L_{Munit}) linking the macrocyle to the semi-solid or solid support and the macrocycle linker units include one or more linear linkers independently selected from the group consisting of O, S, SO, SO₂, N(R^{x4}), C(O), C(O)O, OC(O), C(O)N(R^{x4}), N(R^{x4})C(O), N(R^{x4})C(O)N(R^{x5}), N(R^{x4})C(O)O, OC(O)N(R^{x4}), S(O)₂N(R^{x4}), N(R^{x4})SO₂, amino acids, (1-20C)alkylene, (1-20C)alkylene oxide, (1-20C)alkenyl, (1-20C)alkynyl, aryl, (3-1° C.)cycloalkyl, (3-10C)cycloalkenyl, heteroaryl and heterocyclyl, wherein R^{x4} and R^{x5} are each independently selected from hydrogen and (1-4C)alkyl.

4. The saccharide sensing system according to claim 3 wherein the support tether group further includes one or more macrocycle functional group selected from a terminal group T_{MT} and a chromophore, and one or more of the linear linkers is either substituted with one of the macrocycle tether functional groups or one or more of the linear linkers is replaced with a branched linker, wherein each branched linker is a linear linker substituted with one or more linear linkers and linking the branched linker to one of the functional groups.

5. The saccharide sensing system according to claim 1C, wherein the semi-solid or solid support is a polymeric matrix and/or a gel, such as a hydrogel.

6. The saccharide sensing system according to claim 1D, wherein the cavity-binding tether group is represented by:
L_{CB1}-fluorescent reporter moiety-L_{CB2}-displaceable cavity binding moiety;
where L_{CB1} is a first set of linker units, L_{CB2} is a second set of linker units; or wherein L_{CB1} and L_{CB2} include one or more linear linkers independently selected from the group consisting of O, S, SO, SO₂, N(R^{x4}), C(O), C(O)O, OC(O), C(O)N(R^{x4}), N(R^{x4})C(O), N(R^{x4})C(O)N(R^{x5}), N(R^{x4})C(O)O, OC(O)N(R^{x4}), S(O)₂N(R^{x4}), N(R^{x4})SO₂, amino acids, (1-20C)alkylene, (1-20C)alkylene oxide, (1-20C)alkenyl, (1-20C)alkynyl, aryl, (3-10C)cycloalkyl, (3-10C)cycloalkenyl, heteroaryl and heterocyclyl, wherein R^{x4} and R^{x5}are each independently selected from hydrogen and (1-4C)alkyl; or wherein the cavity-binding tether group further includes one or more macrocycle functional group selected from macrocycle membrane blocking group MMBG, a terminal group T_{MT} and a chromophore, and one or more of the linear linkers is either substituted with one of the macrocycle tether functional groups or one or more of the linear linkers is replaced with a branched linker, wherein each branched linker is a linear linker substituted with one or more linear linkers and linking the branched linker to one of the functional groups.

7. (canceled)

8. (canceled)

9. The saccharide sensing system according to claim 1D, wherein the fluorescent reporter moiety of the cavity-binding tether group is selected from xanthene dyes or derivatives; cyanine dyes or derivatives; squaraine dyes or derivatives and ring-substituted squaraines; squaraine rotaxane dyes or derivatives; naphthalene dyes or derivatives; coumarin dyes or derivatives; oxadiazole dyes or derivatives; anthracene dyes or derivatives; pyrene dyes or derivatives; oxazine dyes or derivatives; acridine dyes or derivatives; arylmethine dyes or derivatives; tetrapyrrole dyes or derivatives; dipyrromethene dyes or derivatives, metal coordination complexes or organometallic fluorophores, fluorescent dyes in the Alexa Fluor™ series and quinoline dyes or derivatives; or wherein the fluorescent reporter moiety is selected from selected from xanthene dyes or derivatives; cyanine dyes or derivatives; squaraine dyes or derivatives and ring-substituted squaraines; squaraine rotaxane dyes or derivatives; naphthalene dyes or derivatives; coumarin dyes or derivatives; oxadiazole dyes or derivatives; anthracene dyes or derivatives; pyrene dyes or derivatives; oxazine dyes or derivatives; acridine dyes or derivatives; arylmethine dyes or derivatives; tetrapyrrole dyes or derivatives; dipyrromethene dyes or derivatives, metal coordination complexes or organometallic fluorophores, fluorescent dyes in the Alexa Fluor™ series, and quinoline dyes or derivatives.

10. (canceled)

11. The saccharide sensing system according to claim 1A, 1B, or 1D, wherein the saccharide-binding compound includes the macrocycle membrane blocking group MMBG tether group at a position associated with one or more of the substituent groups R₁ₐ, R_{1b}, R₂ₐ, R_{2b}, R₁, R₂, R₃, R₄, R₅, Z₁, Z₂, Z₃, Z₄ and/or Z₅.

12. The saccharide sensing system according to claim 11 wherein the macrocycle membrane blocking group MMBG has a molecular weight of at least about 1,000 daltons (1 kDa), at least about 2,000 daltons (2 kDa), at least about 5,000 daltons (5 kDa), at least about 10,000 daltons (10 kDa) or at least about 30,000 daltons (kDa).

13. The saccharide sensing system according to claim 11 the macrocycle membrane blocking group MMBG is a water-soluble polymer, such as water-soluble polysaccharides, polyacrylamides, polyethylene glycol, peptides and nucleic acids.

14. The saccharide sensing system according to claim 11, wherein the macrocycle linker units (L_{Munit}) of the macrocycle membrane blocking group MMBG tether group link the macrocycle to the MMBG and the macrocycle linker units (L_{Munit}) include of one or more linear linkers independently selected from the group consisting of O, S, SO, SO₂, N(R^{x4}), C(O), C(O)O, OC(O), C(O)N(R^{x4}), N(R^{x4})C(O), N(R^{x4})C(O)N(R^{x5}), N(R^{x4})C(O)O, OC(O)N(R^{x4}), S(O)₂N(R^{x4}), N(R^{x4})SO₂, amino acids, (1-20C)alkylene, (1-20C)alkylene oxide, (1-20C)alkenyl, (1-20C)alkynyl, aryl, (3-10C)cycloalkyl, (3-10C)cycloalkenyl, heteroaryl and heterocyclyl, wherein R^{x4} and R^{x5} are each independently selected from hydrogen and (1-4C)alkyl.

15. The saccharide sensing system according to claim 14 wherein the macrocycle membrane blocking group MMBG tether group further includes one or more of a further macrocycle membrane blocking groups MMBG, a terminal group T_{MT}, and/or a chromophore; or wherein the macrocycle membrane blocking group MMBG tether group includes one or more of the linear linkers and one or more of the linear linkers is either substituted with one of the macrocycle tether functional groups or one or more of the linear linkers is replaced with a branched linker, wherein each branched linker is a linear linker substituted with one or more linear linkers and linking the branched linker to the further macrocycle membrane blocking groups MMBG, a terminal group TMT, and/or a chromophore.

16. (canceled)

17. The saccharide sensing system according to claim 1A, 1C, or 1D, wherein the saccharide-binding compound includes the chromophore tether group; or wherein the macrocycle linker units (L_{Munit}) of the chromophore tether group link the macrocycle to the chromophore and the macrocycle linker units (L_{Munit}) include of one or more linear linkers independently selected from the group consisting of O, S, SO, SO₂, N(R^{x4}), C(O), C(O)O, OC(O), C(O)N(R^{x4}), N(R^{x4})C(O), N(R^{x4})C(O)N(R^{x5}), N(R^{x4})C(O)O, OC(O)N(R^{x4}), S(O)₂N(R^{x4}), N(R^{x4})SO₂, amino acids, (1-20C)alkylene, (1-20C)alkylene oxide, (1-20C)alkenyl, (1-20C)alkynyl, aryl, (3-10C)cycloalkyl, (3-10C)cycloalkenyl, heteroaryl and heterocyclyl, wherein R^{x4} and R^{x5}are each independently selected from hydrogen and (1-4C)alkyl; or wherein the chromophore is an energy transfer donor or acceptor, such as a Forster resonance energy transfer (FRET) or photoinduced energy transfer (PET) donor or acceptor.

18. (canceled)

19. (canceled)

20. The saccharide sensing system according to claim 1, wherein the saccharide-binding compound includes the terminal tether group; or wherein the terminal group TMT is either (i) capable of reacting with a semi-solid or solid support or precursors of a semi-solid or solid support or (ii) a solubilizing group for increasing the solubility of the saccharide-binding compound in a solvent.

21. The saccharide sensing system according to claim 20, wherein the macrocycle linker units (L_{Munit}) of the terminal tether group link the macrocycle to the terminal group T_{MT} and the macrocycle linker units (L_{Munit}) include of one or more linear linkers independently selected from the group consisting of O, S, SO, SO₂, N(R^{x4}), C(O), C(O)O, OC(O), C(O)N(R^{x4}), N(R^{x4})C(O), N(R^{x4})C(O)N(R^{x5}), N(R^{x4})C(O)O, OC(O)N(R^{x4}), S(O)₂N(R^{x4}), N(R^{x4})SO₂, amino acids, (1-20C)alkylene, (1-20C)alkylene oxide, (1-20C)alkenyl, (1-20C)alkynyl, aryl, (3-10C)cycloalkyl, (3-10C)cycloalkenyl, heteroaryl, heterocyclyl, peptides (such as oligopeptides or polypeptides) and nucleic acids (such as DNA or RNA), wherein R^{x4} and R^{x5} are each independently selected from hydrogen and (1-4C)alkyl.

22. (canceled)

23. The saccharide sensing system according to claim 1A or 1B, wherein the cavity-binding ligand includes a terminal group T_{L} and/or ligand membrane blocking group LMBG, and either the terminal group T_{L} and/or ligand membrane blocking group LMBG being substituted onto one of the linker units linking the cavity-binding moiety to the fluorescent reporter moiety or the redox moiety, or one or more of the linker units linking the cavity-binding moiety to the fluorescent reporter moiety or the redox moiety is replaced with a branched linker, wherein each branched linker is a linear linker as described herein substituted with one or more linear linkers as described herein and linking the branched linker to terminal group T_{L} and/or ligand membrane blocking group.

24. The saccharide sensing system according to claim 23 wherein the cavity-binding ligand includes a ligand membrane blocking group LMBG having a molecular weight of at least about 1,000 daltons (1 kDa), at least about 2,000 daltons (2 kDa), at least about 5,000 daltons (5 kDa), at least about 10,000 daltons (10 kDa) or at least about 30,000 daltons (30 kDa).

25. The saccharide sensing system according to claim 23 wherein the cavity-binding ligand includes a water soluble polymer, such as water soluble polysaccharides, polyacrylamides, polyethylene glycol, peptides and nucleic acids as the ligand membrane blocking group LMBG.

26. (canceled)

27. The saccharide sensing system according to claim 1B or 1C, wherein the displaceable cavity-binding ligand includes one or more ligand linker units (L_{Lunit}) linking the cavity-binding moiety and fluorescent reporter moiety or the redox moiety.

28. The saccharide sensing system according to claim 1, wherein the system is housed in a detection chamber

29. The saccharide sensing system according to claim 29, wherein the detection chamber the detection chamber includes at least one interrogation window for transmitting excitation radiation into the detection chamber and/or receiving a detectable signal from the detection chamber; or wherein the detection chamber has an inlet for the ingress of the target saccharide into the aqueous environment of the system and the inlet is a selectively permeable membrane; or wherein the selectively permeable membrane is a molecular weight cut off (MWCO) membrane, the molecular weight cut off being lower than the molecular weight of the saccharide-binding compound and/or the displaceable cavity-binding ligand when present.

30. (canceled)

31. (canceled)

32. The saccharide sensing system according to claim 1, wherein the cavity-binding moiety is selected so that cavity-binding moiety is displaced from the cavity of the saccharide-binding compound when the glucose sensing system is contacted with the target saccharide-containing aqueous environment; or wherein the cavity-binding moiety includes a saccharide, such as a glucoside or a glycosid.

33. (canceled)

34. (canceled)

35. The saccharide sensing system according to claim 1, wherein the cavity-binding moiety includes salidroside or a glucoronide.

36. The saccharide sensing system according to claim 1, wherein the target saccharide is glucose.

37. The saccharide sensing system according to claim 1, wherein the saccharide-binding compound includes at least one hydrophilic substituent group.

38. The saccharide sensing system according to claim 1, wherein the saccharide-binding compound is a compound of formula II shown below: 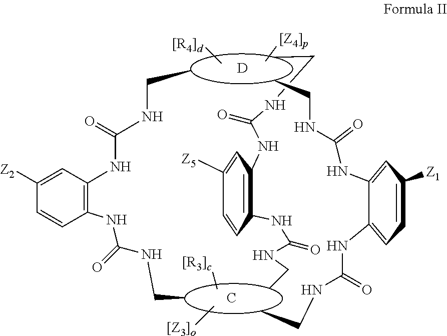
Where each of C, D, R₃, R₄, Z₁, Z₂, Z₃, Z₄, Z₅, c, d, o and p are as defined in claim 1.

39. The saccharide sensing system according to claim 1, wherein one or more of Z₁, Z₂, Z₃, Z₄, Z₅ are independently selected from a hydrophilic substituent group comprising one or more hydrophilic functional groups selected from carboxylic acids, carboxylate ions, carboxylate esters, hydroxyl, amines, amides, ethers, ketone and aldehyde groups, nitro groups, sulphates, sulphonates, phosphates, phosphonates, and combinations thereof.

40. An implantable device including a saccharide sensing system according to claim 1.

41. A method of detecting a target saccharide with a saccharide sensing system according to claim 1, wherein the method includes:
(i) contacting the saccharide sensing system with a target aqueous environment; and
(ii) measuring a detectable signal from the detectable reporter of the system.

42. A method of detecting of abnormal target saccharide in a subject's biological fluid, the method of detection includes:
(i) detecting a target saccharide in a subject's biological fluid with a saccharide sensing system according to claim 1;
(ii) Comparing one or more detectable signal measured from the detectable reporter of the system with a standard or calibration value; and
(iii) Detecting an abnormal level of target saccharide in the subject's biological fluid based on the comparison in step (ii).
